(19) United States
(12) Patent Application Publication
(10) Pub. No.: US 2024/0381763 A1
(43) Pub. Date: Nov. 14, 2024

(54) NOVEL COMPOUND AND ORGANIC LIGHT EMITTING DEVICE COMPRISING THE SAME

(71) Applicant: LG CHEM, LTD., Seoul (KR)

(72) Inventors: Minjun KIM, Daejeon (KR); Dong Hoon LEE, Daejeon (KR); Sang Duk SUH, Daejeon (KR); Min Woo JUNG, Daejeon (KR); Jungha LEE, Daejeon (KR); Su Jin HAN, Daejeon (KR)

(21) Appl. No.: 18/034,474

(22) PCT Filed: Feb. 16, 2022
(86) PCT No.: PCT/KR2022/002284

§ 371 Date: Apr. 28, 2023

(30) Foreign Application Priority Data
Feb. 16, 2021 (KR) 10-2021-0020507
Feb. 15, 2022 (KR) 10-2022-0019692

(51) Int. Cl. H10K 85/60; C07C 211/54; C07C 211/58; C07C 211/61; C07D 209/88; C07D 307/77; C07D 307/91; C07D 333/50; C07D 409/12; C09K 11/06; H10K 50/18

(52) U.S. Cl. CPC H10K 85/633; C07C 211/54; C07C 211/58; C07C 211/61; C07D 209/88; C07D 307/77; C07D 307/91; C07D 333/50; C07D 409/12; C09K 11/06; H10K 85/636; C07C 2603/18; C07C 2603/26; C07C 2603/42; C09K 2211/1003; C09K 2211/1018; H10K 50/181; H10K 85/615; H10K 85/626; H10K 85/657; H10K 85/6572; H10K 85/6574; H10K 85/6576

(57) ABSTRACT

Provided is a novel compound and an organic light emitting device comprising the same. A compound of Chemical Formula 1:

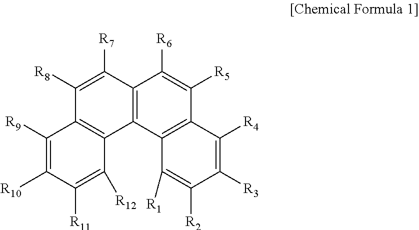

where any one of R₁ to R₁₂ is a substituent of Chemical Formula 2, and the rest are hydrogen or deuterium:

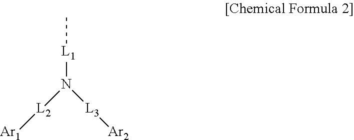

where L₁ is a substituted or unsubstituted phenylene, biphenyldiyl, or naphthalenediyl; L₂ and L₃ are each independently a single bond, or a substituted or unsubstituted C₆₋₆₀ arylene or C₂₋₆₀ heteroarylene containing at least one of N, O and S, and Ar₁ and Ar₂ are each independently a substituted or unsubstituted C₆₋₆₀ aryl or C₂₋₆₀ heteroaryl containing at least one of N, O and S may be used as a material for an organic material layer in the organic light emitting device, and may improve efficiency and properties in terms of a low driving voltage and a lifespan in the organic light emitting device.

### CROSS-REFERENCE TO RELATED APPLICATION(S)

This application is a National Stage Application of International Application No. PCT/KR2022/002284 filed on Feb. 16, 2022, which claims priority to and the benefit of Korean Patent Application No. 10-2021-0020507 filed on Feb. 16, 2021 and Korean Patent Application No. 10-2022-0019692 filed on Feb. 15, 2022 in the Korean Intellectual Property Office, the disclosures of which are incorporated herein by reference in their entirety.

### TECHNICAL FIELD

The present disclosure relates to a novel compound and an organic light emitting device comprising the same.

### BACKGROUND

In general, an organic light-emitting phenomenon refers to a phenomenon where electric energy is converted into light energy by using an organic material. The organic light emitting device using the organic light-emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer interposed between the anode and the cathode. The organic material layer frequently has a multilayered structure that comprises different materials in order to enhance efficiency and stability of the organic light emitting device, and for example, the organic material layer can be formed of a hole injection layer, a hole transport layer, a light emitting layer, an electron transport layer, an electron injection layer, and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

There is a need for continuous development of new materials for the organic materials used in the organic light emitting devices as described above.

### PRIOR ART LITERATURE

### Patent Literature

(Patent Literature 1) Korean Unexamined Patent Publication No. 10-2000-0051826

### BRIEF DESCRIPTION

### Technical Problem

It is an object of the present disclosure to provide a novel organic light-emitting material and an organic light emitting device comprising the same.

### Technical Solution

According to an aspect of the present disclosure, provided is a compound of Chemical Formula 1:

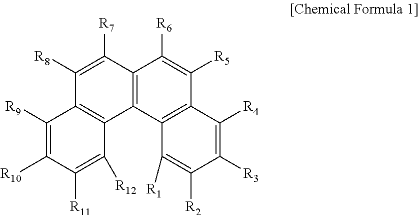

wherein in Chemical Formula 1:
any one of R₁ to R₁₂ is a substituent of Chemical Formula 2, and the rest are hydrogen or deuterium:

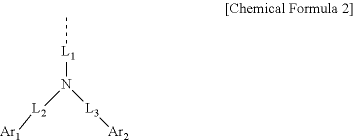

wherein in Chemical Formula 2:
L₁ is a substituted or unsubstituted phenylene, a substituted or unsubstituted biphenyldiyl, or a substituted or unsubstituted naphthalenediyl;
L₂ and L₃ are each independently a single bond, a substituted or unsubstituted C₆₋₆₀ arylene, or a substituted or unsubstituted C₂₋₆₀ heteroarylene containing at least one heteroatom selected from the group consisting of N, O and S; and
Ar₁ and Ar₂ are each independently a substituted or unsubstituted C₆₋₆₀ aryl, or a substituted or unsubstituted C₂₋₆₀ heteroaryl containing at least one heteroatom selected from the group consisting of N, O and S.

According to another aspect of the present disclosure, provided is an organic light emitting device comprising: a first electrode; a second electrode that is provided opposite to the first electrode; and one or more organic material layers that are provided between the first electrode and the second electrode, wherein one or more layers of the organic material layers include the compound of Chemical Formula 1.

### Advantageous Effects

The above-mentioned compound of Chemical Formula 1 can be used as a material for an organic material layer of an organic light emitting device, and can improve the efficiency, achieve low driving voltage and/or improve lifetime characteristics in the organic light emitting device. Particularly, the compound of Chemical Formula 1 described above can be used as a light-emitting material.

### BRIEF DESCRIPTION OF THE DRAWINGS

FIG. 1 shows an example of an organic light emitting device comprising a substrate 1, an anode 2, an electron blocking layer 3, a light emitting layer 4 and a cathode 5.

FIG. 2 shows an example of an organic light emitting device comprising a substrate 1, an anode 2, a hole injection layer 6, a hole transport layer 7, an electron blocking layer 3, a light emitting layer 4, a hole blocking layer 8, an electron transport and injection layer 9 and a cathode 5.

### DETAILED DESCRIPTION

Hereinafter, embodiments of the present disclosure will be described in more detail to facilitate understanding of the invention.

Provided herein is the compound of Chemical Formula 1.

As used herein, the notation [symbol] or [symbol] means a bond linked to another substituent group.

As used herein, the term “substituted or unsubstituted” means being unsubstituted or substituted with one or more substituents selected from the group consisting of deuterium, a halogen group, a nitrile group, a nitro group, a hydroxy group, a carbonyl group, an ester group, an imide group, an amino group, a phosphine oxide group, an alkoxy group, an aryloxy group, an alkylthioxy group, an arylthioxy group, an alkylsulfoxy group, an arylsulfoxy group, a silyl group, a boron group, an alkyl group, a cycloalkyl group, an alkenyl group, an aryl group, an aralkyl group, an aralkenyl group, an alkylaryl group, an alkylamine group, an aralkylamine group, a heteroarylamine group, an arylamine group, an arylphosphine group, and a heteroaryl group containing at least one of N, O and S atoms, or being unsubstituted or substituted with a substituent to which two or more substituents of the above-exemplified substituents are linked. For example, “a substituent in which two or more substituents are linked” can be a biphenyl group. Namely, a biphenyl group can be an aryl group, or it can also be interpreted as a substituent in which two phenyl groups are linked.

In the present disclosure, the carbon number of a carbonyl group is not particularly limited, but is preferably 1 to 40. Specifically, the carbonyl group can be a substituent group having the following structural formulas, but is not limited thereto:

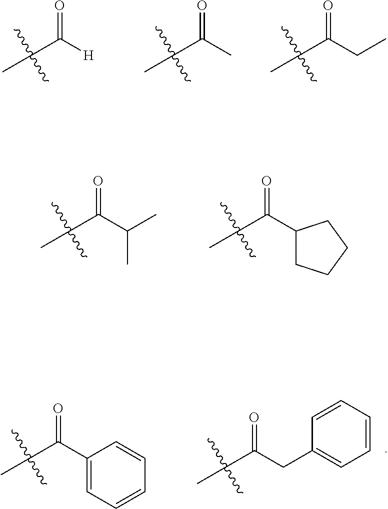

In the present disclosure, an ester group can have a structure in which oxygen of the ester group can be substituted by a straight-chain, branched-chain, or cyclic alkyl group having 1 to 25 carbon atoms, or an aryl group having 6 to 25 carbon atoms. Specifically, the ester group can be a substituent group having the following structural formulas, but is not limited thereto:

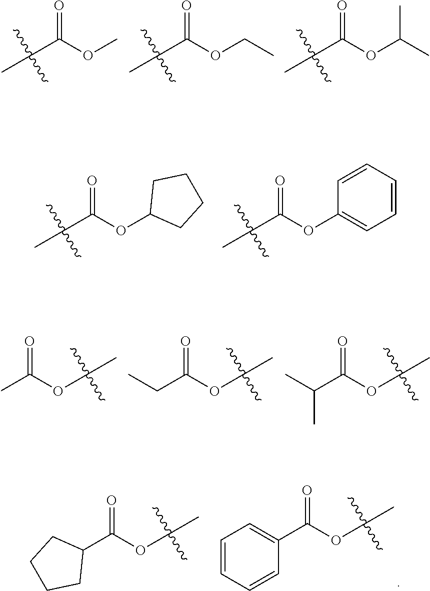

In the present disclosure, the carbon number of an imide group is not particularly limited, but is preferably 1 to 25. Specifically, the imide group can be a substituent group having the following structural formulas, but is not limited thereto:

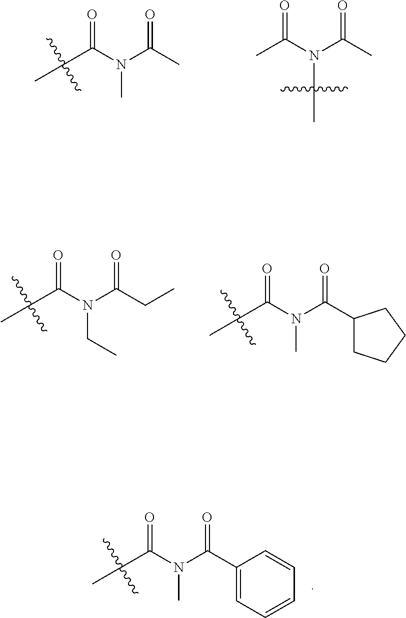

In the present disclosure, a silyl group specifically includes a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like, but is not limited thereto.

In the present disclosure, a boron group specifically includes a trimethylboron group, a triethylboron group, a t-butyldimethylboron group, a triphenylboron group, and a phenylboron group, but is not limited thereto.

In the present disclosure, examples of a halogen group include fluorine, chlorine, bromine, or iodine.

In the present disclosure, the alkyl group can be straight-chain or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. According to another embodiment, the carbon number of the alkyl group is 1 to 6. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 2-methylpentyl, 4-methylhexyl, 5-methylhexyl, and the like, but are not limited thereto.

In the present disclosure, the alkenyl group can be straight-chain or branched-chain, and the carbon number thereof is not particularly limited, but is preferably 2 to 40. According to one embodiment, the carbon number of the alkenyl group is 2 to 20. According to another embodiment, the carbon number of the alkenyl group is 2 to 10. According to still another embodiment, the carbon number of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present disclosure, a cycloalkyl group is not particularly limited, but the carbon number thereof is preferably 3 to 60. According to one embodiment, the carbon number of the cycloalkyl group is 3 to 30. According to another embodiment, the carbon number of the cycloalkyl group is 3 to 20. According to still another embodiment, the carbon number of the cycloalkyl group is 3 to 6. Specific examples thereof include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present disclosure, an aryl group is not particularly limited, but the carbon number thereof is preferably 6 to 60, and it can be a monocyclic aryl group or a polycyclic aryl group. According to one embodiment, the carbon number of the aryl group is 6 to 30. According to one embodiment, the carbon number of the aryl group is 6 to 20. The aryl group can be a phenyl group, a biphenyl group, a terphenyl group or the like as the monocyclic aryl group, but is not limited thereto. The polycyclic aryl group includes a naphthyl group, an anthracenyl group, a phenanthryl group, a pyrenyl group, a perylenyl group, a chrysenyl group, a fluorenyl group, or the like, but is not limited thereto.

In the present disclosure, the fluorenyl group can be substituted, and two substituents can be connected to each other to form a spiro structure. In the case where the fluorenyl group is substituted,

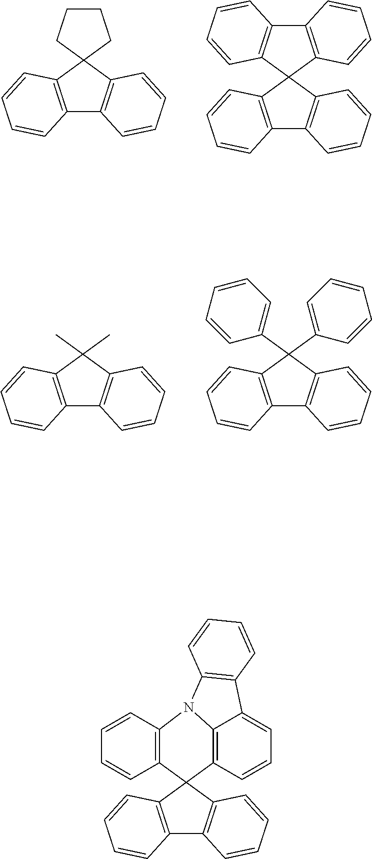

and the like can be formed. However, the structure is not limited thereto

In the present disclosure, a heteroaryl group is a heteroaryl group containing at least one of O, N, Si and S as a heteroatom, and the carbon number thereof is not particularly limited, but is preferably 2 to 60. According to an exemplary embodiment of heteroaryl, the heteroaryl group has 6 to 30 carbon atoms. According to an exemplary embodiment, the heteroaryl group has 6 to 20 carbon atoms. Examples of heteroaryl groups include a thiophene group, a furan group, a pyrrole group, an imidazole group, a thiazole group, an oxazol group, an oxadiazol group, a triazol group, a pyridyl group, a bipyridyl group, a pyrimidyl group, a triazine group, an acridyl group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, an indole group, a carbazole group, a benzoxazole group, a benzoimidazole group, a benzothiazol group, a benzocarbazole group, a benzothiophene group, a dibenzothiophene group, a benzofuranyl group, a phenanthroline group, an isoxazolyl group, a thiadiazolyl group, a phenothiazinyl group, a dibenzofuranyl group, and the like, but are not limited thereto.

In the present disclosure, the aryl group in the aralkyl group, the aralkenyl group, the alkylaryl group and the arylamine group is the same as the above-mentioned examples of the aryl group. In the present disclosure, the alkyl group in the aralkyl group, the alkylaryl group and the alkylamine group is the same as the above-mentioned examples of the alkyl group. In the present disclosure, the heteroaryl in the heteroarylamine can be applied to the above-mentioned description of the heteroaryl group. In the present disclosure, the alkenyl group in the aralkenyl group is the same as the above-mentioned examples of the alkenyl group. In the present disclosure, the above-mentioned description of the aryl group can be applied except that the arylene is a divalent group. In the present disclosure, the above-mentioned description of the heteroaryl group can be applied except that the heteroarylene is a divalent group. In the present disclosure, the above-mentioned description of the aryl group or cycloalkyl group can be applied except that the hydrocarbon ring is not a monovalent group but formed by combining two substituent groups. In the present disclosure, the above-mentioned description of the heteroaryl group can be applied, except that the heteroaryl is not a monovalent group but formed by combining two substituent groups.

Preferably, any one of R₁, R₃ to R₁₀ and R₁₂ is a substituent of Chemical Formula 2, the rest are each independently hydrogen or deuterium, and R₂ and R₁₁ can be each independently hydrogen or deuterium. Preferably, any one of R₁, R₃ to R₁₀ and R₁₂ is a substituent of Chemical Formula 2, the rest are each independently hydrogen, and R₂ and R₁₁ can be each independently hydrogen.

Preferably, L₁ can be phenylene that is unsubstituted or substituted with one phenyl; a biphenyldiyl that is unsubstituted or substituted with one phenyl; or a naphthalendiyl.

More preferably, L₁ can be any one selected from the group consisting of:

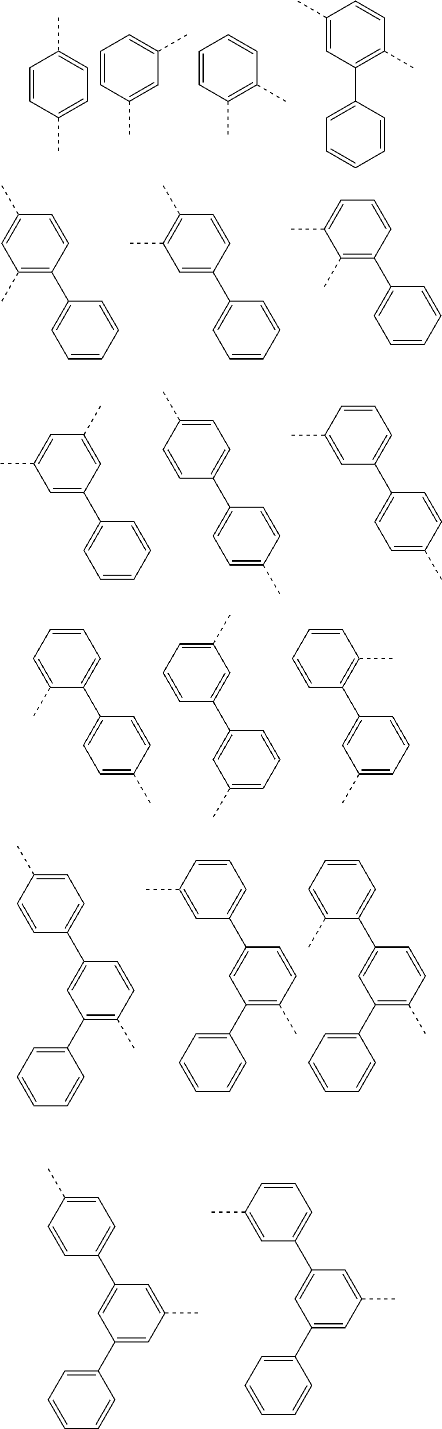

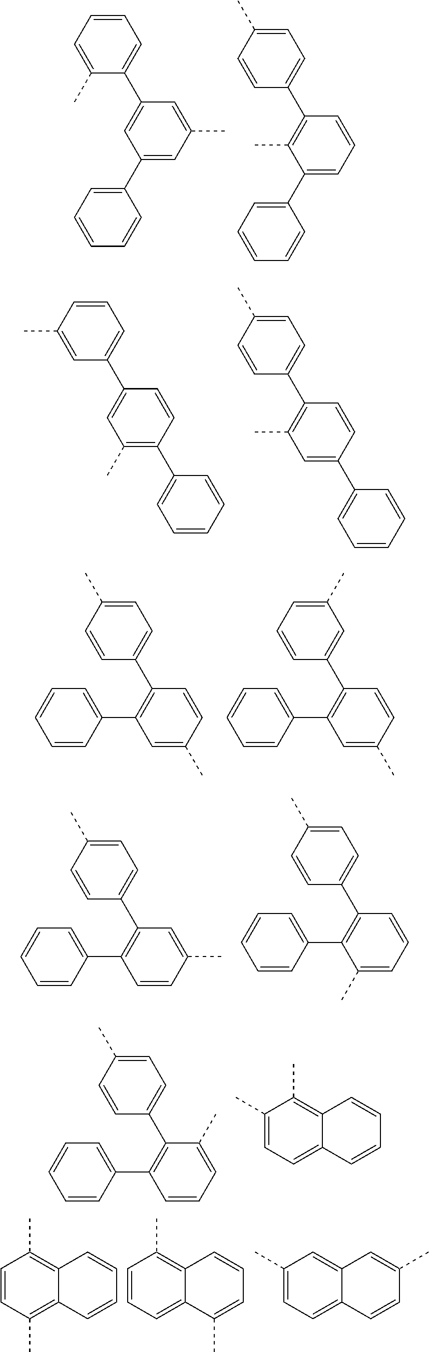

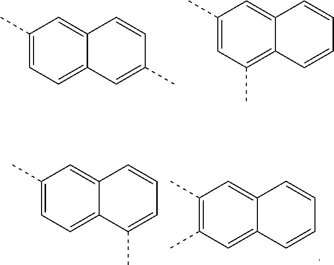

More preferably, L₁ can be any one selected from the group consisting of:

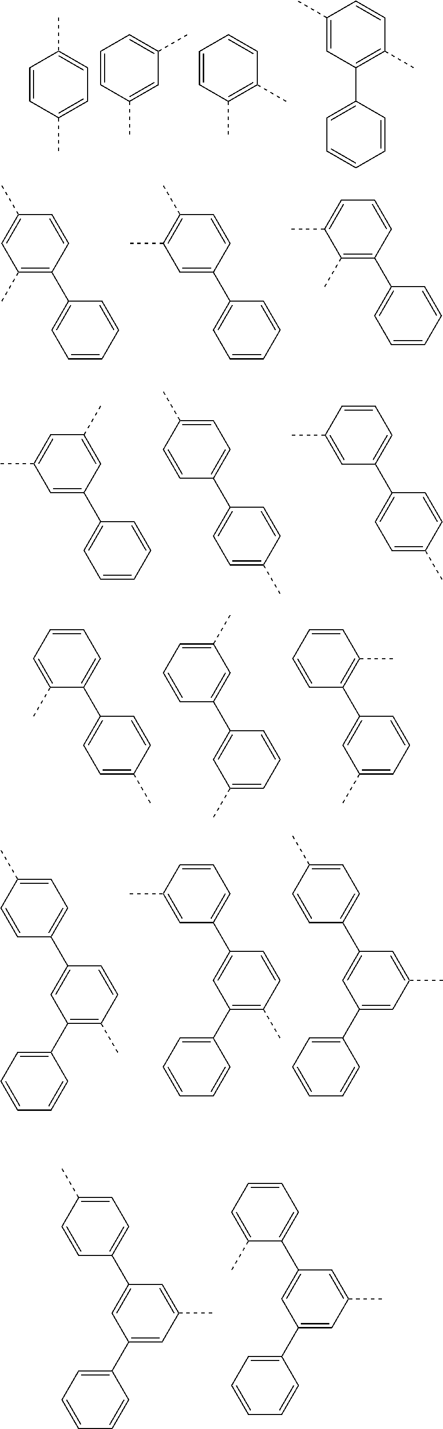

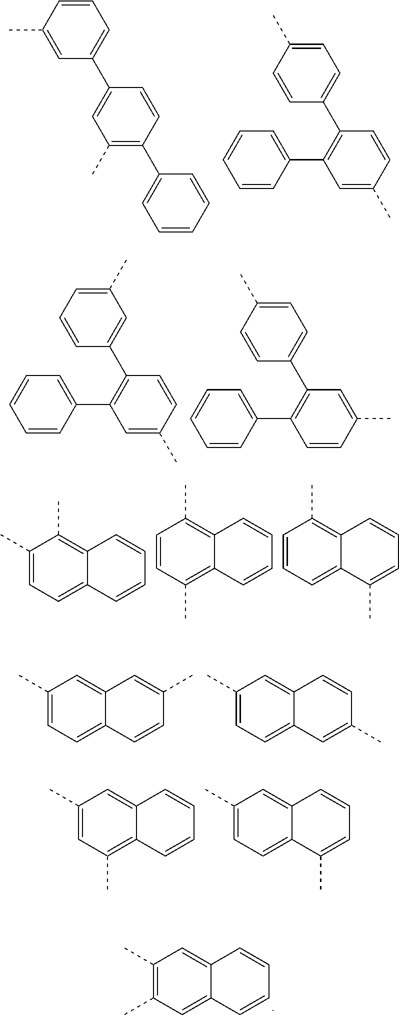

Preferably, L₂ and L₃ can be each independently a single bond, a substituted or unsubstituted C₆₋₂₀ arylene, or a substituted or unsubstituted C₂₋₂₀ heteroarylene containing at least one heteroatom selected from the group consisting of N, O and S.

More preferably, L₂ and L₃ can be each independently a single bond, phenylene, biphenyldiyl, or naphthalenediyl, and still more preferably, L₂ and L₃ are each independently a single bond, a phenylene, a phenylene substituted with one phenyl, a biphenyldiyl, or a naphthalenediyl.

Most preferably, L₂ and L₃ can be each independently a single bond or any one selected from the group consisting of:

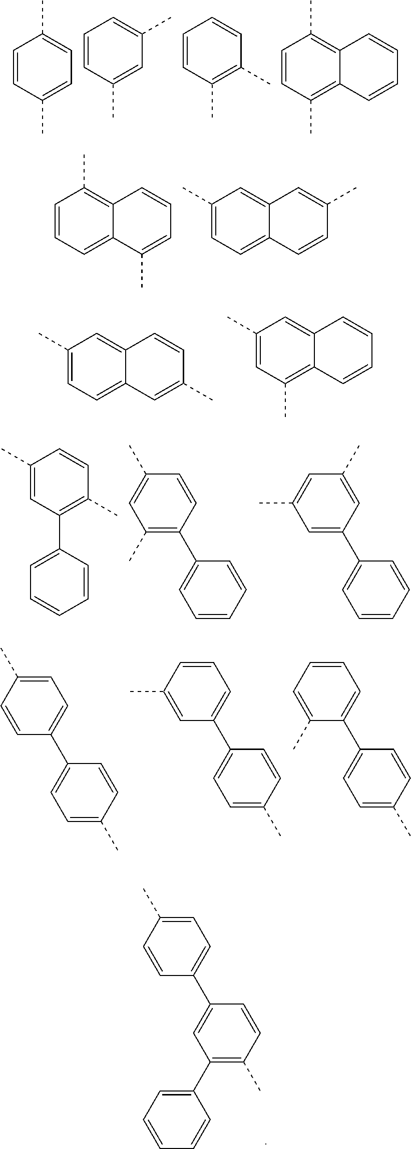

Preferably, Ar₁ and Ar₂ can be each independently a substituted or unsubstituted C₆₋₂₀ aryl, or a substituted or unsubstituted C₂₋₂₀ heteroaryl containing at least one heteroatom selected from the group consisting of N, O and S.

More preferably, Ar₁ and Ar₂ can be each independently phenyl, biphenylyl, terphenylyl, naphthyl, phenanthrenyl, phenyl naphthyl, dibenzofuranyl, dibenzothiophenyl, phenyl carbazolyl, dimethyl fluorenyl, benzonaphthofuranyl, or benzonaphthothiophenyl.

More preferably, Ar₁ and Ar₂ can be each independently any one selected from the group consisting of:

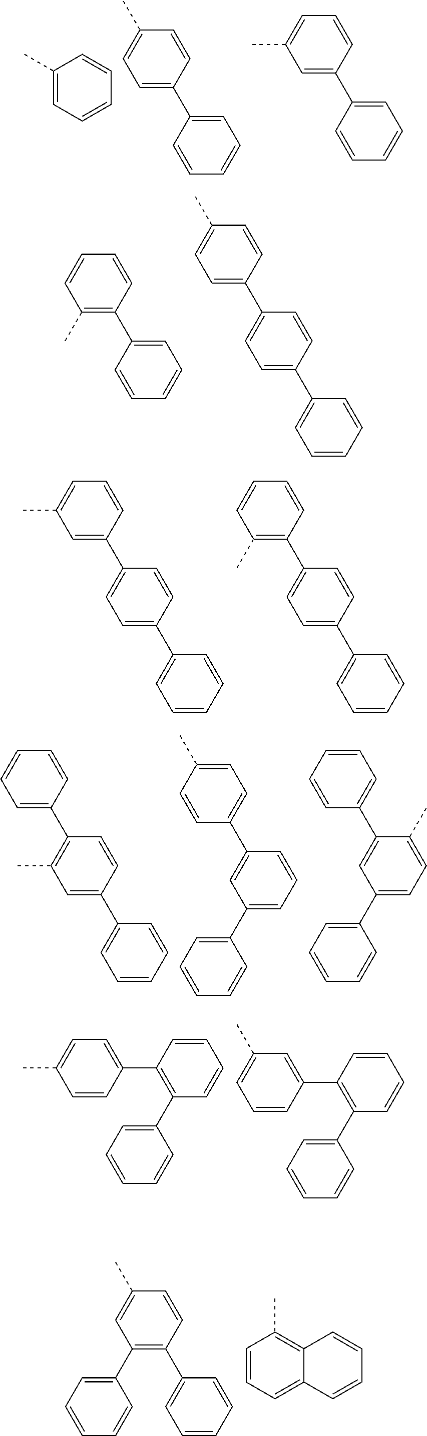

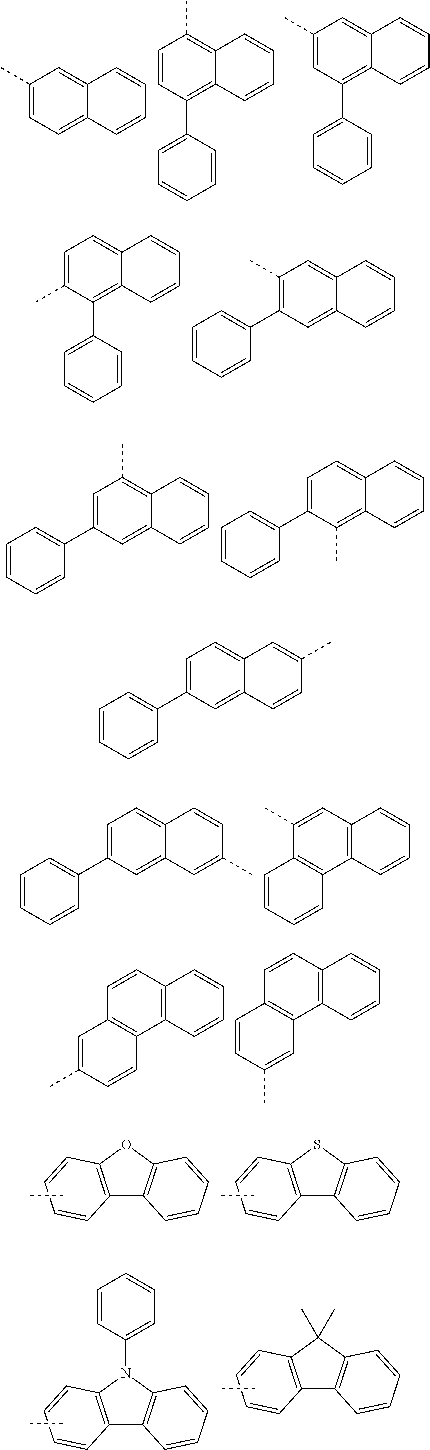

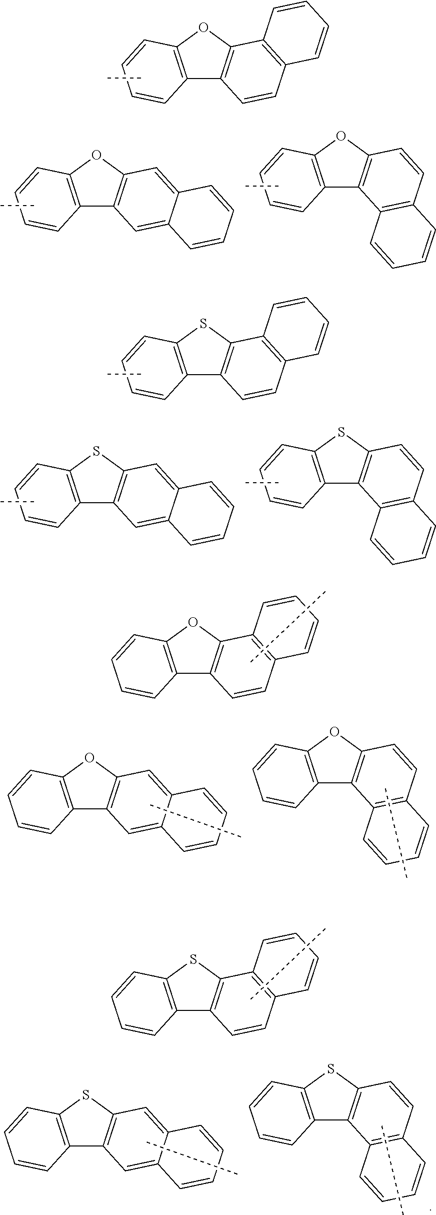

Most preferably, Ar₁ and Ar₂ can be each independently any one selected from the group consisting of:

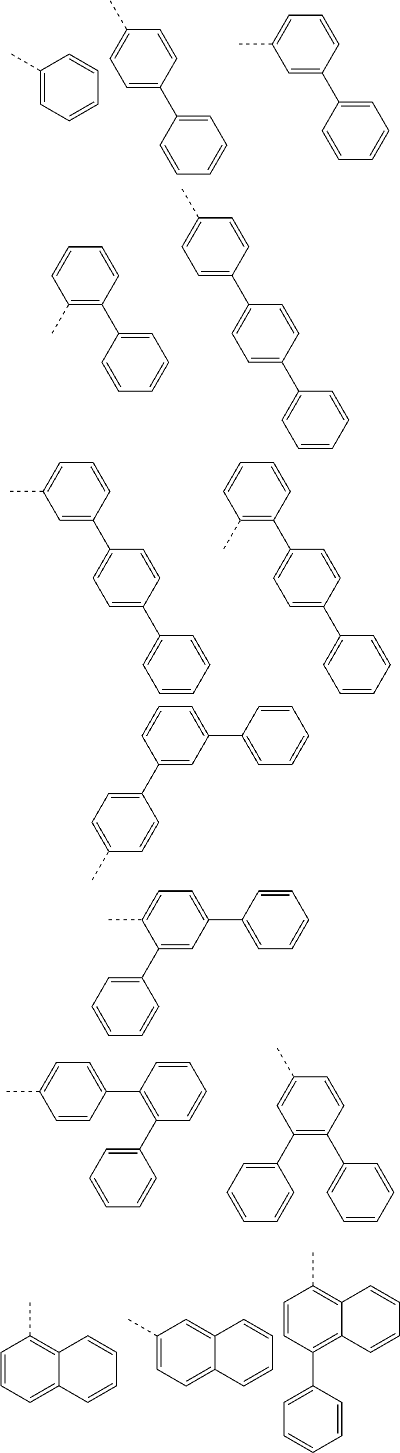

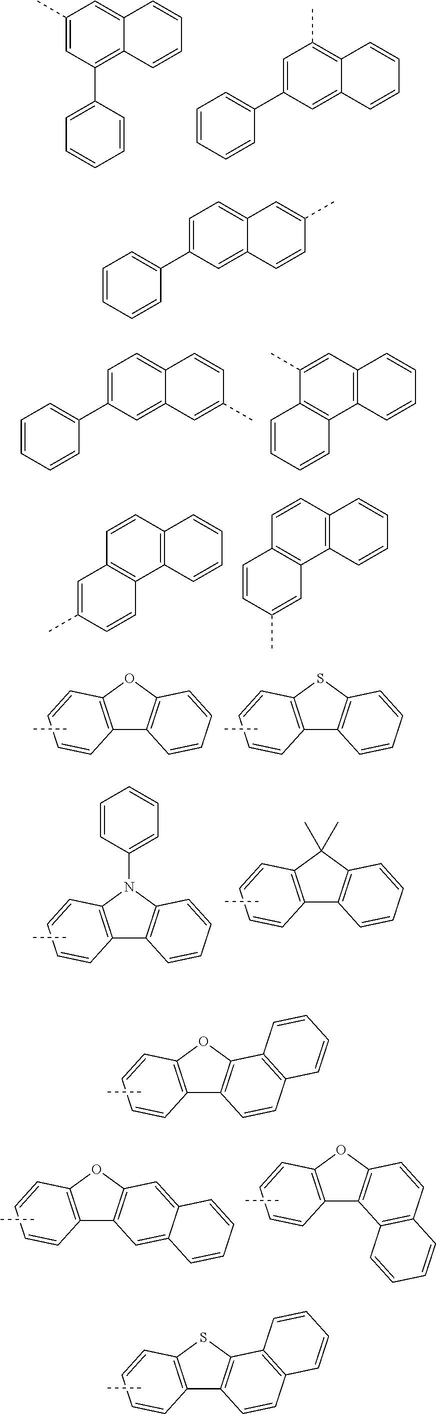

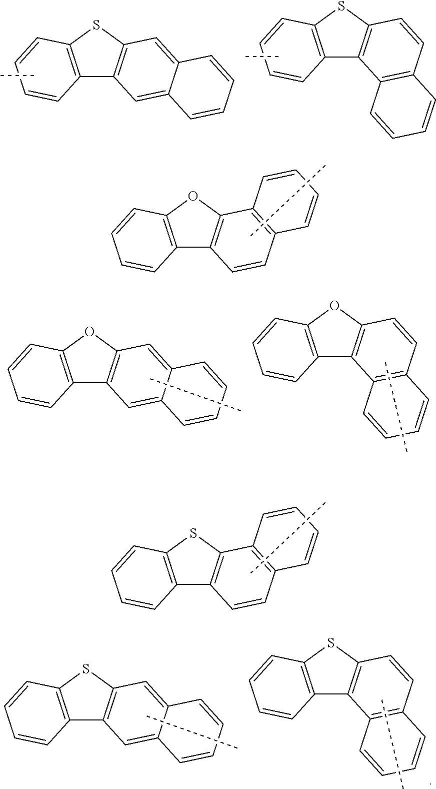

Preferably, any one of Ar₁ and Ar₂ can be a substituted or unsubstituted C₆₋₆₀ aryl. More preferably, any one of Ar₁ and Ar₂ can be a substituted or unsubstituted C₆₋₂₀ aryl. Still more preferably, any one of Ar₁ and Ar₂ can be phenyl, biphenylyl, terphenylyl, naphthyl, phenanthrenyl, or dimethyl fluorenyl.

Most preferably, any one of Ar₁ and Ar₂ can be any one selected from the group consisting of:

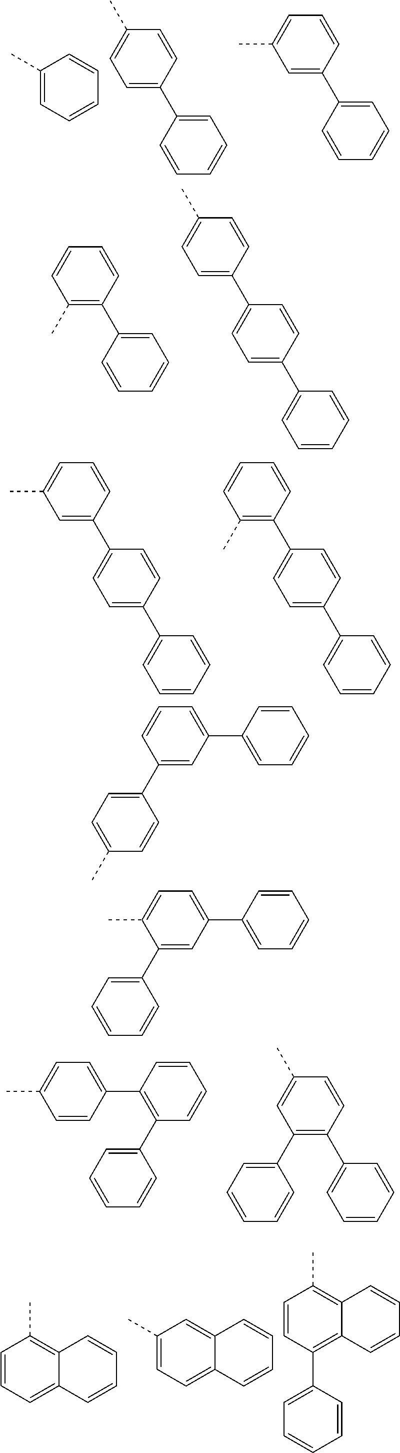

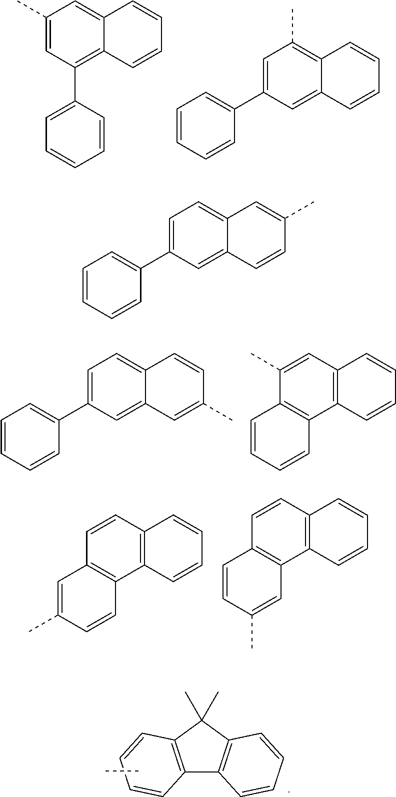

Representative examples of the compound of Chemical Formula 1 are as follows:

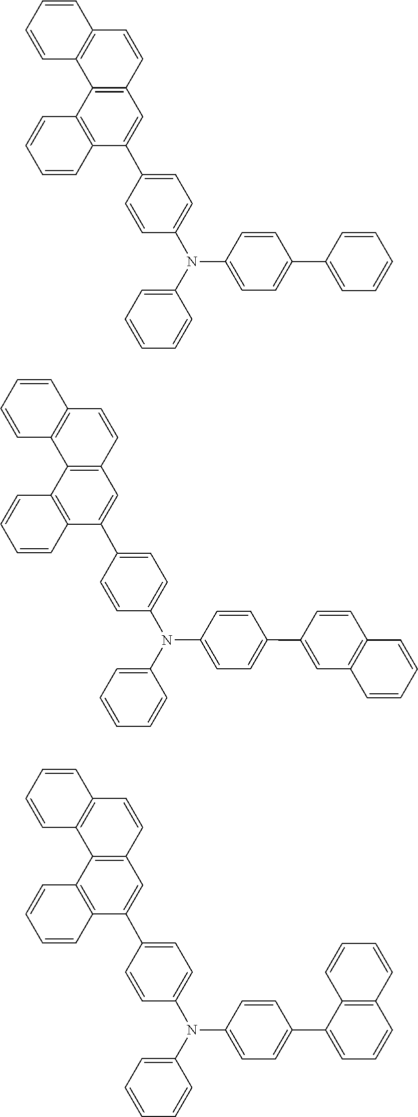 [SMILES: c1ccc(-c2ccc(N(c3ccccc3)c3ccc(-c4cc5ccc6ccccc6c5c5ccccc45)cc3)cc2)cc1 ; c1ccc(N(c2ccc(-c3ccc4ccccc4c3)cc2)c2ccc(-c3cc4ccc5ccccc5c4c4ccccc34)cc2)cc1 ; c1ccc(N(c2ccc(-c3cccc4ccccc34)cc2)c2ccc(-c3cc4ccc5ccccc5c4c4ccccc34)cc2)cc1]

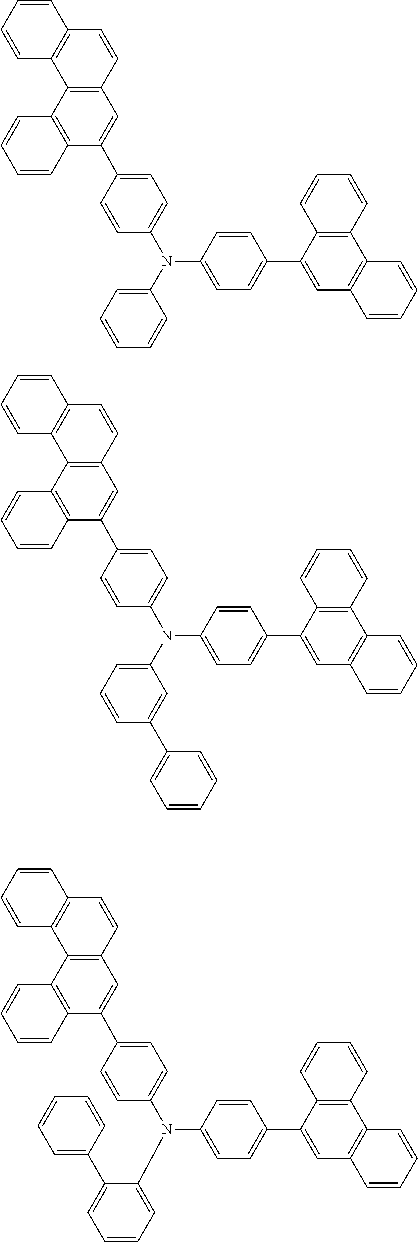 [SMILES: c1ccc(N(c2ccc(-c3cc4ccccc4c4ccccc34)cc2)c2ccc(-c3cc4ccc5ccccc5c4c4ccccc34)cc2)cc1 ; c1ccc(-c2cccc(N(c3ccc(-c4cc5ccccc5c5ccccc45)cc3)c3ccc(-c4cc5ccc6ccccc6c5c5ccccc45)cc3)c2)cc1 ; c1ccc(-c2ccccc2N(c2ccc(-c3cc4ccccc4c4ccccc34)cc2)c2ccc(-c3cc4ccc5ccccc5c4c4ccccc34)cc2)cc1]

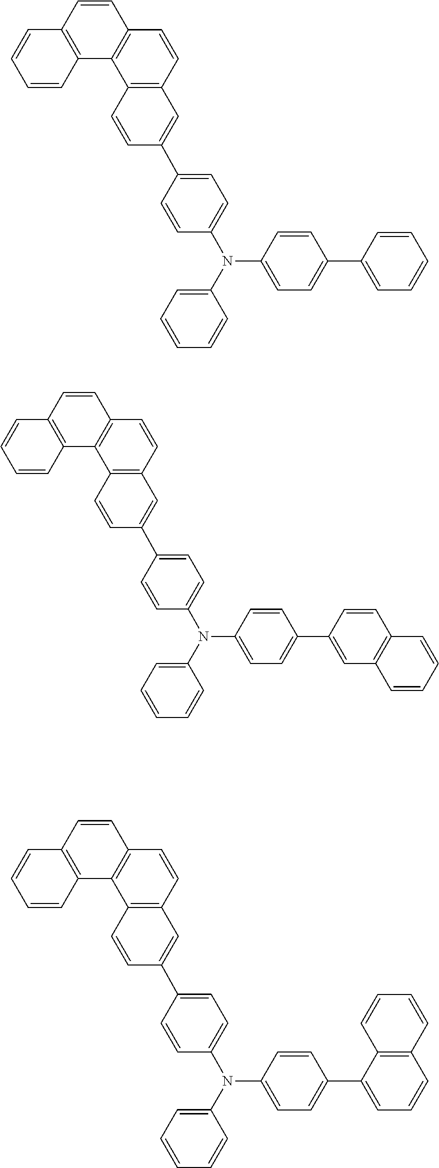 [SMILES: c1ccc(-c2ccc(N(c3ccccc3)c3ccc(-c4ccc5c(ccc6ccc7ccccc7c65)c4)cc3)cc2)cc1 ; c1ccc(N(c2ccc(-c3ccc4ccccc4c3)cc2)c2ccc(-c3ccc4c(ccc5ccc6ccccc6c54)c3)cc2)cc1 ; c1ccc(N(c2ccc(-c3ccc4c(ccc5ccc6ccccc6c54)c3)cc2)c2ccc(-c3cccc4ccccc34)cc2)cc1]

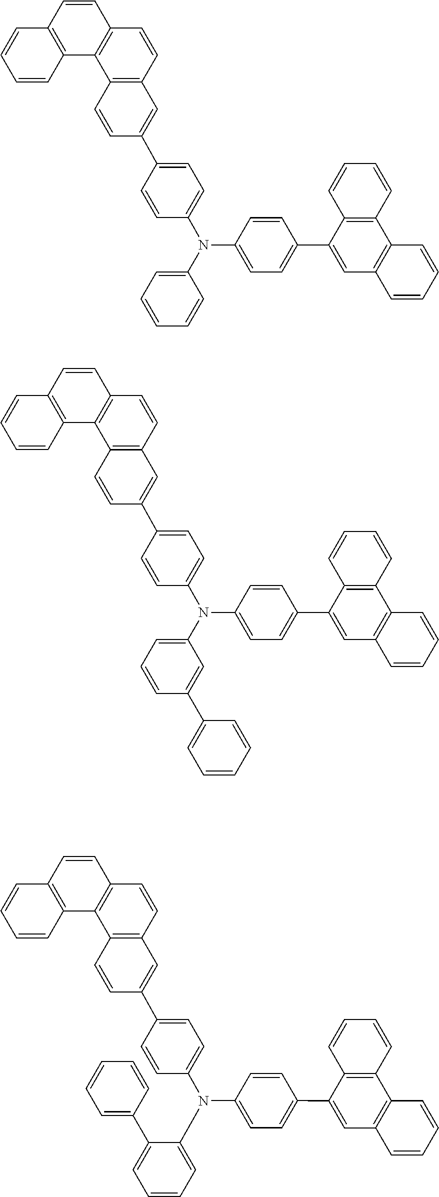 [SMILES: c1ccc(N(c2ccc(-c3ccc4c(ccc5ccc6ccccc6c54)c3)cc2)c2ccc(-c3cc4ccccc4c4ccccc34)cc2)cc1 ; c1ccc(-c2cccc(N(c3ccc(-c4ccc5c(ccc6ccc7ccccc7c65)c4)cc3)c3ccc(-c4cc5ccccc5c5ccccc45)cc3)c2)cc1 ; c1ccc(-c2ccccc2N(c2ccc(-c3ccc4c(ccc5ccc6ccccc6c54)c3)cc2)c2ccc(-c3cc4ccccc4c4ccccc34)cc2)cc1]

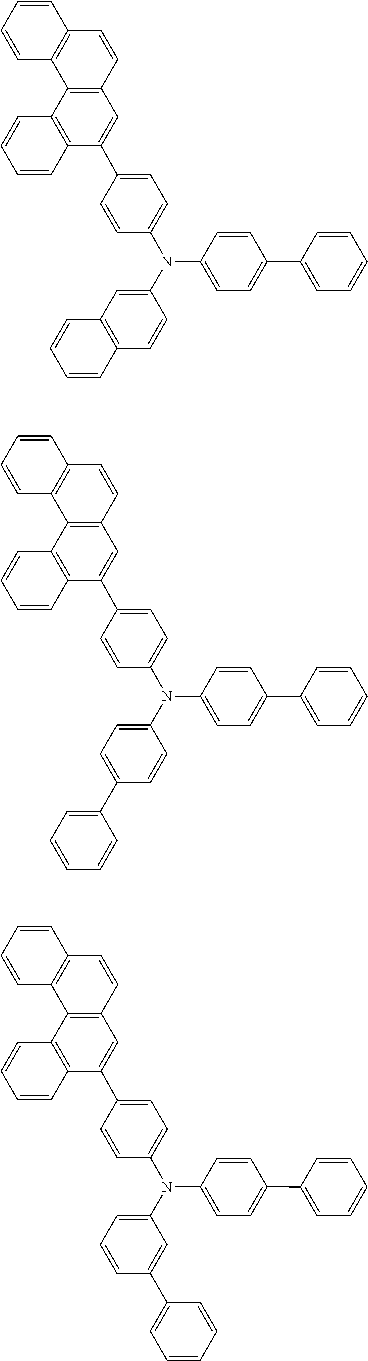 [SMILES: c1ccc(-c2ccc(N(c3ccc(-c4cc5ccc6ccccc6c5c5ccccc45)cc3)c3ccc4ccccc4c3)cc2)cc1 ; c1ccc(-c2ccc(N(c3ccc(-c4ccccc4)cc3)c3ccc(-c4cc5ccc6ccccc6c5c5ccccc45)cc3)cc2)cc1 ; c1ccc(-c2ccc(N(c3ccc(-c4cc5ccc6ccccc6c5c5ccccc45)cc3)c3cccc(-c4ccccc4)c3)cc2)cc1]

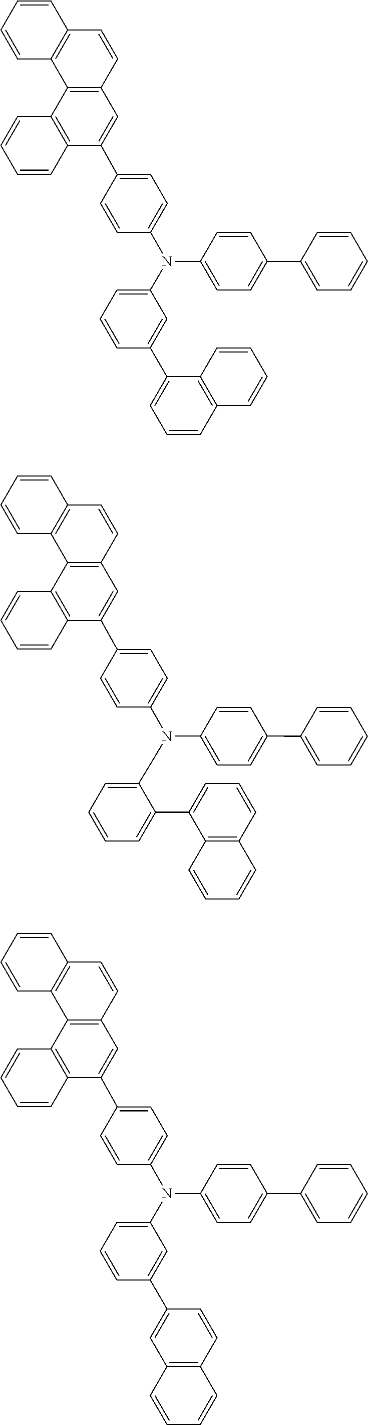 [SMILES: c1ccc(-c2ccc(N(c3ccc(-c4cc5ccc6ccccc6c5c5ccccc45)cc3)c3cccc(-c4cccc5ccccc45)c3)cc2)cc1 ; c1ccc(-c2ccc(N(c3ccc(-c4cc5ccc6ccccc6c5c5ccccc45)cc3)c3ccccc3-c3cccc4ccccc34)cc2)cc1 ; c1ccc(-c2ccc(N(c3ccc(-c4cc5ccc6ccccc6c5c5ccccc45)cc3)c3cccc(-c4ccc5ccccc5c4)c3)cc2)cc1]

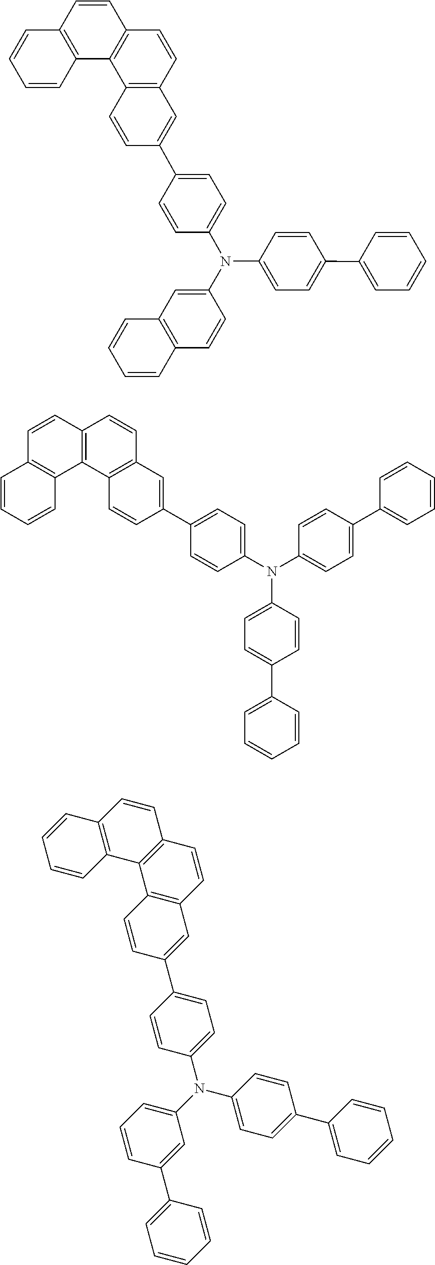

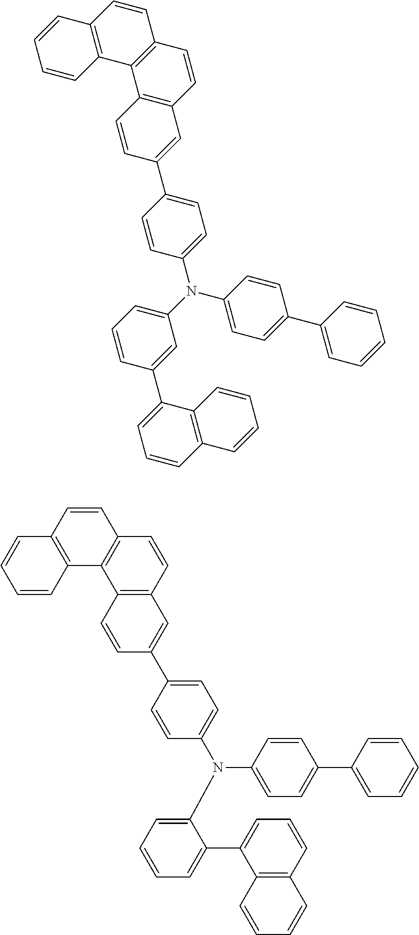 [SMILES: c1ccc(-c2ccc(N(c3ccc(-c4ccc5c(ccc6ccc7ccccc7c65)c4)cc3)c3cccc(-c4cccc5ccccc45)c3)cc2)cc1 ; c1ccc(-c2ccc(N(c3ccc(-c4ccc5c(ccc6ccc7ccccc7c65)c4)cc3)c3ccccc3-c3cccc4ccccc34)cc2)cc1]

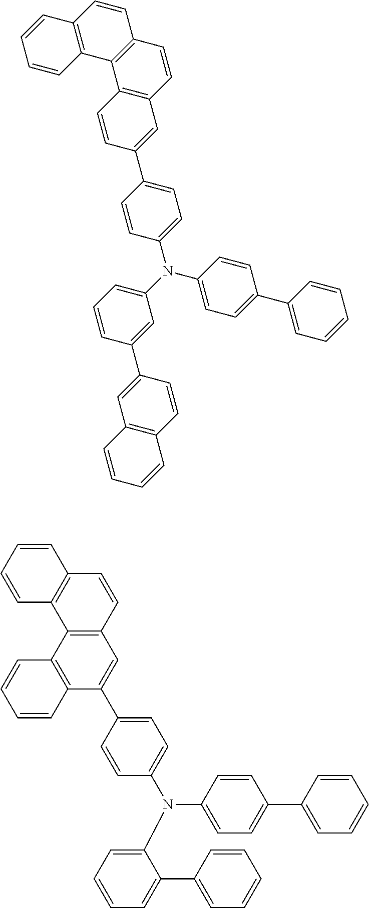 [SMILES: c1ccc(-c2ccc(N(c3ccc(-c4ccc5c(ccc6ccc7ccccc7c65)c4)cc3)c3cccc(-c4ccc5ccccc5c4)c3)cc2)cc1 ; c1ccc(-c2ccc(N(c3ccc(-c4cc5ccc6ccccc6c5c5ccccc45)cc3)c3ccccc3-c3ccccc3)cc2)cc1]

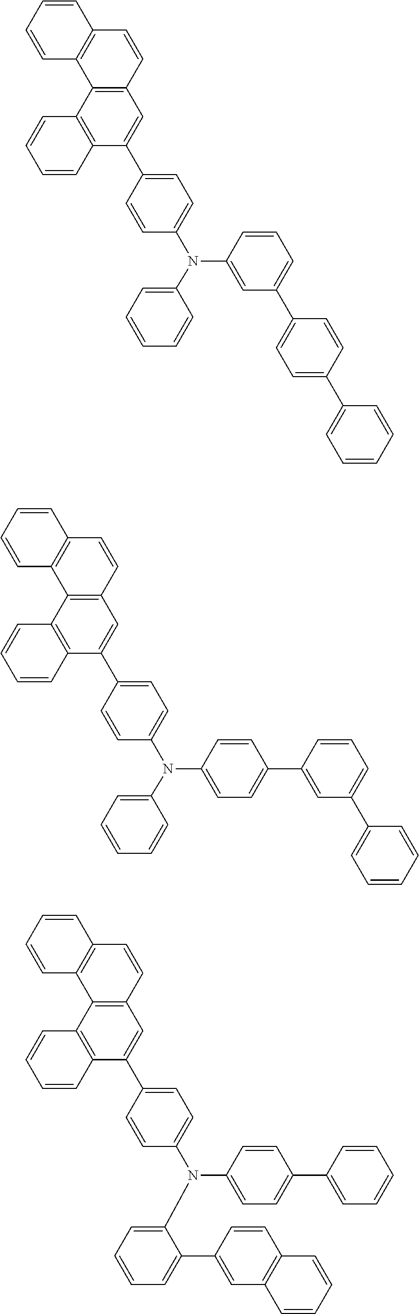 [SMILES: c1ccc(-c2ccc(-c3cccc(N(c4ccccc4)c4ccc(-c5cc6ccc7ccccc7c6c6ccccc56)cc4)c3)cc2)cc1 ; c1ccc(-c2cccc(-c3ccc(N(c4ccccc4)c4ccc(-c5cc6ccc7ccccc7c6c6ccccc56)cc4)cc3)c2)cc1 ; c1ccc(-c2ccc(N(c3ccc(-c4cc5ccc6ccccc6c5c5ccccc45)cc3)c3ccccc3-c3ccc4ccccc4c3)cc2)cc1]

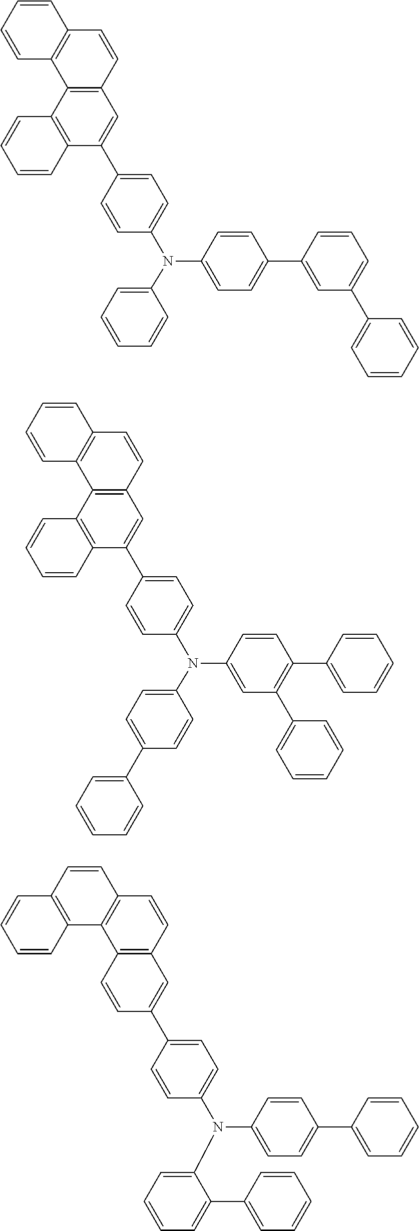

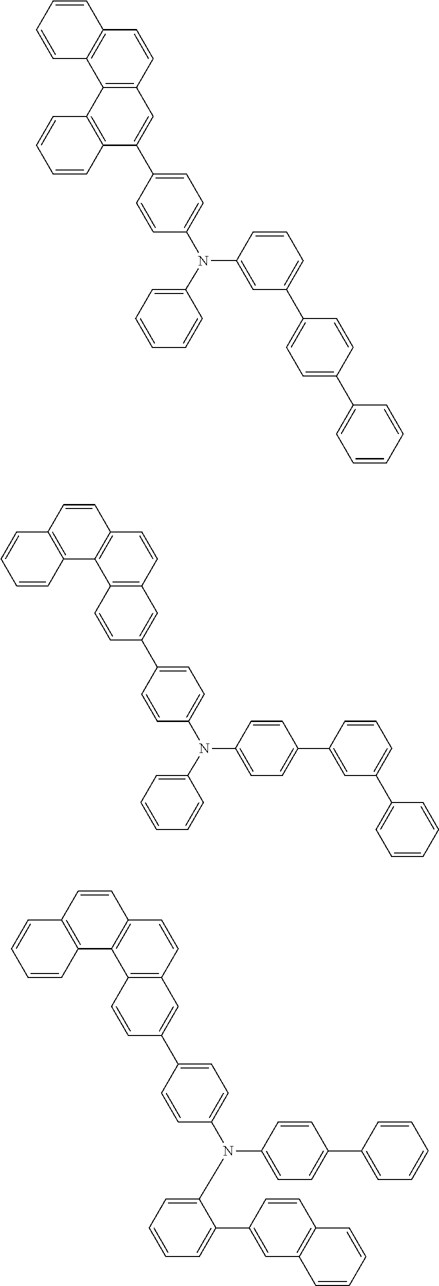 [SMILES: c1ccc(-c2ccc(-c3cccc(N(c4ccccc4)c4ccc(-c5cc6ccc7ccccc7c6c6ccccc56)cc4)c3)cc2)cc1 ; c1ccc(-c2cccc(-c3ccc(N(c4ccccc4)c4ccc(-c5ccc6c(ccc7ccc8ccccc8c76)c5)cc4)cc3)c2)cc1 ; c1ccc(-c2ccc(N(c3ccc(-c4ccc5c(ccc6ccc7ccccc7c65)c4)cc3)c3ccccc3-c3ccc4ccccc4c3)cc2)cc1]

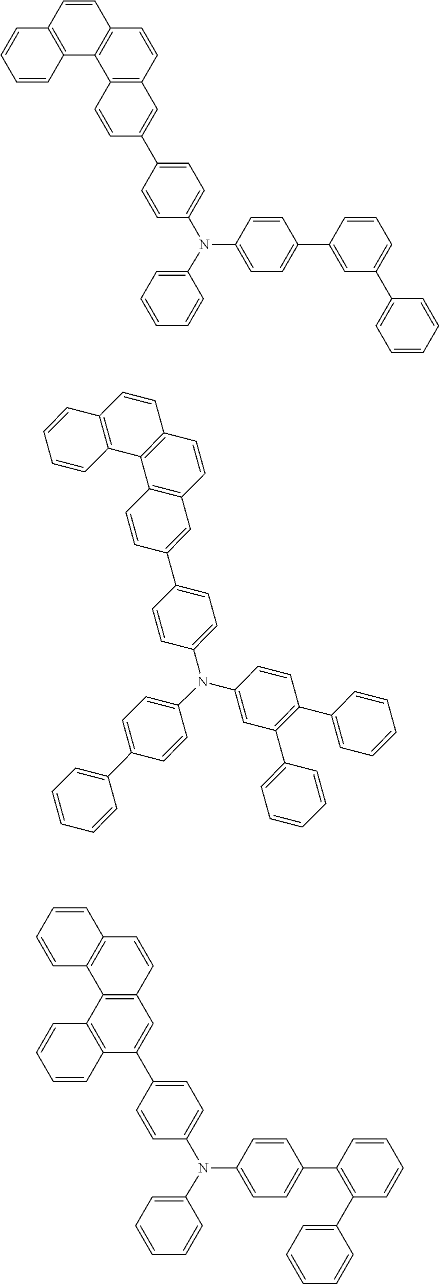 [SMILES: c1ccc(-c2cccc(-c3ccc(N(c4ccccc4)c4ccc(-c5ccc6c(ccc7ccc8ccccc8c76)c5)cc4)cc3)c2)cc1 ; c1ccc(-c2ccc(N(c3ccc(-c4ccc5c(ccc6ccc7ccccc7c65)c4)cc3)c3ccc(-c4ccccc4)c(-c4ccccc4)c3)cc2)cc1 ; c1ccc(-c2ccccc2-c2ccc(N(c3ccccc3)c3ccc(-c4cc5ccc6ccccc6c5c5ccccc45)cc3)cc2)cc1]

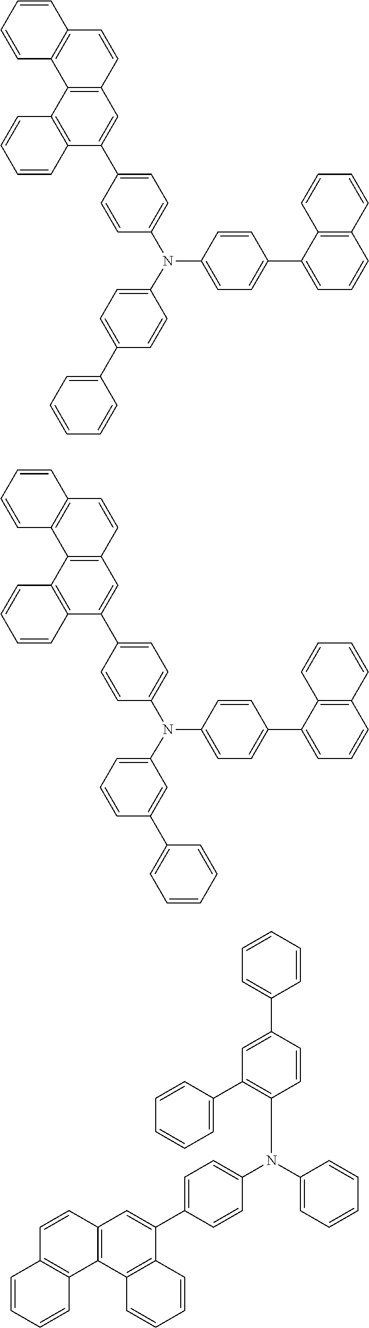

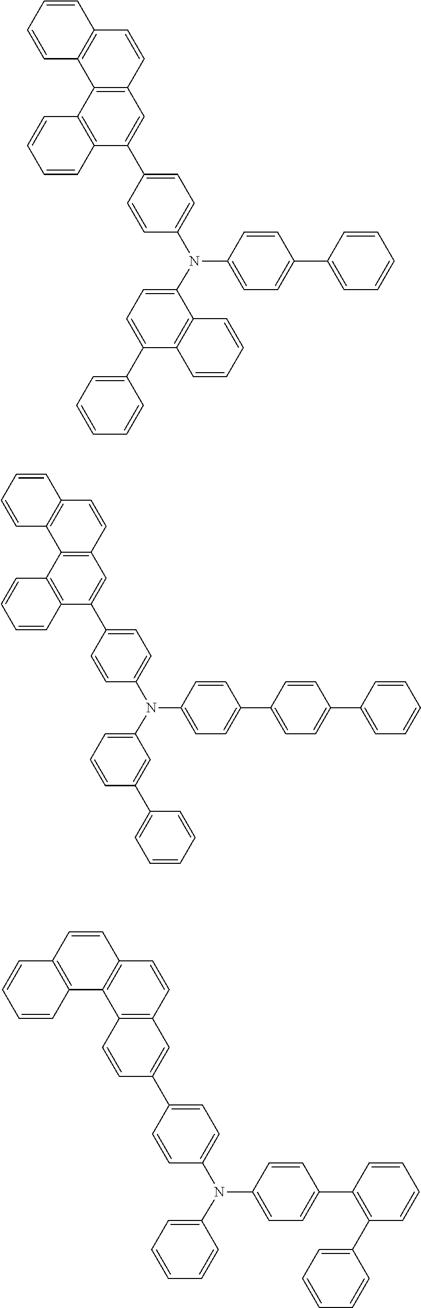 [SMILES: c1ccc(-c2ccc(N(c3ccc(-c4cc5ccc6ccccc6c5c5ccccc45)cc3)c3ccc(-c4ccccc4)c4ccccc34)cc2)cc1 ; c1ccc(-c2ccc(-c3ccc(N(c4ccc(-c5cc6ccc7ccccc7c6c6ccccc56)cc4)c4cccc(-c5ccccc5)c4)cc3)cc2)cc1 ; c1ccc(-c2ccccc2-c2ccc(N(c3ccccc3)c3ccc(-c4ccc5c(ccc6ccc7ccccc7c65)c4)cc3)cc2)cc1]

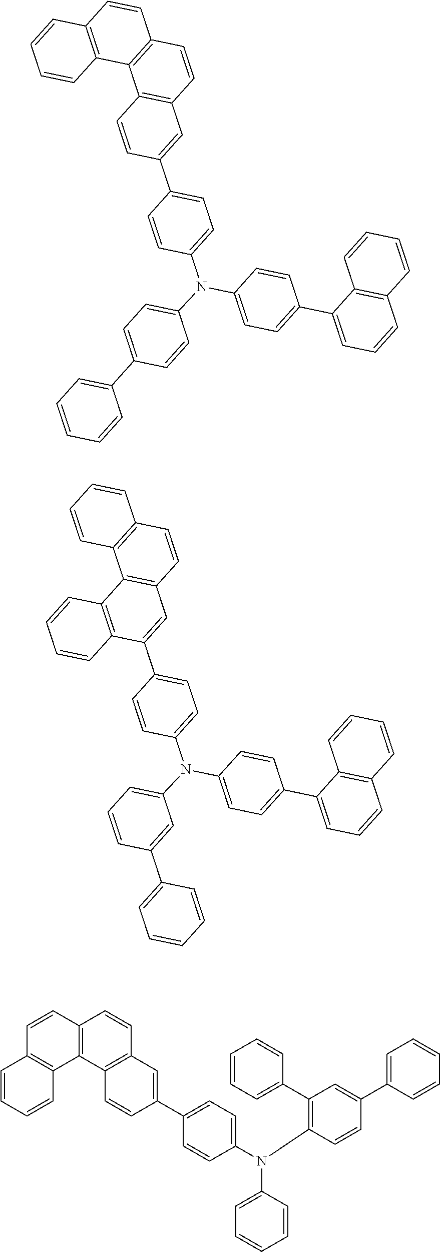

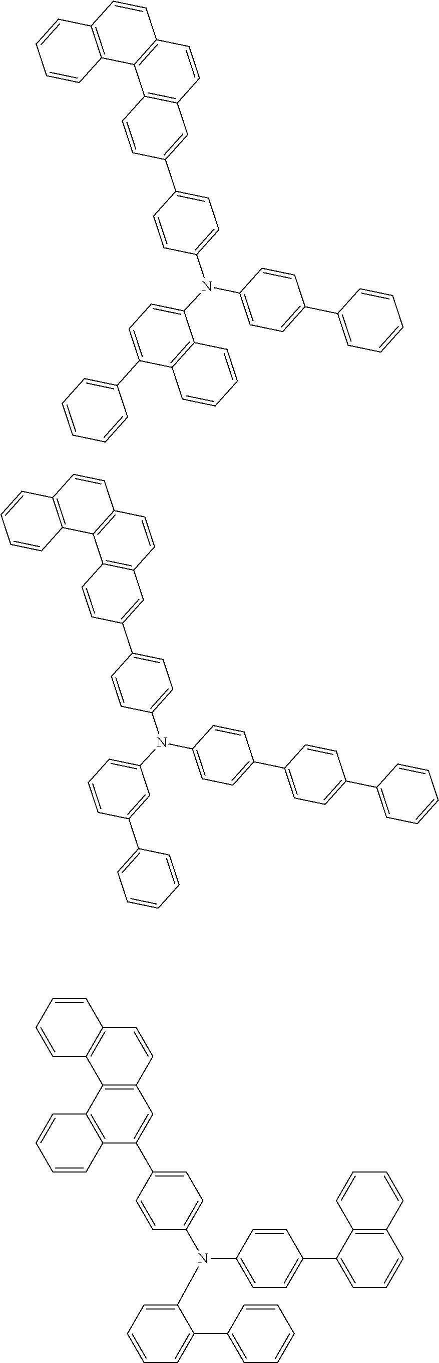

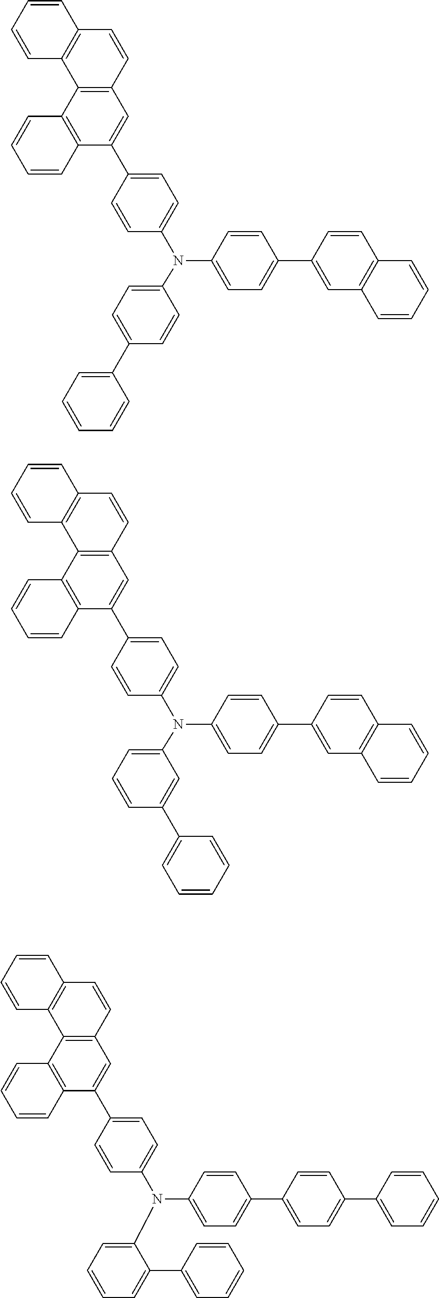

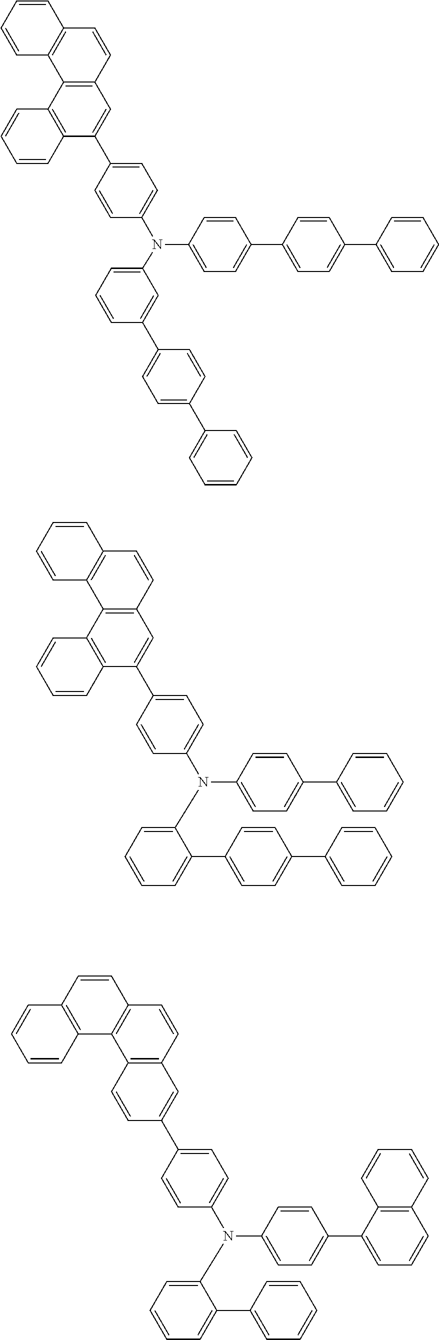 [SMILES: c1ccc(-c2ccc(-c3ccc(N(c4ccc(-c5cc6ccc7ccccc7c6c6ccccc56)cc4)c4cccc(-c5ccc(-c6ccccc6)cc5)c4)cc3)cc2)cc1 ; c1ccc(-c2ccc(-c3ccccc3N(c3ccc(-c4ccccc4)cc3)c3ccc(-c4cc5ccc6ccccc6c5c5ccccc45)cc3)cc2)cc1 ; c1ccc(-c2ccccc2N(c2ccc(-c3ccc4c(ccc5ccc6ccccc6c54)c3)cc2)c2ccc(-c3cccc4ccccc34)cc2)cc1]

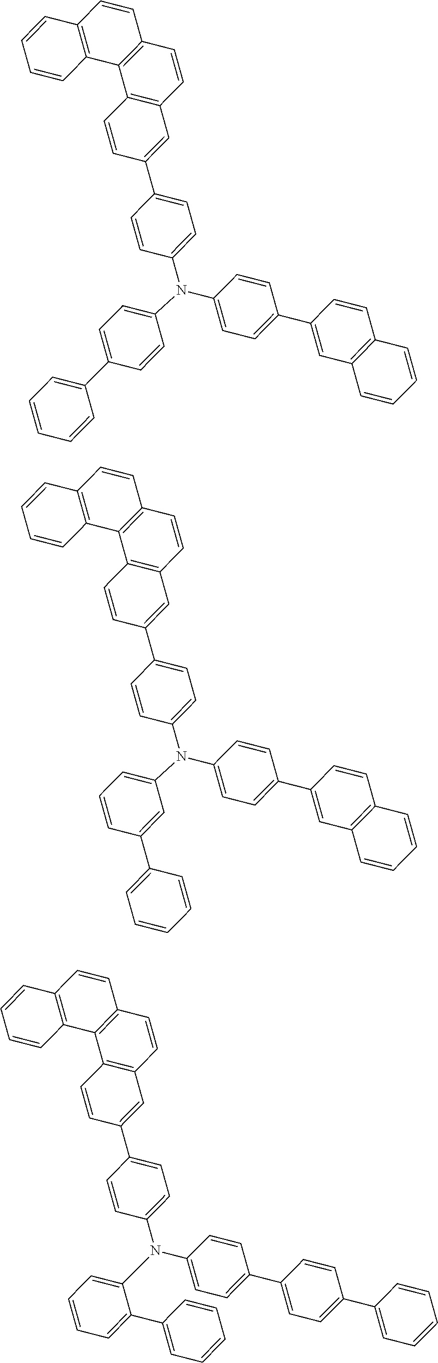

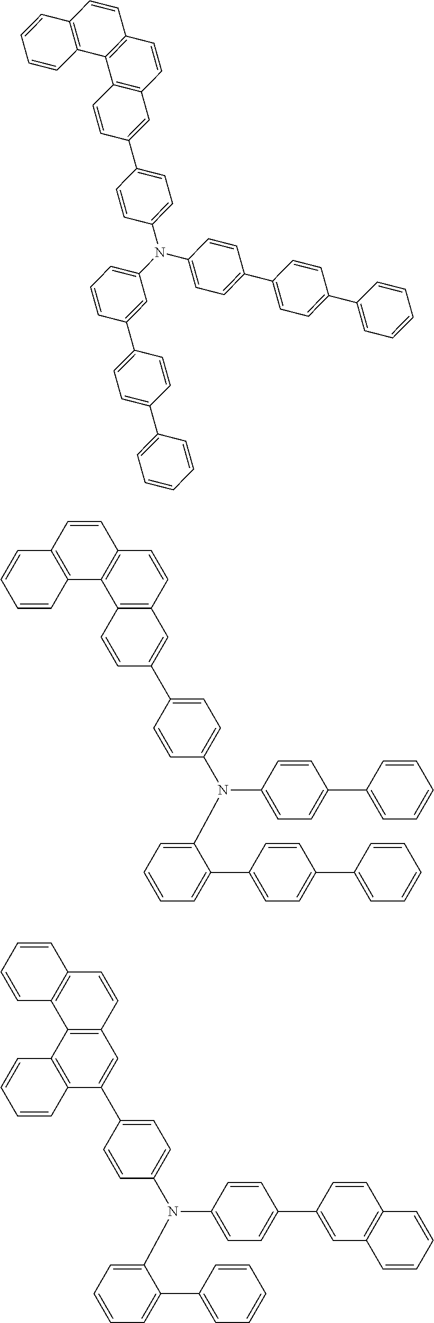 [SMILES: c1ccc(-c2ccc(-c3ccc(N(c4ccc(-c5ccc6c(ccc7ccc8ccccc8c76)c5)cc4)c4cccc(-c5ccc(-c6ccccc6)cc5)c4)cc3)cc2)cc1 ; c1ccc(-c2ccc(-c3ccccc3N(c3ccc(-c4ccccc4)cc3)c3ccc(-c4ccc5c(ccc6ccc7ccccc7c65)c4)cc3)cc2)cc1 ; c1ccc(-c2ccccc2N(c2ccc(-c3ccc4ccccc4c3)cc2)c2ccc(-c3cc4ccc5ccccc5c4c4ccccc34)cc2)cc1]

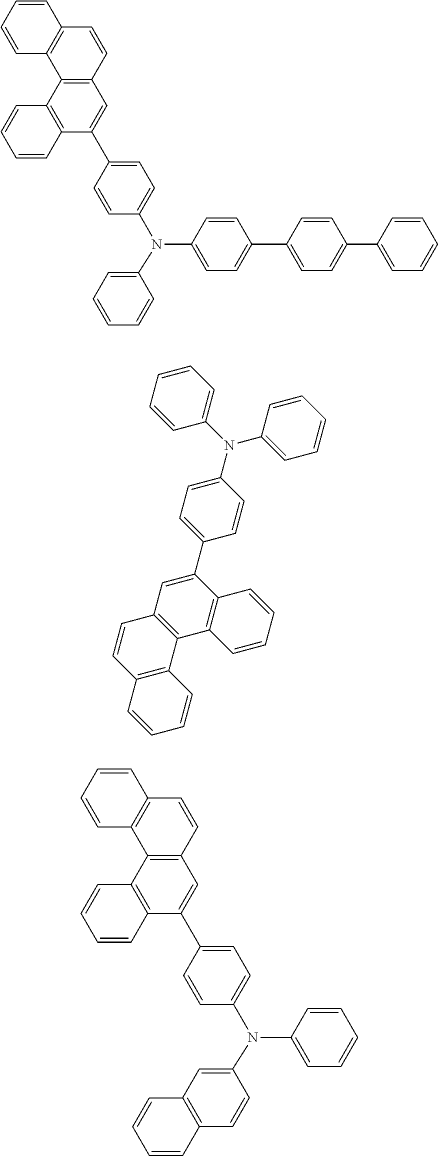

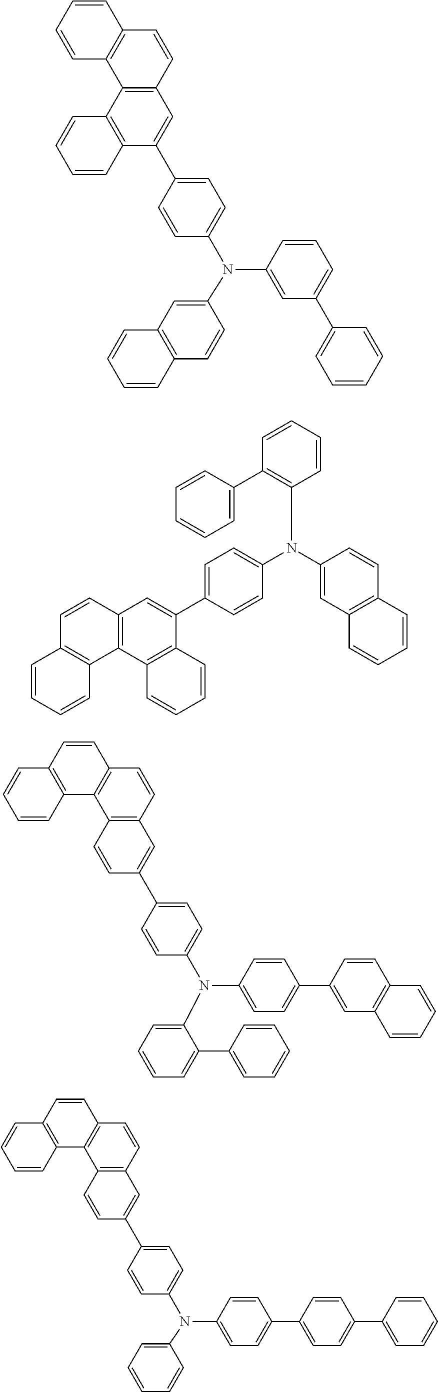

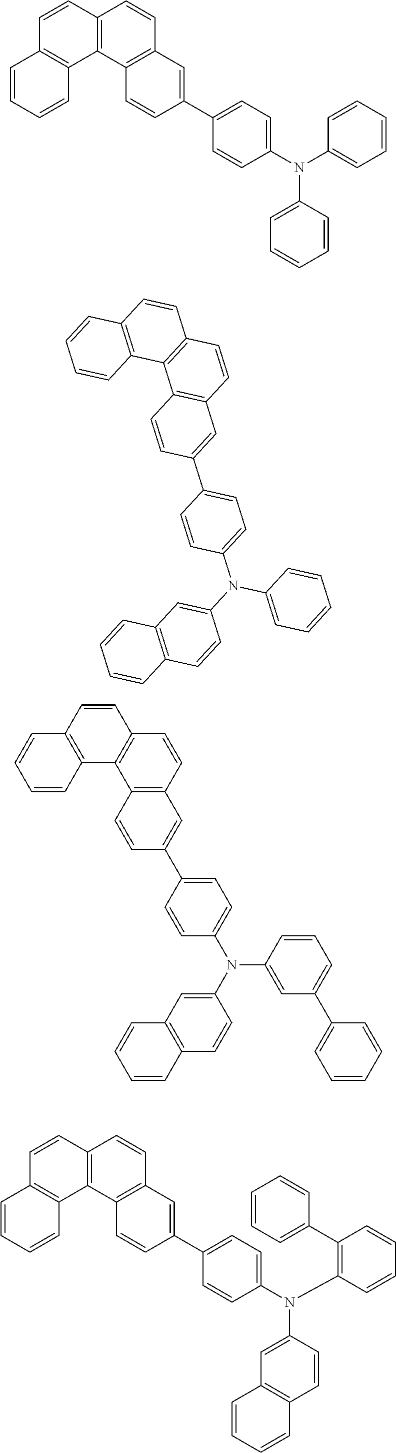

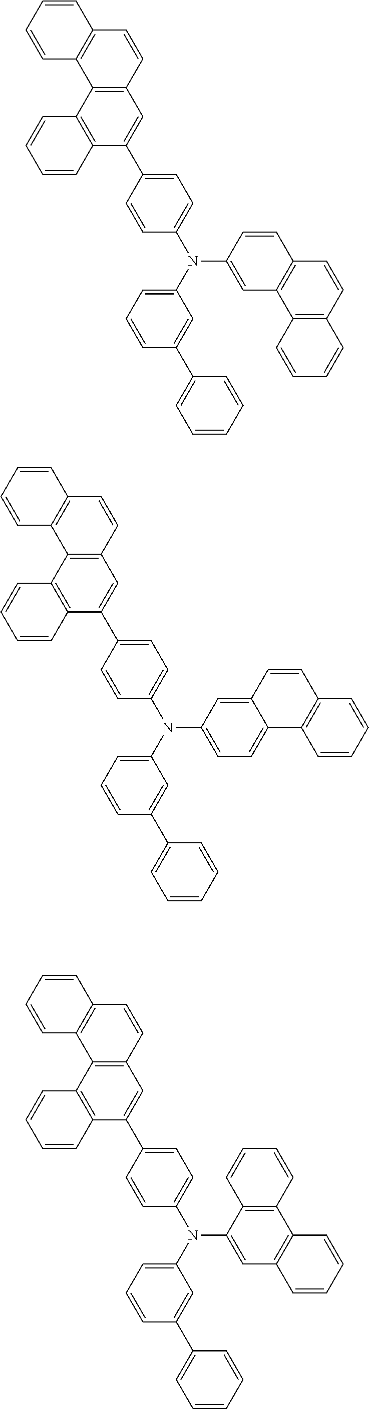 [SMILES: c1ccc(-c2cccc(N(c3ccc(-c4cc5ccc6ccccc6c5c5ccccc45)cc3)c3ccc4ccc5ccccc5c4c3)c2)cc1 ; c1ccc(-c2cccc(N(c3ccc(-c4cc5ccc6ccccc6c5c5ccccc45)cc3)c3ccc4c(ccc5ccccc54)c3)c2)cc1 ; c1ccc(-c2cccc(N(c3ccc(-c4cc5ccc6ccccc6c5c5ccccc45)cc3)c3cc4ccccc4c4ccccc34)c2)cc1]

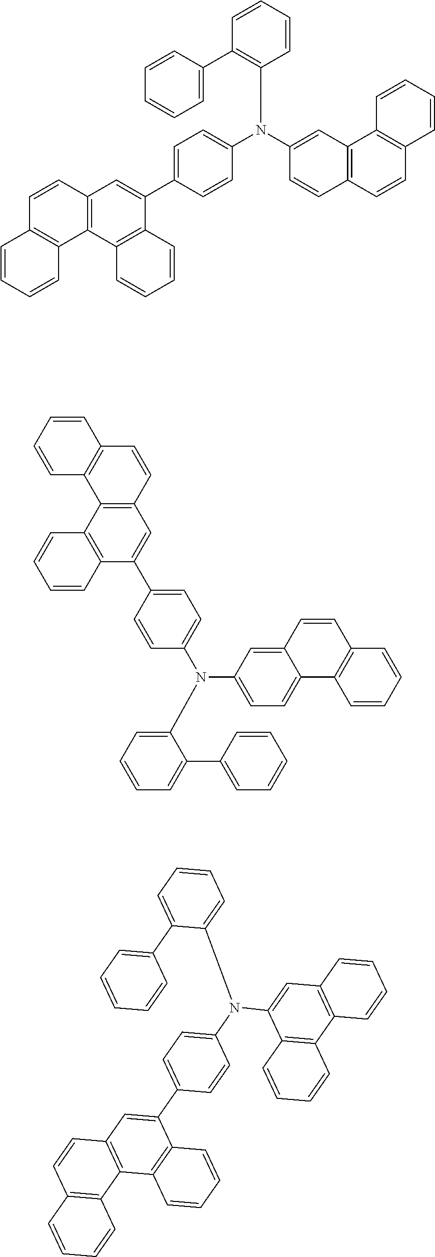 [SMILES: c1ccc(-c2ccccc2N(c2ccc(-c3cc4ccc5ccccc5c4c4ccccc34)cc2)c2ccc3ccc4ccccc4c3c2)cc1 ; c1ccc(-c2ccccc2N(c2ccc(-c3cc4ccc5ccccc5c4c4ccccc34)cc2)c2ccc3c(ccc4ccccc43)c2)cc1 ; c1ccc(-c2ccccc2N(c2ccc(-c3cc4ccc5ccccc5c4c4ccccc34)cc2)c2cc3ccccc3c3ccccc23)cc1]

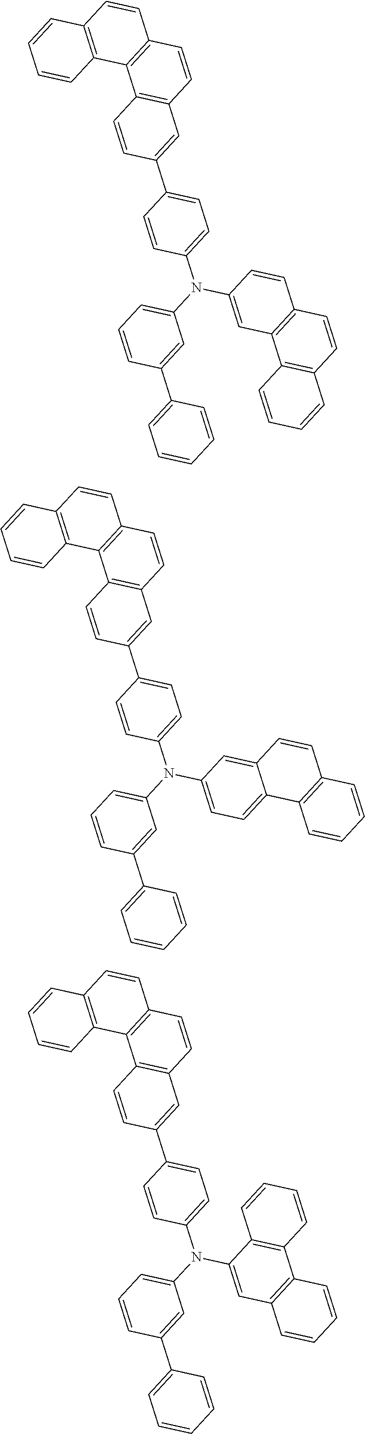

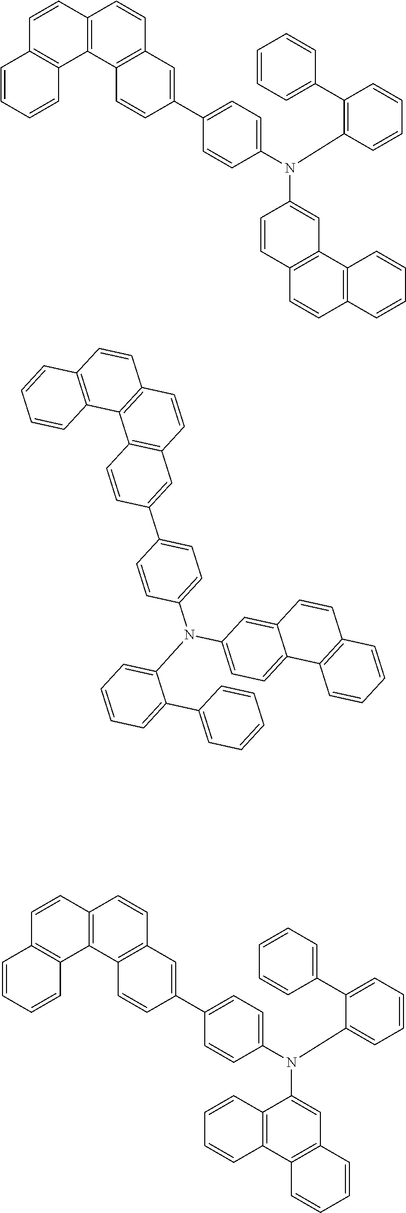 [SMILES: c1ccc(-c2ccccc2N(c2ccc(-c3ccc4c(ccc5ccc6ccccc6c54)c3)cc2)c2ccc3ccc4ccccc4c3c2)cc1 ; c1ccc(-c2ccccc2N(c2ccc(-c3ccc4c(ccc5ccc6ccccc6c54)c3)cc2)c2ccc3c(ccc4ccccc43)c2)cc1 ; c1ccc(-c2ccccc2N(c2ccc(-c3ccc4c(ccc5ccc6ccccc6c54)c3)cc2)c2cc3ccccc3c3ccccc23)cc1]

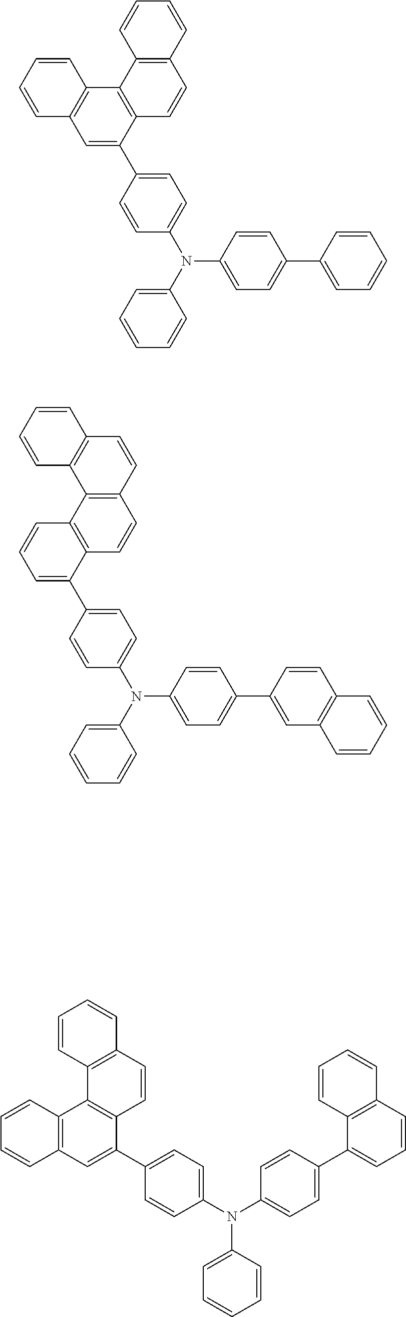 [SMILES: c1ccc(-c2ccc(N(c3ccccc3)c3ccc(-c4cc5ccccc5c5c4ccc4ccccc45)cc3)cc2)cc1 ; c1ccc(N(c2ccc(-c3ccc4ccccc4c3)cc2)c2ccc(-c3cccc4c3ccc3ccc5ccccc5c34)cc2)cc1 ; c1ccc(N(c2ccc(-c3cccc4ccccc34)cc2)c2ccc(-c3cc4ccccc4c4c3ccc3ccccc34)cc2)cc1]

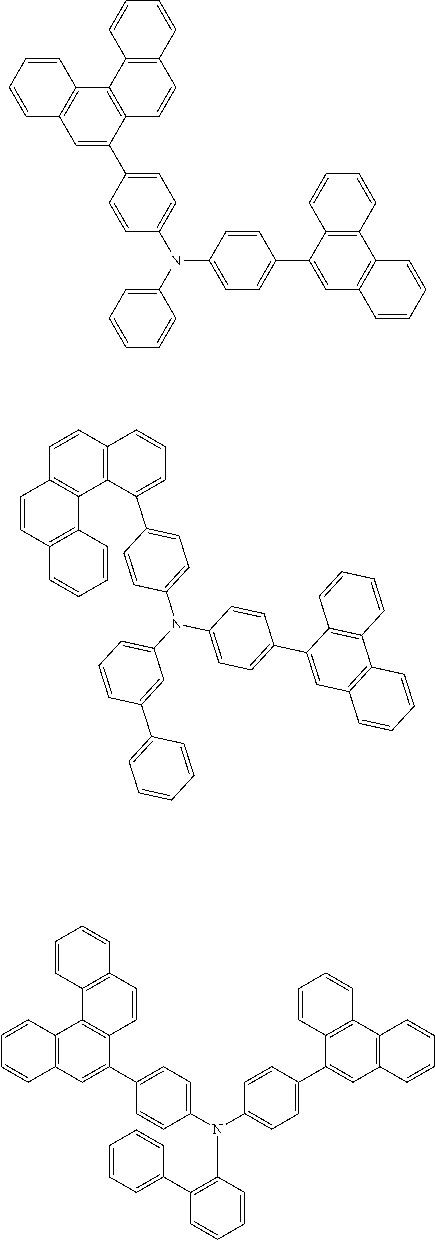 [SMILES: c1ccc(N(c2ccc(-c3cc4ccccc4c4ccccc34)cc2)c2ccc(-c3cc4ccccc4c4c3ccc3ccccc34)cc2)cc1 ; c1ccc(-c2cccc(N(c3ccc(-c4cc5ccccc5c5ccccc45)cc3)c3ccc(-c4cccc5ccc6ccc7ccccc7c6c45)cc3)c2)cc1 ; c1ccc(-c2ccccc2N(c2ccc(-c3cc4ccccc4c4ccccc34)cc2)c2ccc(-c3cc4ccccc4c4c3ccc3ccccc34)cc2)cc1]

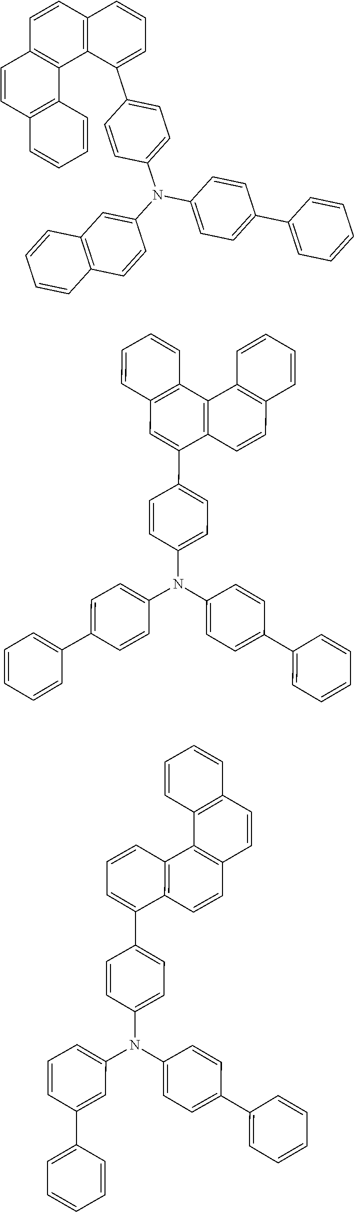 [SMILES: c1ccc(-c2ccc(N(c3ccc(-c4cccc5ccc6ccc7ccccc7c6c45)cc3)c3ccc4ccccc4c3)cc2)cc1 ; c1ccc(-c2ccc(N(c3ccc(-c4ccccc4)cc3)c3ccc(-c4cc5ccccc5c5c4ccc4ccccc45)cc3)cc2)cc1 ; c1ccc(-c2ccc(N(c3ccc(-c4cccc5c4ccc4ccc6ccccc6c45)cc3)c3cccc(-c4ccccc4)c3)cc2)cc1]

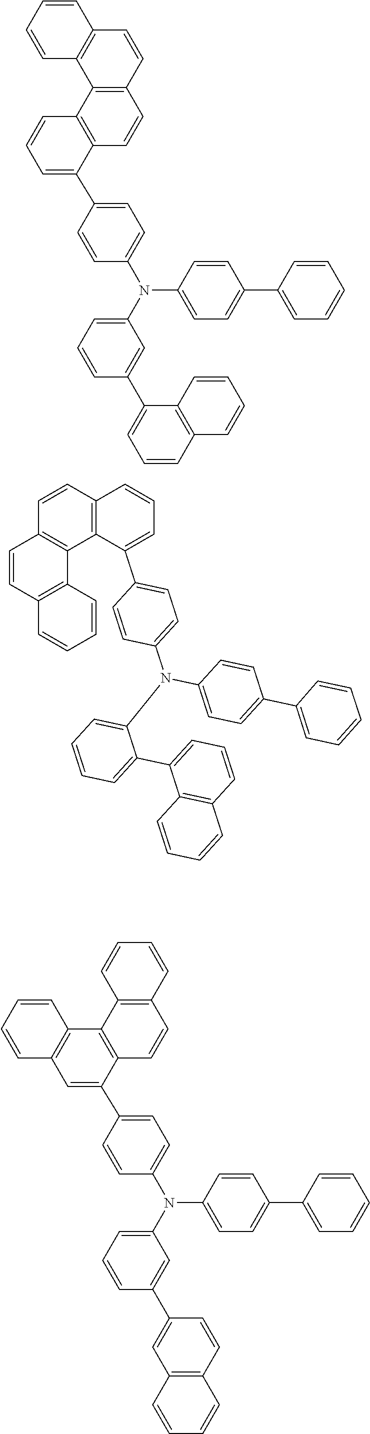

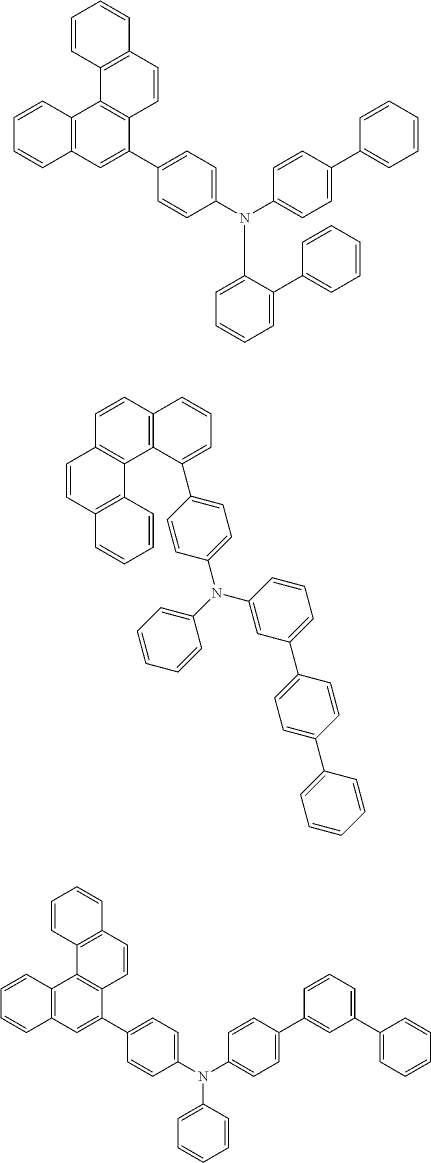 [SMILES: c1ccc(-c2ccc(N(c3ccc(-c4cc5ccccc5c5c4ccc4ccccc45)cc3)c3ccccc3-c3ccccc3)cc2)cc1 ; c1ccc(-c2ccc(-c3cccc(N(c4ccccc4)c4ccc(-c5cccc6ccc7ccc8ccccc8c7c56)cc4)c3)cc2)cc1 ; c1ccc(-c2cccc(-c3ccc(N(c4ccccc4)c4ccc(-c5cc6ccccc6c6c5ccc5ccccc56)cc4)cc3)c2)cc1]

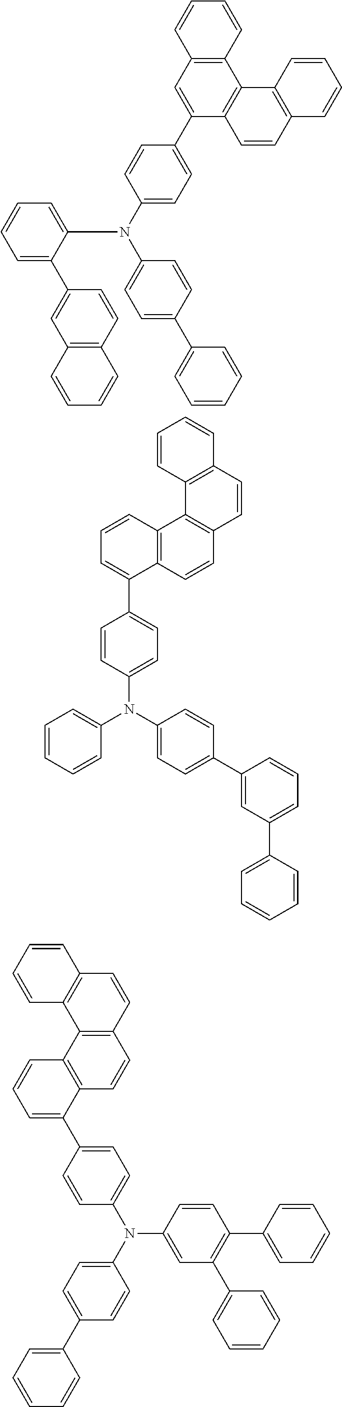

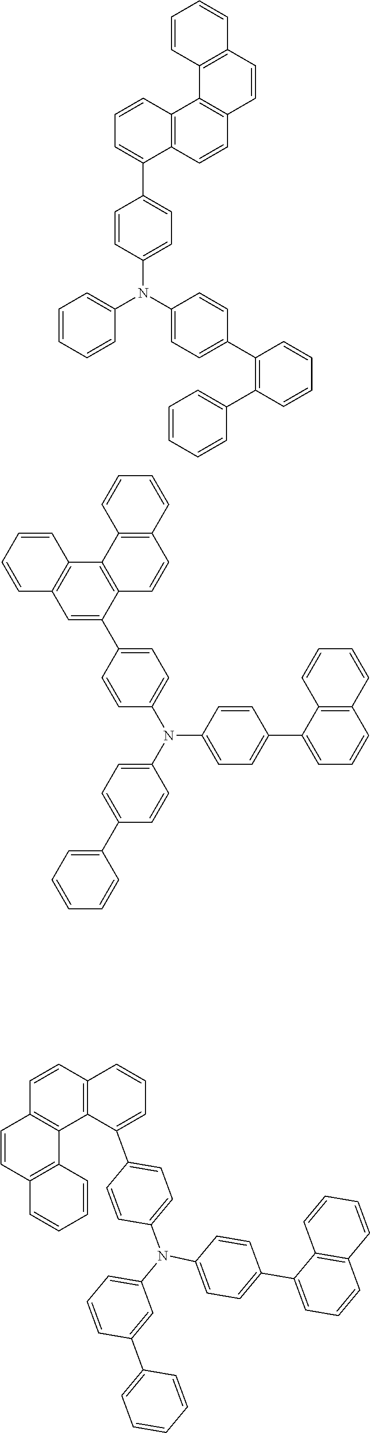

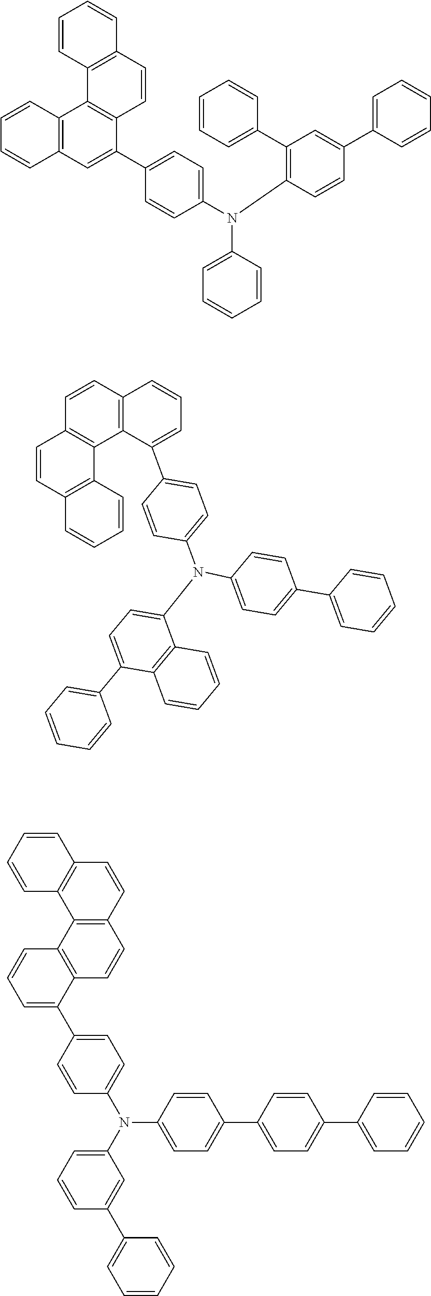 [SMILES: c1ccc(-c2ccc(N(c3ccccc3)c3ccc(-c4cc5ccccc5c5c4ccc4ccccc45)cc3)c(-c3ccccc3)c2)cc1 ; c1ccc(-c2ccc(N(c3ccc(-c4cccc5ccc6ccc7ccccc7c6c45)cc3)c3ccc(-c4ccccc4)c4ccccc34)cc2)cc1 ; c1ccc(-c2ccc(-c3ccc(N(c4ccc(-c5cccc6c5ccc5ccc7ccccc7c56)cc4)c4cccc(-c5ccccc5)c4)cc3)cc2)cc1]

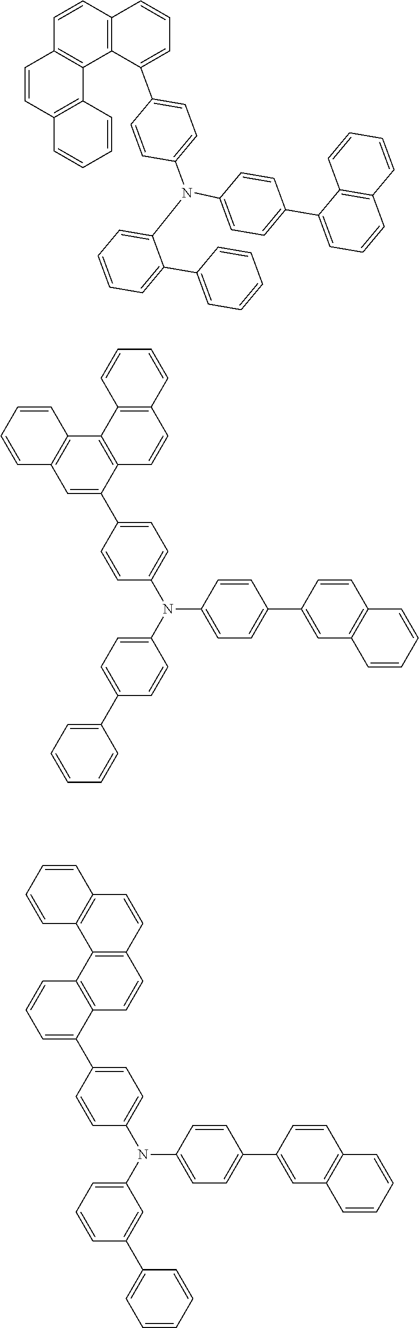 [SMILES: c1ccc(-c2ccccc2N(c2ccc(-c3cccc4ccccc34)cc2)c2ccc(-c3cccc4ccc5ccc6ccccc6c5c34)cc2)cc1 ; c1ccc(-c2ccc(N(c3ccc(-c4ccc5ccccc5c4)cc3)c3ccc(-c4cc5ccccc5c5c4ccc4ccccc45)cc3)cc2)cc1 ; c1ccc(-c2cccc(N(c3ccc(-c4ccc5ccccc5c4)cc3)c3ccc(-c4cccc5c4ccc4ccc6ccccc6c45)cc3)c2)cc1]

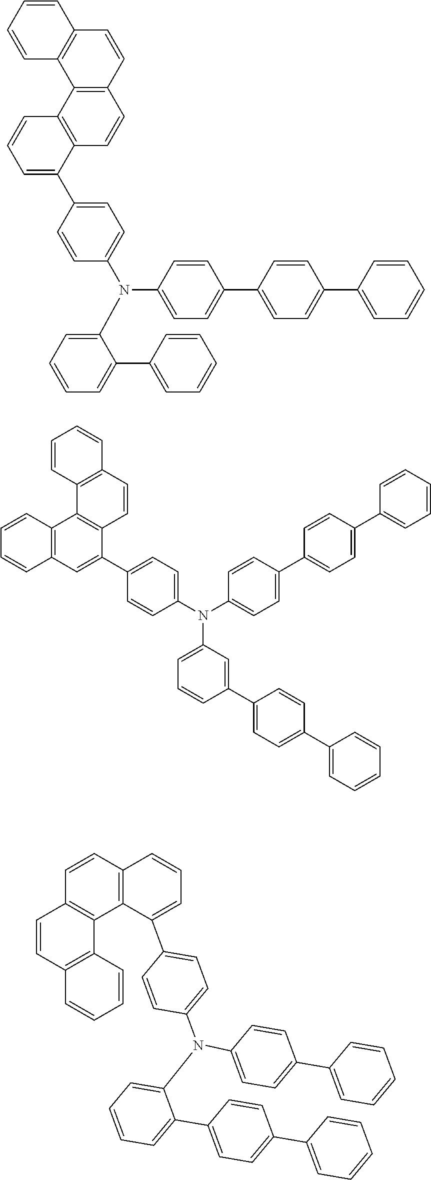

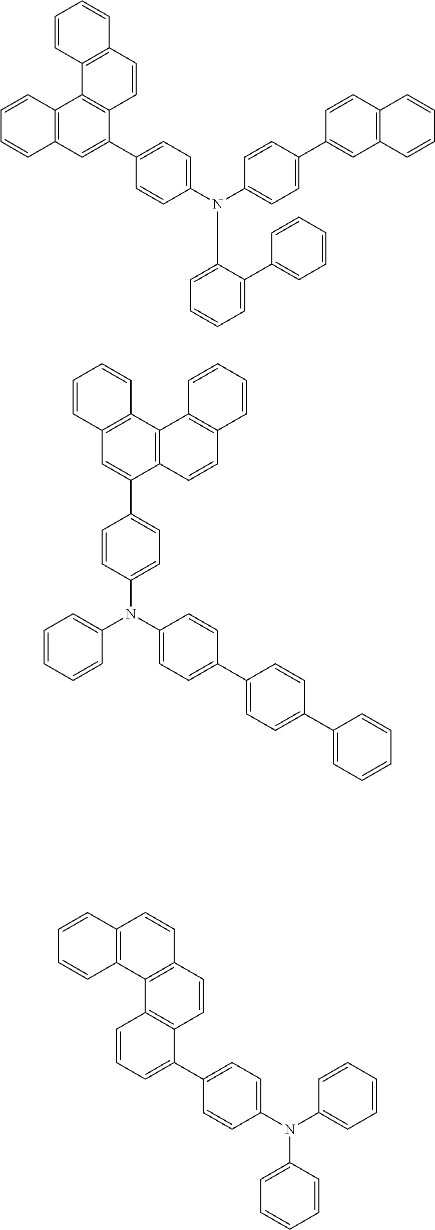

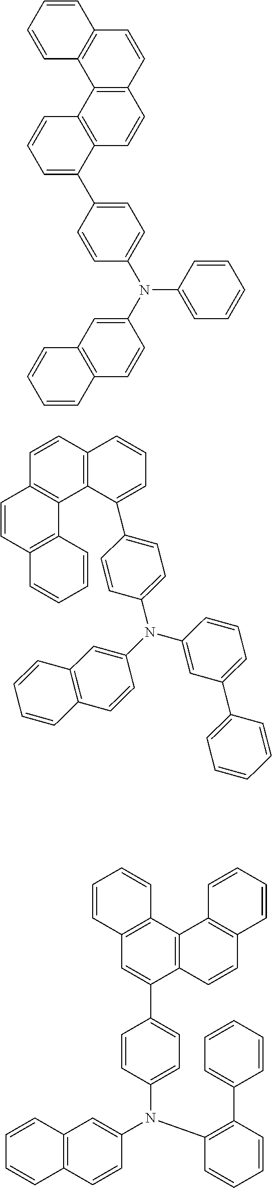

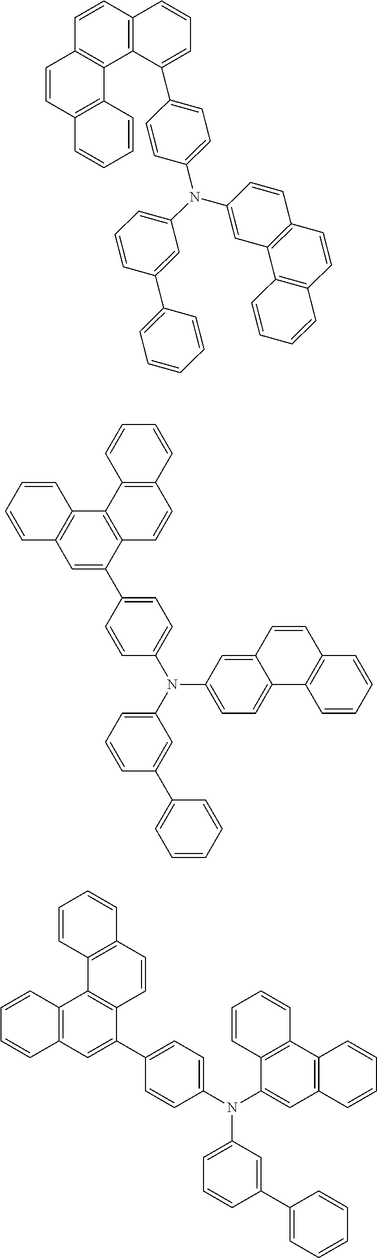 [SMILES: c1ccc(-c2cccc(N(c3ccc(-c4cccc5ccc6ccc7ccccc7c6c45)cc3)c3ccc4ccc5ccccc5c4c3)c2)cc1 ; c1ccc(-c2cccc(N(c3ccc(-c4cc5ccccc5c5c4ccc4ccccc45)cc3)c3ccc4c(ccc5ccccc54)c3)c2)cc1 ; c1ccc(-c2cccc(N(c3ccc(-c4cc5ccccc5c5c4ccc4ccccc45)cc3)c3cc4ccccc4c4ccccc34)c2)cc1]

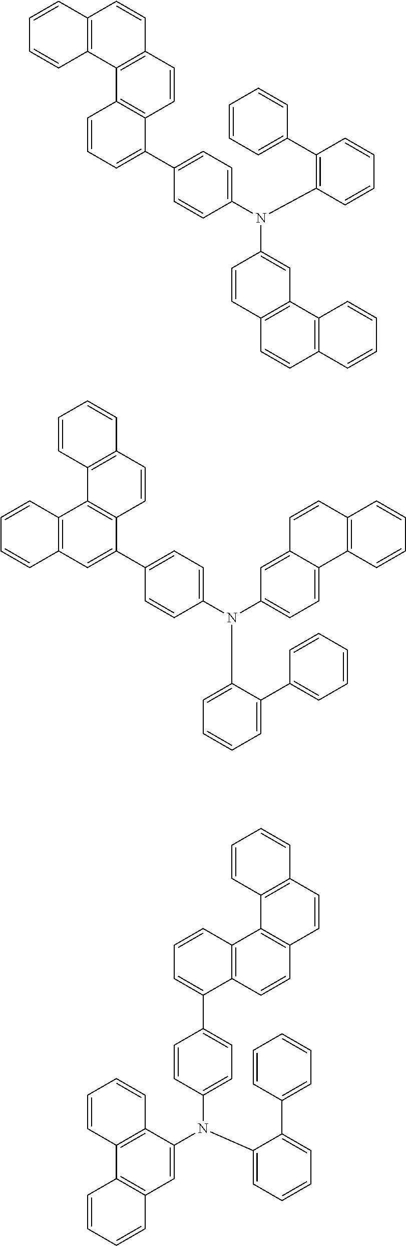 [SMILES: c1ccc(-c2ccccc2N(c2ccc(-c3cccc4c3ccc3ccc5ccccc5c34)cc2)c2ccc3ccc4ccccc4c3c2)cc1 ; c1ccc(-c2ccccc2N(c2ccc(-c3cc4ccccc4c4c3ccc3ccccc34)cc2)c2ccc3c(ccc4ccccc43)c2)cc1 ; c1ccc(-c2ccccc2N(c2ccc(-c3cccc4c3ccc3ccc5ccccc5c34)cc2)c2cc3ccccc3c3ccccc23)cc1]

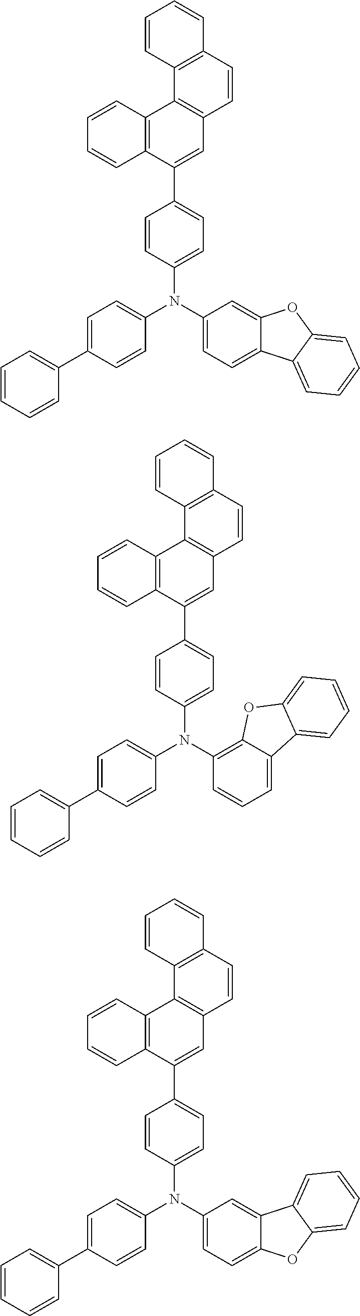 [SMILES: c1ccc(-c2ccc(N(c3ccc(-c4cc5ccc6ccccc6c5c5ccccc45)cc3)c3ccc4c(c3)oc3ccccc34)cc2)cc1 ; c1ccc(-c2ccc(N(c3ccc(-c4cc5ccc6ccccc6c5c5ccccc45)cc3)c3cccc4c3oc3ccccc34)cc2)cc1 ; c1ccc(-c2ccc(N(c3ccc(-c4cc5ccc6ccccc6c5c5ccccc45)cc3)c3ccc4oc5ccccc5c4c3)cc2)cc1]

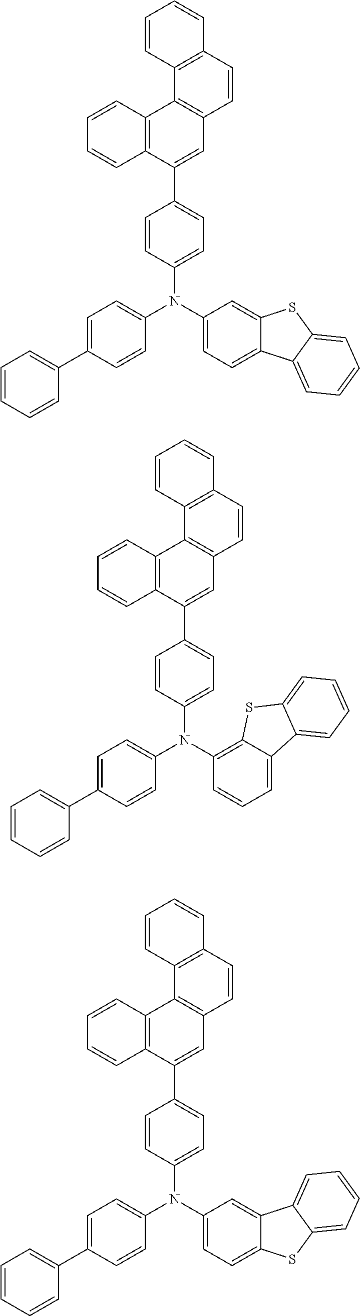 [SMILES: c1ccc(-c2ccc(N(c3ccc(-c4cc5ccc6ccccc6c5c5ccccc45)cc3)c3ccc4c(c3)sc3ccccc34)cc2)cc1 ; c1ccc(-c2ccc(N(c3ccc(-c4cc5ccc6ccccc6c5c5ccccc45)cc3)c3cccc4c3sc3ccccc34)cc2)cc1 ; c1ccc(-c2ccc(N(c3ccc(-c4cc5ccc6ccccc6c5c5ccccc45)cc3)c3ccc4sc5ccccc5c4c3)cc2)cc1]

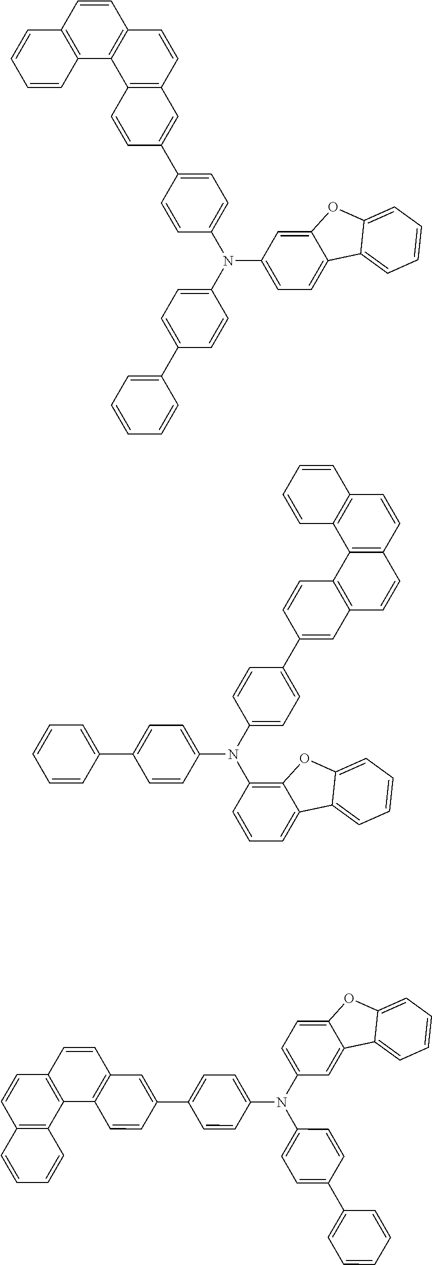 [SMILES: c1ccc(-c2ccc(N(c3ccc(-c4ccc5c(ccc6ccc7ccccc7c65)c4)cc3)c3ccc4c(c3)oc3ccccc34)cc2)cc1 ; c1ccc(-c2ccc(N(c3ccc(-c4ccc5c(ccc6ccc7ccccc7c65)c4)cc3)c3cccc4c3oc3ccccc34)cc2)cc1 ; c1ccc(-c2ccc(N(c3ccc(-c4ccc5c(ccc6ccc7ccccc7c65)c4)cc3)c3ccc4oc5ccccc5c4c3)cc2)cc1]

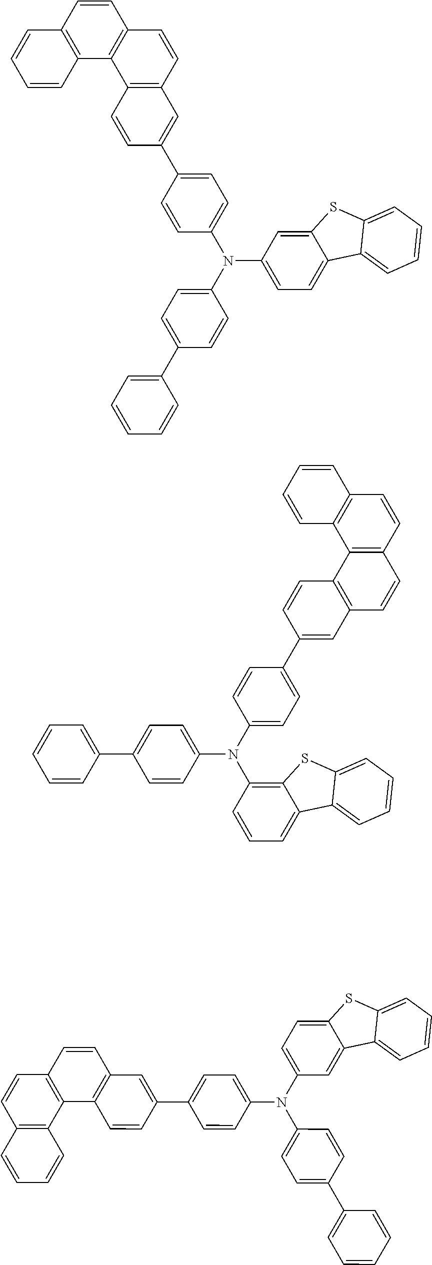 [SMILES: c1ccc(-c2ccc(N(c3ccc(-c4ccc5c(ccc6ccc7ccccc7c65)c4)cc3)c3ccc4c(c3)sc3ccccc34)cc2)cc1 ; c1ccc(-c2ccc(N(c3ccc(-c4ccc5c(ccc6ccc7ccccc7c65)c4)cc3)c3cccc4c3sc3ccccc34)cc2)cc1 ; c1ccc(-c2ccc(N(c3ccc(-c4ccc5c(ccc6ccc7ccccc7c65)c4)cc3)c3ccc4sc5ccccc5c4c3)cc2)cc1]

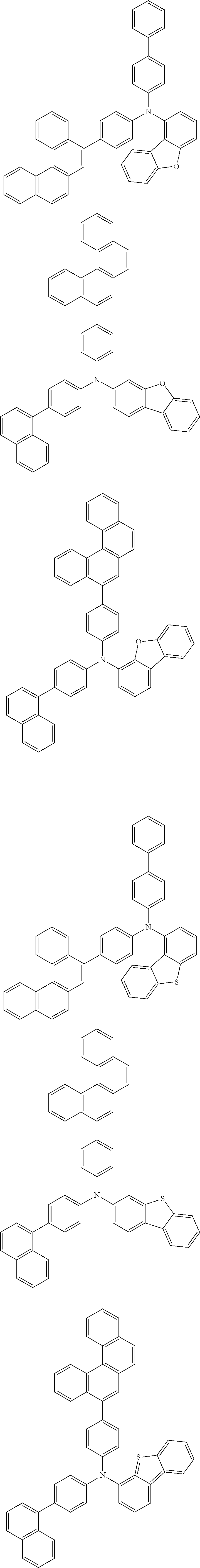

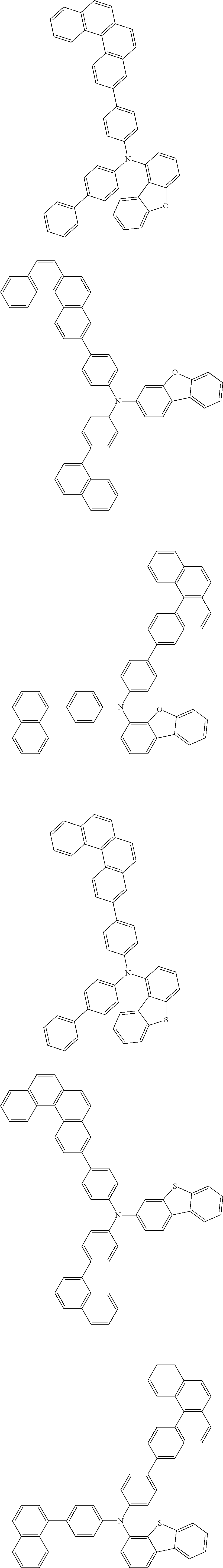

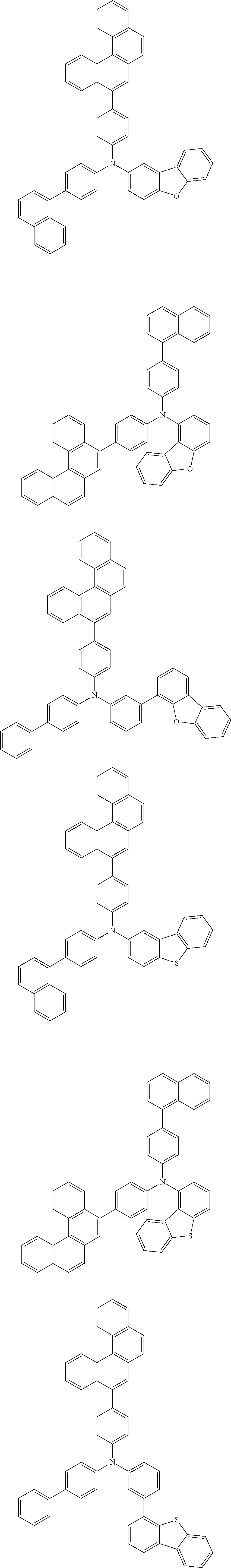

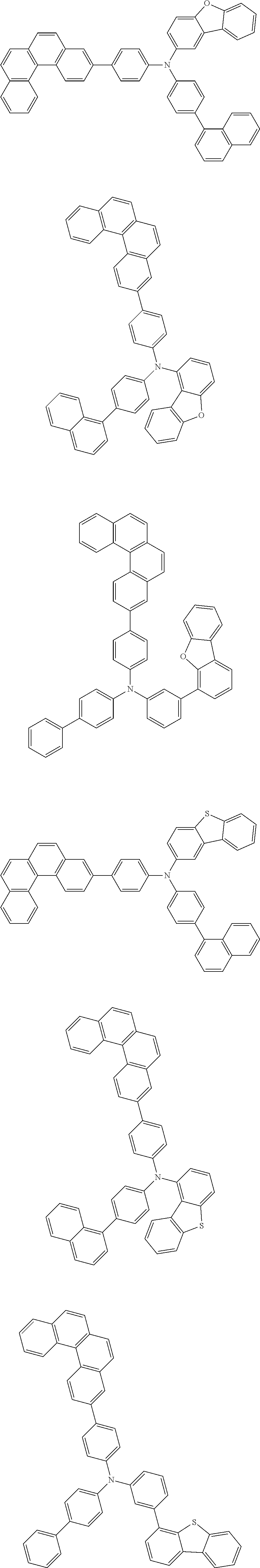

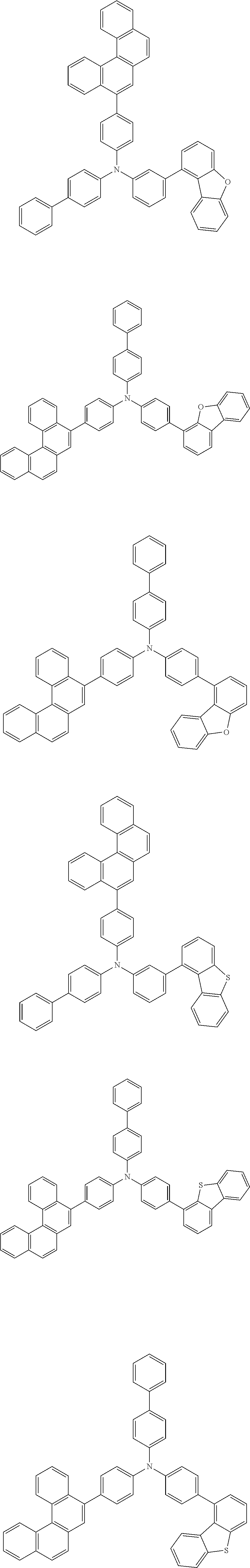

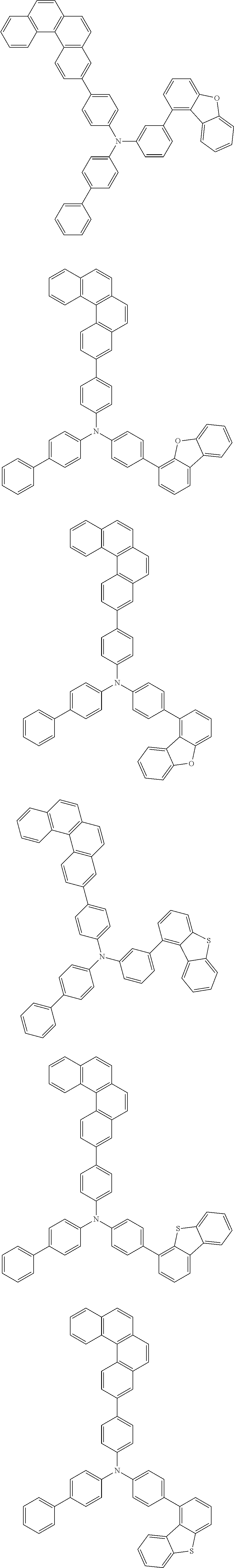

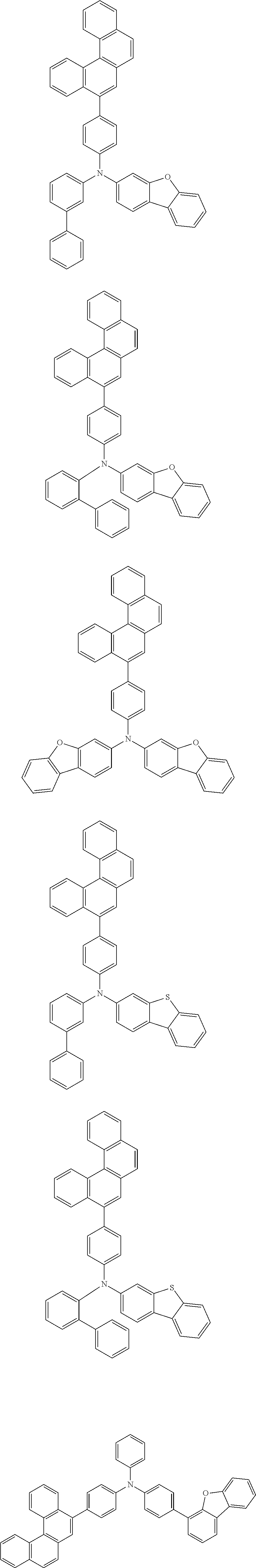

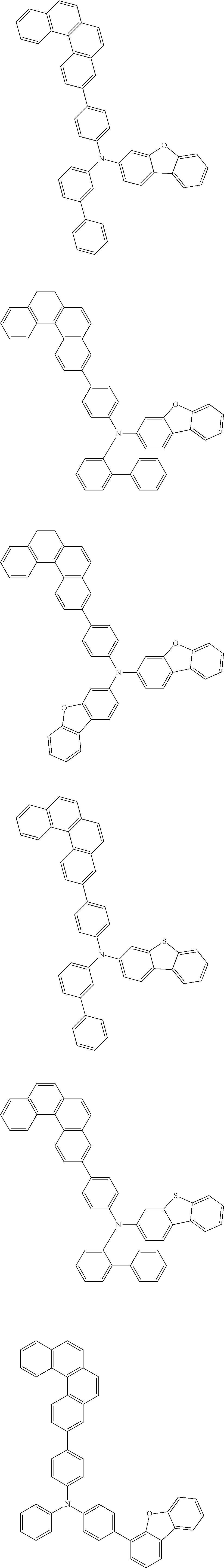

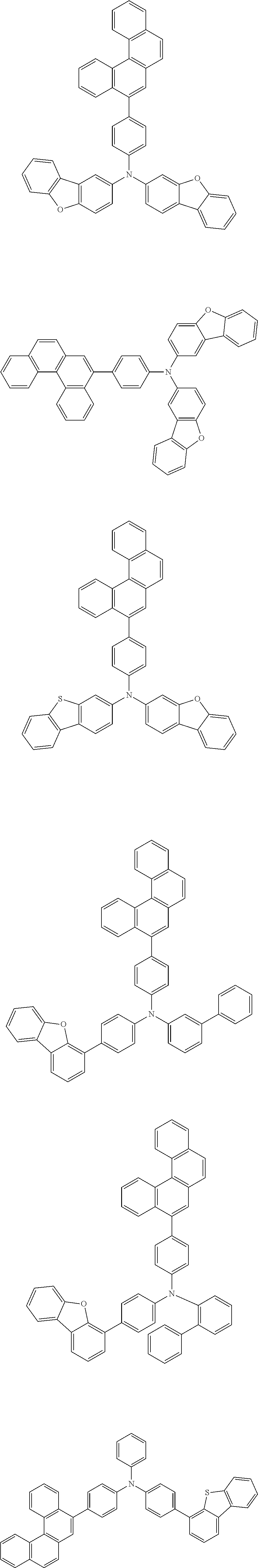

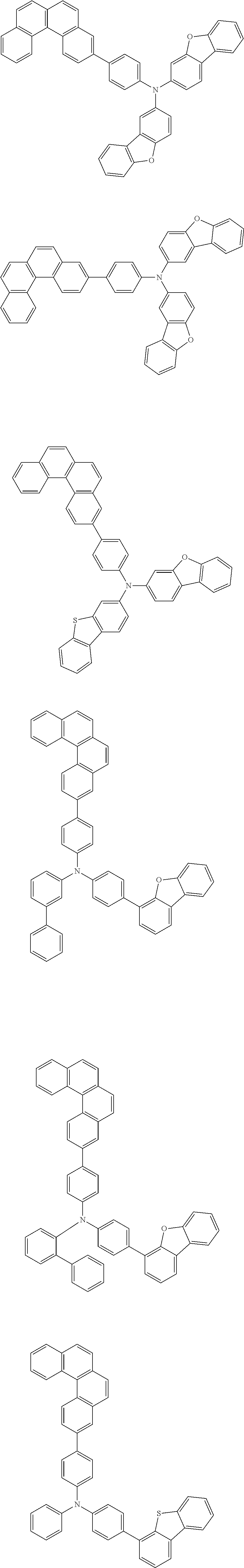

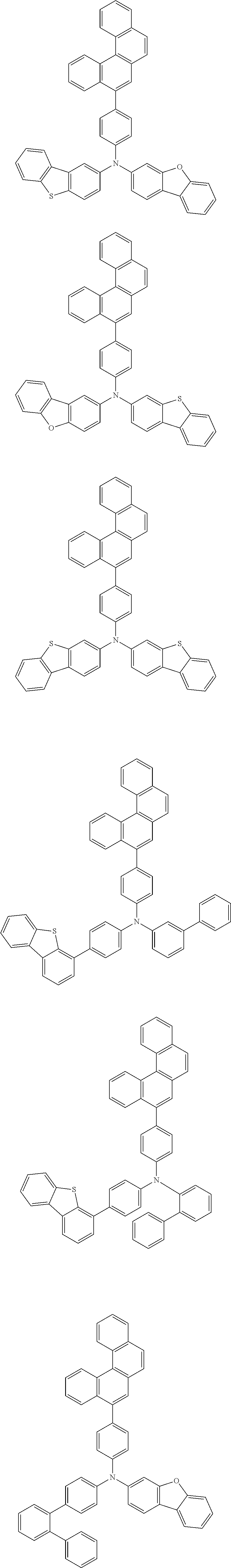

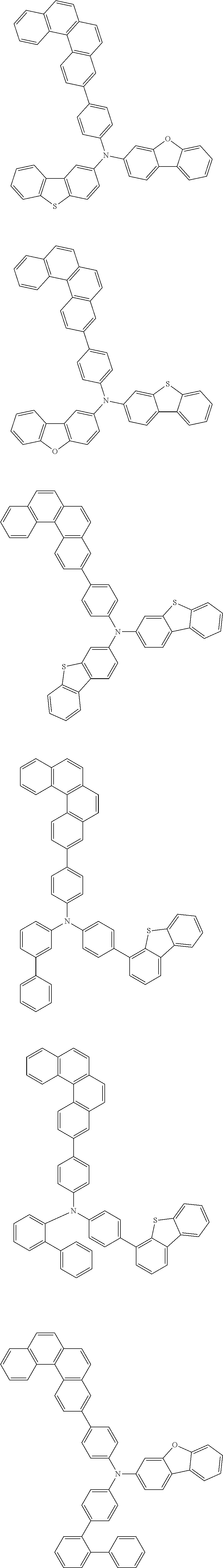

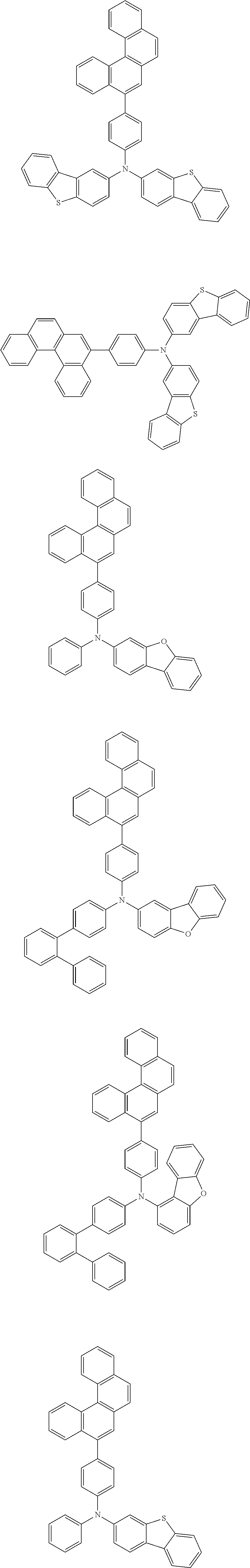

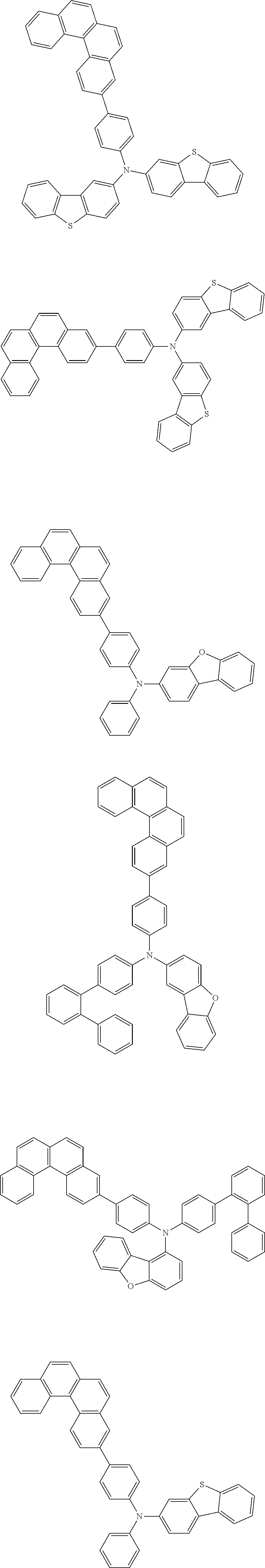

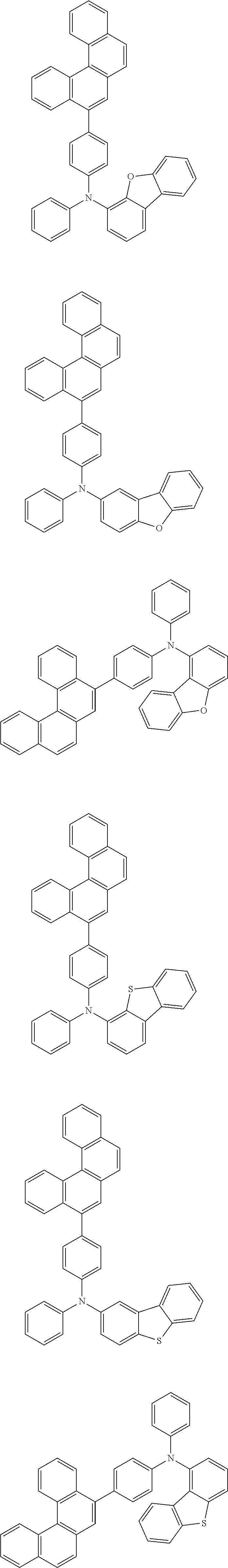 [SMILES: c1ccc(N(c2ccc(-c3cc4ccc5ccccc5c4c4ccccc34)cc2)c2cccc3c2oc2ccccc23)cc1 ; c1ccc(N(c2ccc(-c3cc4ccc5ccccc5c4c4ccccc34)cc2)c2ccc3oc4ccccc4c3c2)cc1 ; c1ccc(N(c2ccc(-c3cc4ccc5ccccc5c4c4ccccc34)cc2)c2cccc3oc4ccccc4c23)cc1 ; c1ccc(N(c2ccc(-c3cc4ccc5ccccc5c4c4ccccc34)cc2)c2cccc3c2sc2ccccc23)cc1 ; c1ccc(N(c2ccc(-c3cc4ccc5ccccc5c4c4ccccc34)cc2)c2ccc3sc4ccccc4c3c2)cc1 ; c1ccc(N(c2ccc(-c3cc4ccc5ccccc5c4c4ccccc34)cc2)c2cccc3sc4ccccc4c23)cc1]

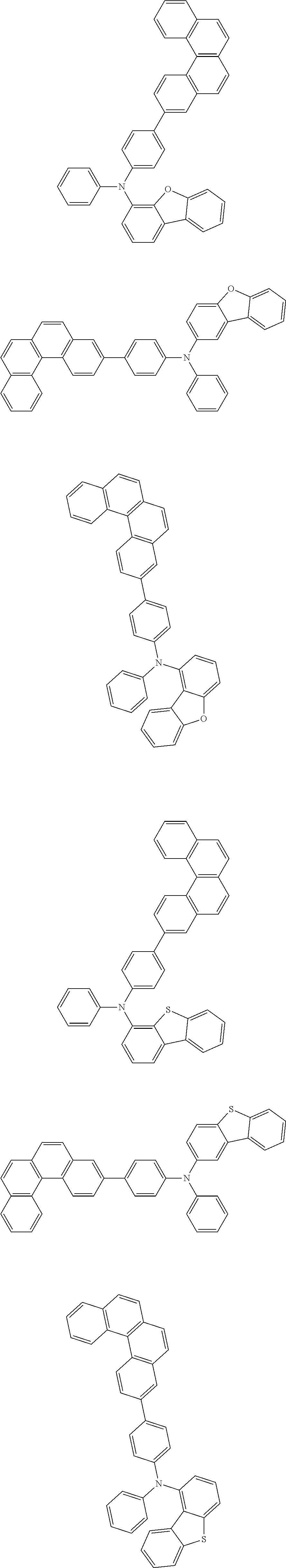 [SMILES: c1ccc(N(c2ccc(-c3ccc4c(ccc5ccc6ccccc6c54)c3)cc2)c2cccc3c2oc2ccccc23)cc1 ; c1ccc(N(c2ccc(-c3ccc4c(ccc5ccc6ccccc6c54)c3)cc2)c2ccc3oc4ccccc4c3c2)cc1 ; c1ccc(N(c2ccc(-c3ccc4c(ccc5ccc6ccccc6c54)c3)cc2)c2cccc3oc4ccccc4c23)cc1 ; c1ccc(N(c2ccc(-c3ccc4c(ccc5ccc6ccccc6c54)c3)cc2)c2cccc3c2sc2ccccc23)cc1 ; c1ccc(N(c2ccc(-c3ccc4c(ccc5ccc6ccccc6c54)c3)cc2)c2ccc3sc4ccccc4c3c2)cc1 ; c1ccc(N(c2ccc(-c3ccc4c(ccc5ccc6ccccc6c54)c3)cc2)c2cccc3sc4ccccc4c23)cc1]

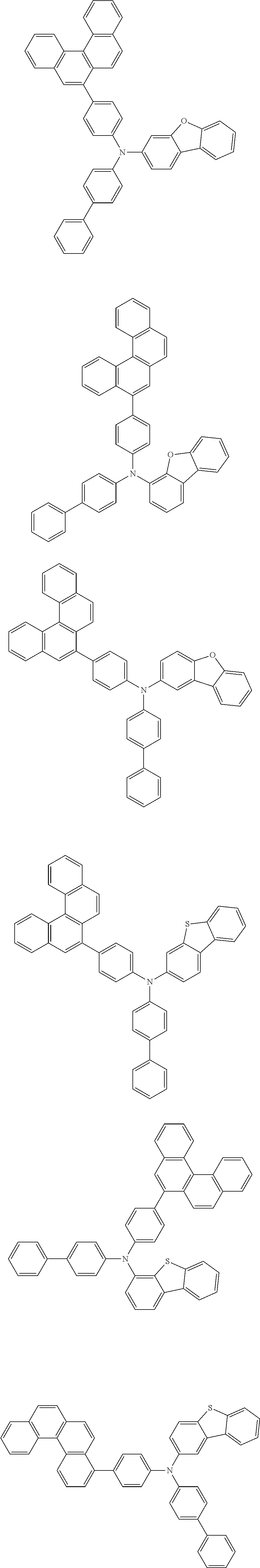

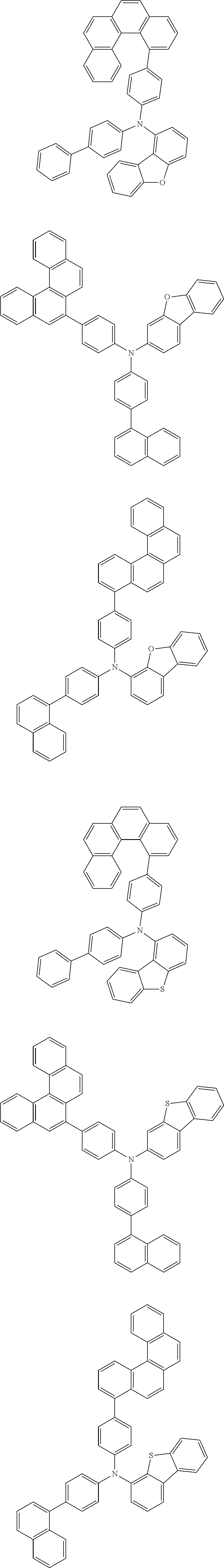

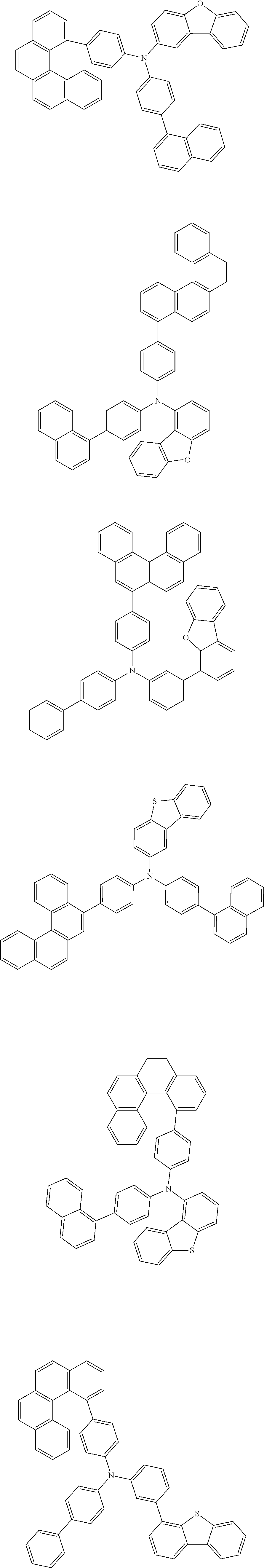

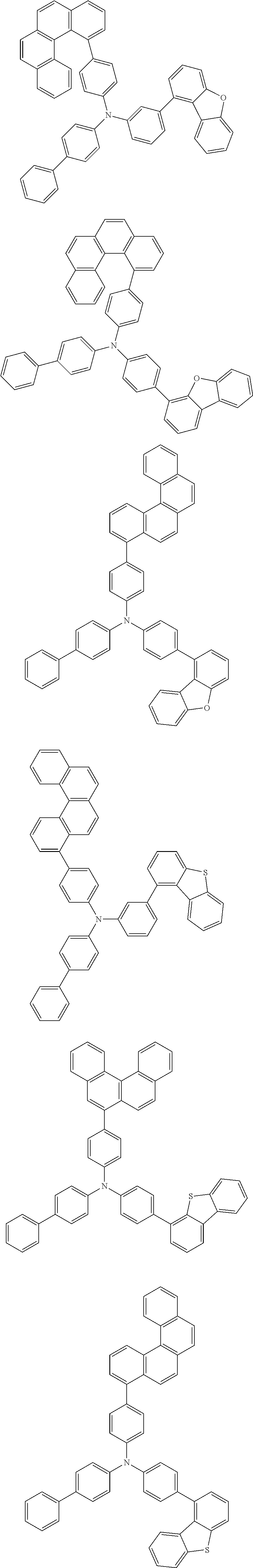

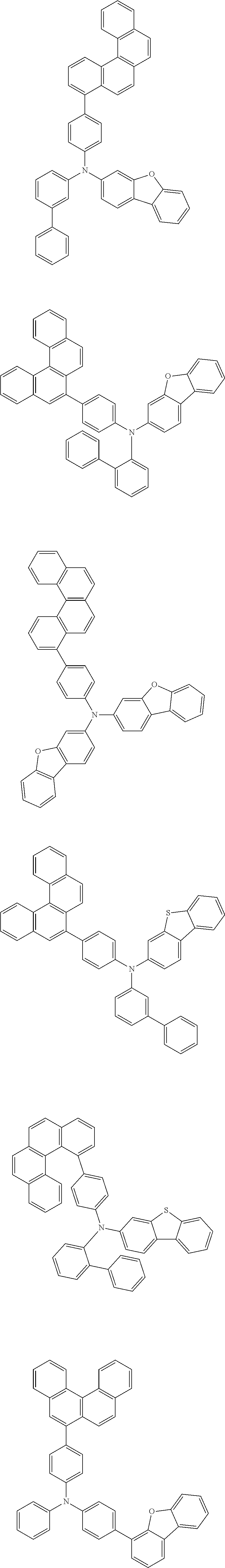

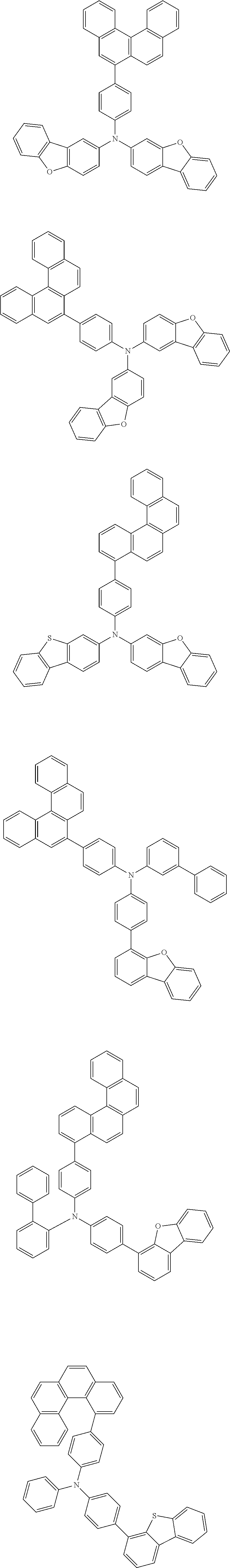

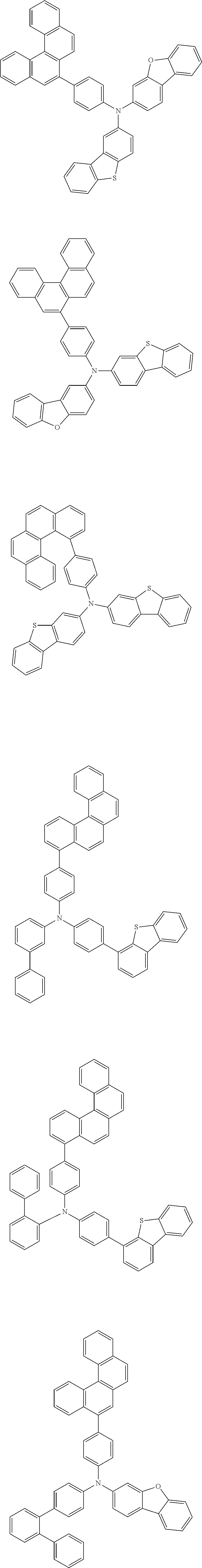

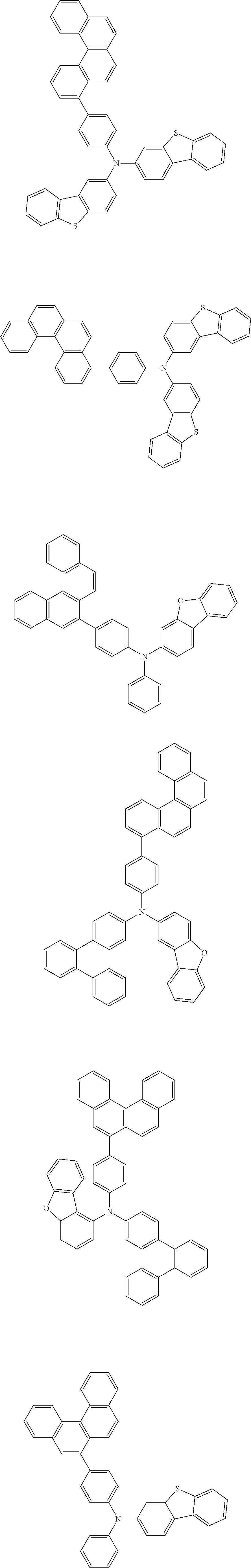

[chemical structure]

[chemical structure]

[chemical structure]

[chemical structure]

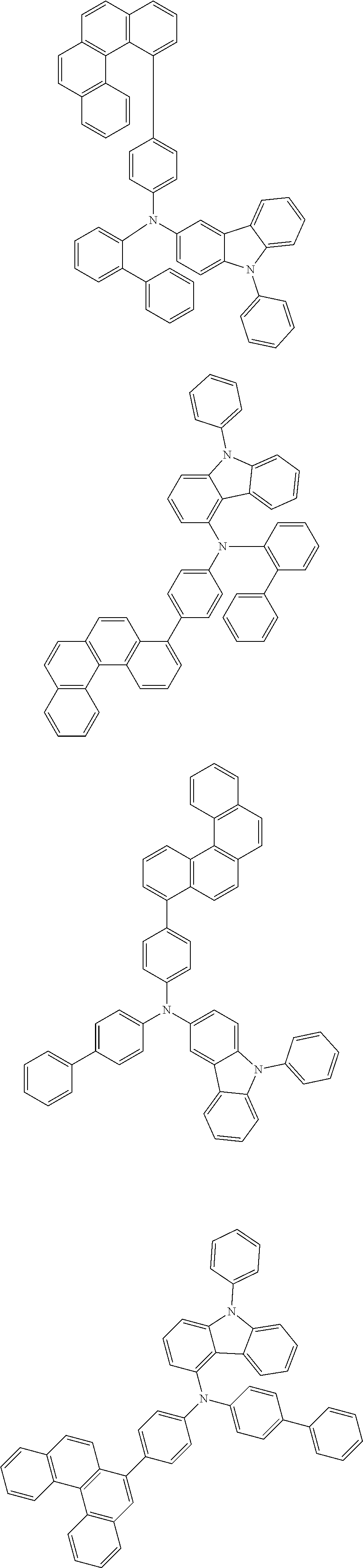 [SMILES: c1ccc(-c2ccccc2N(c2ccc(-c3cccc4ccc5ccc6ccccc6c5c34)cc2)c2ccc3c(c2)c2ccccc2n3-c2ccccc2)cc1 ; c1ccc(-c2ccccc2N(c2ccc(-c3cccc4c3ccc3ccc5ccccc5c34)cc2)c2cccc3c2c2ccccc2n3-c2ccccc2)cc1 ; c1ccc(-c2ccc(N(c3ccc(-c4cccc5c4ccc4ccc6ccccc6c45)cc3)c3ccc4c(c3)c3ccccc3n4-c3ccccc3)cc2)cc1 ; c1ccc(-c2ccc(N(c3ccc(-c4cc5ccccc5c5c4ccc4ccccc45)cc3)c3cccc4c3c3ccccc3n4-c3ccccc3)cc2)cc1]

[chemical structure]

[chemical structure]

[chemical structure]

[chemical structure]

[chemical structure]

[chemical structure]

[chemical structure]

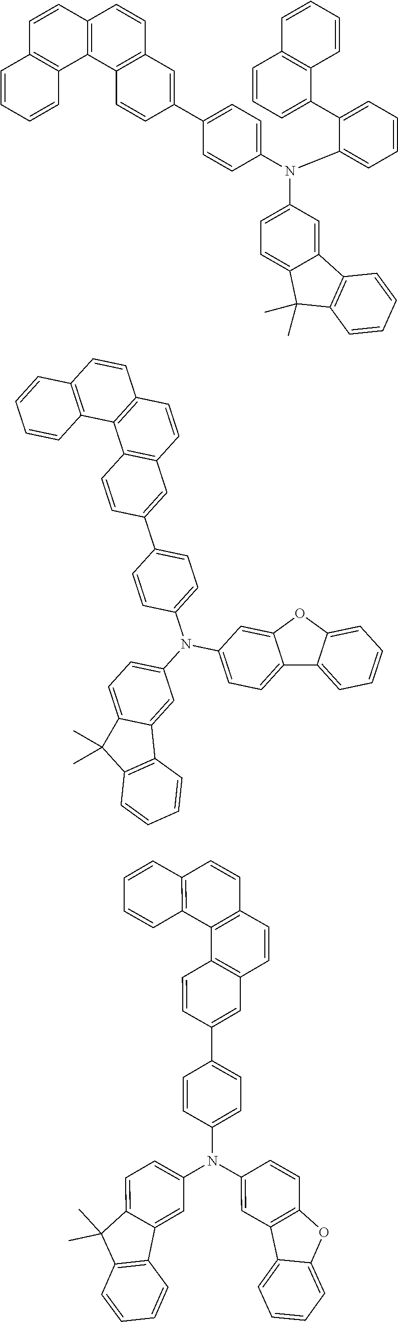

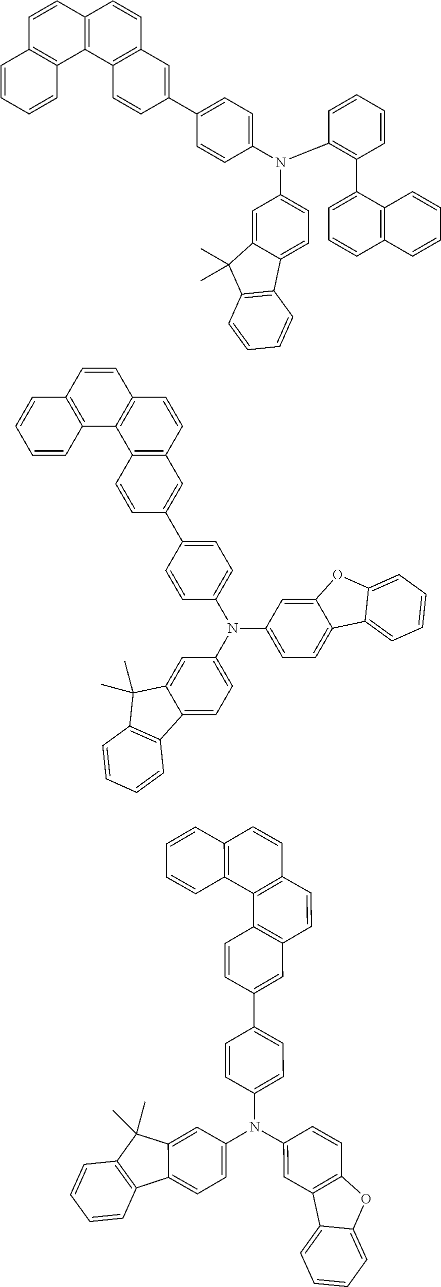 [SMILES: CC1(C)c2ccccc2-c2ccc(N(c3ccc(-c4ccc5c(ccc6ccc7ccccc7c65)c4)cc3)c3ccccc3-c3cccc4ccccc34)cc21 ; CC1(C)c2ccccc2-c2ccc(N(c3ccc(-c4ccc5c(ccc6ccc7ccccc7c65)c4)cc3)c3ccc4c(c3)oc3ccccc34)cc21 ; CC1(C)c2ccccc2-c2ccc(N(c3ccc(-c4ccc5c(ccc6ccc7ccccc7c65)c4)cc3)c3ccc4oc5ccccc5c4c3)cc21]

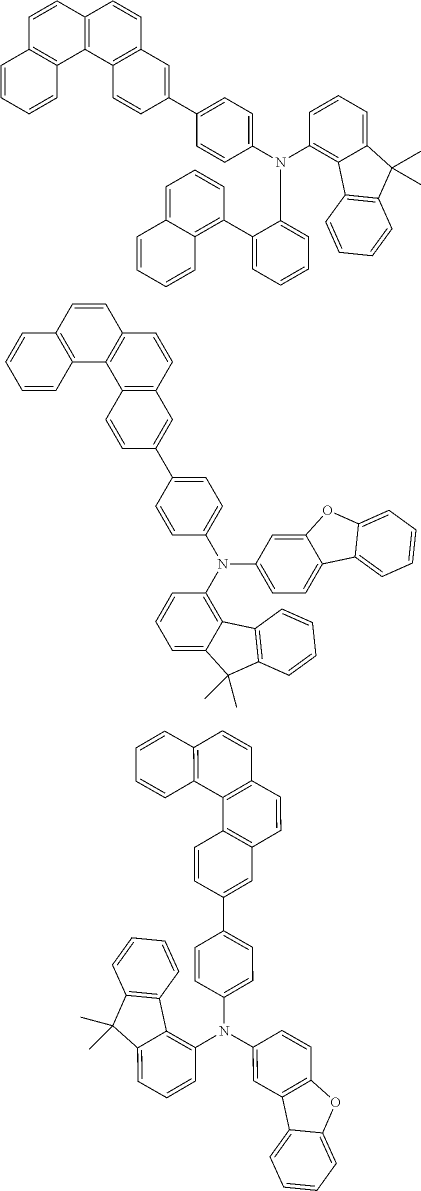

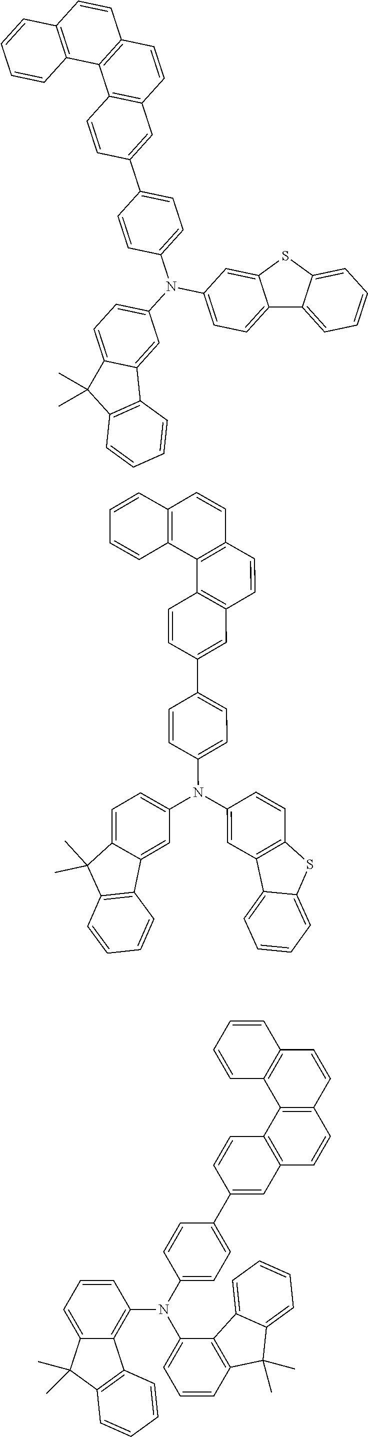

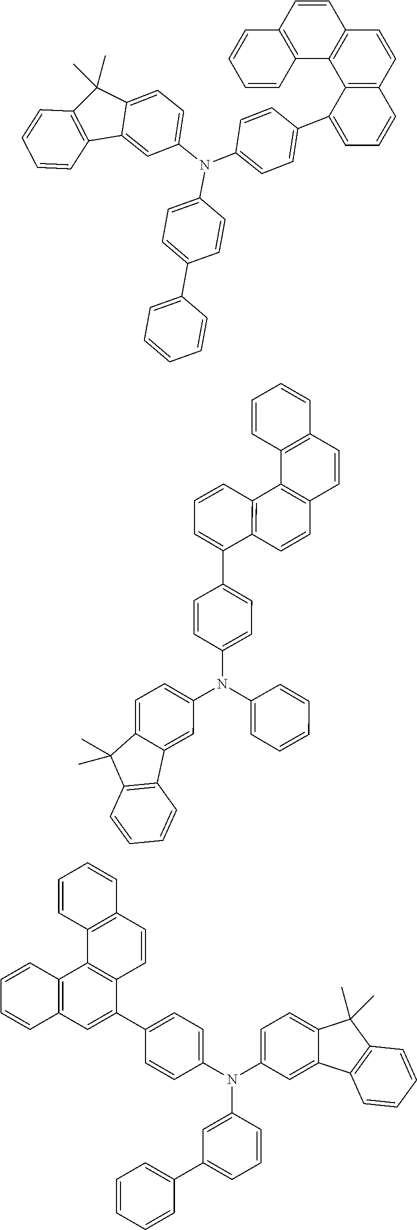

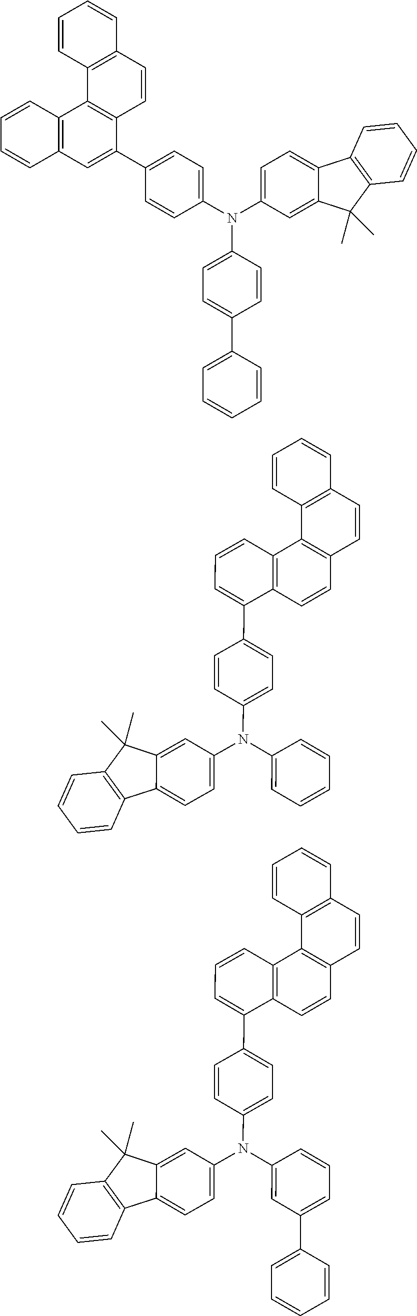

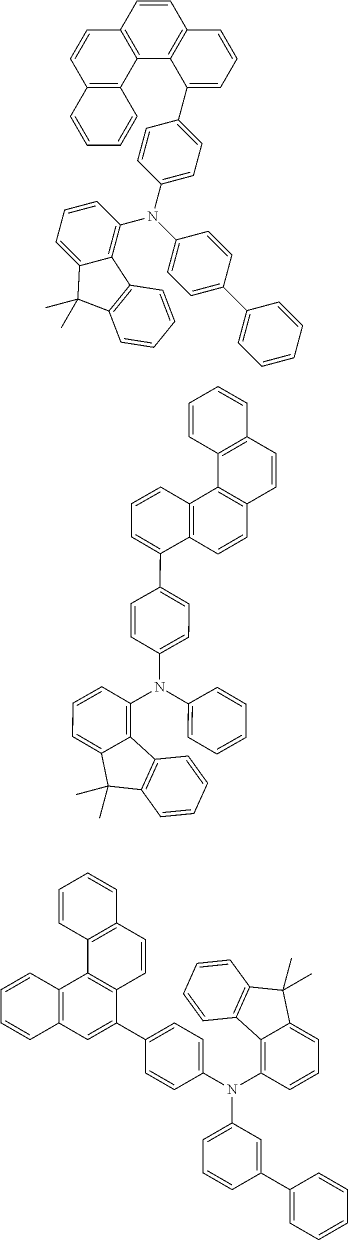

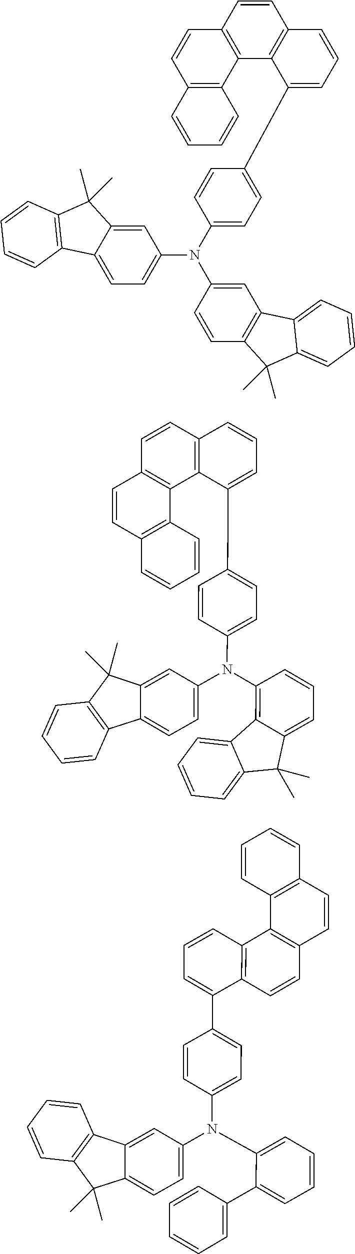

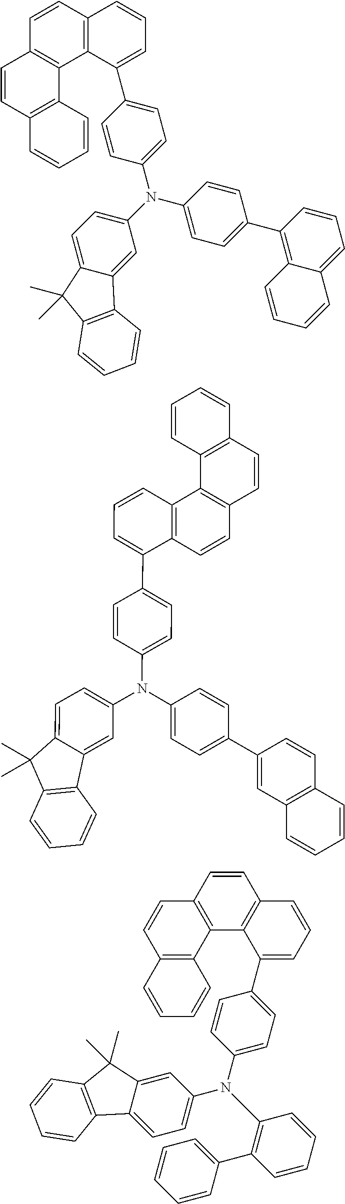 [SMILES: CC1(C)c2ccccc2-c2cc(N(c3ccc(-c4cccc5ccccc45)cc3)c3ccc(-c4cccc5ccc6ccc7ccccc7c6c45)cc3)ccc21 ; CC1(C)c2ccccc2-c2cc(N(c3ccc(-c4ccc5ccccc5c4)cc3)c3ccc(-c4cccc5c4ccc4ccc6ccccc6c45)cc3)ccc21 ; CC1(C)c2ccccc2-c2ccc(N(c3ccc(-c4cccc5ccc6ccc7ccccc7c6c45)cc3)c3ccccc3-c3ccccc3)cc21]

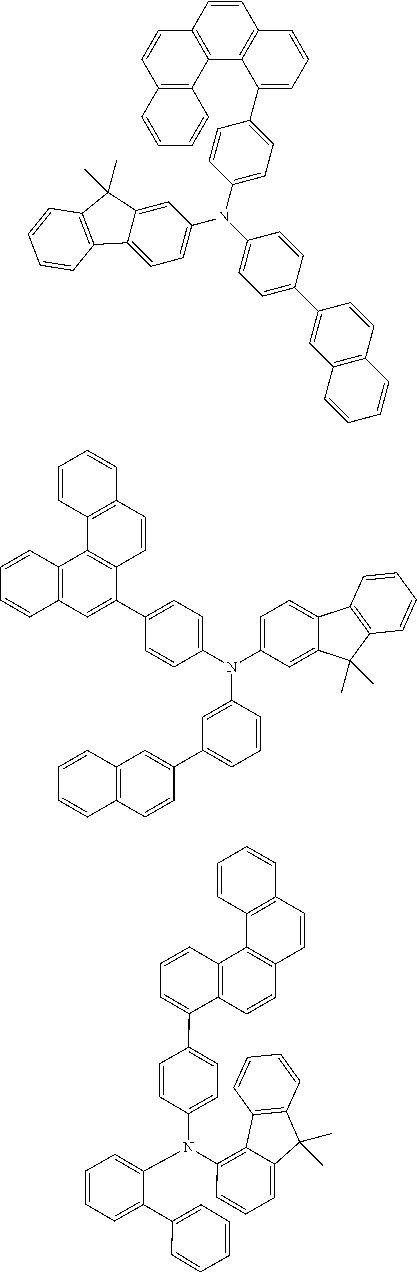 [SMILES: CC1(C)c2ccccc2-c2ccc(N(c3ccc(-c4ccc5ccccc5c4)cc3)c3ccc(-c4cccc5ccc6ccc7ccccc7c6c45)cc3)cc21 ; CC1(C)c2ccccc2-c2ccc(N(c3ccc(-c4cc5ccccc5c5c4ccc4ccccc45)cc3)c3cccc(-c4ccc5ccccc5c4)c3)cc21 ; CC1(C)c2ccccc2-c2c(N(c3ccc(-c4cccc5c4ccc4ccc6ccccc6c45)cc3)c3ccccc3-c3ccccc3)cccc21]

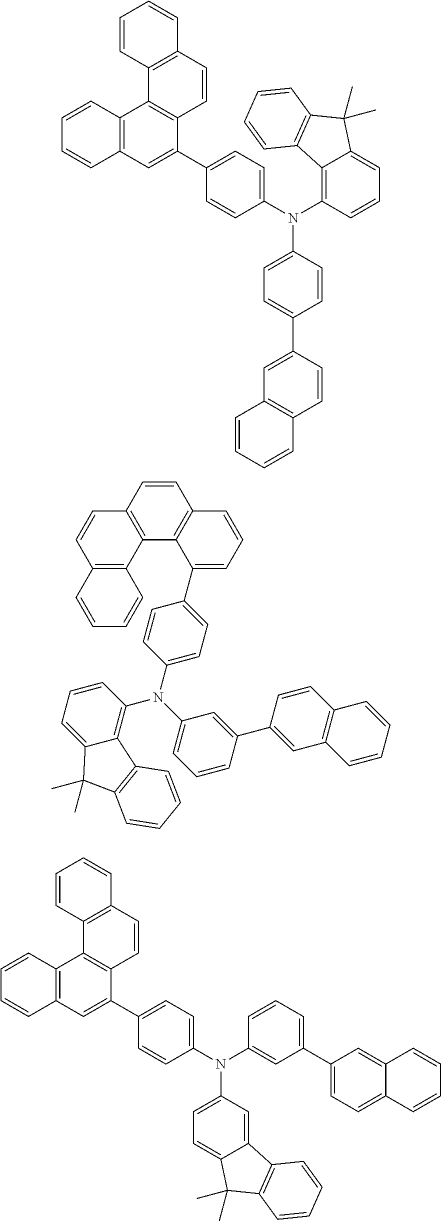

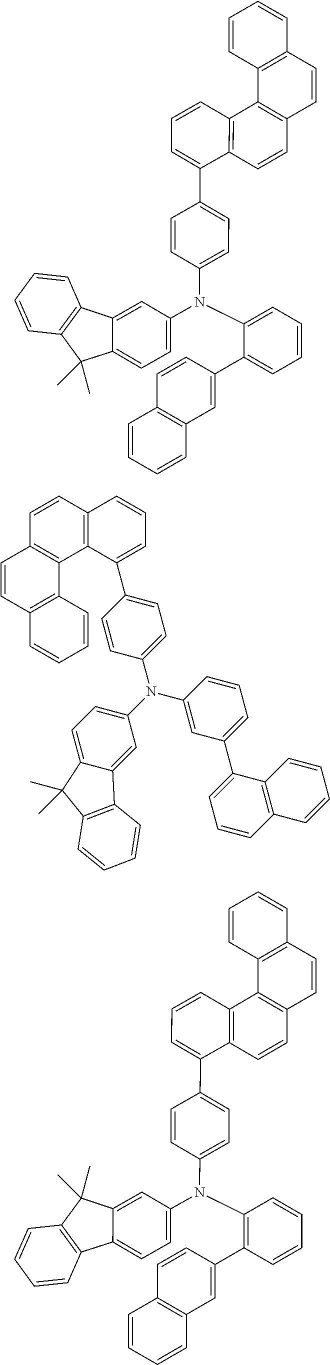

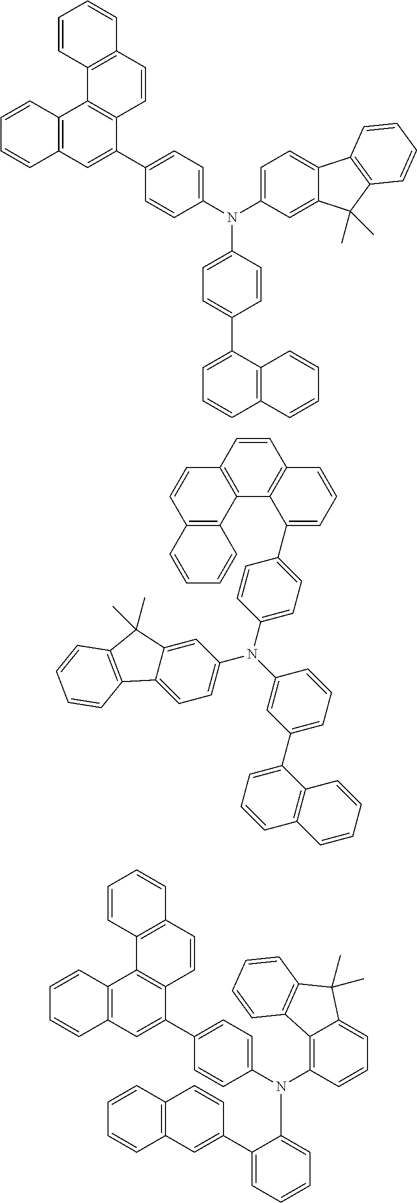

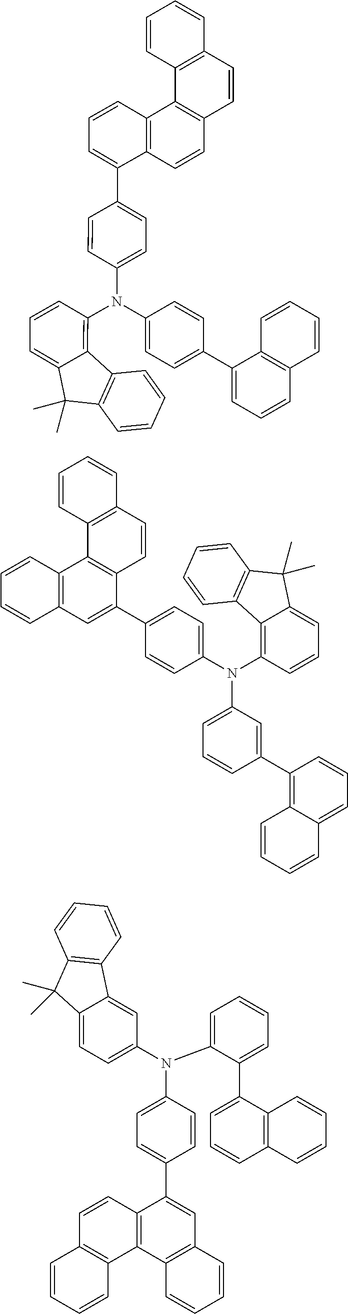

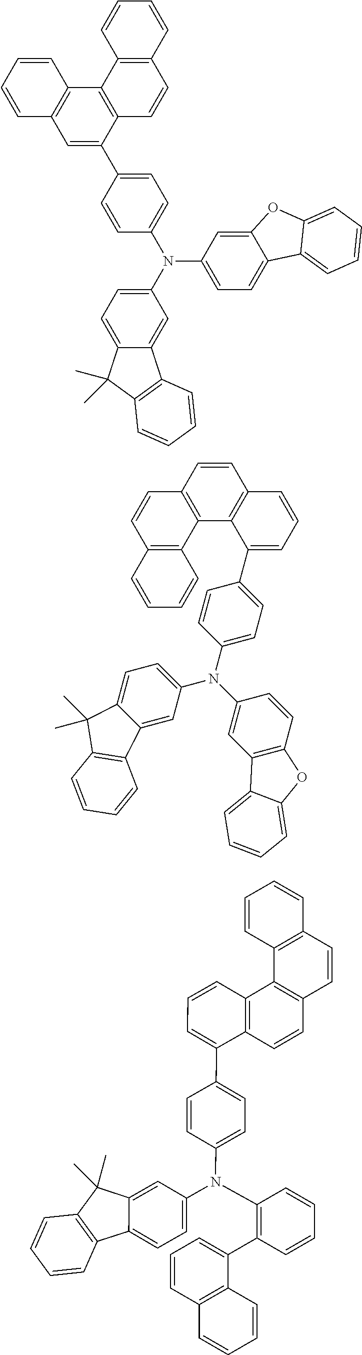

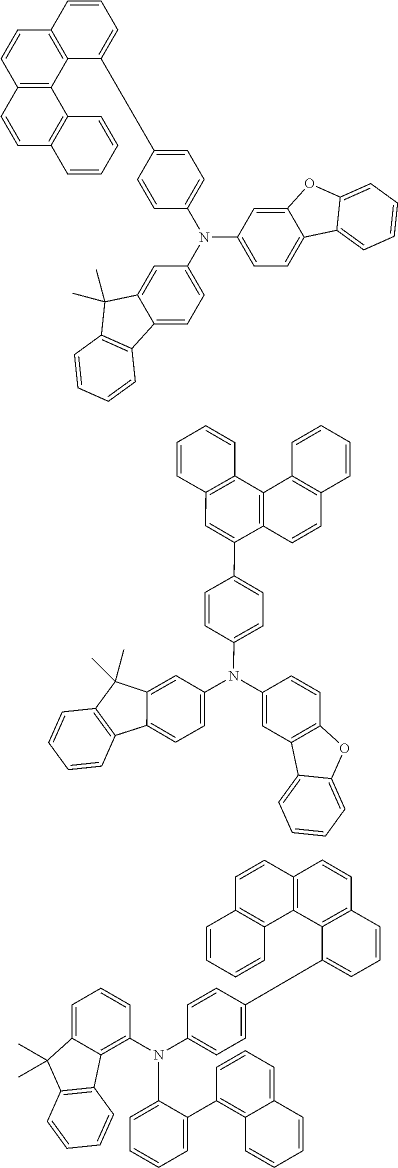

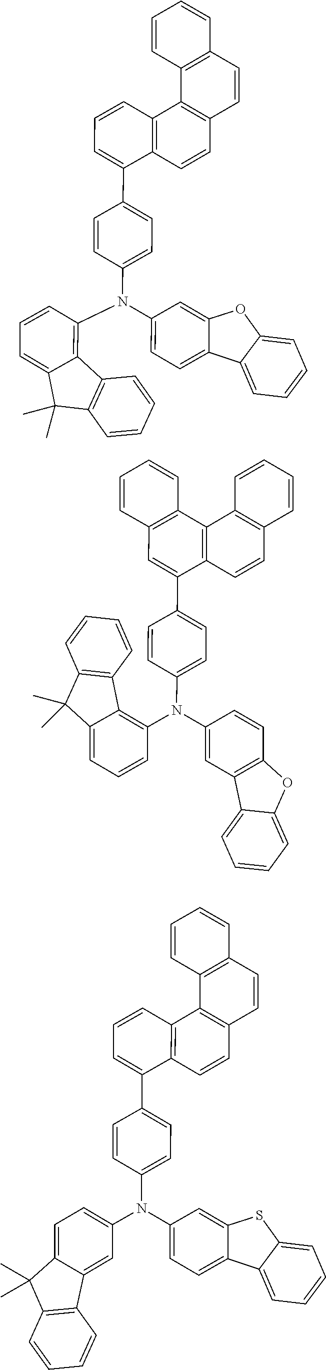

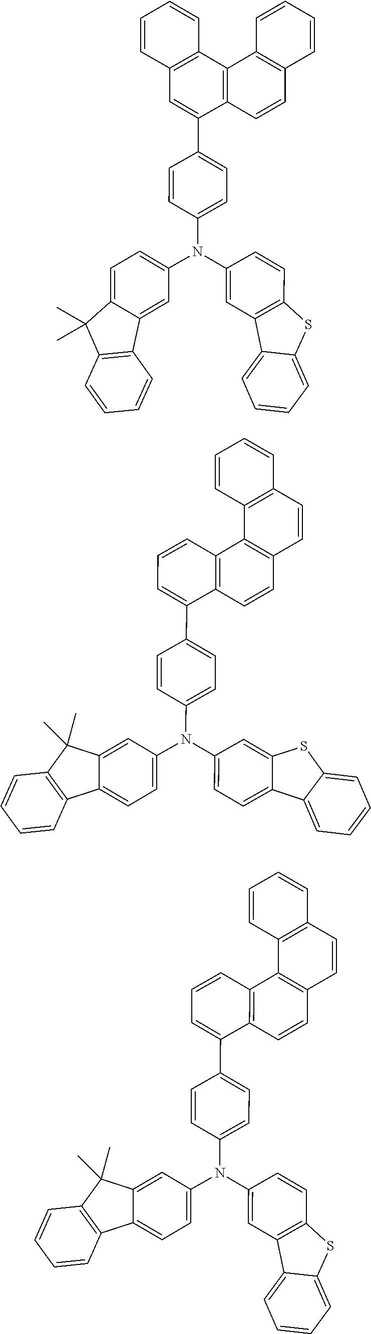

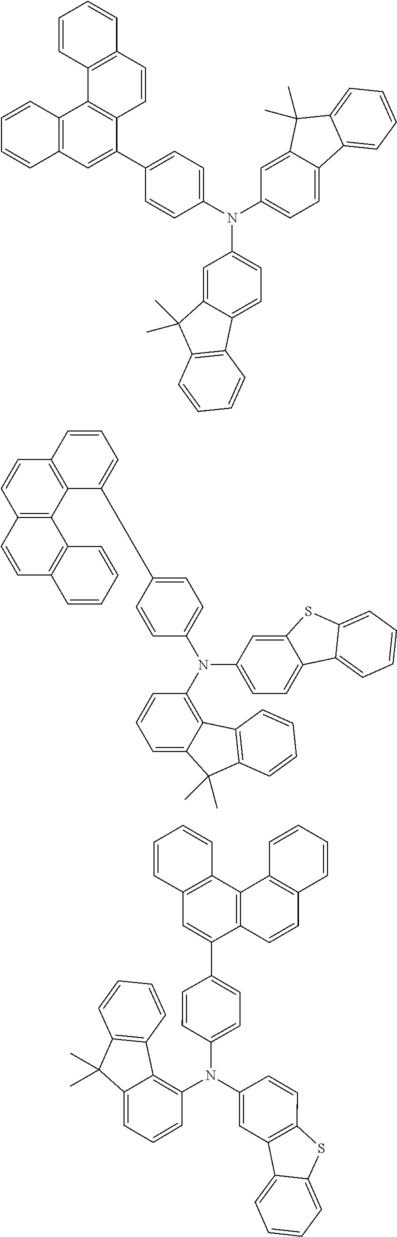

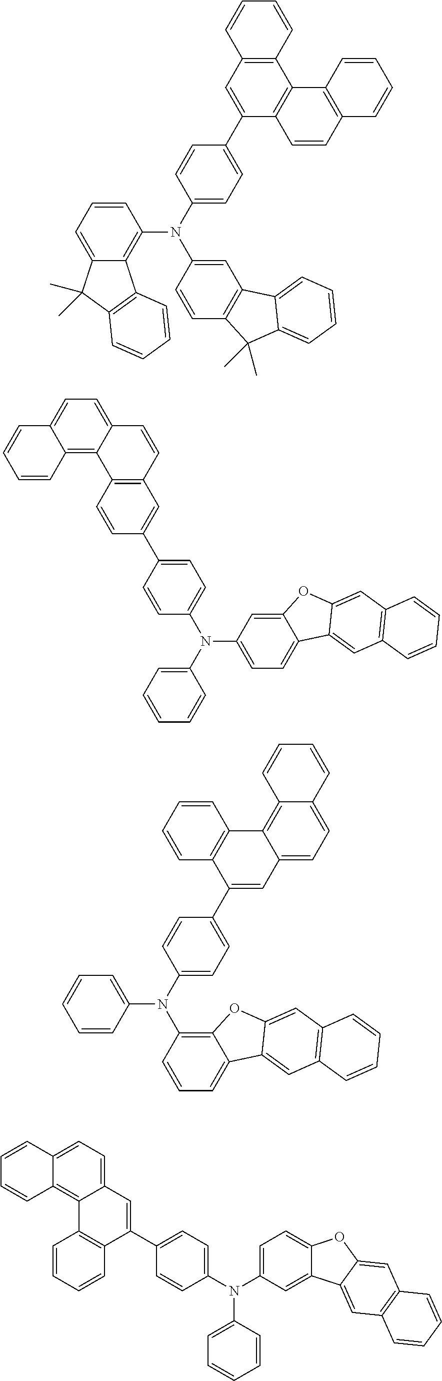 [SMILES: CC1(C)c2ccccc2-c2cc(N(c3ccc(-c4cc5ccccc5c5c4ccc4ccccc45)cc3)c3cccc4c3-c3ccccc3C4(C)C)ccc21 ; c1ccc(N(c2ccc(-c3ccc4c(ccc5ccc6ccccc6c54)c3)cc2)c2ccc3c(c2)oc2cc4ccccc4cc23)cc1 ; c1ccc(N(c2ccc(-c3cc4ccc5ccccc5c4c4ccccc34)cc2)c2cccc3c2oc2cc4ccccc4cc23)cc1 ; c1ccc(N(c2ccc(-c3cc4ccc5ccccc5c4c4ccccc34)cc2)c2ccc3oc4cc5ccccc5cc4c3c2)cc1]

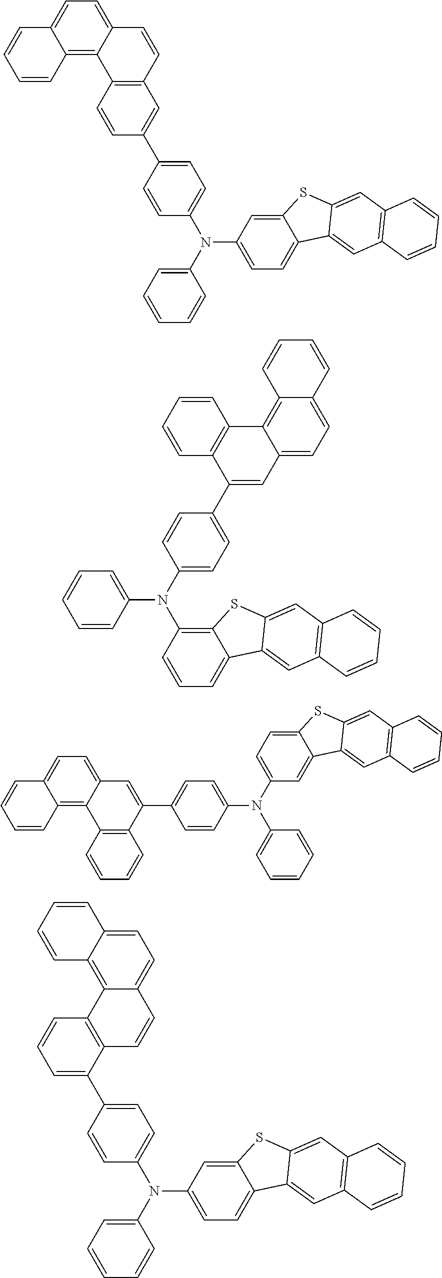

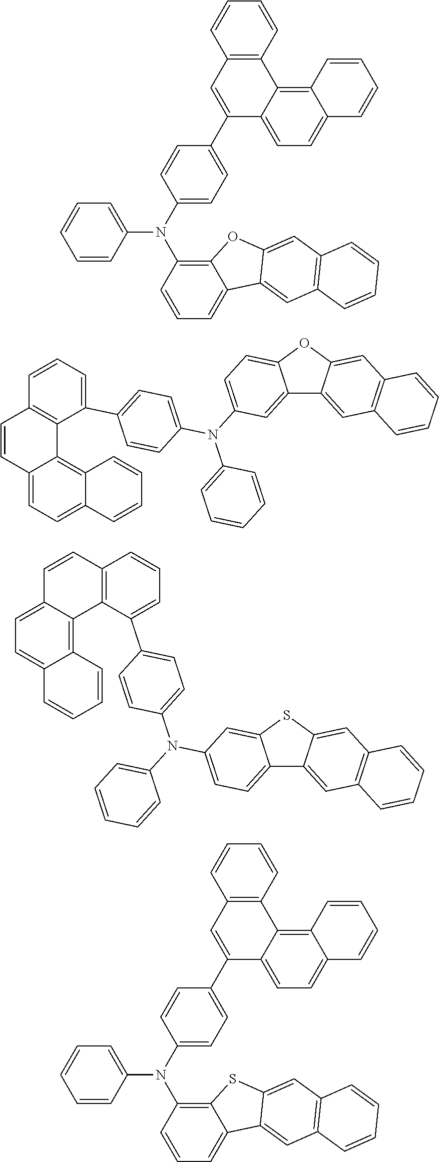

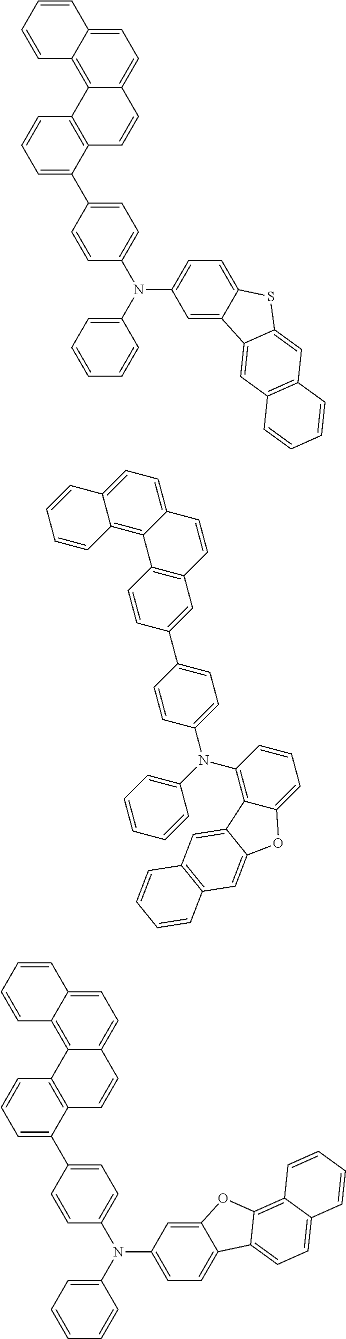 [SMILES: c1ccc(N(c2ccc(-c3cccc4c3ccc3ccc5ccccc5c34)cc2)c2ccc3sc4cc5ccccc5cc4c3c2)cc1 ; c1ccc(N(c2ccc(-c3ccc4c(ccc5ccc6ccccc6c54)c3)cc2)c2cccc3oc4cc5ccccc5cc4c23)cc1 ; c1ccc(N(c2ccc(-c3cccc4c3ccc3ccc5ccccc5c34)cc2)c2ccc3c(c2)oc2c4ccccc4ccc32)cc1]

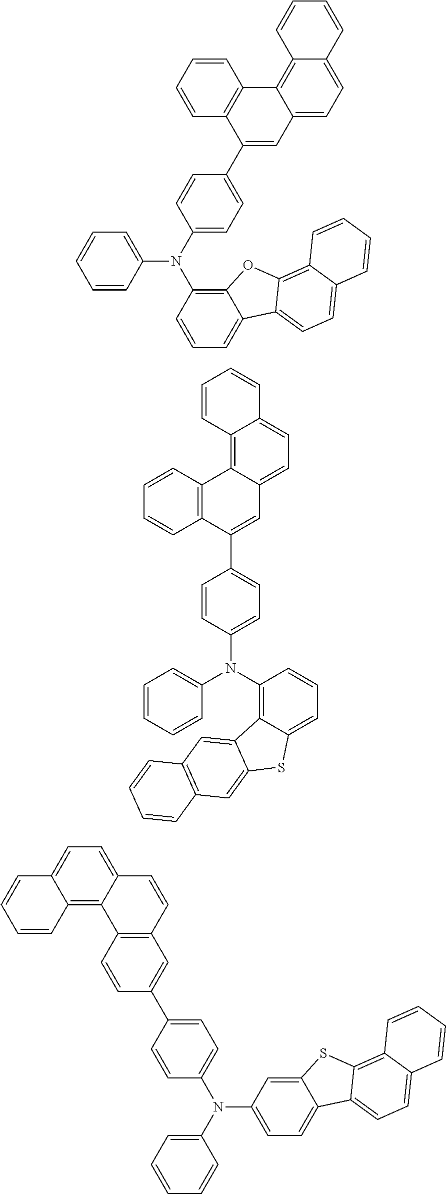

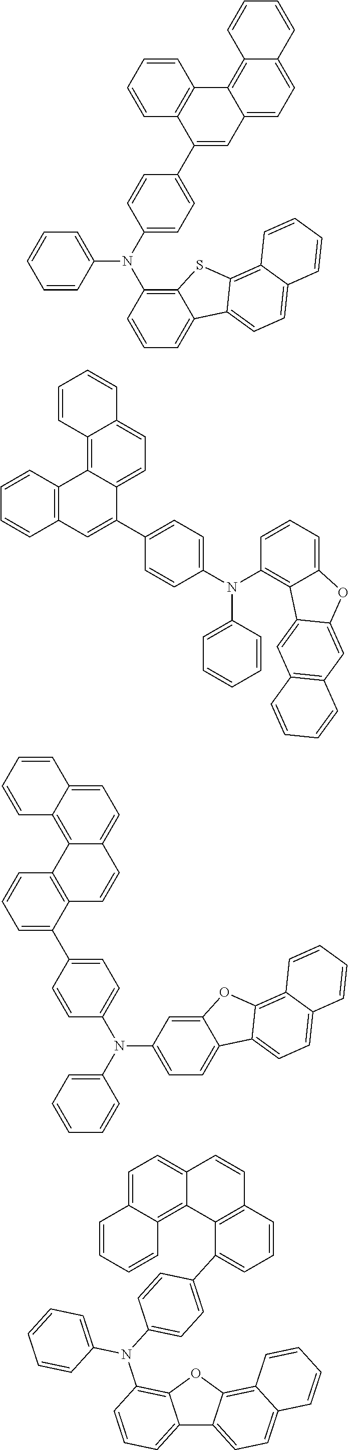 [SMILES: c1ccc(N(c2ccc(-c3cc4ccc5ccccc5c4c4ccccc34)cc2)c2cccc3c2sc2c4ccccc4ccc32)cc1 ; c1ccc(N(c2ccc(-c3cc4ccccc4c4c3ccc3ccccc34)cc2)c2cccc3oc4cc5ccccc5cc4c23)cc1 ; c1ccc(N(c2ccc(-c3cccc4c3ccc3ccc5ccccc5c34)cc2)c2ccc3c(c2)oc2c4ccccc4ccc32)cc1 ; c1ccc(N(c2ccc(-c3cccc4ccc5ccc6ccccc6c5c34)cc2)c2cccc3c2oc2c4ccccc4ccc32)cc1]

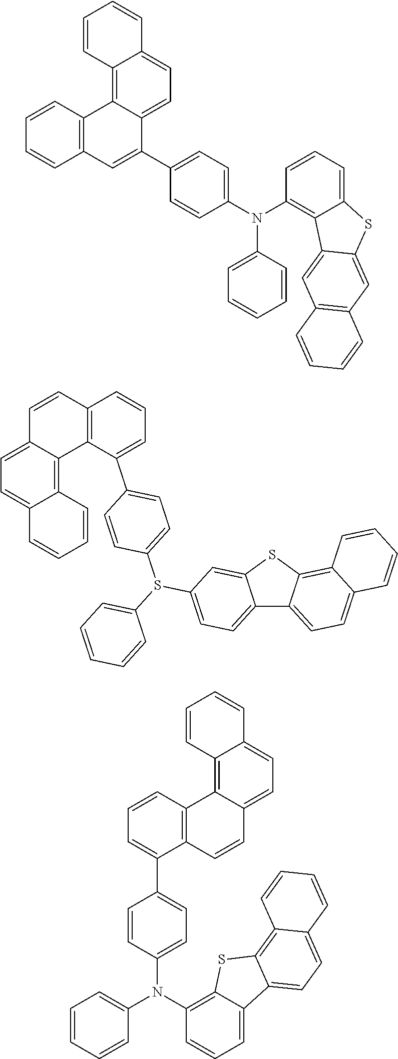

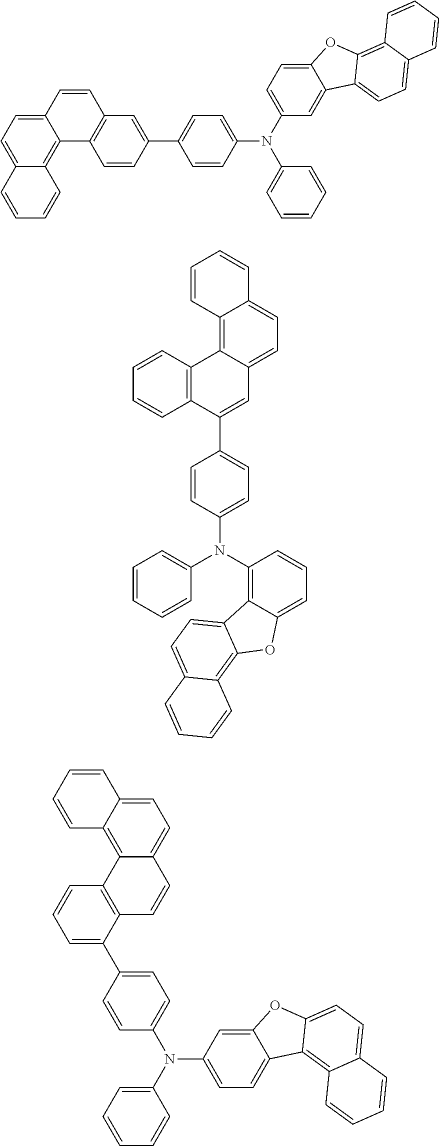 [SMILES: c1ccc(N(c2ccc(-c3ccc4c(ccc5ccc6ccccc6c54)c3)cc2)c2ccc3oc4c5ccccc5ccc4c3c2)cc1 ; c1ccc(N(c2ccc(-c3cc4ccc5ccccc5c4c4ccccc34)cc2)c2cccc3oc4c5ccccc5ccc4c23)cc1 ; c1ccc(N(c2ccc(-c3cccc4c3ccc3ccc5ccccc5c34)cc2)c2ccc3c(c2)oc2ccc4ccccc4c23)cc1]

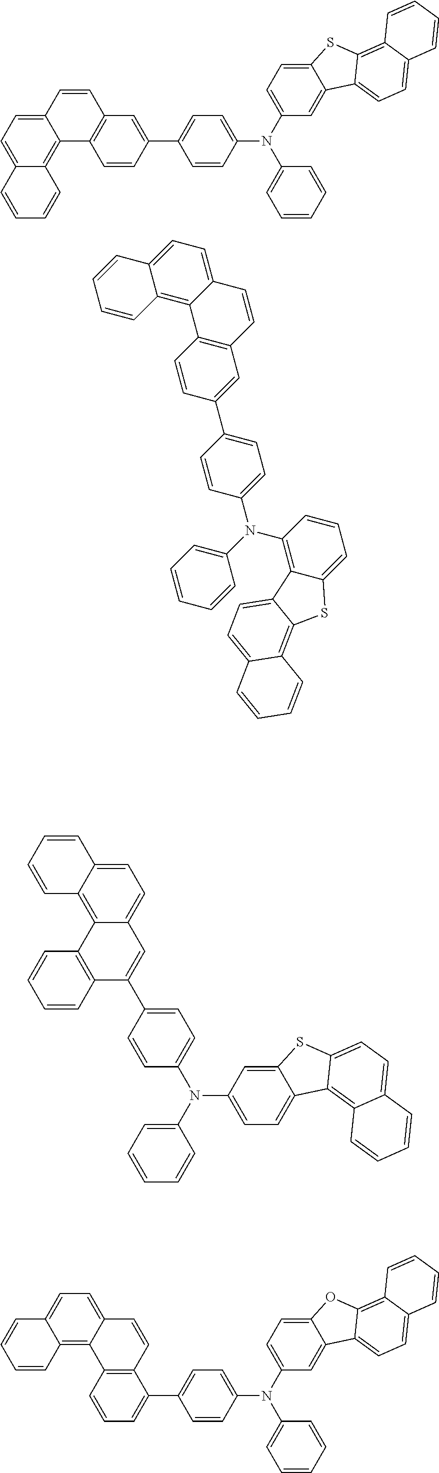 [SMILES: c1ccc(N(c2ccc(-c3ccc4c(ccc5ccc6ccccc6c54)c3)cc2)c2ccc3sc4c5ccccc5ccc4c3c2)cc1 ; c1ccc(N(c2ccc(-c3ccc4c(ccc5ccc6ccccc6c54)c3)cc2)c2cccc3sc4c5ccccc5ccc4c23)cc1 ; c1ccc(N(c2ccc(-c3cc4ccc5ccccc5c4c4ccccc34)cc2)c2ccc3c(c2)sc2ccc4ccccc4c23)cc1 ; c1ccc(N(c2ccc(-c3cccc4c3ccc3ccc5ccccc5c34)cc2)c2ccc3oc4c5ccccc5ccc4c3c2)cc1]

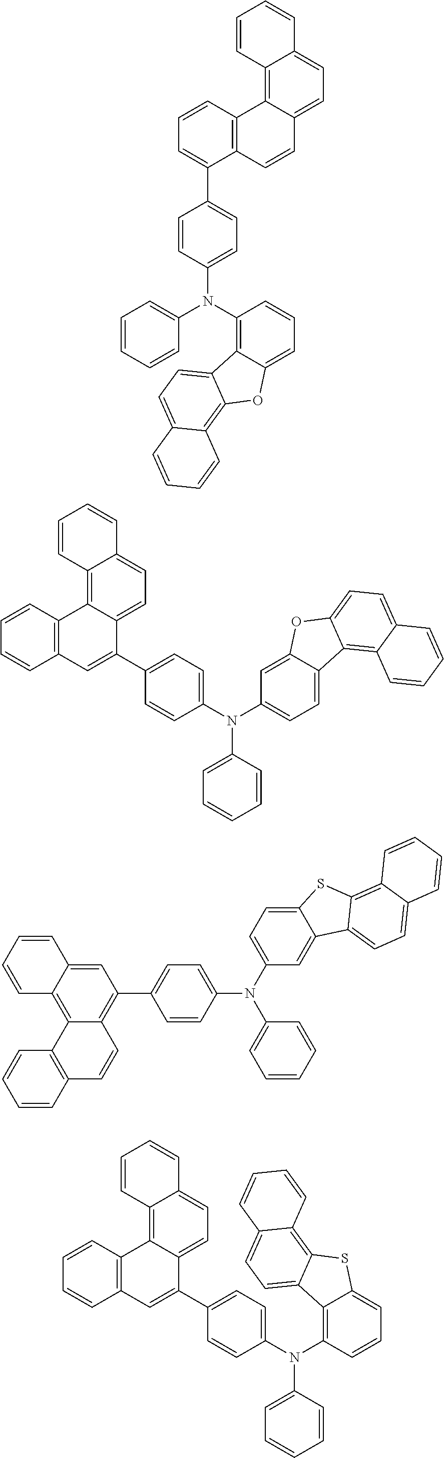 [SMILES: c1ccc(N(c2ccc(-c3cccc4c3ccc3ccc5ccccc5c34)cc2)c2cccc3oc4c5ccccc5ccc4c23)cc1 ; c1ccc(N(c2ccc(-c3cc4ccccc4c4c3ccc3ccccc34)cc2)c2ccc3c(c2)oc2ccc4ccccc4c23)cc1 ; c1ccc(N(c2ccc(-c3cc4ccccc4c4c3ccc3ccccc34)cc2)c2ccc3sc4c5ccccc5ccc4c3c2)cc1 ; c1ccc(N(c2ccc(-c3cc4ccccc4c4c3ccc3ccccc34)cc2)c2cccc3sc4c5ccccc5ccc4c23)cc1]

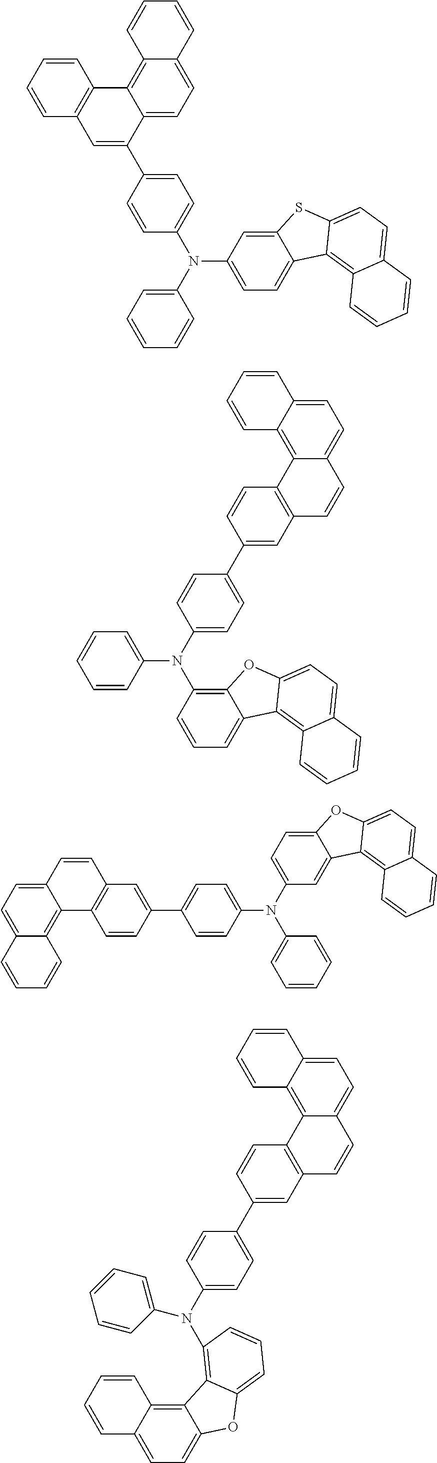

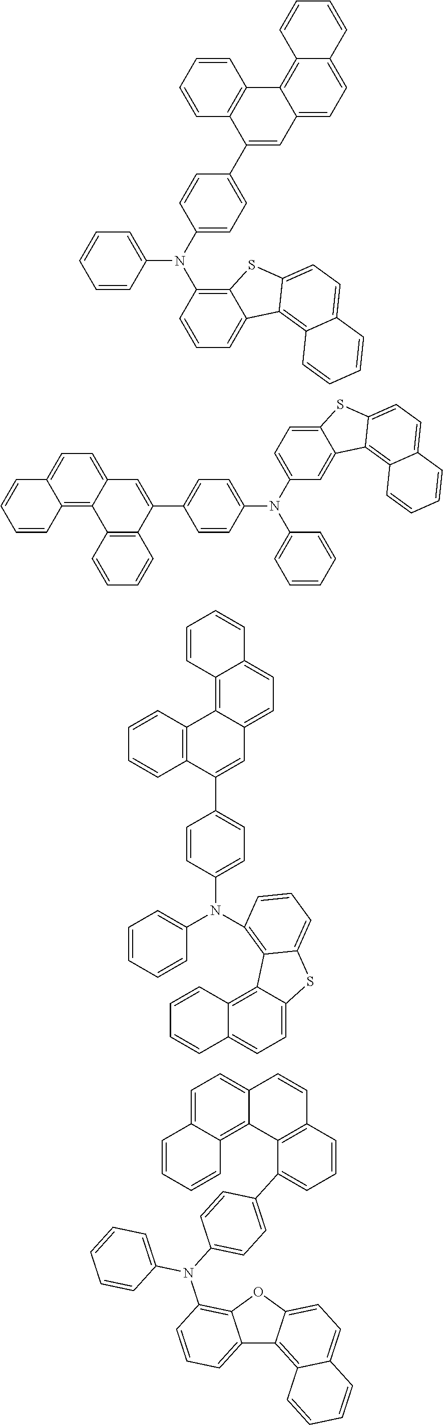

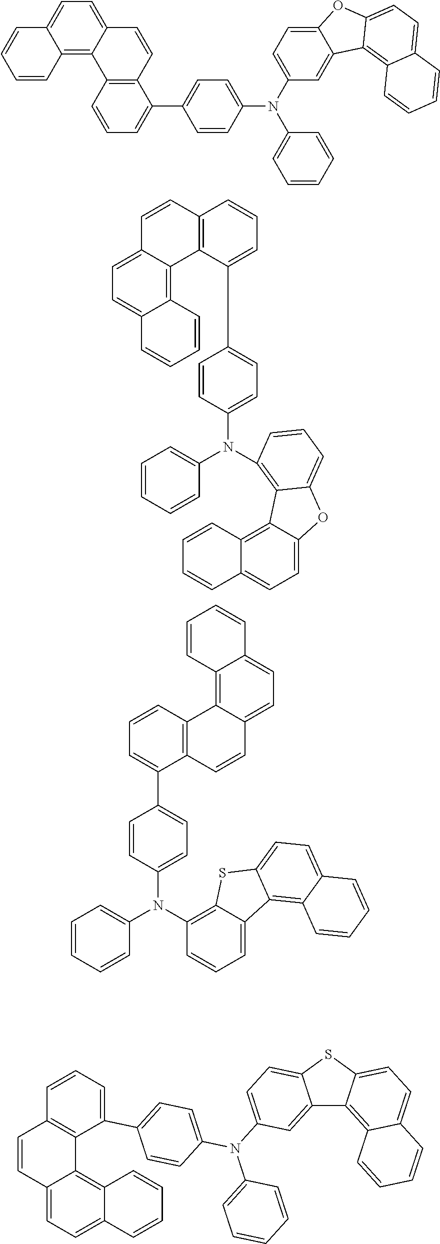

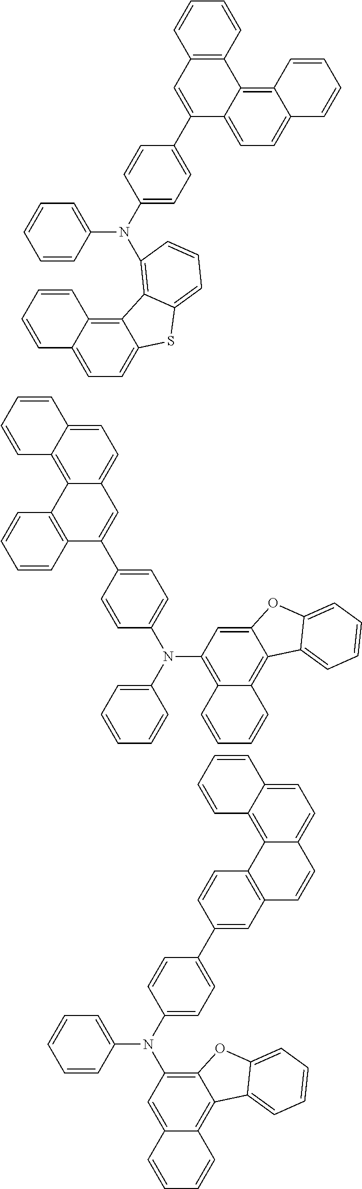

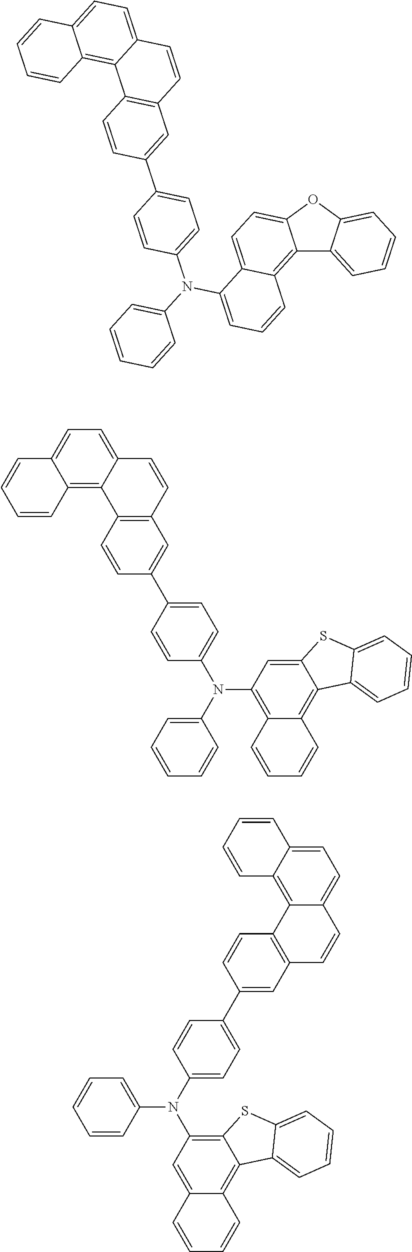

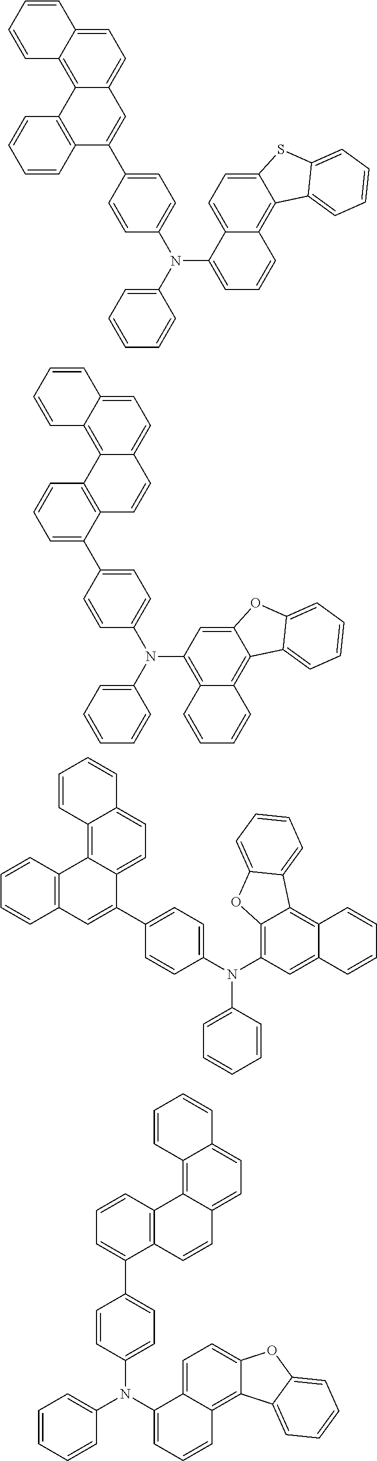

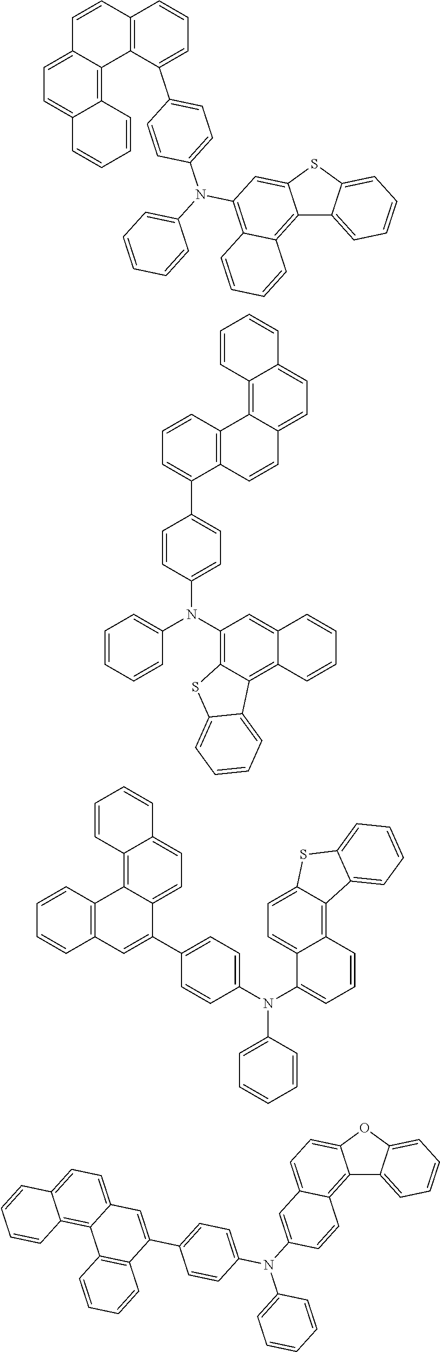

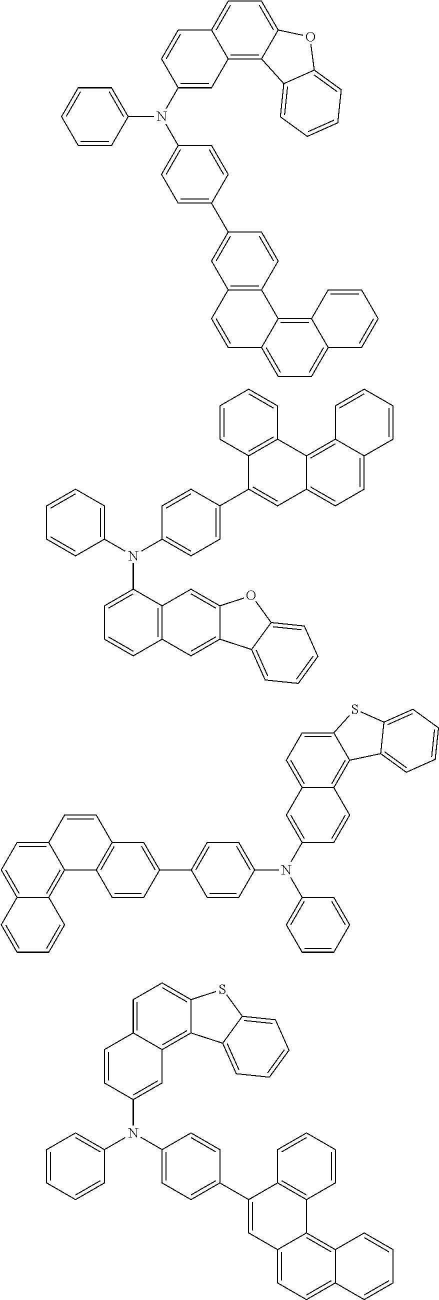

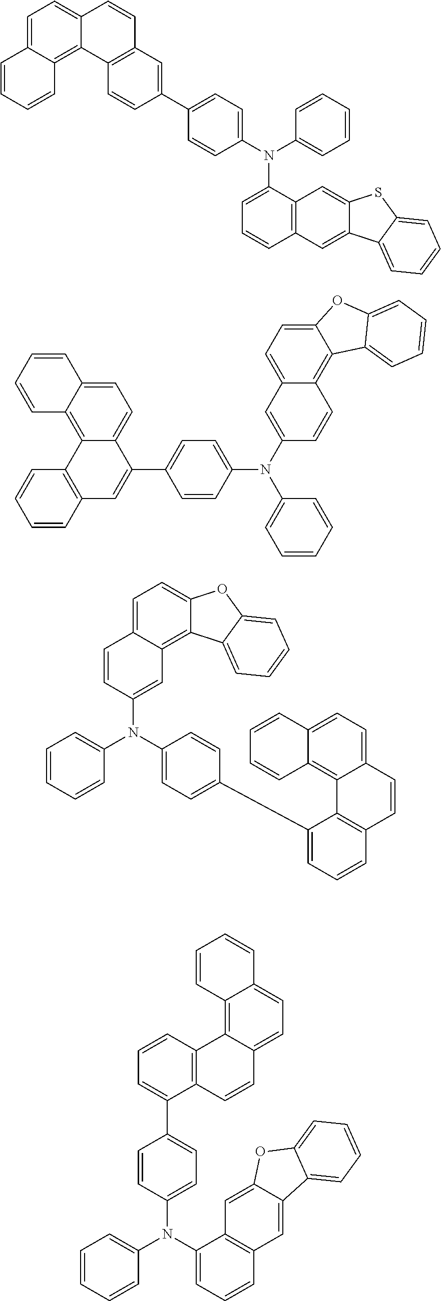

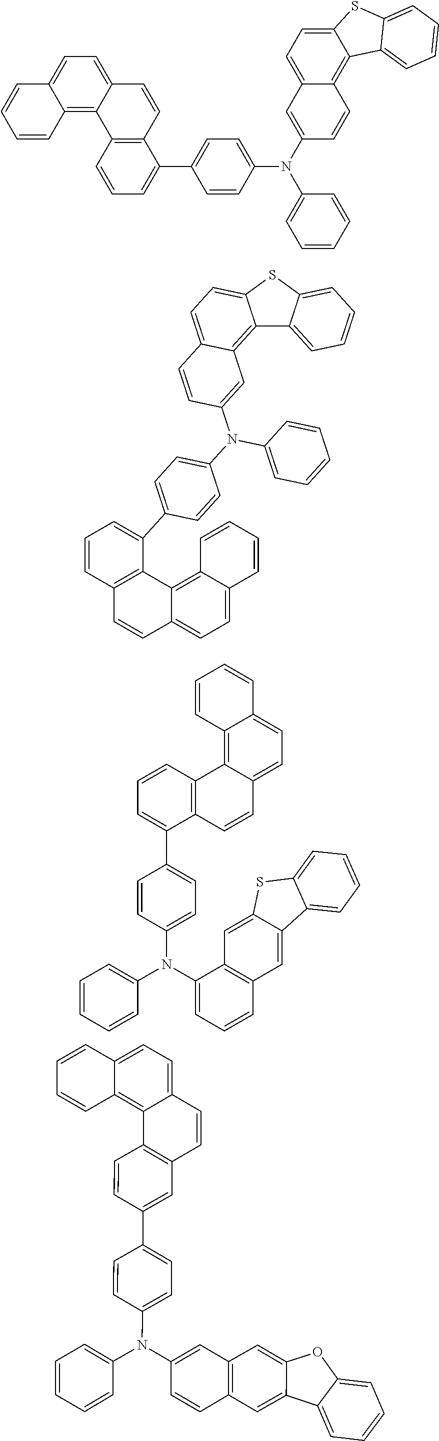

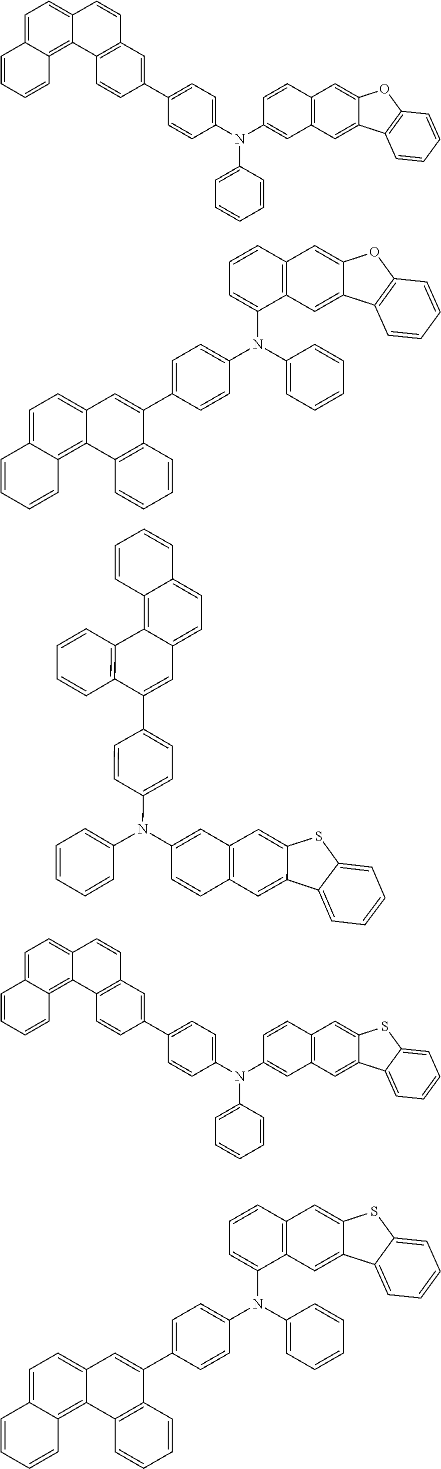

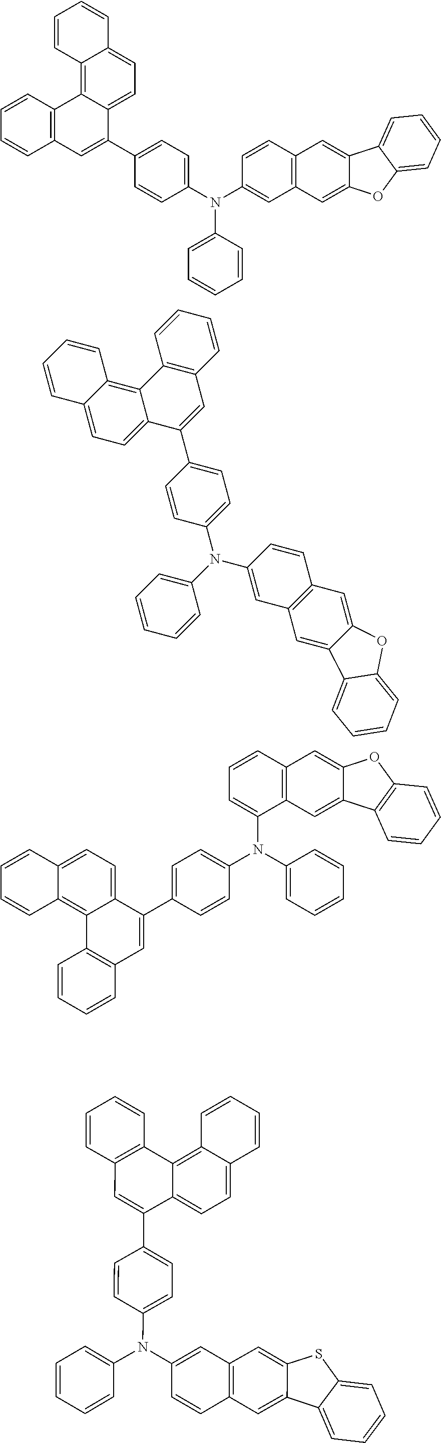

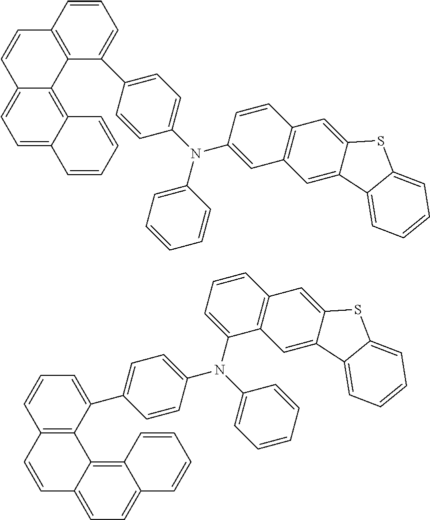

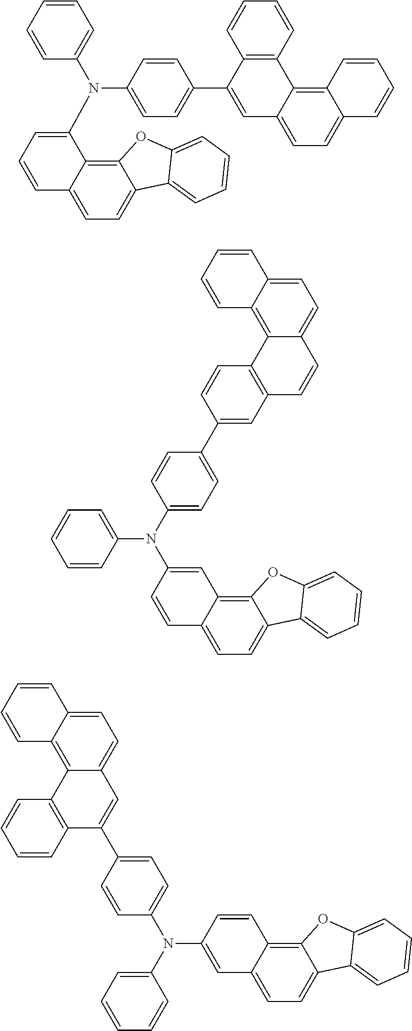 [SMILES: c1ccc(N(c2ccc(-c3cc4ccc5ccccc5c4c4ccccc34)cc2)c2cccc3ccc4c5ccccc5oc4c23)cc1 ; c1ccc(N(c2ccc(-c3ccc4c(ccc5ccc6ccccc6c54)c3)cc2)c2ccc3ccc4c5ccccc5oc4c3c2)cc1 ; c1ccc(N(c2ccc(-c3cc4ccc5ccccc5c4c4ccccc34)cc2)c2ccc3c(ccc4c5ccccc5oc34)c2)cc1]

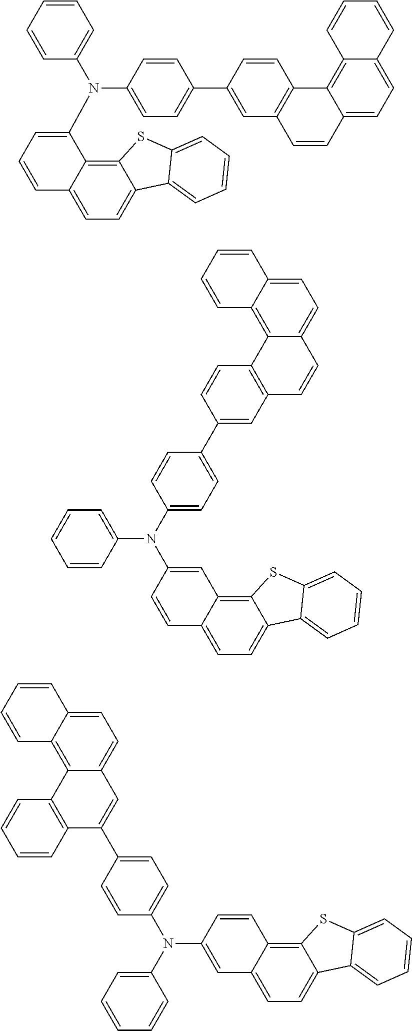 [SMILES: c1ccc(N(c2ccc(-c3ccc4c(ccc5ccc6ccccc6c54)c3)cc2)c2cccc3ccc4c5ccccc5sc4c23)cc1 ; c1ccc(N(c2ccc(-c3ccc4c(ccc5ccc6ccccc6c54)c3)cc2)c2ccc3ccc4c5ccccc5sc4c3c2)cc1 ; c1ccc(N(c2ccc(-c3cc4ccc5ccccc5c4c4ccccc34)cc2)c2ccc3c(ccc4c5ccccc5sc34)c2)cc1]

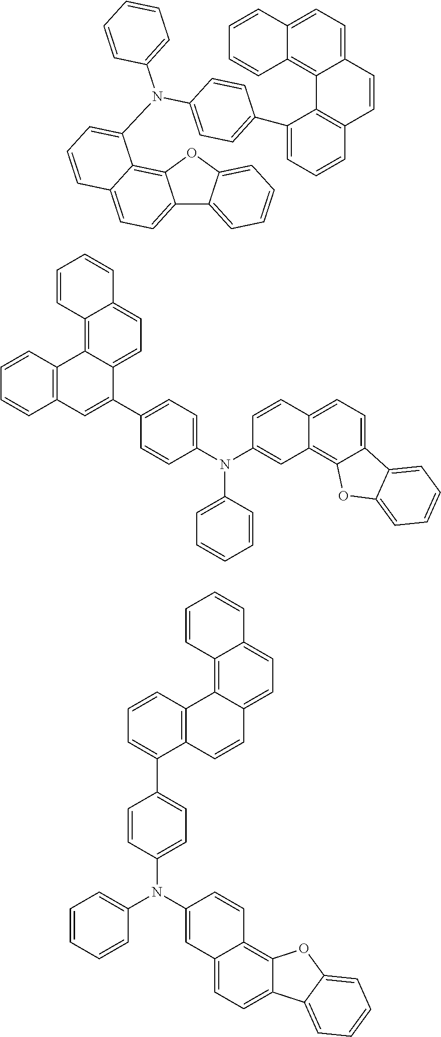

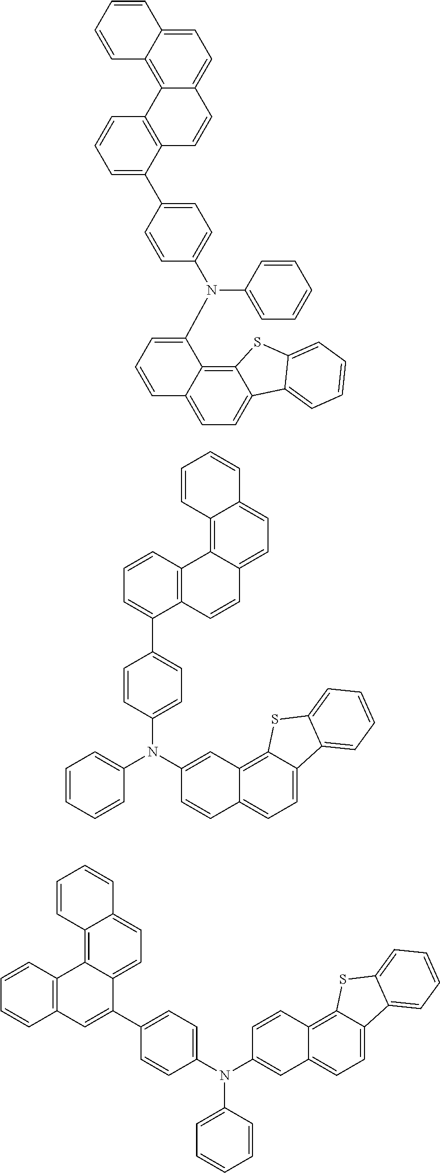

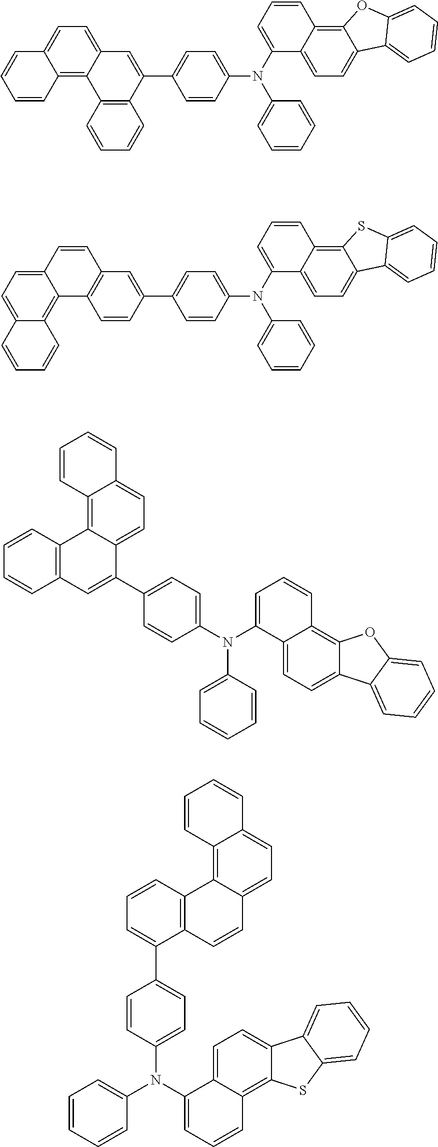 [SMILES: c1ccc(N(c2ccc(-c3cc4ccc5ccccc5c4c4ccccc34)cc2)c2cccc3c2ccc2c4ccccc4oc32)cc1 ; c1ccc(N(c2ccc(-c3ccc4c(ccc5ccc6ccccc6c54)c3)cc2)c2cccc3c2ccc2c4ccccc4sc32)cc1 ; c1ccc(N(c2ccc(-c3cc4ccccc4c4c3ccc3ccccc34)cc2)c2cccc3c2ccc2c4ccccc4oc32)cc1 ; c1ccc(N(c2ccc(-c3cccc4c3ccc3ccc5ccccc5c34)cc2)c2cccc3c2ccc2c4ccccc4sc32)cc1]

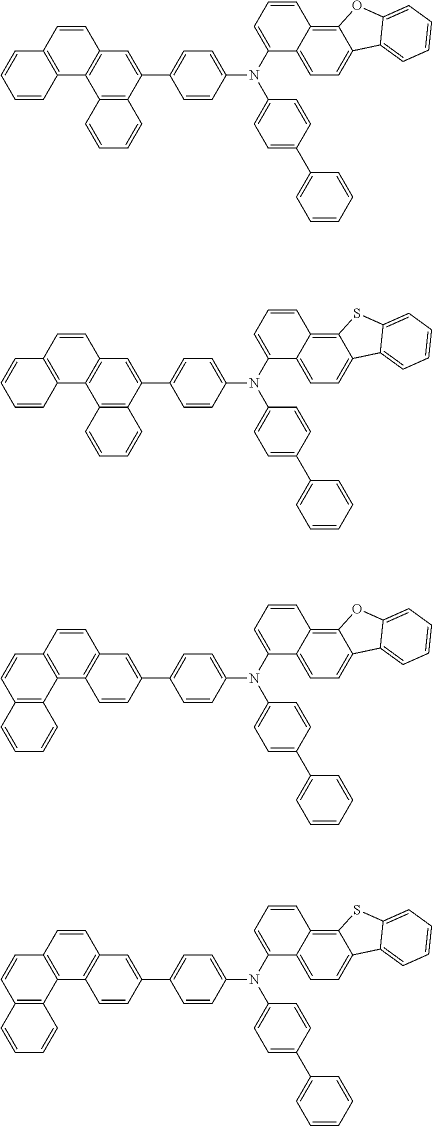 [SMILES: c1ccc(-c2ccc(N(c3ccc(-c4cc5ccc6ccccc6c5c5ccccc45)cc3)c3cccc4c3ccc3c5ccccc5oc43)cc2)cc1 ; c1ccc(-c2ccc(N(c3ccc(-c4cc5ccc6ccccc6c5c5ccccc45)cc3)c3cccc4c3ccc3c5ccccc5sc43)cc2)cc1 ; c1ccc(-c2ccc(N(c3ccc(-c4ccc5c(ccc6ccc7ccccc7c65)c4)cc3)c3cccc4c3ccc3c5ccccc5oc43)cc2)cc1 ; c1ccc(-c2ccc(N(c3ccc(-c4ccc5c(ccc6ccc7ccccc7c65)c4)cc3)c3cccc4c3ccc3c5ccccc5sc43)cc2)cc1]

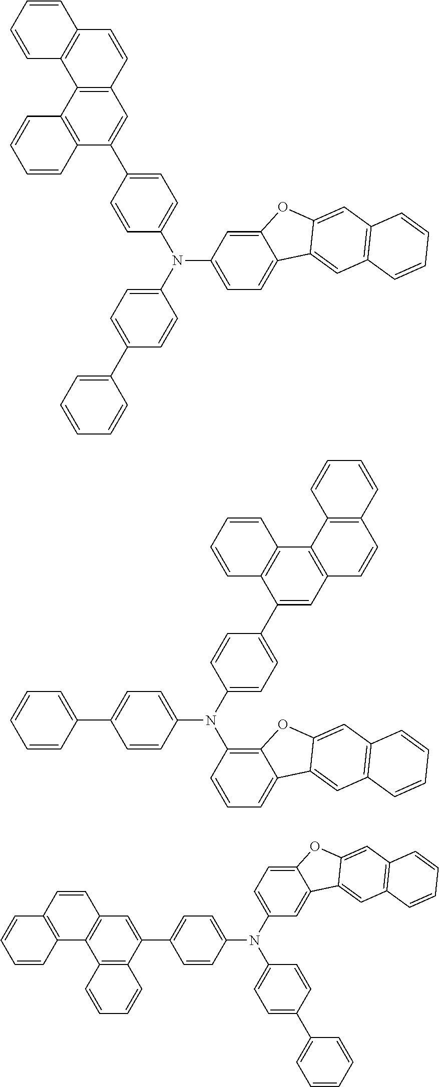 [SMILES: c1ccc(-c2ccc(N(c3ccc(-c4cc5ccc6ccccc6c5c5ccccc45)cc3)c3ccc4c(c3)oc3cc5ccccc5cc34)cc2)cc1 ; c1ccc(-c2ccc(N(c3ccc(-c4cc5ccc6ccccc6c5c5ccccc45)cc3)c3cccc4c3oc3cc5ccccc5cc34)cc2)cc1 ; c1ccc(-c2ccc(N(c3ccc(-c4cc5ccc6ccccc6c5c5ccccc45)cc3)c3ccc4oc5cc6ccccc6cc5c4c3)cc2)cc1]

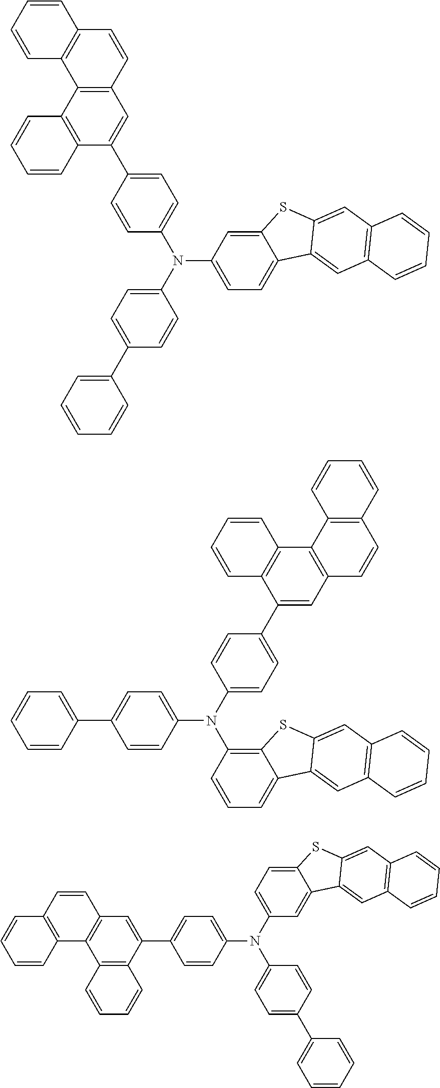 [SMILES: c1ccc(-c2ccc(N(c3ccc(-c4cc5ccc6ccccc6c5c5ccccc45)cc3)c3ccc4c(c3)sc3cc5ccccc5cc34)cc2)cc1 ; c1ccc(-c2ccc(N(c3ccc(-c4cc5ccc6ccccc6c5c5ccccc45)cc3)c3cccc4c3sc3cc5ccccc5cc34)cc2)cc1 ; c1ccc(-c2ccc(N(c3ccc(-c4cc5ccc6ccccc6c5c5ccccc45)cc3)c3ccc4sc5cc6ccccc6cc5c4c3)cc2)cc1]

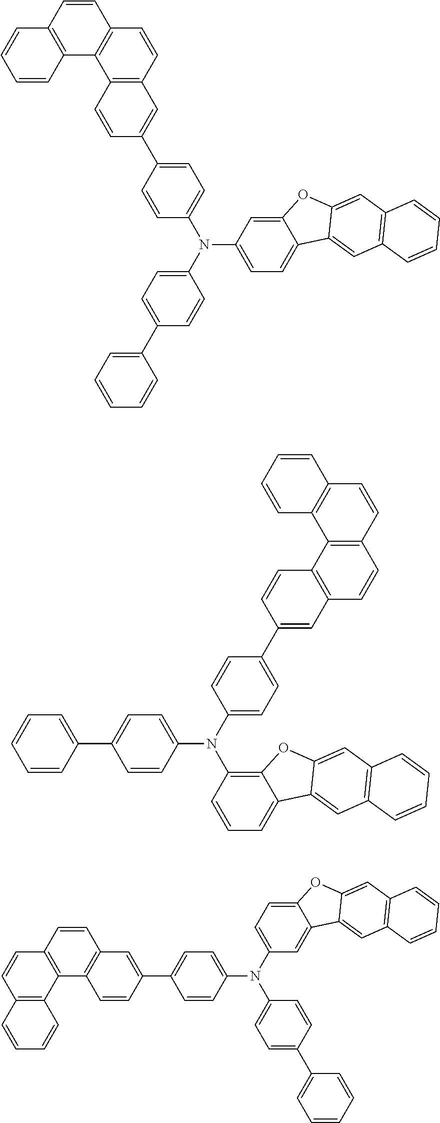 [SMILES: c1ccc(-c2ccc(N(c3ccc(-c4ccc5c(ccc6ccc7ccccc7c65)c4)cc3)c3ccc4c(c3)oc3cc5ccccc5cc34)cc2)cc1 ; c1ccc(-c2ccc(N(c3ccc(-c4ccc5c(ccc6ccc7ccccc7c65)c4)cc3)c3cccc4c3oc3cc5ccccc5cc34)cc2)cc1 ; c1ccc(-c2ccc(N(c3ccc(-c4ccc5c(ccc6ccc7ccccc7c65)c4)cc3)c3ccc4oc5cc6ccccc6cc5c4c3)cc2)cc1]

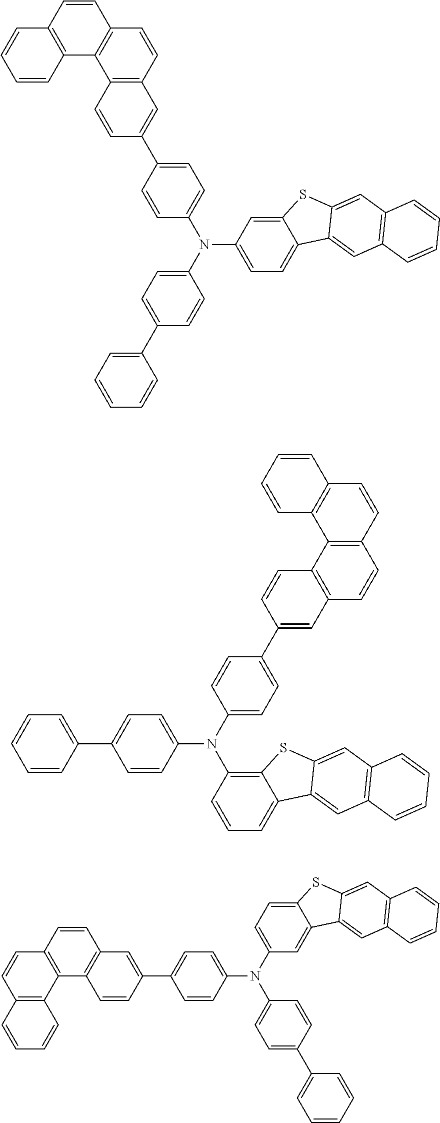 [SMILES: c1ccc(-c2ccc(N(c3ccc(-c4ccc5c(ccc6ccc7ccccc7c65)c4)cc3)c3ccc4c(c3)sc3cc5ccccc5cc34)cc2)cc1 ; c1ccc(-c2ccc(N(c3ccc(-c4ccc5c(ccc6ccc7ccccc7c65)c4)cc3)c3cccc4c3sc3cc5ccccc5cc34)cc2)cc1 ; c1ccc(-c2ccc(N(c3ccc(-c4ccc5c(ccc6ccc7ccccc7c65)c4)cc3)c3ccc4sc5cc6ccccc6cc5c4c3)cc2)cc1]

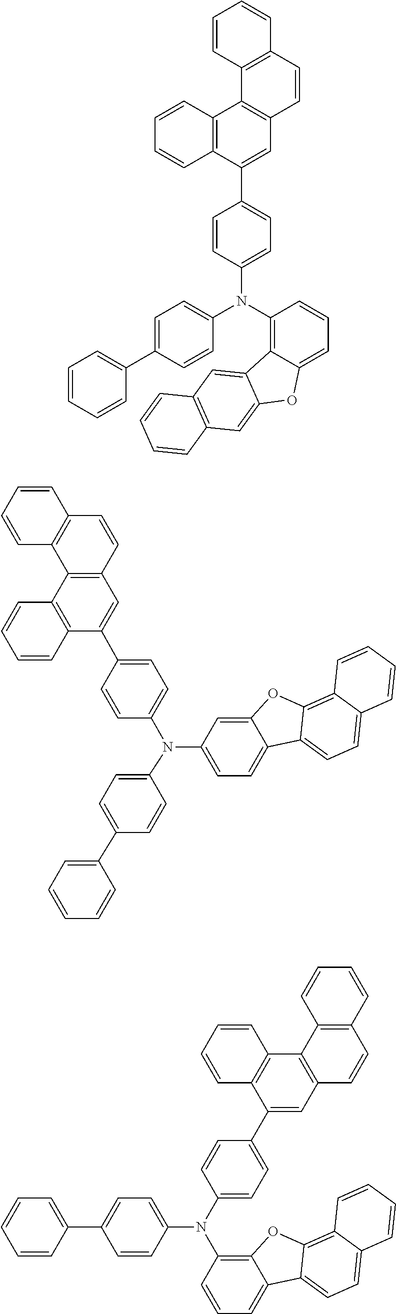 [SMILES: c1ccc(-c2ccc(N(c3ccc(-c4cc5ccc6ccccc6c5c5ccccc45)cc3)c3cccc4oc5cc6ccccc6cc5c34)cc2)cc1 ; c1ccc(-c2ccc(N(c3ccc(-c4cc5ccc6ccccc6c5c5ccccc45)cc3)c3ccc4c(c3)oc3c5ccccc5ccc43)cc2)cc1 ; c1ccc(-c2ccc(N(c3ccc(-c4cc5ccc6ccccc6c5c5ccccc45)cc3)c3cccc4c3oc3c5ccccc5ccc43)cc2)cc1]

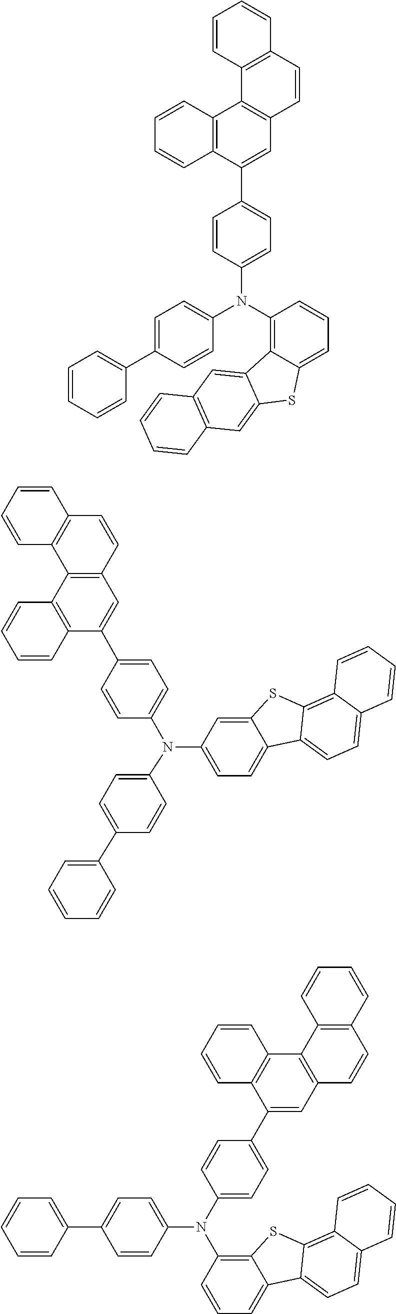 [SMILES: c1ccc(-c2ccc(N(c3ccc(-c4cc5ccc6ccccc6c5c5ccccc45)cc3)c3cccc4sc5cc6ccccc6cc5c34)cc2)cc1 ; c1ccc(-c2ccc(N(c3ccc(-c4cc5ccc6ccccc6c5c5ccccc45)cc3)c3ccc4c(c3)sc3c5ccccc5ccc43)cc2)cc1 ; c1ccc(-c2ccc(N(c3ccc(-c4cc5ccc6ccccc6c5c5ccccc45)cc3)c3cccc4c3sc3c5ccccc5ccc43)cc2)cc1]

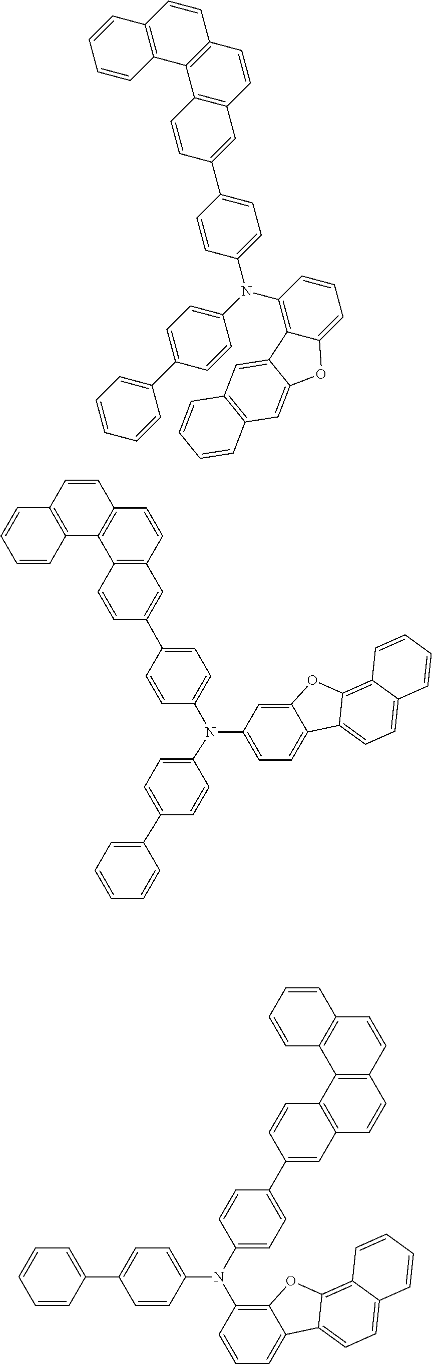 [SMILES: c1ccc(-c2ccc(N(c3ccc(-c4ccc5c(ccc6ccc7ccccc7c65)c4)cc3)c3cccc4oc5cc6ccccc6cc5c34)cc2)cc1 ; c1ccc(-c2ccc(N(c3ccc(-c4ccc5c(ccc6ccc7ccccc7c65)c4)cc3)c3ccc4c(c3)oc3c5ccccc5ccc43)cc2)cc1 ; c1ccc(-c2ccc(N(c3ccc(-c4ccc5c(ccc6ccc7ccccc7c65)c4)cc3)c3cccc4c3oc3c5ccccc5ccc43)cc2)cc1]

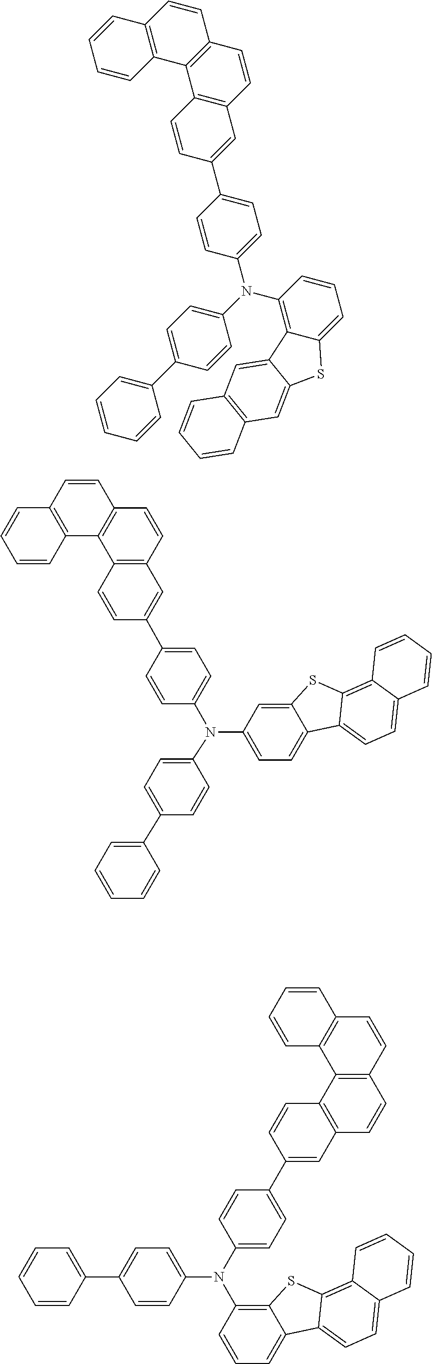 [SMILES: c1ccc(-c2ccc(N(c3ccc(-c4ccc5c(ccc6ccc7ccccc7c65)c4)cc3)c3cccc4sc5cc6ccccc6cc5c34)cc2)cc1 ; c1ccc(-c2ccc(N(c3ccc(-c4ccc5c(ccc6ccc7ccccc7c65)c4)cc3)c3ccc4c(c3)sc3c5ccccc5ccc43)cc2)cc1 ; c1ccc(-c2ccc(N(c3ccc(-c4ccc5c(ccc6ccc7ccccc7c65)c4)cc3)c3cccc4c3sc3c5ccccc5ccc43)cc2)cc1]

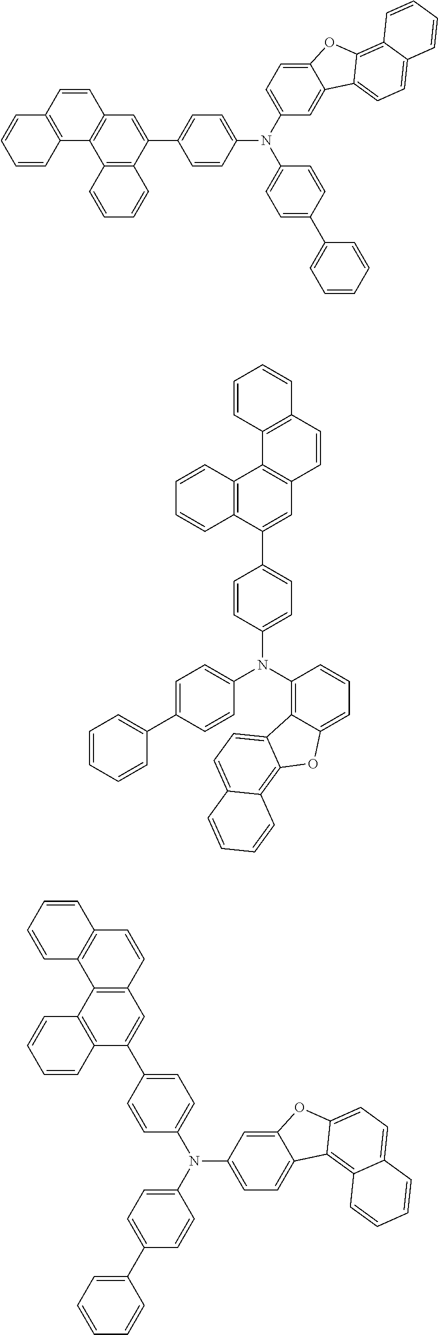 [SMILES: c1ccc(-c2ccc(N(c3ccc(-c4cc5ccc6ccccc6c5c5ccccc45)cc3)c3ccc4oc5c6ccccc6ccc5c4c3)cc2)cc1 ; c1ccc(-c2ccc(N(c3ccc(-c4cc5ccc6ccccc6c5c5ccccc45)cc3)c3cccc4oc5c6ccccc6ccc5c34)cc2)cc1 ; c1ccc(-c2ccc(N(c3ccc(-c4cc5ccc6ccccc6c5c5ccccc45)cc3)c3ccc4c(c3)oc3ccc5ccccc5c34)cc2)cc1]

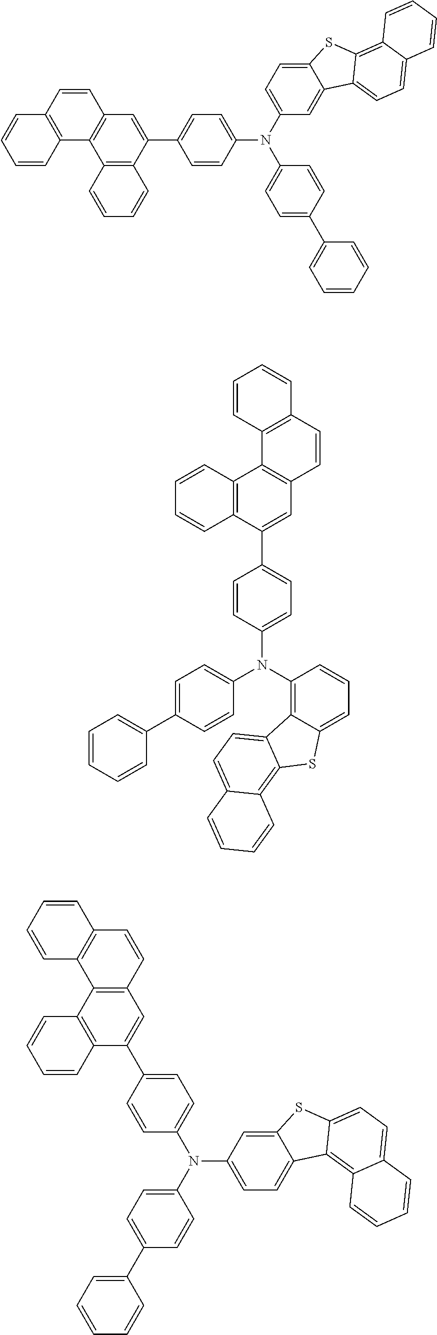 [SMILES: c1ccc(-c2ccc(N(c3ccc(-c4cc5ccc6ccccc6c5c5ccccc45)cc3)c3ccc4sc5c6ccccc6ccc5c4c3)cc2)cc1 ; c1ccc(-c2ccc(N(c3ccc(-c4cc5ccc6ccccc6c5c5ccccc45)cc3)c3cccc4sc5c6ccccc6ccc5c34)cc2)cc1 ; c1ccc(-c2ccc(N(c3ccc(-c4cc5ccc6ccccc6c5c5ccccc45)cc3)c3ccc4c(c3)sc3ccc5ccccc5c34)cc2)cc1]

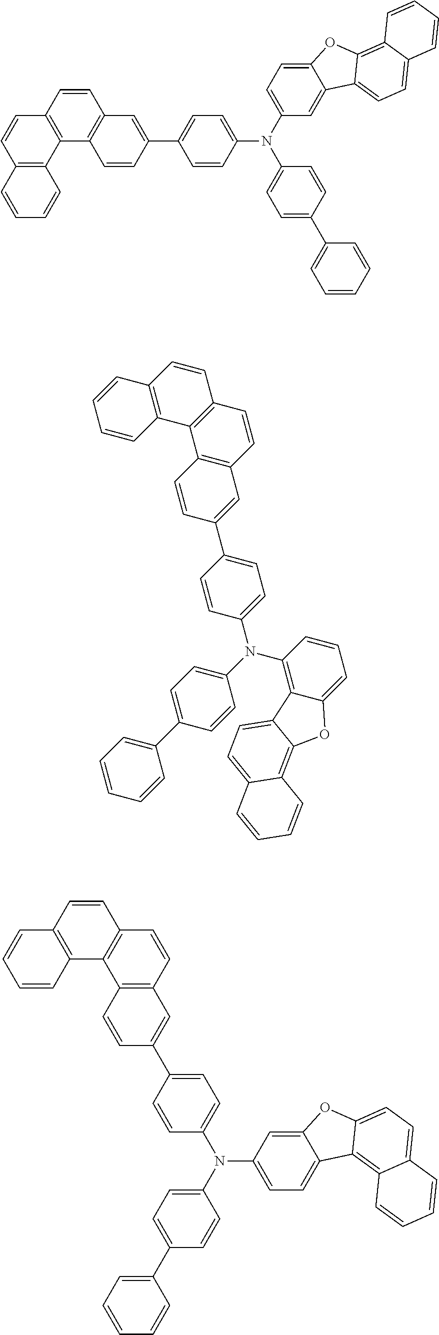 [SMILES: c1ccc(-c2ccc(N(c3ccc(-c4ccc5c(ccc6ccc7ccccc7c65)c4)cc3)c3ccc4oc5c6ccccc6ccc5c4c3)cc2)cc1 ; c1ccc(-c2ccc(N(c3ccc(-c4ccc5c(ccc6ccc7ccccc7c65)c4)cc3)c3cccc4oc5c6ccccc6ccc5c34)cc2)cc1 ; c1ccc(-c2ccc(N(c3ccc(-c4ccc5c(ccc6ccc7ccccc7c65)c4)cc3)c3ccc4c(c3)oc3ccc5ccccc5c34)cc2)cc1]

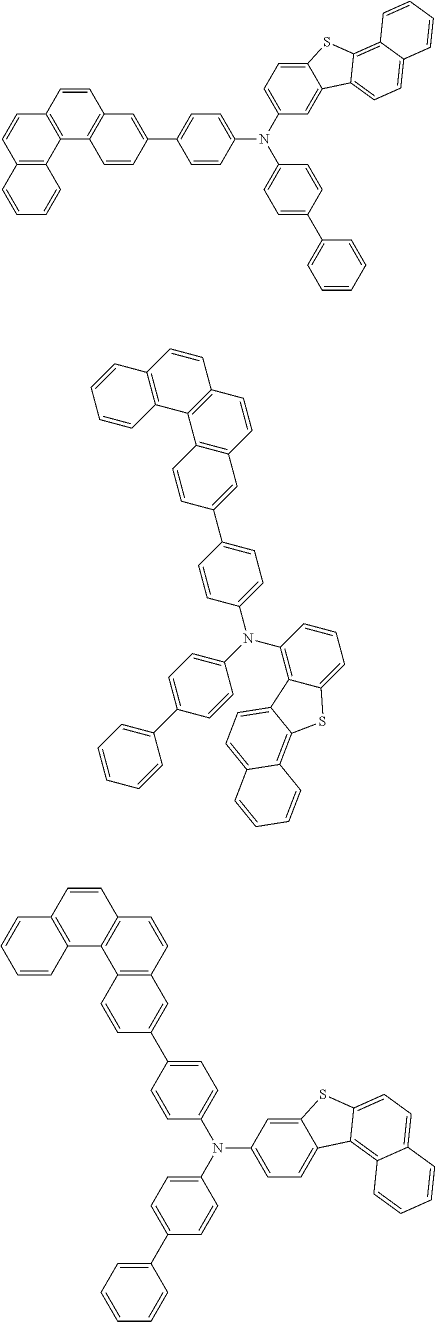 [SMILES: c1ccc(-c2ccc(N(c3ccc(-c4ccc5c(ccc6ccc7ccccc7c65)c4)cc3)c3ccc4sc5c6ccccc6ccc5c4c3)cc2)cc1 ; c1ccc(-c2ccc(N(c3ccc(-c4ccc5c(ccc6ccc7ccccc7c65)c4)cc3)c3cccc4sc5c6ccccc6ccc5c34)cc2)cc1 ; c1ccc(-c2ccc(N(c3ccc(-c4ccc5c(ccc6ccc7ccccc7c65)c4)cc3)c3ccc4c(c3)sc3ccc5ccccc5c34)cc2)cc1]

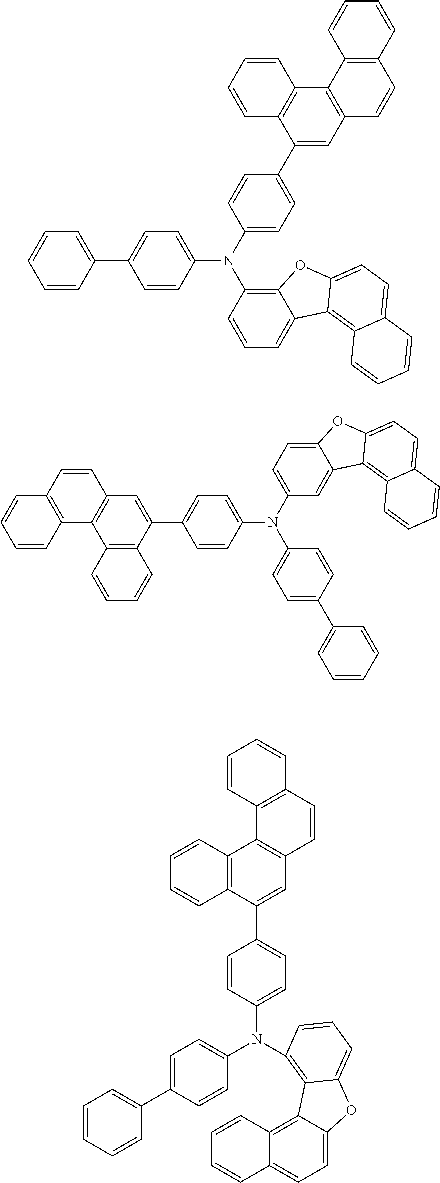

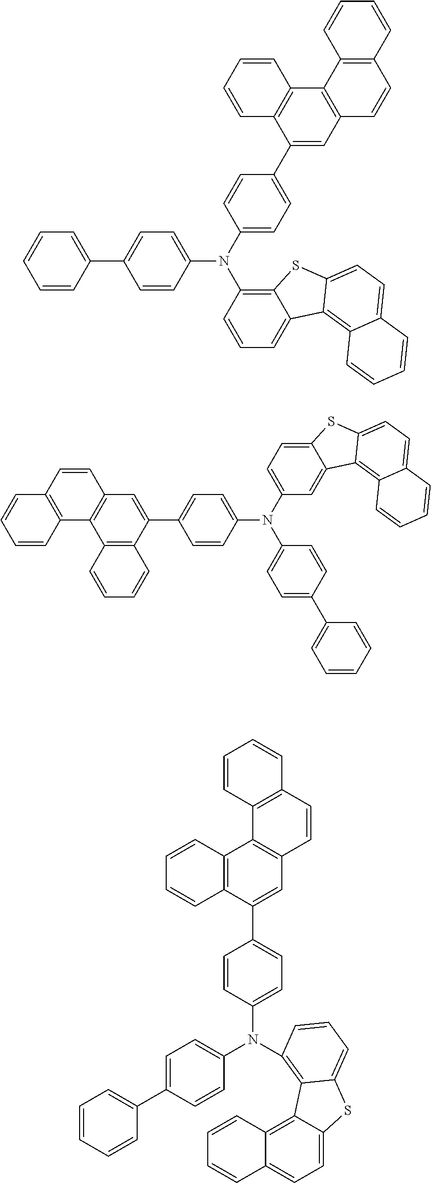

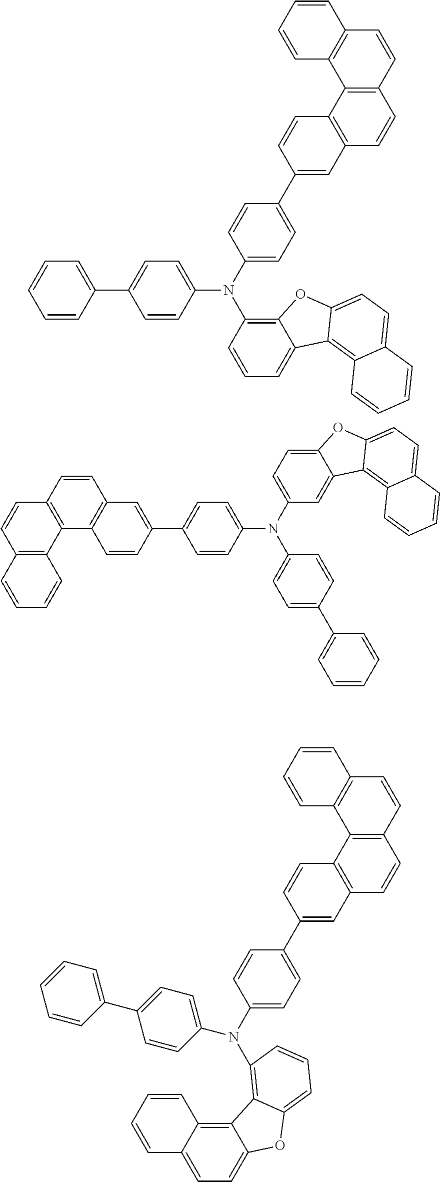

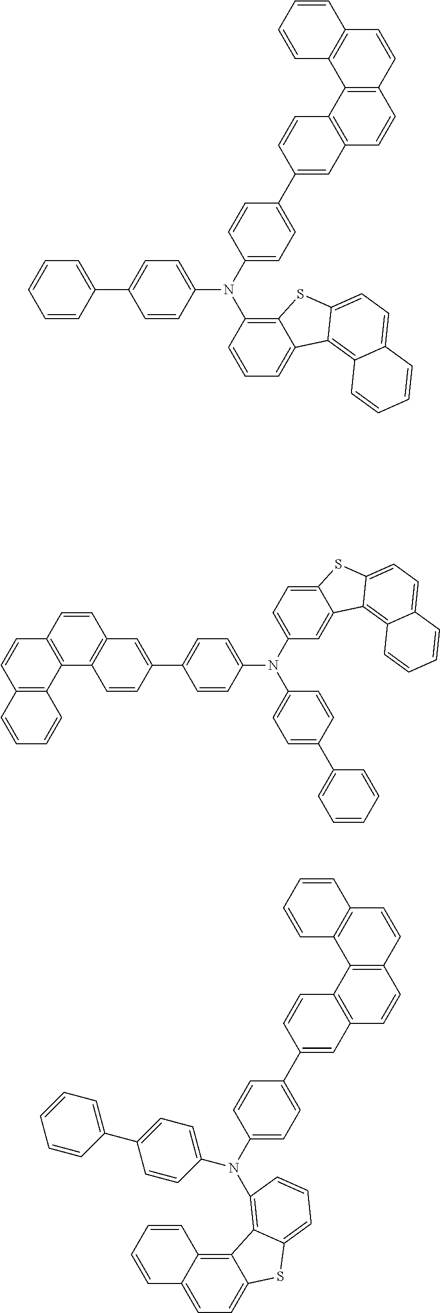 [SMILES: c1ccc(-c2ccc(N(c3ccc(-c4ccc5c(ccc6ccc7ccccc7c65)c4)cc3)c3cccc4c3sc3ccc5ccccc5c34)cc2)cc1 ; c1ccc(-c2ccc(N(c3ccc(-c4ccc5c(ccc6ccc7ccccc7c65)c4)cc3)c3ccc4sc5ccc6ccccc6c5c4c3)cc2)cc1 ; c1ccc(-c2ccc(N(c3ccc(-c4ccc5c(ccc6ccc7ccccc7c65)c4)cc3)c3cccc4sc5ccc6ccccc6c5c34)cc2)cc1]

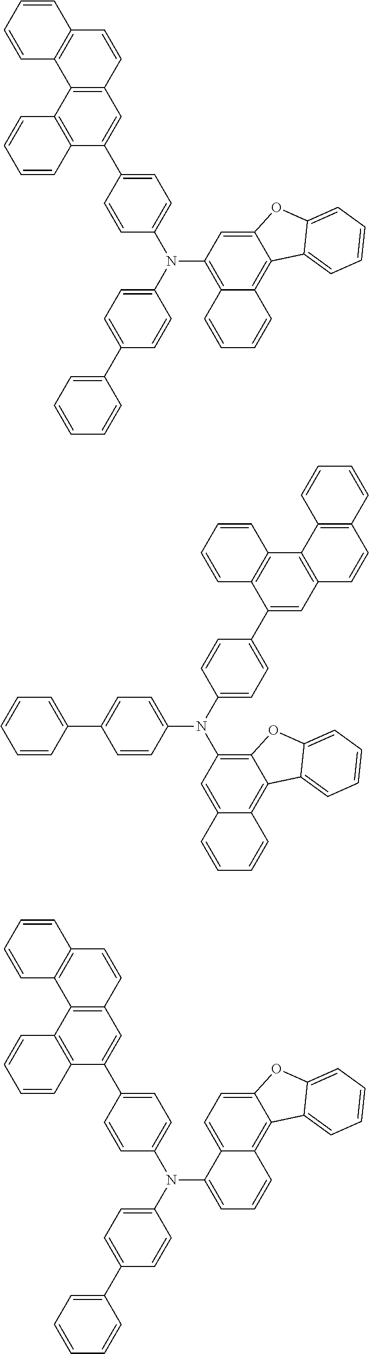 [SMILES: c1ccc(-c2ccc(N(c3ccc(-c4cc5ccc6ccccc6c5c5ccccc45)cc3)c3cc4oc5ccccc5c4c4ccccc34)cc2)cc1 ; c1ccc(-c2ccc(N(c3ccc(-c4cc5ccc6ccccc6c5c5ccccc45)cc3)c3cc4ccccc4c4c3oc3ccccc34)cc2)cc1 ; c1ccc(-c2ccc(N(c3ccc(-c4cc5ccc6ccccc6c5c5ccccc45)cc3)c3cccc4c3ccc3oc5ccccc5c34)cc2)cc1]

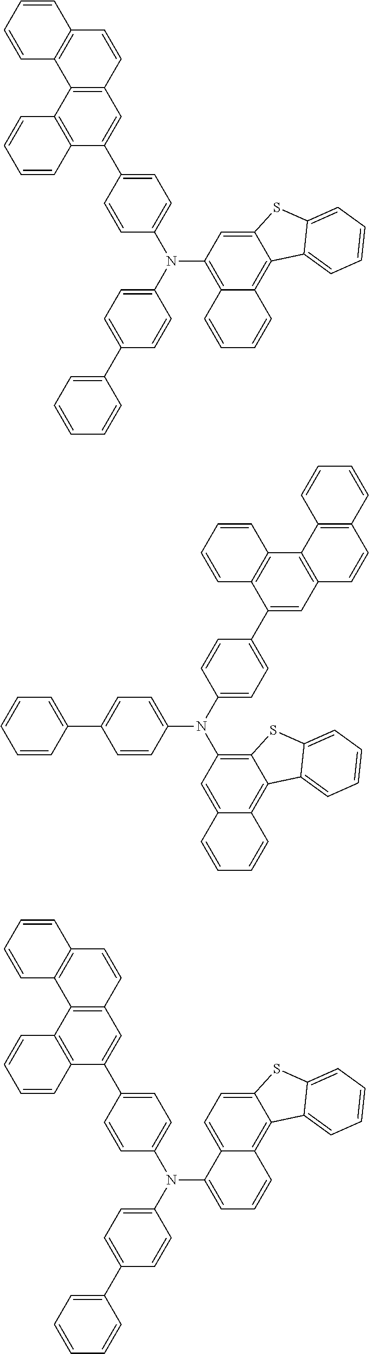 [SMILES: c1ccc(-c2ccc(N(c3ccc(-c4cc5ccc6ccccc6c5c5ccccc45)cc3)c3cc4sc5ccccc5c4c4ccccc34)cc2)cc1 ; c1ccc(-c2ccc(N(c3ccc(-c4cc5ccc6ccccc6c5c5ccccc45)cc3)c3cc4ccccc4c4c3sc3ccccc34)cc2)cc1 ; c1ccc(-c2ccc(N(c3ccc(-c4cc5ccc6ccccc6c5c5ccccc45)cc3)c3cccc4c3ccc3sc5ccccc5c34)cc2)cc1]

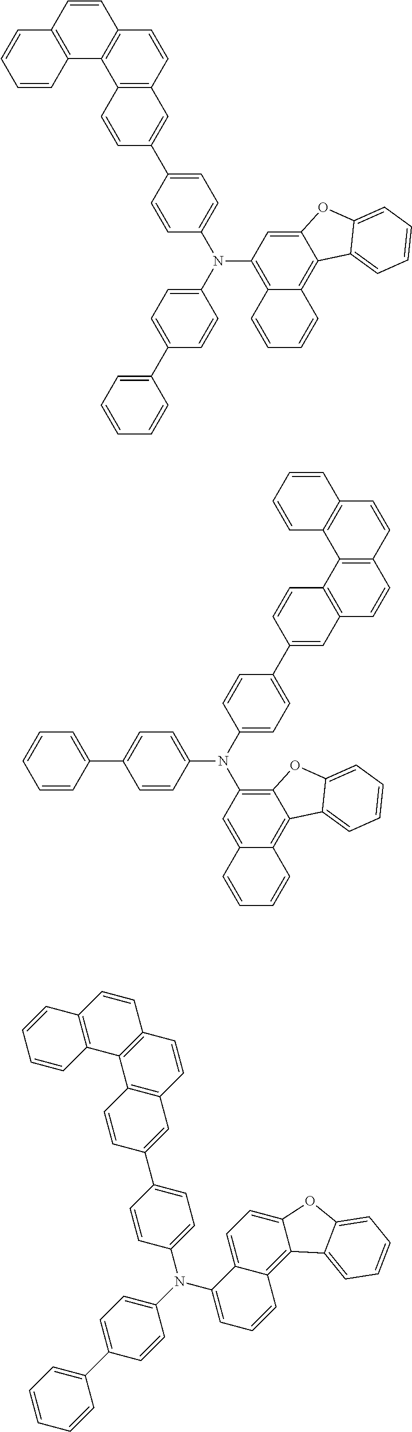 [SMILES: c1ccc(-c2ccc(N(c3ccc(-c4ccc5c(ccc6ccc7ccccc7c65)c4)cc3)c3cc4oc5ccccc5c4c4ccccc34)cc2)cc1 ; c1ccc(-c2ccc(N(c3ccc(-c4ccc5c(ccc6ccc7ccccc7c65)c4)cc3)c3cc4ccccc4c4c3oc3ccccc34)cc2)cc1 ; c1ccc(-c2ccc(N(c3ccc(-c4ccc5c(ccc6ccc7ccccc7c65)c4)cc3)c3cccc4c3ccc3oc5ccccc5c34)cc2)cc1]

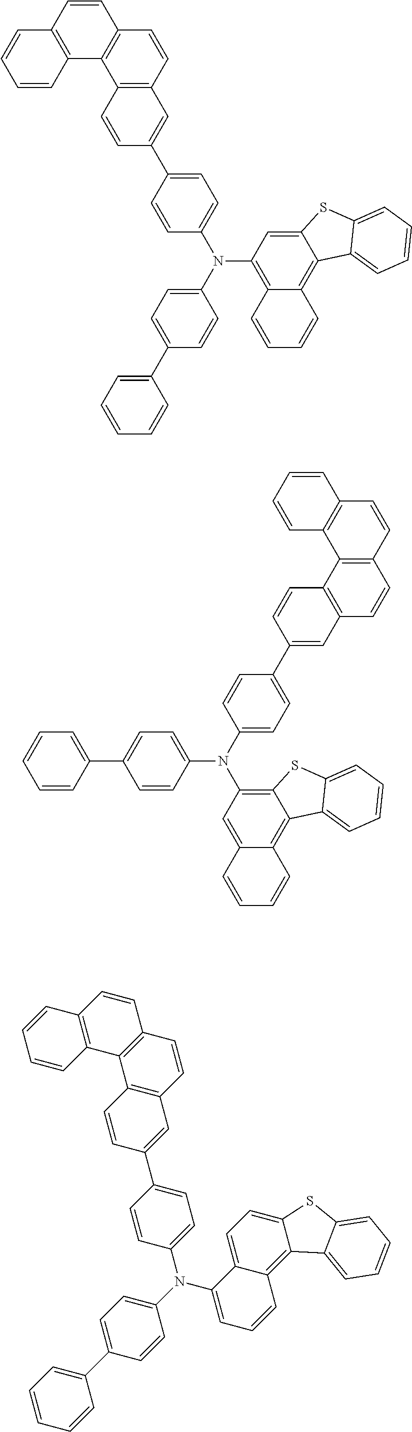 [SMILES: c1ccc(-c2ccc(N(c3ccc(-c4ccc5c(ccc6ccc7ccccc7c65)c4)cc3)c3cc4sc5ccccc5c4c4ccccc34)cc2)cc1 ; c1ccc(-c2ccc(N(c3ccc(-c4ccc5c(ccc6ccc7ccccc7c65)c4)cc3)c3cc4ccccc4c4c3sc3ccccc34)cc2)cc1 ; c1ccc(-c2ccc(N(c3ccc(-c4ccc5c(ccc6ccc7ccccc7c65)c4)cc3)c3cccc4c3ccc3sc5ccccc5c34)cc2)cc1]

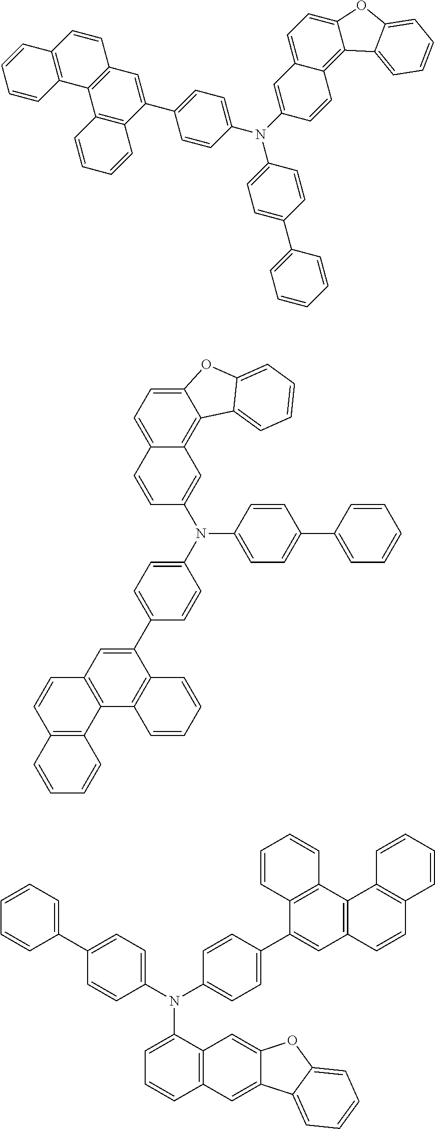 [SMILES: c1ccc(-c2ccc(N(c3ccc(-c4cc5ccc6ccccc6c5c5ccccc45)cc3)c3ccc4c(ccc5oc6ccccc6c54)c3)cc2)cc1 ; c1ccc(-c2ccc(N(c3ccc(-c4cc5ccc6ccccc6c5c5ccccc45)cc3)c3ccc4ccc5oc6ccccc6c5c4c3)cc2)cc1 ; c1ccc(-c2ccc(N(c3ccc(-c4cc5ccc6ccccc6c5c5ccccc45)cc3)c3cccc4cc5c(cc34)oc3ccccc35)cc2)cc1]

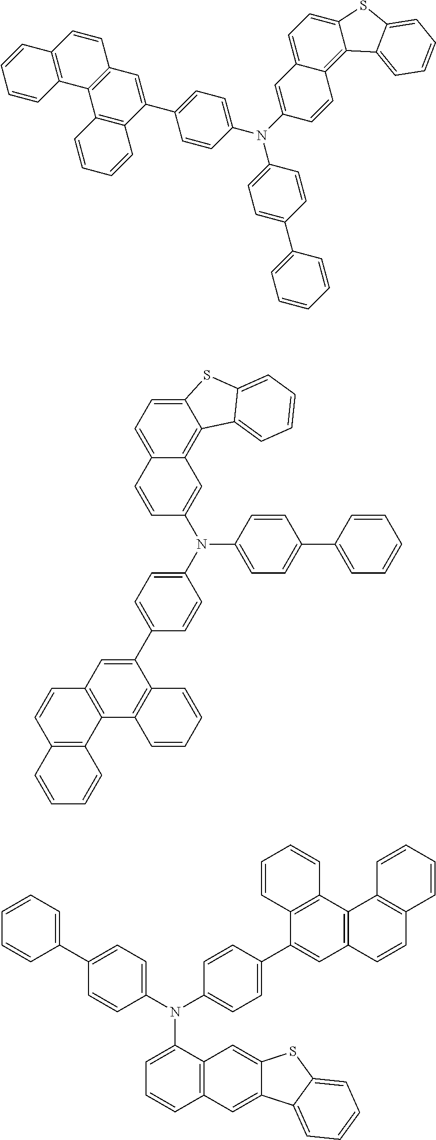 [SMILES: c1ccc(-c2ccc(N(c3ccc(-c4cc5ccc6ccccc6c5c5ccccc45)cc3)c3ccc4c(ccc5sc6ccccc6c54)c3)cc2)cc1 ; c1ccc(-c2ccc(N(c3ccc(-c4cc5ccc6ccccc6c5c5ccccc45)cc3)c3ccc4ccc5sc6ccccc6c5c4c3)cc2)cc1 ; c1ccc(-c2ccc(N(c3ccc(-c4cc5ccc6ccccc6c5c5ccccc45)cc3)c3cccc4cc5c(cc34)sc3ccccc35)cc2)cc1]

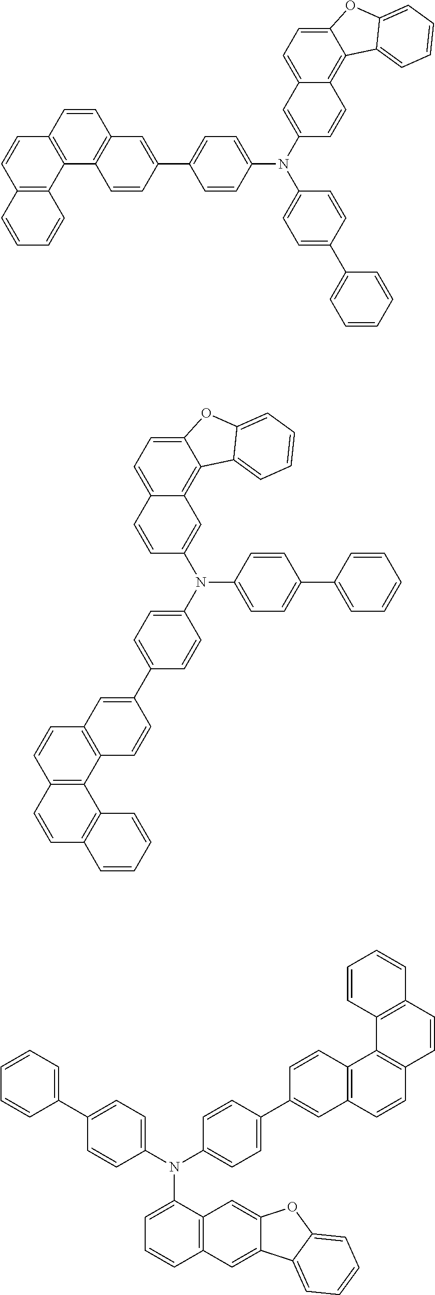 [SMILES: c1ccc(-c2ccc(N(c3ccc(-c4ccc5c(ccc6ccc7ccccc7c65)c4)cc3)c3ccc4c(ccc5oc6ccccc6c54)c3)cc2)cc1 ; c1ccc(-c2ccc(N(c3ccc(-c4ccc5c(ccc6ccc7ccccc7c65)c4)cc3)c3ccc4ccc5oc6ccccc6c5c4c3)cc2)cc1 ; c1ccc(-c2ccc(N(c3ccc(-c4ccc5c(ccc6ccc7ccccc7c65)c4)cc3)c3cccc4cc5c(cc34)oc3ccccc35)cc2)cc1]

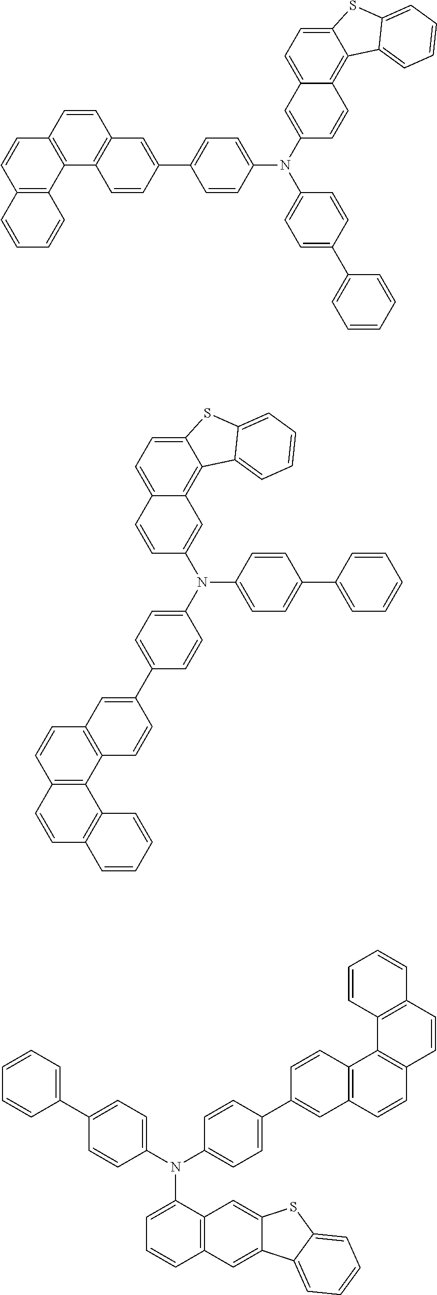 [SMILES: c1ccc(-c2ccc(N(c3ccc(-c4ccc5c(ccc6ccc7ccccc7c65)c4)cc3)c3ccc4c(ccc5sc6ccccc6c54)c3)cc2)cc1 ; c1ccc(-c2ccc(N(c3ccc(-c4ccc5c(ccc6ccc7ccccc7c65)c4)cc3)c3ccc4ccc5sc6ccccc6c5c4c3)cc2)cc1 ; c1ccc(-c2ccc(N(c3ccc(-c4ccc5c(ccc6ccc7ccccc7c65)c4)cc3)c3cccc4cc5c(cc34)sc3ccccc35)cc2)cc1]

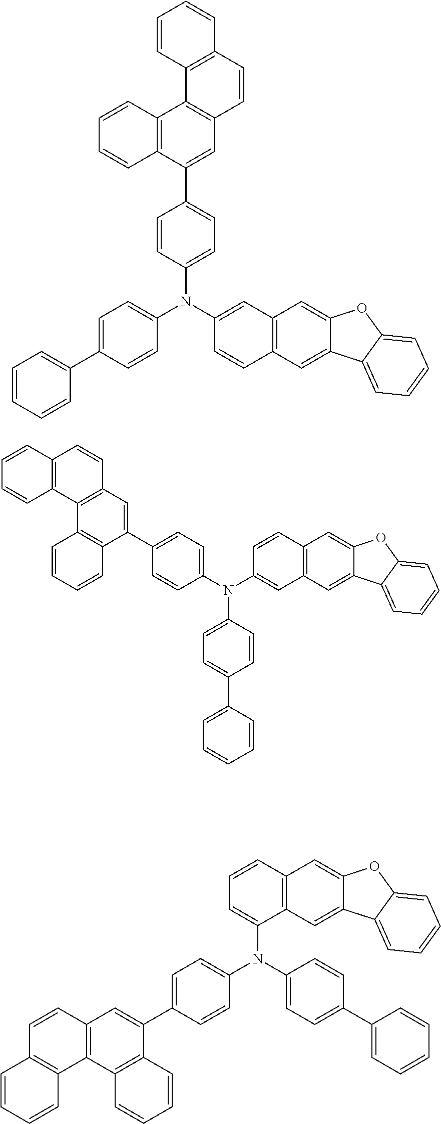

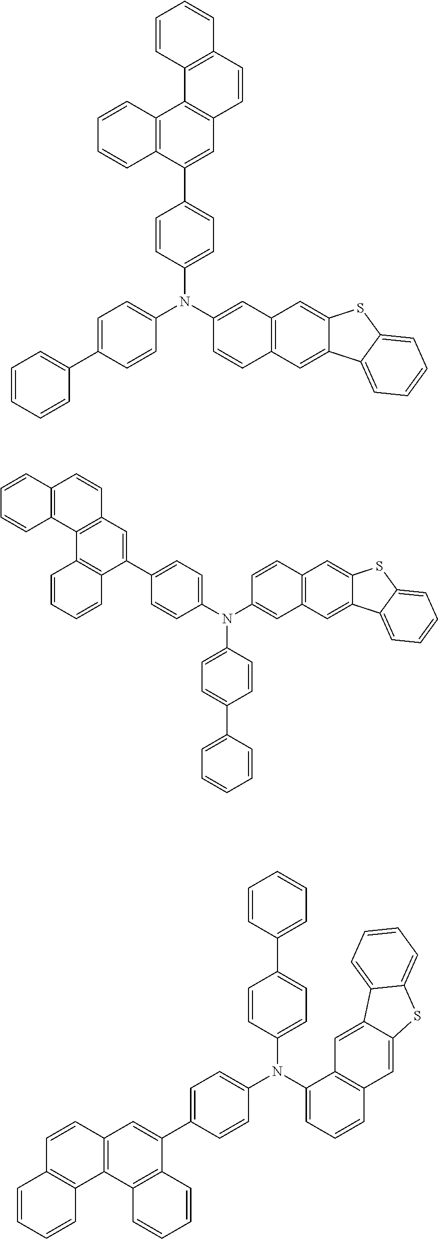 [SMILES: c1ccc(-c2ccc(N(c3ccc(-c4cc5ccc6ccccc6c5c5ccccc45)cc3)c3ccc4cc5c(cc4c3)sc3ccccc35)cc2)cc1 ; c1ccc(-c2ccc(N(c3ccc(-c4cc5ccc6ccccc6c5c5ccccc45)cc3)c3ccc4cc5sc6ccccc6c5cc4c3)cc2)cc1 ; c1ccc(-c2ccc(N(c3ccc(-c4cc5ccc6ccccc6c5c5ccccc45)cc3)c3cccc4cc5sc6ccccc6c5cc34)cc2)cc1]

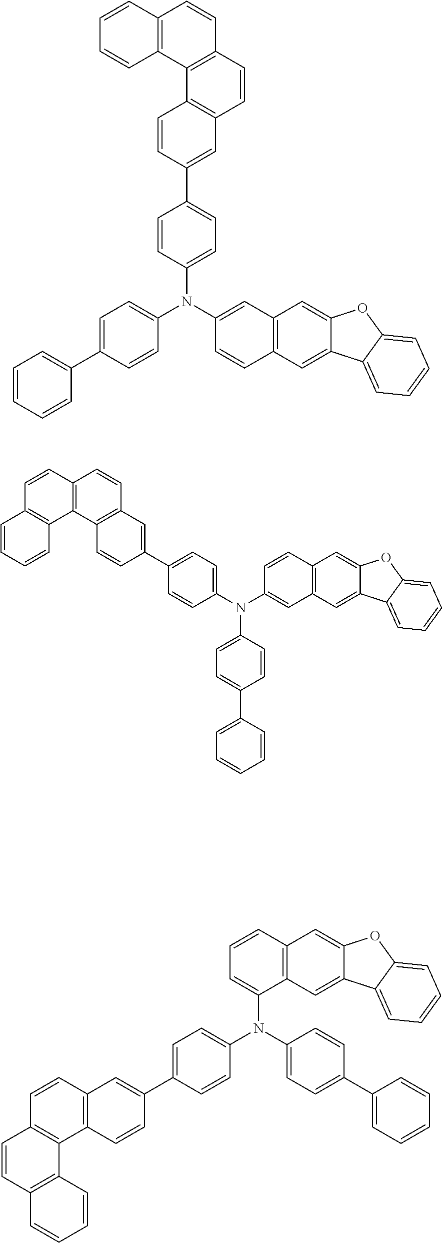 [SMILES: c1ccc(-c2ccc(N(c3ccc(-c4ccc5c(ccc6ccc7ccccc7c65)c4)cc3)c3ccc4cc5c(cc4c3)oc3ccccc35)cc2)cc1 ; c1ccc(-c2ccc(N(c3ccc(-c4ccc5c(ccc6ccc7ccccc7c65)c4)cc3)c3ccc4cc5oc6ccccc6c5cc4c3)cc2)cc1 ; c1ccc(-c2ccc(N(c3ccc(-c4ccc5c(ccc6ccc7ccccc7c65)c4)cc3)c3cccc4cc5oc6ccccc6c5cc34)cc2)cc1]

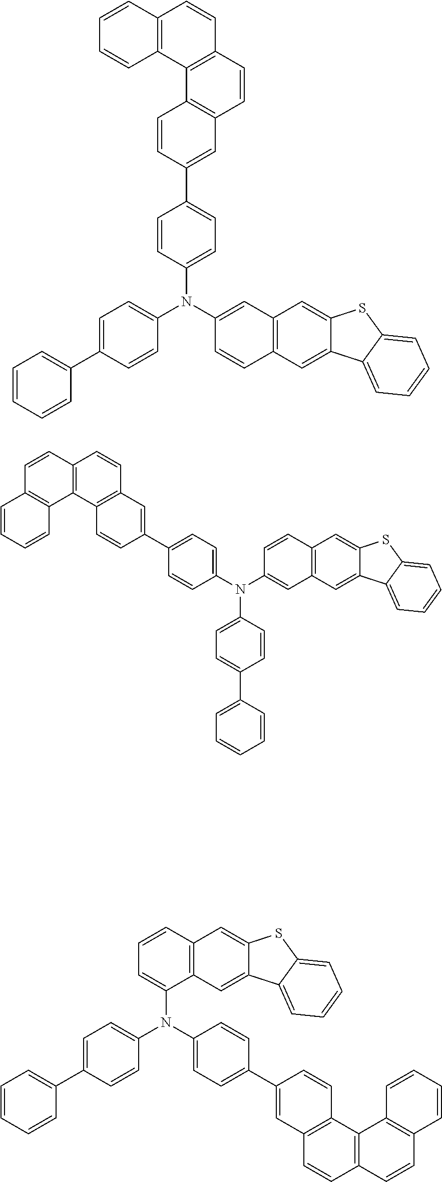

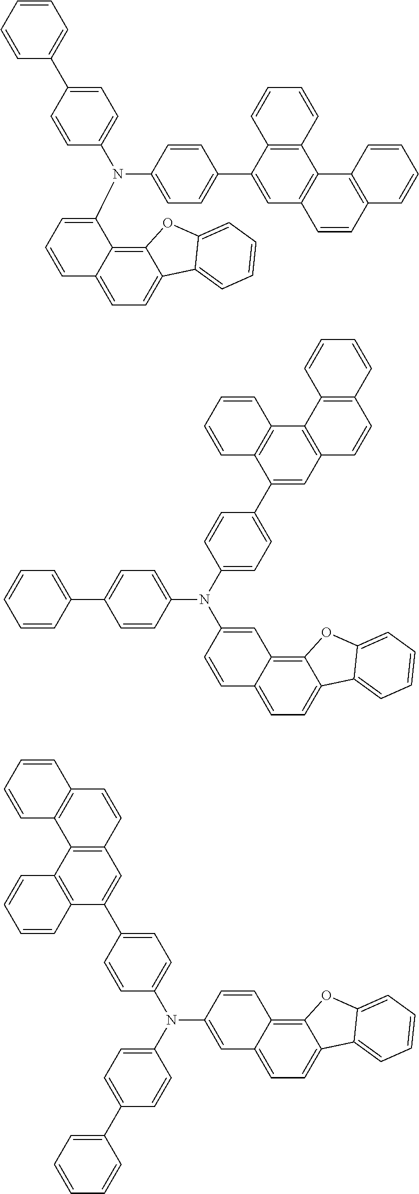 [SMILES: c1ccc(-c2ccc(N(c3ccc(-c4cc5ccc6ccccc6c5c5ccccc45)cc3)c3cccc4ccc5c6ccccc6oc5c34)cc2)cc1 ; c1ccc(-c2ccc(N(c3ccc(-c4cc5ccc6ccccc6c5c5ccccc45)cc3)c3ccc4ccc5c6ccccc6oc5c4c3)cc2)cc1 ; c1ccc(-c2ccc(N(c3ccc(-c4cc5ccc6ccccc6c5c5ccccc45)cc3)c3ccc4c(ccc5c6ccccc6oc45)c3)cc2)cc1]

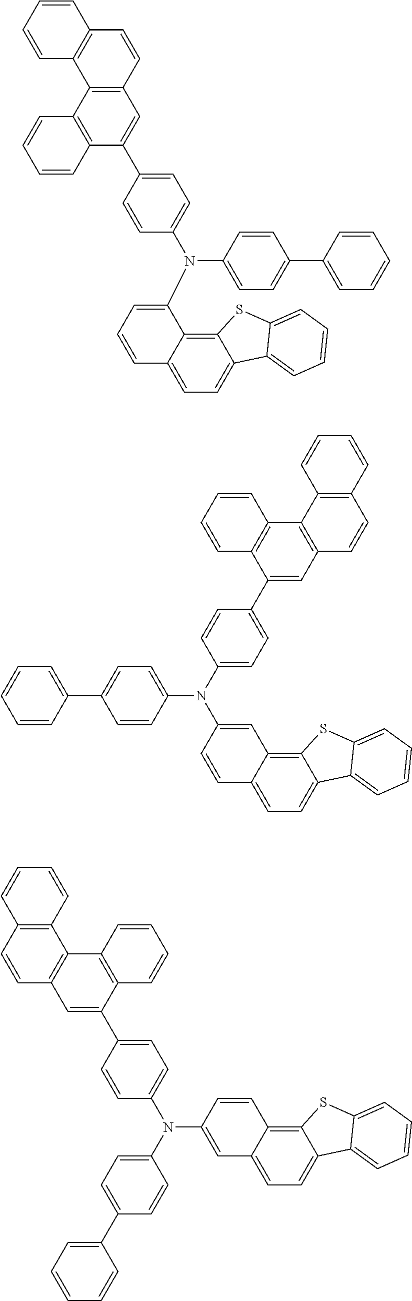 [SMILES: c1ccc(-c2ccc(N(c3ccc(-c4cc5ccc6ccccc6c5c5ccccc45)cc3)c3cccc4ccc5c6ccccc6sc5c34)cc2)cc1 ; c1ccc(-c2ccc(N(c3ccc(-c4cc5ccc6ccccc6c5c5ccccc45)cc3)c3ccc4ccc5c6ccccc6sc5c4c3)cc2)cc1 ; c1ccc(-c2ccc(N(c3ccc(-c4cc5ccc6ccccc6c5c5ccccc45)cc3)c3ccc4c(ccc5c6ccccc6sc45)c3)cc2)cc1]

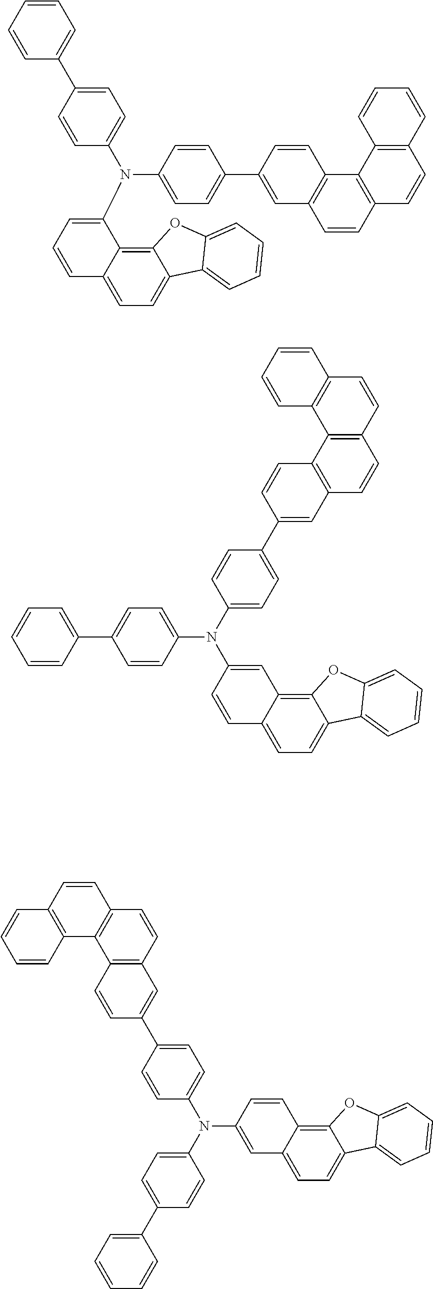 [SMILES: c1ccc(-c2ccc(N(c3ccc(-c4ccc5c(ccc6ccc7ccccc7c65)c4)cc3)c3cccc4ccc5c6ccccc6oc5c34)cc2)cc1 ; c1ccc(-c2ccc(N(c3ccc(-c4ccc5c(ccc6ccc7ccccc7c65)c4)cc3)c3ccc4ccc5c6ccccc6oc5c4c3)cc2)cc1 ; c1ccc(-c2ccc(N(c3ccc(-c4ccc5c(ccc6ccc7ccccc7c65)c4)cc3)c3ccc4c(ccc5c6ccccc6oc45)c3)cc2)cc1]

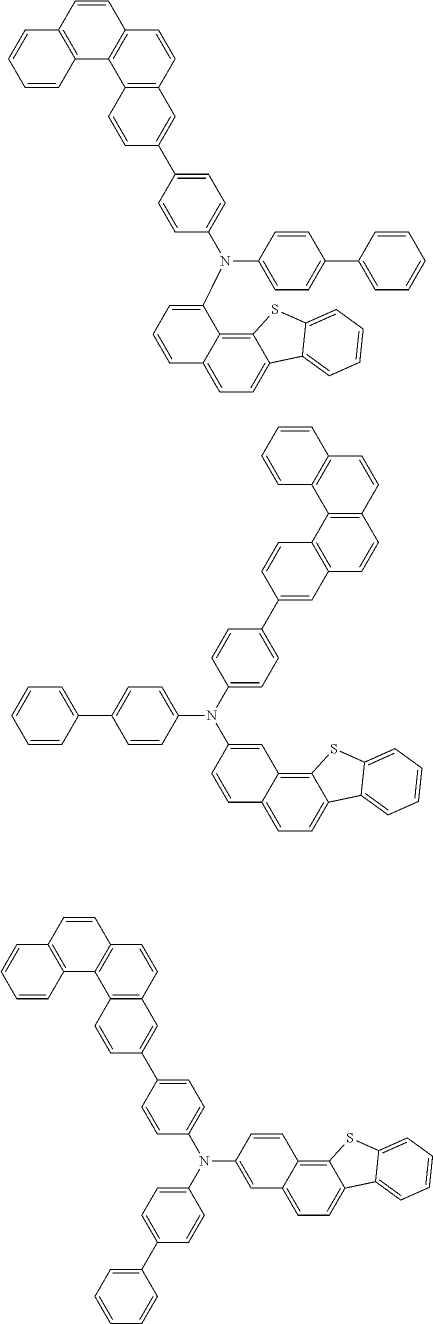

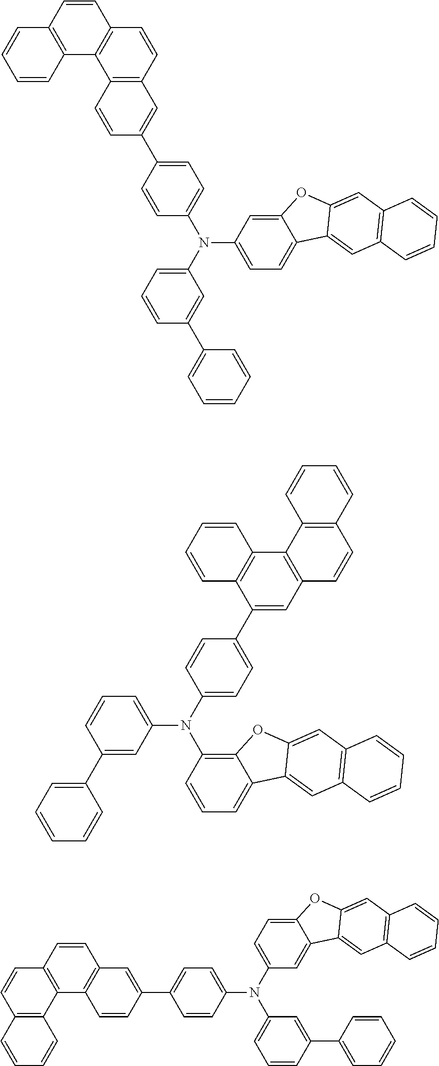 [SMILES: c1ccc(-c2cccc(N(c3ccc(-c4ccc5c(ccc6ccc7ccccc7c65)c4)cc3)c3ccc4c(c3)oc3cc5ccccc5cc34)c2)cc1 ; c1ccc(-c2cccc(N(c3ccc(-c4cc5ccc6ccccc6c5c5ccccc45)cc3)c3cccc4c3oc3cc5ccccc5cc34)c2)cc1 ; c1ccc(-c2cccc(N(c3ccc(-c4ccc5c(ccc6ccc7ccccc7c65)c4)cc3)c3ccc4oc5cc6ccccc6cc5c4c3)c2)cc1]

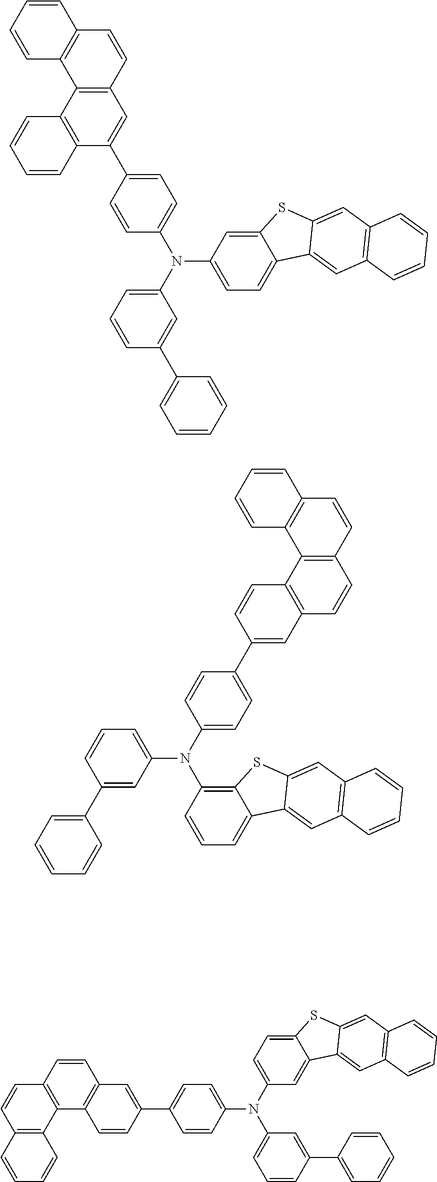 [SMILES: c1ccc(-c2cccc(N(c3ccc(-c4cc5ccc6ccccc6c5c5ccccc45)cc3)c3ccc4c(c3)sc3cc5ccccc5cc34)c2)cc1 ; c1ccc(-c2cccc(N(c3ccc(-c4ccc5c(ccc6ccc7ccccc7c65)c4)cc3)c3cccc4c3sc3cc5ccccc5cc34)c2)cc1 ; c1ccc(-c2cccc(N(c3ccc(-c4ccc5c(ccc6ccc7ccccc7c65)c4)cc3)c3ccc4sc5cc6ccccc6cc5c4c3)c2)cc1]

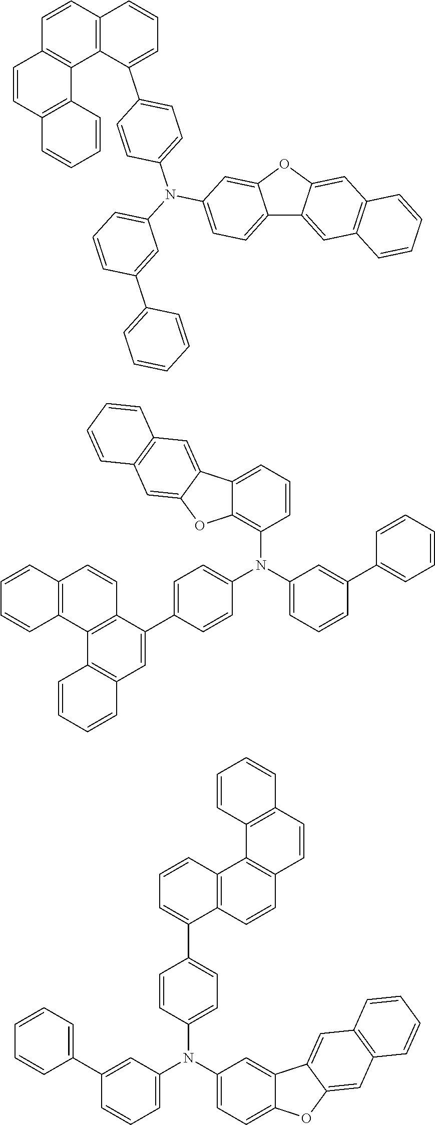

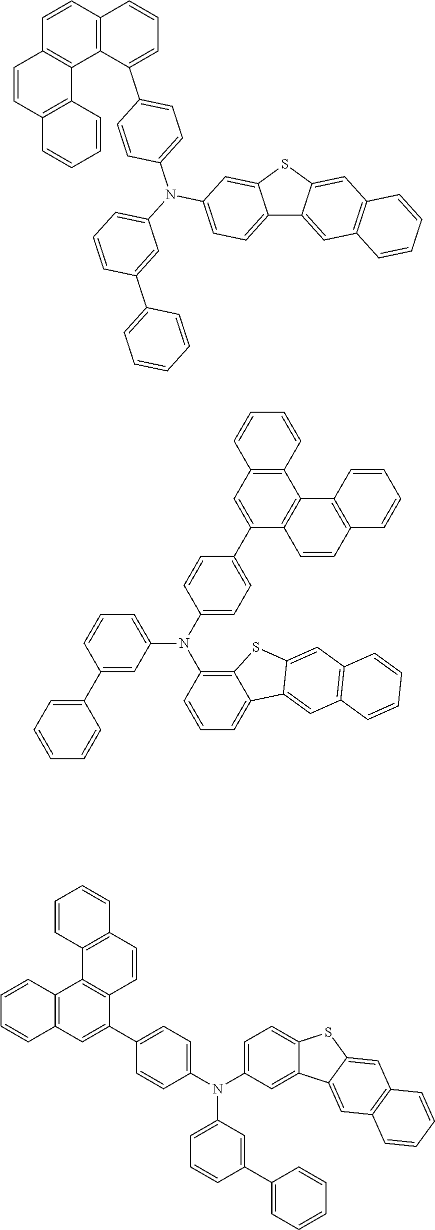 [SMILES: c1ccc(-c2cccc(N(c3ccc(-c4cccc5ccc6ccc7ccccc7c6c45)cc3)c3ccc4c(c3)sc3cc5ccccc5cc34)c2)cc1 ; c1ccc(-c2cccc(N(c3ccc(-c4cc5ccccc5c5c4ccc4ccccc45)cc3)c3cccc4c3sc3cc5ccccc5cc34)c2)cc1 ; c1ccc(-c2cccc(N(c3ccc(-c4cc5ccccc5c5c4ccc4ccccc45)cc3)c3ccc4sc5cc6ccccc6cc5c4c3)c2)cc1]

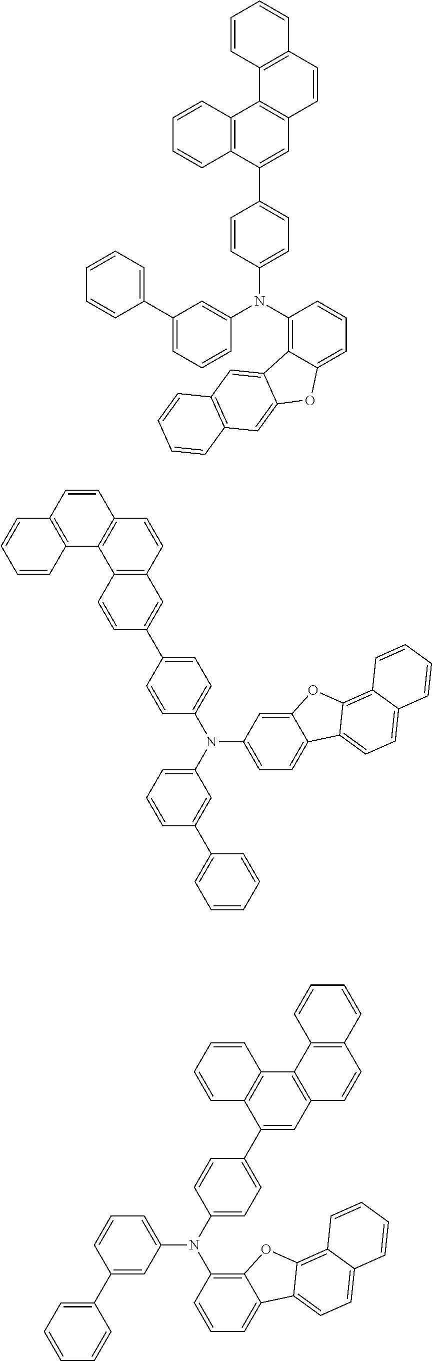 [SMILES: c1ccc(-c2cccc(N(c3ccc(-c4cc5ccc6ccccc6c5c5ccccc45)cc3)c3cccc4oc5cc6ccccc6cc5c34)c2)cc1 ; c1ccc(-c2cccc(N(c3ccc(-c4ccc5c(ccc6ccc7ccccc7c65)c4)cc3)c3ccc4c(c3)oc3c5ccccc5ccc43)c2)cc1 ; c1ccc(-c2cccc(N(c3ccc(-c4cc5ccc6ccccc6c5c5ccccc45)cc3)c3cccc4c3oc3c5ccccc5ccc43)c2)cc1]

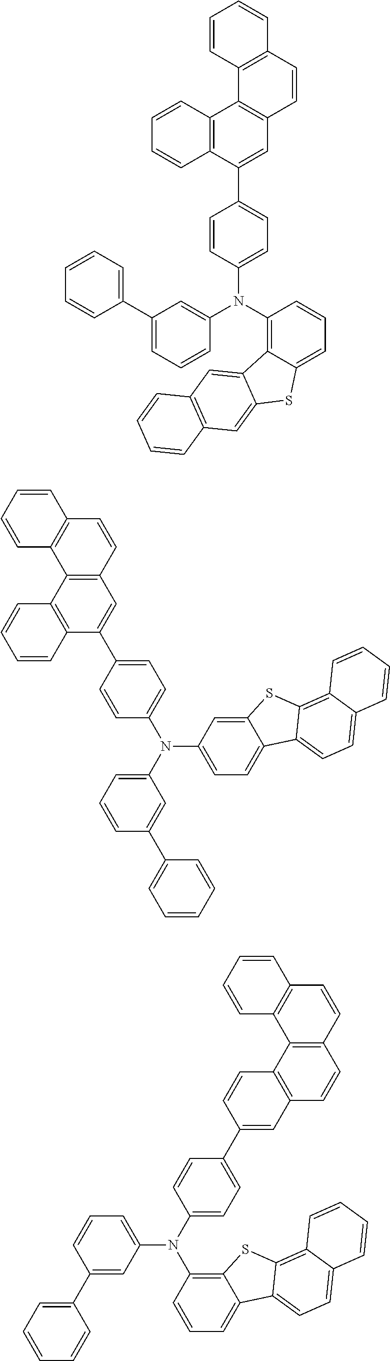 [SMILES: c1ccc(-c2cccc(N(c3ccc(-c4cc5ccc6ccccc6c5c5ccccc45)cc3)c3cccc4sc5cc6ccccc6cc5c34)c2)cc1 ; c1ccc(-c2cccc(N(c3ccc(-c4cc5ccc6ccccc6c5c5ccccc45)cc3)c3ccc4c(c3)sc3c5ccccc5ccc43)c2)cc1 ; c1ccc(-c2cccc(N(c3ccc(-c4ccc5c(ccc6ccc7ccccc7c65)c4)cc3)c3cccc4c3sc3c5ccccc5ccc43)c2)cc1]

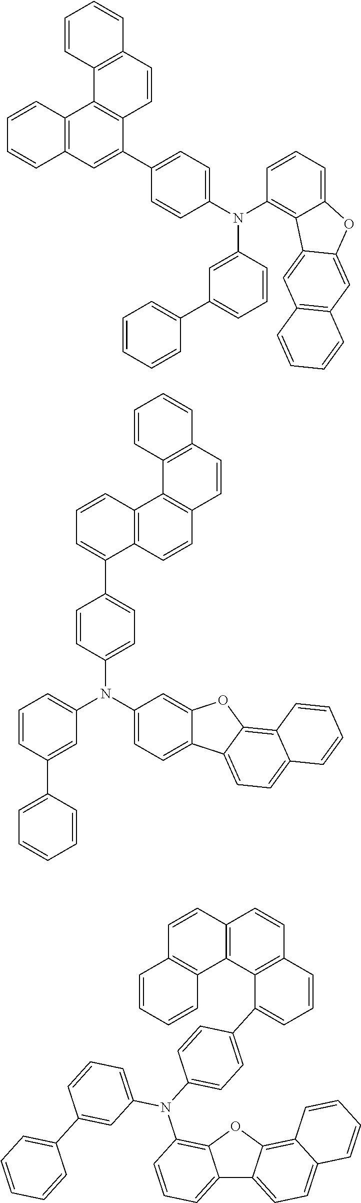

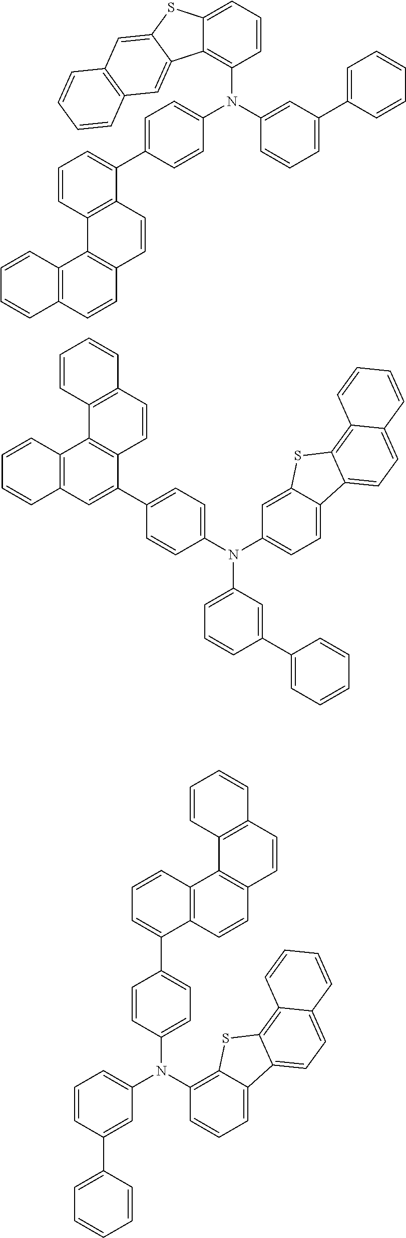

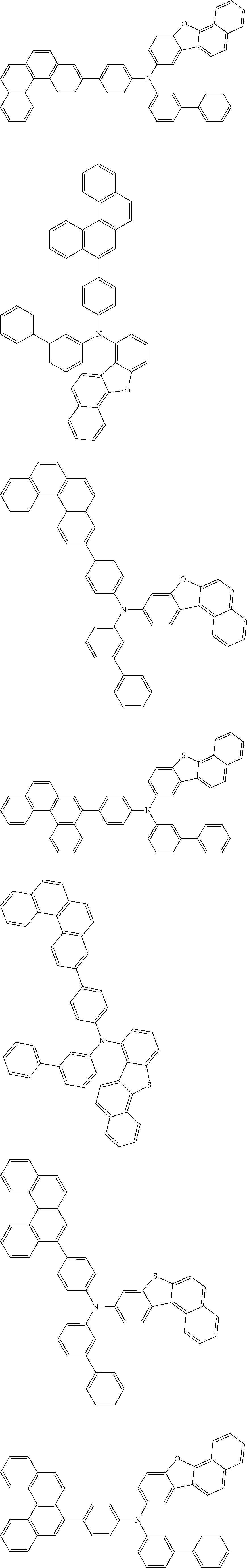

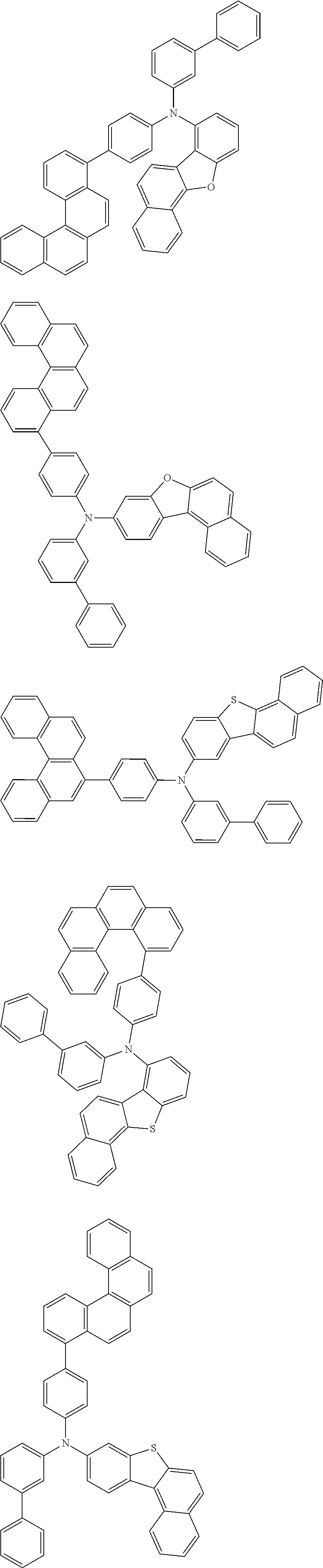

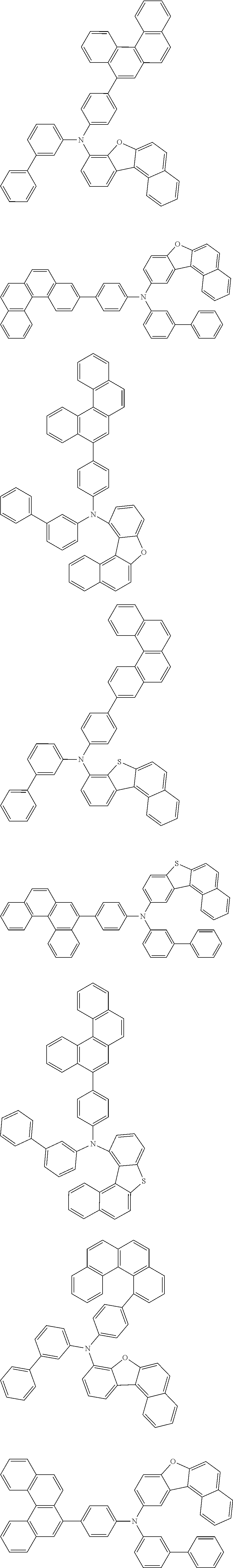

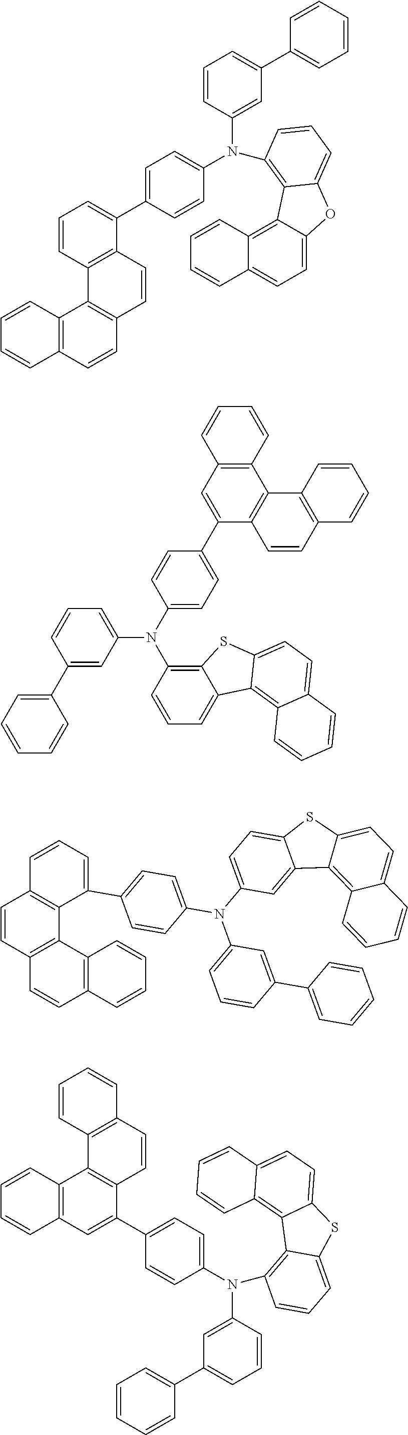 [SMILES: c1ccc(-c2cccc(N(c3ccc(-c4cccc5c4ccc4ccc6ccccc6c45)cc3)c3cccc4oc5ccc6ccccc6c5c34)c2)cc1 ; c1ccc(-c2cccc(N(c3ccc(-c4cc5ccccc5c5c4ccc4ccccc45)cc3)c3cccc4c3sc3ccc5ccccc5c34)c2)cc1 ; c1ccc(-c2cccc(N(c3ccc(-c4cccc5ccc6ccc7ccccc7c6c45)cc3)c3ccc4sc5ccc6ccccc6c5c4c3)c2)cc1 ; c1ccc(-c2cccc(N(c3ccc(-c4cc5ccccc5c5c4ccc4ccccc45)cc3)c3cccc4sc5ccc6ccccc6c5c34)c2)cc1]

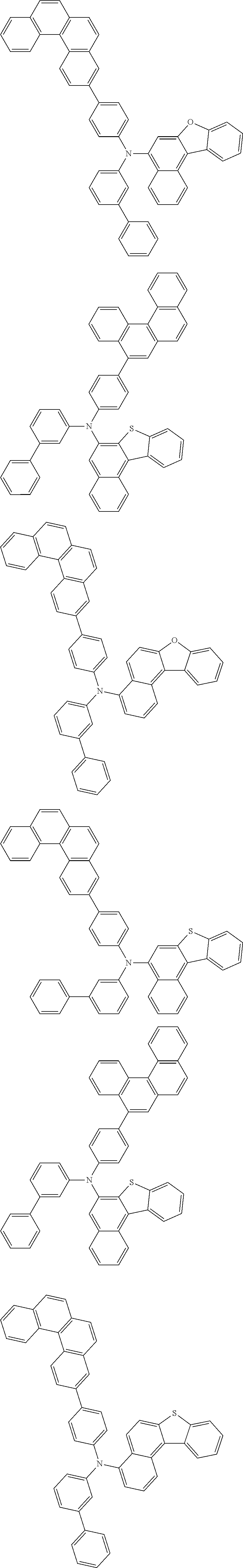

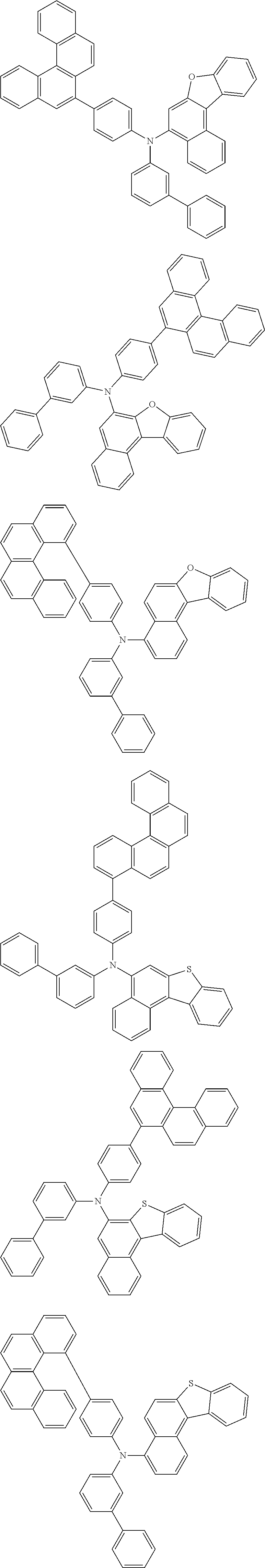

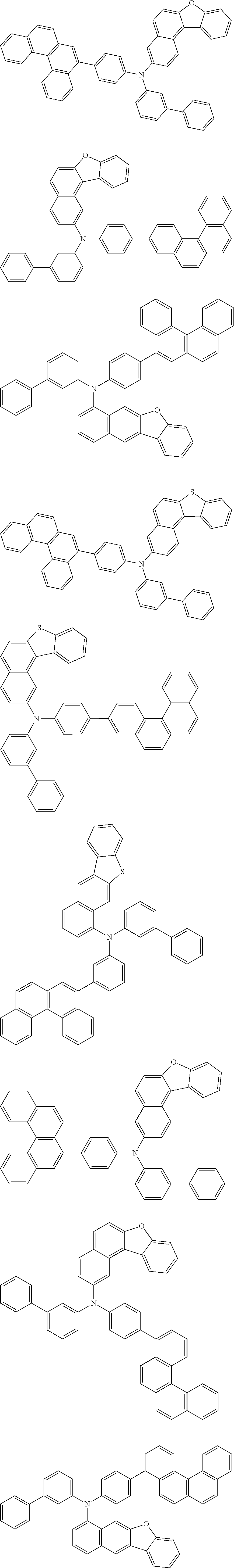

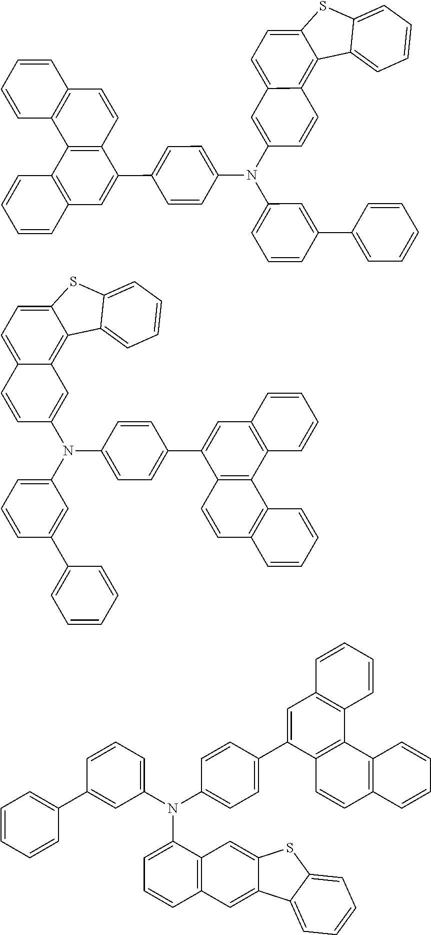 [SMILES: c1ccc(-c2cccc(N(c3ccc(-c4cc5ccccc5c5c4ccc4ccccc45)cc3)c3ccc4c(ccc5sc6ccccc6c54)c3)c2)cc1 ; c1ccc(-c2cccc(N(c3ccc(-c4cc5ccccc5c5c4ccc4ccccc45)cc3)c3ccc4ccc5sc6ccccc6c5c4c3)c2)cc1 ; c1ccc(-c2cccc(N(c3ccc(-c4cc5ccccc5c5c4ccc4ccccc45)cc3)c3cccc4cc5c(cc34)sc3ccccc35)c2)cc1]

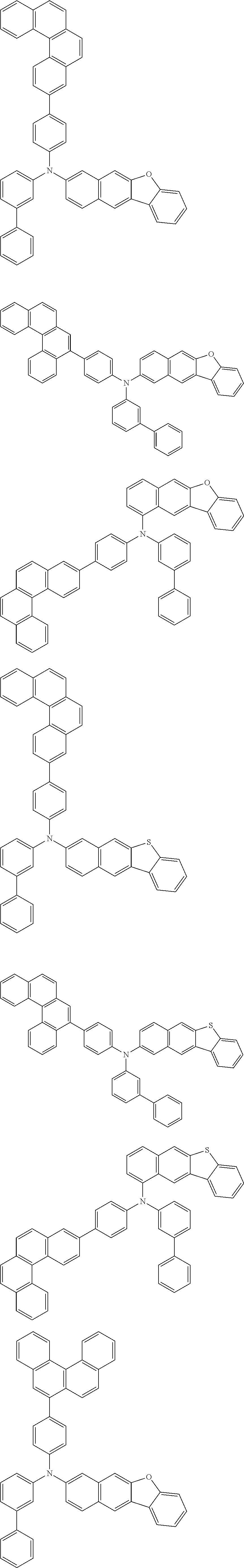

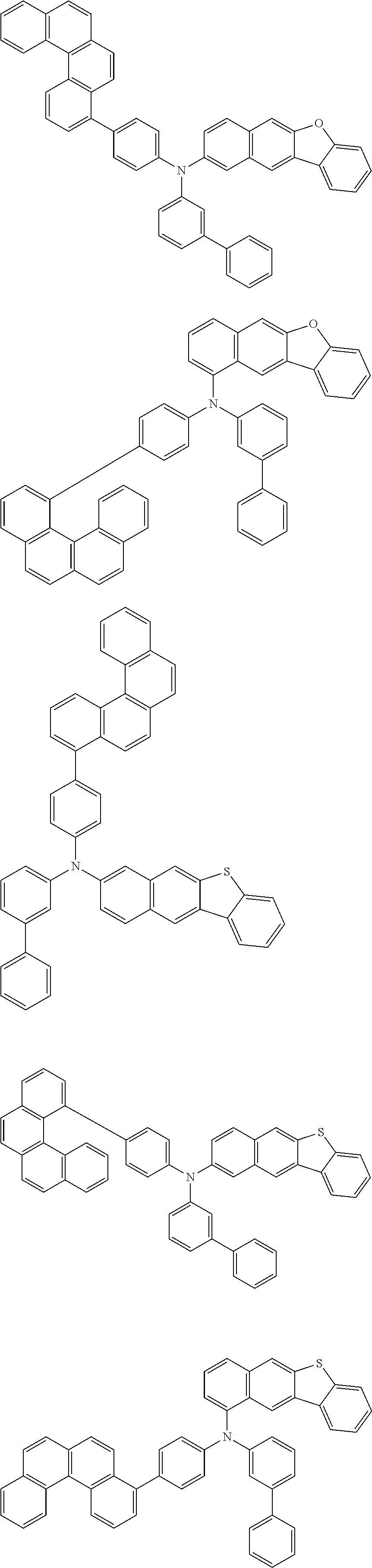

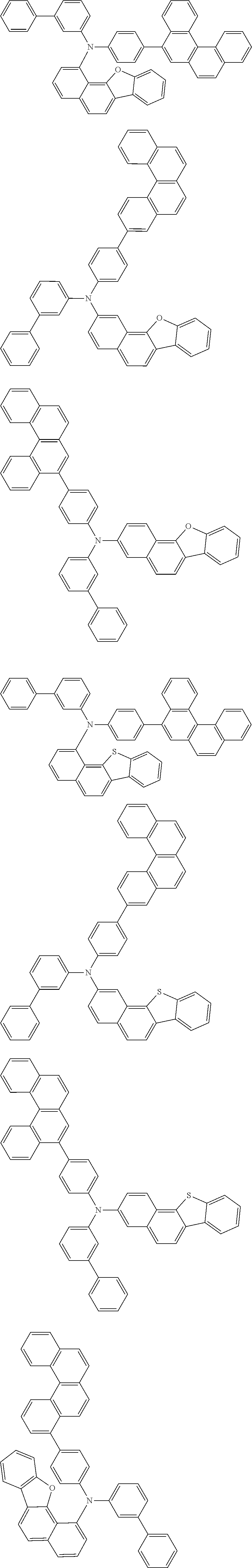

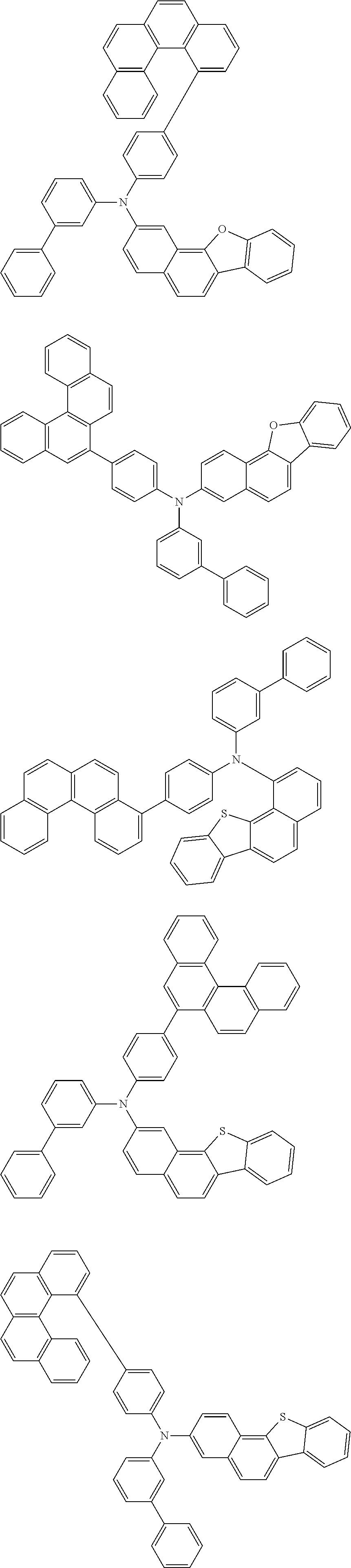

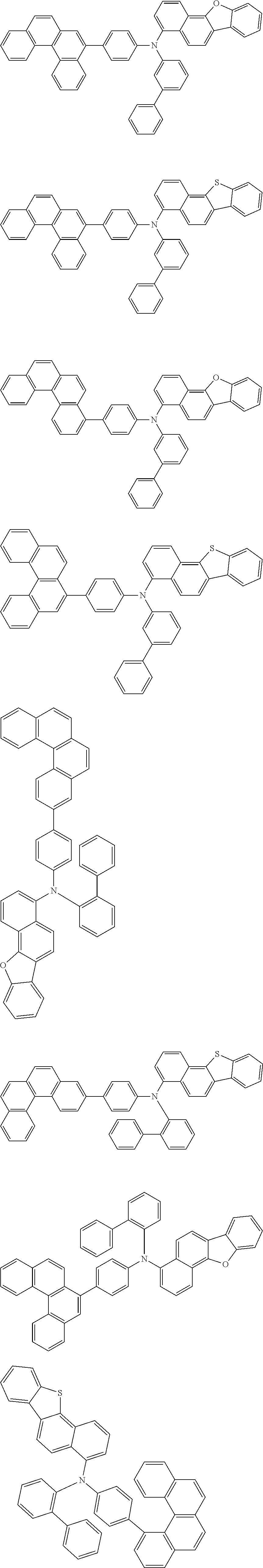

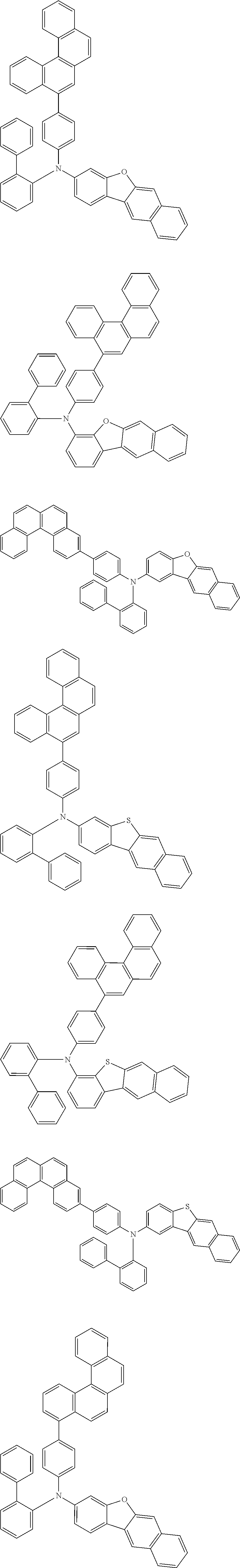

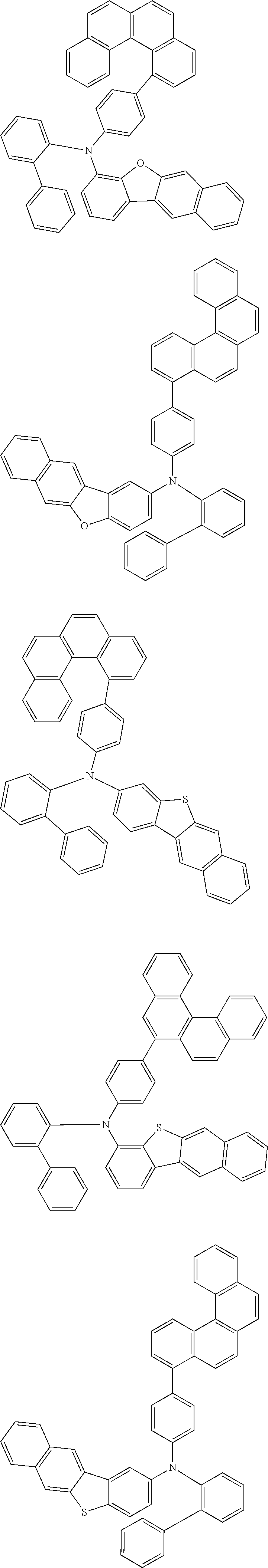

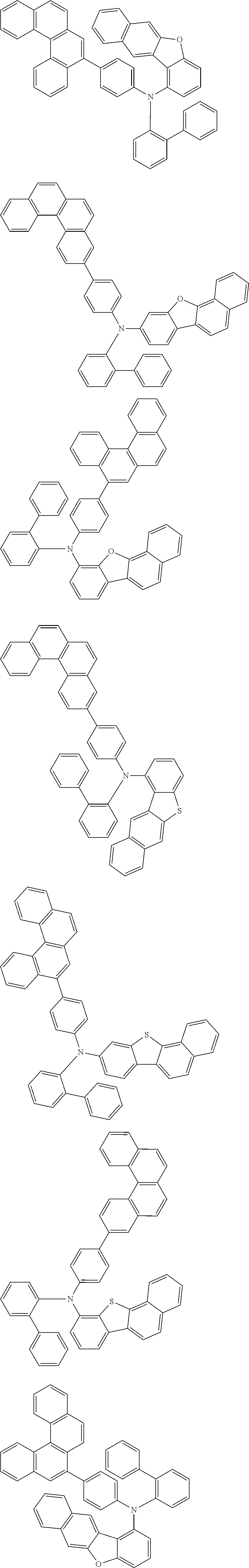

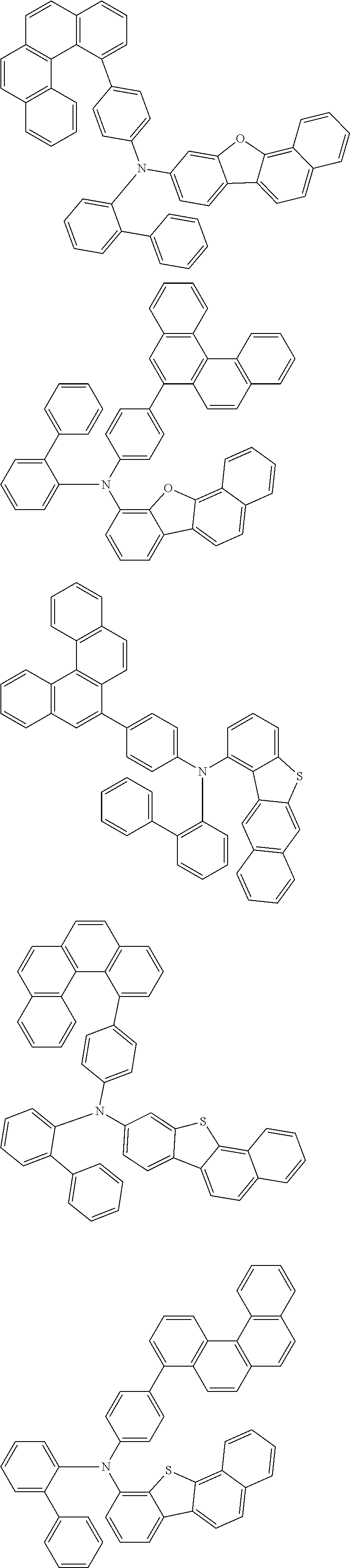

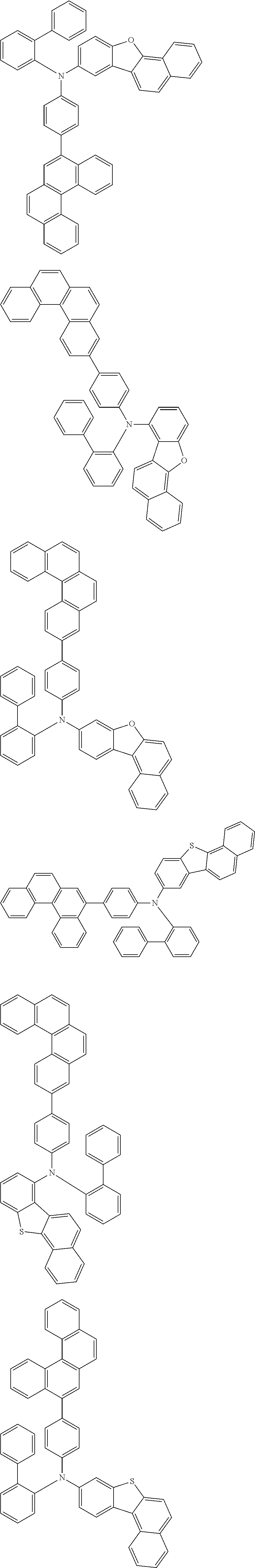

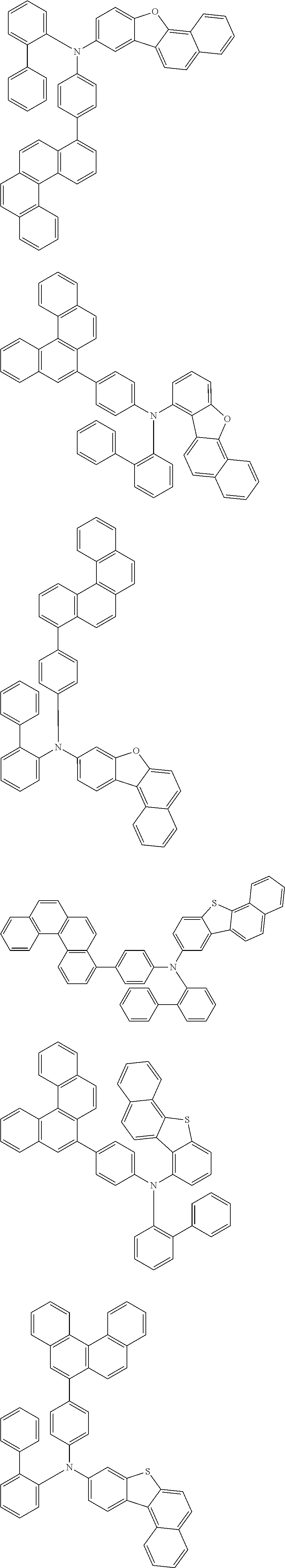

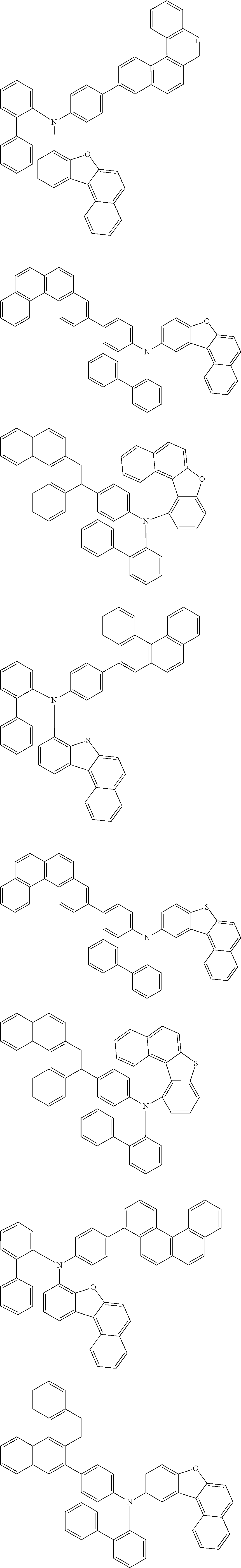

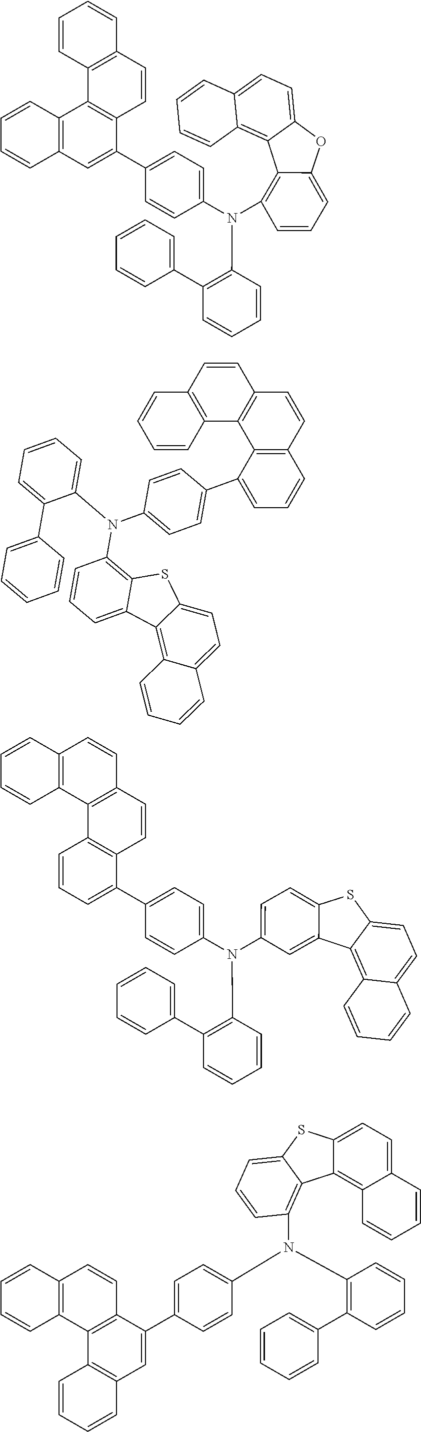

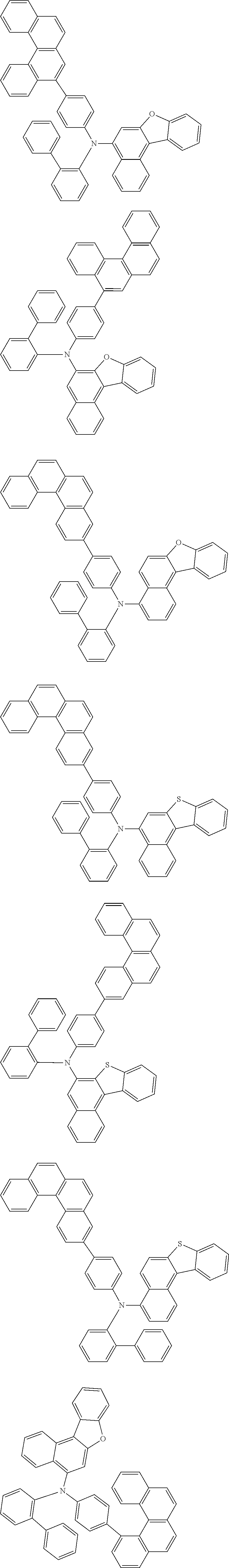

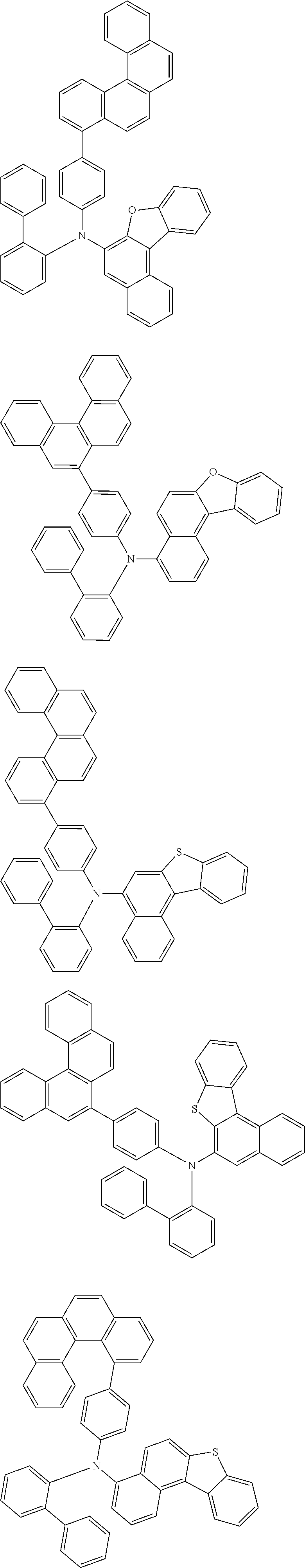

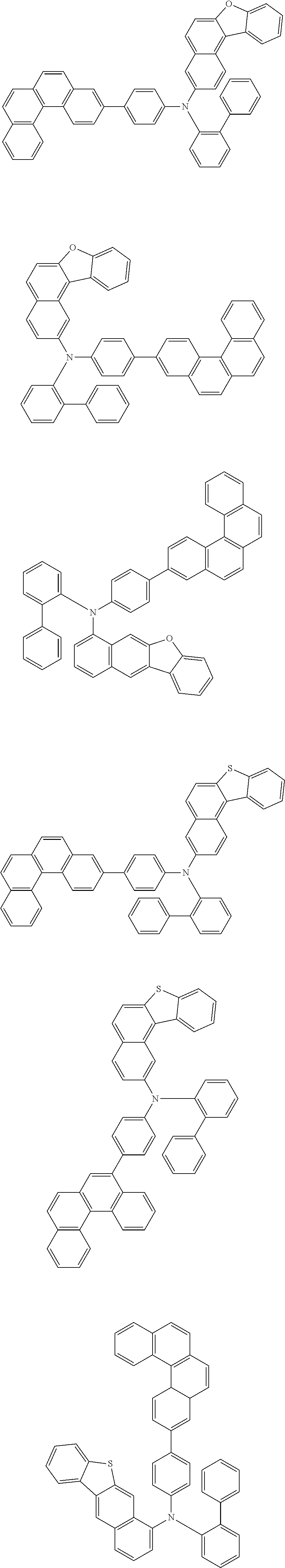

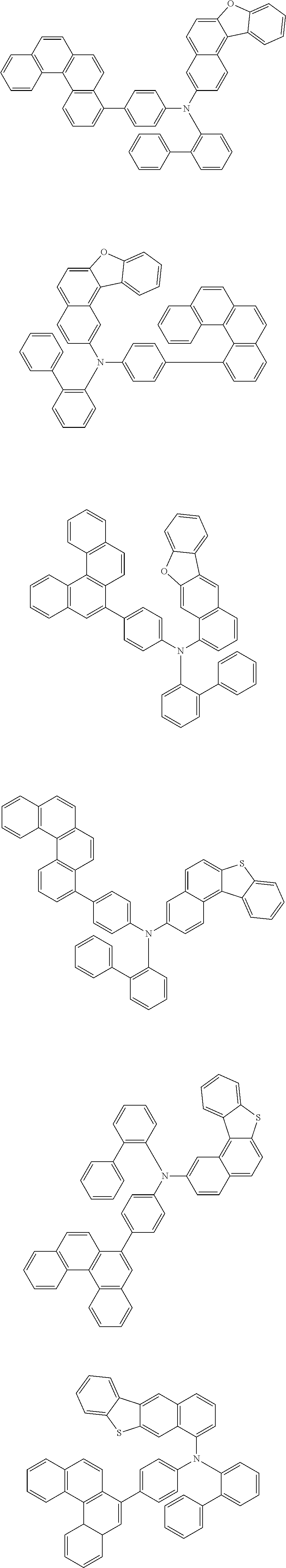

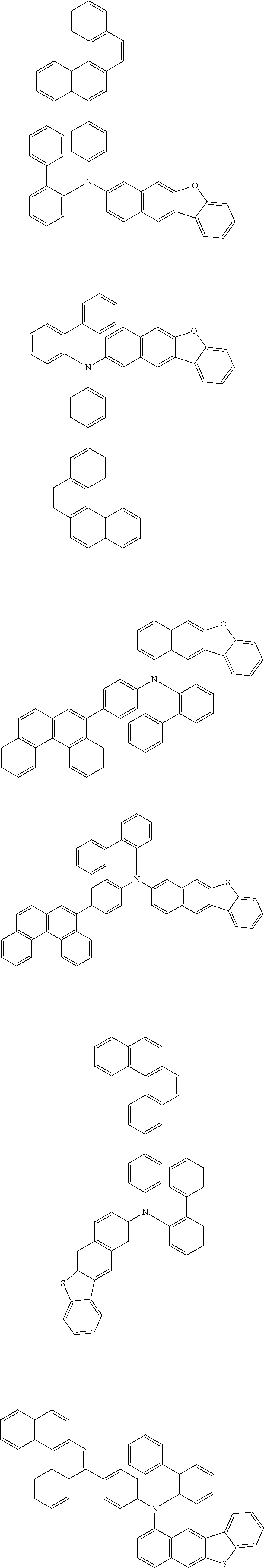

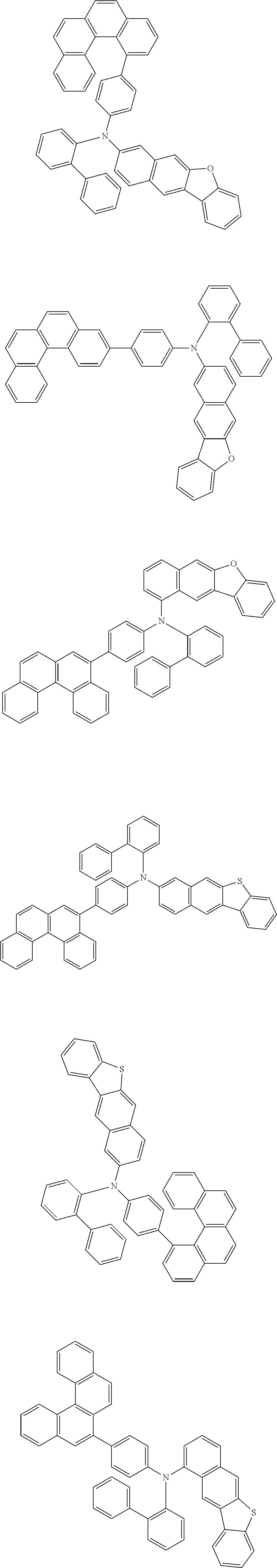

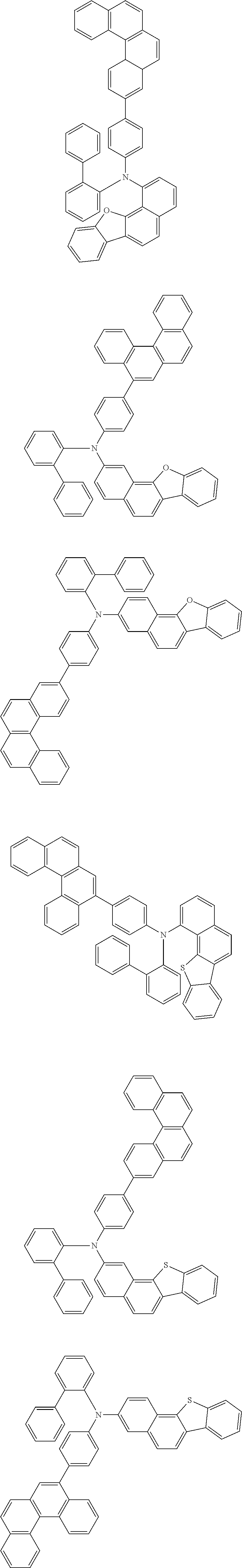

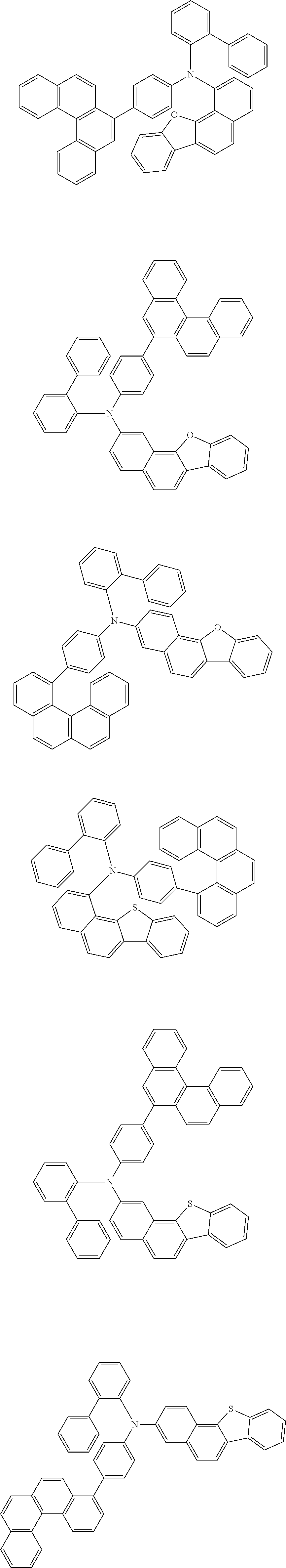

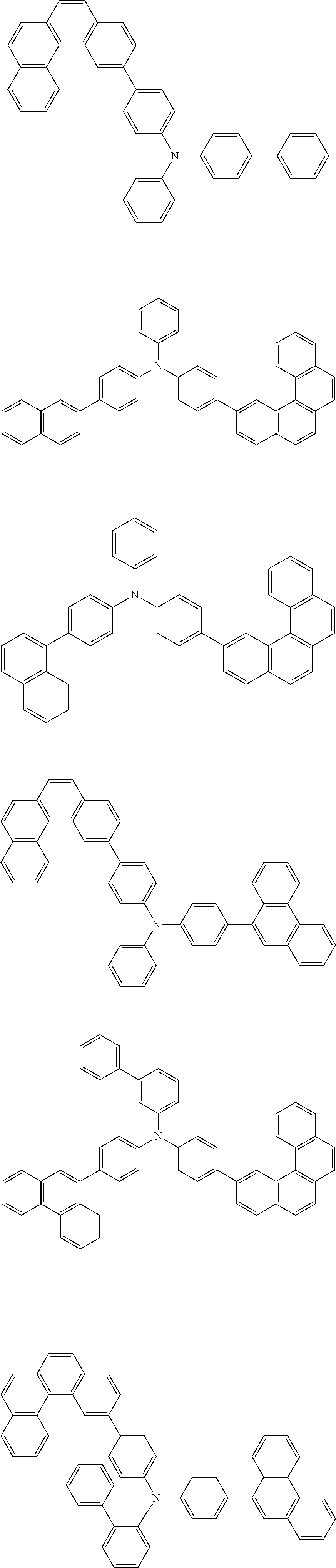 [SMILES: c1ccc(-c2ccc(N(c3ccccc3)c3ccc(-c4ccc5ccc6ccc7ccccc7c6c5c4)cc3)cc2)cc1 ; c1ccc(N(c2ccc(-c3ccc4ccccc4c3)cc2)c2ccc(-c3ccc4ccc5ccc6ccccc6c5c4c3)cc2)cc1 ; c1ccc(N(c2ccc(-c3ccc4ccc5ccc6ccccc6c5c4c3)cc2)c2ccc(-c3cccc4ccccc34)cc2)cc1 ; c1ccc(N(c2ccc(-c3ccc4ccc5ccc6ccccc6c5c4c3)cc2)c2ccc(-c3cc4ccccc4c4ccccc34)cc2)cc1 ; c1ccc(-c2cccc(N(c3ccc(-c4ccc5ccc6ccc7ccccc7c6c5c4)cc3)c3ccc(-c4cc5ccccc5c5ccccc45)cc3)c2)cc1 ; c1ccc(-c2ccccc2N(c2ccc(-c3ccc4ccc5ccc6ccccc6c5c4c3)cc2)c2ccc(-c3cc4ccccc4c4ccccc34)cc2)cc1]

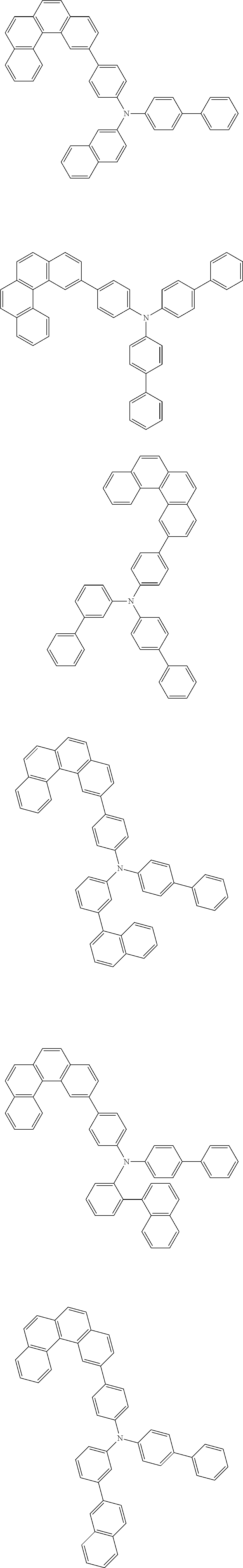

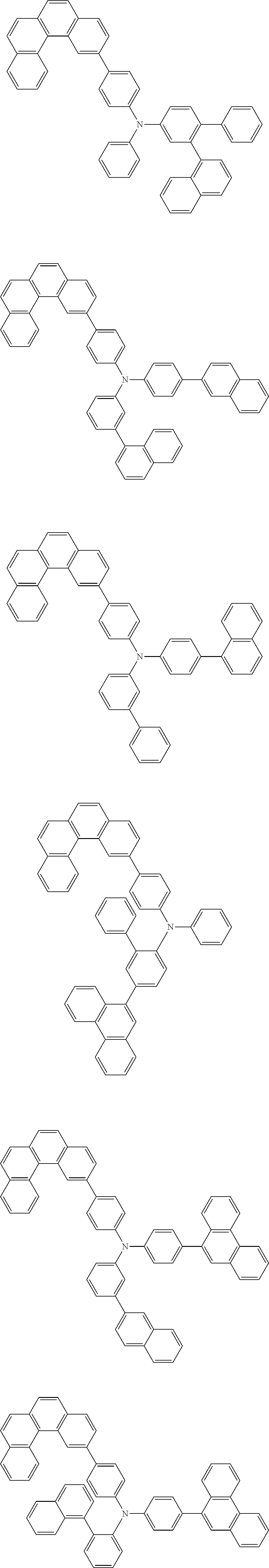

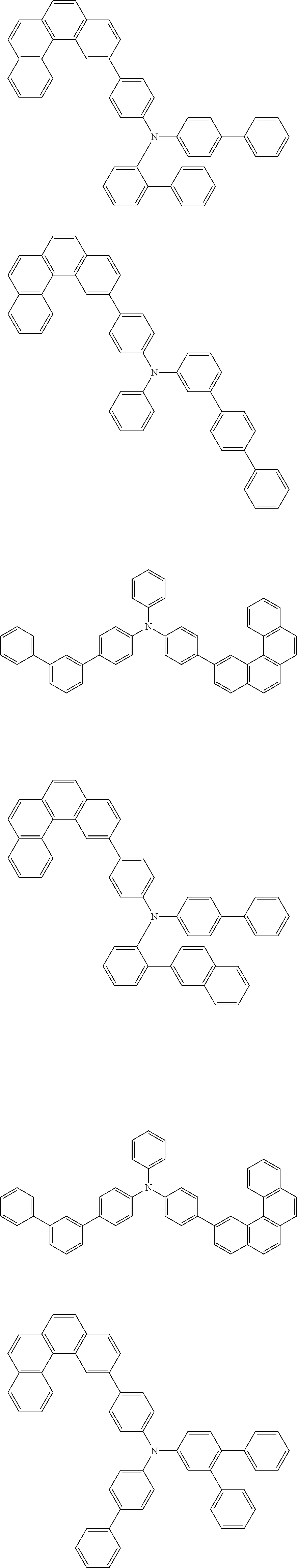

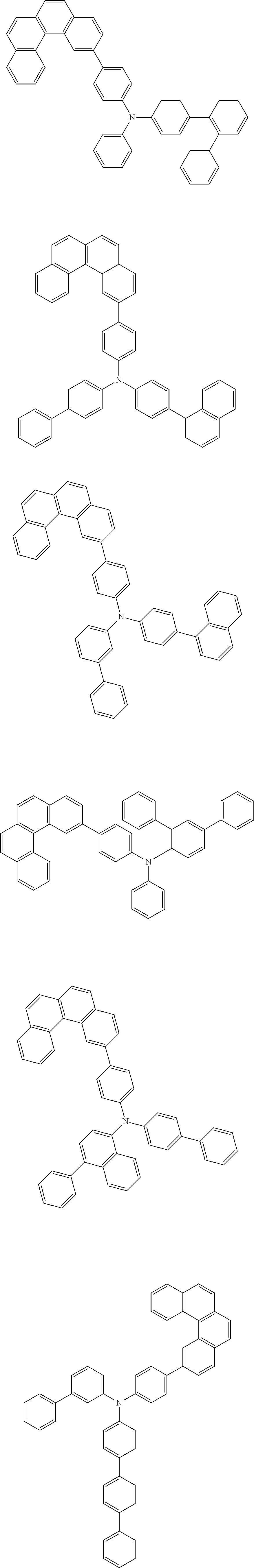

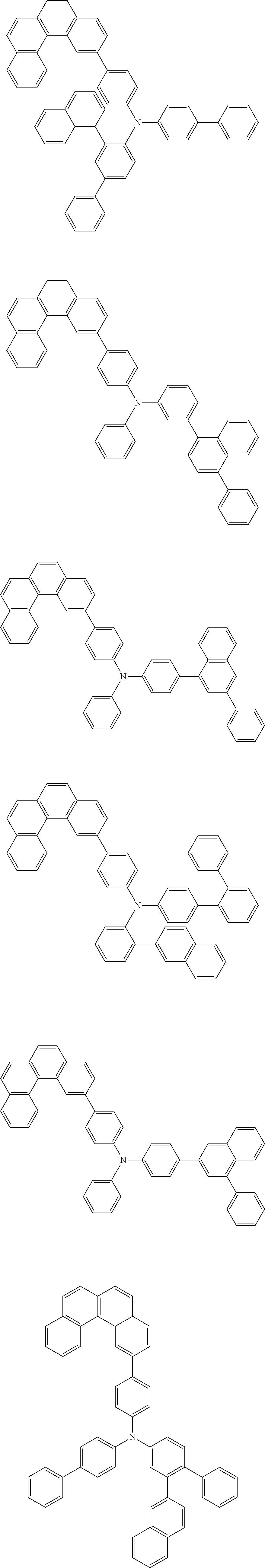

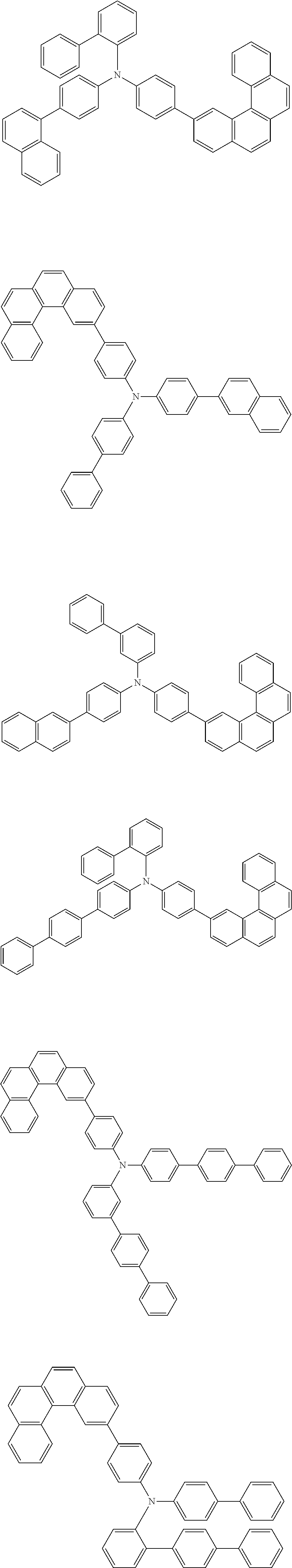

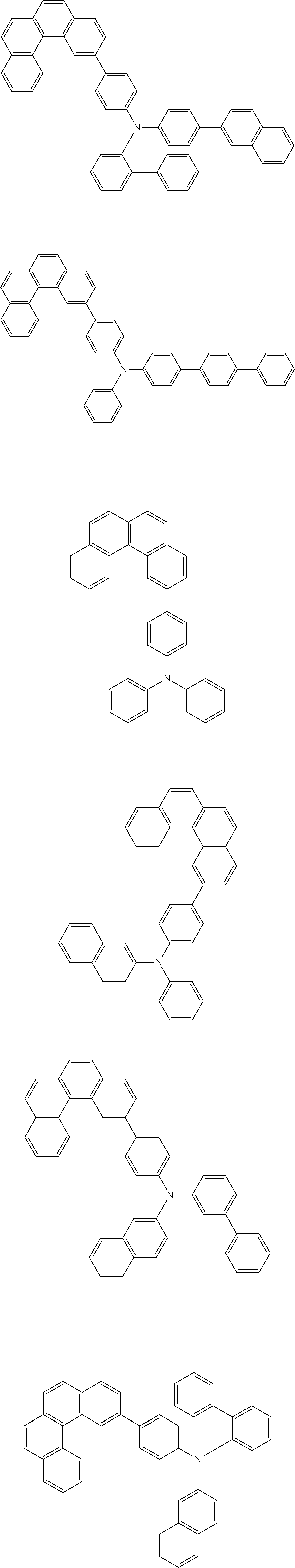 [SMILES: c1ccc(-c2ccccc2N(c2ccc(-c3ccc4ccccc4c3)cc2)c2ccc(-c3ccc4ccc5ccc6ccccc6c5c4c3)cc2)cc1 ; c1ccc(-c2ccc(-c3ccc(N(c4ccccc4)c4ccc(-c5ccc6ccc7ccc8ccccc8c7c6c5)cc4)cc3)cc2)cc1 ; c1ccc(N(c2ccccc2)c2ccc(-c3ccc4ccc5ccc6ccccc6c5c4c3)cc2)cc1 ; c1ccc(N(c2ccc(-c3ccc4ccc5ccc6ccccc6c5c4c3)cc2)c2ccc3ccccc3c2)cc1 ; c1ccc(-c2cccc(N(c3ccc(-c4ccc5ccc6ccc7ccccc7c6c5c4)cc3)c3ccc4ccccc4c3)c2)cc1 ; c1ccc(-c2ccccc2N(c2ccc(-c3ccc4ccc5ccc6ccccc6c5c4c3)cc2)c2ccc3ccccc3c2)cc1]

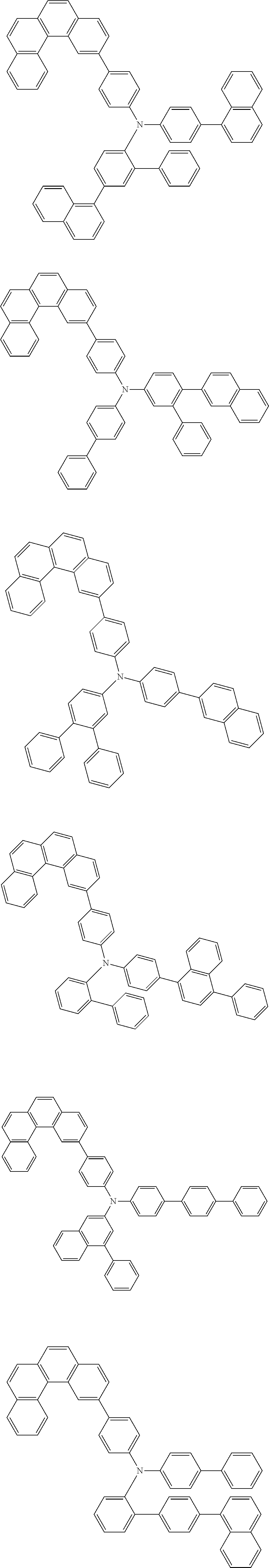

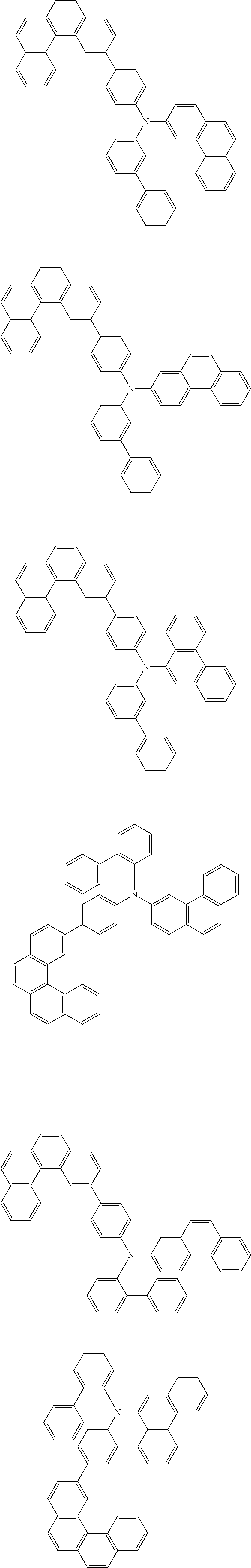

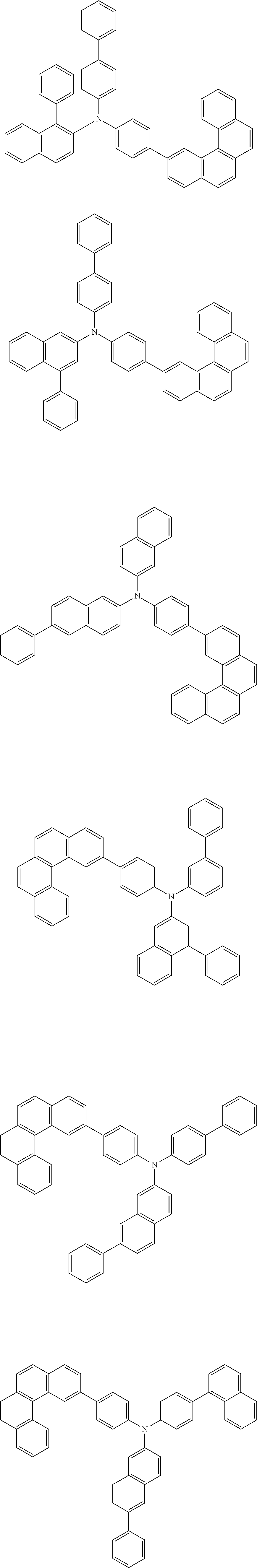

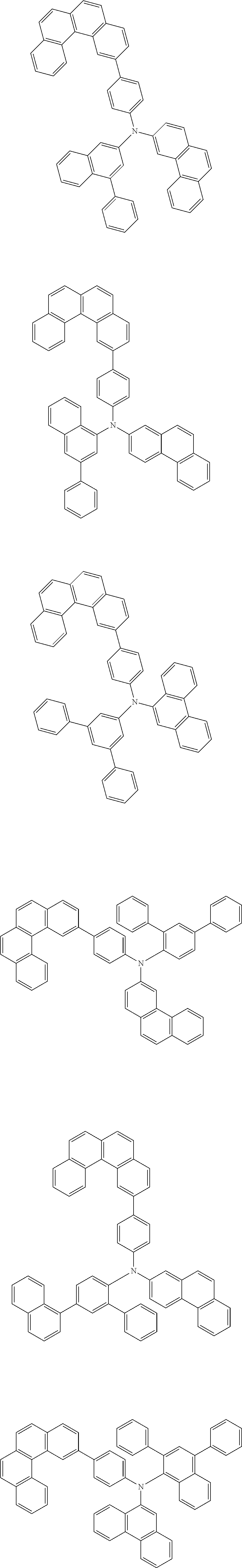

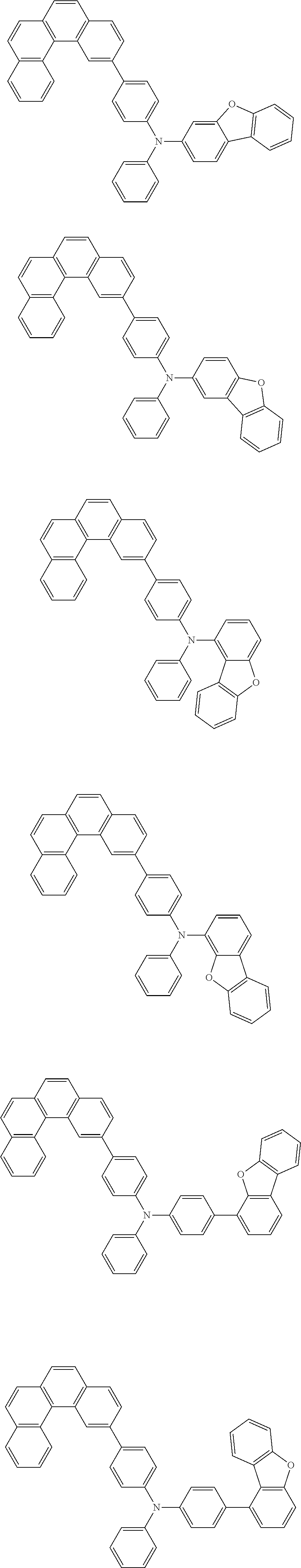 [SMILES: c1ccc(N(c2ccc(-c3ccc4ccc5ccc6ccccc6c5c4c3)cc2)c2ccc3c(c2)oc2ccccc23)cc1 ; c1ccc(N(c2ccc(-c3ccc4ccc5ccc6ccccc6c5c4c3)cc2)c2ccc3oc4ccccc4c3c2)cc1 ; c1ccc(N(c2ccc(-c3ccc4ccc5ccc6ccccc6c5c4c3)cc2)c2cccc3oc4ccccc4c23)cc1 ; c1ccc(N(c2ccc(-c3ccc4ccc5ccc6ccccc6c5c4c3)cc2)c2cccc3c2oc2ccccc23)cc1 ; c1ccc(N(c2ccc(-c3ccc4ccc5ccc6ccccc6c5c4c3)cc2)c2ccc(-c3cccc4c3oc3ccccc34)cc2)cc1 ; c1ccc(N(c2ccc(-c3ccc4ccc5ccc6ccccc6c5c4c3)cc2)c2ccc(-c3cccc4oc5ccccc5c34)cc2)cc1]

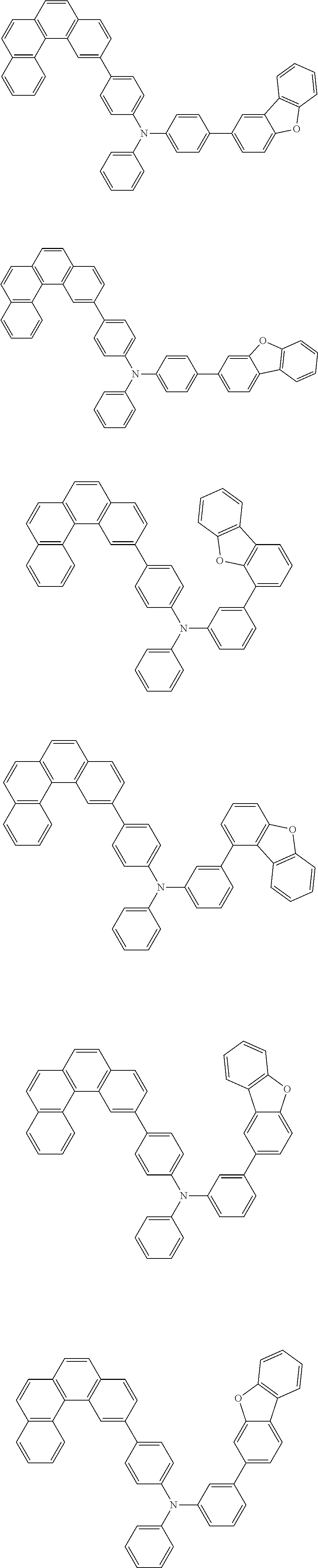 [SMILES: c1ccc(N(c2ccc(-c3ccc4oc5ccccc5c4c3)cc2)c2ccc(-c3ccc4ccc5ccc6ccccc6c5c4c3)cc2)cc1 ; c1ccc(N(c2ccc(-c3ccc4c(c3)oc3ccccc34)cc2)c2ccc(-c3ccc4ccc5ccc6ccccc6c5c4c3)cc2)cc1 ; c1ccc(N(c2ccc(-c3ccc4ccc5ccc6ccccc6c5c4c3)cc2)c2cccc(-c3cccc4c3oc3ccccc34)c2)cc1 ; c1ccc(N(c2ccc(-c3ccc4ccc5ccc6ccccc6c5c4c3)cc2)c2cccc(-c3cccc4oc5ccccc5c34)c2)cc1 ; c1ccc(N(c2ccc(-c3ccc4ccc5ccc6ccccc6c5c4c3)cc2)c2cccc(-c3ccc4oc5ccccc5c4c3)c2)cc1 ; c1ccc(N(c2ccc(-c3ccc4ccc5ccc6ccccc6c5c4c3)cc2)c2cccc(-c3ccc4c(c3)oc3ccccc34)c2)cc1]

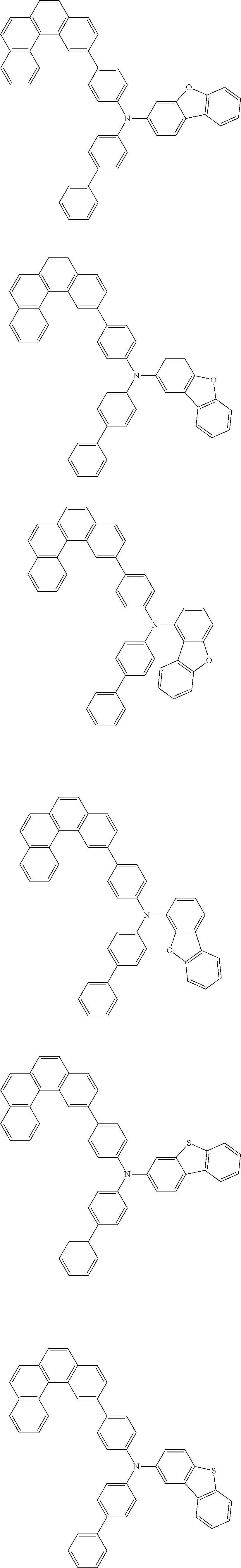 [SMILES: c1ccc(-c2ccc(N(c3ccc(-c4ccc5ccc6ccc7ccccc7c6c5c4)cc3)c3ccc4c(c3)oc3ccccc34)cc2)cc1 ; c1ccc(-c2ccc(N(c3ccc(-c4ccc5ccc6ccc7ccccc7c6c5c4)cc3)c3ccc4oc5ccccc5c4c3)cc2)cc1 ; c1ccc(-c2ccc(N(c3ccc(-c4ccc5ccc6ccc7ccccc7c6c5c4)cc3)c3cccc4oc5ccccc5c34)cc2)cc1 ; c1ccc(-c2ccc(N(c3ccc(-c4ccc5ccc6ccc7ccccc7c6c5c4)cc3)c3cccc4c3oc3ccccc34)cc2)cc1 ; c1ccc(-c2ccc(N(c3ccc(-c4ccc5ccc6ccc7ccccc7c6c5c4)cc3)c3ccc4c(c3)sc3ccccc34)cc2)cc1 ; c1ccc(-c2ccc(N(c3ccc(-c4ccc5ccc6ccc7ccccc7c6c5c4)cc3)c3ccc4sc5ccccc5c4c3)cc2)cc1]

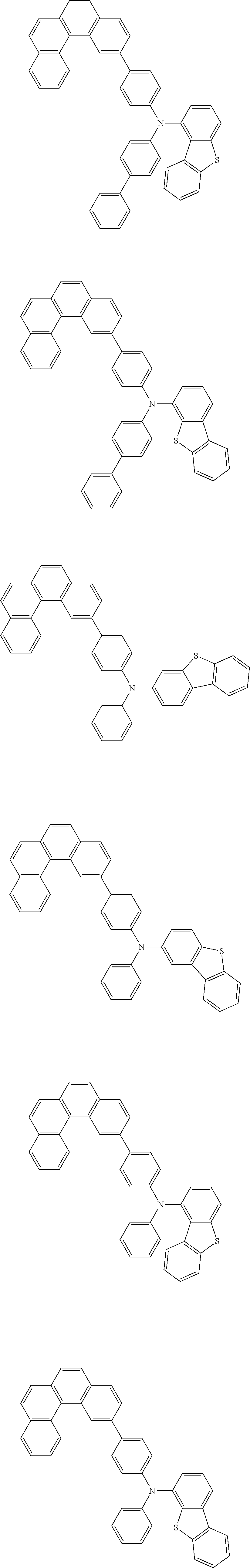 [SMILES: c1ccc(-c2ccc(N(c3ccc(-c4ccc5ccc6ccc7ccccc7c6c5c4)cc3)c3cccc4sc5ccccc5c34)cc2)cc1 ; c1ccc(-c2ccc(N(c3ccc(-c4ccc5ccc6ccc7ccccc7c6c5c4)cc3)c3cccc4c3sc3ccccc34)cc2)cc1 ; c1ccc(N(c2ccc(-c3ccc4ccc5ccc6ccccc6c5c4c3)cc2)c2ccc3c(c2)sc2ccccc23)cc1 ; c1ccc(N(c2ccc(-c3ccc4ccc5ccc6ccccc6c5c4c3)cc2)c2ccc3sc4ccccc4c3c2)cc1 ; c1ccc(N(c2ccc(-c3ccc4ccc5ccc6ccccc6c5c4c3)cc2)c2cccc3sc4ccccc4c23)cc1 ; c1ccc(N(c2ccc(-c3ccc4ccc5ccc6ccccc6c5c4c3)cc2)c2cccc3c2sc2ccccc23)cc1]

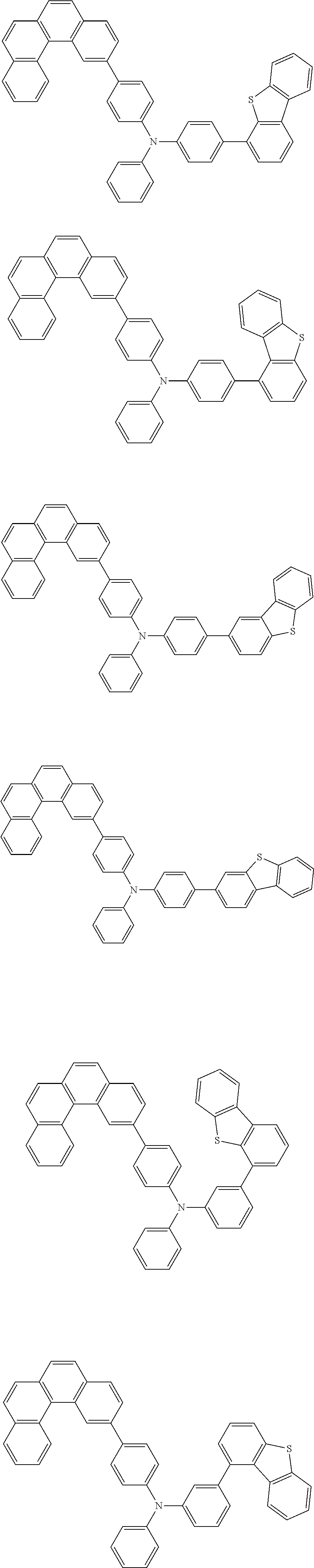 [SMILES: c1ccc(N(c2ccc(-c3ccc4ccc5ccc6ccccc6c5c4c3)cc2)c2ccc(-c3cccc4c3sc3ccccc34)cc2)cc1 ; c1ccc(N(c2ccc(-c3ccc4ccc5ccc6ccccc6c5c4c3)cc2)c2ccc(-c3cccc4sc5ccccc5c34)cc2)cc1 ; c1ccc(N(c2ccc(-c3ccc4sc5ccccc5c4c3)cc2)c2ccc(-c3ccc4ccc5ccc6ccccc6c5c4c3)cc2)cc1 ; c1ccc(N(c2ccc(-c3ccc4c(c3)sc3ccccc34)cc2)c2ccc(-c3ccc4ccc5ccc6ccccc6c5c4c3)cc2)cc1 ; c1ccc(N(c2ccc(-c3ccc4ccc5ccc6ccccc6c5c4c3)cc2)c2cccc(-c3cccc4c3sc3ccccc34)c2)cc1 ; c1ccc(N(c2ccc(-c3ccc4ccc5ccc6ccccc6c5c4c3)cc2)c2cccc(-c3cccc4sc5ccccc5c34)c2)cc1]

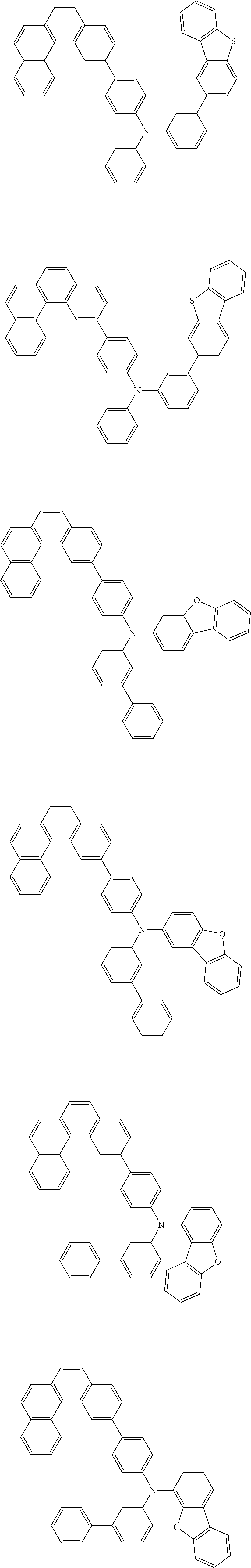 [SMILES: c1ccc(N(c2ccc(-c3ccc4ccc5ccc6ccccc6c5c4c3)cc2)c2cccc(-c3ccc4sc5ccccc5c4c3)c2)cc1 ; c1ccc(N(c2ccc(-c3ccc4ccc5ccc6ccccc6c5c4c3)cc2)c2cccc(-c3ccc4c(c3)sc3ccccc34)c2)cc1 ; c1ccc(-c2cccc(N(c3ccc(-c4ccc5ccc6ccc7ccccc7c6c5c4)cc3)c3ccc4c(c3)oc3ccccc34)c2)cc1 ; c1ccc(-c2cccc(N(c3ccc(-c4ccc5ccc6ccc7ccccc7c6c5c4)cc3)c3ccc4oc5ccccc5c4c3)c2)cc1 ; c1ccc(-c2cccc(N(c3ccc(-c4ccc5ccc6ccc7ccccc7c6c5c4)cc3)c3cccc4oc5ccccc5c34)c2)cc1 ; c1ccc(-c2cccc(N(c3ccc(-c4ccc5ccc6ccc7ccccc7c6c5c4)cc3)c3cccc4c3oc3ccccc34)c2)cc1]

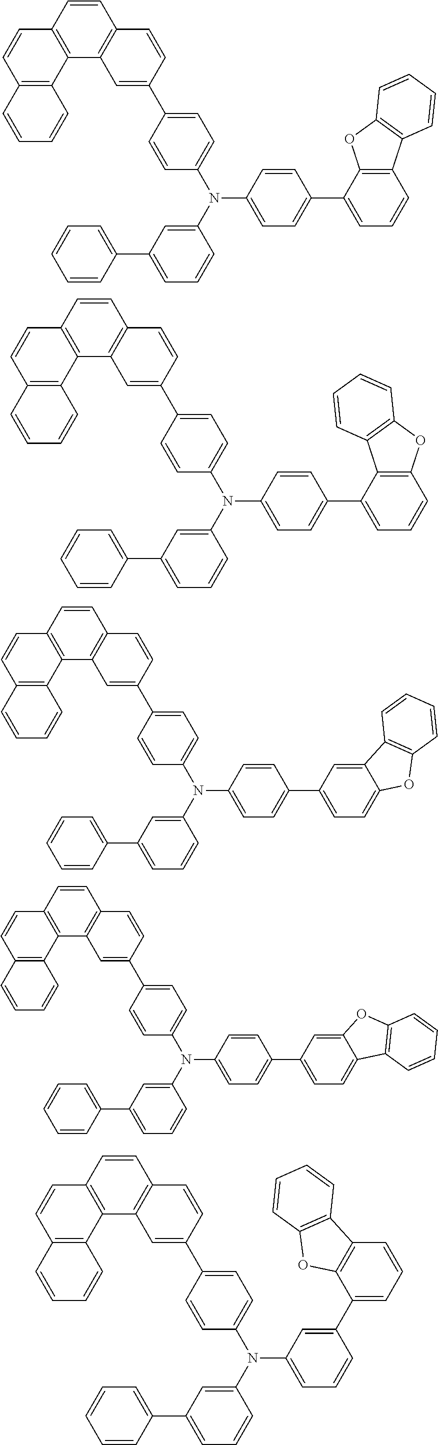

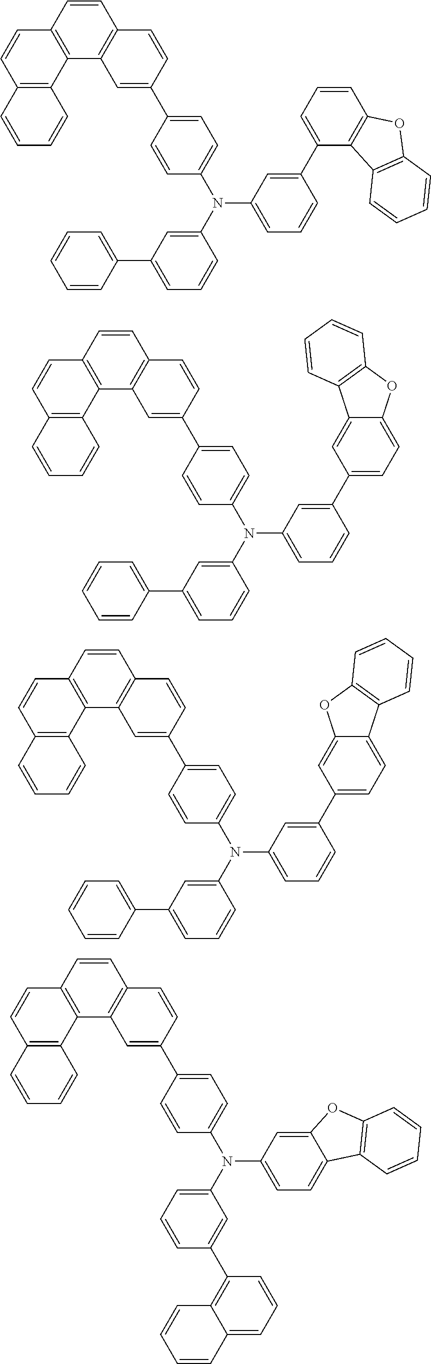

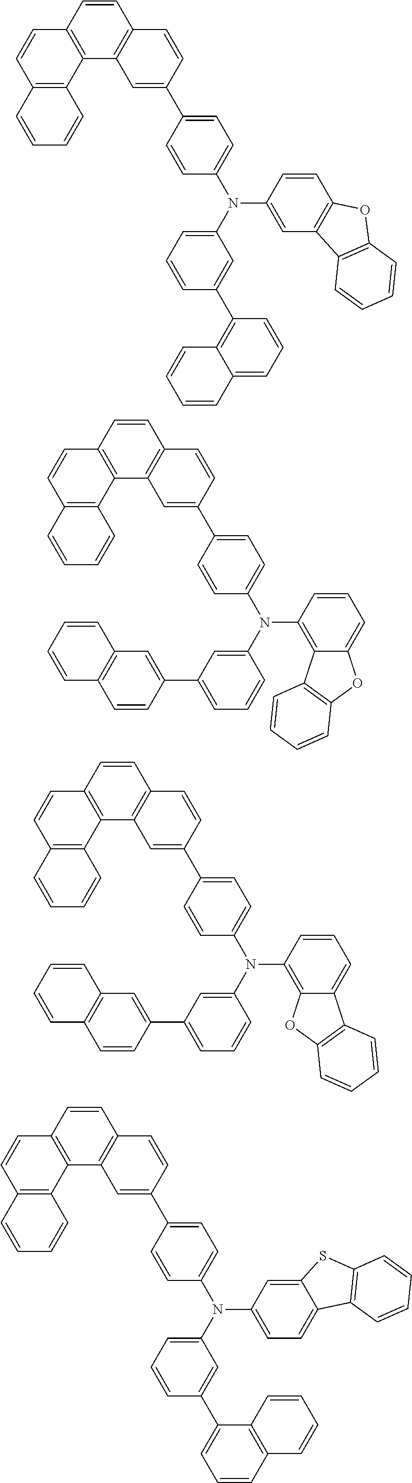

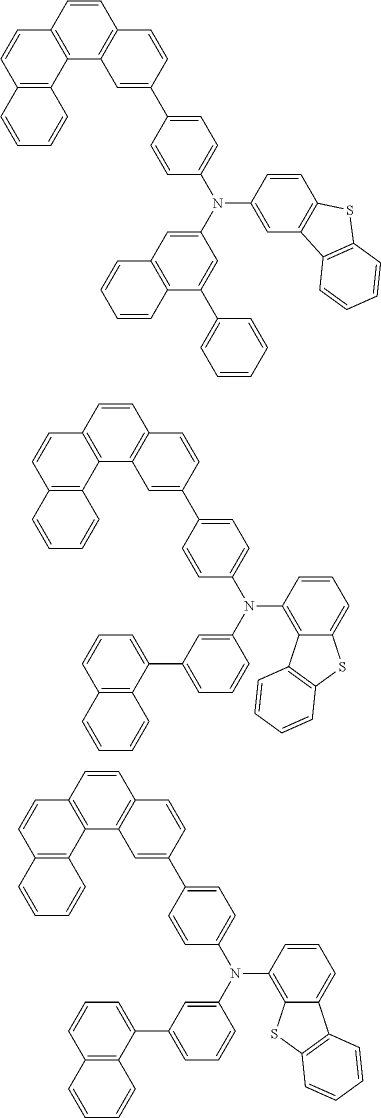

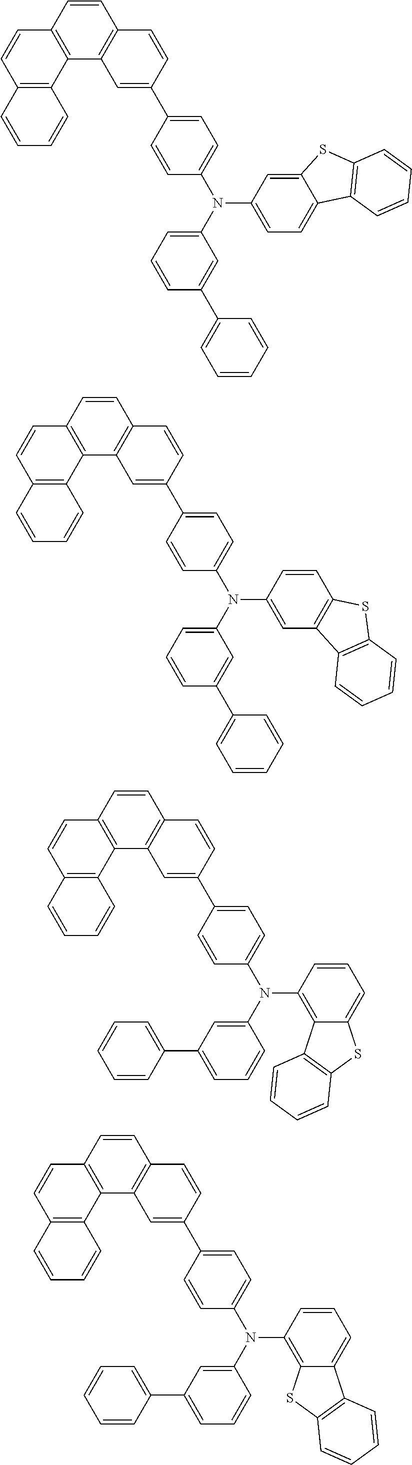 [SMILES: c1ccc(-c2cccc(N(c3ccc(-c4ccc5ccc6ccc7ccccc7c6c5c4)cc3)c3ccc4c(c3)sc3ccccc34)c2)cc1 ; c1ccc(-c2cccc(N(c3ccc(-c4ccc5ccc6ccc7ccccc7c6c5c4)cc3)c3ccc4sc5ccccc5c4c3)c2)cc1 ; c1ccc(-c2cccc(N(c3ccc(-c4ccc5ccc6ccc7ccccc7c6c5c4)cc3)c3cccc4sc5ccccc5c34)c2)cc1 ; c1ccc(-c2cccc(N(c3ccc(-c4ccc5ccc6ccc7ccccc7c6c5c4)cc3)c3cccc4c3sc3ccccc34)c2)cc1]

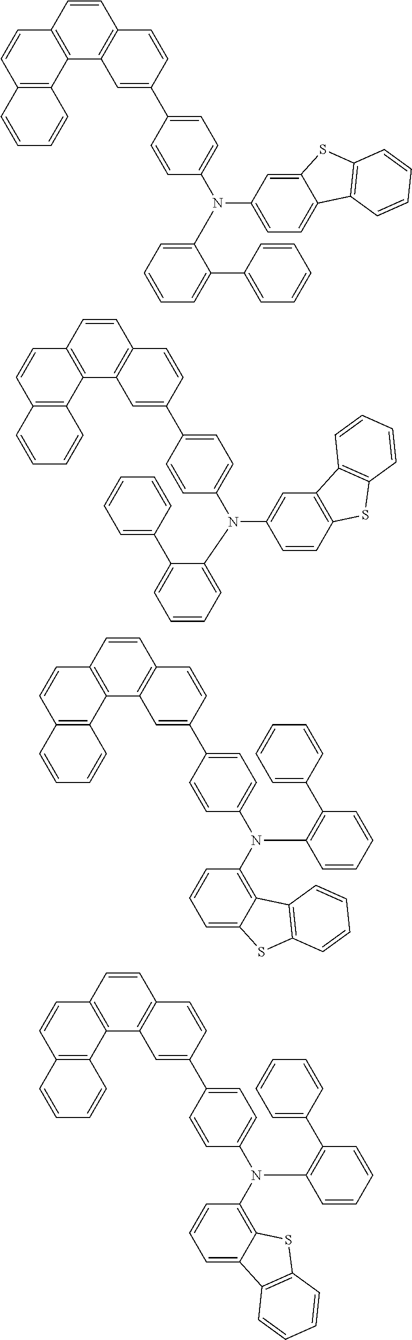

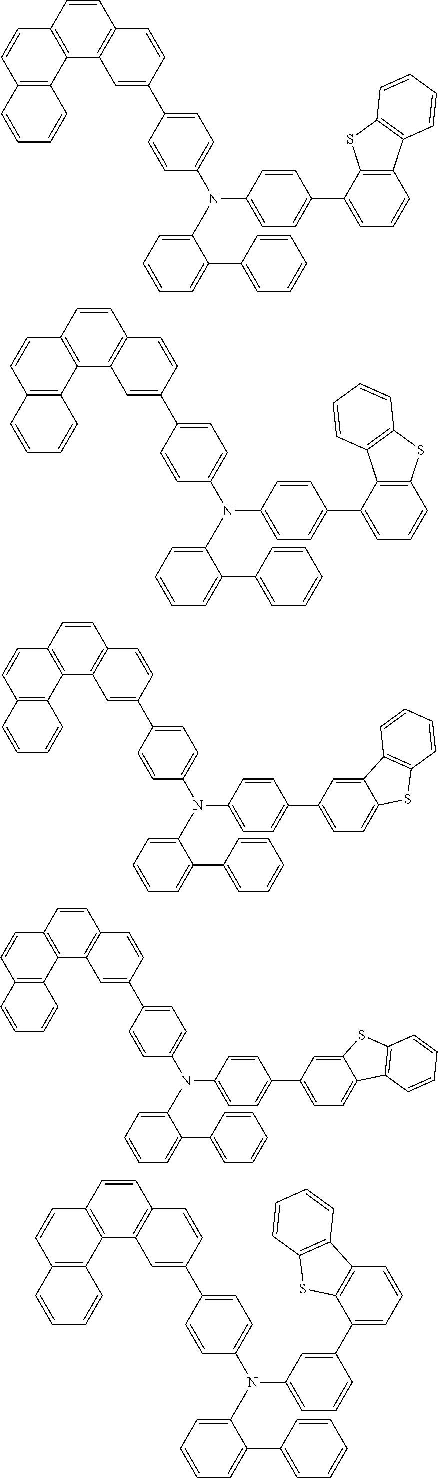

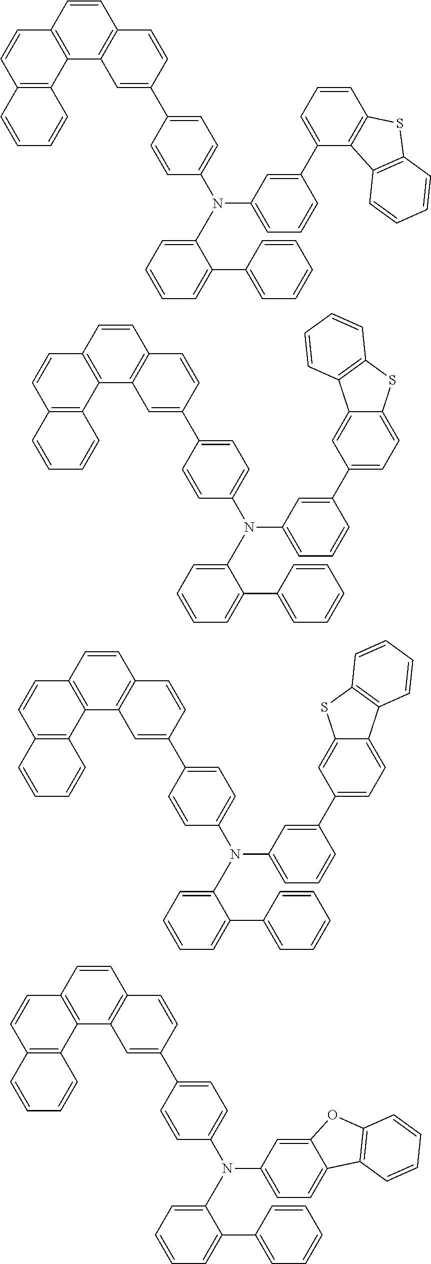

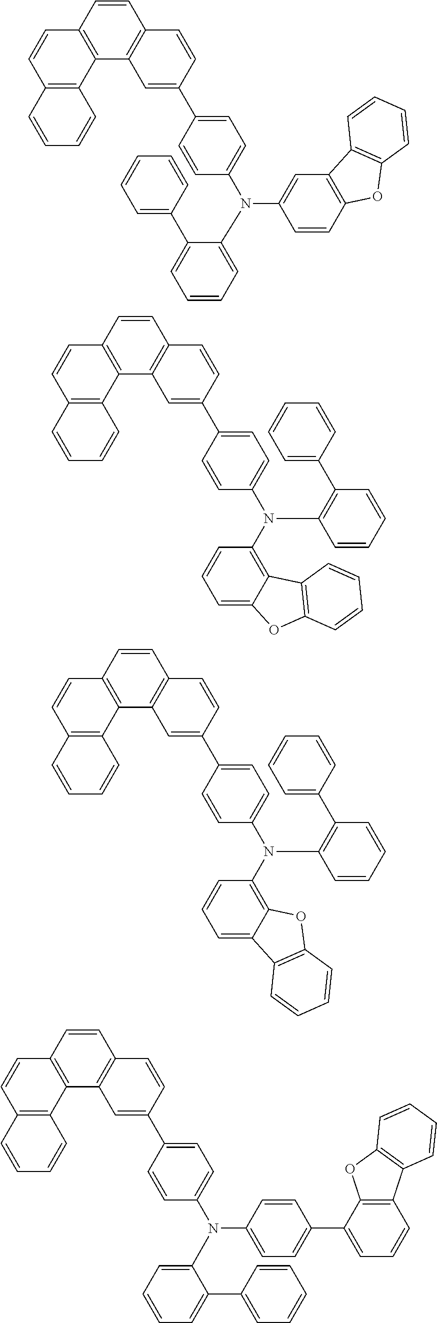

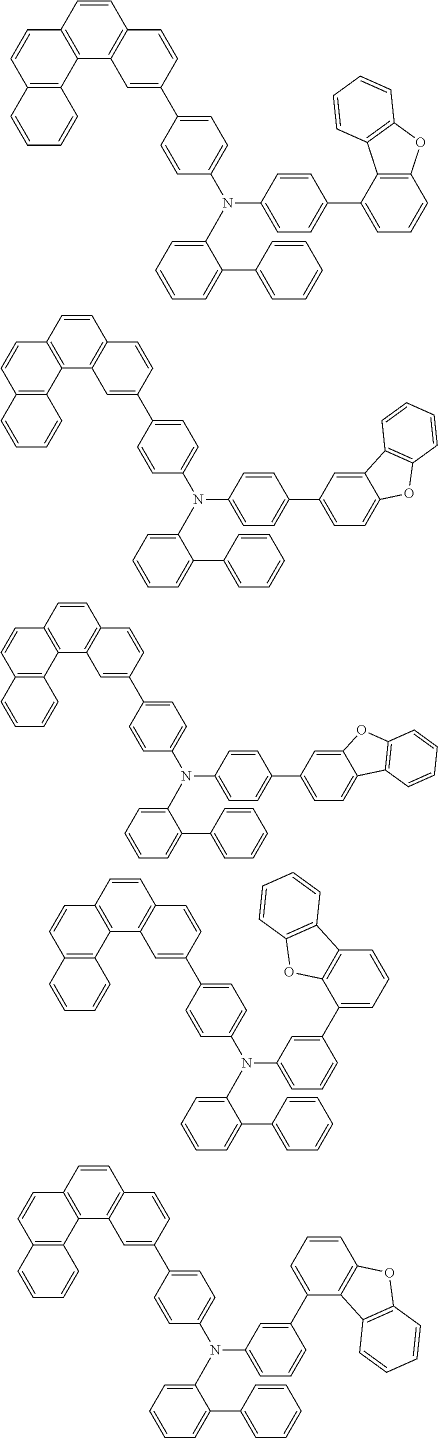

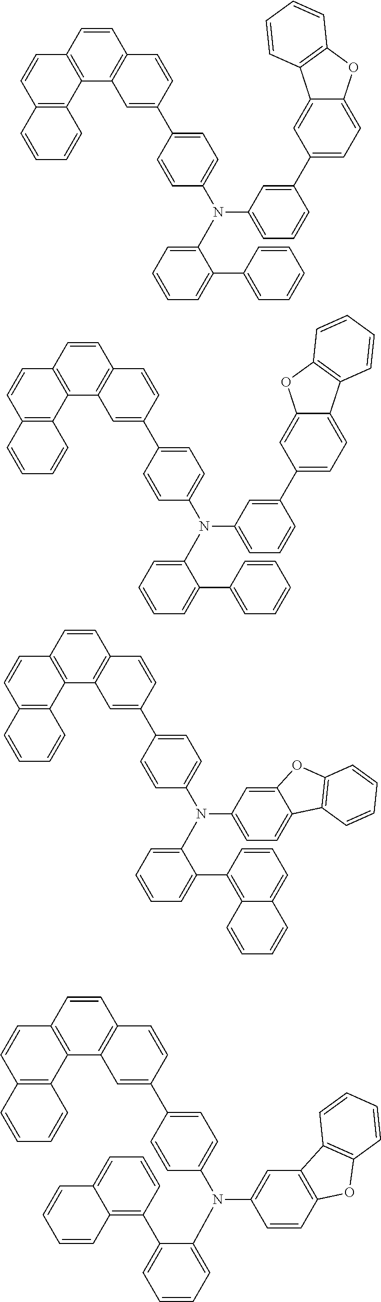

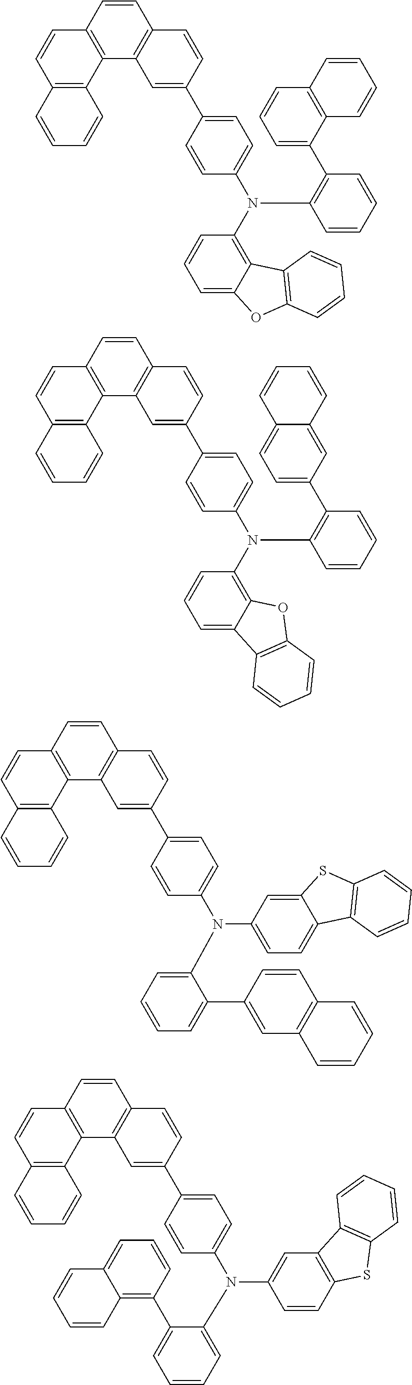 [SMILES: c1ccc(N(c2ccc(-c3ccc4ccc5ccc6ccccc6c5c4c3)cc2)c2cccc3oc4ccccc4c23)c(-c2cccc3ccccc23)c1 ; c1ccc(N(c2ccc(-c3ccc4ccc5ccc6ccccc6c5c4c3)cc2)c2cccc3c2oc2ccccc23)c(-c2ccc3ccccc3c2)c1 ; c1ccc(N(c2ccc(-c3ccc4ccc5ccc6ccccc6c5c4c3)cc2)c2ccc3c(c2)sc2ccccc23)c(-c2ccc3ccccc3c2)c1 ; c1ccc(N(c2ccc(-c3ccc4ccc5ccc6ccccc6c5c4c3)cc2)c2ccc3sc4ccccc4c3c2)c(-c2cccc3ccccc23)c1]

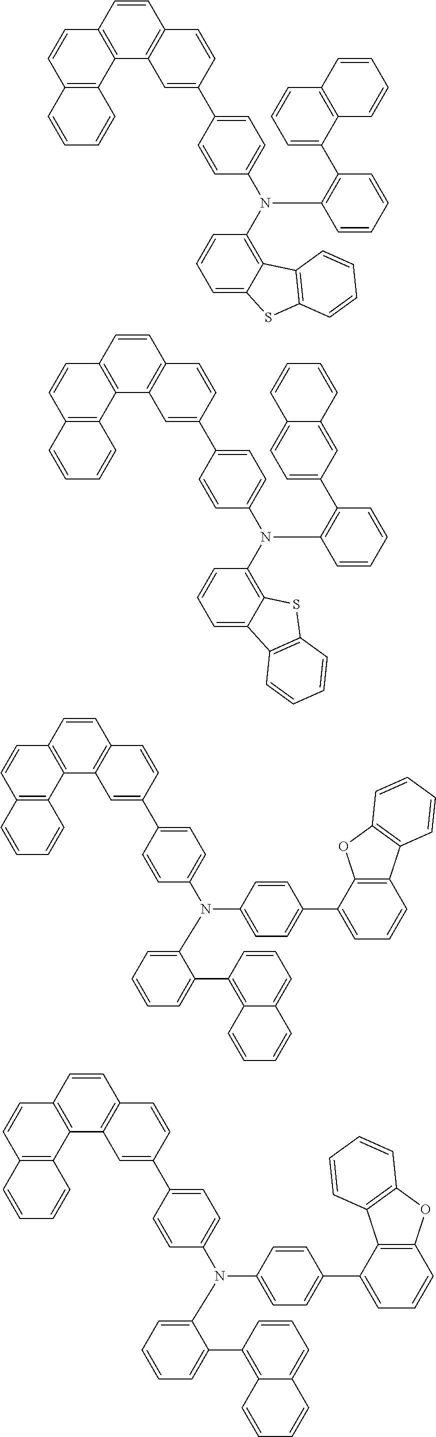 [SMILES: c1ccc(N(c2ccc(-c3ccc4ccc5ccc6ccccc6c5c4c3)cc2)c2cccc3sc4ccccc4c23)c(-c2cccc3ccccc23)c1 ; c1ccc(N(c2ccc(-c3ccc4ccc5ccc6ccccc6c5c4c3)cc2)c2cccc3c2sc2ccccc23)c(-c2ccc3ccccc3c2)c1 ; c1ccc(N(c2ccc(-c3ccc4ccc5ccc6ccccc6c5c4c3)cc2)c2ccc(-c3cccc4c3oc3ccccc34)cc2)c(-c2cccc3ccccc23)c1 ; c1ccc(N(c2ccc(-c3ccc4ccc5ccc6ccccc6c5c4c3)cc2)c2ccc(-c3cccc4oc5ccccc5c34)cc2)c(-c2cccc3ccccc23)c1]

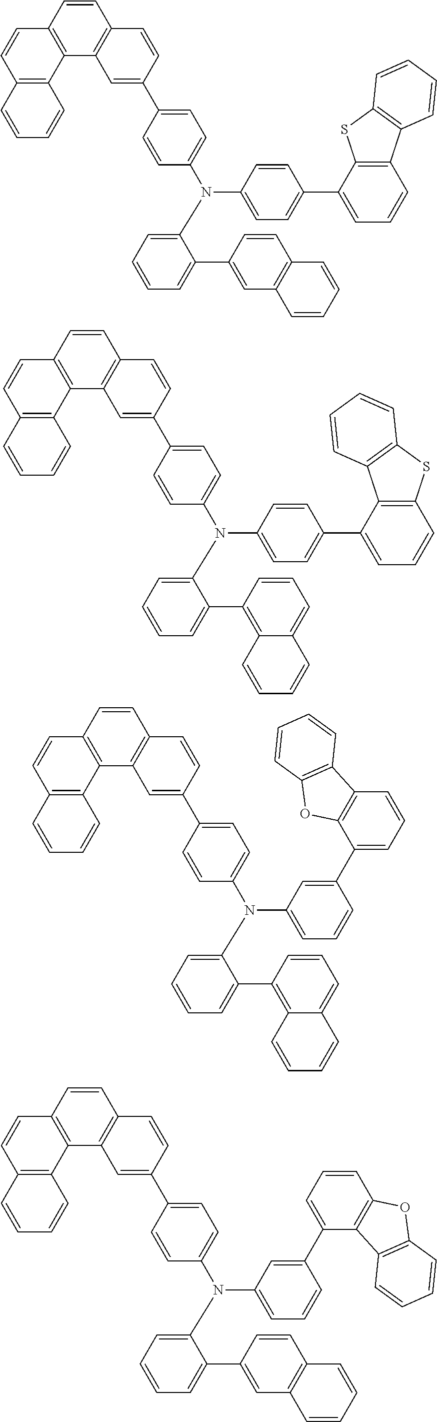

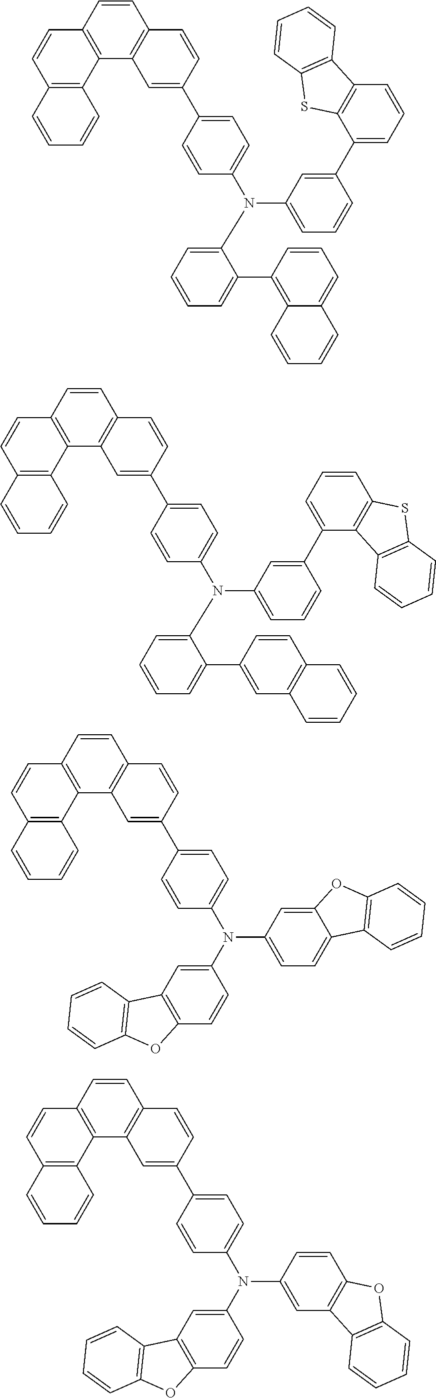

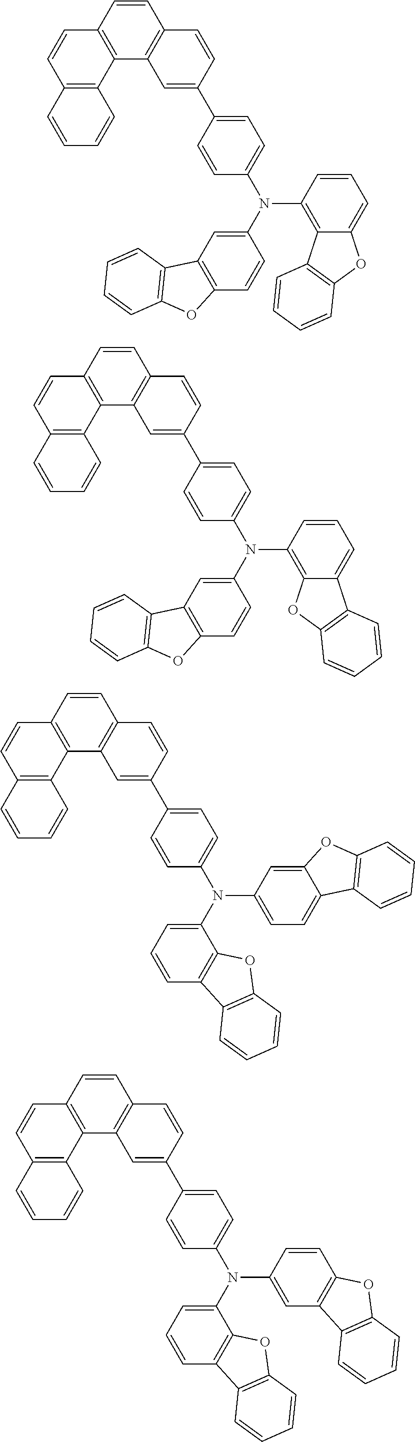

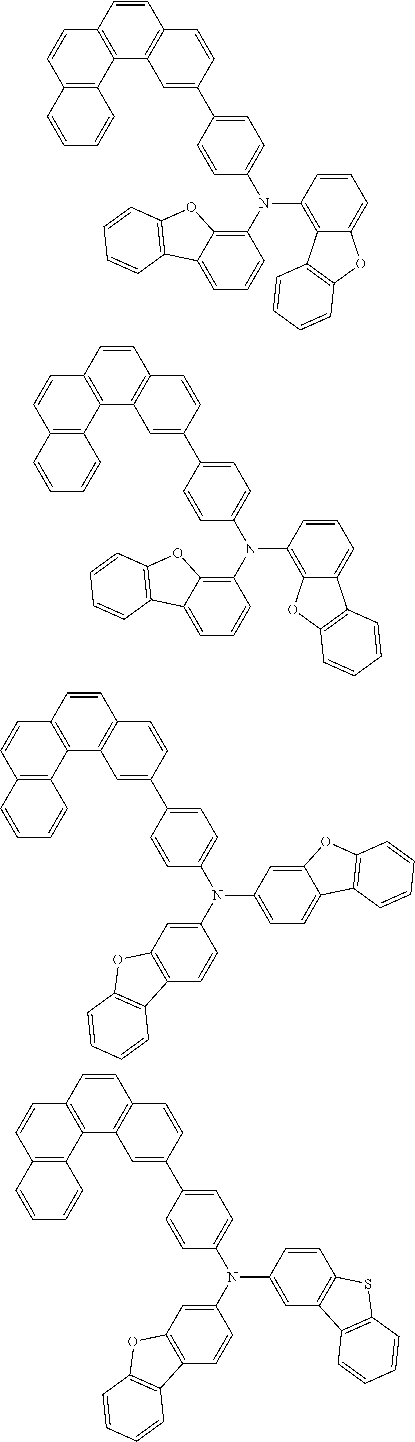

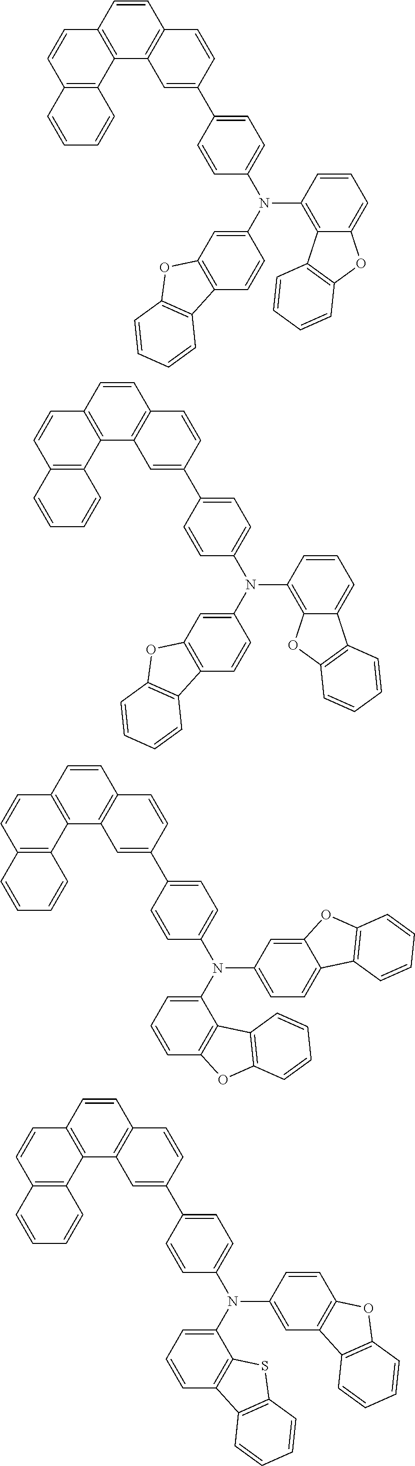

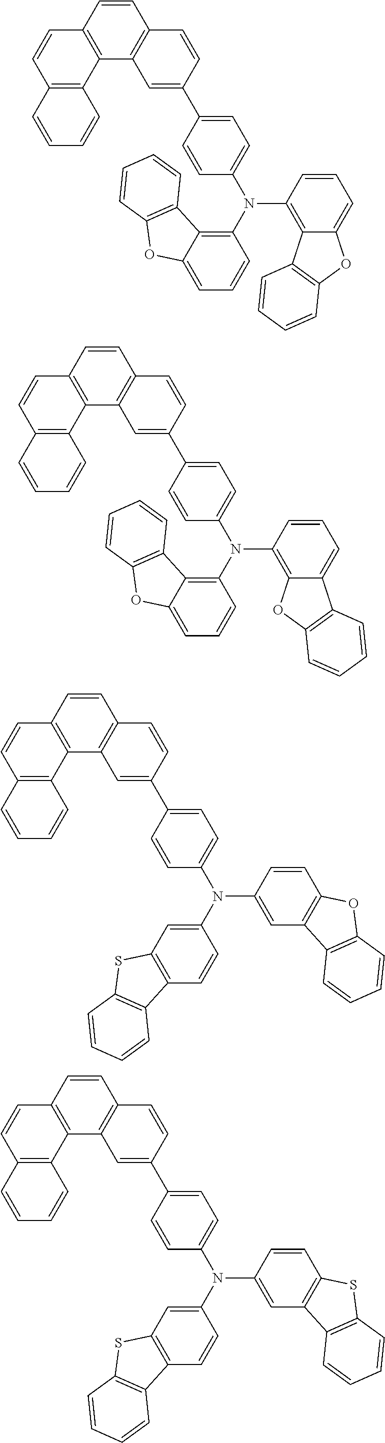

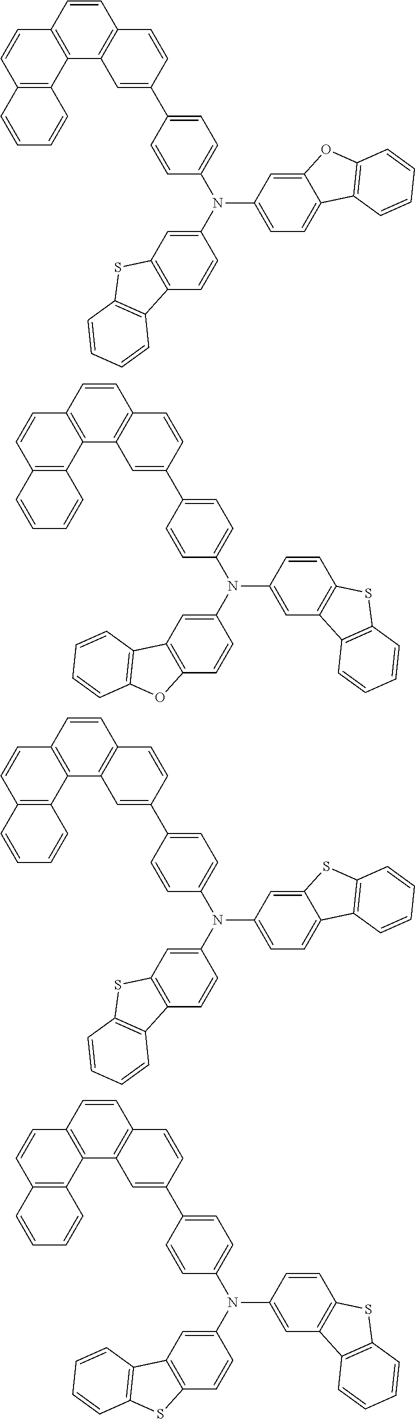

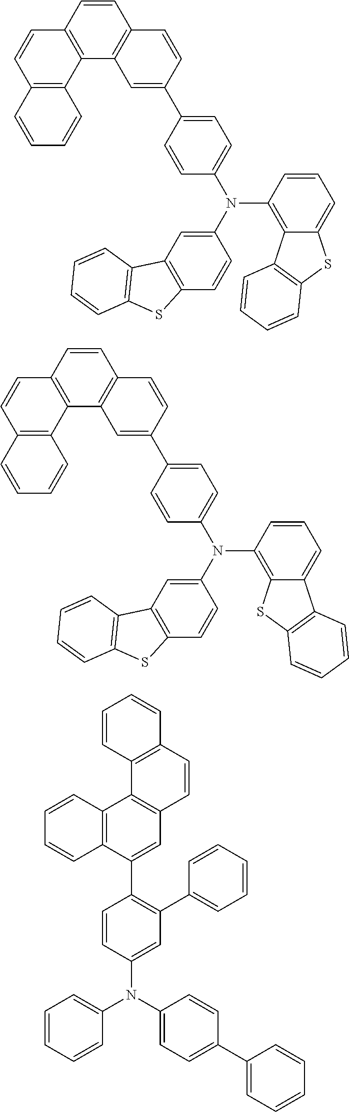

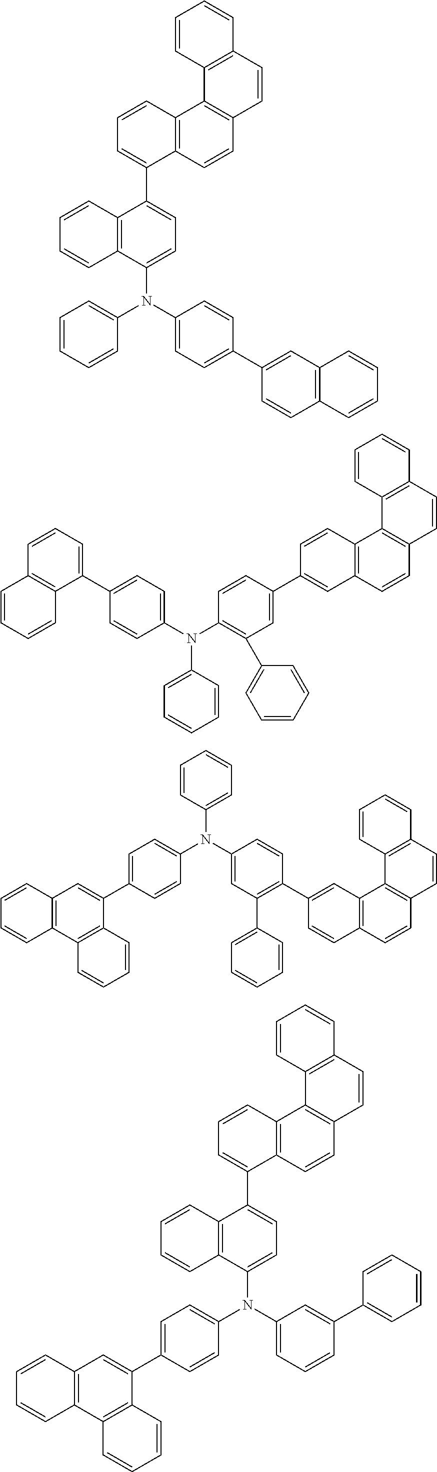

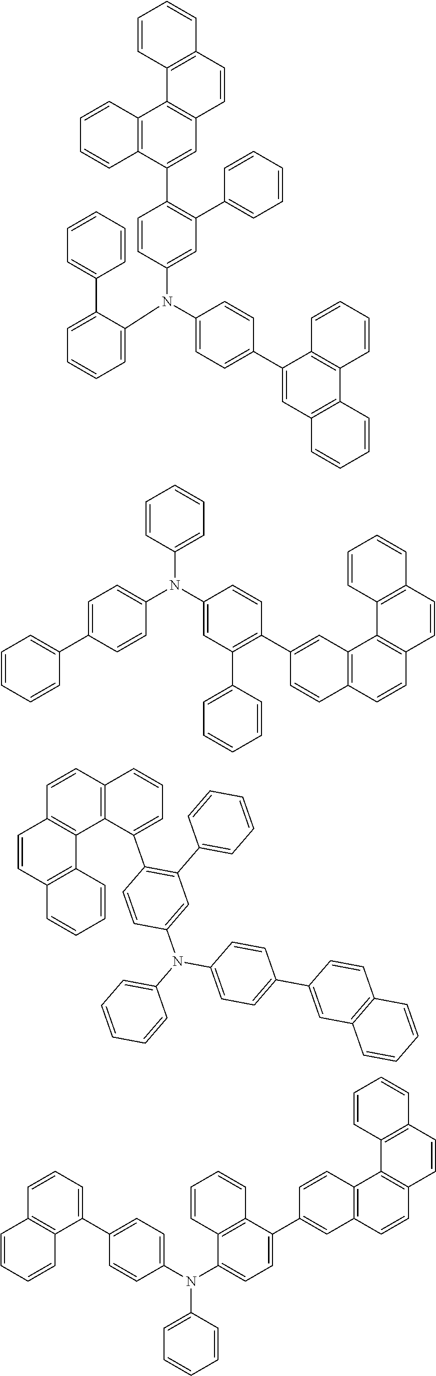

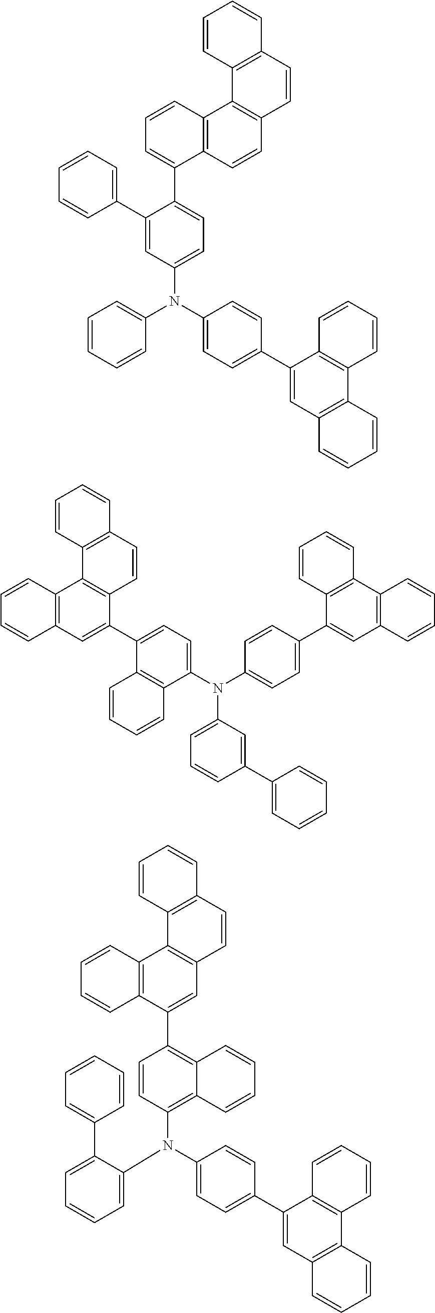 [SMILES: c1ccc(-c2cc(N(c3ccccc3)c3ccc(-c4cc5ccccc5c5ccccc45)cc3)ccc2-c2cccc3c2ccc2ccc4ccccc4c23)cc1 ; c1ccc(-c2cccc(N(c3ccc(-c4cc5ccccc5c5ccccc45)cc3)c3ccc(-c4cc5ccccc5c5c4ccc4ccccc45)c4ccccc34)c2)cc1 ; c1ccc(-c2ccccc2N(c2ccc(-c3cc4ccccc4c4ccccc34)cc2)c2ccc(-c3cc4ccc5ccccc5c4c4ccccc34)c3ccccc23)cc1]

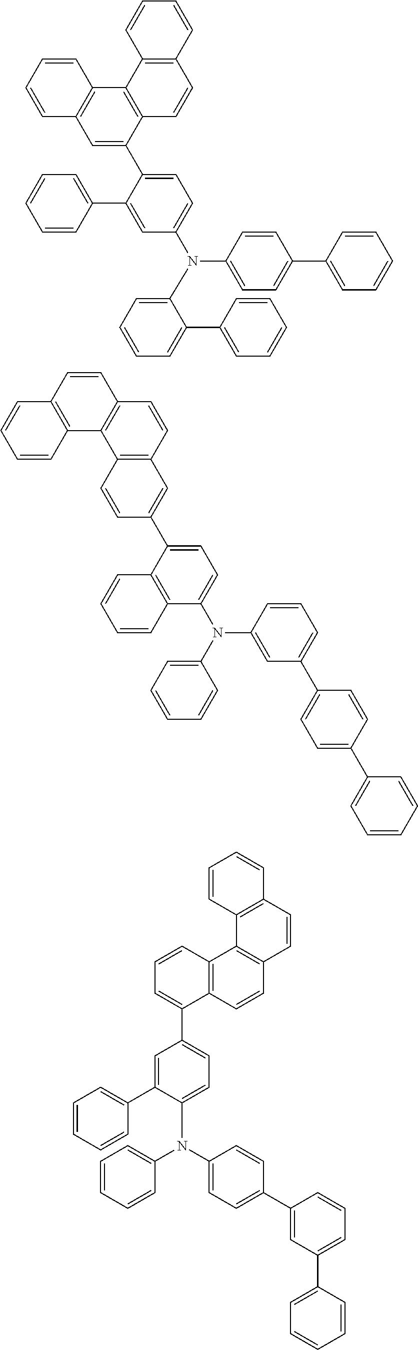

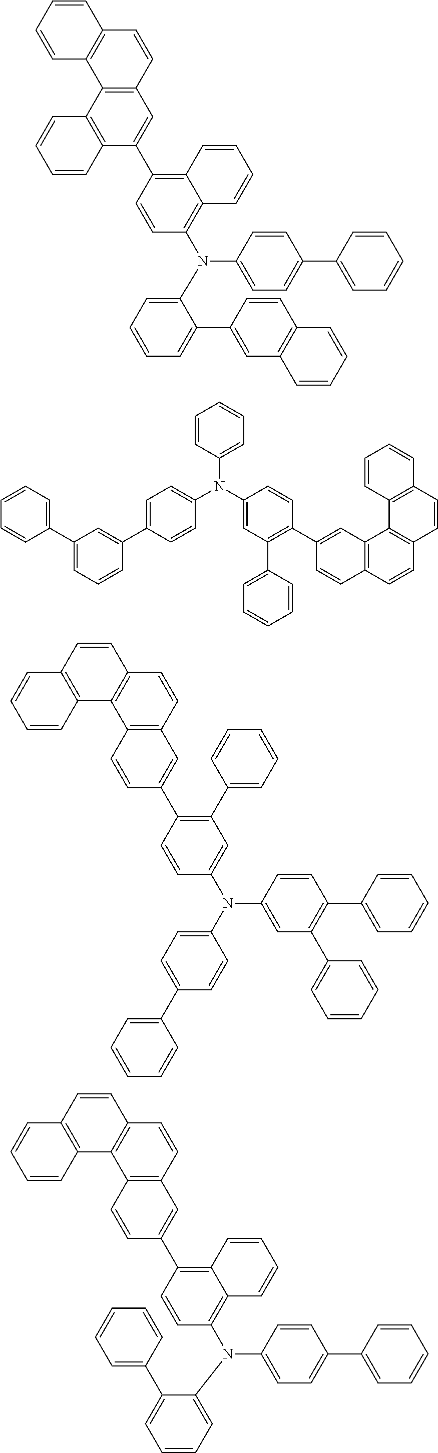

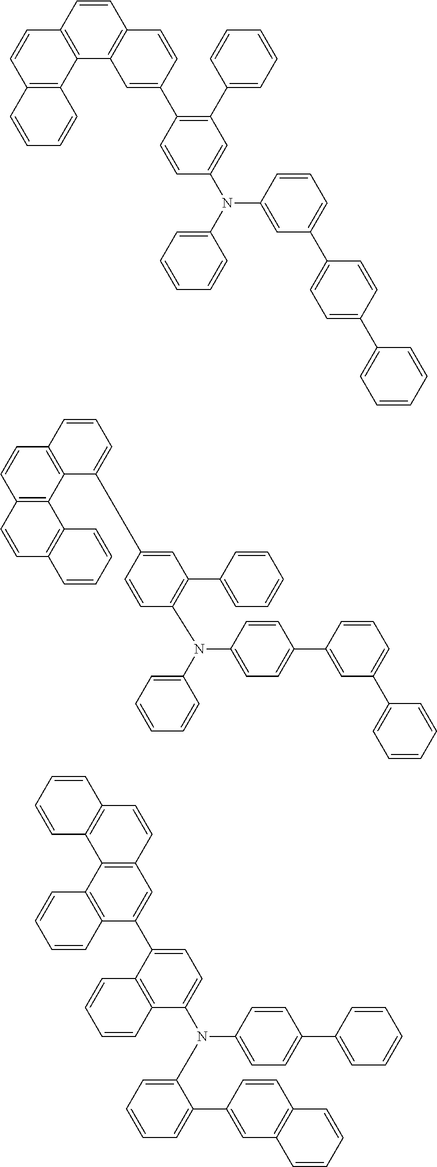 [SMILES: c1ccc(-c2ccc(-c3cccc(N(c4ccccc4)c4ccc(-c5ccc6ccc7ccc8ccccc8c7c6c5)c(-c5ccccc5)c4)c3)cc2)cc1 ; c1ccc(-c2cccc(-c3ccc(N(c4ccccc4)c4ccc(-c5cccc6ccc7ccc8ccccc8c7c56)cc4-c4ccccc4)cc3)c2)cc1 ; c1ccc(-c2ccc(N(c3ccccc3-c3ccc4ccccc4c3)c3ccc(-c4cc5ccc6ccccc6c5c5ccccc45)c4ccccc34)cc2)cc1]

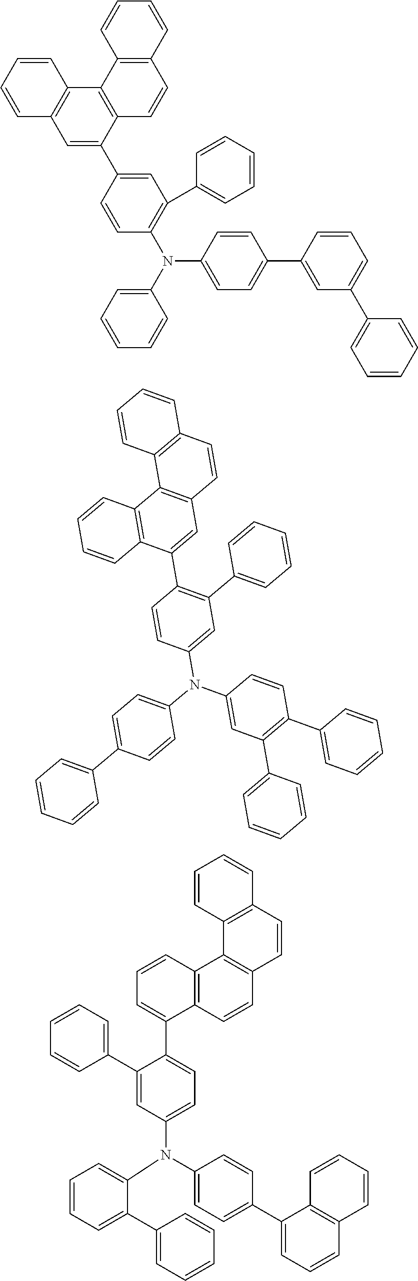

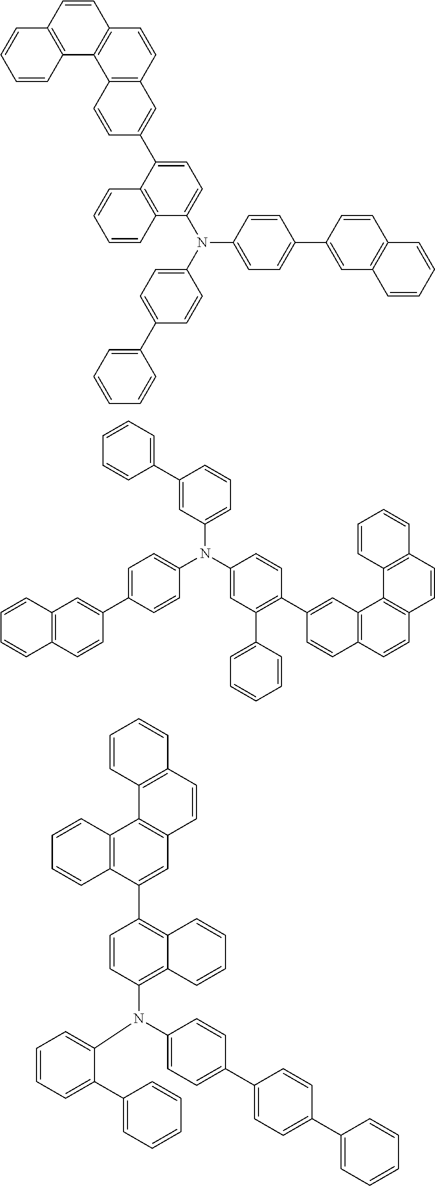

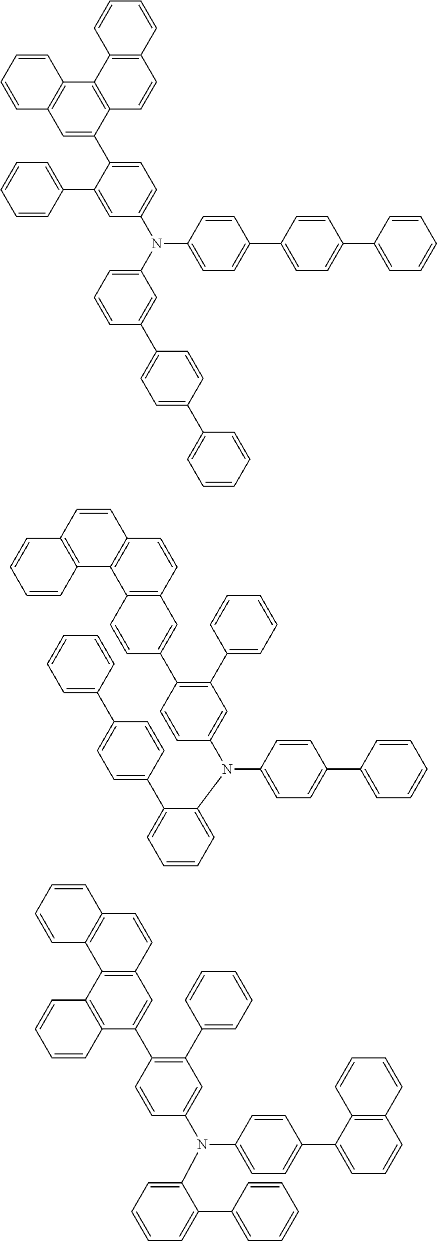 [SMILES: c1ccc(-c2ccc(-c3ccc(N(c4cccc(-c5ccc(-c6ccccc6)cc5)c4)c4ccc(-c5cc6ccccc6c6c5ccc5ccccc56)c(-c5ccccc5)c4)cc3)cc2)cc1 ; c1ccc(-c2ccc(-c3ccccc3N(c3ccc(-c4ccccc4)cc3)c3ccc(-c4ccc5c(ccc6ccc7ccccc7c65)c4)c(-c4ccccc4)c3)cc2)cc1 ; c1ccc(-c2cc(N(c3ccc(-c4cccc5ccccc45)cc3)c3ccccc3-c3ccccc3)ccc2-c2cc3ccc4ccccc4c3c3ccccc23)cc1]

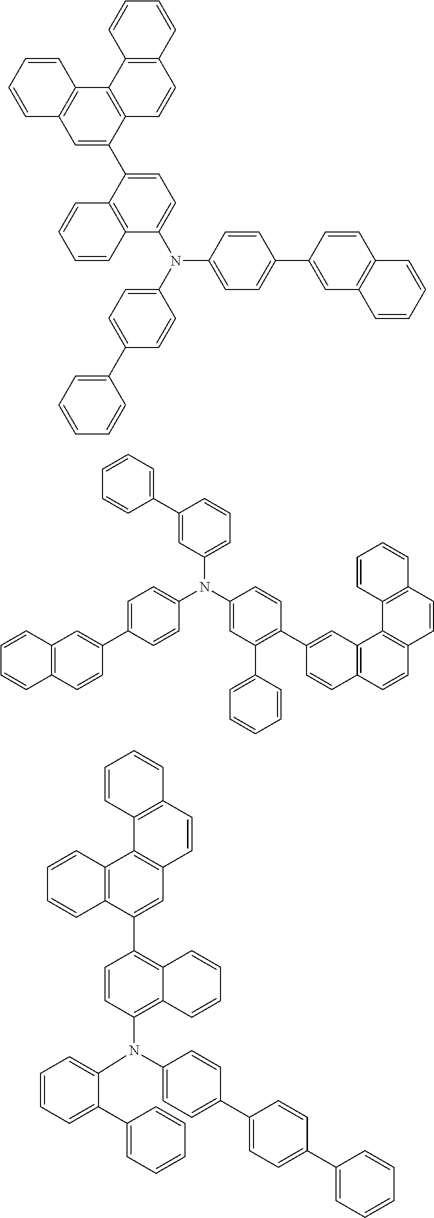

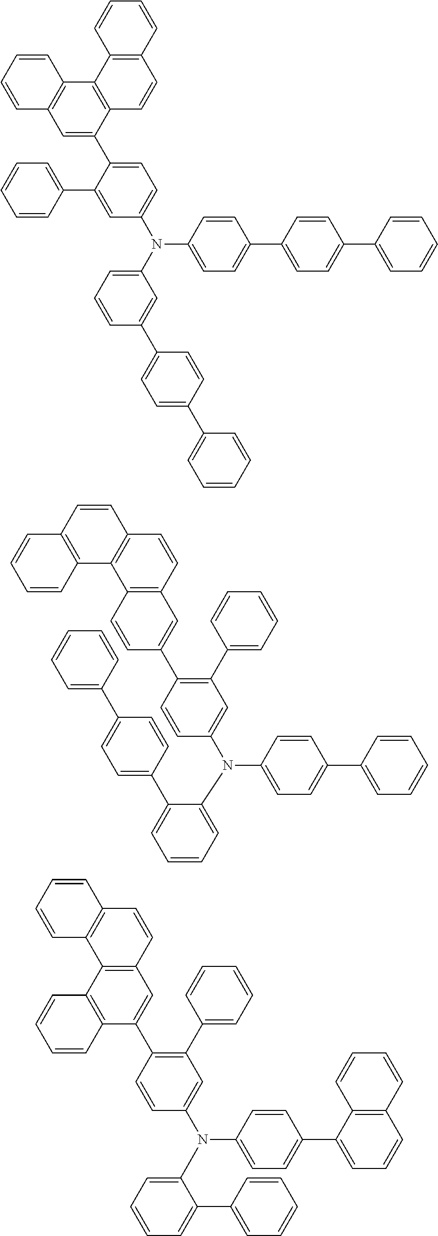 [SMILES: c1ccc(-c2ccc(-c3ccc(N(c4cccc(-c5ccc(-c6ccccc6)cc5)c4)c4ccc(-c5cc6ccccc6c6c5ccc5ccccc56)c(-c5ccccc5)c4)cc3)cc2)cc1 ; c1ccc(-c2ccc(-c3ccccc3N(c3ccc(-c4ccccc4)cc3)c3ccc(-c4ccc5c(ccc6ccc7ccccc7c65)c4)c(-c4ccccc4)c3)cc2)cc1 ; c1ccc(-c2cc(N(c3ccc(-c4cccc5ccccc45)cc3)c3ccccc3-c3ccccc3)ccc2-c2cc3ccc4ccccc4c3c3ccccc23)cc1]

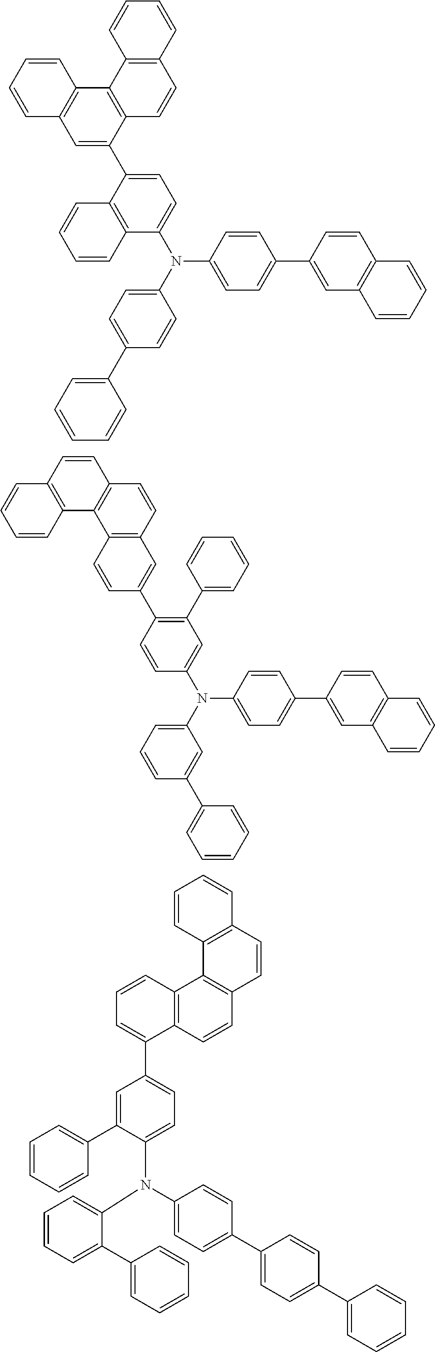

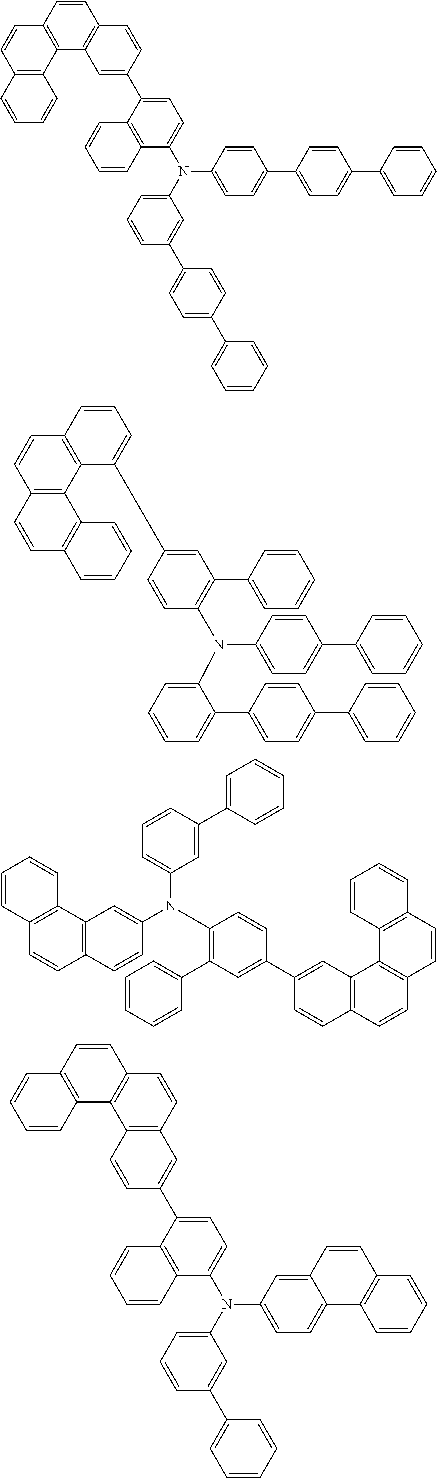

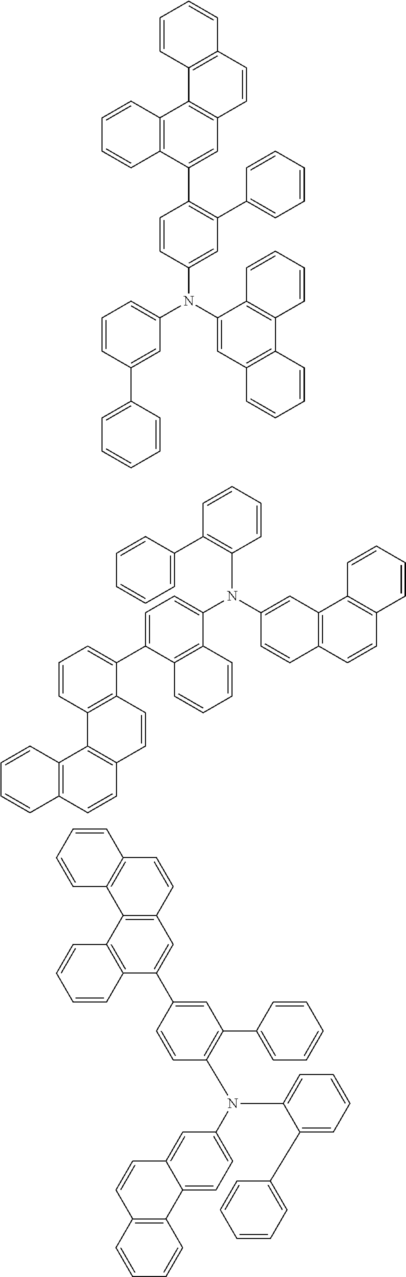

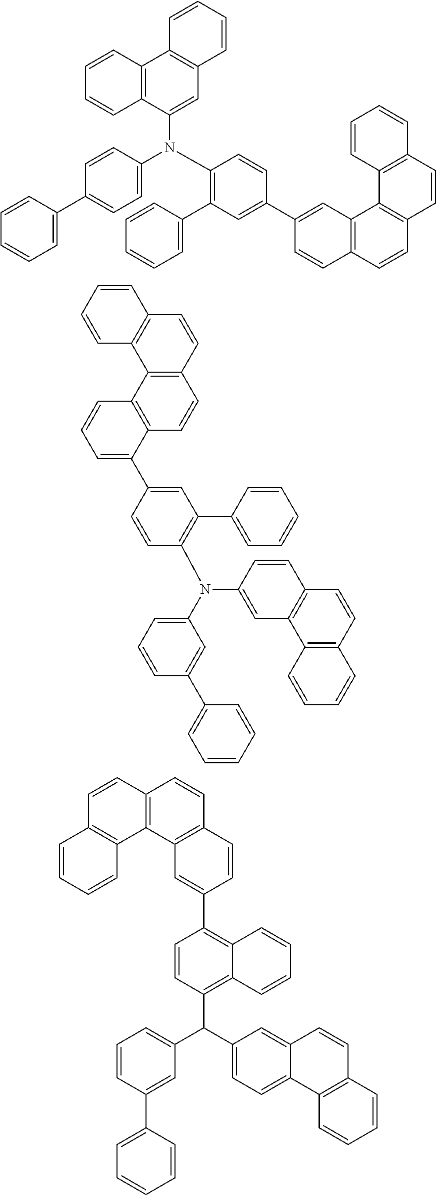

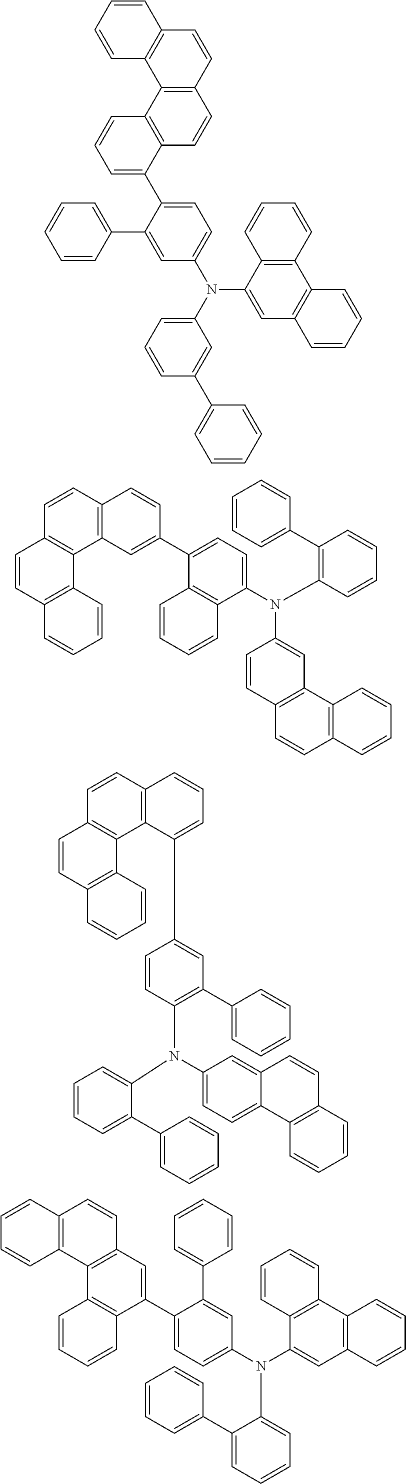

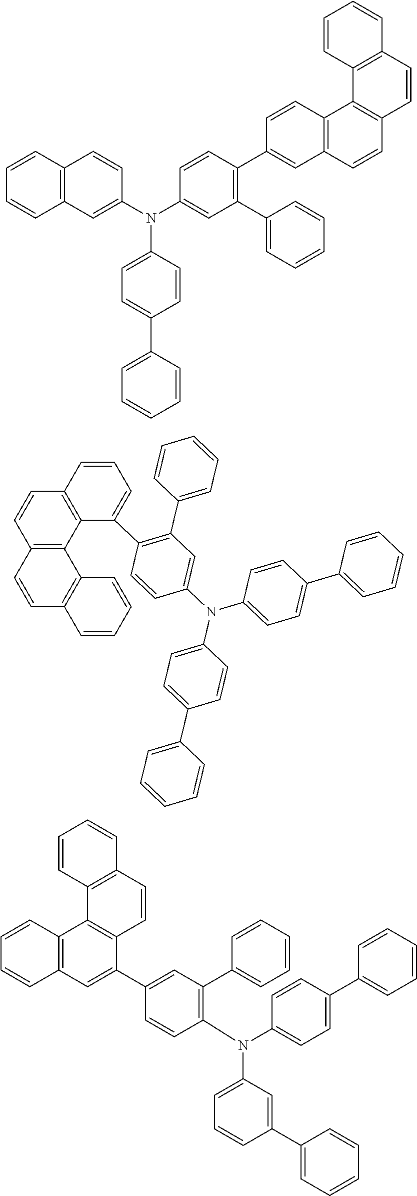

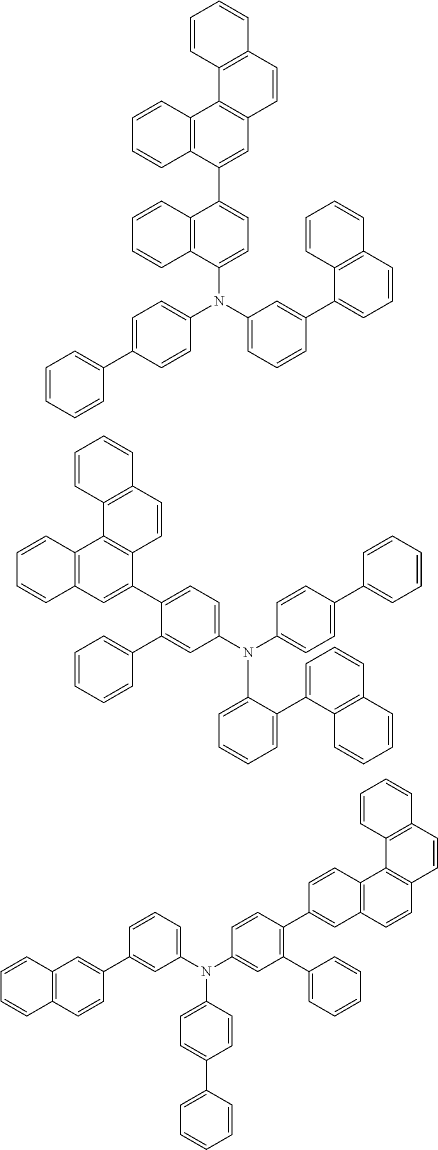

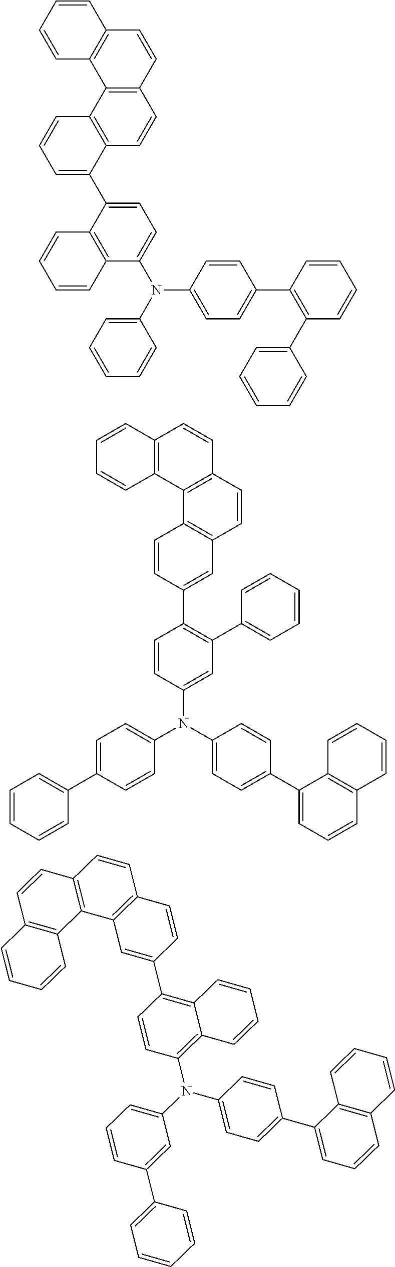

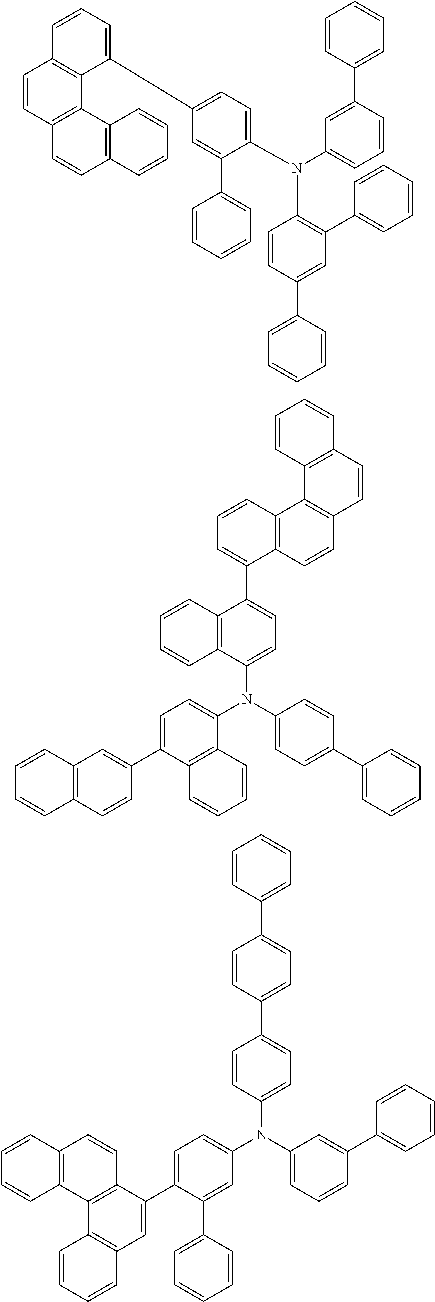

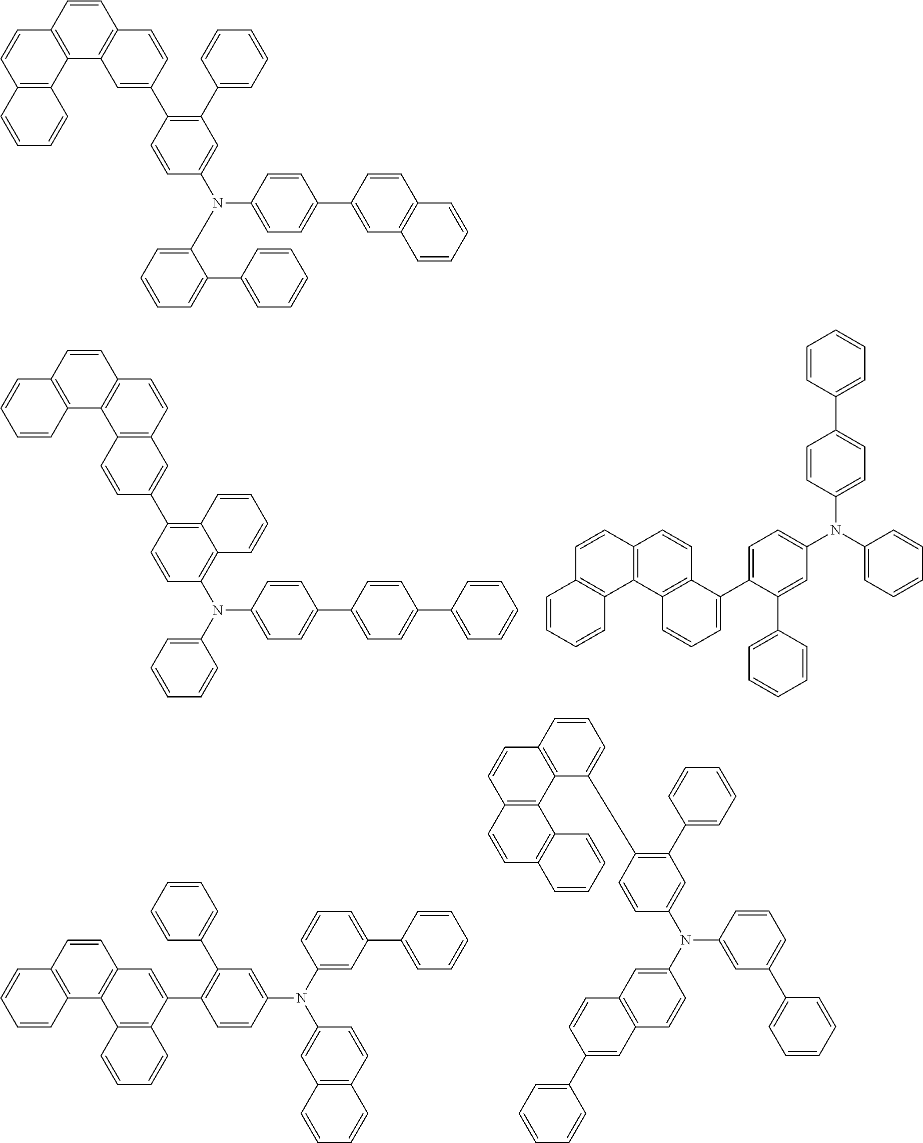

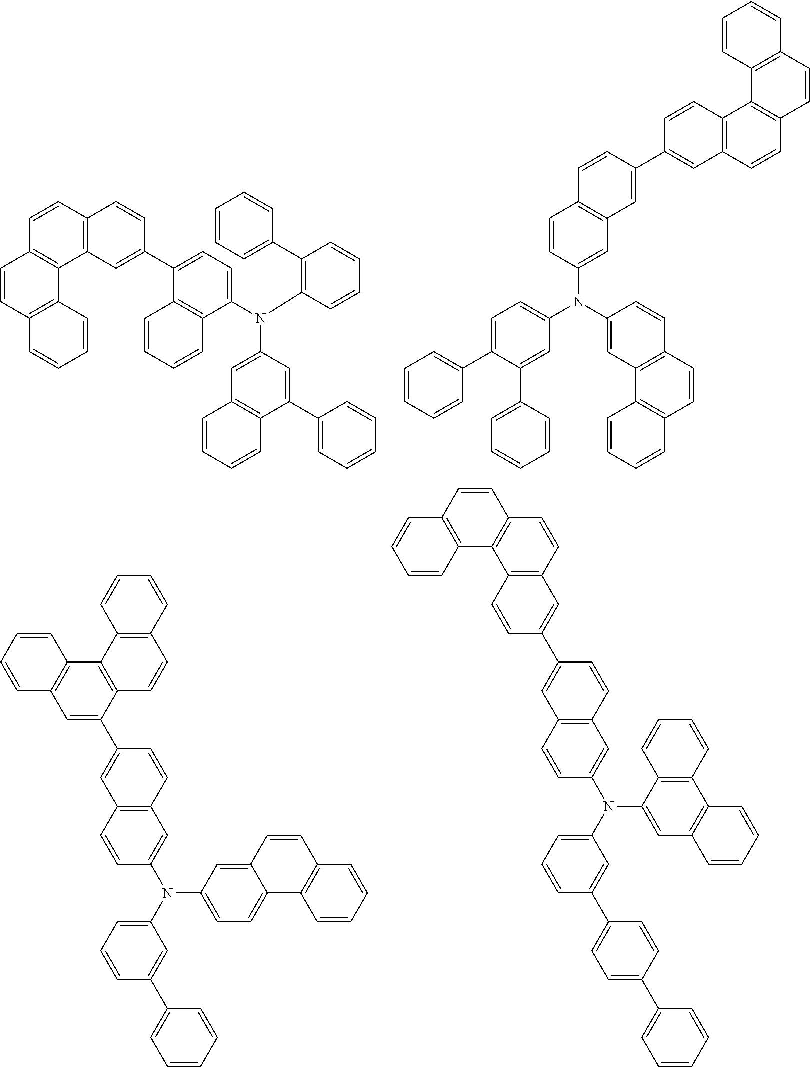

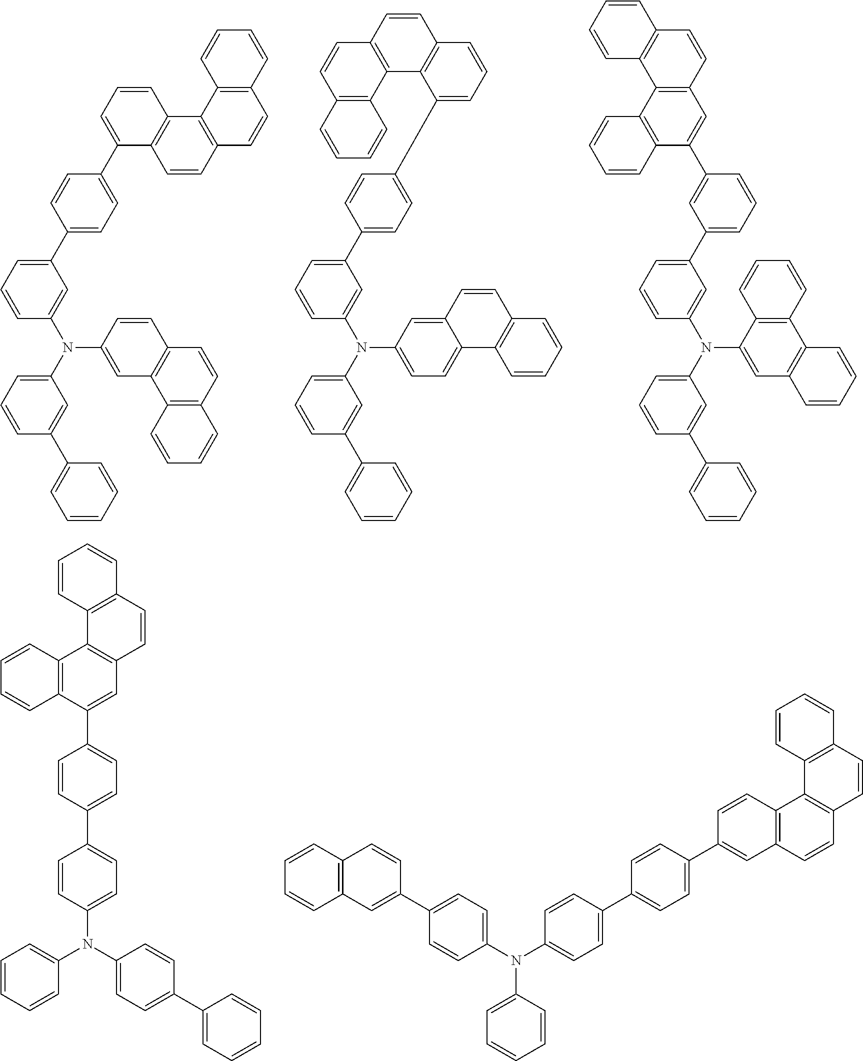

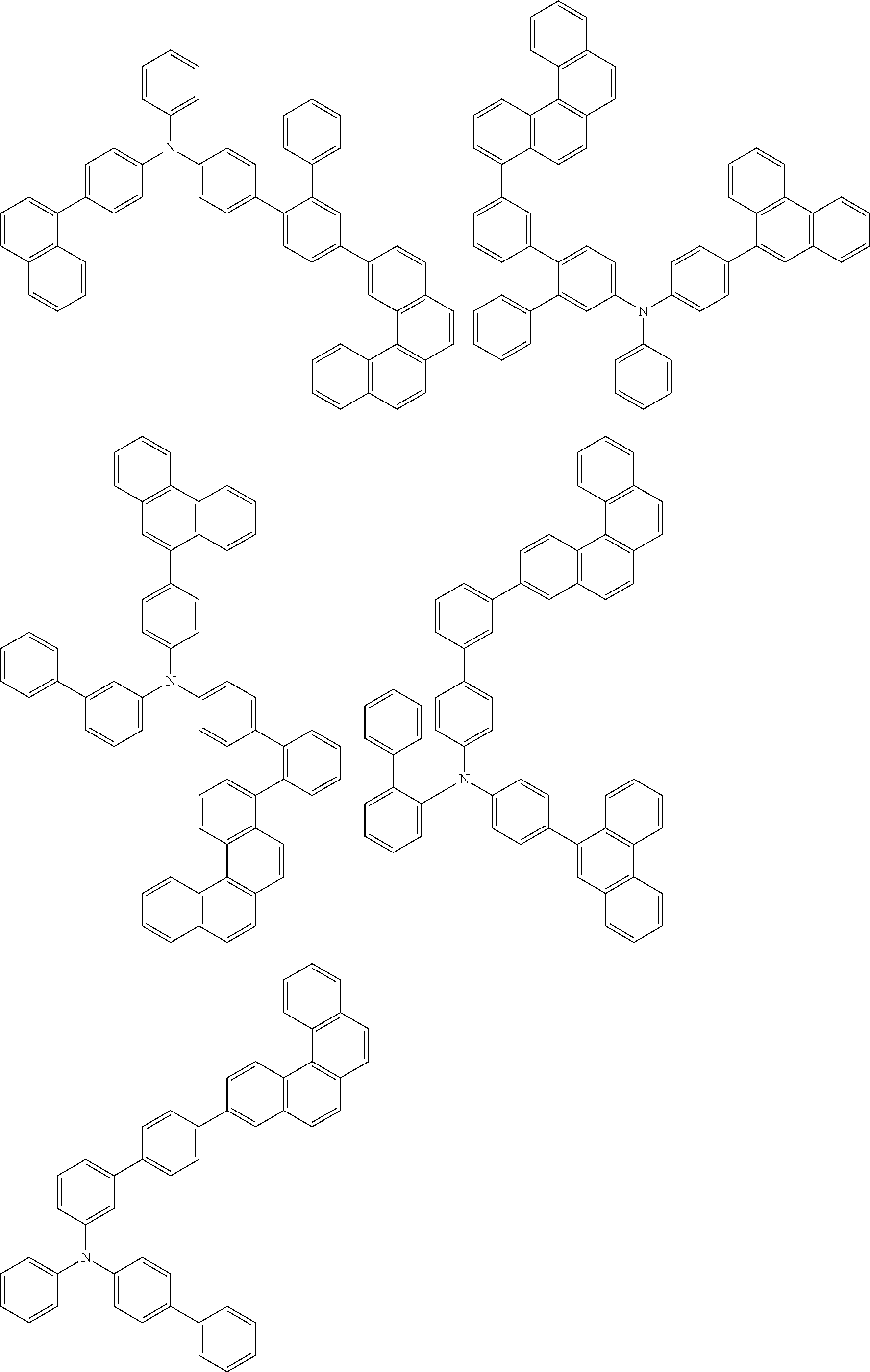

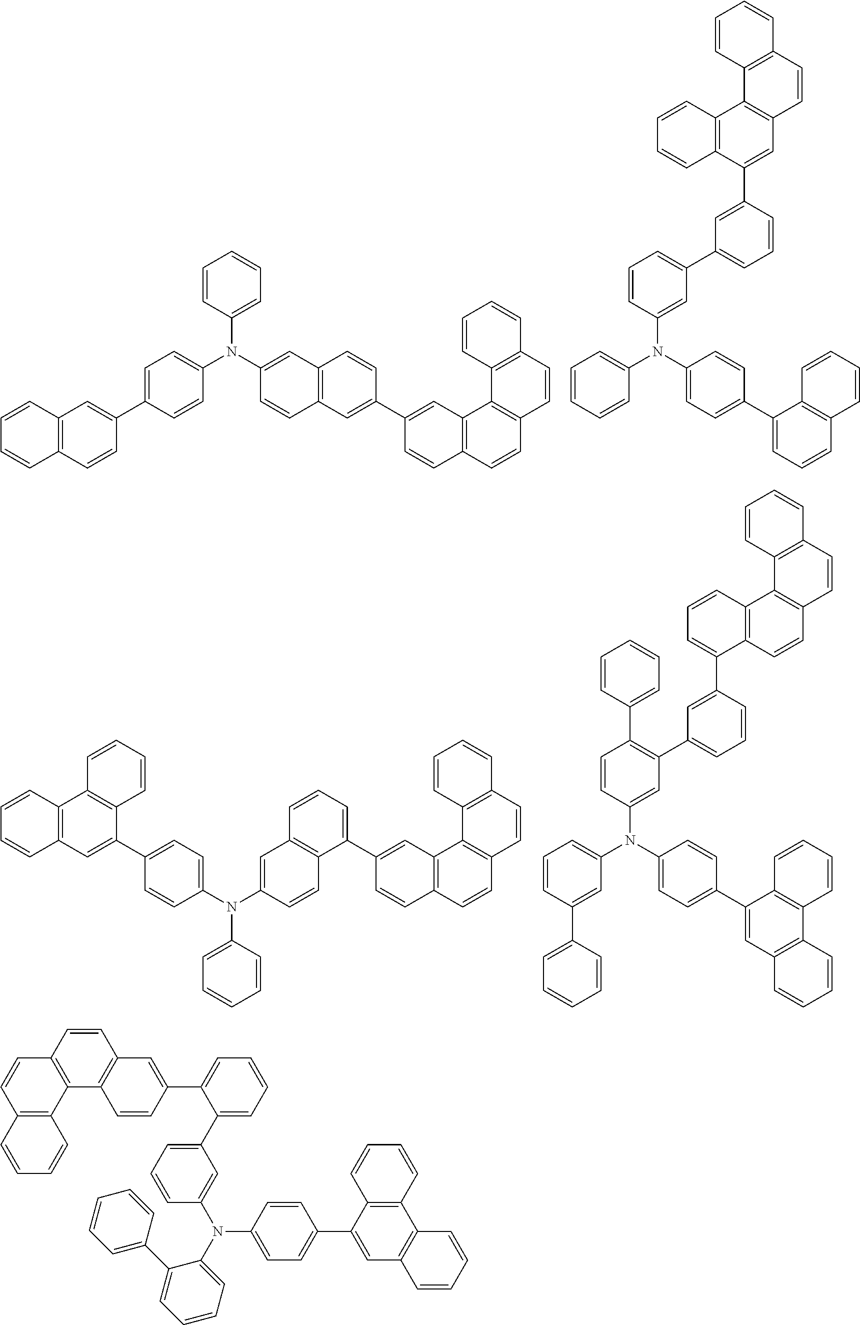

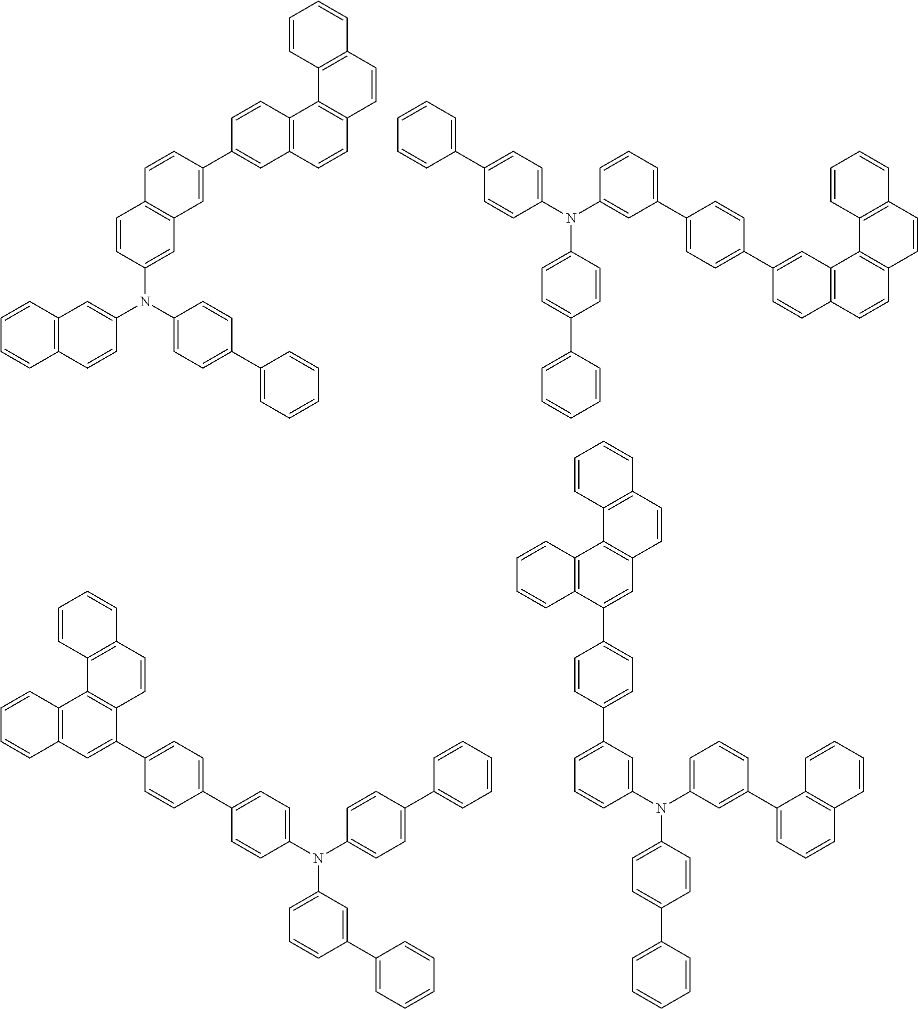

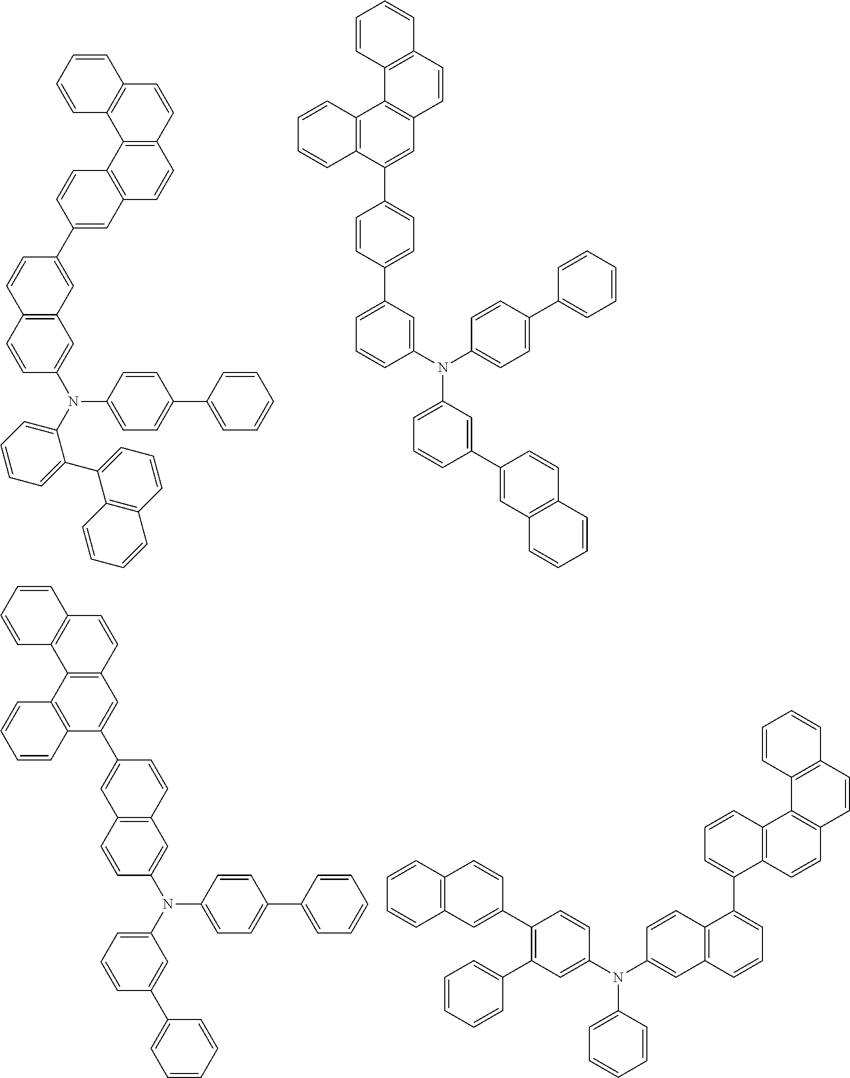

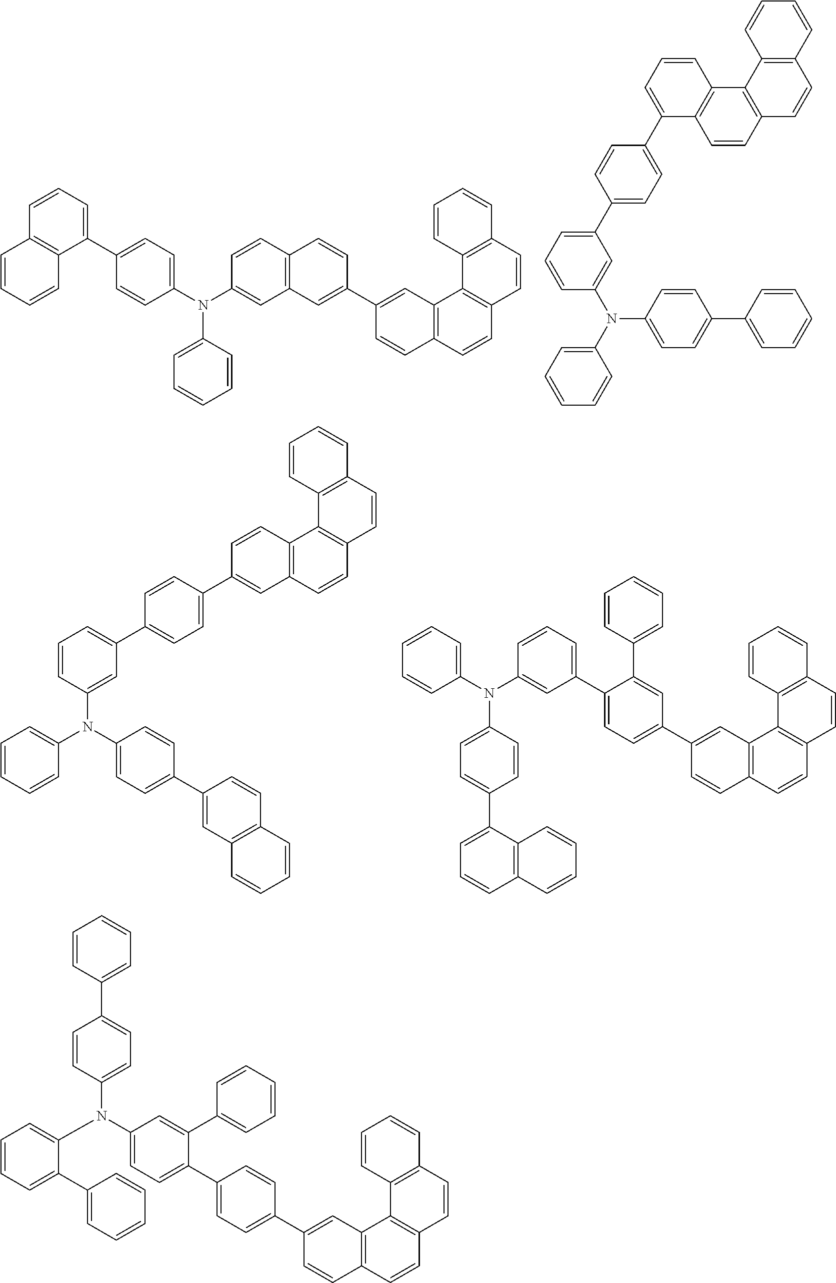

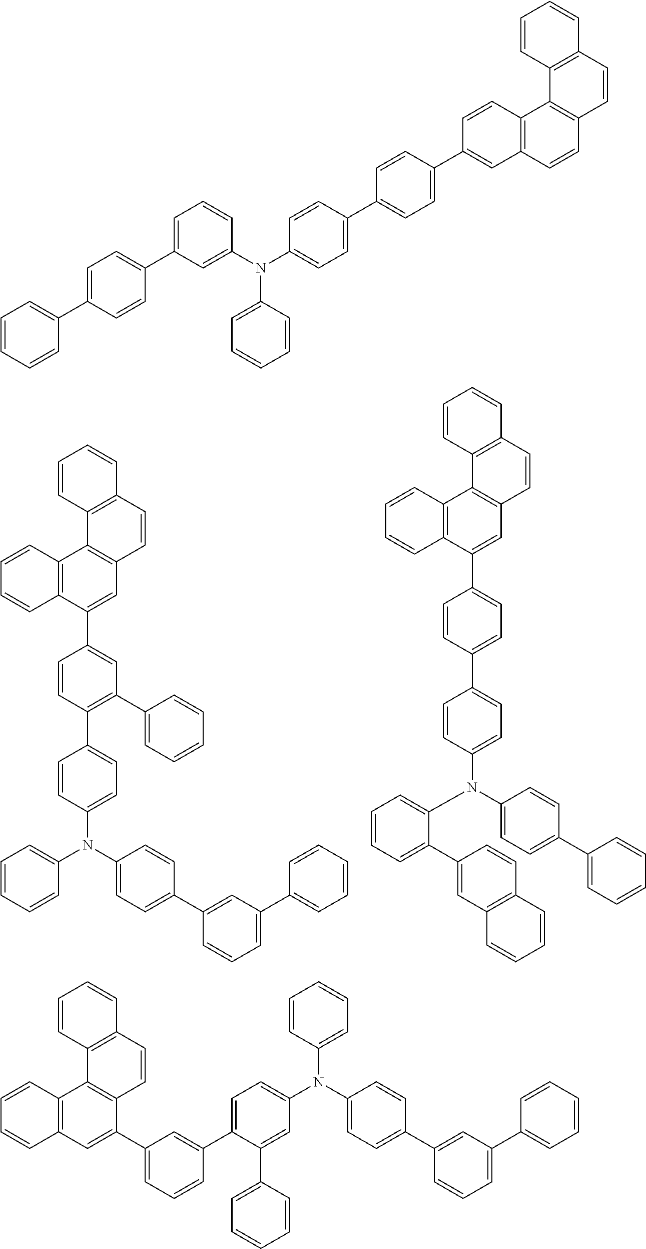

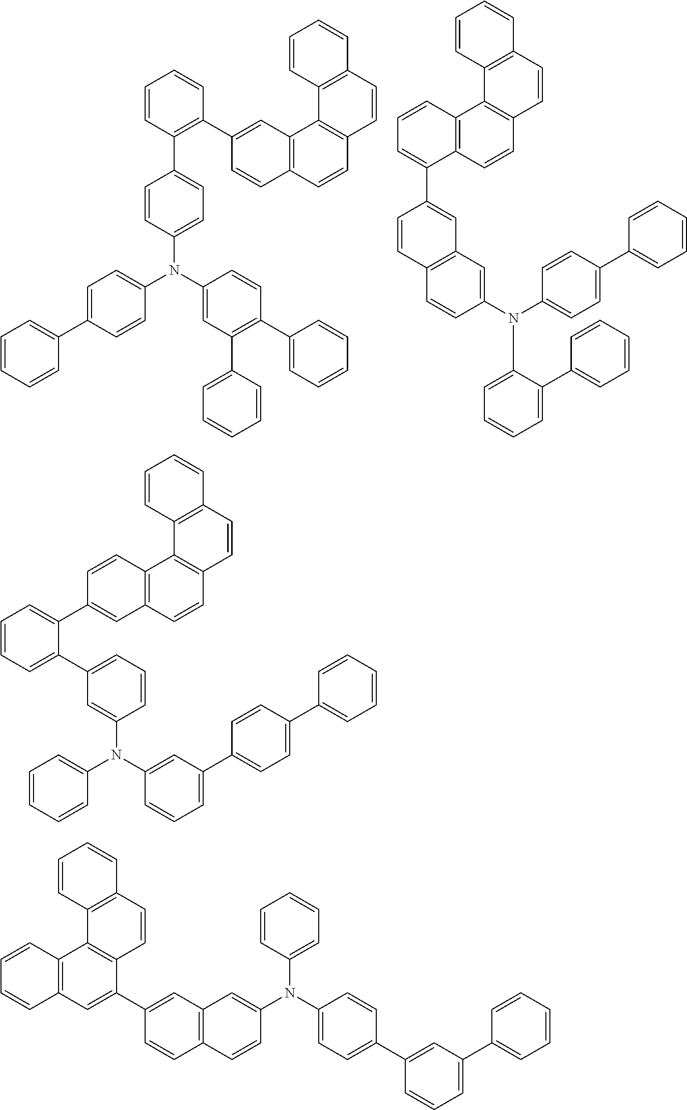

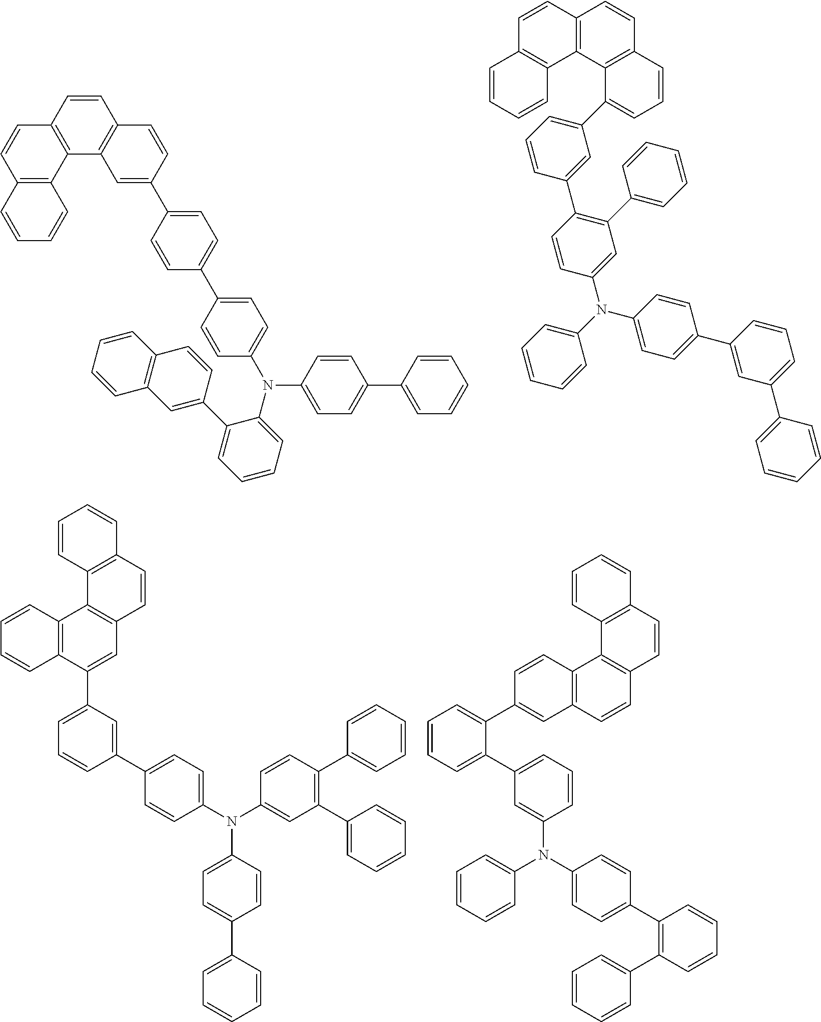

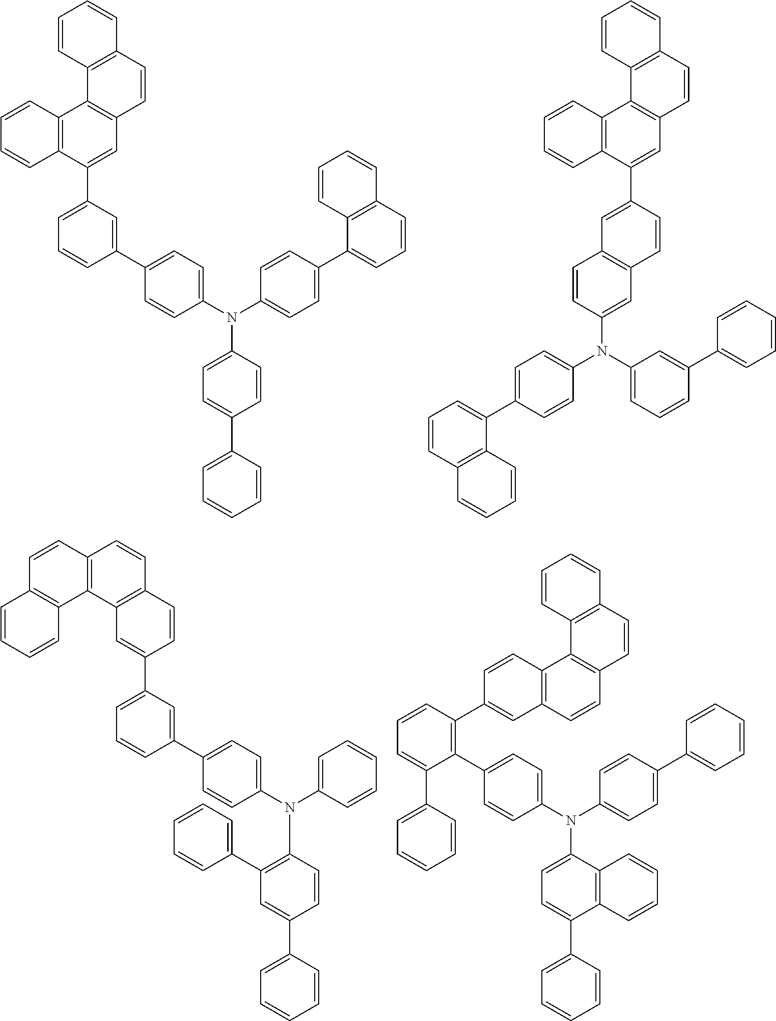

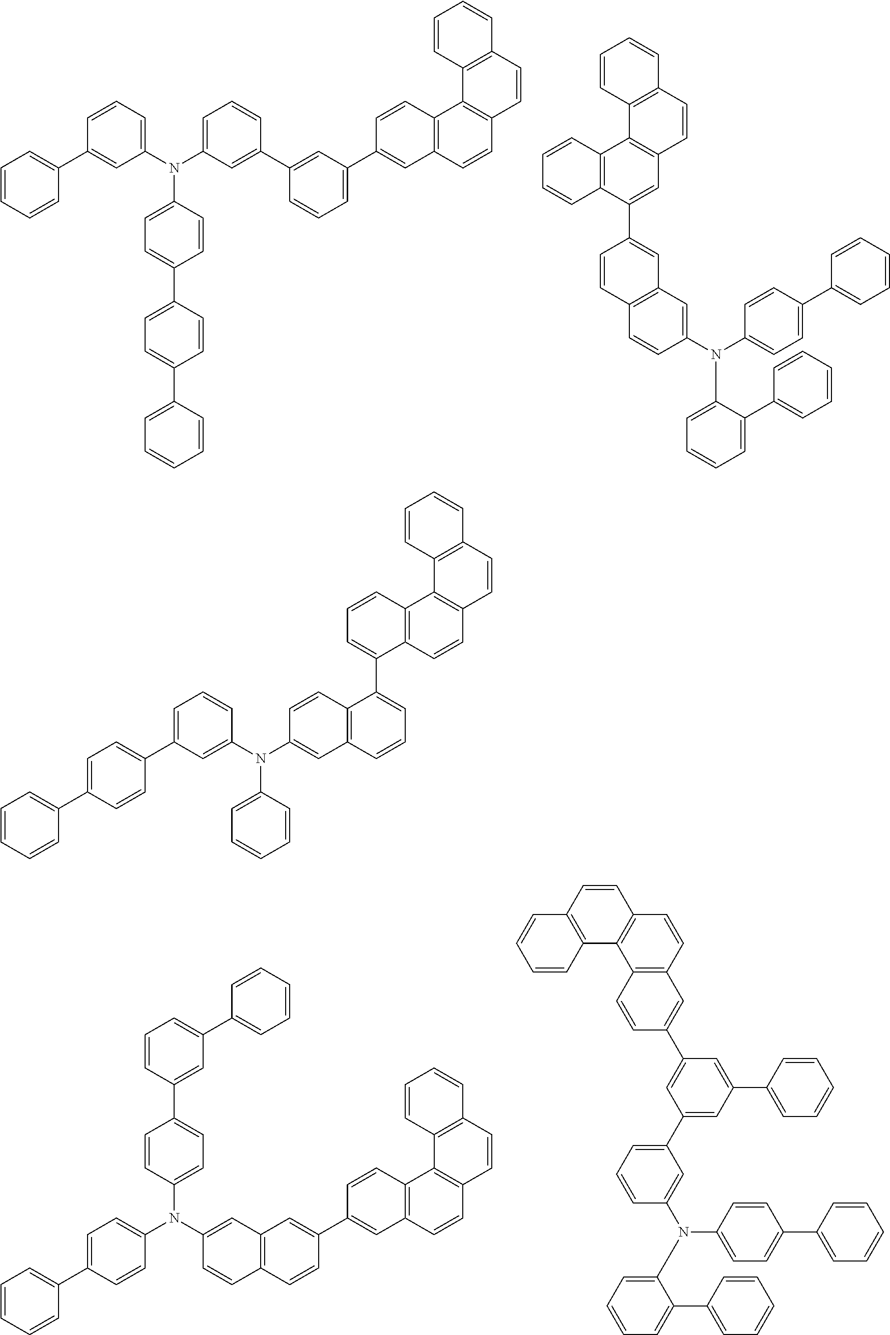 [SMILES: c1ccc(-c2ccc(-c3ccc(N(c4cccc(-c5ccccc5)c4)c4cccc(-c5cccc(-c6ccc7c(ccc8ccc9ccccc9c87)c6)c5)c4)cc3)cc2)cc1 ; c1ccc(-c2ccc(N(c3ccc4ccc(-c5cc6ccc7ccccc7c6c6ccccc56)cc4c3)c3ccccc3-c3ccccc3)cc2)cc1 ; c1ccc(-c2ccc(-c3cccc(N(c4ccccc4)c4ccc5c(-c6cccc7c6ccc6ccc8ccccc8c67)cccc5c4)c3)cc2)cc1 ; c1ccc(-c2ccc(N(c3ccc(-c4cccc(-c5ccccc5)c4)cc3)c3ccc4ccc(-c5ccc6c(ccc7ccc8ccccc8c76)c5)cc4c3)cc2)cc1 ; c1ccc(-c2ccc(N(c3cccc(-c4cc(-c5ccccc5)cc(-c5ccc6c(ccc7ccc8ccccc8c76)c5)c4)c3)c3ccccc3-c3ccccc3)cc2)cc1]

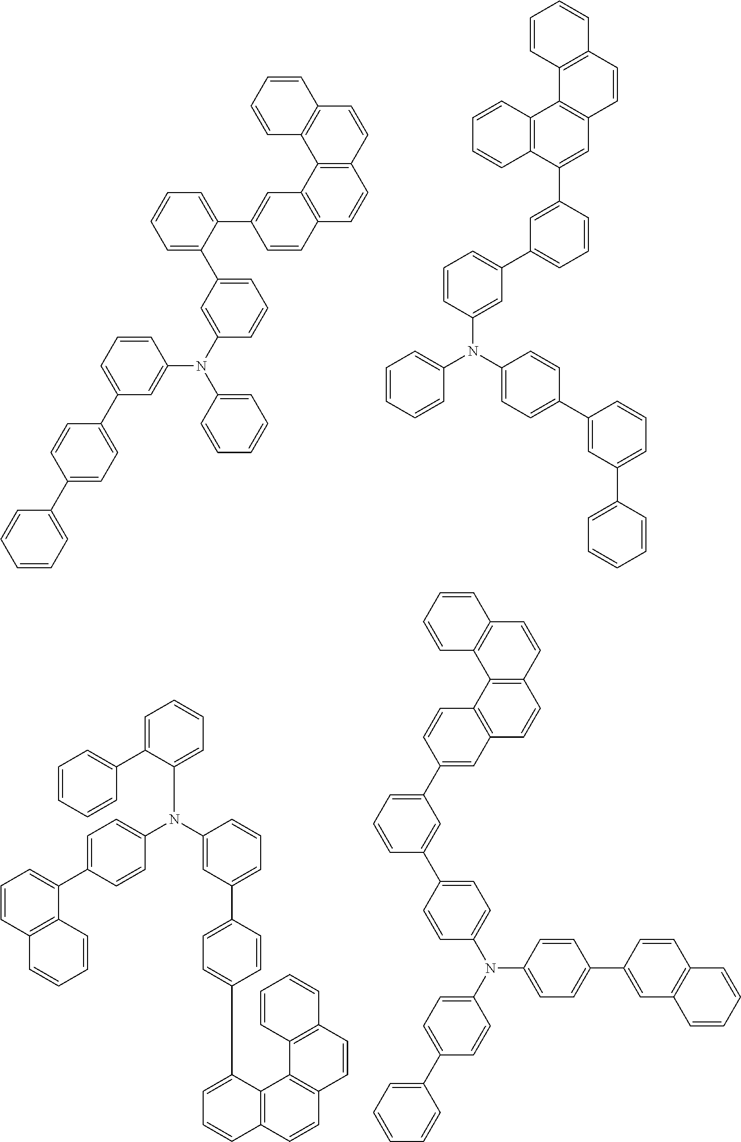

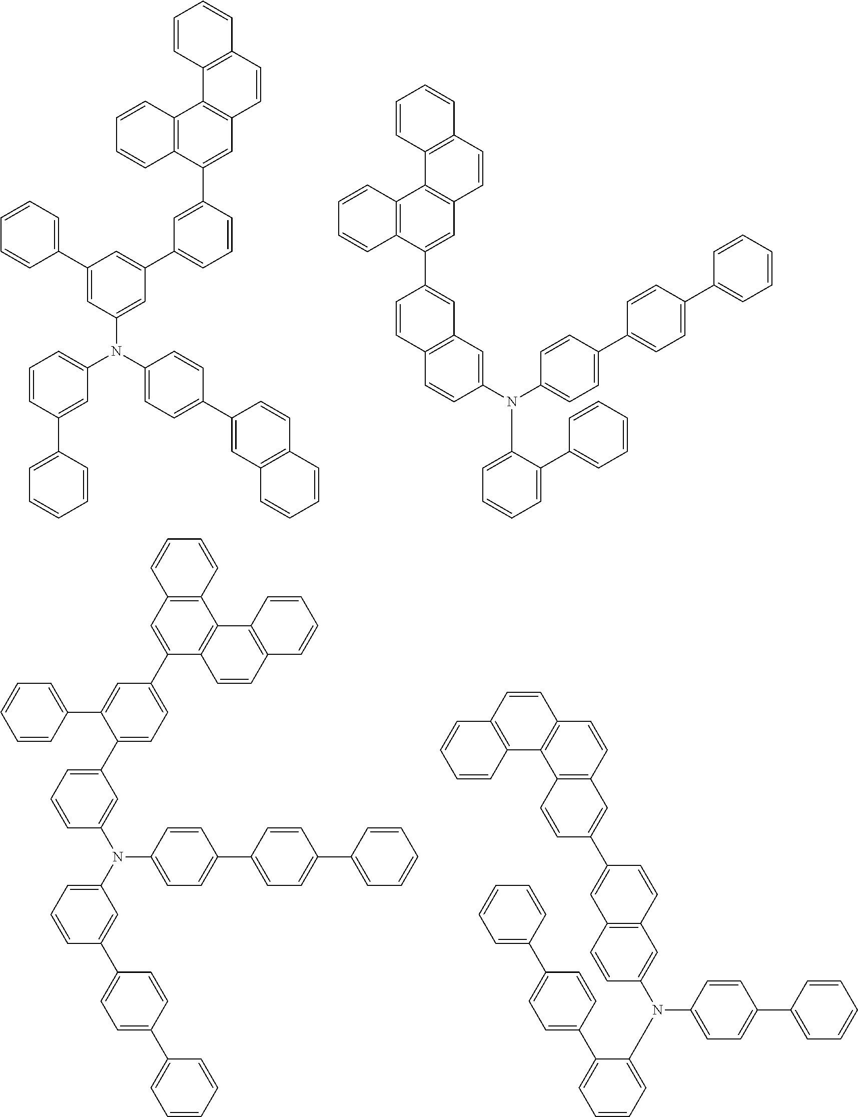 [SMILES: c1ccc(-c2cccc(N(c3ccc(-c4ccc5ccccc5c4)cc3)c3cc(-c4ccccc4)cc(-c4cccc(-c5cc6ccc7ccccc7c6c6ccccc56)c4)c3)c2)cc1 ; c1ccc(-c2ccc(-c3ccc(N(c4ccc5ccc(-c6cc7ccc8ccccc8c7c7ccccc67)cc5c4)c4ccccc4-c4ccccc4)cc3)cc2)cc1 ; c1ccc(-c2ccc(-c3ccc(N(c4cccc(-c5ccc(-c6ccccc6)cc5)c4)c4cccc(-c5ccc(-c6cc7ccccc7c7c6ccc6ccccc67)cc5-c5ccccc5)c4)cc3)cc2)cc1 ; c1ccc(-c2ccc(-c3ccccc3N(c3ccc(-c4ccccc4)cc3)c3ccc4cc(-c5ccc6c(ccc7ccc8ccccc8c76)c5)ccc4c3)cc2)cc1]

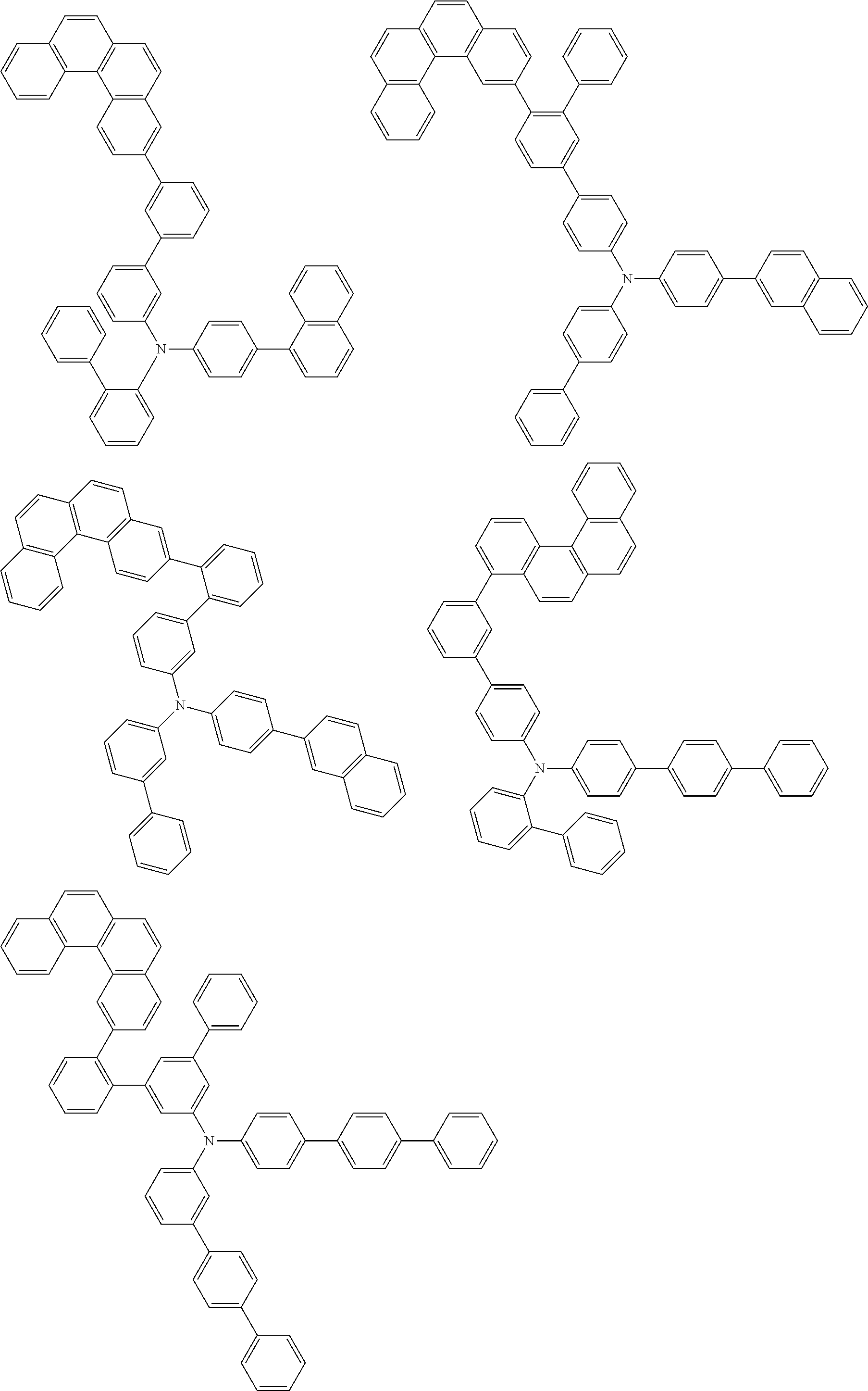

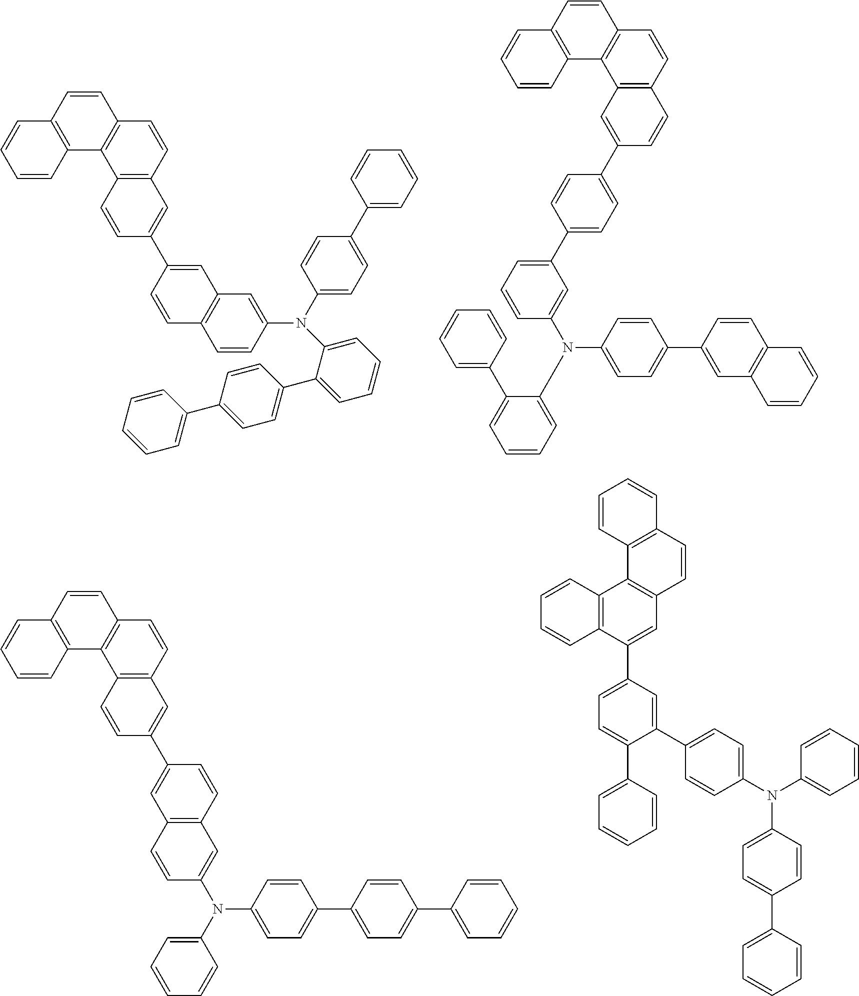 [SMILES: c1ccc(-c2ccc(-c3ccccc3N(c3ccc(-c4ccccc4)cc3)c3ccc4ccc(-c5ccc6c(ccc7ccc8ccccc8c76)c5)cc4c3)cc2)cc1 ; c1ccc(-c2ccccc2N(c2ccc(-c3ccc4ccccc4c3)cc2)c2cccc(-c3ccc(-c4ccc5ccc6ccc7ccccc7c6c5c4)cc3)c2)cc1 ; c1ccc(-c2ccc(-c3ccc(N(c4ccccc4)c4ccc5cc(-c6ccc7c(ccc8ccc9ccccc9c87)c6)ccc5c4)cc3)cc2)cc1 ; c1ccc(-c2ccc(N(c3ccccc3)c3ccc(-c4cc(-c5cc6ccc7ccccc7c6c6ccccc56)ccc4-c4ccccc4)cc3)cc2)cc1]

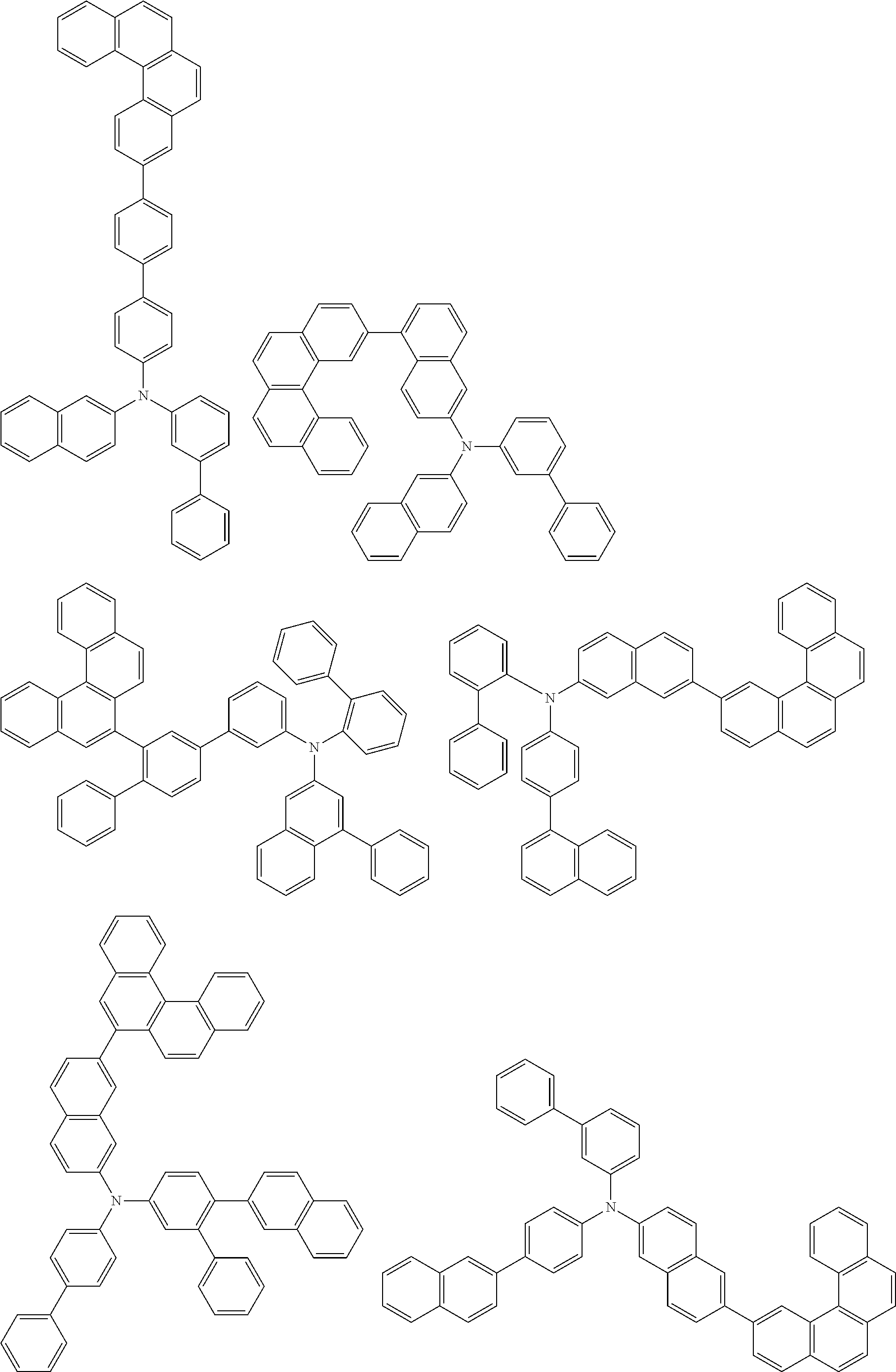

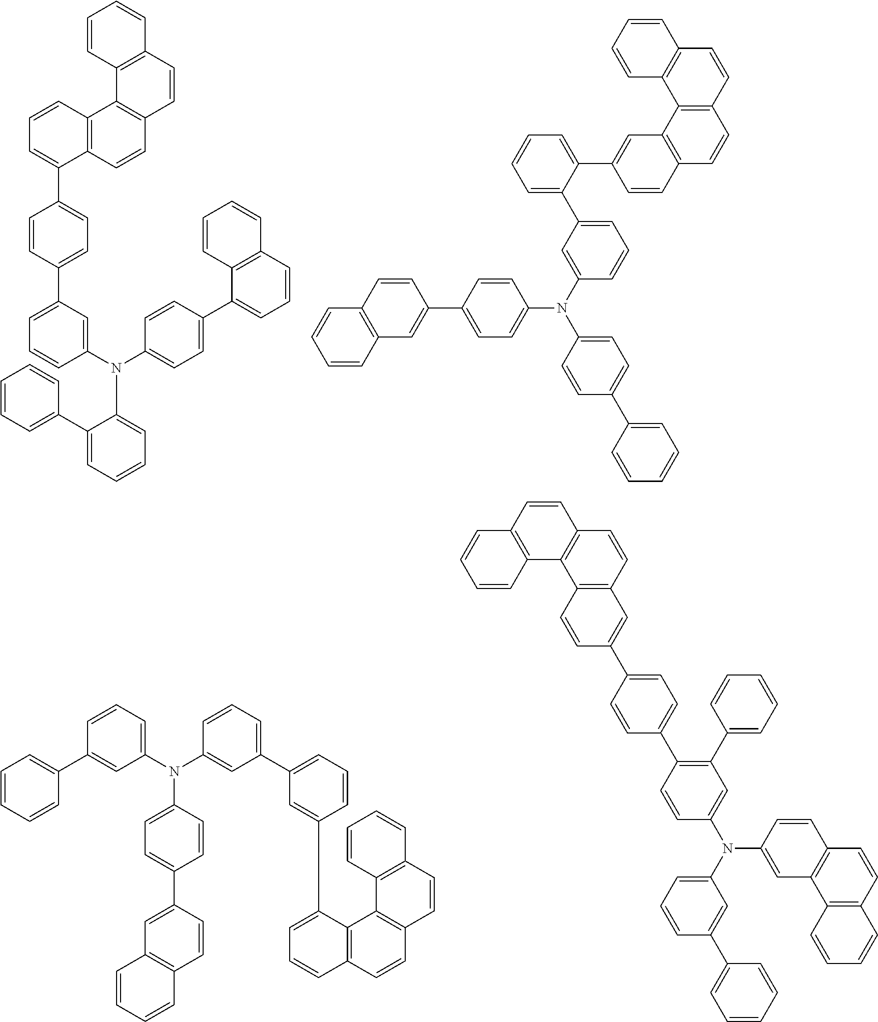

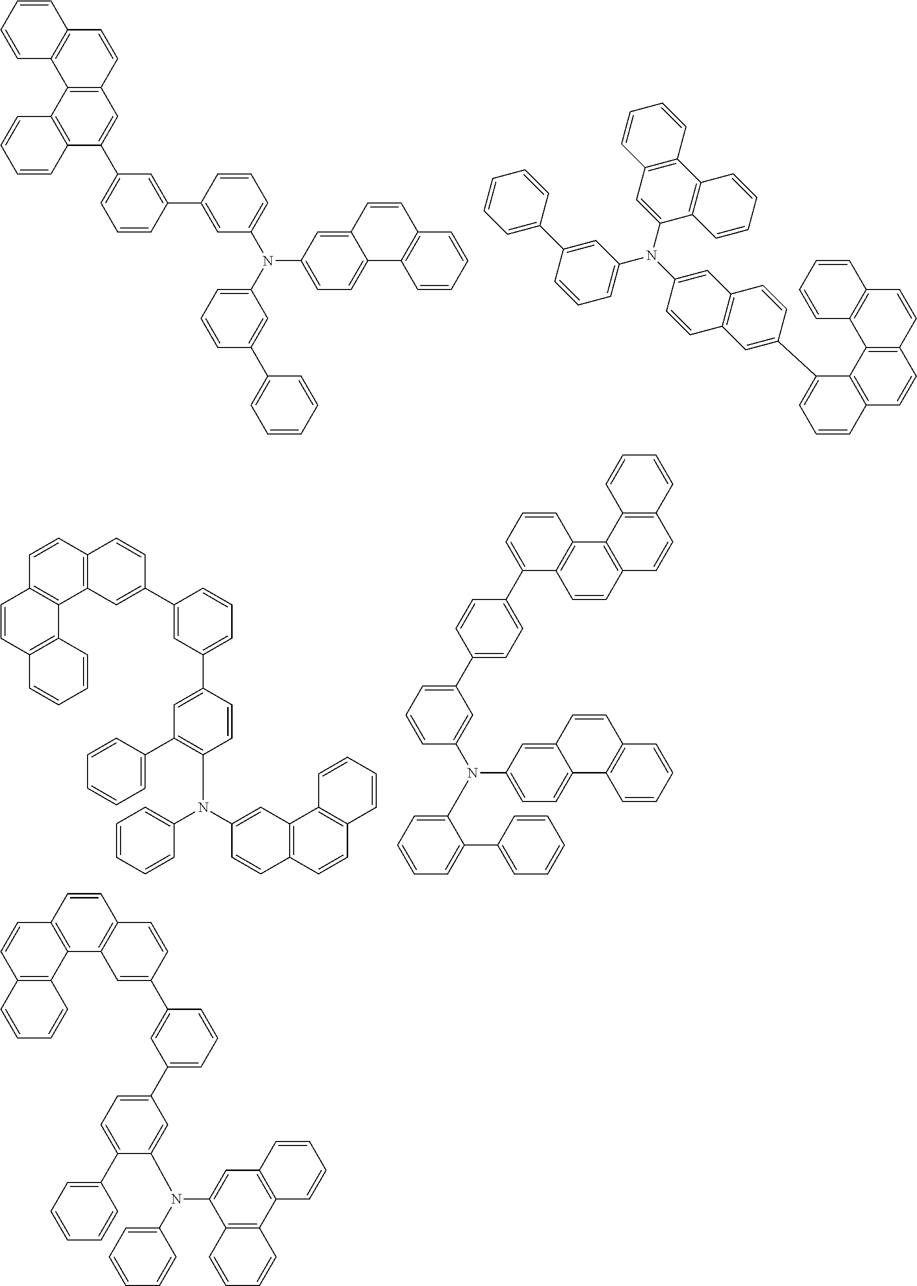

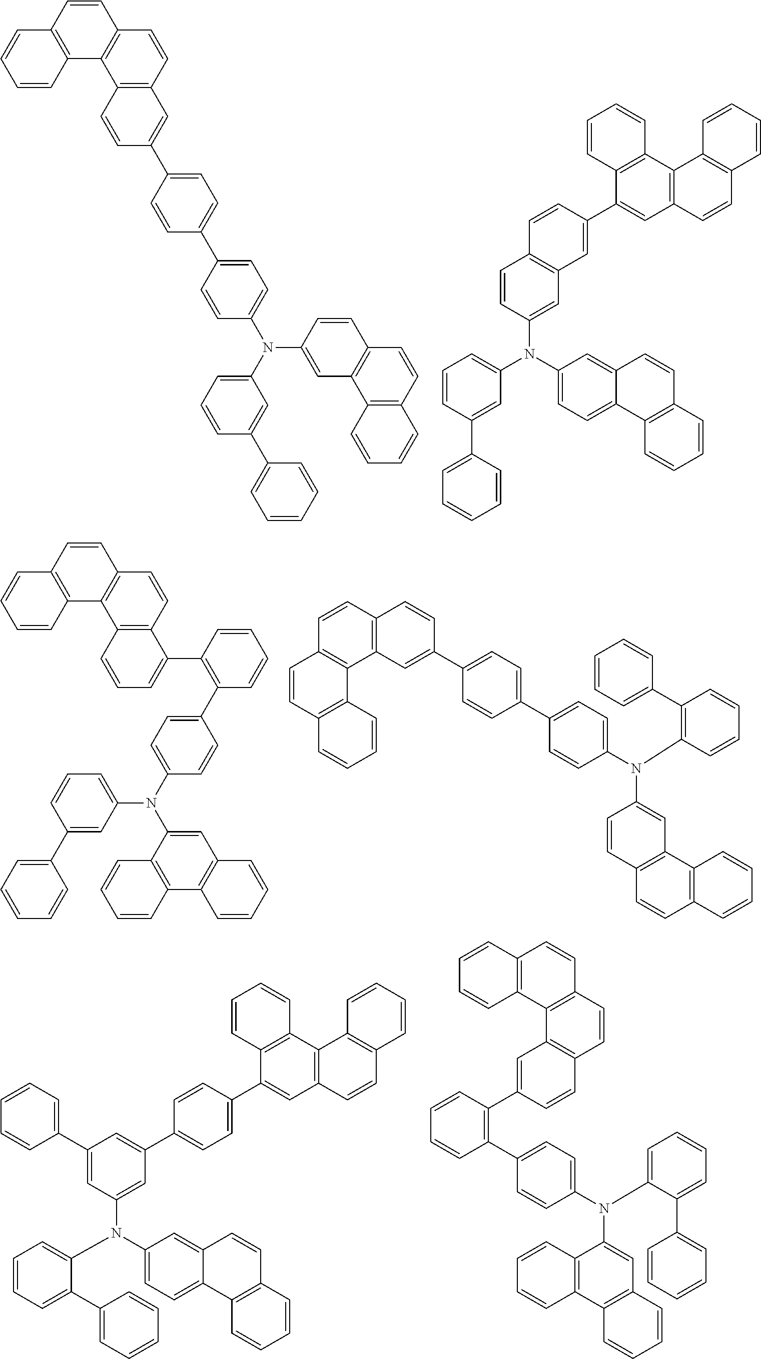

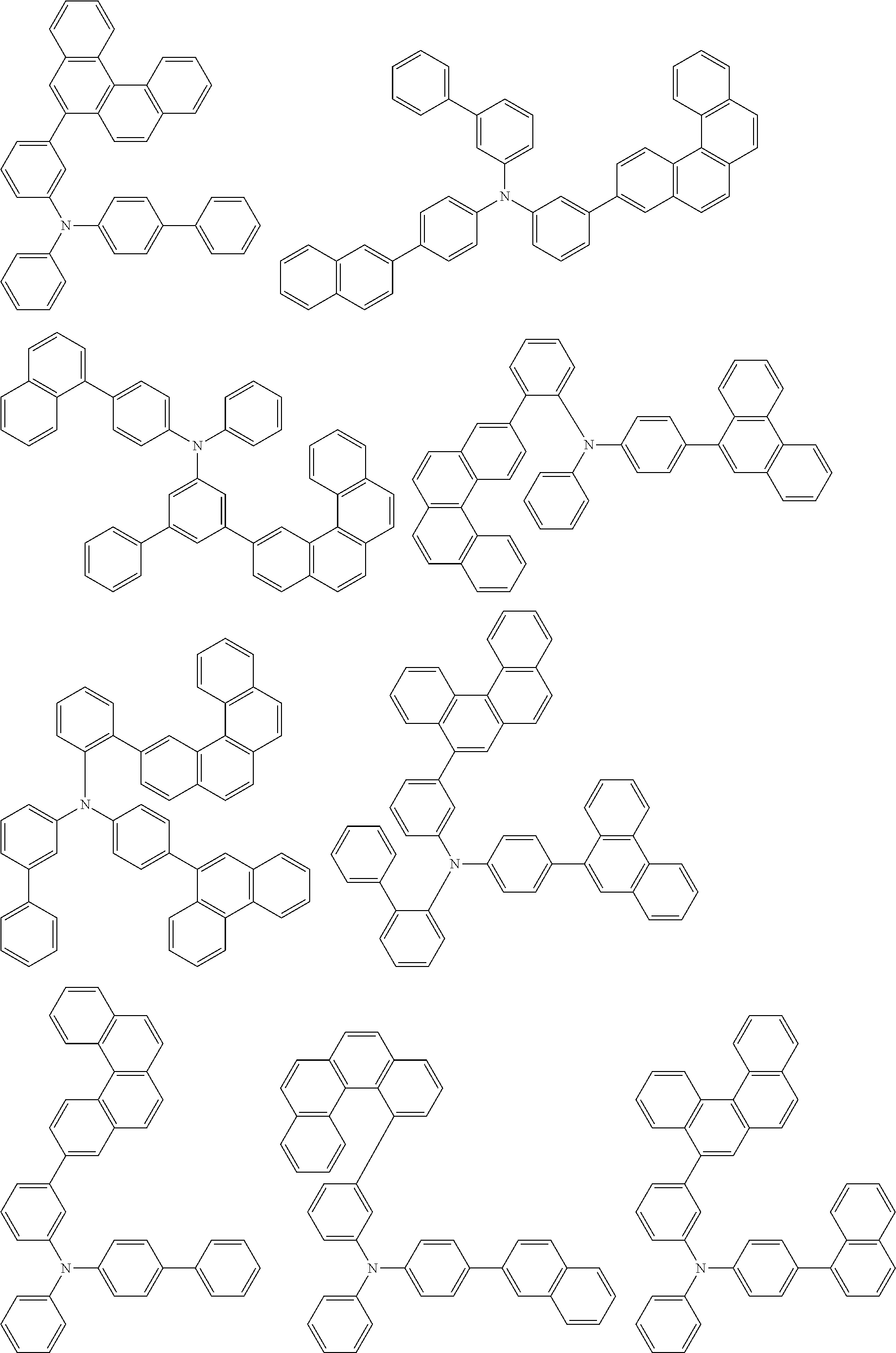

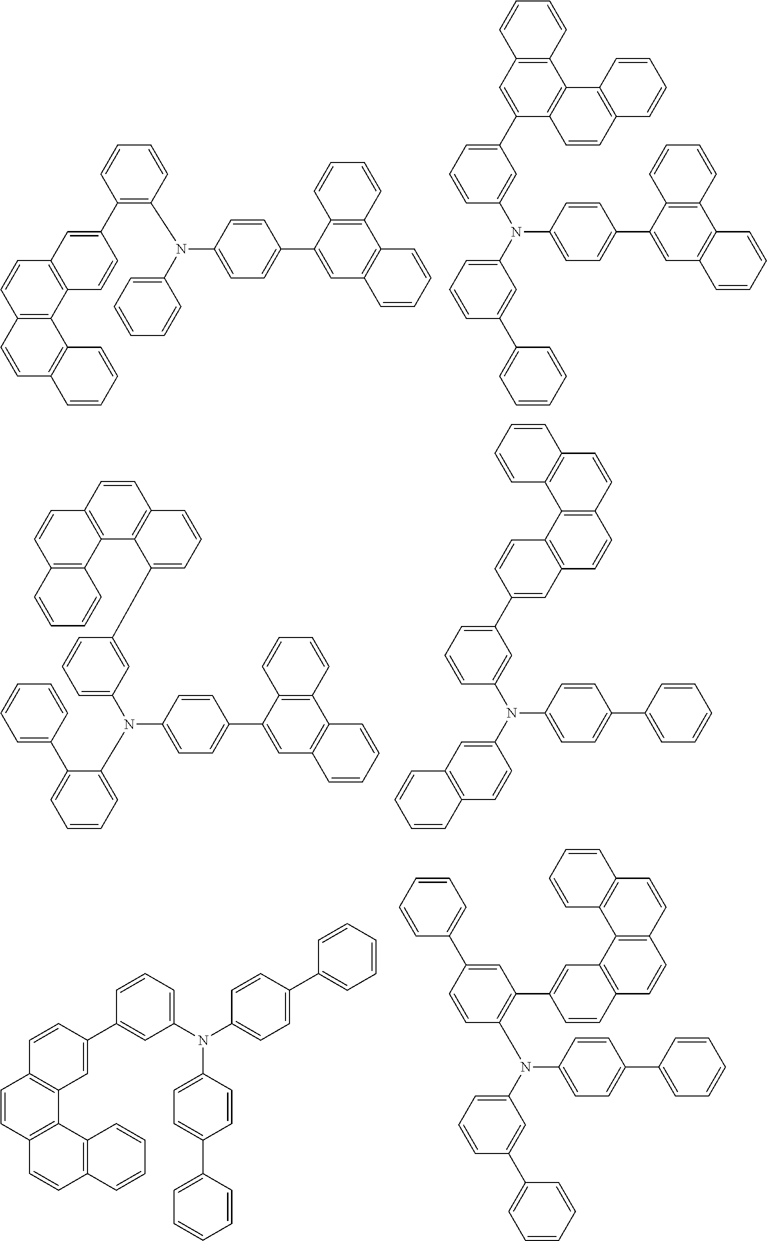

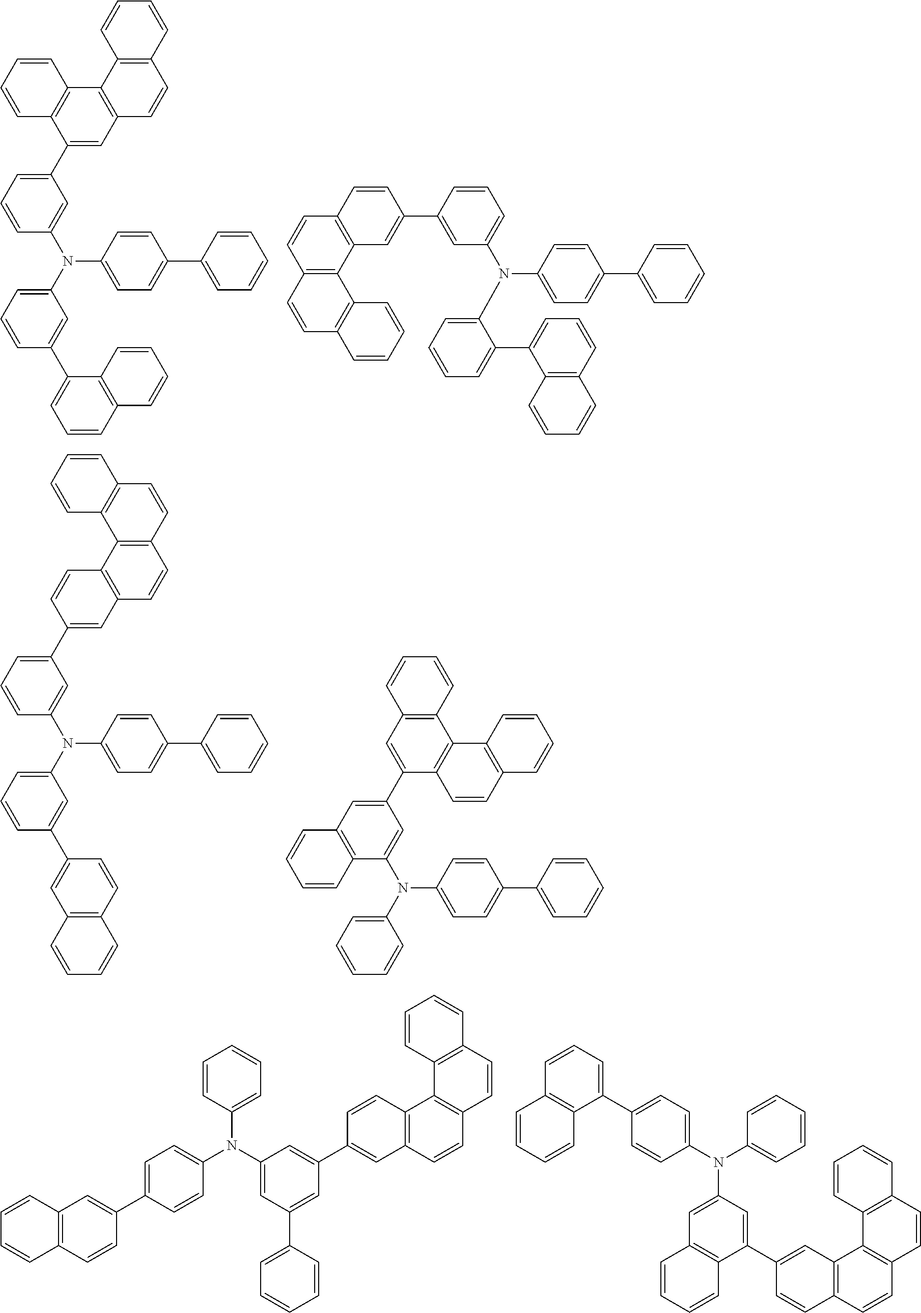

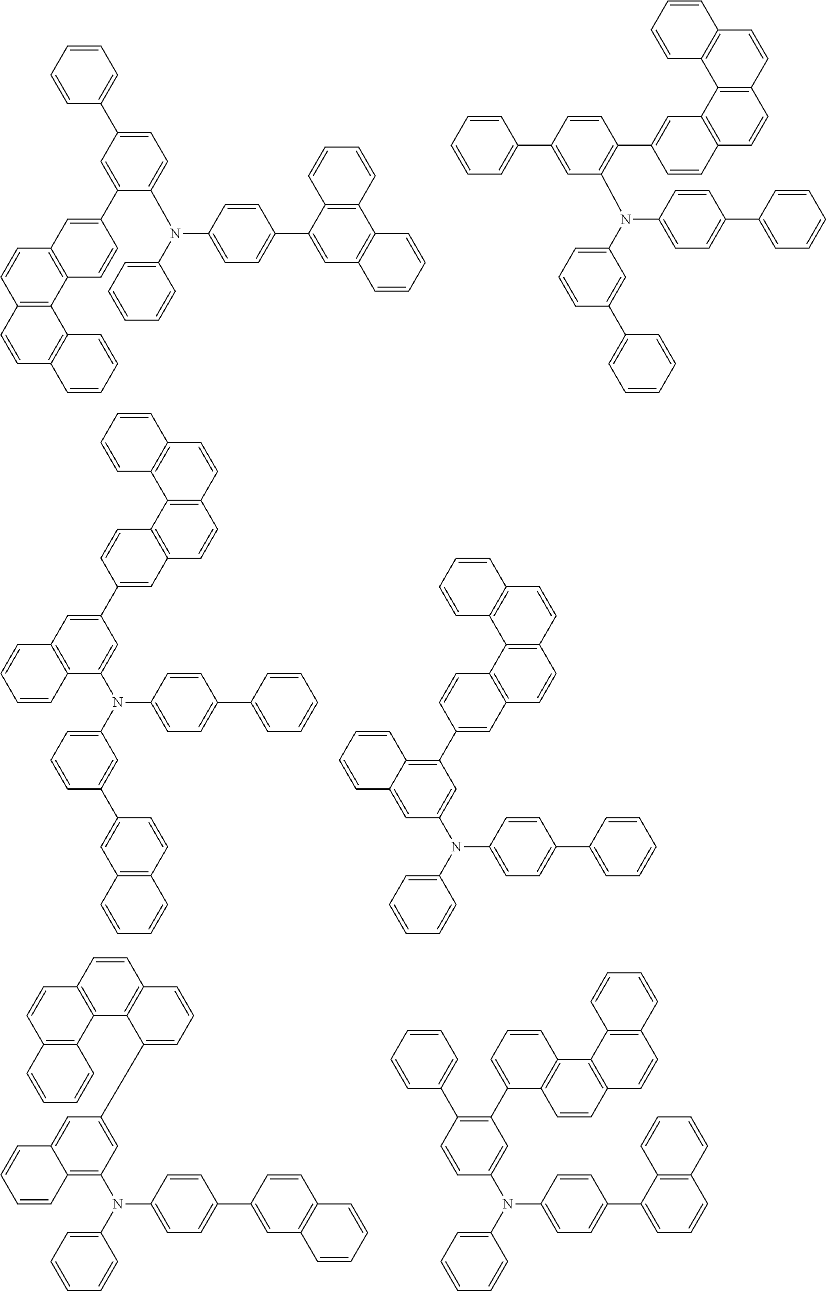

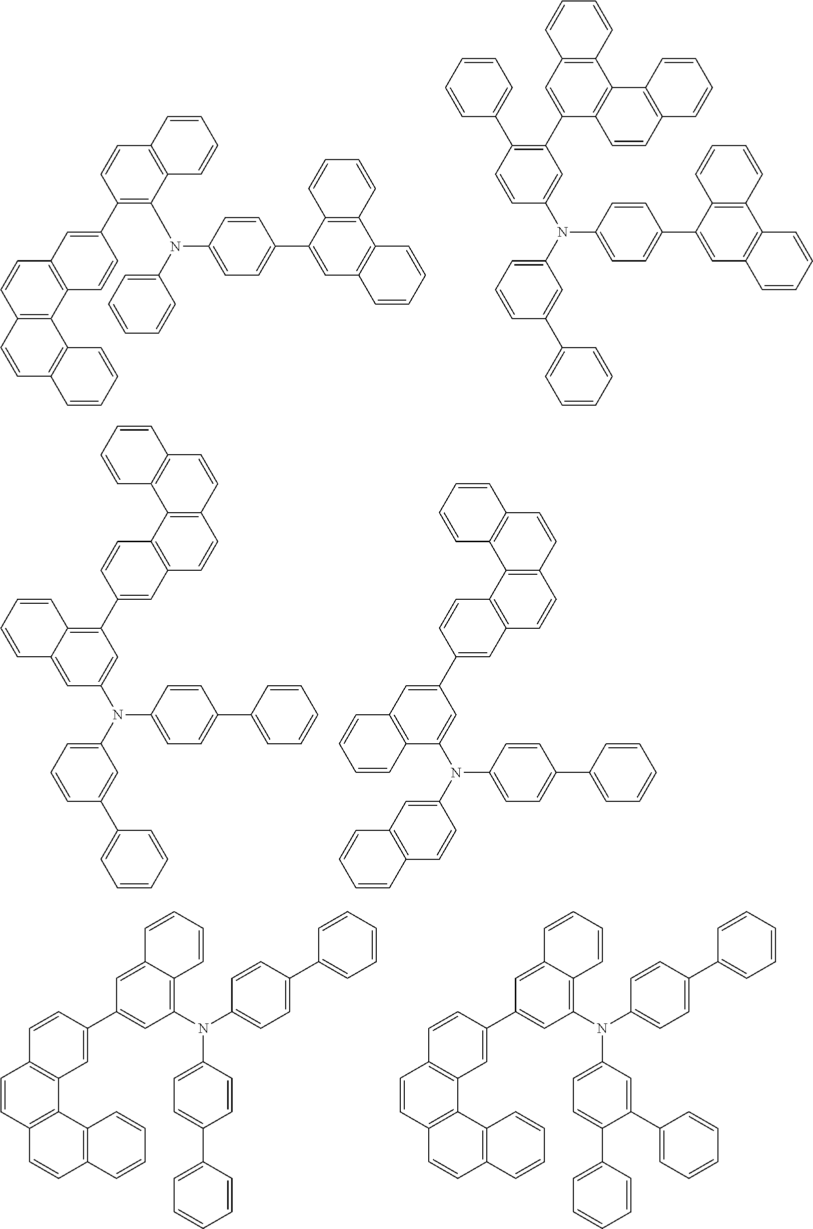

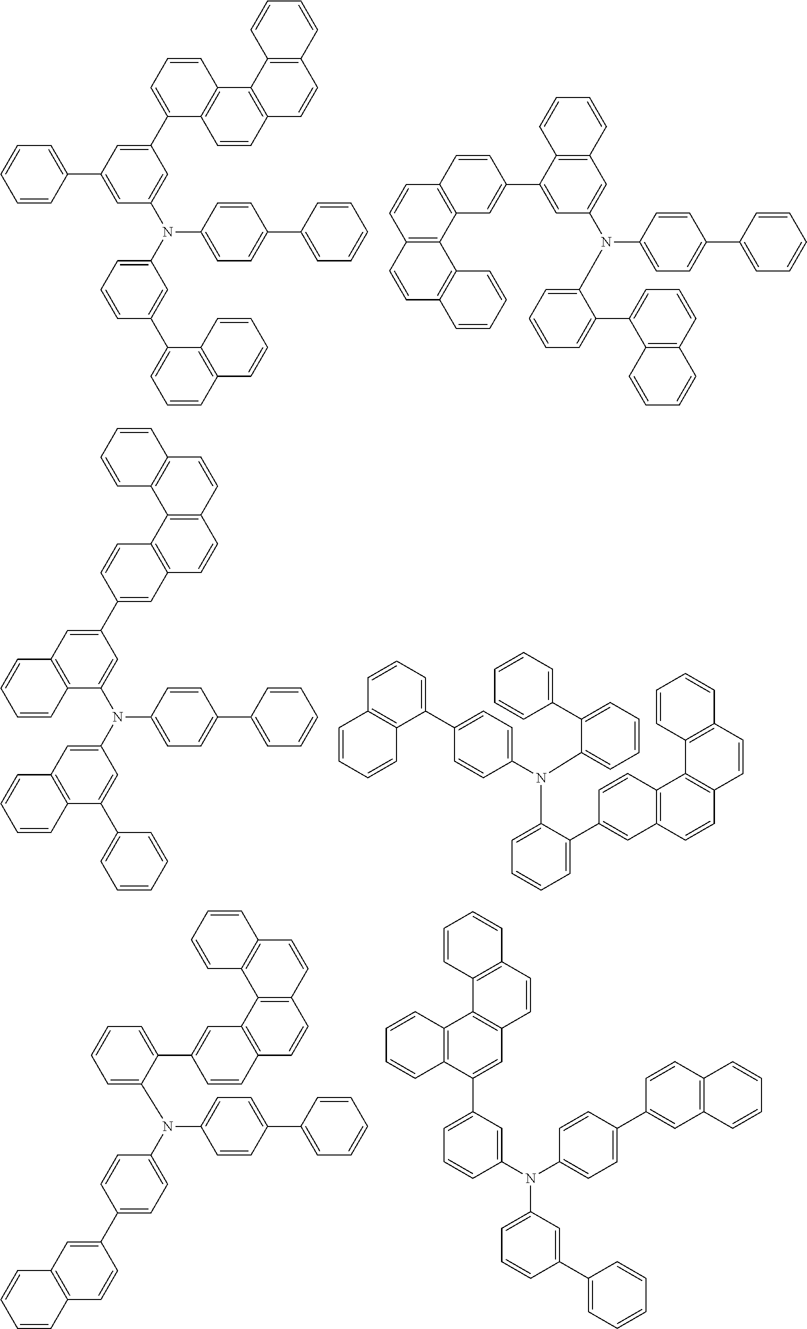

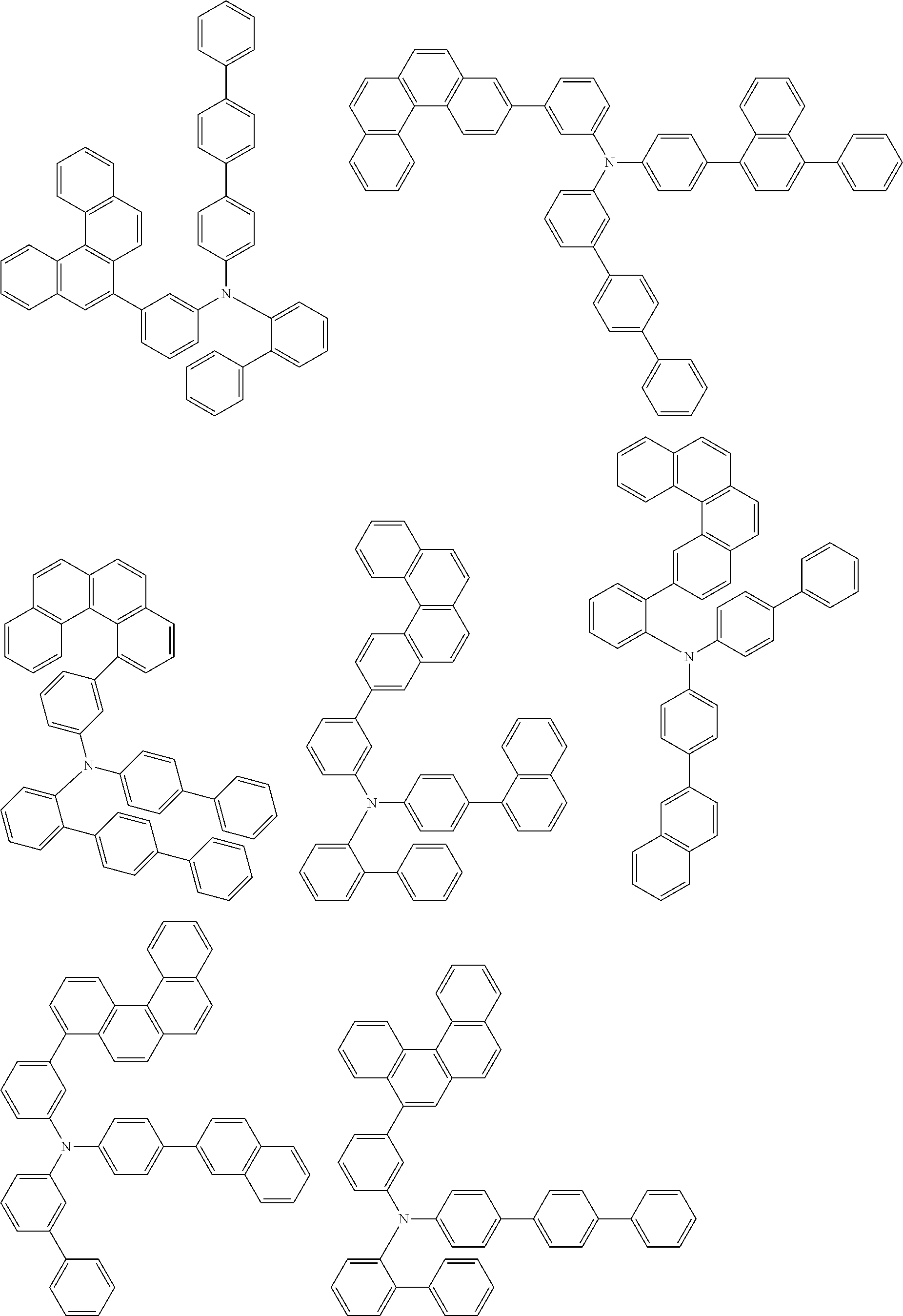

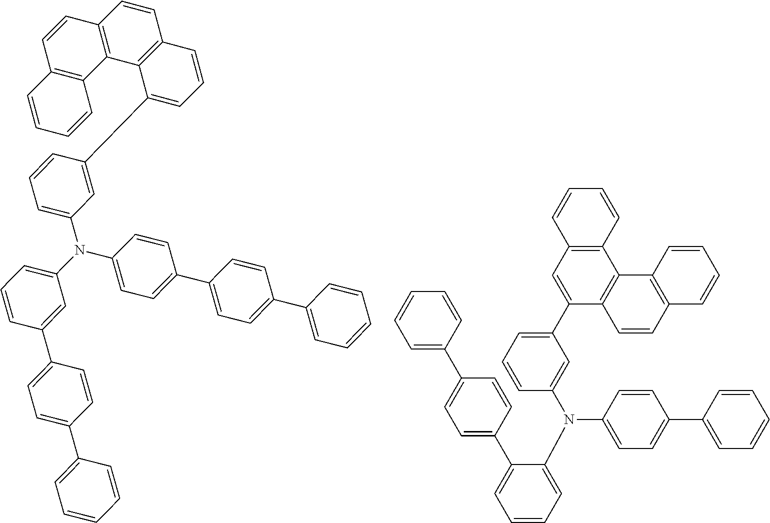

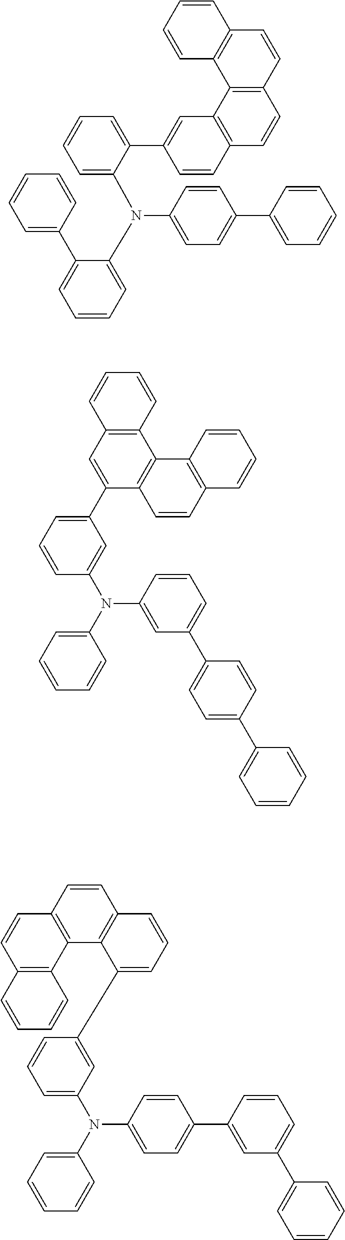 [SMILES: c1ccc(-c2ccc(N(c3ccccc3-c3ccccc3)c3ccccc3-c3ccc4ccc5ccc6ccccc6c5c4c3)cc2)cc1 ; c1ccc(-c2ccc(-c3cccc(N(c4ccccc4)c4cccc(-c5cc6ccccc6c6c5ccc5ccccc56)c4)c3)cc2)cc1 ; c1ccc(-c2cccc(-c3ccc(N(c4ccccc4)c4cccc(-c5cccc6ccc7ccc8ccccc8c7c56)c4)cc3)c2)cc1]

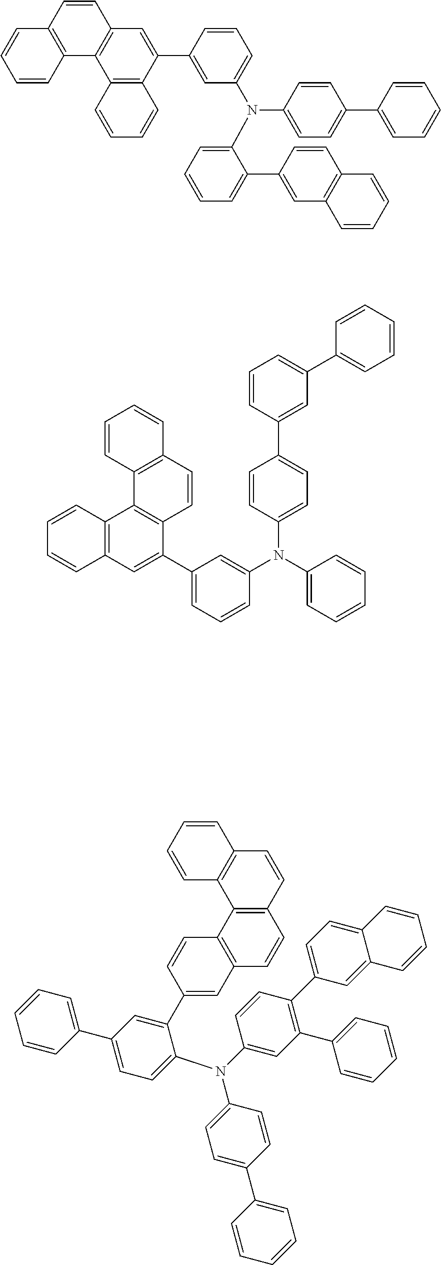 [SMILES: c1ccc(-c2ccc(N(c3cccc(-c4cc5ccc6ccccc6c5c5ccccc45)c3)c3ccccc3-c3ccc4ccccc4c3)cc2)cc1 ; c1ccc(-c2cccc(-c3ccc(N(c4ccccc4)c4cccc(-c5cc6ccccc6c6c5ccc5ccccc56)c4)cc3)c2)cc1 ; c1ccc(-c2ccc(N(c3ccc(-c4ccc5ccccc5c4)c(-c4ccccc4)c3)c3ccc(-c4ccccc4)cc3-c3ccc4c(ccc5ccc6ccccc6c54)c3)cc2)cc1]

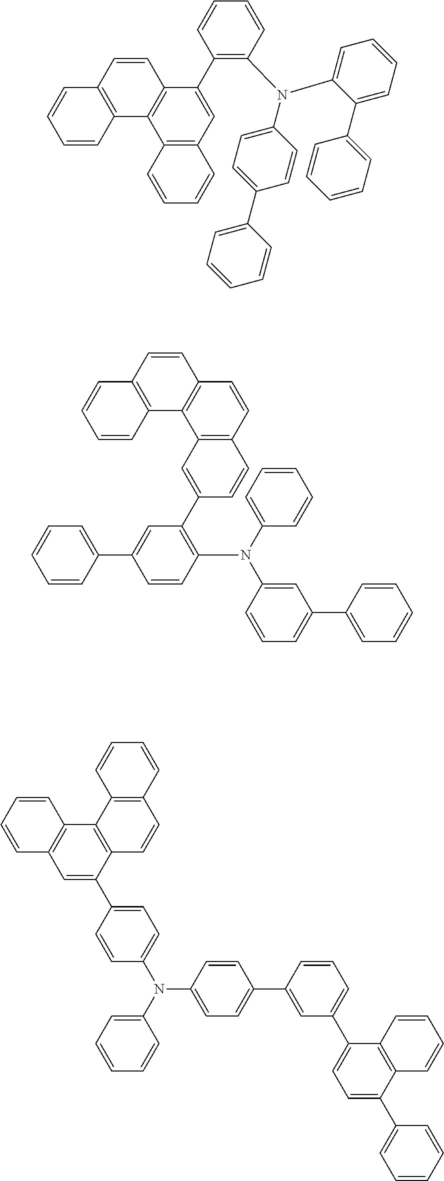 [SMILES: c1ccc(-c2ccc(N(c3ccccc3-c3ccccc3)c3ccccc3-c3cc4ccccc4c4c3ccc3ccccc34)cc2)cc1 ; c1ccc(-c2cccc(N(c3ccccc3)c3ccc(-c4ccccc4)cc3-c3ccc4ccc5ccc6ccccc6c5c4c3)c2)cc1 ; c1ccc(-c2ccc(-c3cccc(-c4ccc(N(c5ccccc5)c5ccc(-c6cc7ccccc7c7c6ccc6ccccc67)cc5)cc4)c3)c3ccccc23)cc1]

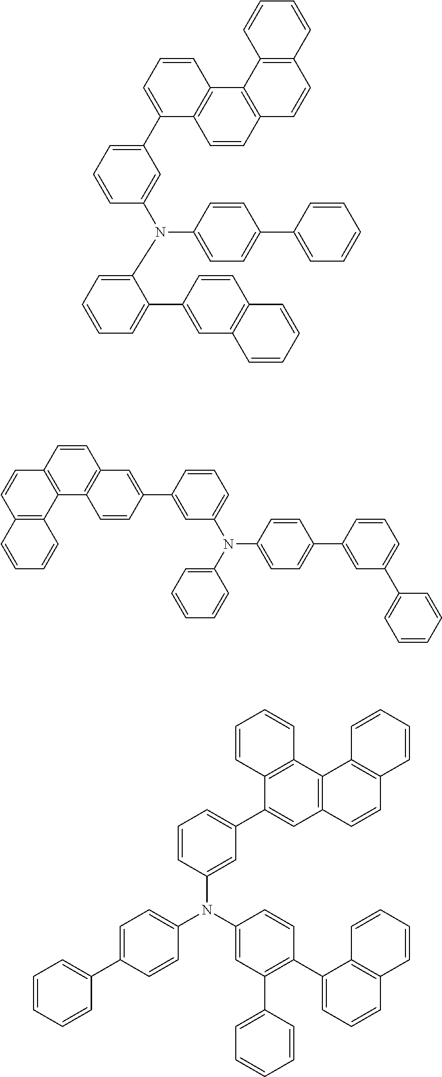 [SMILES: c1ccc(-c2ccc(N(c3cccc(-c4cccc5c4ccc4ccc6ccccc6c45)c3)c3ccccc3-c3ccc4ccccc4c3)cc2)cc1 ; c1ccc(-c2cccc(-c3ccc(N(c4ccccc4)c4cccc(-c5ccc6c(ccc7ccc8ccccc8c76)c5)c4)cc3)c2)cc1 ; c1ccc(-c2ccc(N(c3cccc(-c4cc5ccc6ccccc6c5c5ccccc45)c3)c3ccc(-c4cccc5ccccc45)c(-c4ccccc4)c3)cc2)cc1]

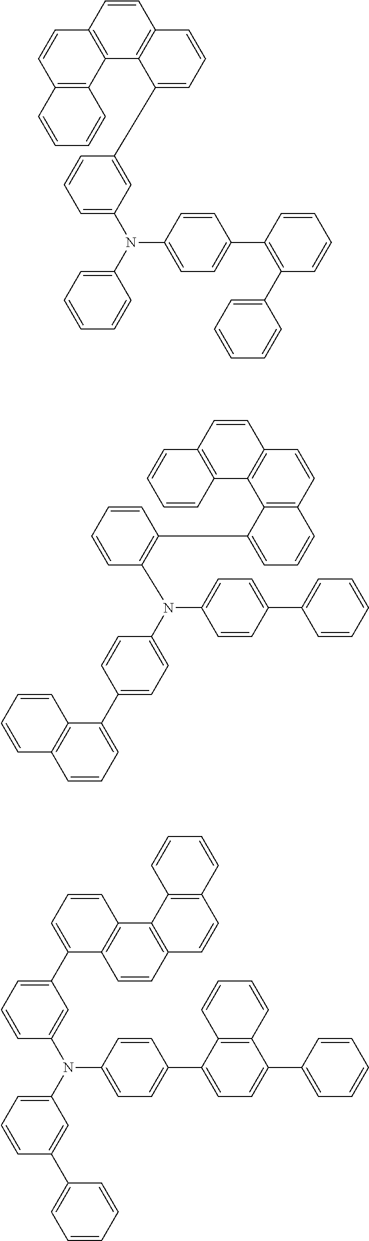 [SMILES: c1ccc(-c2ccccc2-c2ccc(N(c3ccccc3)c3cccc(-c4cccc5ccc6ccc7ccccc7c6c45)c3)cc2)cc1 ; c1ccc(-c2ccc(N(c3ccc(-c4cccc5ccccc45)cc3)c3ccccc3-c3cccc4ccc5ccc6ccccc6c5c34)cc2)cc1 ; c1ccc(-c2cccc(N(c3ccc(-c4ccc(-c5ccccc5)c5ccccc45)cc3)c3cccc(-c4cccc5c4ccc4ccc6ccccc6c45)c3)c2)cc1]

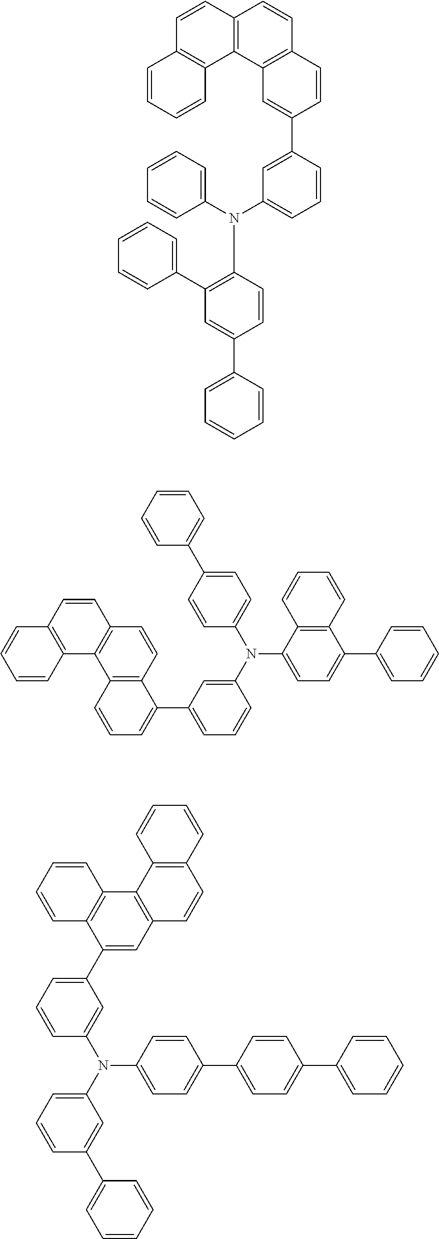 [SMILES: c1ccc(-c2ccc(N(c3ccccc3)c3cccc(-c4ccc5ccc6ccc7ccccc7c6c5c4)c3)c(-c3ccccc3)c2)cc1 ; c1ccc(-c2ccc(N(c3cccc(-c4cccc5c4ccc4ccc6ccccc6c45)c3)c3ccc(-c4ccccc4)c4ccccc34)cc2)cc1 ; c1ccc(-c2ccc(-c3ccc(N(c4cccc(-c5ccccc5)c4)c4cccc(-c5cc6ccc7ccccc7c6c6ccccc56)c4)cc3)cc2)cc1]

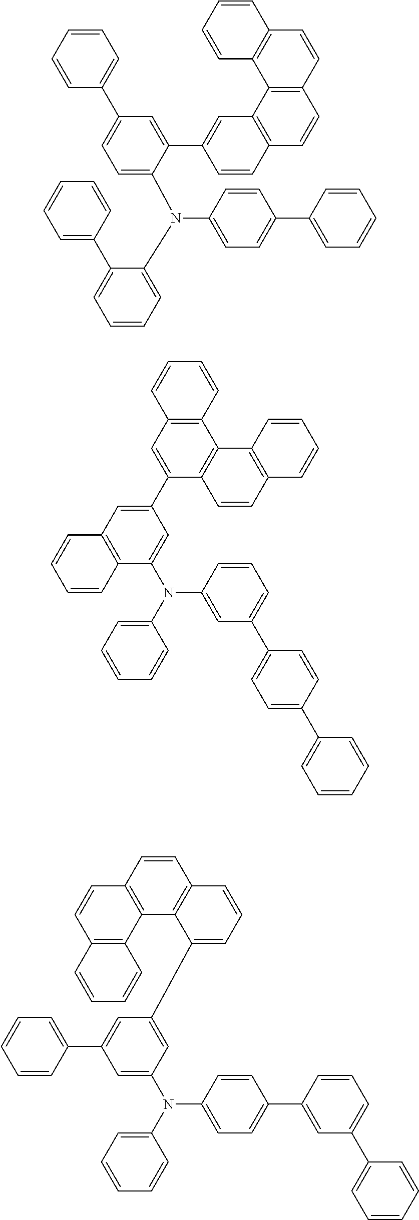

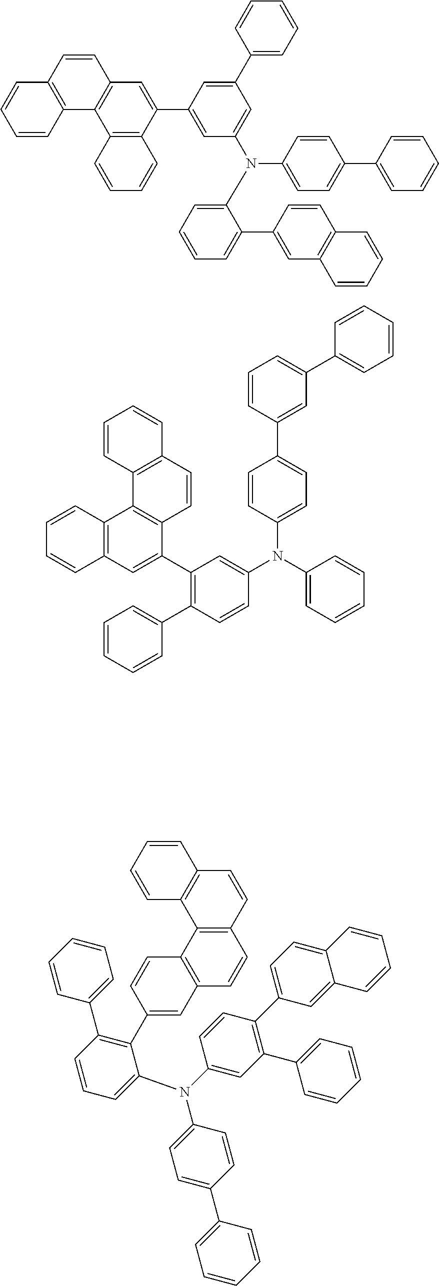

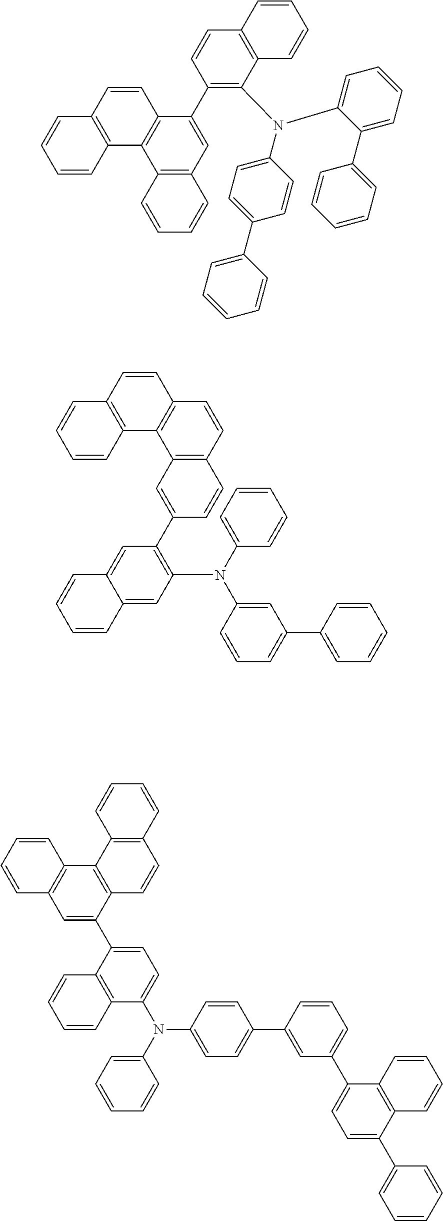 [SMILES: c1ccc(-c2ccc(N(c3ccccc3-c3ccccc3)c3c(-c4cc5ccccc5c5c4ccc4ccccc45)ccc4ccccc34)cc2)cc1 ; c1ccc(-c2cccc(N(c3ccccc3)c3cc4ccccc4cc3-c3ccc4ccc5ccc6ccccc6c5c4c3)c2)cc1 ; c1ccc(-c2ccc(-c3cccc(-c4ccc(N(c5ccccc5)c5ccc(-c6cc7ccccc7c7c6ccc6ccccc67)c6ccccc56)cc4)c3)c3ccccc23)cc1]

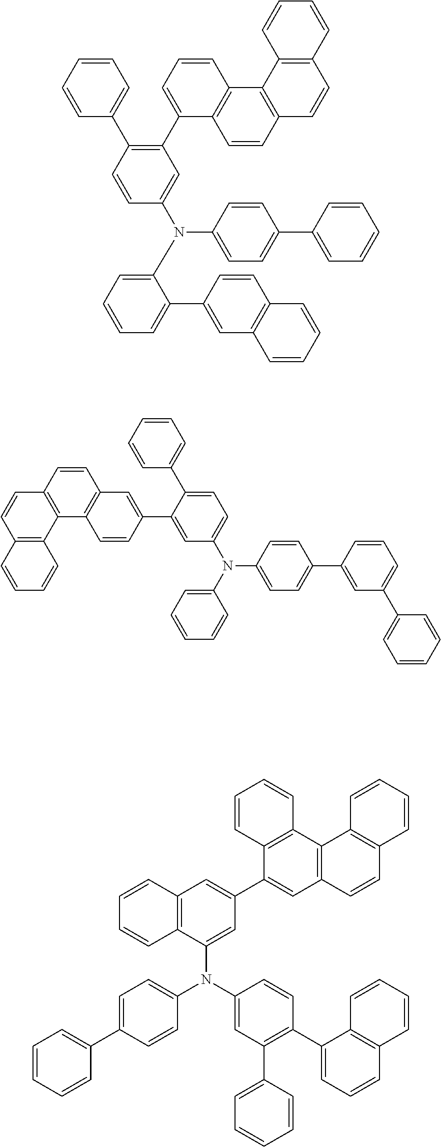 [SMILES: c1ccc(-c2ccc(N(c3ccc(-c4ccccc4)c(-c4cccc5c4ccc4ccc6ccccc6c45)c3)c3ccccc3-c3ccc4ccccc4c3)cc2)cc1 ; c1ccc(-c2cccc(-c3ccc(N(c4ccccc4)c4ccc(-c5ccccc5)c(-c5ccc6c(ccc7ccc8ccccc8c76)c5)c4)cc3)c2)cc1 ; c1ccc(-c2ccc(N(c3ccc(-c4cccc5ccccc45)c(-c4ccccc4)c3)c3cc(-c4cc5ccc6ccccc6c5c5ccccc45)cc4ccccc34)cc2)cc1]

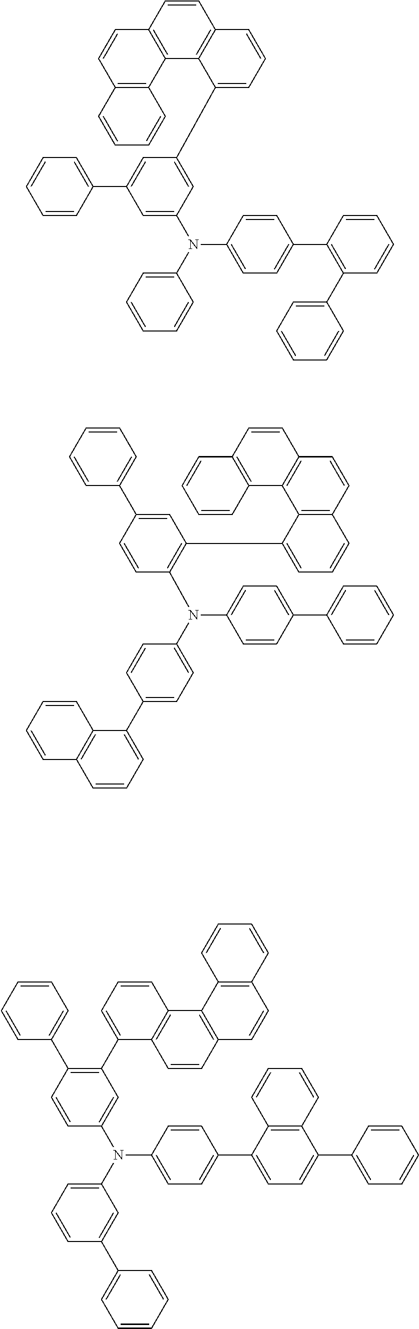 [SMILES: c1ccc(-c2cc(-c3cccc4ccc5ccc6ccccc6c5c34)cc(N(c3ccccc3)c3ccc(-c4ccccc4-c4ccccc4)cc3)c2)cc1 ; c1ccc(-c2ccc(N(c3ccc(-c4cccc5ccccc45)cc3)c3ccc(-c4ccccc4)cc3-c3cccc4ccc5ccc6ccccc6c5c34)cc2)cc1 ; c1ccc(-c2cccc(N(c3ccc(-c4ccc(-c5ccccc5)c5ccccc45)cc3)c3ccc(-c4ccccc4)c(-c4cccc5c4ccc4ccc6ccccc6c45)c3)c2)cc1]

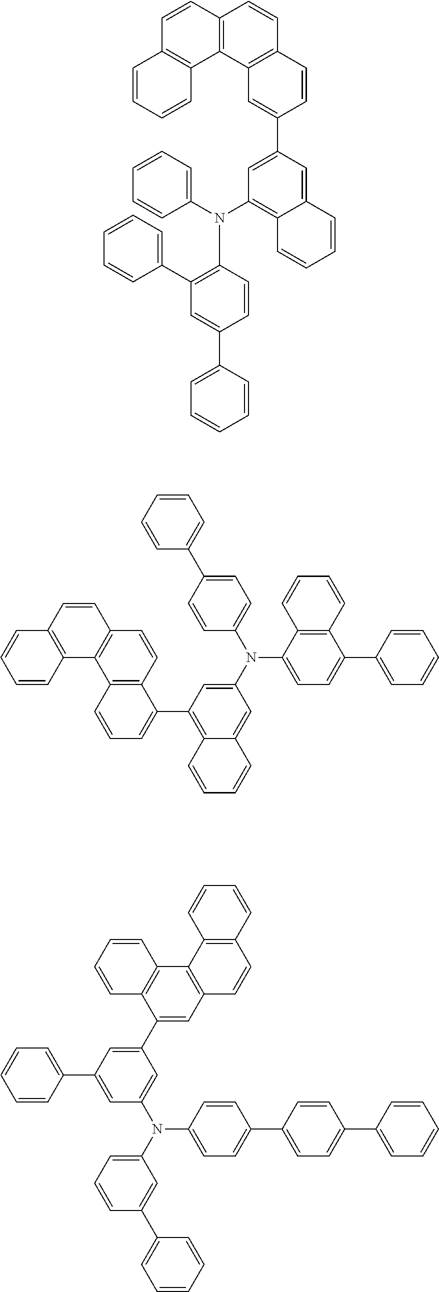 [SMILES: c1ccc(-c2ccc(N(c3ccccc3)c3cc(-c4ccc5ccc6ccc7ccccc7c6c5c4)cc4ccccc34)c(-c3ccccc3)c2)cc1 ; c1ccc(-c2ccc(N(c3cc(-c4cccc5c4ccc4ccc6ccccc6c45)c4ccccc4c3)c3ccc(-c4ccccc4)c4ccccc34)cc2)cc1 ; c1ccc(-c2ccc(-c3ccc(N(c4cccc(-c5ccccc5)c4)c4cc(-c5ccccc5)cc(-c5cc6ccc7ccccc7c6c6ccccc56)c4)cc3)cc2)cc1]

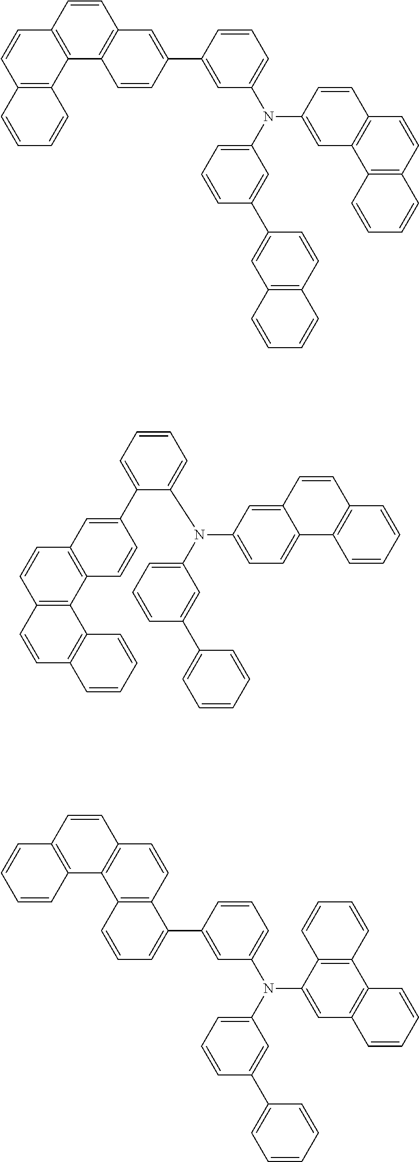 [SMILES: c1cc(-c2ccc3ccccc3c2)cc(N(c2cccc(-c3ccc4c(ccc5ccc6ccccc6c54)c3)c2)c2ccc3ccc4ccccc4c3c2)c1 ; c1ccc(-c2cccc(N(c3ccc4c(ccc5ccccc54)c3)c3ccccc3-c3ccc4c(ccc5ccc6ccccc6c54)c3)c2)cc1 ; c1ccc(-c2cccc(N(c3cccc(-c4cccc5c4ccc4ccc6ccccc6c45)c3)c3cc4ccccc4c4ccccc34)c2)cc1]

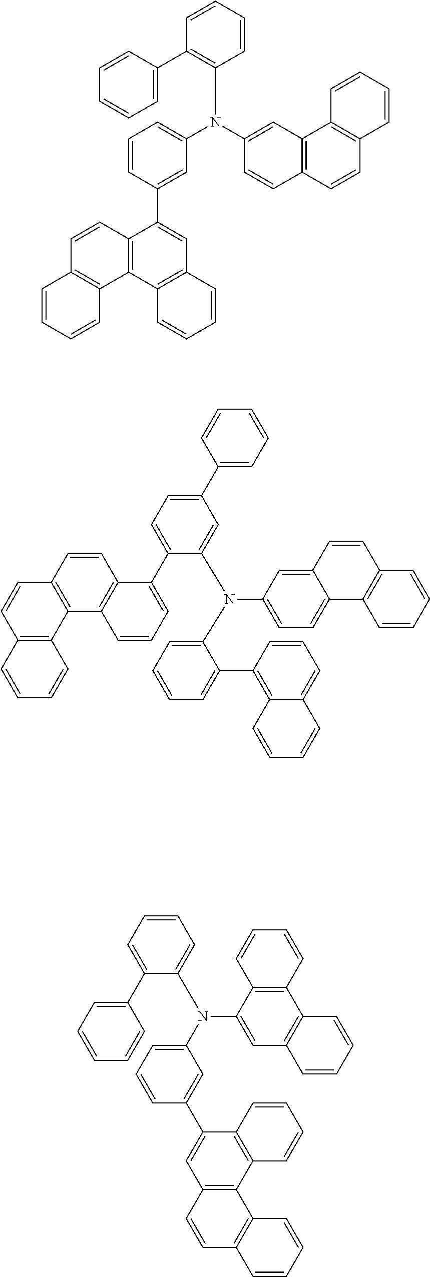 [SMILES: c1ccc(-c2ccccc2N(c2cccc(-c3cc4ccccc4c4c3ccc3ccccc34)c2)c2ccc3ccc4ccccc4c3c2)cc1 ; c1ccc(-c2ccc(-c3cccc4c3ccc3ccc5ccccc5c34)c(N(c3ccc4c(ccc5ccccc54)c3)c3ccccc3-c3cccc4ccccc34)c2)cc1 ; c1ccc(-c2ccccc2N(c2cccc(-c3cc4ccc5ccccc5c4c4ccccc34)c2)c2cc3ccccc3c3ccccc23)cc1]

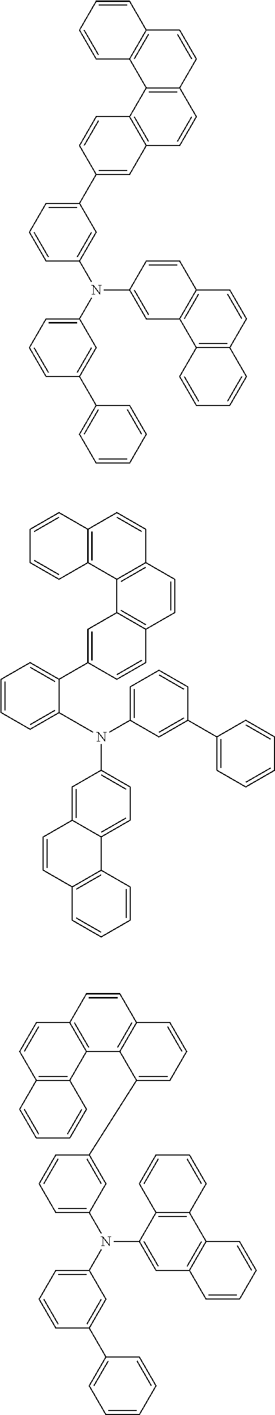 [SMILES: c1ccc(-c2cccc(N(c3cccc(-c4ccc5c(ccc6ccc7ccccc7c65)c4)c3)c3ccc4ccc5ccccc5c4c3)c2)cc1 ; c1ccc(-c2cccc(N(c3ccc4c(ccc5ccccc54)c3)c3ccccc3-c3ccc4ccc5ccc6ccccc6c5c4c3)c2)cc1 ; c1ccc(-c2cccc(N(c3cccc(-c4cccc5ccc6ccc7ccccc7c6c45)c3)c3cc4ccccc4c4ccccc34)c2)cc1]

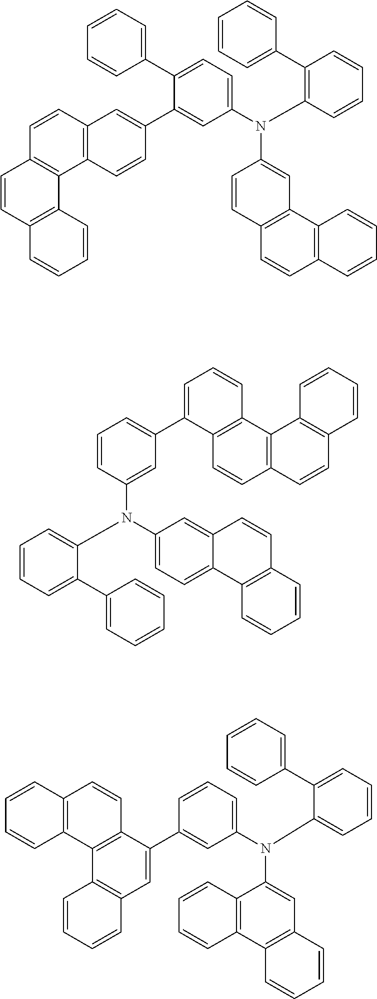 [SMILES: c1ccc(-c2ccc(N(c3ccc4ccc5ccccc5c4c3)c3ccccc3-c3ccccc3)cc2-c2ccc3c(ccc4ccc5ccccc5c43)c2)cc1 ; c1ccc(-c2ccccc2N(c2cccc(-c3cccc4c3ccc3ccc5ccccc5c34)c2)c2ccc3c(ccc4ccccc43)c2)cc1 ; c1ccc(-c2ccccc2N(c2cccc(-c3cc4ccccc4c4c3ccc3ccccc34)c2)c2cc3ccccc3c3ccccc23)cc1]

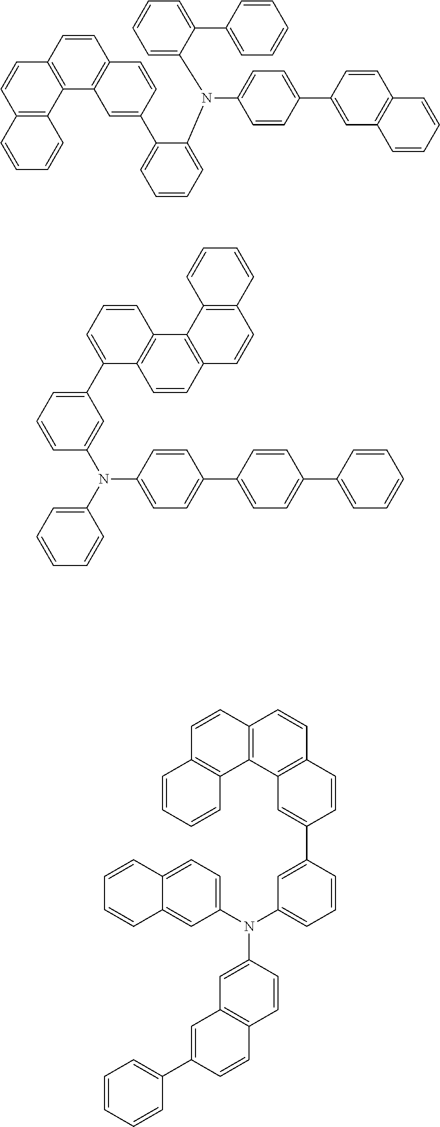 [SMILES: c1ccc(-c2ccccc2N(c2ccc(-c3ccc4ccccc4c3)cc2)c2ccccc2-c2ccc3ccc4ccc5ccccc5c4c3c2)cc1 ; c1ccc(-c2ccc(-c3ccc(N(c4ccccc4)c4cccc(-c5cccc6c5ccc5ccc7ccccc7c56)c4)cc3)cc2)cc1 ; c1ccc(-c2ccc3ccc(N(c4cccc(-c5ccc6ccc7ccc8ccccc8c7c6c5)c4)c4ccc5ccccc5c4)cc3c2)cc1]

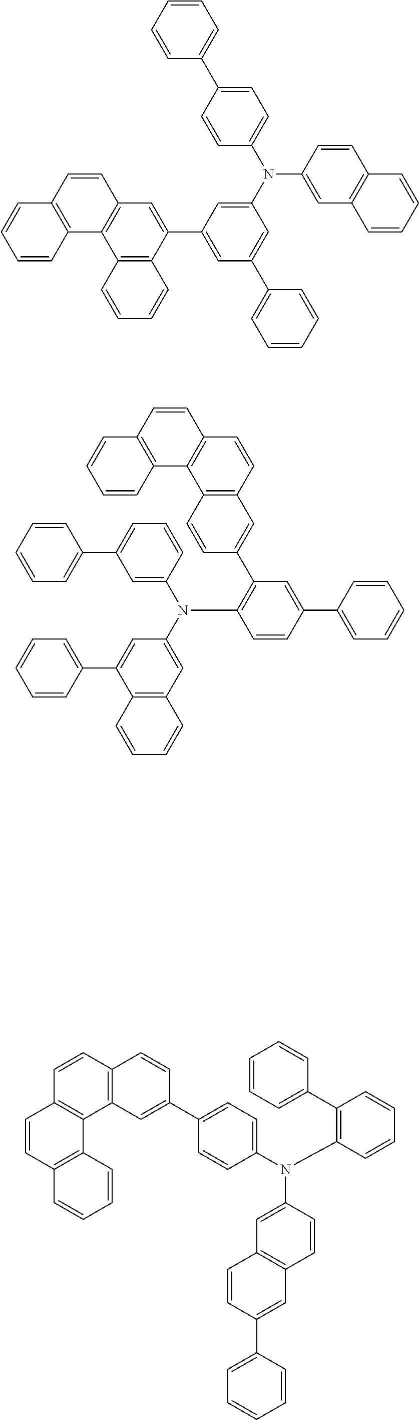 [SMILES: c1ccc(-c2ccc(N(c3cc(-c4ccccc4)cc(-c4cc5ccc6ccccc6c5c5ccccc45)c3)c3ccc4ccccc4c3)cc2)cc1 ; c1ccc(-c2cccc(N(c3cc(-c4ccccc4)c4ccccc4c3)c3ccc(-c4ccccc4)cc3-c3ccc4c(ccc5ccc6ccccc6c54)c3)c2)cc1 ; c1ccc(-c2ccc3cc(N(c4ccc(-c5ccc6ccc7ccc8ccccc8c7c6c5)cc4)c4ccccc4-c4ccccc4)ccc3c2)cc1]

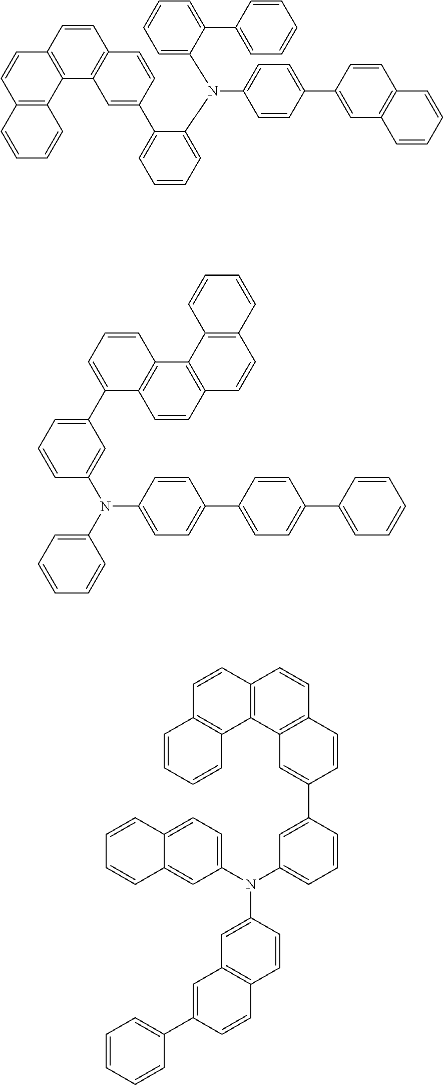 [SMILES: c1ccc(-c2ccccc2N(c2ccc(-c3ccc4ccccc4c3)cc2)c2ccccc2-c2ccc3ccc4ccc5ccccc5c4c3c2)cc1 ; c1ccc(-c2ccc(-c3ccc(N(c4ccccc4)c4cccc(-c5cccc6c5ccc5ccc7ccccc7c56)c4)cc3)cc2)cc1 ; c1ccc(-c2ccc3ccc(N(c4cccc(-c5ccc6ccc7ccc8ccccc8c7c6c5)c4)c4ccc5ccccc5c4)cc3c2)cc1]

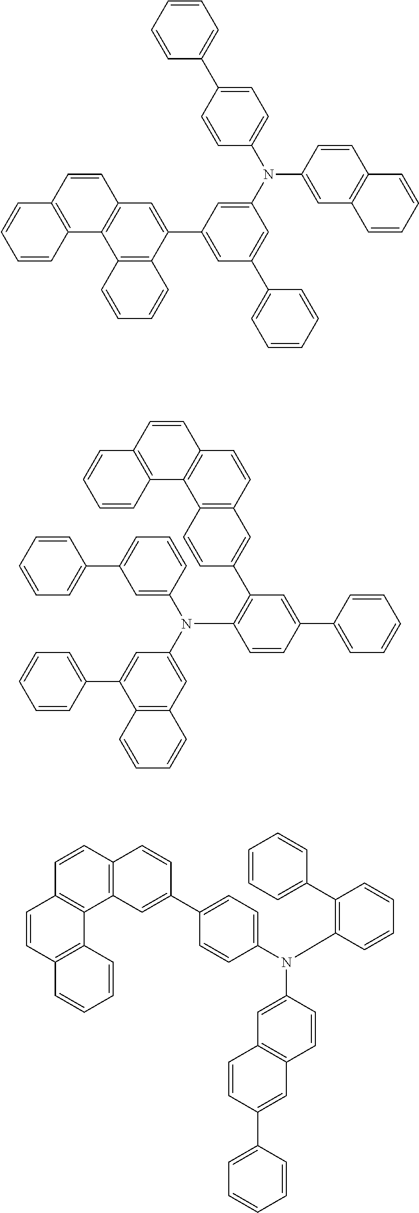 [SMILES: c1ccc(-c2ccc(N(c3cc(-c4ccccc4)cc(-c4cc5ccc6ccccc6c5c5ccccc45)c3)c3ccc4ccccc4c3)cc2)cc1 ; c1ccc(-c2cccc(N(c3cc(-c4ccccc4)c4ccccc4c3)c3ccc(-c4ccccc4)cc3-c3ccc4c(ccc5ccc6ccccc6c54)c3)c2)cc1 ; c1ccc(-c2ccc3cc(N(c4ccc(-c5ccc6ccc7ccc8ccccc8c7c6c5)cc4)c4ccccc4-c4ccccc4)ccc3c2)cc1]

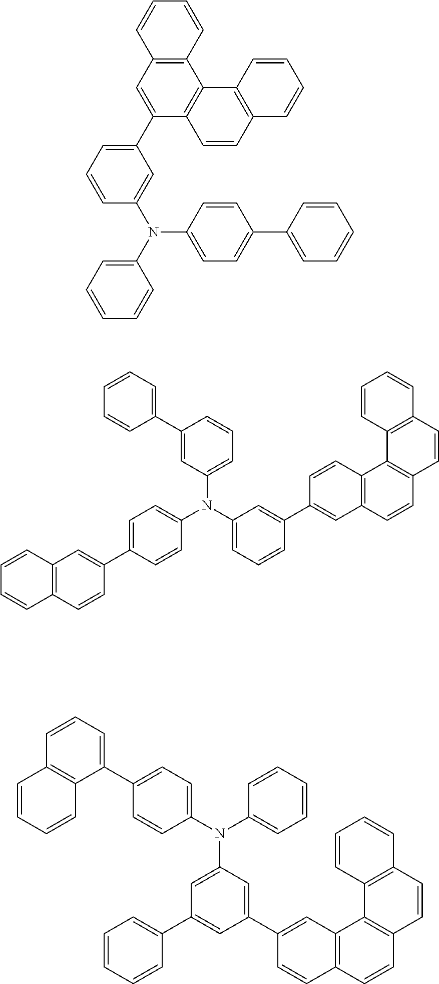 [SMILES: c1ccc(-c2ccc(N(c3ccccc3)c3cccc(-c4cc5ccccc5c5c4ccc4ccccc45)c3)cc2)cc1 ; c1ccc(-c2cccc(N(c3ccc(-c4ccc5ccccc5c4)cc3)c3cccc(-c4ccc5c(ccc6ccc7ccccc7c65)c4)c3)c2)cc1 ; c1ccc(-c2cc(-c3ccc4ccc5ccc6ccccc6c5c4c3)cc(N(c3ccccc3)c3ccc(-c4cccc5ccccc45)cc3)c2)cc1]

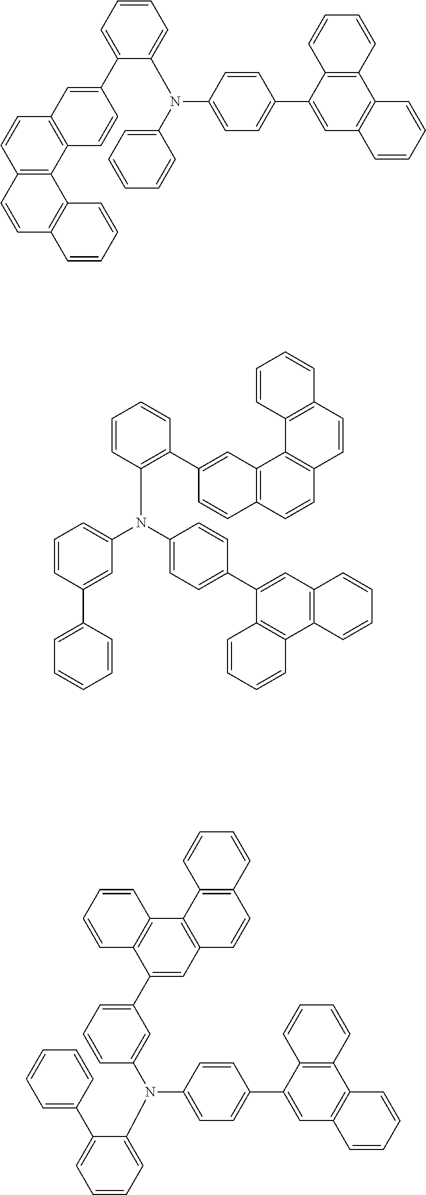 [SMILES: c1ccc(N(c2ccc(-c3cc4ccccc4c4ccccc34)cc2)c2ccccc2-c2ccc3c(ccc4ccc5ccccc5c43)c2)cc1 ; c1ccc(-c2cccc(N(c3ccc(-c4cc5ccccc5c5ccccc45)cc3)c3ccccc3-c3ccc4ccc5ccc6ccccc6c5c4c3)c2)cc1 ; c1ccc(-c2ccccc2N(c2ccc(-c3cc4ccccc4c4ccccc34)cc2)c2cccc(-c3cc4ccc5ccccc5c4c4ccccc34)c2)cc1]

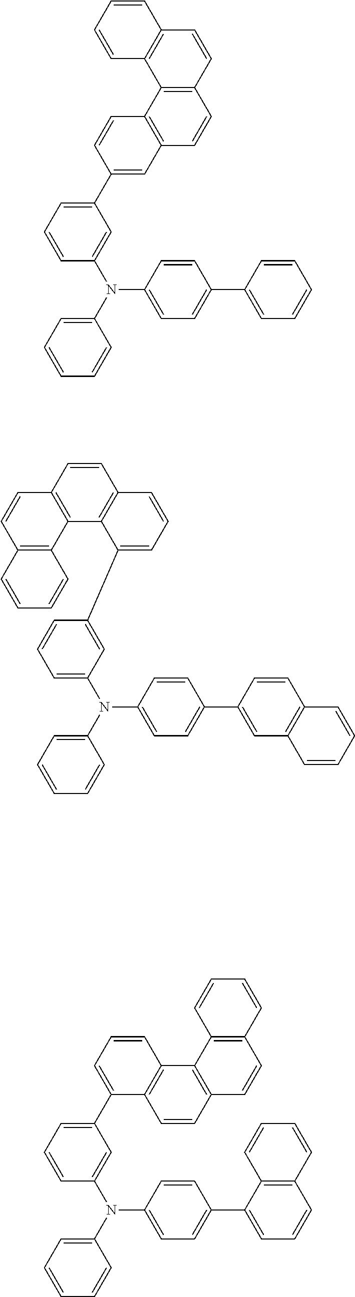 [SMILES: c1ccc(-c2ccc(N(c3ccccc3)c3cccc(-c4ccc5c(ccc6ccc7ccccc7c65)c4)c3)cc2)cc1 ; c1ccc(N(c2ccc(-c3ccc4ccccc4c3)cc2)c2cccc(-c3cccc4ccc5ccc6ccccc6c5c34)c2)cc1 ; c1ccc(N(c2ccc(-c3cccc4ccccc34)cc2)c2cccc(-c3cccc4c3ccc3ccc5ccccc5c34)c2)cc1]

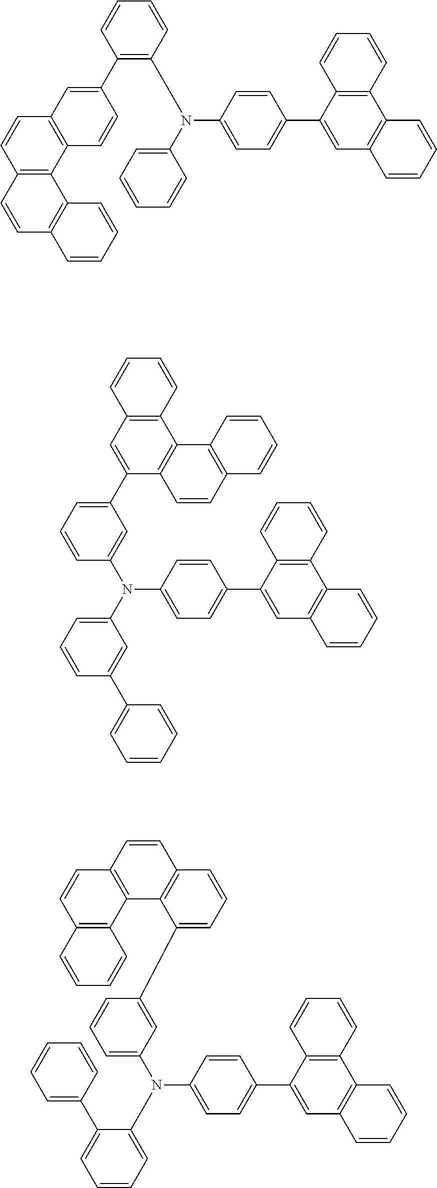 [SMILES: c1ccc(N(c2ccc(-c3cc4ccccc4c4ccccc34)cc2)c2ccccc2-c2ccc3c(ccc4ccc5ccccc5c43)c2)cc1 ; c1ccc(-c2cccc(N(c3ccc(-c4cc5ccccc5c5ccccc45)cc3)c3cccc(-c4cc5ccccc5c5c4ccc4ccccc45)c3)c2)cc1 ; c1ccc(-c2ccccc2N(c2ccc(-c3cc4ccccc4c4ccccc34)cc2)c2cccc(-c3cccc4ccc5ccc6ccccc6c5c34)c2)cc1]

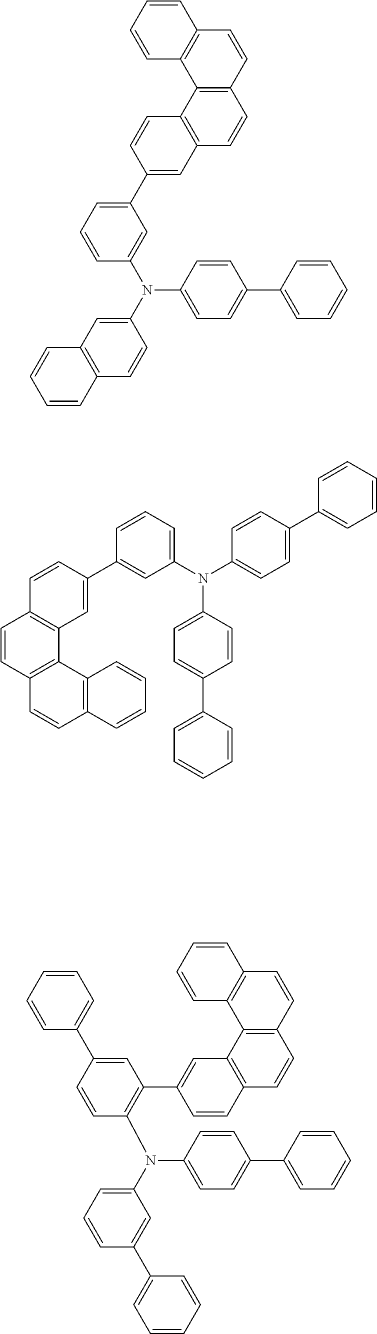 [SMILES: c1ccc(-c2ccc(N(c3cccc(-c4ccc5c(ccc6ccc7ccccc7c65)c4)c3)c3ccc4ccccc4c3)cc2)cc1 ; c1ccc(-c2ccc(N(c3ccc(-c4ccccc4)cc3)c3cccc(-c4ccc5ccc6ccc7ccccc7c6c5c4)c3)cc2)cc1 ; c1ccc(-c2ccc(N(c3cccc(-c4ccccc4)c3)c3ccc(-c4ccccc4)cc3-c3ccc4ccc5ccc6ccccc6c5c4c3)cc2)cc1]

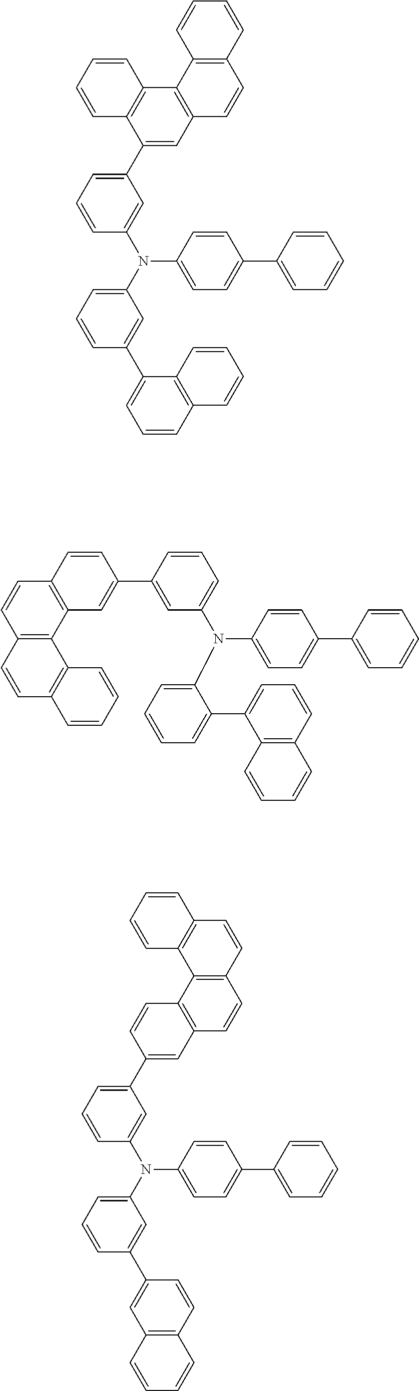 [SMILES: c1ccc(-c2ccc(N(c3cccc(-c4cccc5ccccc45)c3)c3cccc(-c4cc5ccc6ccccc6c5c5ccccc45)c3)cc2)cc1 ; c1ccc(-c2ccc(N(c3cccc(-c4ccc5ccc6ccc7ccccc7c6c5c4)c3)c3ccccc3-c3cccc4ccccc34)cc2)cc1 ; c1ccc(-c2ccc(N(c3cccc(-c4ccc5ccccc5c4)c3)c3cccc(-c4ccc5c(ccc6ccc7ccccc7c65)c4)c3)cc2)cc1]

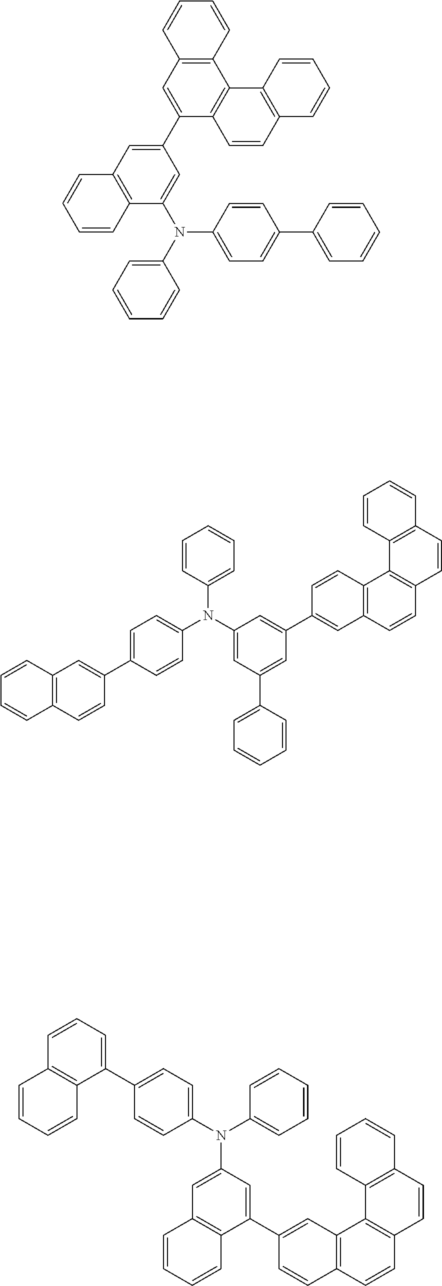 [SMILES: c1ccc(-c2ccc(N(c3ccccc3)c3cc(-c4cc5ccccc5c5c4ccc4ccccc45)cc4ccccc34)cc2)cc1 ; c1ccc(-c2cc(-c3ccc4c(ccc5ccc6ccccc6c54)c3)cc(N(c3ccccc3)c3ccc(-c4ccc5ccccc5c4)cc3)c2)cc1 ; c1ccc(N(c2ccc(-c3cccc4ccccc34)cc2)c2cc(-c3ccc4ccc5ccc6ccccc6c5c4c3)c3ccccc3c2)cc1]

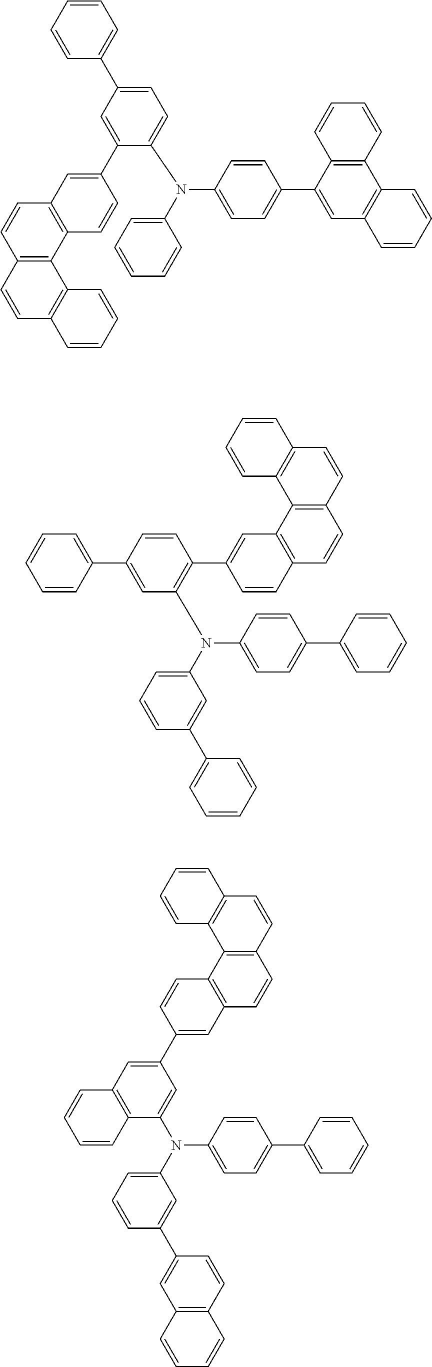 [SMILES: c1ccc(-c2ccc(N(c3ccccc3)c3ccc(-c4cc5ccccc5c5ccccc45)cc3)c(-c3ccc4c(ccc5ccc6ccccc6c54)c3)c2)cc1 ; c1ccc(-c2ccc(N(c3cccc(-c4ccccc4)c3)c3cc(-c4ccccc4)ccc3-c3ccc4ccc5ccc6ccccc6c5c4c3)cc2)cc1 ; c1ccc(-c2ccc(N(c3cccc(-c4ccc5ccccc5c4)c3)c3cc(-c4ccc5c(ccc6ccc7ccccc7c65)c4)cc4ccccc34)cc2)cc1]

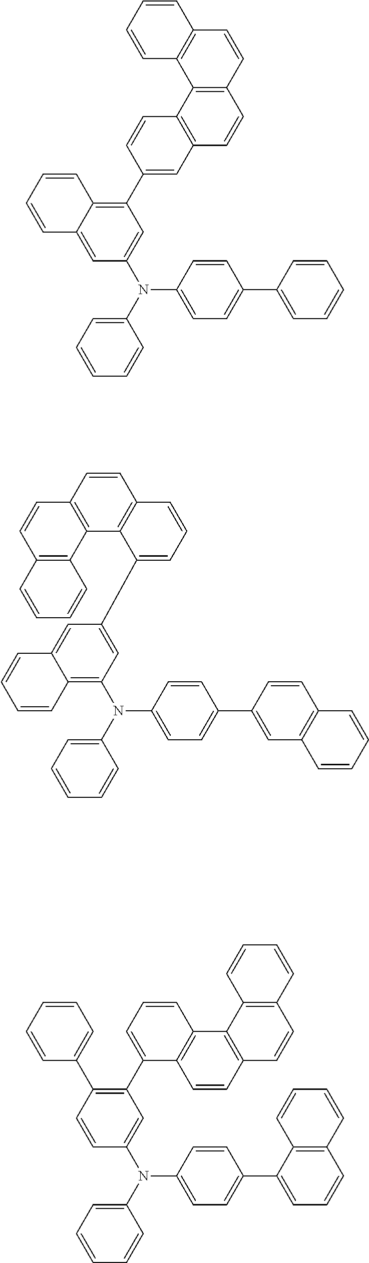 [SMILES: c1ccc(-c2ccc(N(c3ccccc3)c3cc(-c4ccc5c(ccc6ccc7ccccc7c65)c4)c4ccccc4c3)cc2)cc1 ; c1ccc(N(c2ccc(-c3ccc4ccccc4c3)cc2)c2cc(-c3cccc4ccc5ccc6ccccc6c5c34)cc3ccccc23)cc1 ; c1ccc(-c2ccc(N(c3ccccc3)c3ccc(-c4cccc5ccccc45)cc3)cc2-c2cccc3c2ccc2ccc4ccccc4c23)cc1]

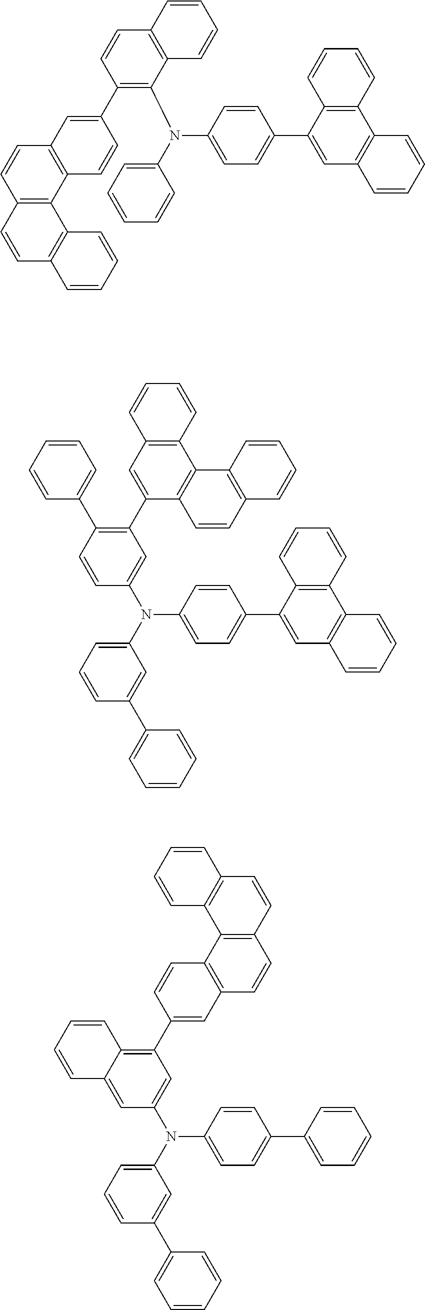 [SMILES: c1ccc(N(c2ccc(-c3cc4ccccc4c4ccccc34)cc2)c2c(-c3ccc4c(ccc5ccc6ccccc6c54)c3)ccc3ccccc23)cc1 ; c1ccc(-c2cccc(N(c3ccc(-c4cc5ccccc5c5ccccc45)cc3)c3ccc(-c4ccccc4)c(-c4cc5ccccc5c5c4ccc4ccccc45)c3)c2)cc1 ; c1ccc(-c2ccc(N(c3cccc(-c4ccccc4)c3)c3cc(-c4ccc5c(ccc6ccc7ccccc7c65)c4)c4ccccc4c3)cc2)cc1]

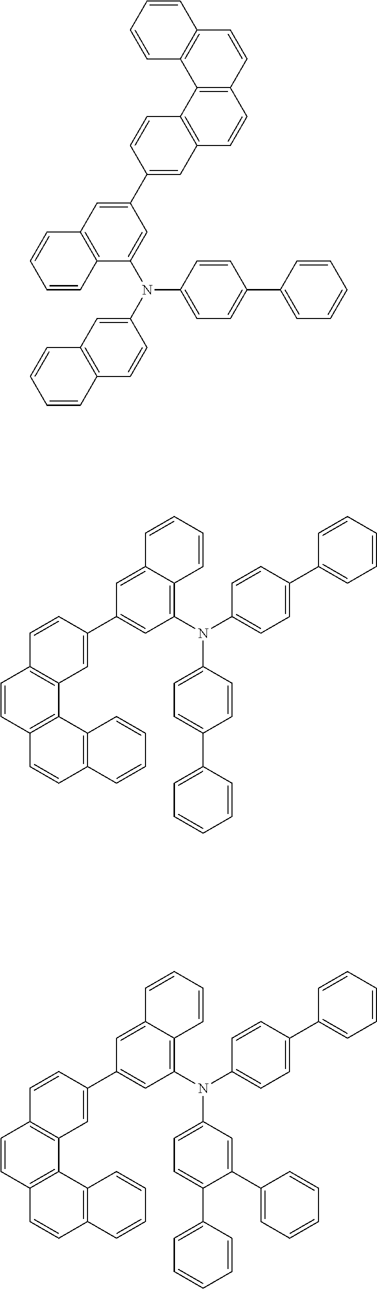 [SMILES: c1ccc(-c2ccc(N(c3ccc4ccccc4c3)c3cc(-c4ccc5c(ccc6ccc7ccccc7c65)c4)cc4ccccc34)cc2)cc1 ; c1ccc(-c2ccc(N(c3ccc(-c4ccccc4)cc3)c3cc(-c4ccc5ccc6ccc7ccccc7c6c5c4)cc4ccccc34)cc2)cc1 ; c1ccc(-c2ccc(N(c3ccc(-c4ccccc4)c(-c4ccccc4)c3)c3cc(-c4ccc5ccc6ccc7ccccc7c6c5c4)cc4ccccc34)cc2)cc1]

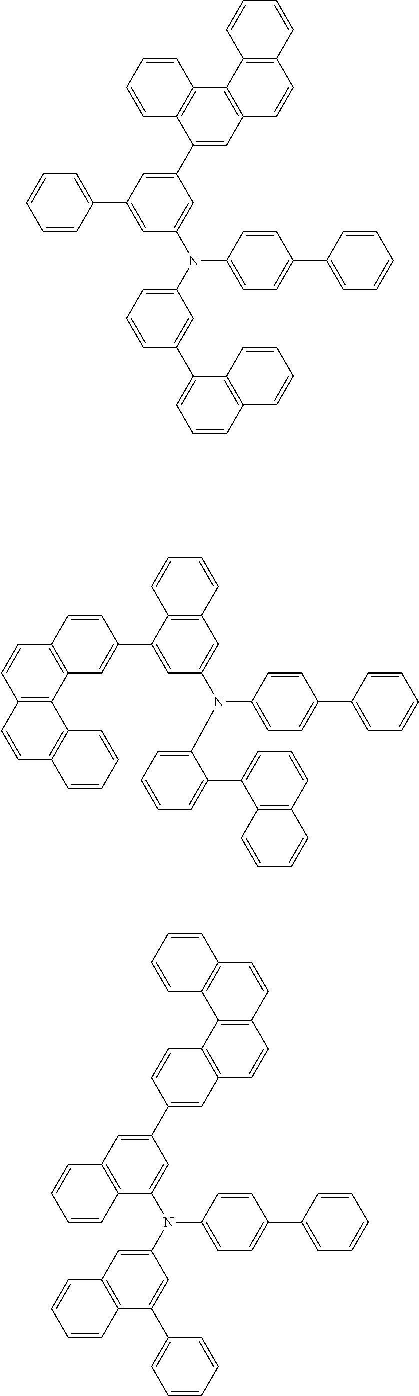 [SMILES: c1ccc(-c2ccc(N(c3cccc(-c4cccc5ccccc45)c3)c3cc(-c4ccccc4)cc(-c4cc5ccc6ccccc6c5c5ccccc45)c3)cc2)cc1 ; c1ccc(-c2ccc(N(c3cc(-c4ccc5ccc6ccc7ccccc7c6c5c4)c4ccccc4c3)c3ccccc3-c3cccc4ccccc34)cc2)cc1 ; c1ccc(-c2ccc(N(c3cc(-c4ccccc4)c4ccccc4c3)c3cc(-c4ccc5c(ccc6ccc7ccccc7c65)c4)cc4ccccc34)cc2)cc1]

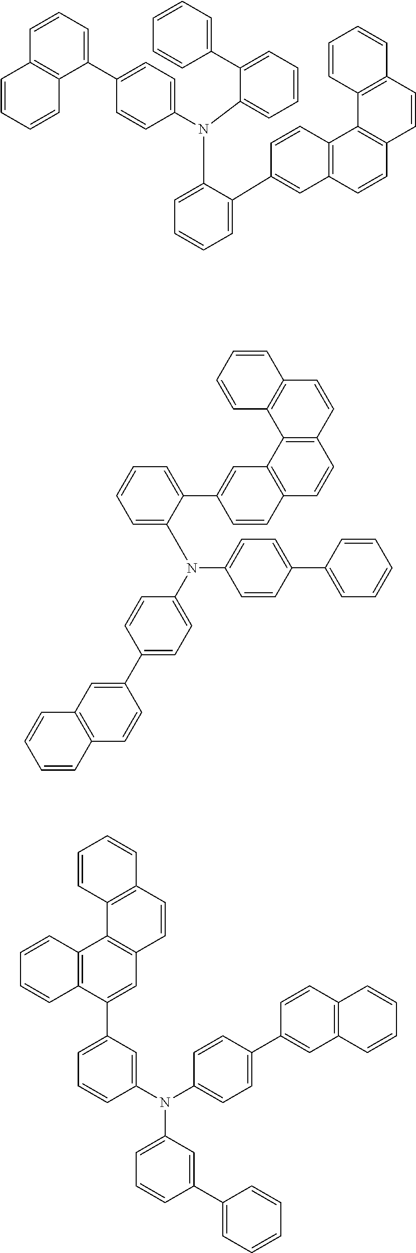 [SMILES: c1ccc(-c2ccccc2N(c2ccc(-c3cccc4ccccc34)cc2)c2ccccc2-c2ccc3c(ccc4ccc5ccccc5c43)c2)cc1 ; c1ccc(-c2ccc(N(c3ccc(-c4ccc5ccccc5c4)cc3)c3ccccc3-c3ccc4ccc5ccc6ccccc6c5c4c3)cc2)cc1 ; c1ccc(-c2cccc(N(c3ccc(-c4ccc5ccccc5c4)cc3)c3cccc(-c4cc5ccc6ccccc6c5c5ccccc45)c3)c2)cc1]

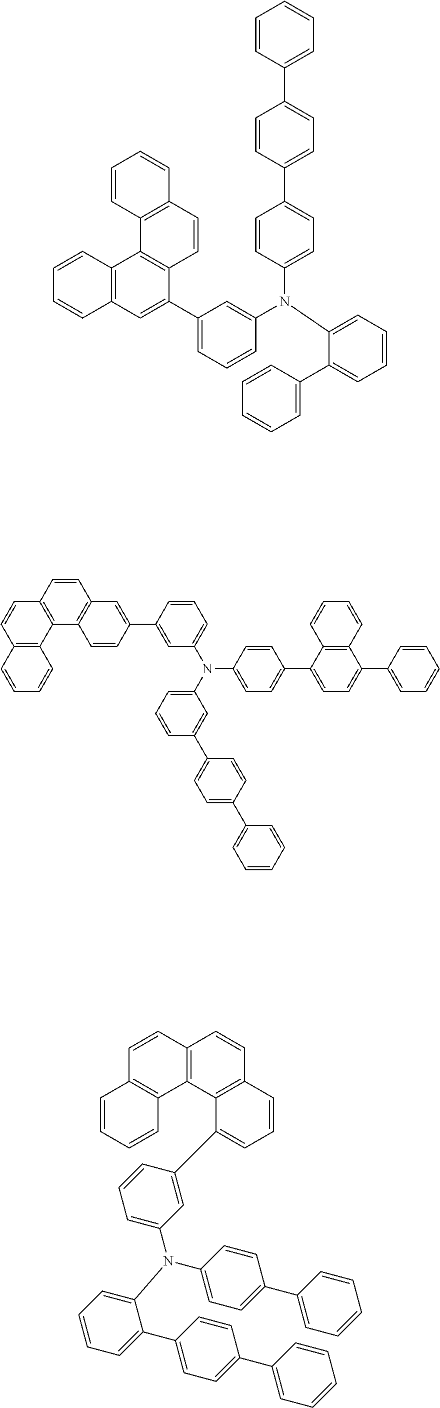 [SMILES: c1ccc(-c2ccc(-c3ccc(N(c4cccc(-c5cc6ccccc6c6c5ccc5ccccc56)c4)c4ccccc4-c4ccccc4)cc3)cc2)cc1 ; c1ccc(-c2ccc(-c3cccc(N(c4ccc(-c5ccc(-c6ccccc6)c6ccccc56)cc4)c4cccc(-c5ccc6c(ccc7ccc8ccccc8c76)c5)c4)c3)cc2)cc1 ; c1ccc(-c2ccc(-c3ccccc3N(c3ccc(-c4ccccc4)cc3)c3cccc(-c4cccc5ccc6ccc7ccccc7c6c45)c3)cc2)cc1]

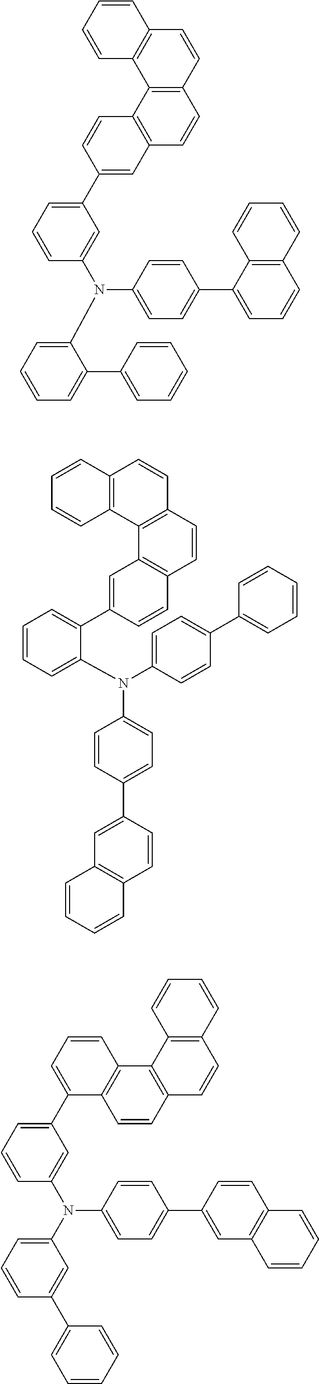 [SMILES: c1ccc(-c2ccccc2N(c2ccc(-c3cccc4ccccc34)cc2)c2cccc(-c3ccc4c(ccc5ccc6ccccc6c54)c3)c2)cc1 ; c1ccc(-c2ccc(N(c3ccc(-c4ccc5ccccc5c4)cc3)c3ccccc3-c3ccc4ccc5ccc6ccccc6c5c4c3)cc2)cc1 ; c1ccc(-c2cccc(N(c3ccc(-c4ccc5ccccc5c4)cc3)c3cccc(-c4cccc5c4ccc4ccc6ccccc6c45)c3)c2)cc1]

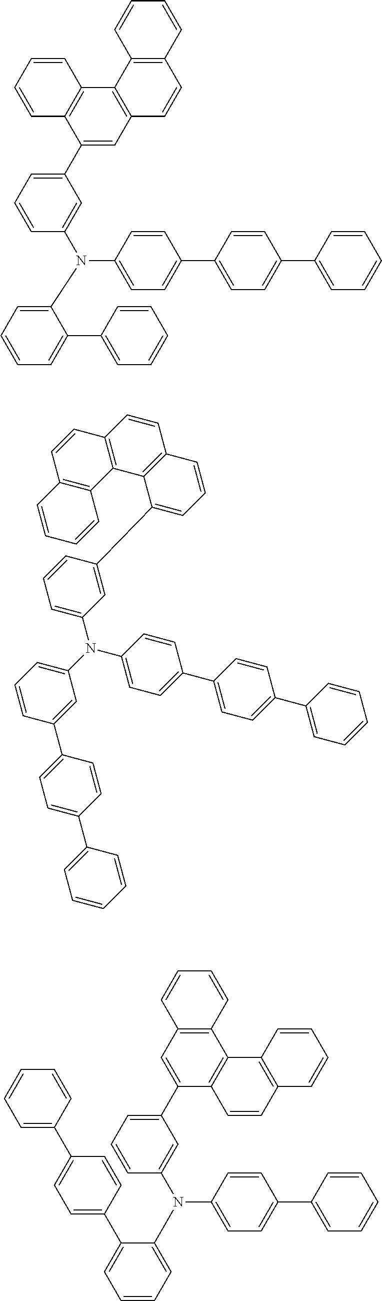 [SMILES: c1ccc(-c2ccc(-c3ccc(N(c4cccc(-c5cc6ccc7ccccc7c6c6ccccc56)c4)c4ccccc4-c4ccccc4)cc3)cc2)cc1 ; c1ccc(-c2ccc(-c3ccc(N(c4cccc(-c5ccc(-c6ccccc6)cc5)c4)c4cccc(-c5cccc6ccc7ccc8ccccc8c7c56)c4)cc3)cc2)cc1 ; c1ccc(-c2ccc(-c3ccccc3N(c3ccc(-c4ccccc4)cc3)c3cccc(-c4cc5ccccc5c5c4ccc4ccccc45)c3)cc2)cc1]

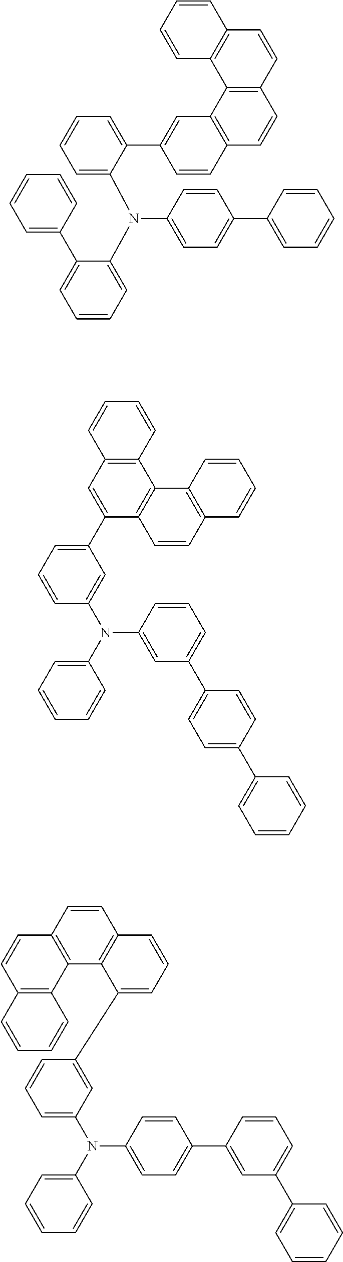 [SMILES: c1ccc(-c2ccc(N(c3ccccc3-c3ccccc3)c3ccccc3-c3ccc4ccc5ccc6ccccc6c5c4c3)cc2)cc1 ; c1ccc(-c2ccc(-c3cccc(N(c4ccccc4)c4cccc(-c5cc6ccccc6c6c5ccc5ccccc56)c4)c3)cc2)cc1 ; c1ccc(-c2cccc(-c3ccc(N(c4ccccc4)c4cccc(-c5cccc6ccc7ccc8ccccc8c7c56)c4)cc3)c2)cc1]

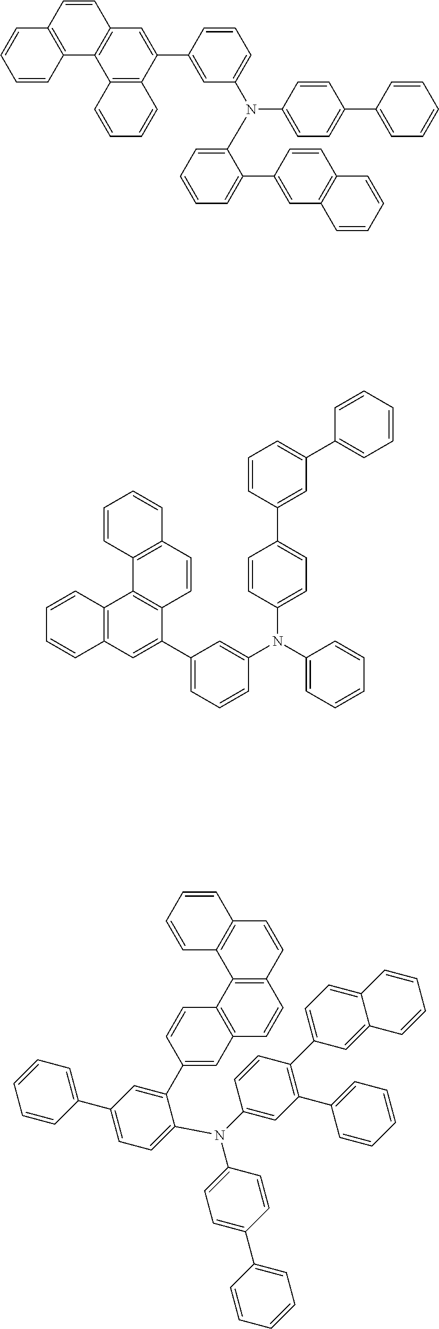 [SMILES: c1ccc(-c2ccc(N(c3cccc(-c4cc5ccc6ccccc6c5c5ccccc45)c3)c3ccccc3-c3ccc4ccccc4c3)cc2)cc1 ; c1ccc(-c2cccc(-c3ccc(N(c4ccccc4)c4cccc(-c5cc6ccccc6c6c5ccc5ccccc56)c4)cc3)c2)cc1 ; c1ccc(-c2ccc(N(c3ccc(-c4ccc5ccccc5c4)c(-c4ccccc4)c3)c3ccc(-c4ccccc4)cc3-c3ccc4c(ccc5ccc6ccccc6c54)c3)cc2)cc1]

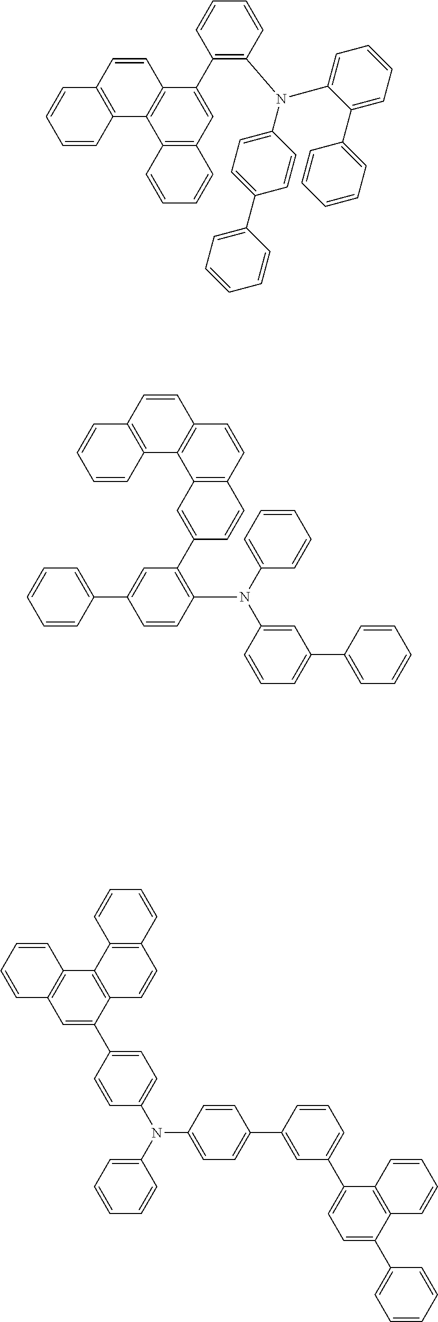 [SMILES: c1ccc(-c2ccc(N(c3ccccc3-c3ccccc3)c3ccccc3-c3cc4ccccc4c4c3ccc3ccccc34)cc2)cc1 ; c1ccc(-c2cccc(N(c3ccccc3)c3ccc(-c4ccccc4)cc3-c3ccc4ccc5ccc6ccccc6c5c4c3)c2)cc1 ; c1ccc(-c2ccc(-c3cccc(-c4ccc(N(c5ccccc5)c5ccc(-c6cc7ccccc7c7c6ccc6ccccc67)cc5)cc4)c3)c3ccccc23)cc1]

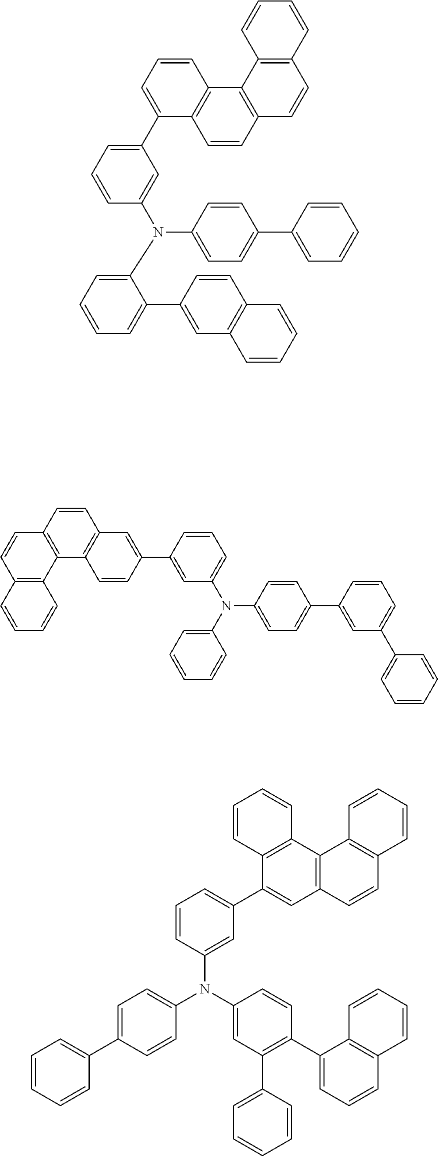 [SMILES: c1ccc(-c2ccc(N(c3cccc(-c4cccc5c4ccc4ccc6ccccc6c45)c3)c3ccccc3-c3ccc4ccccc4c3)cc2)cc1 ; c1ccc(-c2cccc(-c3ccc(N(c4ccccc4)c4cccc(-c5ccc6c(ccc7ccc8ccccc8c76)c5)c4)cc3)c2)cc1 ; c1ccc(-c2ccc(N(c3cccc(-c4cc5ccc6ccccc6c5c5ccccc45)c3)c3ccc(-c4cccc5ccccc45)c(-c4ccccc4)c3)cc2)cc1]

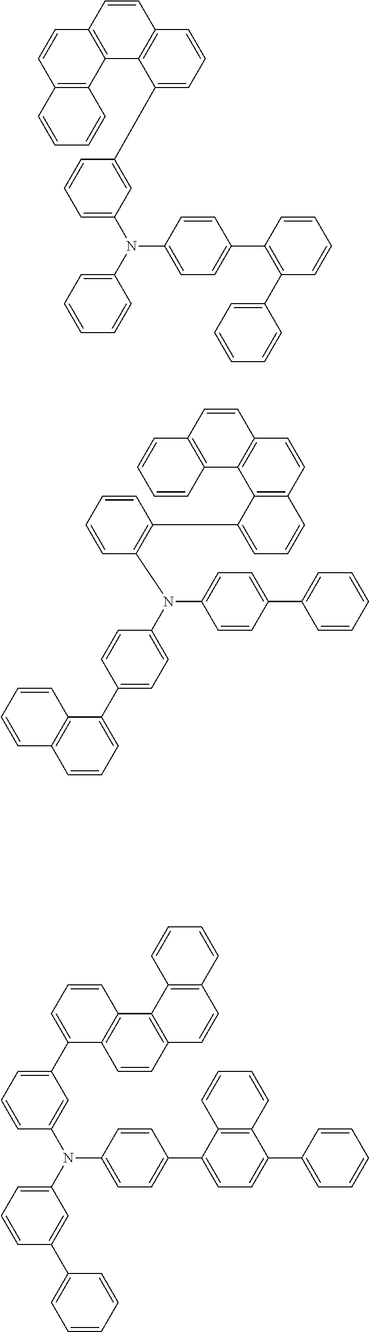 [SMILES: c1ccc(-c2ccccc2-c2ccc(N(c3ccccc3)c3cccc(-c4cccc5ccc6ccc7ccccc7c6c45)c3)cc2)cc1 ; c1ccc(-c2ccc(N(c3ccc(-c4cccc5ccccc45)cc3)c3ccccc3-c3cccc4ccc5ccc6ccccc6c5c34)cc2)cc1 ; c1ccc(-c2cccc(N(c3ccc(-c4ccc(-c5ccccc5)c5ccccc45)cc3)c3cccc(-c4cccc5c4ccc4ccc6ccccc6c45)c3)c2)cc1]

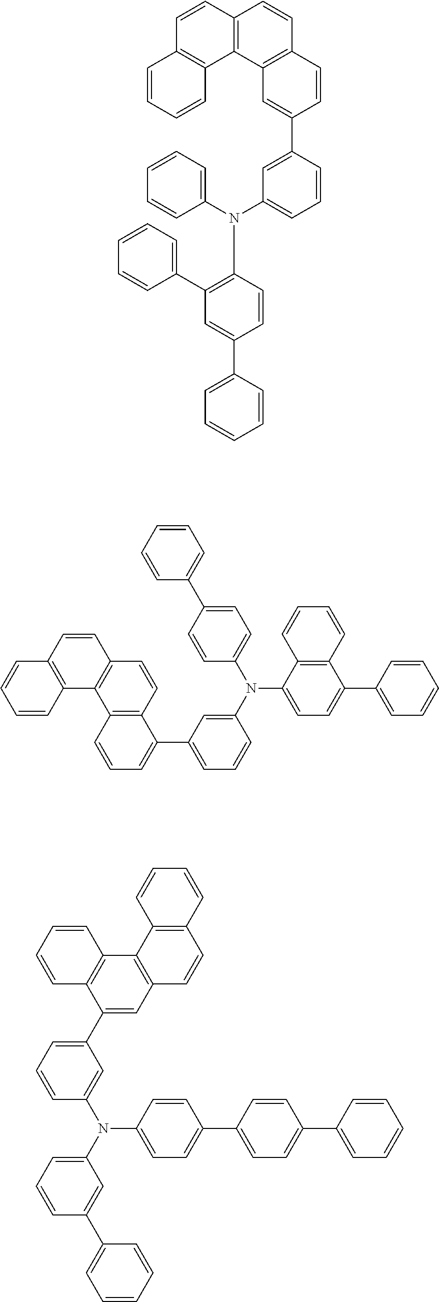 [SMILES: c1ccc(-c2ccc(N(c3ccccc3)c3cccc(-c4ccc5ccc6ccc7ccccc7c6c5c4)c3)c(-c3ccccc3)c2)cc1 ; c1ccc(-c2ccc(N(c3cccc(-c4cccc5c4ccc4ccc6ccccc6c45)c3)c3ccc(-c4ccccc4)c4ccccc34)cc2)cc1 ; c1ccc(-c2ccc(-c3ccc(N(c4cccc(-c5ccccc5)c4)c4cccc(-c5cc6ccc7ccccc7c6c6ccccc56)c4)cc3)cc2)cc1]

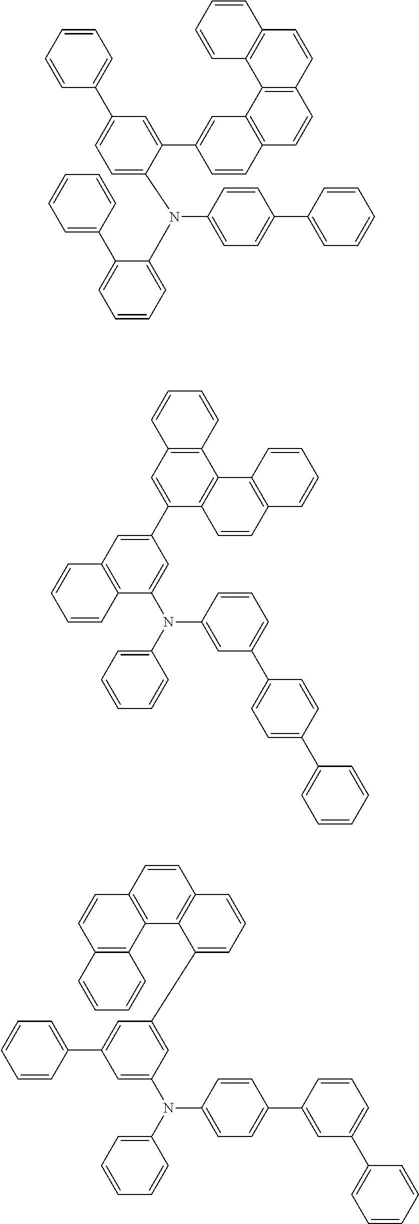 [SMILES: c1ccc(-c2ccc(N(c3ccccc3-c3ccccc3)c3ccc(-c4ccccc4)cc3-c3ccc4ccc5ccc6ccccc6c5c4c3)cc2)cc1 ; c1ccc(-c2ccc(-c3cccc(N(c4ccccc4)c4cc(-c5cc6ccccc6c6c5ccc5ccccc56)cc5ccccc45)c3)cc2)cc1 ; c1ccc(-c2cccc(-c3ccc(N(c4ccccc4)c4cc(-c5ccccc5)cc(-c5cccc6ccc7ccc8ccccc8c7c56)c4)cc3)c2)cc1]

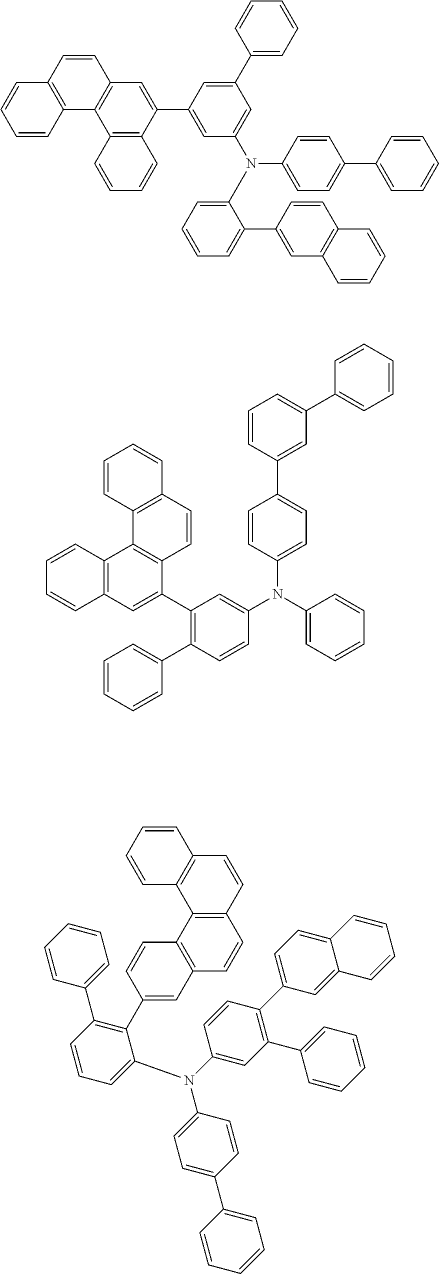 [SMILES: c1ccc(-c2ccc(N(c3cc(-c4ccccc4)cc(-c4cc5ccc6ccccc6c5c5ccccc45)c3)c3ccccc3-c3ccc4ccccc4c3)cc2)cc1 ; c1ccc(-c2cccc(-c3ccc(N(c4ccccc4)c4ccc(-c5ccccc5)c(-c5cc6ccccc6c6c5ccc5ccccc56)c4)cc3)c2)cc1 ; c1ccc(-c2ccc(N(c3ccc(-c4ccc5ccccc5c4)c(-c4ccccc4)c3)c3cccc(-c4ccccc4)c3-c3ccc4c(ccc5ccc6ccccc6c54)c3)cc2)cc1]

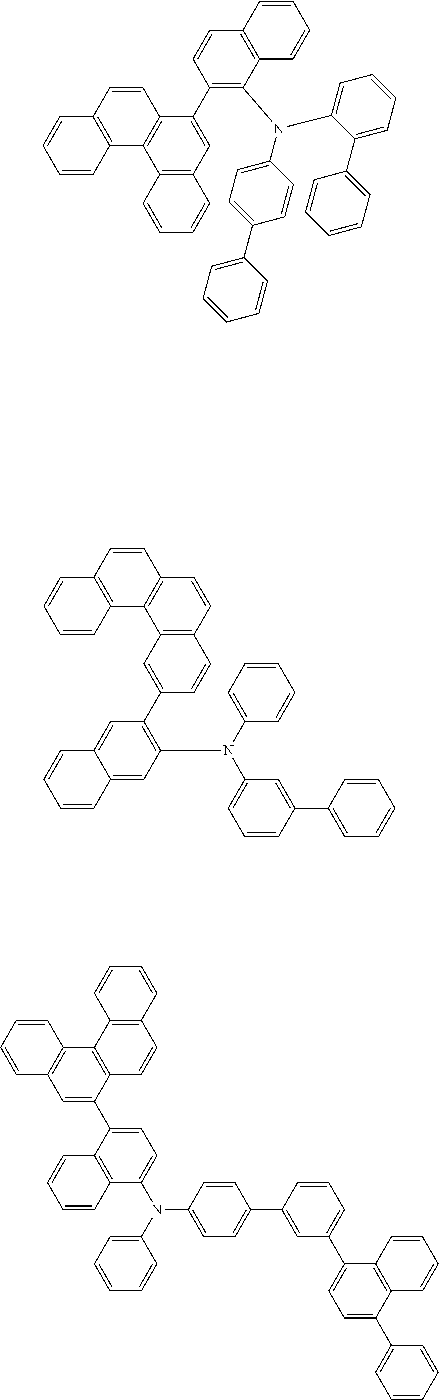 [SMILES: c1ccc(-c2ccc(N(c3ccccc3-c3ccccc3)c3c(-c4cc5ccccc5c5c4ccc4ccccc45)ccc4ccccc34)cc2)cc1 ; c1ccc(-c2cccc(N(c3ccccc3)c3cc4ccccc4cc3-c3ccc4ccc5ccc6ccccc6c5c4c3)c2)cc1 ; c1ccc(-c2ccc(-c3cccc(-c4ccc(N(c5ccccc5)c5ccc(-c6cc7ccccc7c7c6ccc6ccccc67)c6ccccc56)cc4)c3)c3ccccc23)cc1]

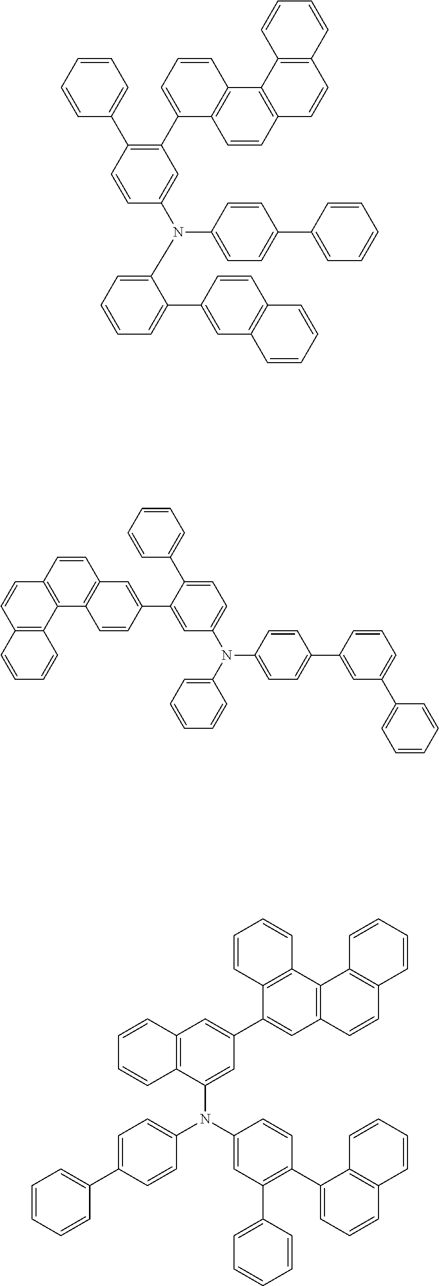 [SMILES: c1ccc(-c2ccc(N(c3ccc(-c4ccccc4)c(-c4cccc5c4ccc4ccc6ccccc6c45)c3)c3ccccc3-c3ccc4ccccc4c3)cc2)cc1 ; c1ccc(-c2cccc(-c3ccc(N(c4ccccc4)c4ccc(-c5ccccc5)c(-c5ccc6c(ccc7ccc8ccccc8c76)c5)c4)cc3)c2)cc1 ; c1ccc(-c2ccc(N(c3ccc(-c4cccc5ccccc45)c(-c4ccccc4)c3)c3cc(-c4cc5ccc6ccccc6c5c5ccccc45)cc4ccccc34)cc2)cc1]

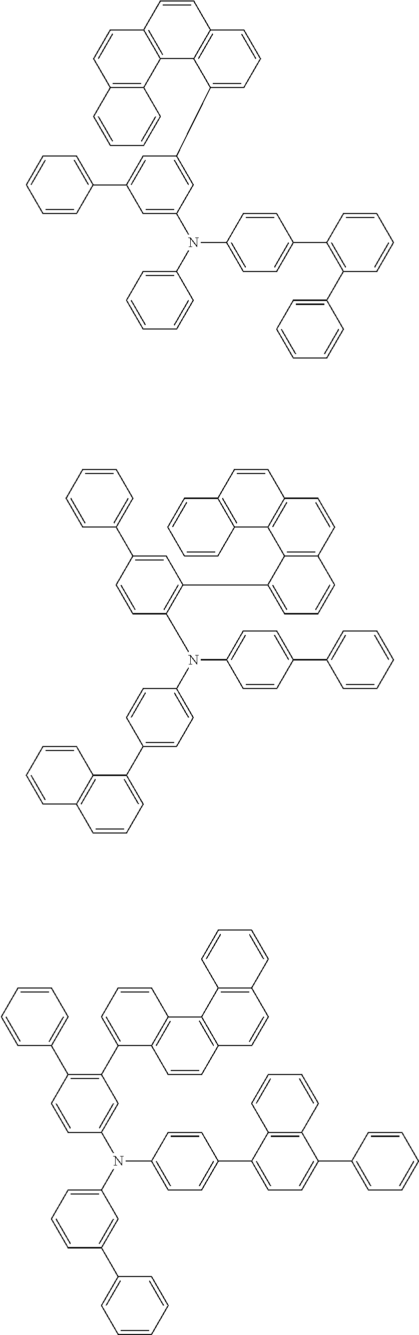 [SMILES: c1ccc(-c2cc(-c3cccc4ccc5ccc6ccccc6c5c34)cc(N(c3ccccc3)c3ccc(-c4ccccc4-c4ccccc4)cc3)c2)cc1 ; c1ccc(-c2ccc(N(c3ccc(-c4cccc5ccccc45)cc3)c3ccc(-c4ccccc4)cc3-c3cccc4ccc5ccc6ccccc6c5c34)cc2)cc1 ; c1ccc(-c2cccc(N(c3ccc(-c4ccc(-c5ccccc5)c5ccccc45)cc3)c3ccc(-c4ccccc4)c(-c4cccc5c4ccc4ccc6ccccc6c45)c3)c2)cc1]

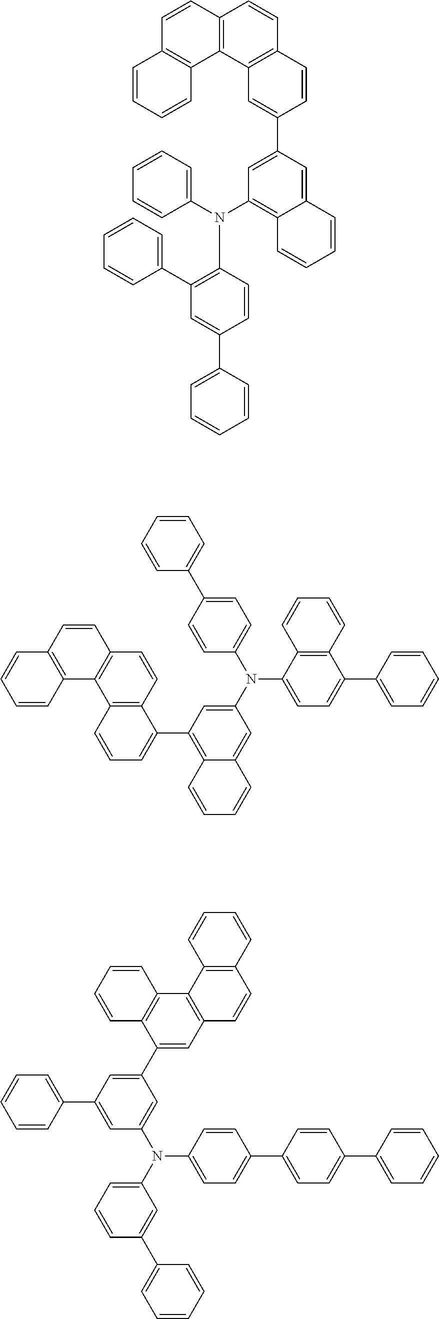 [SMILES: c1ccc(-c2ccc(N(c3ccccc3)c3cc(-c4ccc5ccc6ccc7ccccc7c6c5c4)cc4ccccc34)c(-c3ccccc3)c2)cc1 ; c1ccc(-c2ccc(N(c3cc(-c4cccc5c4ccc4ccc6ccccc6c45)c4ccccc4c3)c3ccc(-c4ccccc4)c4ccccc34)cc2)cc1 ; c1ccc(-c2ccc(-c3ccc(N(c4cccc(-c5ccccc5)c4)c4cc(-c5ccccc5)cc(-c5cc6ccc7ccccc7c6c6ccccc56)c4)cc3)cc2)cc1]

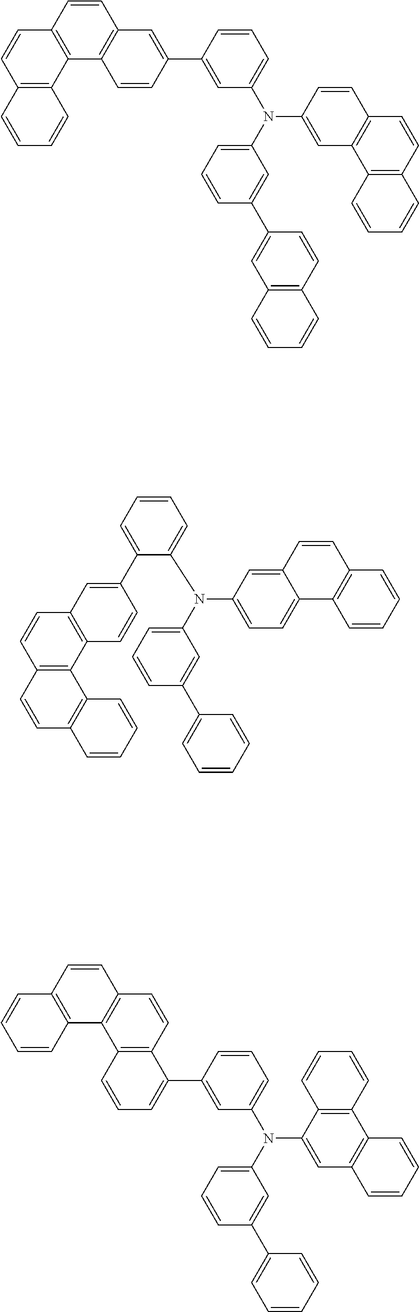 [SMILES: c1cc(-c2ccc3ccccc3c2)cc(N(c2cccc(-c3ccc4c(ccc5ccc6ccccc6c54)c3)c2)c2ccc3ccc4ccccc4c3c2)c1 ; c1ccc(-c2cccc(N(c3ccc4c(ccc5ccccc54)c3)c3ccccc3-c3ccc4c(ccc5ccc6ccccc6c54)c3)c2)cc1 ; c1ccc(-c2cccc(N(c3cccc(-c4cccc5c4ccc4ccc6ccccc6c45)c3)c3cc4ccccc4c4ccccc34)c2)cc1]

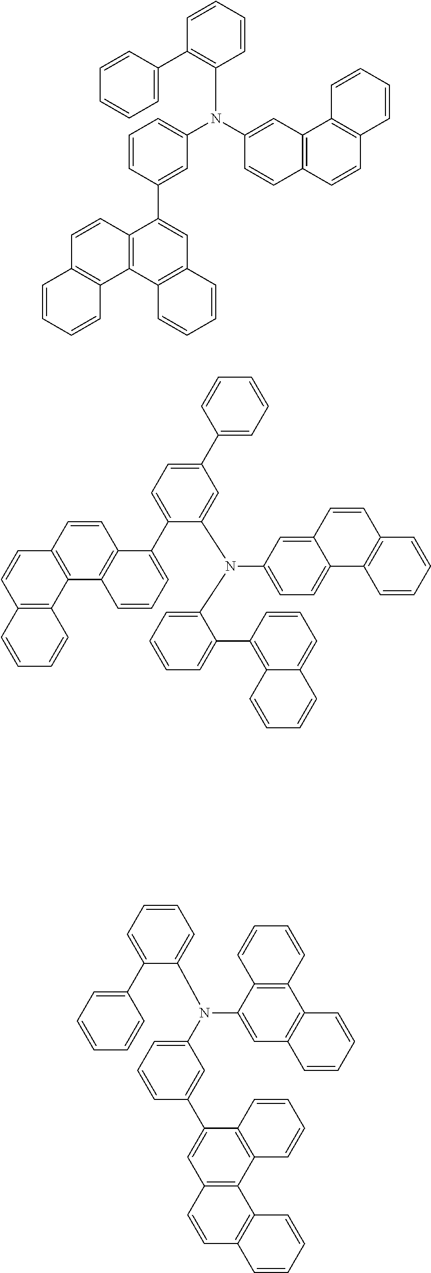 [SMILES: c1ccc(-c2ccccc2N(c2cccc(-c3cc4ccccc4c4c3ccc3ccccc34)c2)c2ccc3ccc4ccccc4c3c2)cc1 ; c1ccc(-c2ccc(-c3cccc4c3ccc3ccc5ccccc5c34)c(N(c3ccc4c(ccc5ccccc54)c3)c3ccccc3-c3cccc4ccccc34)c2)cc1 ; c1ccc(-c2ccccc2N(c2cccc(-c3cc4ccc5ccccc5c4c4ccccc34)c2)c2cc3ccccc3c3ccccc23)cc1]

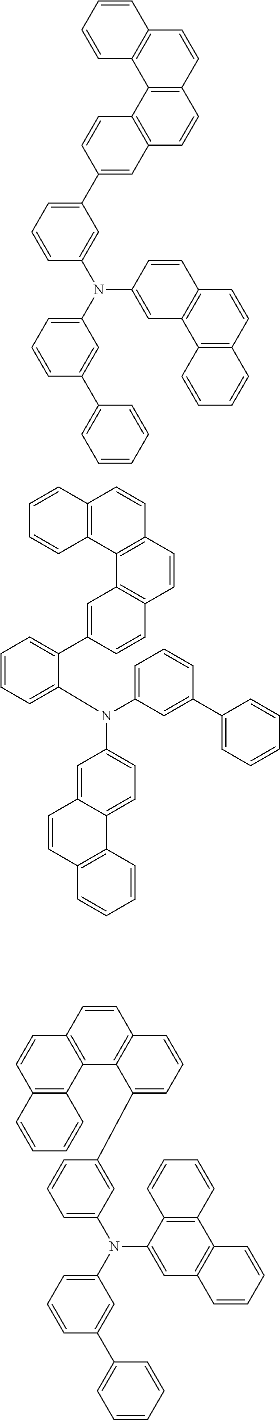

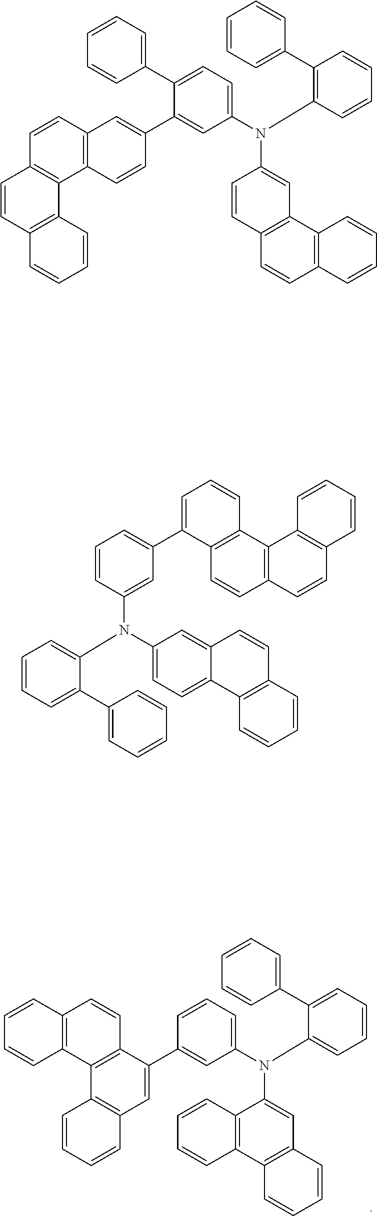 [SMILES: c1ccc(-c2ccc(N(c3ccc4ccc5ccccc5c4c3)c3ccccc3-c3ccccc3)cc2-c2ccc3c(ccc4ccc5ccccc5c43)c2)cc1 ; c1ccc(-c2ccccc2N(c2cccc(-c3cccc4c3ccc3ccc5ccccc5c34)c2)c2ccc3c(ccc4ccccc43)c2)cc1 ; c1ccc(-c2ccccc2N(c2cccc(-c3cc4ccccc4c4c3ccc3ccccc34)c2)c2cc3ccccc3c3ccccc23)cc1]

The compounds of chemical Formula 1, wherein any one of R₁ to R₂ is a substituent of the following Chemical Formula 2 and the rest is hydrogen, can be prepared by a preparation methods as shown in the following Reaction Scheme 1 as an example, an other remaining compounds can be prepared in a similar manner.

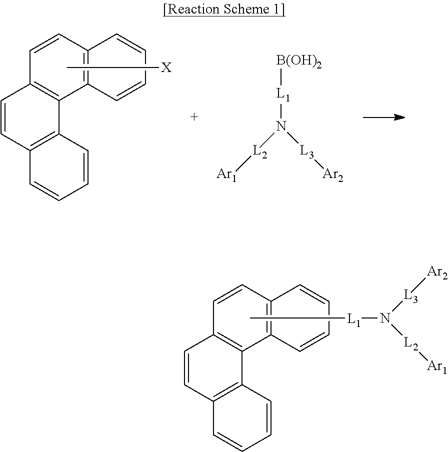

In Reaction Scheme 1, L₁ to L₃, Ar₁ and Ar₂ are as defined in Chemical Formula 1, X is halogen, and preferably X is chloro or bromo.

Reaction Scheme 1 is a Suzuki coupling reaction, which is preferably carried out in the presence of a palladium catalyst and a base, and a reactive group for the Suzuki coupling reaction can be modified as known in the art. The above preparation method can be further embodied in Preparation Examples described hereinafter.

In another embodiment of the present disclosure, there is provided an organic light emitting device including the compound of Chemical Formula 1. In one example, the present disclosure provides an organic light emitting device comprising: a first electrode; a second electrode that is provided opposite to the first electrode; and one or more organic material layers that are provided between the first electrode and the second electrode, wherein one or more layers of the organic material layers include the compound of Chemical Formula 1.

The organic material layer of the organic light emitting device of the present disclosure can have a single-layer structure, or it can have a multilayered structure in which two or more organic material layers are stacked. For example, the organic light emitting device of the present disclosure can have a structure comprising a hole injection layer, a hole transport layer, a light emitting layer, an electron transport layer, an electron injection layer and the like as the organic material layer. However, the structure of the organic light emitting device is not limited thereto, and it can include a smaller number of organic material layers.

Further, the organic material layer can include a light emitting layer, wherein the light emitting layer can include the compound of Chemical Formula 1.

Further, the organic material layer can include a hole transport layer, a hole injection layer, or a layer for simultaneously performing hole transport and injection, wherein the hole transport layer, the hole injection layer, or the layer for simultaneously performing hole transport and injection can include the compound of Chemical Formula 1.

Further, the organic material layer can include an electron injection layer, an electron transport layer, or an electron injection and transport layer, wherein the electron injection layer, the electron transport layer, or the electron injection and transport layer can include the compound of Chemical Formula 1.

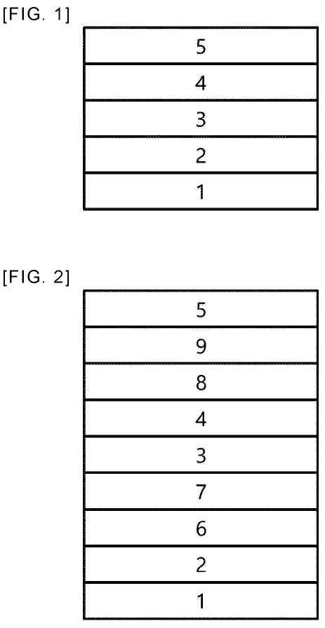

Further, the organic light emitting device according to the present disclosure can be a normal type organic light emitting device in which an anode, one or more organic material layers and a cathode are sequentially stacked on a substrate. Further, the organic light emitting device according to the present disclosure can be an inverted type organic light emitting device in which a cathode, one or more organic material layers and an anode are sequentially stacked on a substrate. For example, the structure of an organic light emitting device according to an embodiment of the present disclosure is illustrated in FIGS. 1 and 2.

FIG. 1 shows an example of an organic light emitting device comprising a substrate 1, an anode 2, an electron blocking layer 3, a light emitting layer 4 and a cathode 5. FIG. 2 shows an example of an organic light emitting device comprising a substrate 1, an anode 2, a hole injection layer 6, a hole transport layer 7, an electron blocking layer 3, a light emitting layer 4, a hole blocking layer 8, an electron transport and injection layer 9 and a cathode 5. In such a structure, the compound of Chemical Formula 1 can be included in the hole transport layer, the electron blocking layer or the light emitting layer.

The organic light emitting device according to the present disclosure can be manufactured by materials and methods known in the art, except that at least one of the organic material layers includes the compound of Chemical Formula 1. Further, when the organic light emitting device includes a plurality of organic material layers, the organic material layers can be formed of the same material or different materials.

For example, the organic light emitting device according to the present disclosure can be manufactured by sequentially stacking a first electrode, an organic material layer and a second electrode on a substrate. In this case, the organic light emitting device can be manufactured by depositing a metal, metal oxides having conductivity, or an alloy thereof on the substrate using a PVD (physical vapor deposition) method such as a sputtering method or an e-beam evaporation method to form an anode, forming organic material layers including the hole injection layer, the hole transport layer, the light emitting layer and the electron transport layer thereon, and then depositing a material that can be used as the cathode thereon. In addition to such a method, the organic light emitting device can be manufactured by sequentially depositing a cathode material, an organic material layer and an anode material on a substrate.

Further, the compound of Chemical Formula 1 can be formed into an organic layer by a solution coating method as well as a vacuum deposition method at the time of manufacturing an organic light emitting device. Herein, the solution coating method means a spin coating, a dip coating, a doctor blading, an inkjet printing, a screen printing, a spray method, a roll coating, or the like, but is not limited thereto.

In addition to such a method, the organic light emitting device can be manufactured by sequentially depositing a cathode material, an organic material layer and an anode material on a substrate (International Publication WO2003/012890). However, the manufacturing method is not limited thereto.

As an example, the first electrode is an anode, and the second electrode is a cathode, or alternatively, the first electrode is a cathode and the second electrode is an anode.

As the anode material, generally, a material having a large work function is preferably used so that holes can be smoothly injected into the organic material layer. Specific examples of the anode material include metals such as vanadium, chrome, copper, zinc, and gold, or an alloy thereof; metal oxides such as zinc oxides, indium oxides, indium tin oxides (ITO), and indium zinc oxides (IZO); a combination of metals and oxides, such as ZnO:Al or SnO₂:Sb; conductive compounds such as poly(3-methylthiophene), poly[3,4-(ethylene-1,2-dioxy)thiophene](PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/Al or LiO₂/Al, and the like, but are not limited thereto.

The hole injection layer is a layer for injecting holes from the electrode, and the hole injection material is preferably a compound which has a capability of transporting the holes, thus has a hole injecting effect in the anode and an excellent hole injecting effect to the light emitting layer or the light emitting material, prevents excitons produced in the light emitting layer from moving to an electron injection layer or the electron injection material, and further is excellent in the ability to form a thin film. It is preferable that a HOMO (highest occupied molecular orbital) of the hole injection material is between the work function of the anode material and a HOMO of a peripheral organic material layer. Specific examples of the hole injection material include metal porphyrine, oligothiophene, an arylamine-based organic material, a hexanitrilehexaazatriphenylene-based organic material, a quinacridone-based organic material, a perylene-based organic material, anthraquinone, polyaniline and polythiophene-based conductive polymer, and the like, but are not limited thereto.

The hole transport layer is a layer that receives holes from a hole injection layer and transports the holes to the light emitting layer. The hole transport layer is suitably a material having large mobility to the holes, which can receive holes from the anode or the hole injection layer and transfer the holes to the light emitting layer. Specific examples thereof include an arylamine-based organic material, a conductive polymer, a block copolymer in which a conjugate portion and a non-conjugate portion are present together, and the like, but are not limited thereto. Preferably, the compound of Chemical Formula 1 can be used as a material for the hole transport layer.

The electron blocking layer is a layer provided between the hole transport layer and the light emitting layer in order to prevent the holes injected in the cathode from being transferred to the hole transport layer without being recombined in the light emitting layer, which can also be referred to as an electron inhibition layer. The electron blocking layer is preferably a material having the smaller electron affinity than the electron transport layer. Preferably, the compound of Chemical Formula 1 can be used as a material for the electron blocking layer.

The light emitting material is preferably a material which can receive holes and electrons transported from a hole transport layer and an electron transport layer, respectively, and combine the holes and the electrons to emit light in a visible ray region, and has good quantum efficiency to fluorescence or phosphorescence. Specific examples of the light emitting material include an 8-hydroxy-quinoline aluminum complex (Alq₃); a carbazole-based compound; a dimerized styryl compound; BAlq; a 10-hydroxybenzoquinoline-metal compound; a benzoxazole, benzothiazole and benzimidazole-based compound; a poly(p-phenylenevinylene)(PPV)-based polymer; a spiro compound; polyfluorene, rubrene, and the like, but are not limited thereto.

The light emitting layer can include a host material and a dopant material. The host material can be a fused aromatic ring derivative, a heterocycle-containing compound or the like. Specific examples of the fused aromatic ring derivatives include anthracene derivatives, pyrene derivatives, naphthalene derivatives, pentacene derivatives, phenanthrene compounds, fluoranthene compounds, and the like. Examples of the heterocyclic-containing compounds include carbazole derivatives, dibenzofuran derivatives, ladder-type furan compounds, pyrimidine derivatives, and the like, but are not limited thereto. Preferably, the compound of Chemical Formula 1 can be included as a host material.

Examples of the dopant material include an aromatic amine derivative, a styrylamine compound, a boron complex, a fluoranthene compound, a metal complex, and the like. Specifically, the aromatic amine derivative is a substituted or unsubstituted fused aromatic ring derivative having an arylamino group, and examples thereof include pyrene, anthracene, chrysene, periflanthene and the like, which have an arylamino group. The styrylamine compound is a compound where at least one arylvinyl group is substituted in substituted or unsubstituted arylamine, in which one or two or more substituent groups selected from the group consisting of an aryl group, a silyl group, an alkyl group, a cycloalkyl group, and an arylamino group are substituted or unsubstituted. Specific examples thereof include styrylamine, styryldiamine, styryltriamine, styryltetramine, and the like, but are not limited thereto. Further, the metal complex includes an iridium complex, a platinum complex, and the like, but is not limited thereto.

The hole blocking layer is a layer provided between the electron transport layer and the light emitting layer in order to prevent the holes injected in the anode from being transferred to the electron transport layer without being recombined in the light emitting layer, which can also be referred to as a hole inhibition layer. The hole blocking layer is preferably a material having the large ionization energy.

The electron transport layer is a layer which receives electrons from an electron injection layer and transports the electrons to a light emitting layer, and an electron transport material is suitably a material which can receive electrons well from a cathode and transfer the electrons to a light emitting layer, and has a large mobility for electrons. Specific examples of the electron transport material include: an Al complex of 8-hydroxyquinoline, a complex including Alq₃, an organic radical compound, a hydroxyflavone-metal complex, and the like, but are not limited thereto. The electron transport layer can be used with any desired cathode material, as used according to the related art. In particular, appropriate examples of the cathode material are a typical material which has a low work function, followed by an aluminum layer or a silver layer. Specific examples thereof include cesium, barium, calcium, ytterbium, and samarium, in each case followed by an aluminum layer or a silver layer.

The electron injection layer is a layer which injects electrons from an electrode, and is preferably a compound which has a capability of transporting electrons, has an effect of injecting electrons from a cathode and an excellent effect of injecting electrons into a light emitting layer or a light emitting material, prevents excitons produced from the light emitting layer from moving to a hole injection layer, and is also excellent in the ability to form a thin film. Specific examples of the electron injection layer include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

Examples of the metal complex compound include 8-hydroxyquinolinato lithium, bis(8-hydroxyquinolinato)zinc, bis(8-hydroxyquinolinato)copper, bis(8-hydroxyquinolinato)manganese, tris(8-hydroxyquinolinato)aluminum, tris(2-methyl-8-hydroxy-quinolinato)aluminum, tris(8-hydroxyquinolinato)gallium, bis(10-hydroxybenzo[h]quinolinato)beryllium, bis(10-hydroxybenzo[h]-quinolinato)zinc, bis(2-methyl-8-quinolinato)chlorogallium, bis(2-methyl-8-quinolinato)(o-cresolato)gallium, bis(2-methyl-8-quinolinato)(1-naphtholato)aluminum, bis(2-methyl-8-quinolinato)(2-naphtholato)gallium, and the like, but are not limited thereto.

On the other hand, in the present disclosure, the “electron injection and transport layer” or the “electron transport and injection layer” is a layer that performs both the roles of the electron injection layer and the electron transport layer, and the materials that perform the roles of each layer can be used alone or in combination, without being limited thereto.

The organic light emitting device according to the present disclosure can be a frontside emission type, a backside emission type, or a double-sided emission type based on the used material.

Meanwhile, the organic light emitting device according to the present disclosure can be a bottom emission device, a top emission device, or a double-sided light emitting device, and particularly, can be a bottom emission device that requires relatively high luminous efficiency.

Hereinafter, preferred examples are presented to assist in the understanding of the present disclosure. However, the following examples are only provided for a better understanding of the present disclosure, and is not intended to limit the content of the present disclosure.

### PREPARATION EXAMPLE

### Preparation Example 1

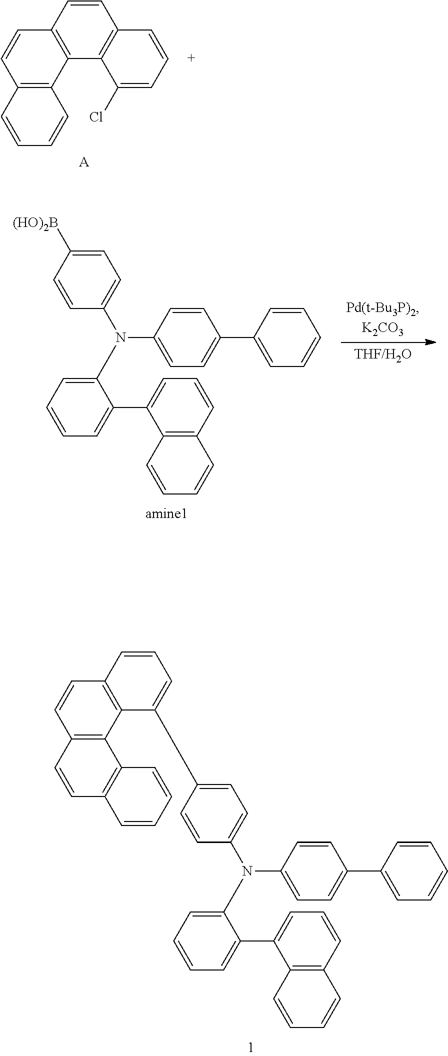

Compound A (15 g, 57.1 mmol) and Compound amine1 (29.5 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 11 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 11.9 g of Compound 1. (Yield: 31%, MS: [M+H]⁺=674)

### Preparation Example 2

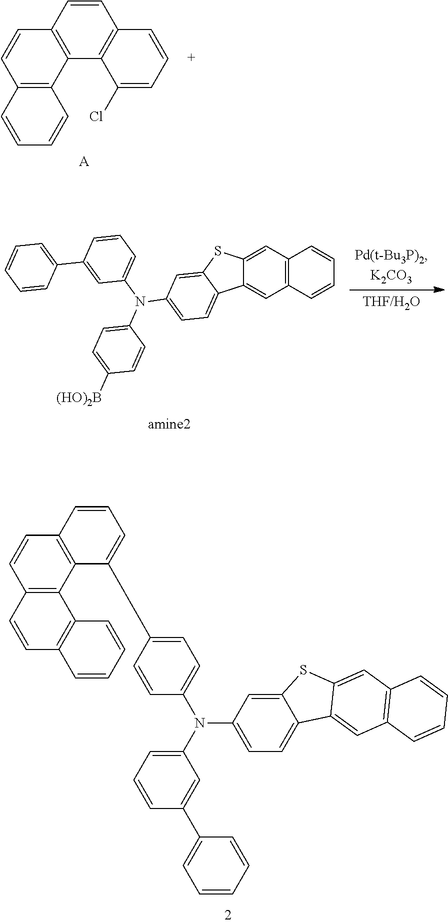

Compound A (15 g, 57.1 mmol) and Compound amine2 (31.3 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 8 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 15.3 g of Compound 2. (Yield: 38%, MS: [M+H]⁺=704)

### Preparation Example 3

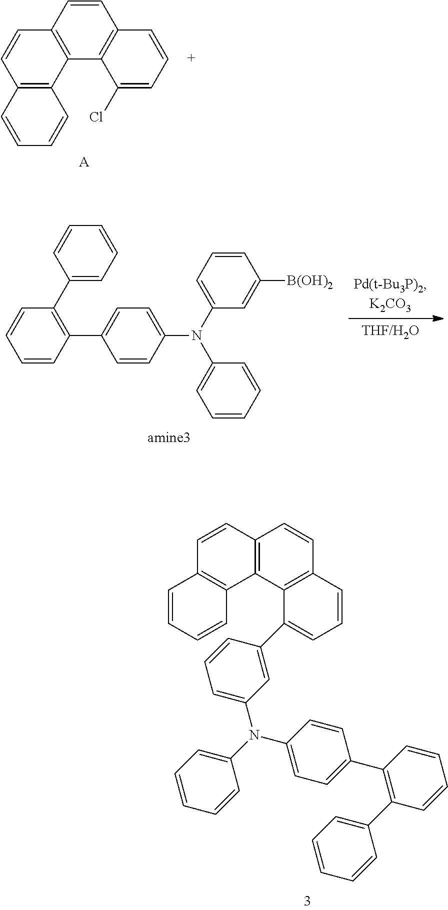

Compound A (15 g, 57.1 mmol) and Compound amine3 (26.5 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 9 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 11.4 g of Compound 3. (Yield: 32%, MS: [M+H]⁺=624)

### Preparation Example 4

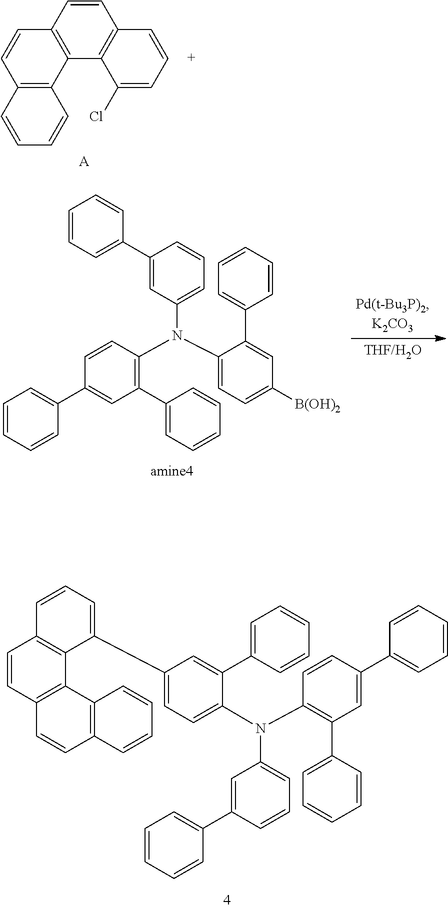

Compound A (15 g, 57.1 mmol) and Compound amine4 (35.6 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 12 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 14.6 g of Compound 4. (Yield: 33%, MS: [M+H]⁺=776)

### Preparation Example 5

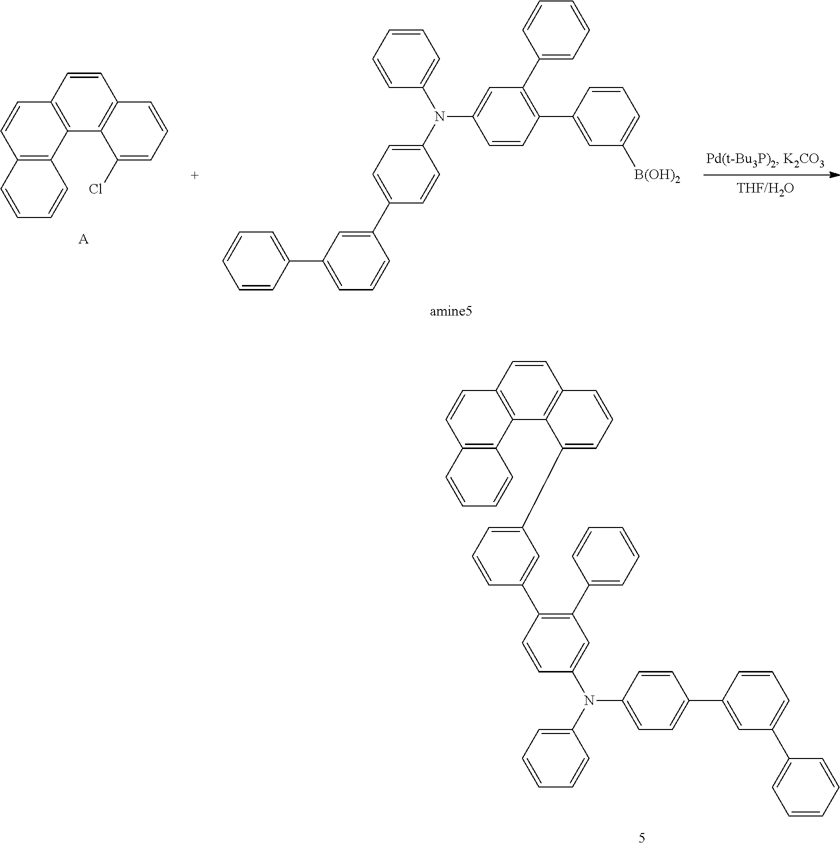

Compound A (15 g, 57.1 mmol) and Compound amine5 (35.6 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 12 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 15 g of Compound 5. (Yield: 34%, MS: [M+H]⁺=776)

### Preparation Example 6

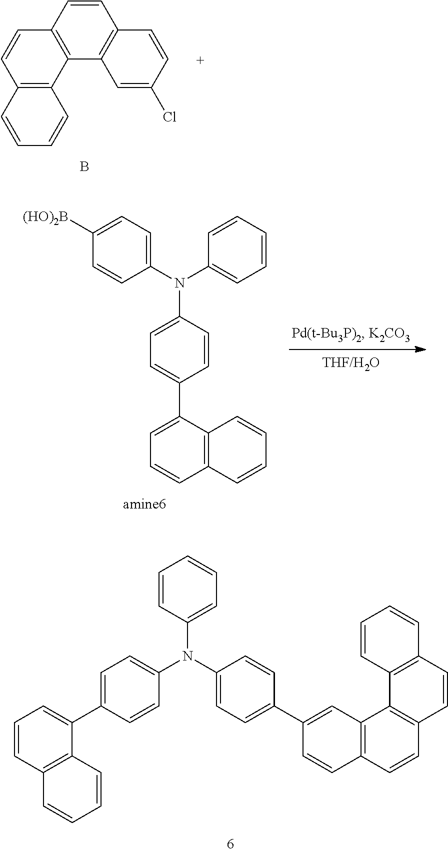

Compound B (15 g, 57.1 mmol) and Compound amine6 (24.9 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 12 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 24.6 g of Compound 6. (Yield: 72%, MS: [M+H]⁺=598)

### Preparation Example 7

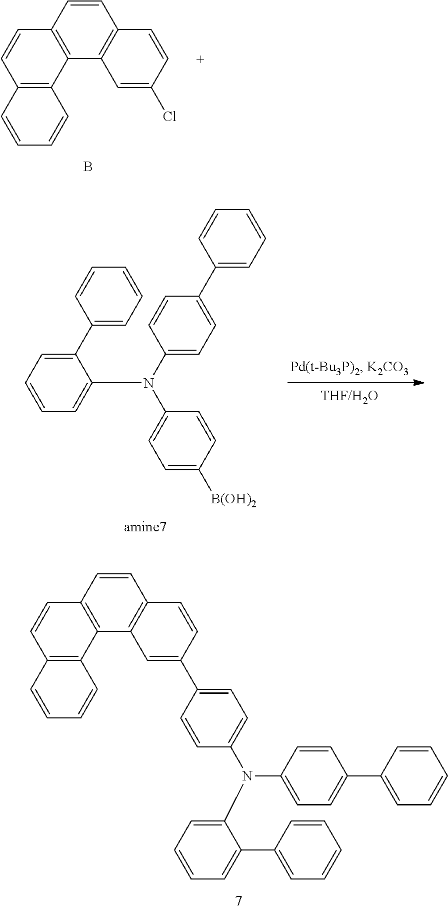

Compound B (15 g, 57.1 mmol) and Compound amine7 (26.5 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 11 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 26.7 g of Compound 7. (Yield: 75%, MS: [M+H]⁺=624)

### Preparation Example 8

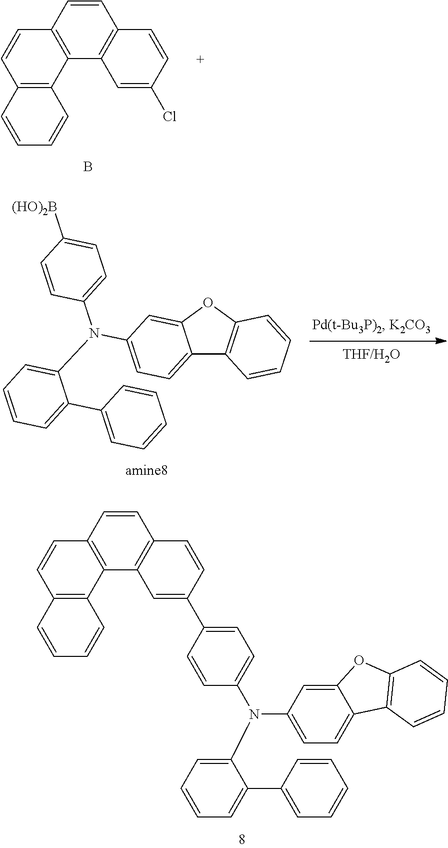

Compound B (15 g, 57.1 mmol) and Compound amine8 (27.3 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 12 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 23.6 g of Compound 8. (Yield: 65%, MS: [M+H]⁺=638)

### Preparation Example 9

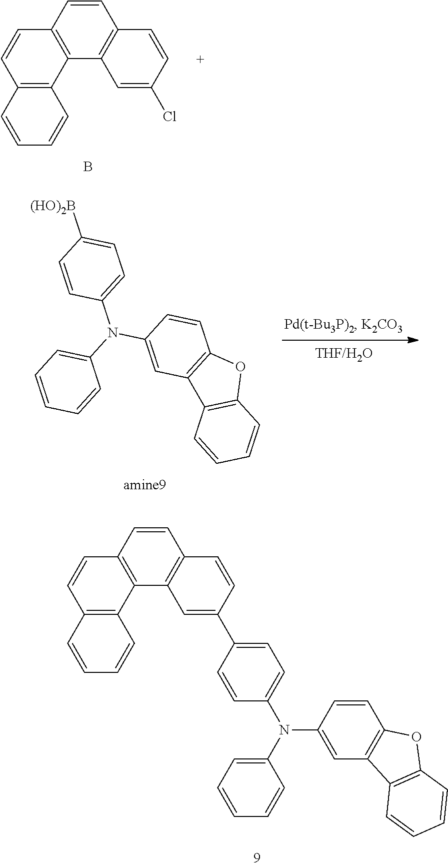

Compound B (15 g, 57.1 mmol) and Compound amine9 (22.7 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 11 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 21.8 g of Compound 9. (Yield: 68%, MS: [M+H]⁺=562)

### Preparation Example 10

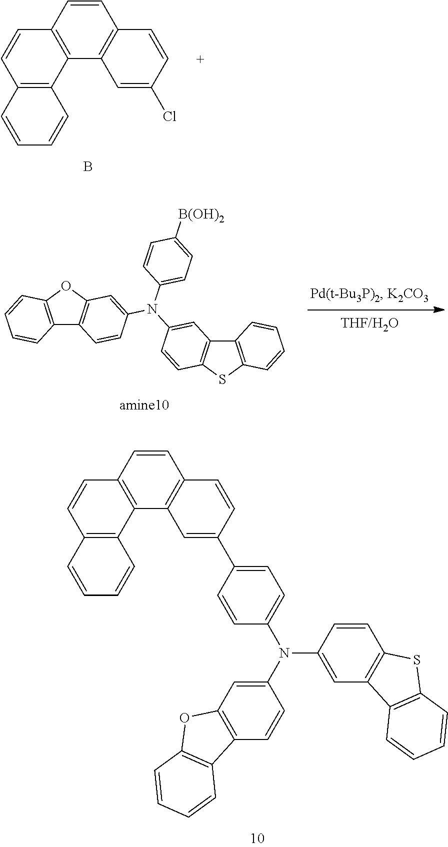

Compound B (15 g, 57.1 mmol) and Compound amine10 (29.1 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 8 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 27 g of Compound 10. (Yield: 71%, MS: [M+H]⁺=668)

### Preparation Example 11

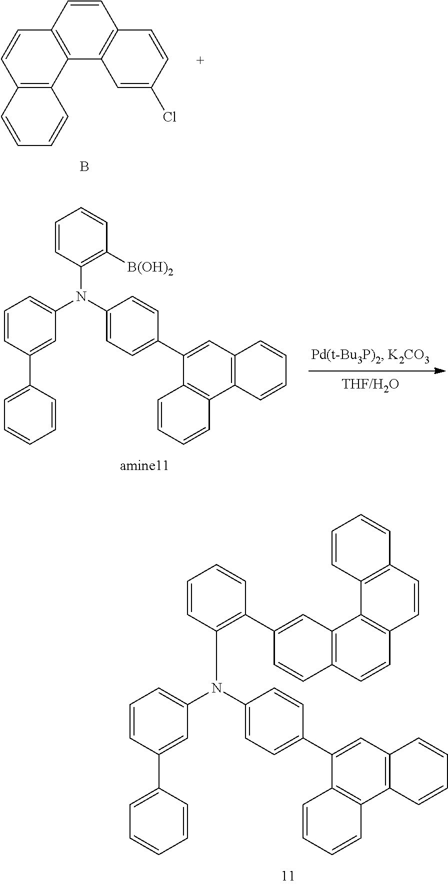

Compound B (15 g, 57.1 mmol) and Compound amine11 (32.5 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 12 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 33 g of Compound 11. (Yield: 80%, MS: [M+H]⁺=724)

### Preparation Example 12

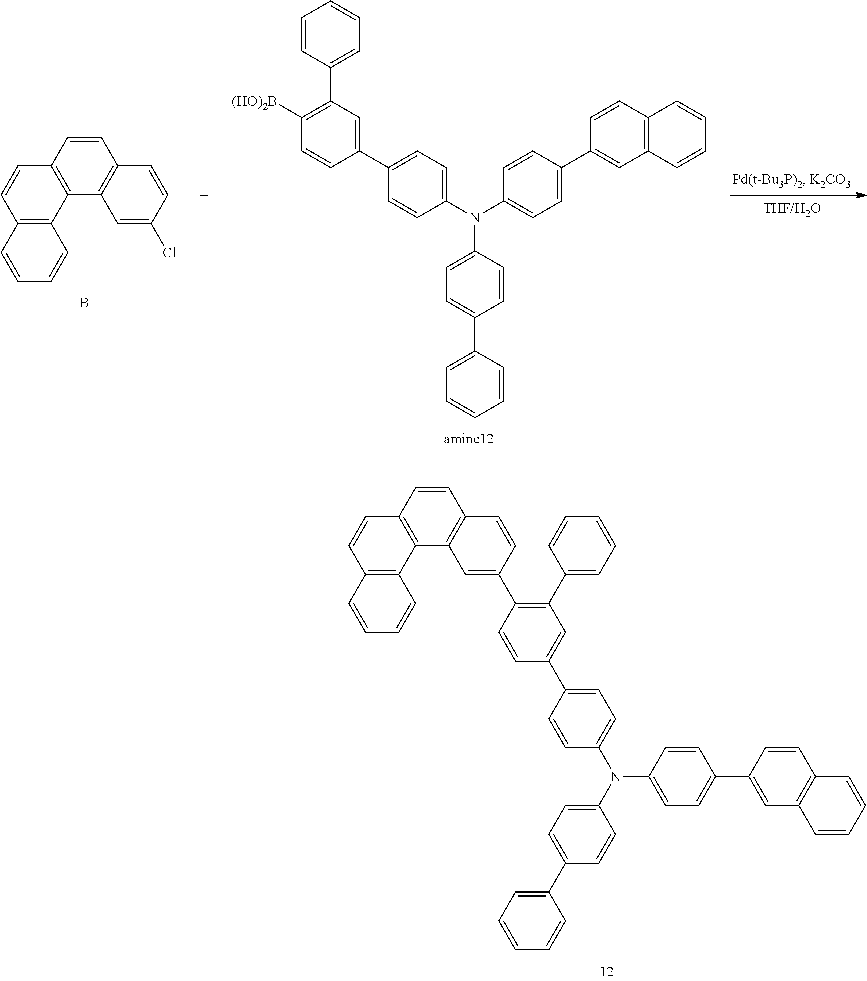

Compound B (15 g, 57.1 mmol) and Compound amine12 (38.6 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 8 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 33.5 g of Compound 12. (Yield: 71%, MS: [M+H]⁺=826)

### Preparation Example 13

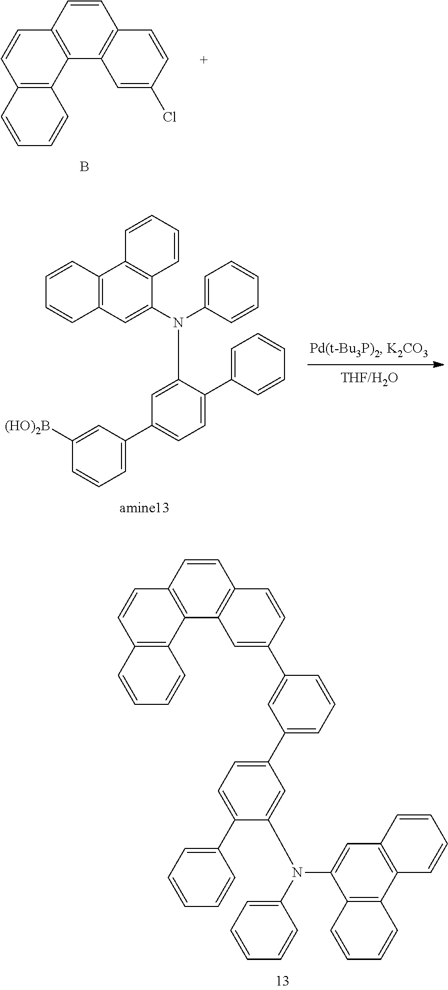

Compound B (15 g, 57.1 mmol) and Compound amine13 (32.5 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 9 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 27.3 g of Compound 13. (Yield: 66%, MS: [M+H]⁺=724)

### Preparation Example 14

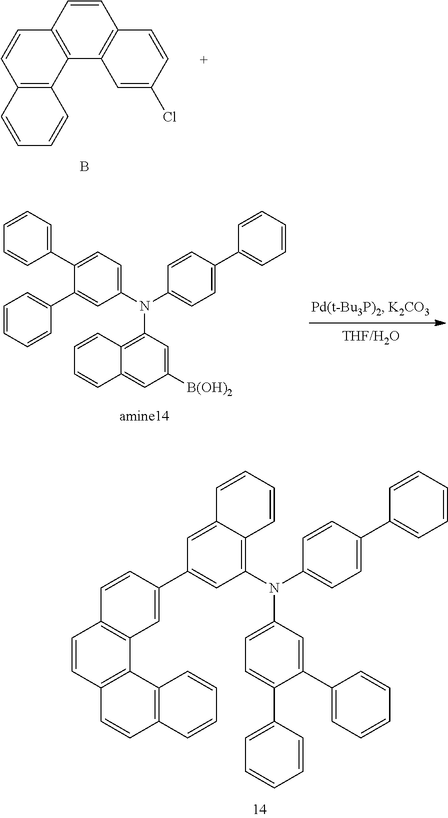

Compound B (15 g, 57.1 mmol) and Compound amine14 (34 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 8 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 30.8 g of Compound 14. (Yield: 72%, MS: [M+H]⁺=750)

### Preparation Example 15

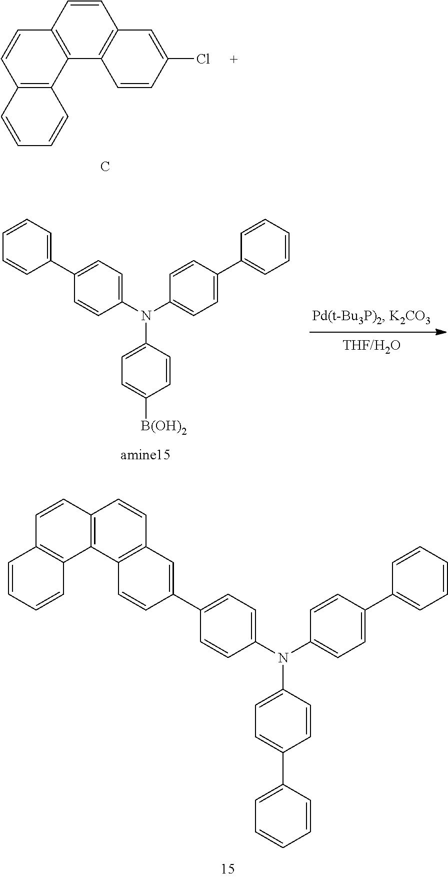

Compound C (15 g, 57.1 mmol) and Compound amine15 (26.5 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 11 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 28.5 g of Compound 15. (Yield: 80%, MS: [M+H]⁺=624)

### Preparation Example 16

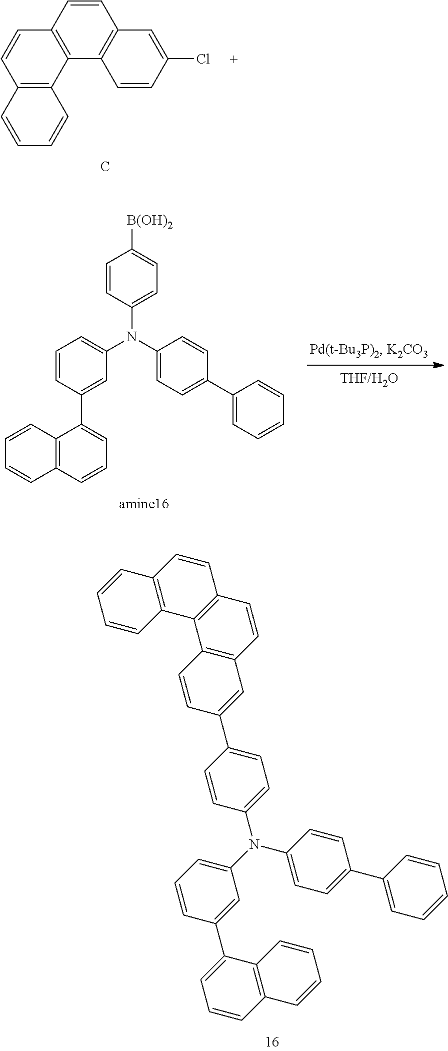

Compound C (15 g, 57.1 mmol) and Compound amine16 (29.5 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 8 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 23.8 g of Compound 16. (Yield: 62%, MS: [M+H]⁺=674)

### Preparation Example 17

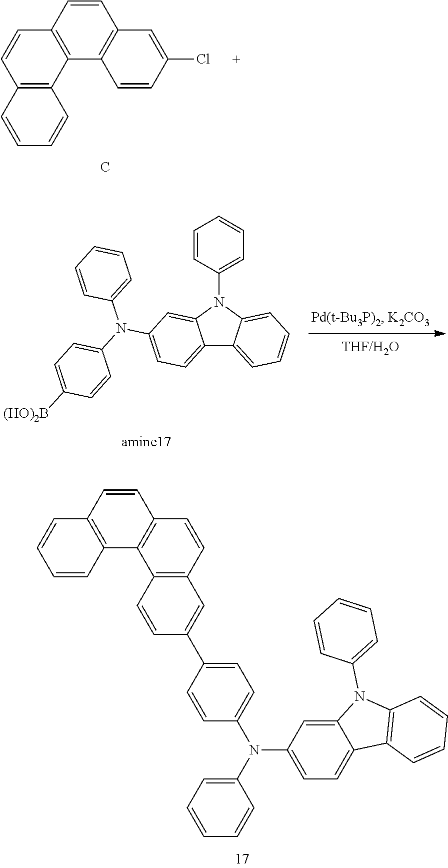

Compound C (15 g, 57.1 mmol) and Compound amine17 (27.2 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 9 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 24.7 g of Compound 17. (Yield: 68%, MS: [M+H]⁺=637)

### Preparation Example 18

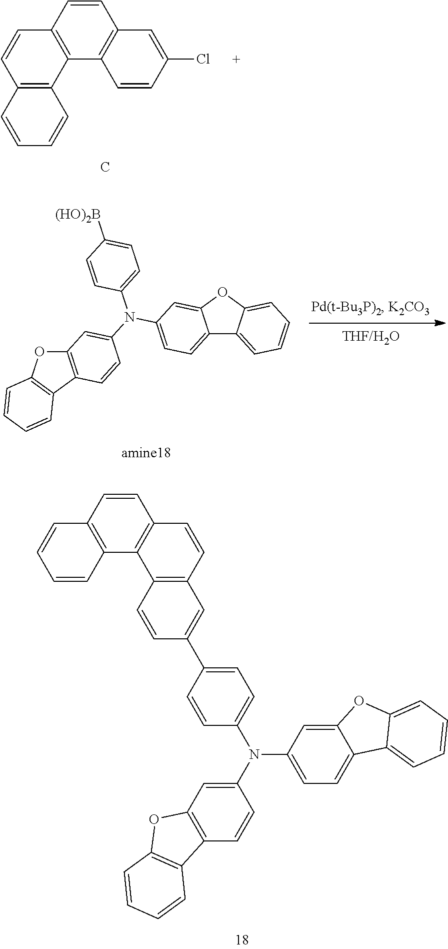

Compound C (15 g, 57.1 mmol) and Compound amine18 (28.1 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 10 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 28.3 g of Compound 18. (Yield: 76%, MS: [M+H]⁺=652)

### Preparation Example 19

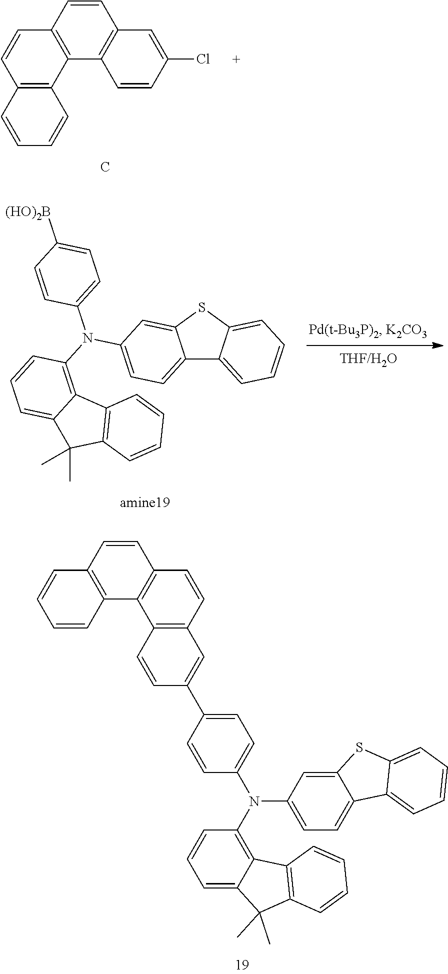

Compound C (15 g, 57.1 mmol) and Compound amine19 (30.7 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 12 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 30.5 g of Compound 19. (Yield: 77%, MS: [M+H]⁺=694)

### Preparation Example 20

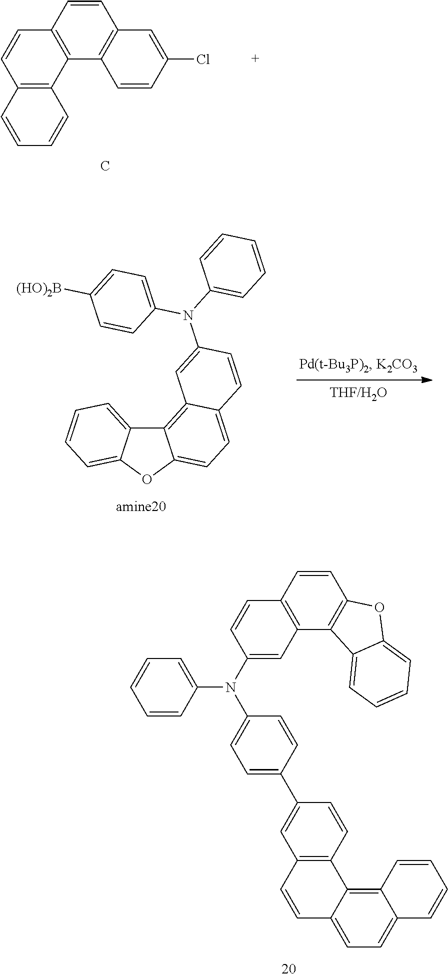

Compound C (15 g, 57.1 mmol) and Compound amine20 (25.7 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 10 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 24.4 g of Compound 20. (Yield: 70%, MS: [M+H]⁺=612)

### Preparation Example 21

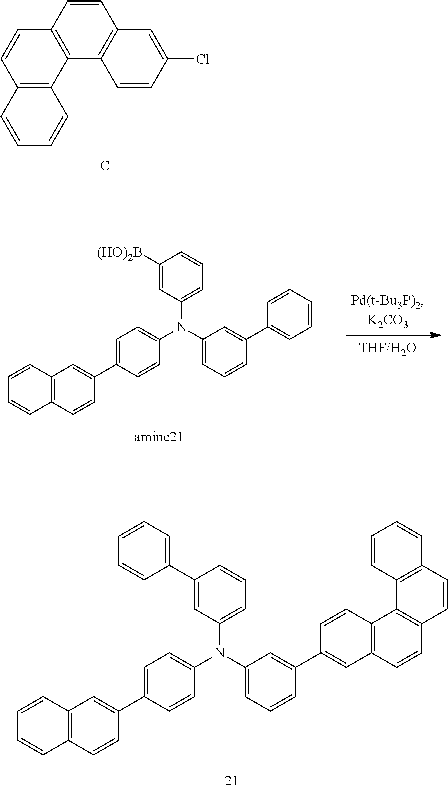

Compound C (15 g, 57.1 mmol) and Compound amine21 (29.5 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 10 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 25.4 g of Compound 21. (Yield: 66%, MS: [M+H]⁺=674)

### Preparation Example 22

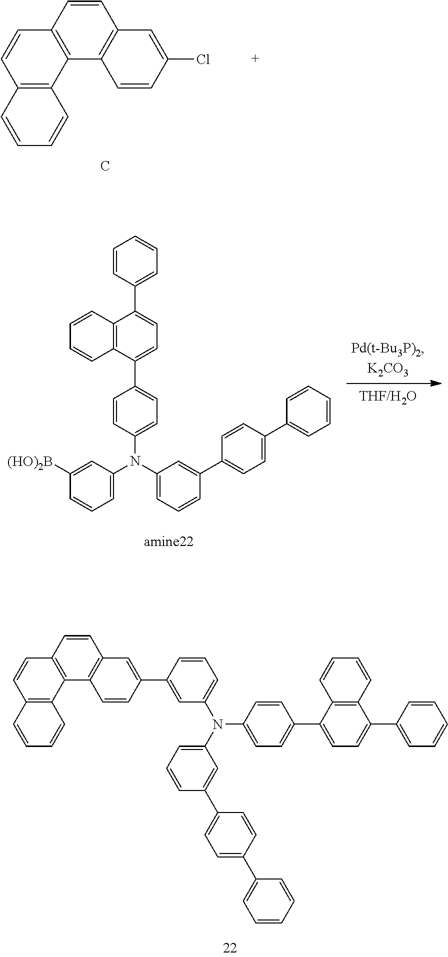

Compound C (15 g, 57.1 mmol) and Compound amine22 (38.6 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 9 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 30.2 g of Compound 22. (Yield: 64%, MS: [M+H]⁺=826)

### Preparation Example 23

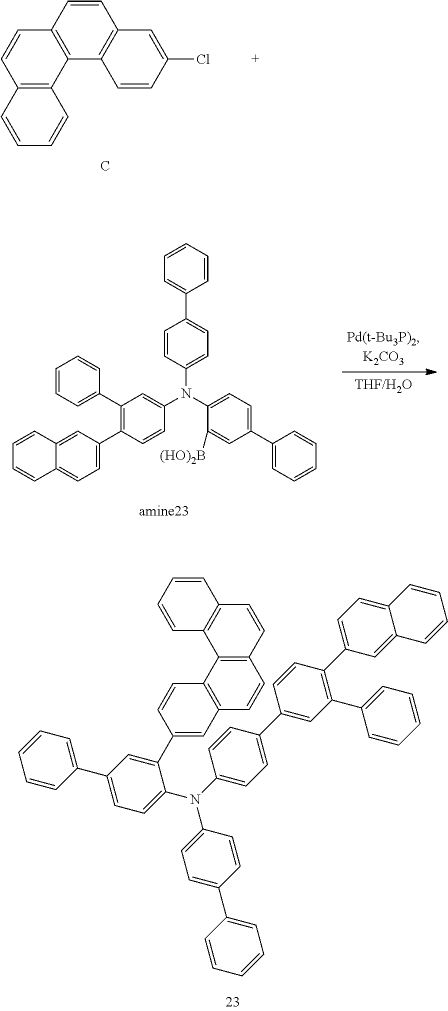

Compound C (15 g, 57.1 mmol) and Compound amine23 (38.6 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 8 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 33.9 g of Compound 23. (Yield: 72%, MS: [M+H]⁺=826)

### Preparation Example 24

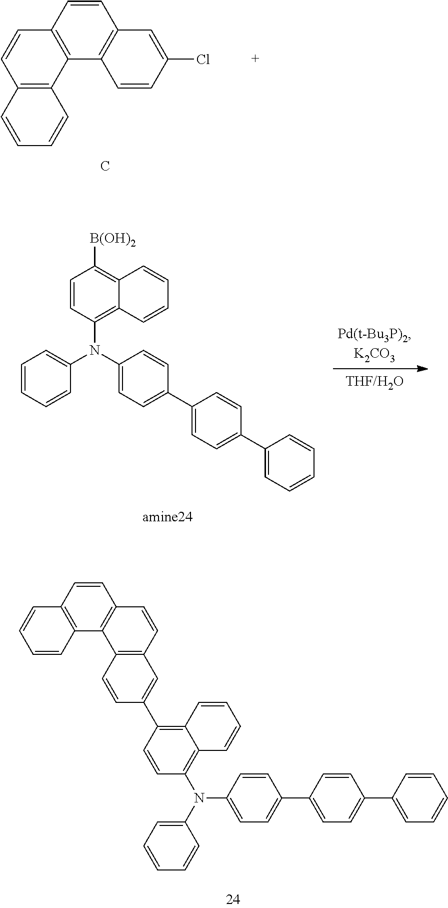

Compound C (15 g, 57.1 mmol) and Compound amine24 (29.5 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 8 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 23.8 g of Compound 24. (Yield: 62%, MS: [M+H]⁺=674)

### Preparation Example 25

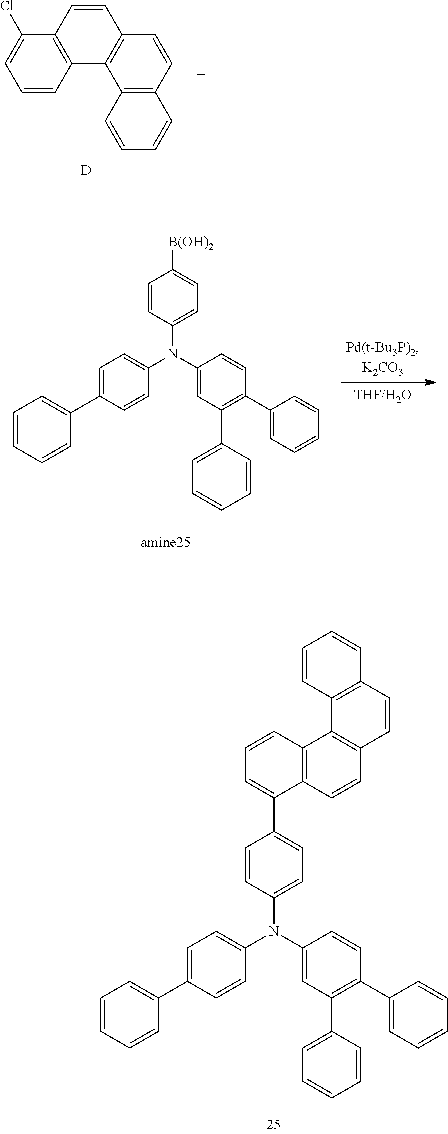

Compound D (15 g, 57.1 mmol) and Compound amine25 (31 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 12 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 29.5 g of Compound 25. (Yield: 74%, MS: [M+H]⁺=700)

### Preparation Example 26

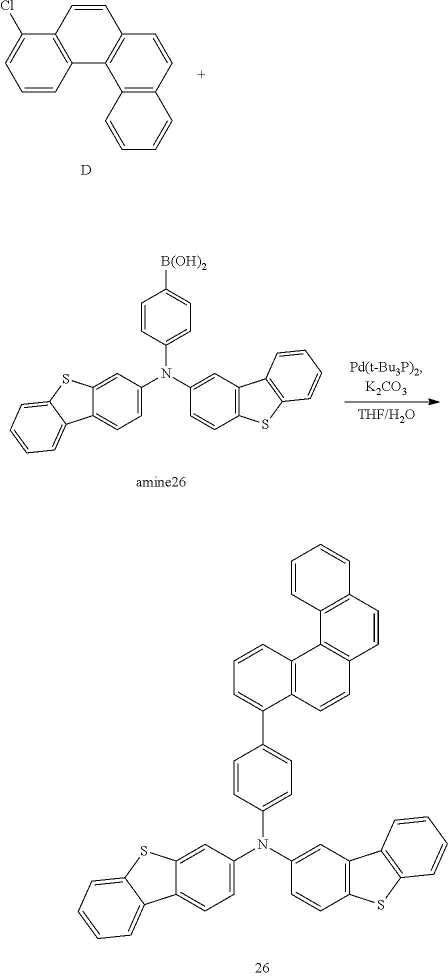

Compound D (15 g, 57.1 mmol) and Compound amine26 (30.1 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 9 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 25.4 g of Compound 26. (Yield: 65%, MS: [M+H]⁺=684)

### Preparation Example 27

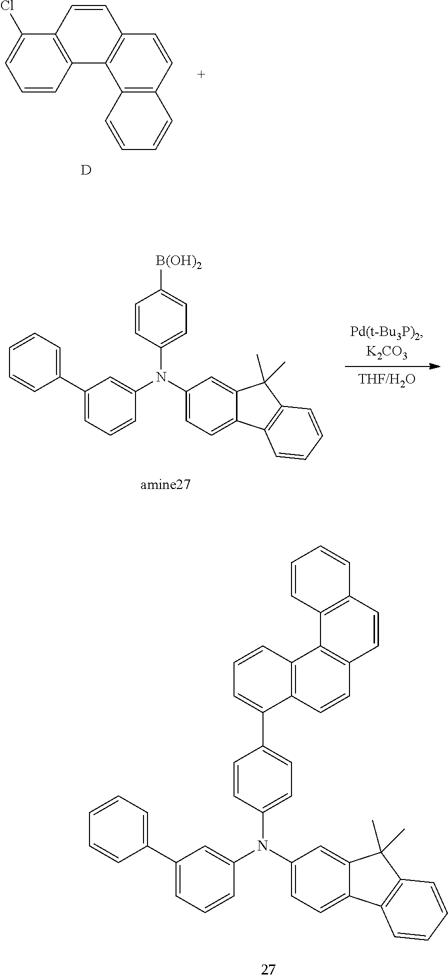

Compound D (15 g, 57.1 mmol) and Compound amine27 (28.9 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 10 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 29.2 g of Compound 27. (Yield: 77%, MS: [M+H]⁺=664)

### Preparation Example 28

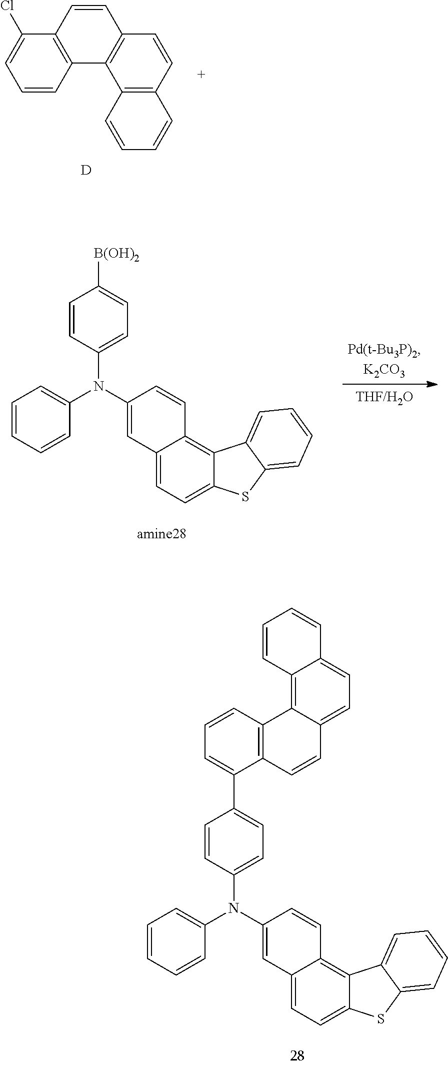

Compound D (15 g, 57.1 mmol) and Compound amine28 (26.7 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 11 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 21.8 g of Compound 28. (Yield: 61%, MS: [M+H]⁺=628)

### Preparation Example 29

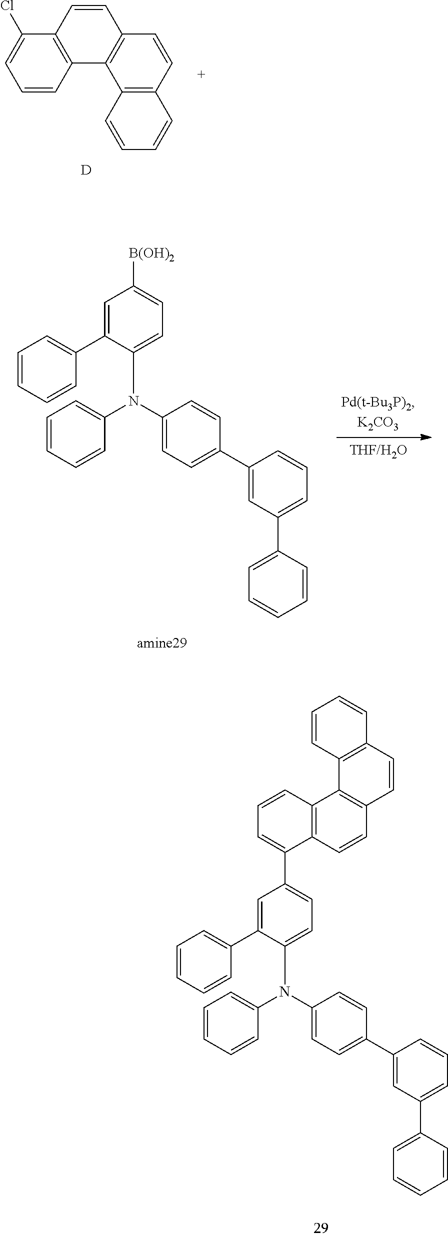

Compound D (15 g, 57.1 mmol) and Compound amine29 (31 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 8 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 31.9 g of Compound 29. (Yield: 80%, MS: [M+H]⁺=700)

### Preparation Example 30

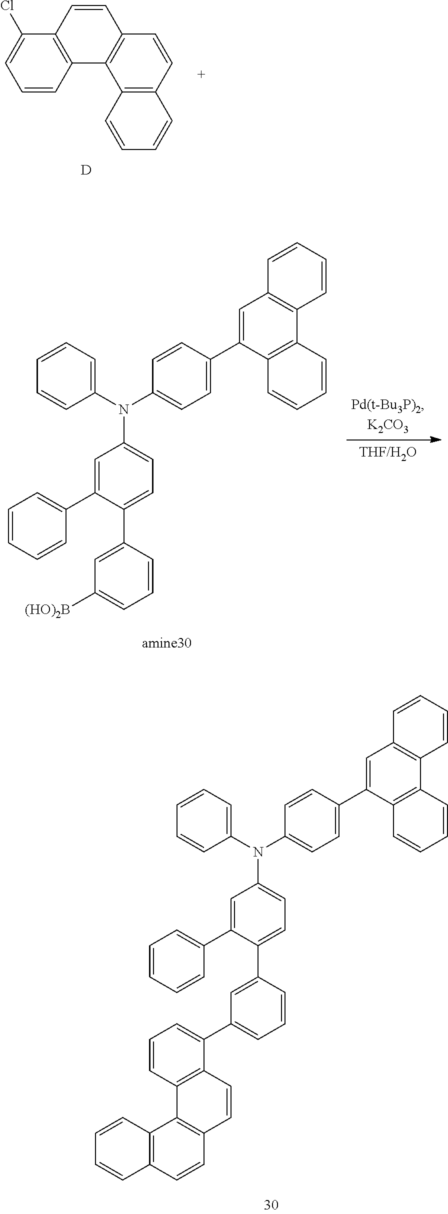

Compound D (15 g, 57.1 mmol) and Compound amine30 (37 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 10 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 32.4 g of Compound 30. (Yield: 71%, MS: [M+H]⁺=800)

### Preparation Example 31

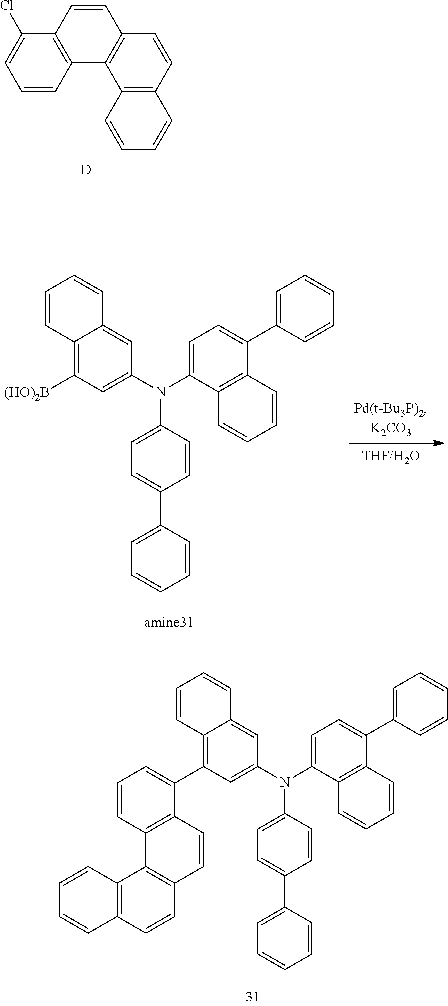

Compound D (15 g, 57.1 mmol) and Compound amine31 (32.5 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 10 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 29.3 g of Compound 31. (Yield: 71%, MS: [M+H]⁺=724)

### Preparation Example 32

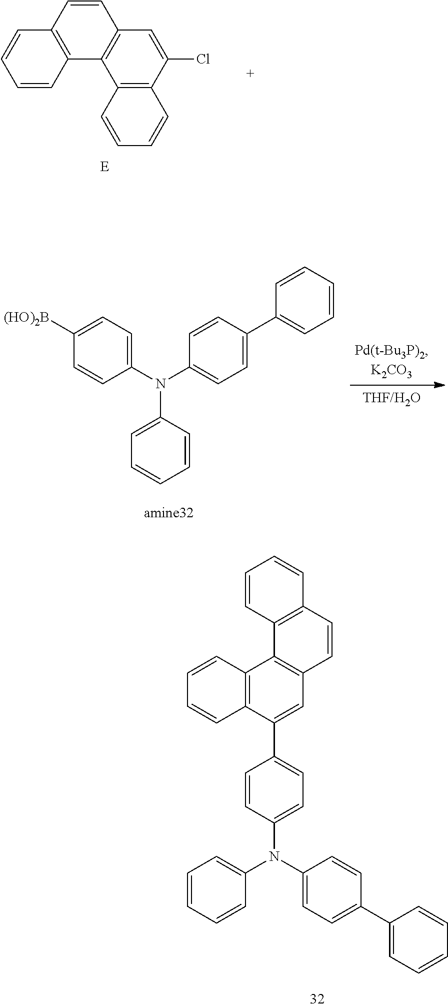

Compound E (15 g, 57.1 mmol) and Compound amine32 (21.9 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 8 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 19.1 g of Compound 32. (Yield: 61%, MS: [M+H]⁺=548)

### Preparation Example 33

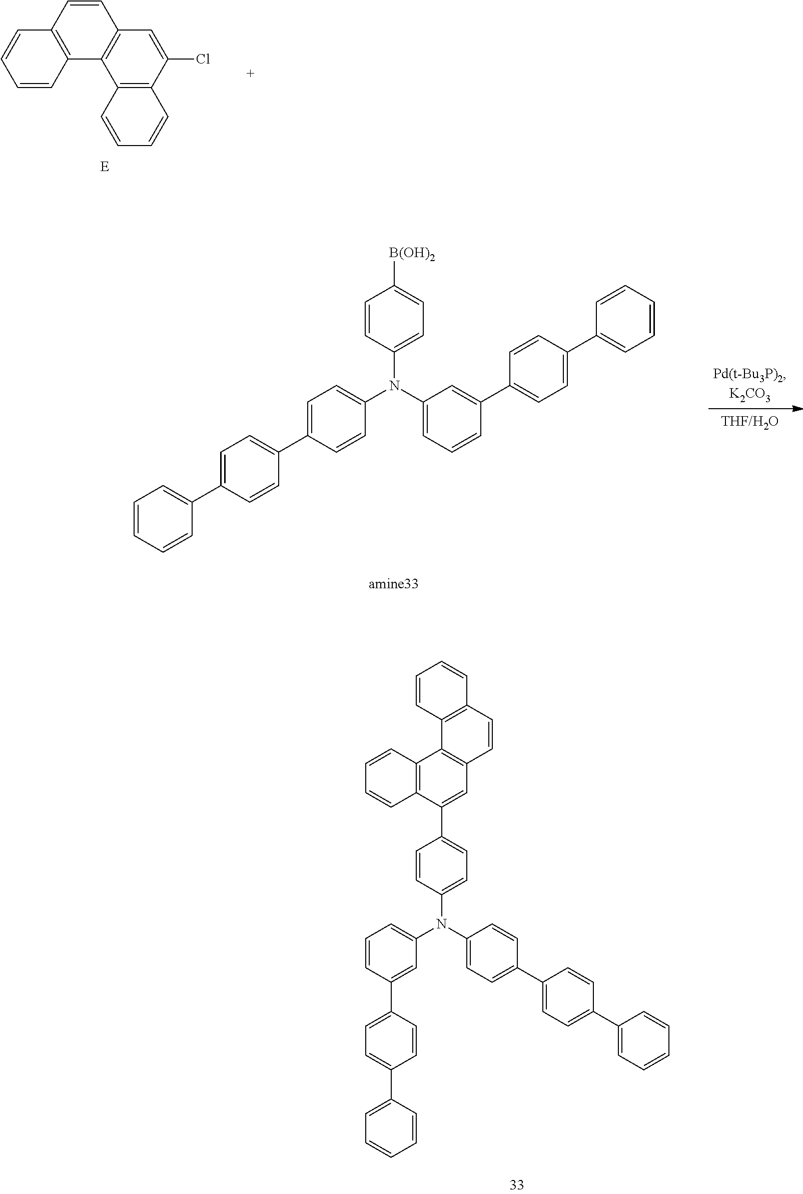

Compound E (15 g, 57.1 mmol) and Compound amine33 (35.6 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(O) (0.3 g, 0.6 mmol) was added. After reacting for 12 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 31.9 g of Compound 33. (Yield: 72%, MS: [M+H]⁺=776)

### Preparation Example 34

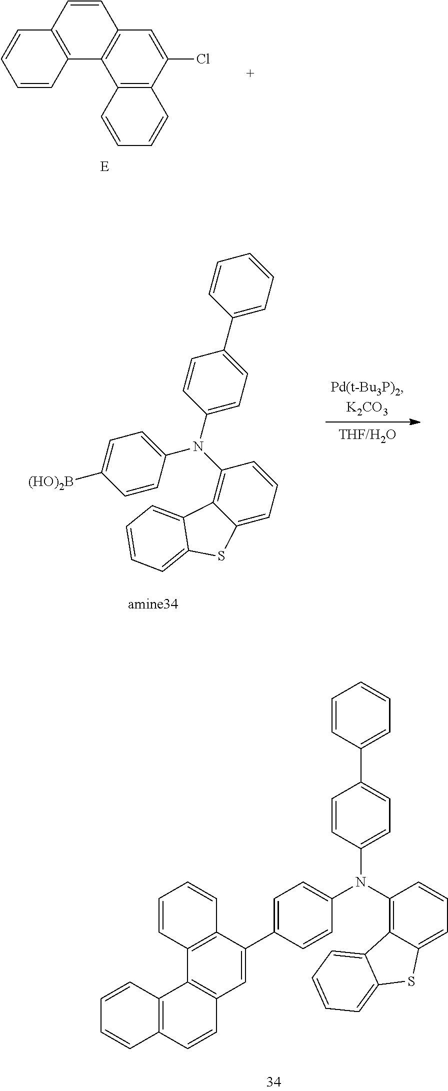

Compound E (15 g, 57.1 mmol) and Compound amine34 (28.3 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 8 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 25 g of Compound 34. (Yield: 67%, MS: [M+H]⁺=654)

### Preparation Example 35

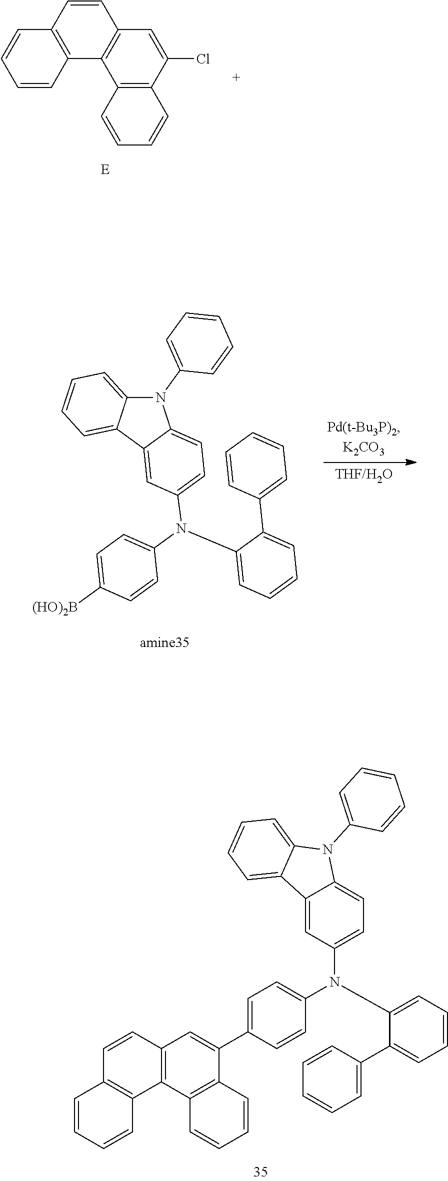 [SMILES: Clc1cc2ccc3ccccc3c2c2ccccc12 ; OB(O)c1ccc(N(c2ccc3c(c2)c2ccccc2n3-c2ccccc2)c2ccccc2-c2ccccc2)cc1 ; c1ccc(-c2ccccc2N(c2ccc(-c3cc4ccc5ccccc5c4c4ccccc34)cc2)c2ccc3c(c2)c2ccccc2n3-c2ccccc2)cc1]

Compound E (15 g, 57.1 mmol) and Compound amine35 (31.8 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 12 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 26.4 g of Compound 35. (Yield: 65%, MS: [M+H]⁺=713)

### Preparation Example 36

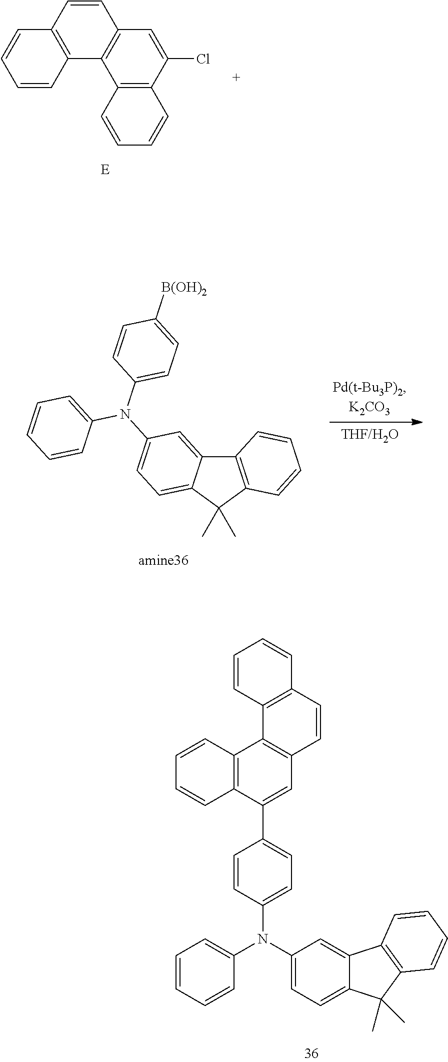 [SMILES: Clc1cc2ccc3ccccc3c2c2ccccc12 ; CC1(C)c2ccccc2-c2cc(N(c3ccccc3)c3ccc(B(O)O)cc3)ccc21 ; CC1(C)c2ccccc2-c2cc(N(c3ccccc3)c3ccc(-c4cc5ccc6ccccc6c5c5ccccc45)cc3)ccc21]

Compound E (15 g, 57.1 mmol) and Compound amine36 (24.3 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 11 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 21.1 g of Compound 36. (Yield: 63%, MS: [M+H]⁺=588)

### Preparation Example 37

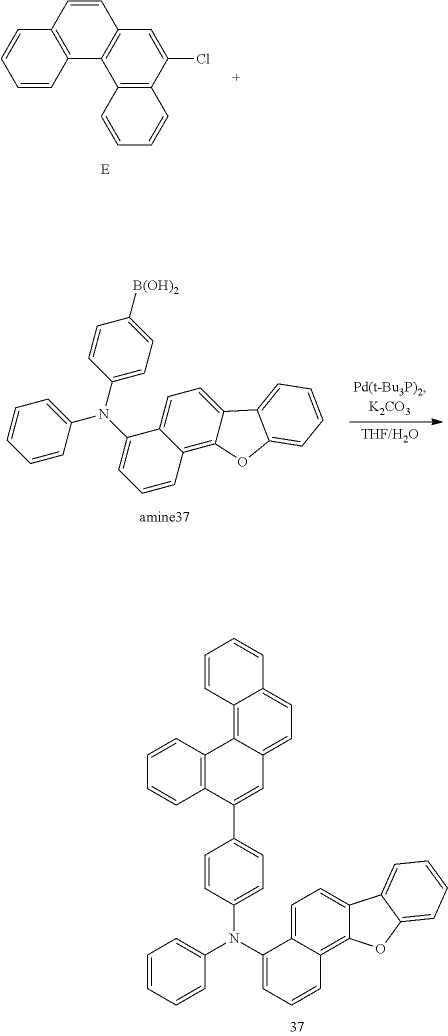 [SMILES: Clc1cc2ccc3ccccc3c2c2ccccc12 ; OB(O)c1ccc(N(c2ccccc2)c2cccc3c2ccc2c4ccccc4oc32)cc1 ; c1ccc(N(c2ccc(-c3cc4ccc5ccccc5c4c4ccccc34)cc2)c2cccc3c2ccc2c4ccccc4oc32)cc1]

Compound E (15 g, 57.1 mmol) and Compound amine37 (25.7 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 11 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 25.5 g of Compound 37. (Yield: 73%, MS: [M+H]⁺=612)

### Preparation Example 38

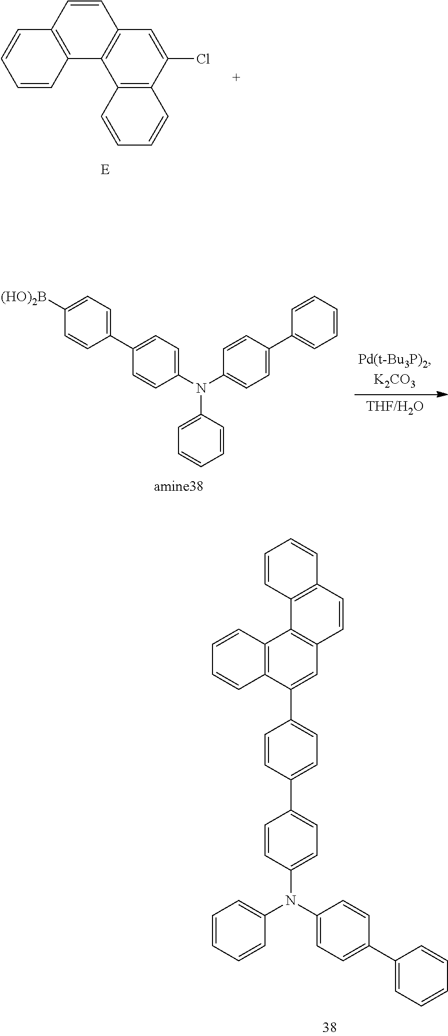 [SMILES: Clc1cc2ccc3ccccc3c2c2ccccc12 ; OB(O)c1ccc(-c2ccc(N(c3ccccc3)c3ccc(-c4ccccc4)cc3)cc2)cc1 ; c1ccc(-c2ccc(N(c3ccccc3)c3ccc(-c4ccc(-c5cc6ccc7ccccc7c6c6ccccc56)cc4)cc3)cc2)cc1]

Compound E (15 g, 57.1 mmol) and Compound amine38 (26.5 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 12 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 23.1 g of Compound 38. (Yield: 65%, MS: [M+H]⁺=624)

### Preparation Example 39

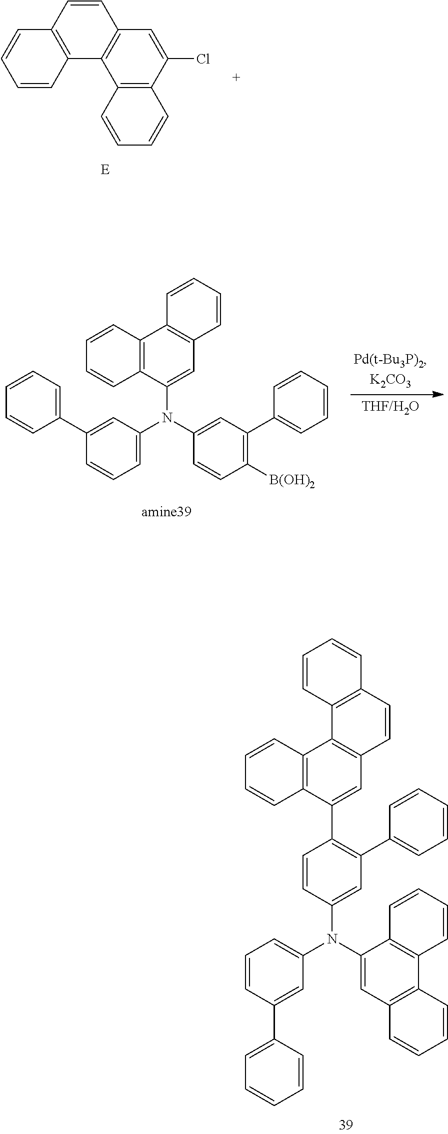 [SMILES: Clc1cc2ccc3ccccc3c2c2ccccc12 ; OB(O)c1ccc(N(c2cccc(-c3ccccc3)c2)c2cc3ccccc3c3ccccc23)cc1-c1ccccc1 ; c1ccc(-c2cccc(N(c3ccc(-c4cc5ccc6ccccc6c5c5ccccc45)c(-c4ccccc4)c3)c3cc4ccccc4c4ccccc34)c2)cc1]

Compound E (15 g, 57.1 mmol) and Compound amine39 (32.5 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 11 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 25.2 g of Compound 39. (Yield: 61%, MS: [M+H]⁺=724)

### Preparation Example 40

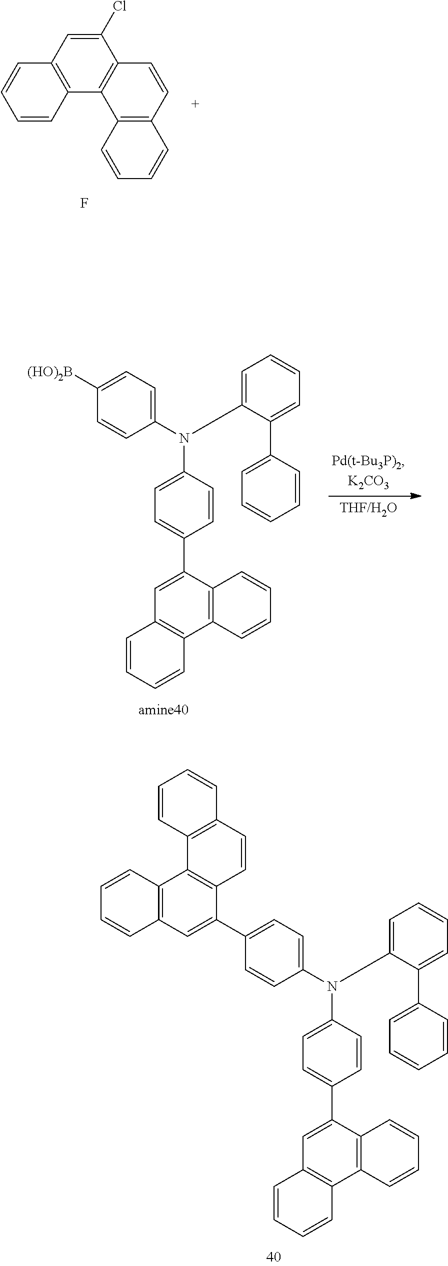

Compound F (15 g, 57.1 mmol) and Compound amine40 (32.5 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 11 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 26 g of Compound 40. (Yield: 63%, MS: [M+H]⁺=724)

### Preparation Example 41

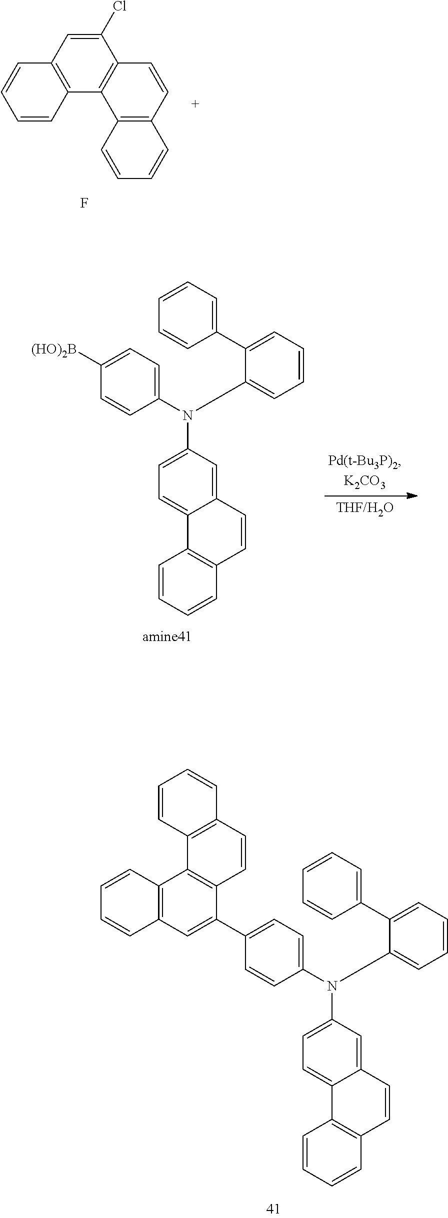

Compound F (15 g, 57.1 mmol) and Compound amine41 (27.9 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 11 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 26.6 g of Compound 41. (Yield: 72%, MS: [M+H]⁺=648)

### Preparation Example 42

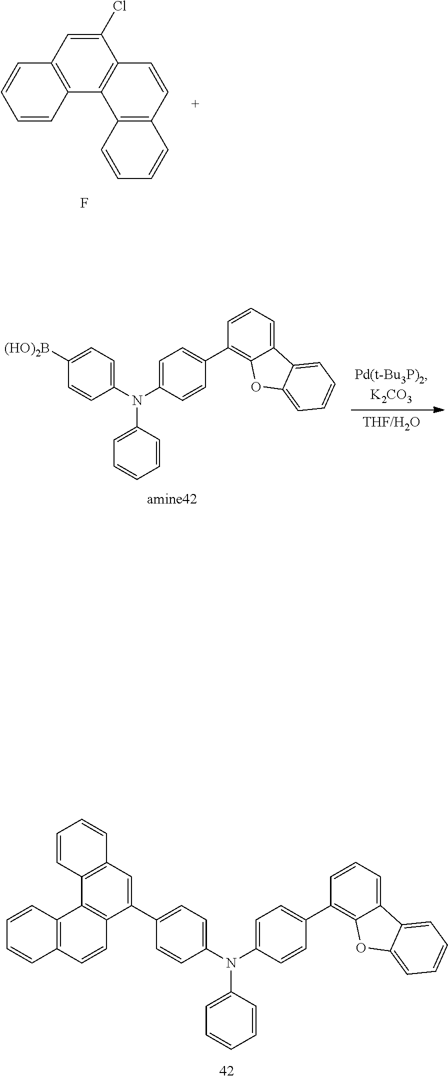

Compound F (15 g, 57.1 mmol) and Compound amine42 (27.3 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 10 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 29.1 g of Compound 42. (Yield: 80%, MS: [M+H]⁺=638)

### Preparation Example 43

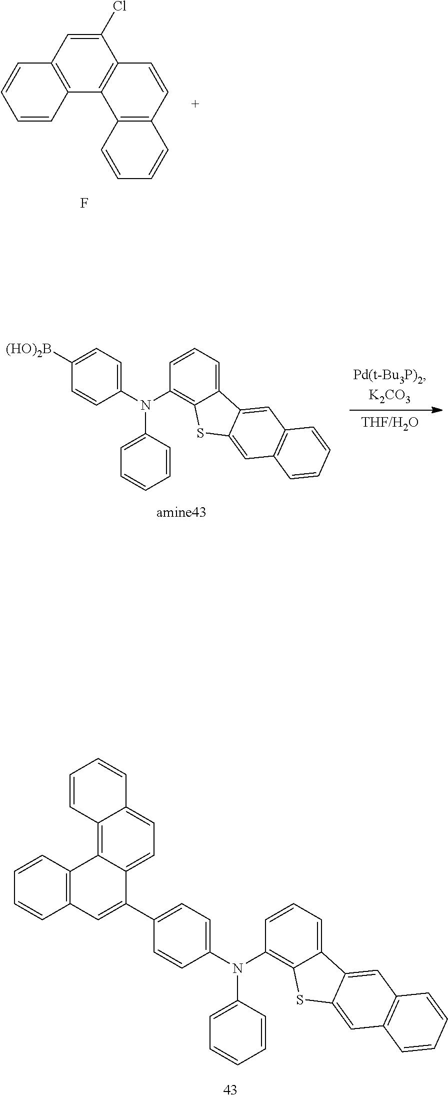

Compound F (15 g, 57.1 mmol) and Compound amine43 (26.7 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 8 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 23.3 g of Compound 43. (Yield: 65%, MS: [M+H]⁺=628)

### Preparation Example 44

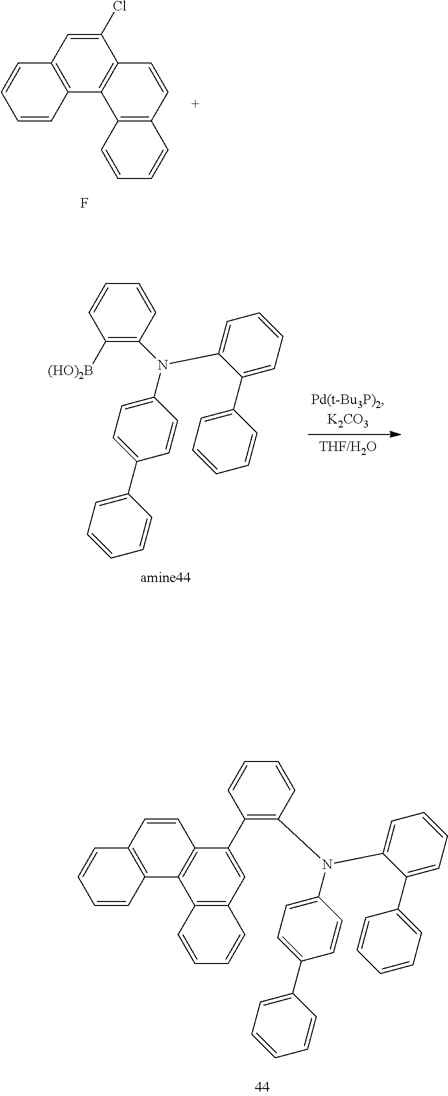

Compound F (15 g, 57.1 mmol) and Compound amine44 (26.5 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 10 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 26 g of Compound 44. (Yield: 73%, MS: [M+H]⁺=624)

### Preparation Example 45

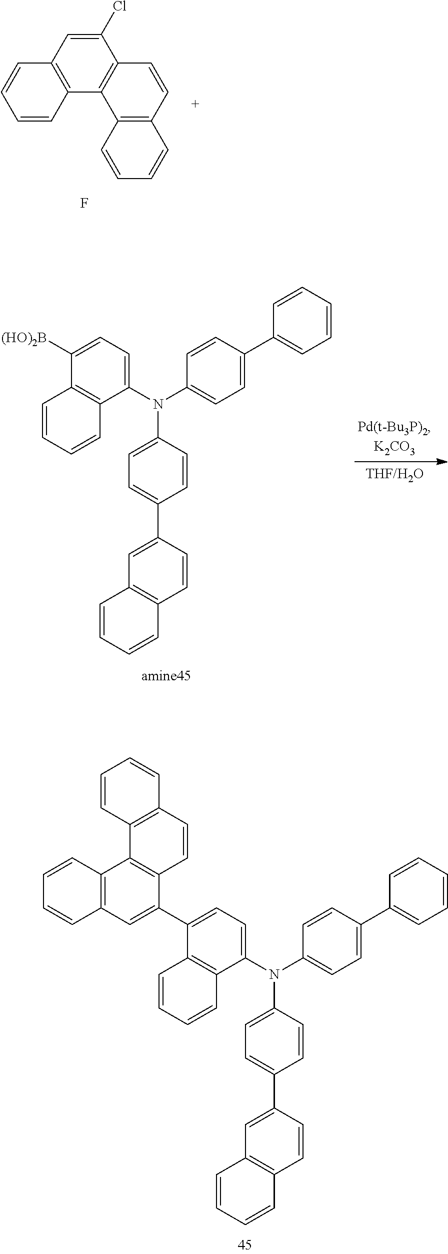

Compound F (15 g, 57.1 mmol) and Compound amine45 (32.5 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 12 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 30.6 g of Compound 45. (Yield: 74%, MS: [M+H]⁺=724)

### Preparation Example 46

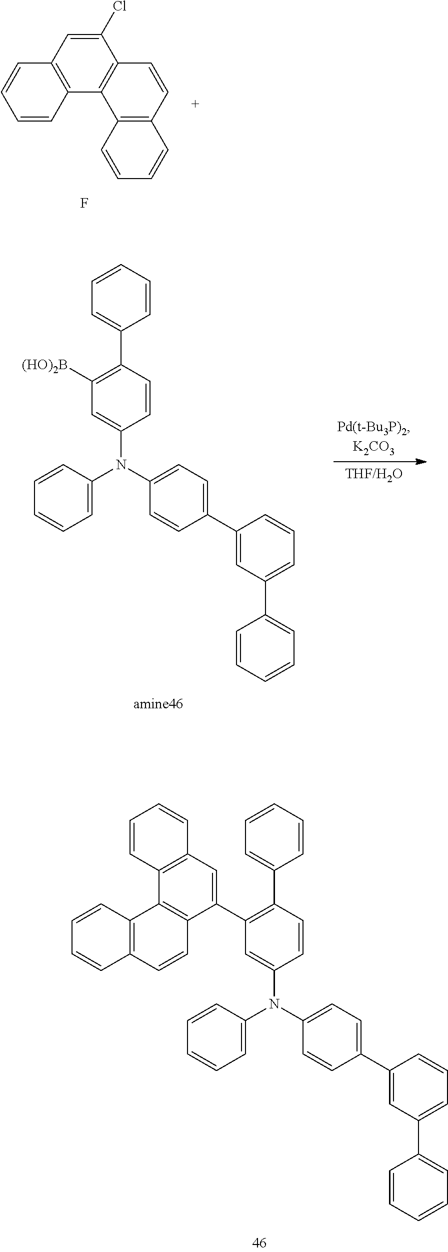

Compound F (15 g, 57.1 mmol) and Compound amine46 (31 g, 59.9 mmol) were added to 300 ml of THF under a nitrogen atmosphere, and the mixture was stirred and refluxed. Then, potassium carbonate (23.7 g, 171.3 mmol) was dissolved in 71 ml of water and added thereto, and the mixture was sufficiently stirred and then bis(tri-tert-butylphosphine)palladium(0) (0.3 g, 0.6 mmol) was added. After reacting for 8 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 25.6 g of Compound 46. (Yield: 64%, MS: [M+H]⁺=700)

### EXAMPLE

### Example 1-1

A glass substrate on which a thin film of ITO (indium tin oxide) was coated in a thickness of 1,400 Å was put into distilled water containing a detergent dissolved therein and ultrasonically washed. In this case, the detergent used was a product commercially available from Fischer Co. and the distilled water was one which had been twice filtered by using a filter commercially available from Millipore Co. The ITO was washed for 30 minutes, and ultrasonic washing was then repeated twice for 10 minutes using distilled water. After the washing with distilled water was completed, the substrate was ultrasonically washed with isopropyl alcohol, acetone, and methanol solvent, and dried, after which it was transported to a plasma cleaner. Then, the substrate was cleaned with oxygen plasma for 5 minutes, and then transferred to a vacuum evaporator.

On the ITO transparent electrode thus prepared, the following compound HI-1 was formed in a thickness of 1150 Å as a hole injection layer, but the following compound A-1 was p-doped at a concentration of 1.5 wt. %. The following compound HT-1 was vacuum deposited on the hole injection layer to form a hole transport layer with a film thickness of 800 Å. Then, the compound 1 prepared in Preparation Example 1 was thermally vacuum deposited to a thickness of 150 Å as an electron blocking layer. Then, the following compound BH and the following compound BD were vacuum-deposited to a thickness of 200 Å in a weight ratio of 25:1 as a light emitting layer. Then, the following compound HB-1 was vacuum-deposited to a thickness of 50 Å as a hole blocking layer. Then, the following compound ET-1 and the following compound LiQ were thermally vacuum-deposited to a thickness of 310 Å in a weight ratio of 1:1 as a layer for simultaneously performing electron transport and electron injection. Lithium fluoride (LiF) and aluminum were sequentially deposited to have a thickness of 12 Å and 1,000 Å, respectively, on the electron injection and transport layer, to form a cathode, thereby manufacturing an organic light emitting device.

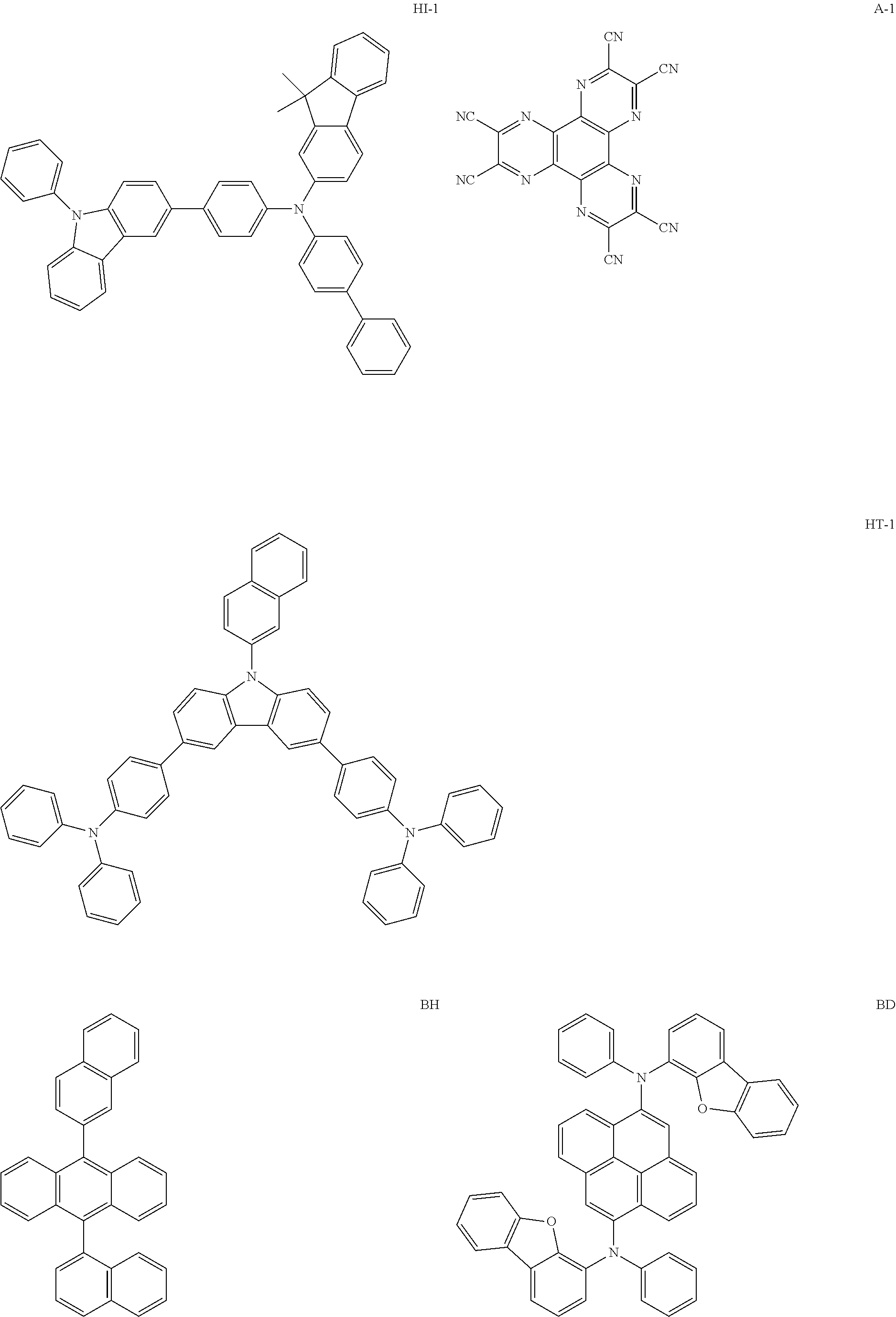

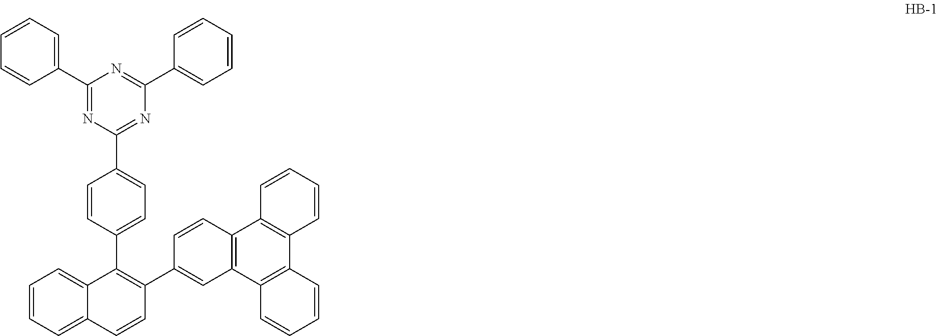 [SMILES: c1ccc(-c2nc(-c3ccccc3)nc(-c3ccc(-c4c(-c5ccc6c7ccccc7c7ccccc7c6c5)ccc5ccccc45)cc3)n2)cc1]

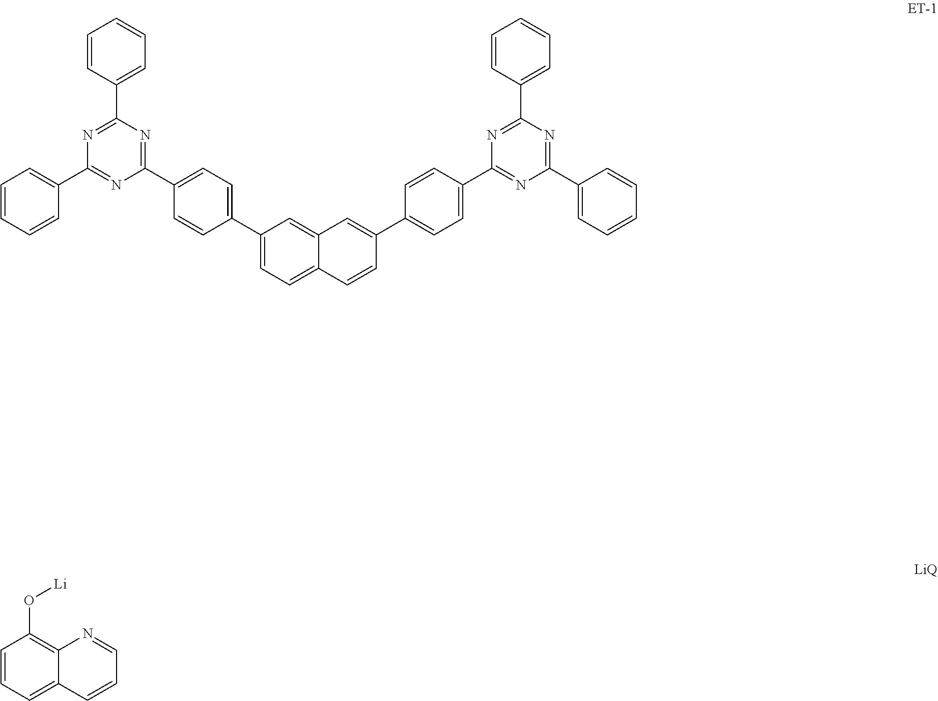

In the above-mentioned processes, the deposition rates of the organic materials were maintained at 0.4˜0.7 Å/sec, the deposition rates of lithium fluoride and the aluminum of the cathode were maintained at 0.3 Å/see and 2 Å/see, respectively, and the degree of vacuum during the deposition was maintained at 2×10⁻⁷ to 5×10⁻⁶ torr, thereby manufacturing an organic light emitting device.

### Examples 1-2 to Example 1-46

The organic light emitting devices of Examples 1-2 to 1-46 were manufactured in the same manner as in Example 1-1, except that the compounds shown in Table 1 below were used instead of Compound 1.

### Comparative Example 1-1 to Comparative Example 1-9

The organic light emitting devices of Comparative Example 1-1 to 1-9 were manufactured in the same manner as in Example 1-1, except that the following compounds EB1 to EB9 were used instead of Compound 1.

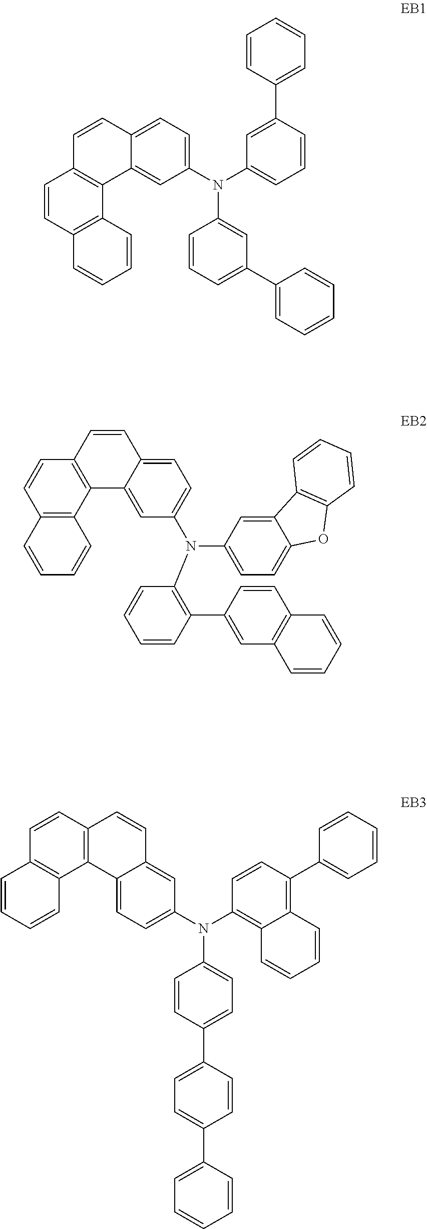 [SMILES: c1ccc(-c2cccc(N(c3cccc(-c4ccccc4)c3)c3ccc4ccc5ccc6ccccc6c5c4c3)c2)cc1 ; c1ccc(N(c2ccc3oc4ccccc4c3c2)c2ccc3ccc4ccc5ccccc5c4c3c2)c(-c2ccc3ccccc3c2)c1 ; c1ccc(-c2ccc(-c3ccc(N(c4ccc5c(ccc6ccc7ccccc7c65)c4)c4ccc(-c5ccccc5)c5ccccc45)cc3)cc2)cc1]

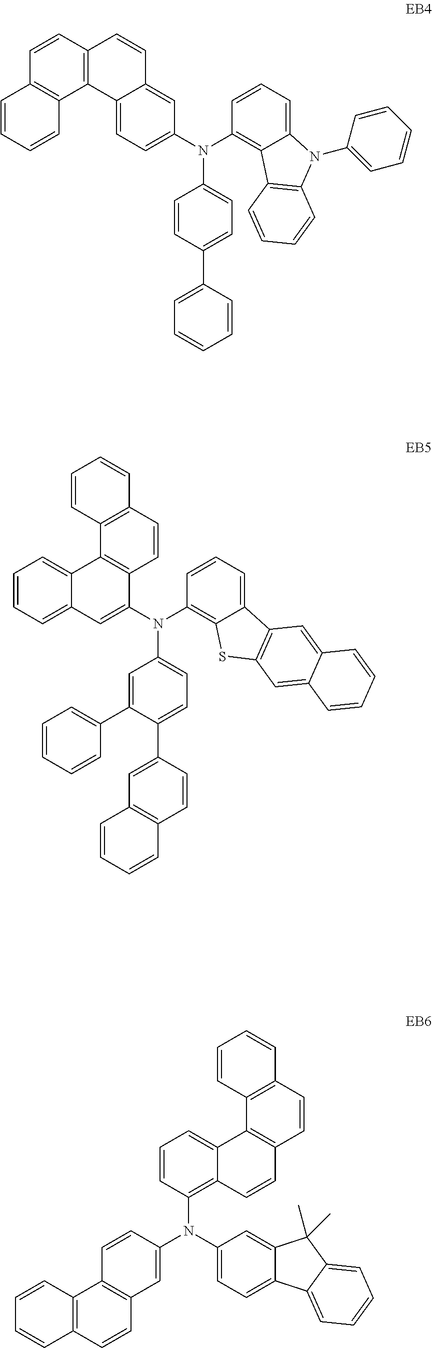 [SMILES: c1ccc(-c2ccc(N(c3ccc4c(ccc5ccc6ccccc6c54)c3)c3cccc4c3c3ccccc3n4-c3ccccc3)cc2)cc1 ; c1ccc(-c2cc(N(c3cc4ccccc4c4c3ccc3ccccc34)c3cccc4c3sc3cc5ccccc5cc34)ccc2-c2ccc3ccccc3c2)cc1 ; CC1(C)c2ccccc2-c2ccc(N(c3ccc4c(ccc5ccccc54)c3)c3cccc4c3ccc3ccc5ccccc5c34)cc21]

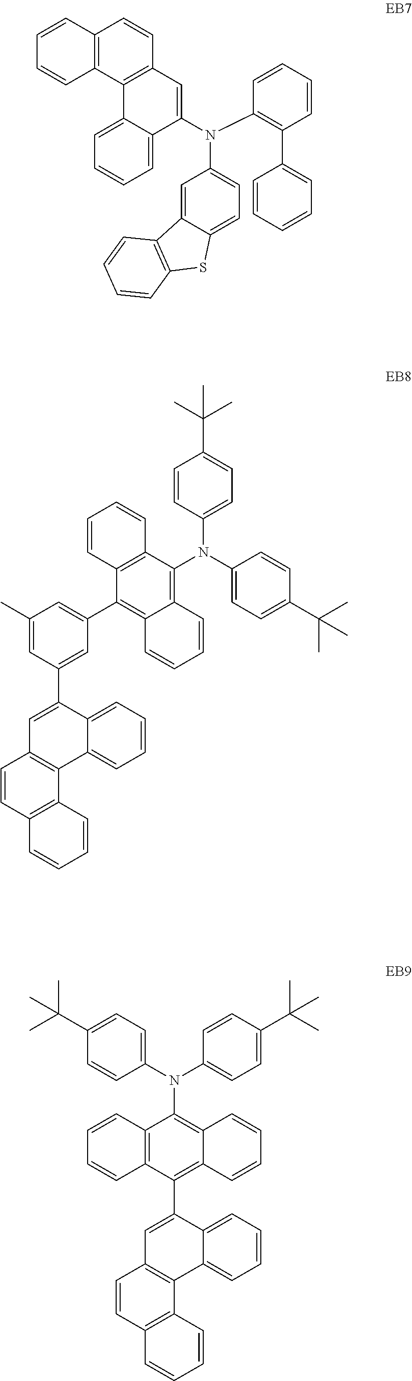

### Experimental Example

The driving voltage and efficiency were measured by applying a current (10 mA/cm²) to the organic light emitting devices manufactured in Examples 1-1 to 1-46 and Comparative Examples 1-1 to 1-9, and the results are shown in Table 1 below. Lifetime T95 means the time required for the luminance to be reduced to 95% of the initial luminance (1000 nit).

TABLE 1

| | Electron | Voltage | Efficiency | Lifetime | Luminescent |
|---|---|---|---|---|---|
| | blocking layer | (V) | (cd/A) | T95(hr) | color |
| Example 1-1 | Compound 1 | 3.82 | 8.09 | 177 | Blue |
| Example 1-2 | Compound 2 | 3.77 | 7.92 | 178 | Blue |
| Example 1-3 | Compound 3 | 3.81 | 7.95 | 179 | Blue |
| Example 1-4 | Compound 4 | 3.75 | 7.92 | 179 | Blue |
| Example 1-5 | Compound 5 | 3.72 | 7.90 | 194 | Blue |
| Example 1-6 | Compound 6 | 3.74 | 8.56 | 253 | Blue |
| Example 1-7 | Compound 7 | 3.62 | 8.52 | 247 | Blue |
| Example 1-8 | Compound 8 | 3.73 | 8.69 | 237 | Blue |
| Example 1-9 | Compound 9 | 3.72 | 8.53 | 246 | Blue |
| Example 1-10 | Compound 10 | 3.69 | 8.50 | 228 | Blue |
| Example 1-11 | Compound 11 | 3.62 | 8.55 | 251 | Blue |
| Example 1-12 | Compound 12 | 3.68 | 8.73 | 234 | Blue |
| Example 1-13 | Compound 13 | 3.72 | 8.65 | 228 | Blue |
| Example 1-14 | Compound 14 | 3.64 | 8.65 | 228 | Blue |
| Example 1-15 | Compound 15 | 3.59 | 8.39 | 207 | Blue |
| Example 1-16 | Compound 16 | 3.63 | 8.38 | 216 | Blue |
| Example 1-17 | Compound 17 | 3.63 | 8.40 | 215 | Blue |
| Example 1-18 | Compound 18 | 3.56 | 8.32 | 227 | Blue |
| Example 1-19 | Compound 19 | 3.61 | 8.42 | 219 | Blue |
| Example 1-20 | Compound 20 | 3.64 | 8.53 | 213 | Blue |
| Example 1-21 | Compound 21 | 3.62 | 8.51 | 207 | Blue |
| Example 1-22 | Compound 22 | 3.61 | 8.34 | 216 | Blue |
| Example 1-23 | Compound 23 | 3.61 | 8.38 | 233 | Blue |
| Example 1-24 | Compound 24 | 3.63 | 8.46 | 220 | Blue |
| Example 1-25 | Compound 25 | 3.90 | 8.19 | 201 | Blue |
| Example 1-26 | Compound 26 | 3.84 | 8.15 | 205 | Blue |
| Example 1-27 | Compound 27 | 3.91 | 8.13 | 194 | Blue |
| Example 1-28 | Compound 28 | 3.86 | 8.20 | 211 | Blue |
| Example 1-29 | Compound 29 | 3.83 | 8.22 | 198 | Blue |
| Example 1-30 | Compound 30 | 3.91 | 8.18 | 204 | Blue |
| Example 1-31 | Compound 31 | 3.91 | 8.17 | 201 | Blue |
| Example 1-32 | Compound 32 | 3.60 | 8.71 | 236 | Blue |
| Example 1-33 | Compound 33 | 3.65 | 8.51 | 245 | Blue |
| Example 1-34 | Compound 34 | 3.61 | 8.54 | 248 | Blue |
| Example 1-35 | Compound 35 | 3.58 | 8.50 | 247 | Blue |
| Example 1-36 | Compound 36 | 3.57 | 8.66 | 245 | Blue |
| Example 1-37 | Compound 37 | 3.55 | 8.61 | 237 | Blue |
| Example 1-38 | Compound 38 | 3.62 | 8.65 | 228 | Blue |
| Example 1-39 | Compound 39 | 3.56 | 8.64 | 229 | Blue |
| Example 1-40 | Compound 40 | 3.90 | 8.13 | 202 | Blue |
| Example 1-41 | Compound 41 | 3.90 | 8.16 | 198 | Blue |
| Example 1-42 | Compound 42 | 3.88 | 8.18 | 193 | Blue |
| Example 1-43 | Compound 43 | 3.88 | 8.10 | 197 | Blue |
| Example 1-44 | Compound 44 | 3.93 | 8.12 | 210 | Blue |
| Example 1-45 | Compound 45 | 3.93 | 8.15 | 209 | Blue |
| Example 1-46 | Compound 46 | 3.89 | 8.34 | 189 | Blue |
| Comparative | Compound EB1 | 4.08 | 7.26 | 127 | Blue |
| Example 1-1 | | | | | |
| Comparative | Compound EB2 | 4.12 | 7.32 | 138 | Blue |
| Example 1-2 | | | | | |
| Comparative | Compound EB3 | 4.18 | 7.04 | 142 | Blue |
| Example 1-3 | | | | | |
| Comparative | Compound EB4 | 4.35 | 6.83 | 98 | Blue |
| Example 1-4 | | | | | |
| Comparative | Compound EB5 | 4.22 | 7.18 | 115 | Blue |
| Example 1-5 | | | | | |
| Comparative | Compound EB6 | 4.09 | 7.01 | 131 | Blue |
| Example 1-6 | | | | | |
| Comparative | Compound EB7 | 4.02 | 7.36 | 156 | Blue |
| Example 1-7 | | | | | |
| Comparative | Compound EB8 | 4.34 | 4.95 | 27 | Blue |
| Example 1-8 | | | | | |
| Comparative | Compound EB9 | 4.35 | 5.24 | 64 | Blue |
| Example 1-9 | | | | | |

As shown in Table 1, it was confirmed that the compounds of the present disclosure have excellent electron blocking capability, and thus, the organic light emitting device comprising the same as an electron blocking layer exhibits remarkable effects in terms of driving voltage, efficiency and lifetime.

[Description of Symbols]

| | |
|---|---|
| 1: substrate | 2: anode |
| 3: electron blocking layer | 4: light emitting layer |
| 5: cathode | 6: hole injection layer |
| 7: hole transport layer | 8: hole blocking layer |
| 9: electron transport and injection layer | |

The invention claimed is:

1. A compound of Chemical Formula 1: 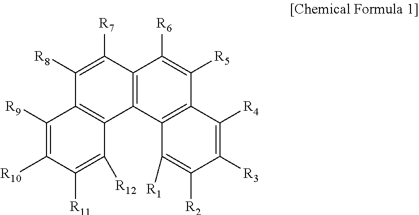
wherein in Chemical Formula 1:
any one of R₁ to R₁₂ is a substituent of Chemical Formula 2, and the rest are hydrogen or deuterium; 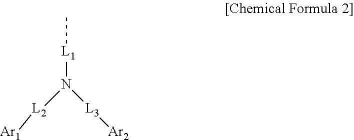
wherein in Chemical Formula 2:
L₁ is a substituted or unsubstituted phenylene, a substituted or unsubstituted biphenyldiyl, or a substituted or unsubstituted naphthalenediyl;
L₂ and L₃ are each independently a single, a substituted or unsubstituted C₆₋₆₀ arylene, or a substituted or unsubstituted C₂₋₆₀ heteroarylene containing at least one heteroatom selected from the group consisting of N, O and S; and
Ar₁ and Ar₂ are each independently a substituted or unsubstituted C₆₋₆₀ aryl or a substituted or unsubstituted C₂₋₆₀ heteroaryl containing at least one heteroatom selected from the group consisting of N, O and S.

2. The compound of claim 1, wherein:
L₁ is any one selected from the group consisting of:

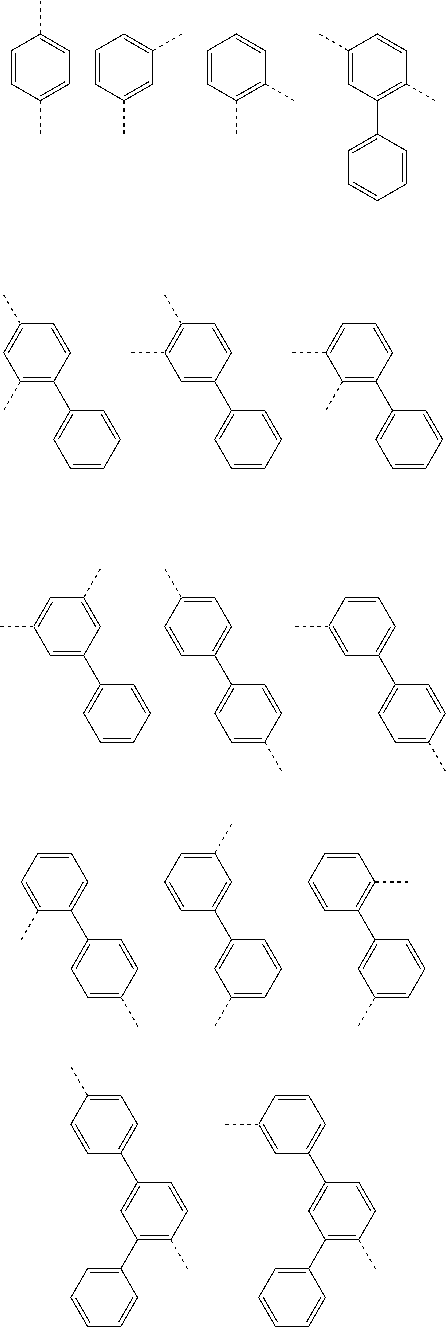

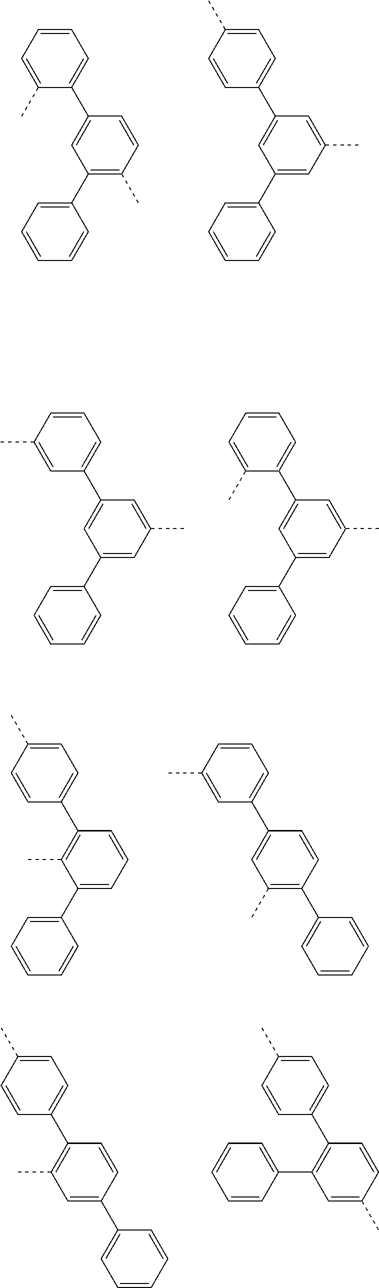

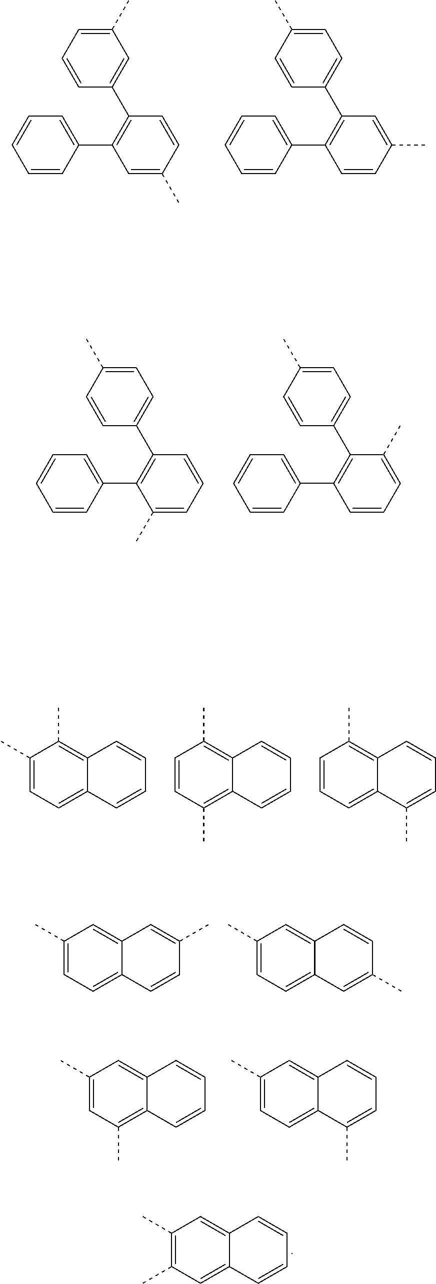

3. The compound of claim 1, wherein:
L₂ and L₃ are each independently a single bond, phenylene, phenylene substituted with one phenyl, biphenyldiyl, or naphthalenediyl.

4. The compound of claim 1, wherein:
L₂ and L₃ are each independently a single bond or any one selected from the group consisting of:

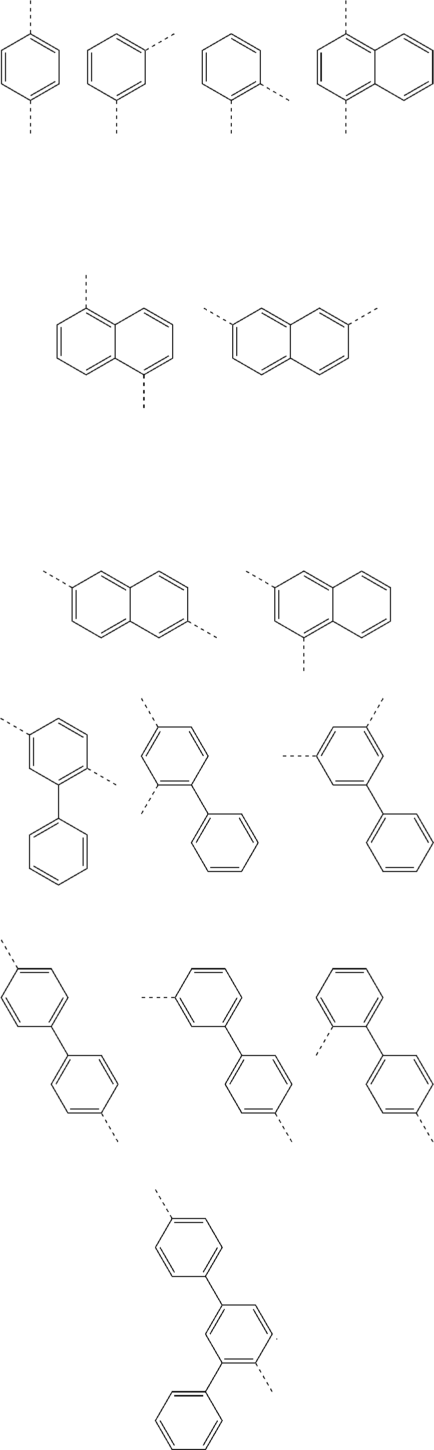

5. The compound of claim 1, wherein:
Ar₁ and Ar₂ are each independently phenyl, biphenylyl, terphenylyl, naphthyl, phenanthrenyl, phenyl naphthyl, dibenzofuranyl, dibenzothiophenyl, phenyl carbazolyl, dimethyl fluorenyl, benzonaphthofuranyl, or benzonaphthothiophenyl.

6. The compound of claim 1, wherein:
Ar₁ and Ar₂ are each independently any one selected from the group consisting of:

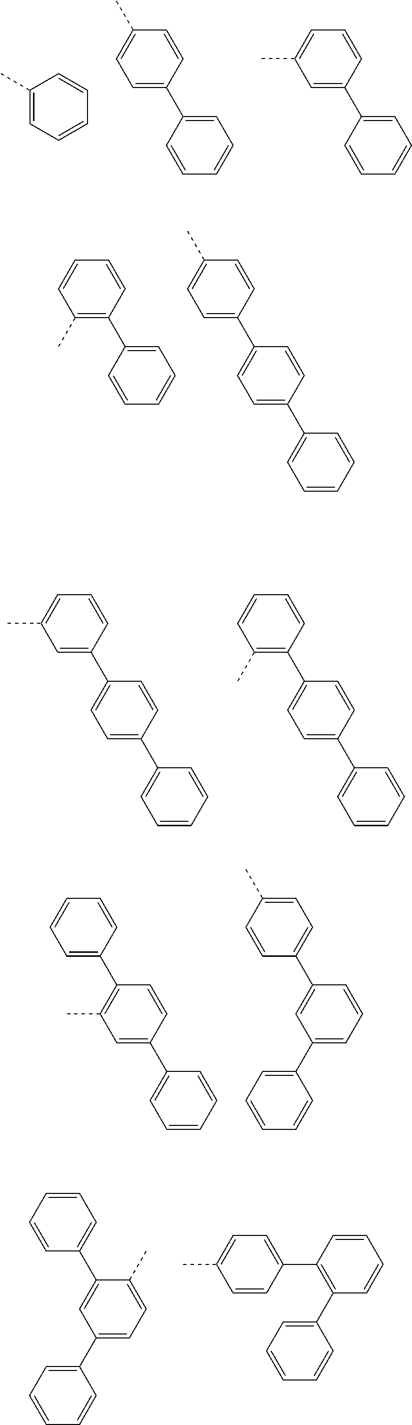

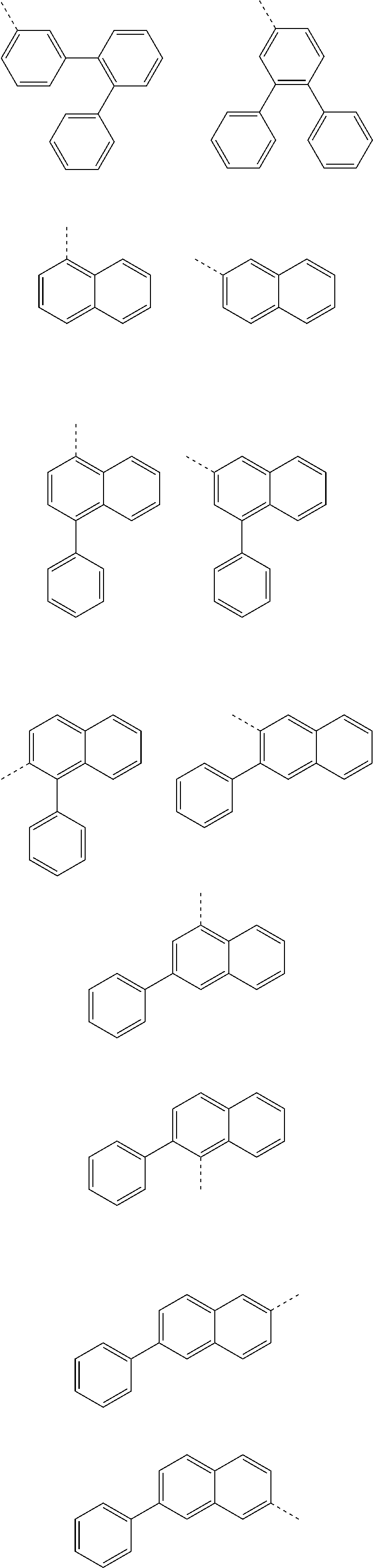

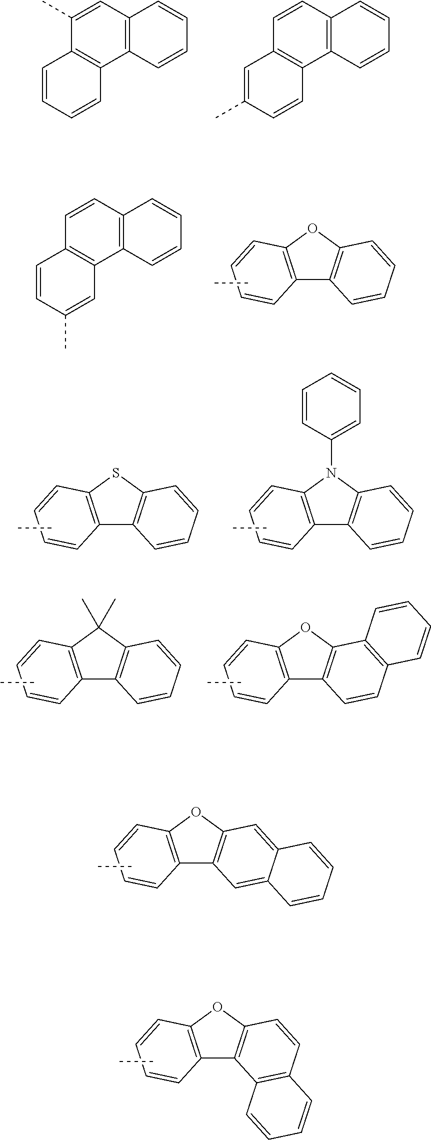

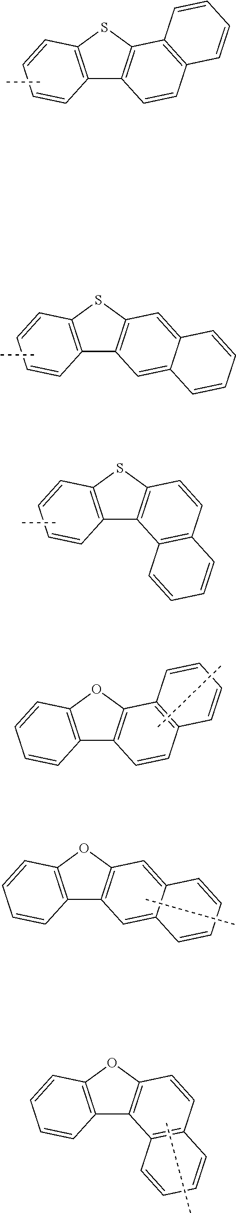

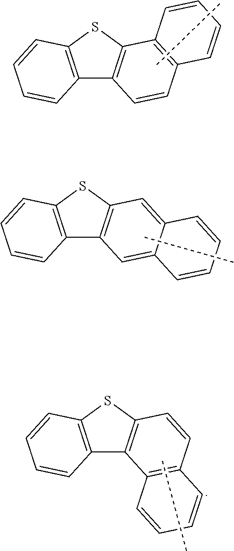

7. The compound of claim 1, wherein
any one of Ar₁ and Ar₂ is a substituted or unsubstituted C₆₋₆₀ aryl.

8. The compound of claim 1, wherein:
the compound of Chemical Formula 1 is any one compound selected from the group consisting of.

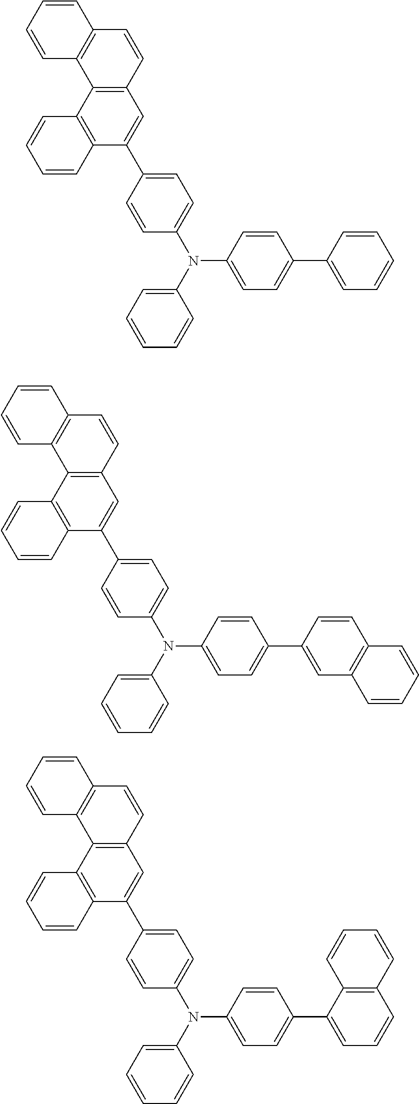 [SMILES: c1ccc(-c2ccc(N(c3ccccc3)c3ccc(-c4cc5ccc6ccccc6c5c5ccccc45)cc3)cc2)cc1 ; c1ccc(N(c2ccc(-c3ccc4ccccc4c3)cc2)c2ccc(-c3cc4ccc5ccccc5c4c4ccccc34)cc2)cc1 ; c1ccc(N(c2ccc(-c3cccc4ccccc34)cc2)c2ccc(-c3cc4ccc5ccccc5c4c4ccccc34)cc2)cc1]

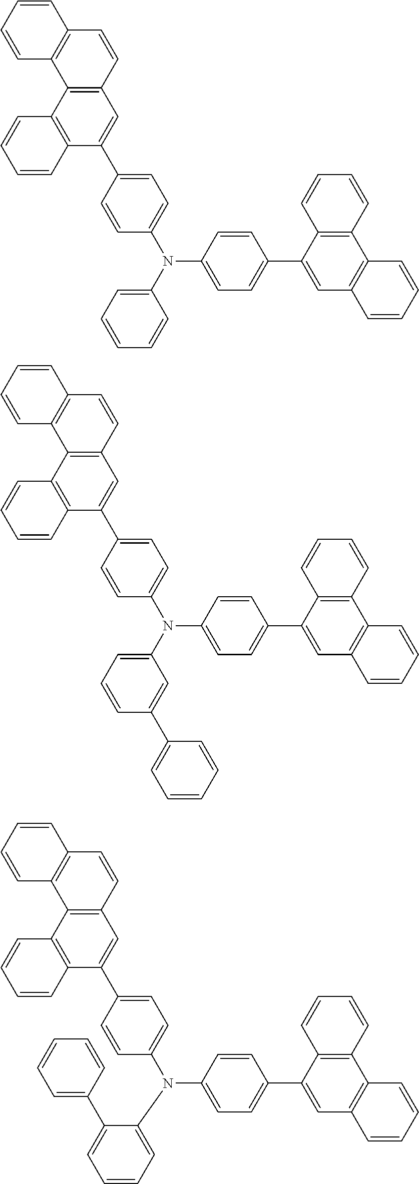 [SMILES: c1ccc(N(c2ccc(-c3cc4ccccc4c4ccccc34)cc2)c2ccc(-c3cc4ccc5ccccc5c4c4ccccc34)cc2)cc1 ; c1ccc(-c2cccc(N(c3ccc(-c4cc5ccccc5c5ccccc45)cc3)c3ccc(-c4cc5ccc6ccccc6c5c5ccccc45)cc3)c2)cc1 ; c1ccc(-c2ccccc2N(c2ccc(-c3cc4ccccc4c4ccccc34)cc2)c2ccc(-c3cc4ccc5ccccc5c4c4ccccc34)cc2)cc1]

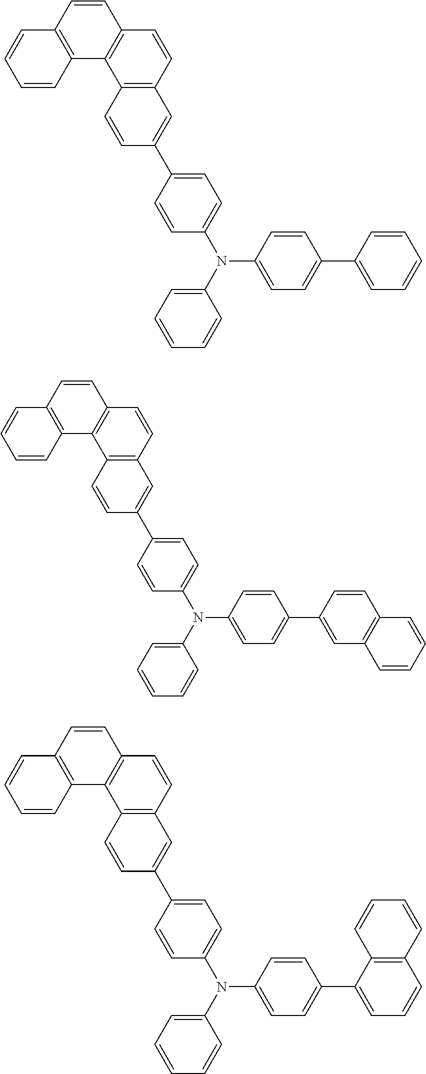 [SMILES: c1ccc(-c2ccc(N(c3ccccc3)c3ccc(-c4ccc5c(ccc6ccc7ccccc7c65)c4)cc3)cc2)cc1 ; c1ccc(N(c2ccc(-c3ccc4ccccc4c3)cc2)c2ccc(-c3ccc4c(ccc5ccc6ccccc6c54)c3)cc2)cc1 ; c1ccc(N(c2ccc(-c3ccc4c(ccc5ccc6ccccc6c54)c3)cc2)c2ccc(-c3cccc4ccccc34)cc2)cc1]

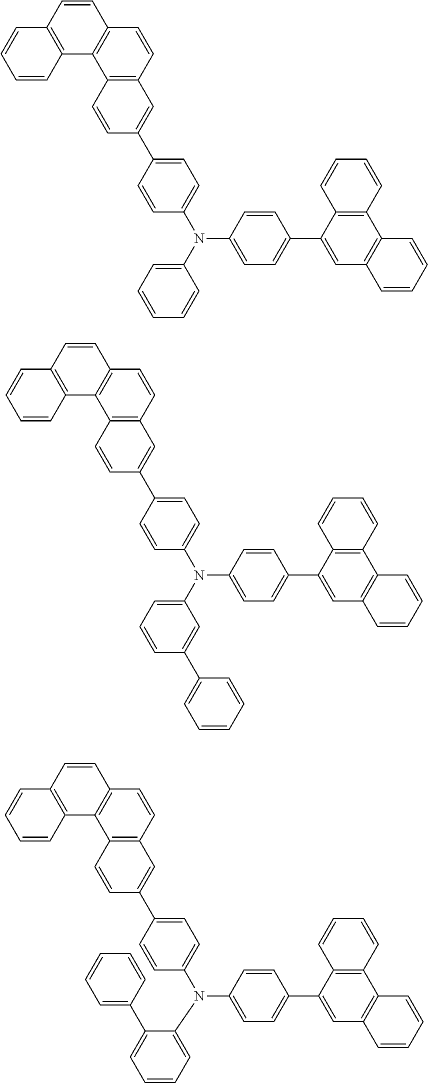 [SMILES: c1ccc(N(c2ccc(-c3ccc4c(ccc5ccc6ccccc6c54)c3)cc2)c2ccc(-c3cc4ccccc4c4ccccc34)cc2)cc1 ; c1ccc(-c2cccc(N(c3ccc(-c4ccc5c(ccc6ccc7ccccc7c65)c4)cc3)c3ccc(-c4cc5ccccc5c5ccccc45)cc3)c2)cc1 ; c1ccc(-c2ccccc2N(c2ccc(-c3ccc4c(ccc5ccc6ccccc6c54)c3)cc2)c2ccc(-c3cc4ccccc4c4ccccc34)cc2)cc1]

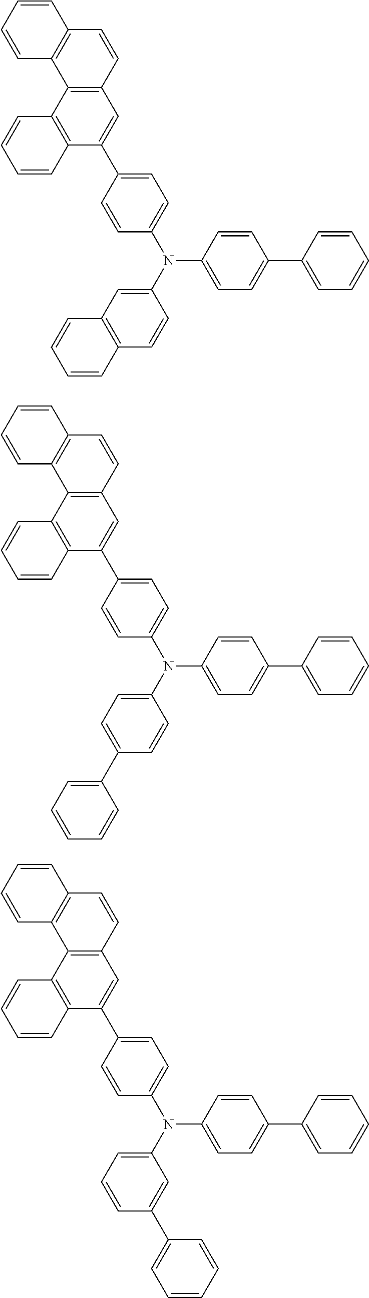

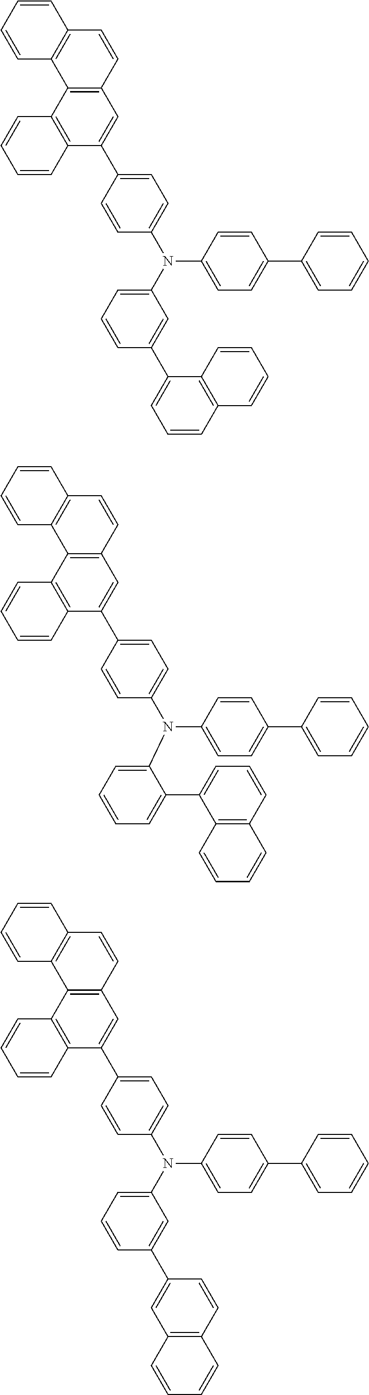 [SMILES: c1ccc(-c2ccc(N(c3ccc(-c4cc5ccc6ccccc6c5c5ccccc45)cc3)c3cccc(-c4cccc5ccccc45)c3)cc2)cc1 ; c1ccc(-c2ccc(N(c3ccc(-c4cc5ccc6ccccc6c5c5ccccc45)cc3)c3ccccc3-c3cccc4ccccc34)cc2)cc1 ; c1ccc(-c2ccc(N(c3ccc(-c4cc5ccc6ccccc6c5c5ccccc45)cc3)c3cccc(-c4ccc5ccccc5c4)c3)cc2)cc1]

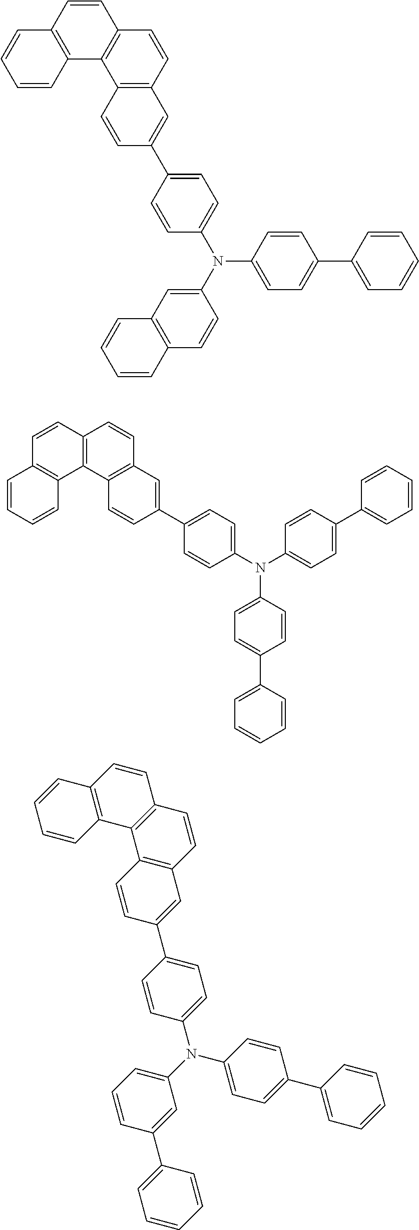

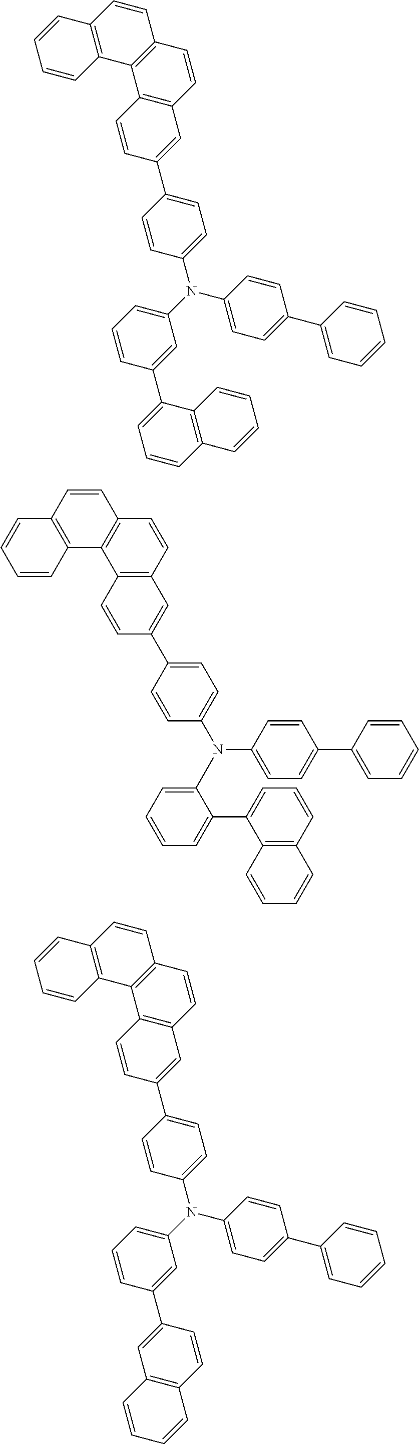 [SMILES: c1ccc(-c2ccc(N(c3ccc(-c4ccc5c(ccc6ccc7ccccc7c65)c4)cc3)c3cccc(-c4cccc5ccccc45)c3)cc2)cc1 ; c1ccc(-c2ccc(N(c3ccc(-c4ccc5c(ccc6ccc7ccccc7c65)c4)cc3)c3ccccc3-c3cccc4ccccc34)cc2)cc1 ; c1ccc(-c2ccc(N(c3ccc(-c4ccc5c(ccc6ccc7ccccc7c65)c4)cc3)c3cccc(-c4ccc5ccccc5c4)c3)cc2)cc1]

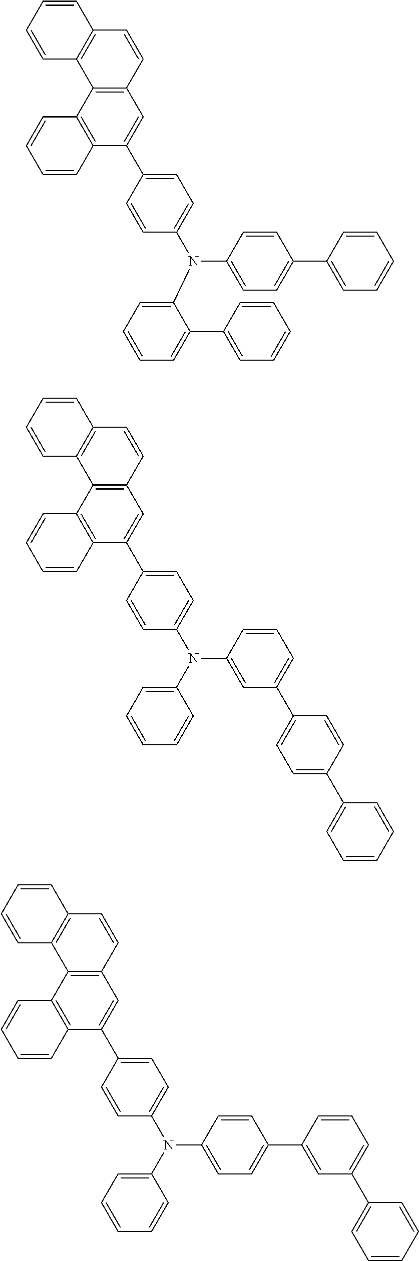 [SMILES: c1ccc(-c2ccc(N(c3ccc(-c4cc5ccc6ccccc6c5c5ccccc45)cc3)c3ccccc3-c3ccccc3)cc2)cc1 ; c1ccc(-c2ccc(-c3cccc(N(c4ccccc4)c4ccc(-c5cc6ccc7ccccc7c6c6ccccc56)cc4)c3)cc2)cc1 ; c1ccc(-c2cccc(-c3ccc(N(c4ccccc4)c4ccc(-c5cc6ccc7ccccc7c6c6ccccc56)cc4)cc3)c2)cc1]

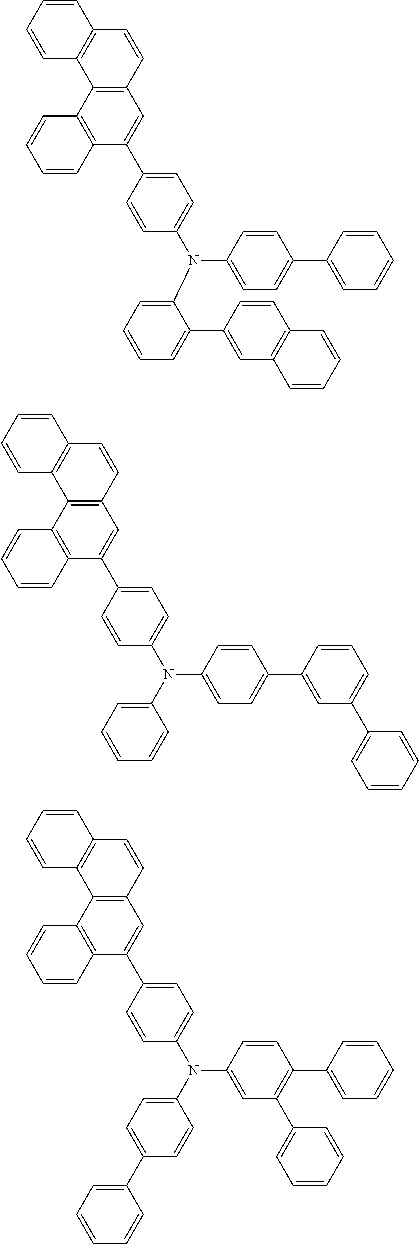 [SMILES: c1ccc(-c2ccc(N(c3ccc(-c4cc5ccc6ccccc6c5c5ccccc45)cc3)c3ccccc3-c3ccc4ccccc4c3)cc2)cc1 ; c1ccc(-c2cccc(-c3ccc(N(c4ccccc4)c4ccc(-c5cc6ccc7ccccc7c6c6ccccc56)cc4)cc3)c2)cc1 ; c1ccc(-c2ccc(N(c3ccc(-c4cc5ccc6ccccc6c5c5ccccc45)cc3)c3ccc(-c4ccccc4)c(-c4ccccc4)c3)cc2)cc1]

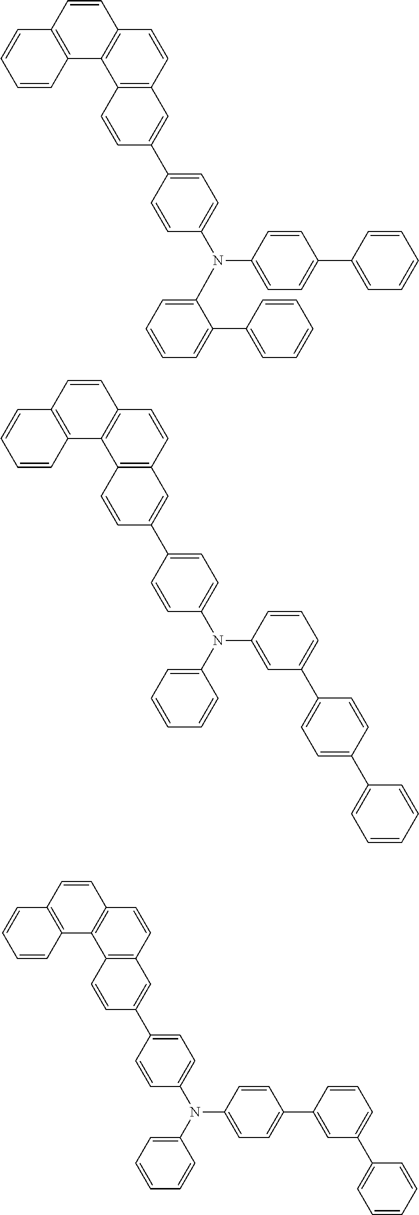 [SMILES: c1ccc(-c2ccc(N(c3ccc(-c4ccc5c(ccc6ccc7ccccc7c65)c4)cc3)c3ccccc3-c3ccccc3)cc2)cc1 ; c1ccc(-c2ccc(-c3cccc(N(c4ccccc4)c4ccc(-c5ccc6c(ccc7ccc8ccccc8c76)c5)cc4)c3)cc2)cc1 ; c1ccc(-c2cccc(-c3ccc(N(c4ccccc4)c4ccc(-c5ccc6c(ccc7ccc8ccccc8c76)c5)cc4)cc3)c2)cc1]

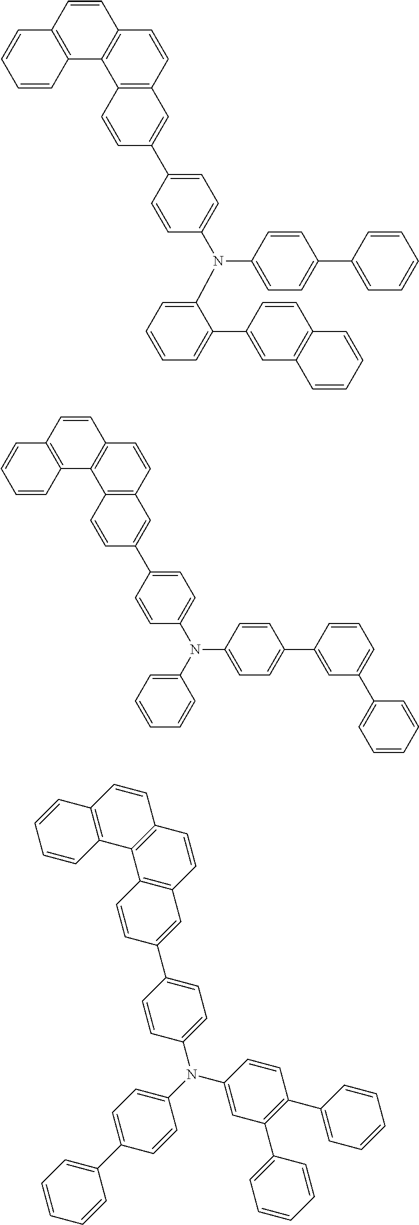 [SMILES: c1ccc(-c2ccc(N(c3ccc(-c4ccc5c(ccc6ccc7ccccc7c65)c4)cc3)c3ccccc3-c3ccc4ccccc4c3)cc2)cc1 ; c1ccc(-c2cccc(-c3ccc(N(c4ccccc4)c4ccc(-c5ccc6c(ccc7ccc8ccccc8c76)c5)cc4)cc3)c2)cc1 ; c1ccc(-c2ccc(N(c3ccc(-c4ccc5c(ccc6ccc7ccccc7c65)c4)cc3)c3ccc(-c4ccccc4)c(-c4ccccc4)c3)cc2)cc1]

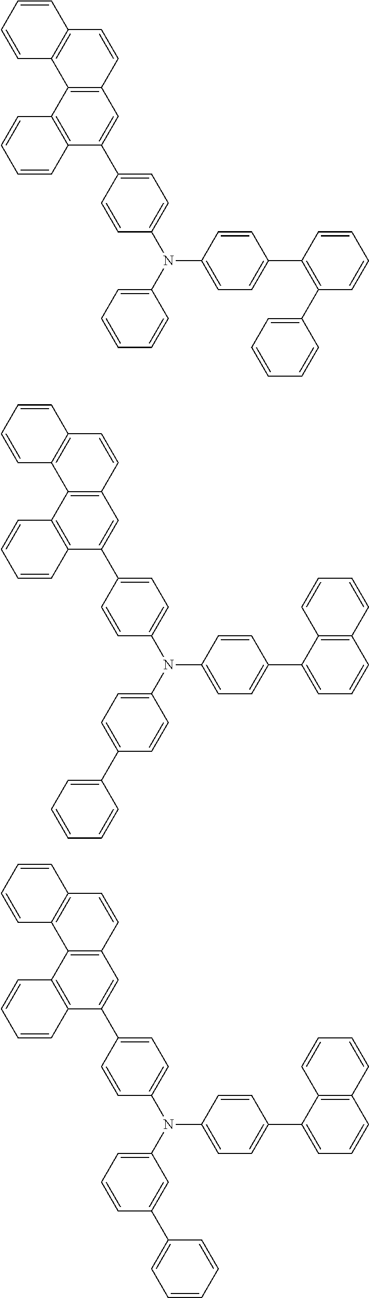

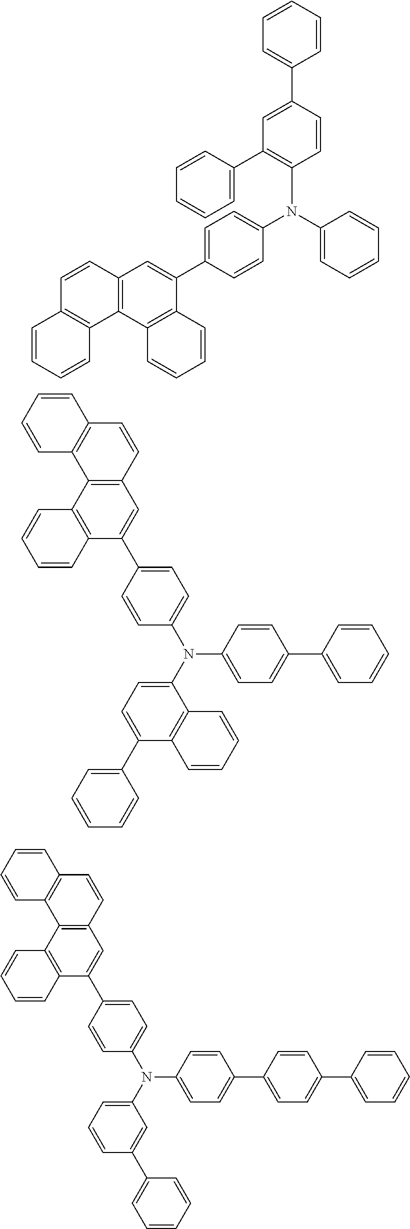

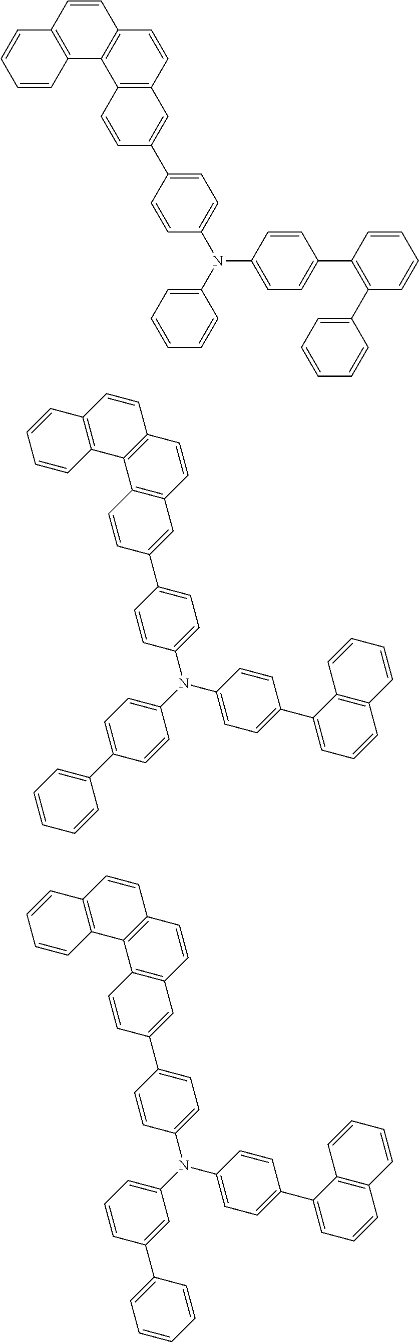 [SMILES: c1ccc(-c2ccccc2-c2ccc(N(c3ccccc3)c3ccc(-c4ccc5c(ccc6ccc7ccccc7c65)c4)cc3)cc2)cc1 ; c1ccc(-c2ccc(N(c3ccc(-c4ccc5c(ccc6ccc7ccccc7c65)c4)cc3)c3ccc(-c4cccc5ccccc45)cc3)cc2)cc1 ; c1ccc(-c2cccc(N(c3ccc(-c4ccc5c(ccc6ccc7ccccc7c65)c4)cc3)c3ccc(-c4cccc5ccccc45)cc3)c2)cc1]

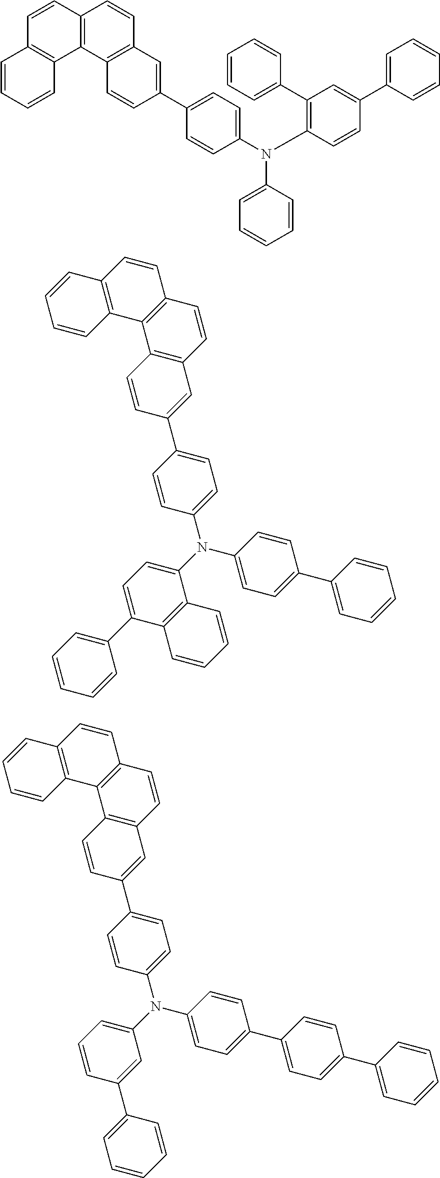

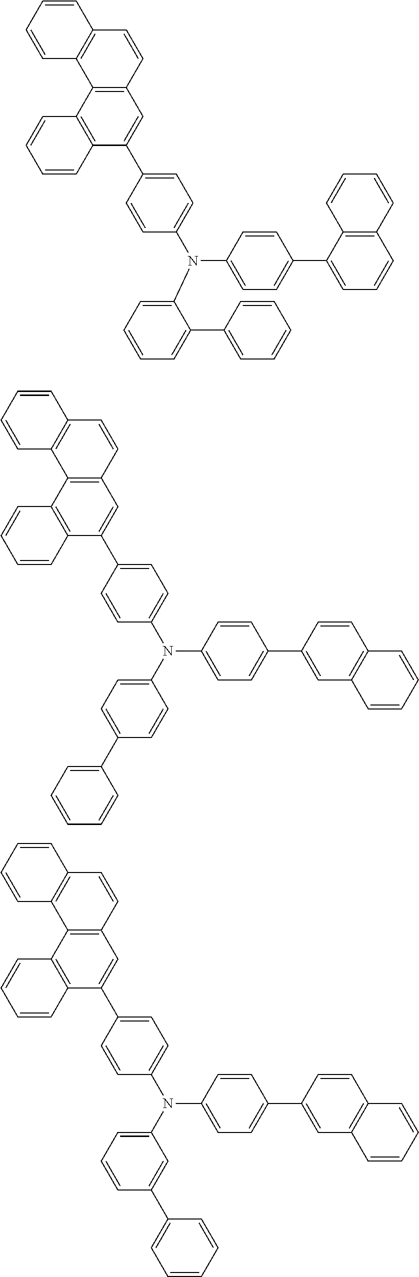

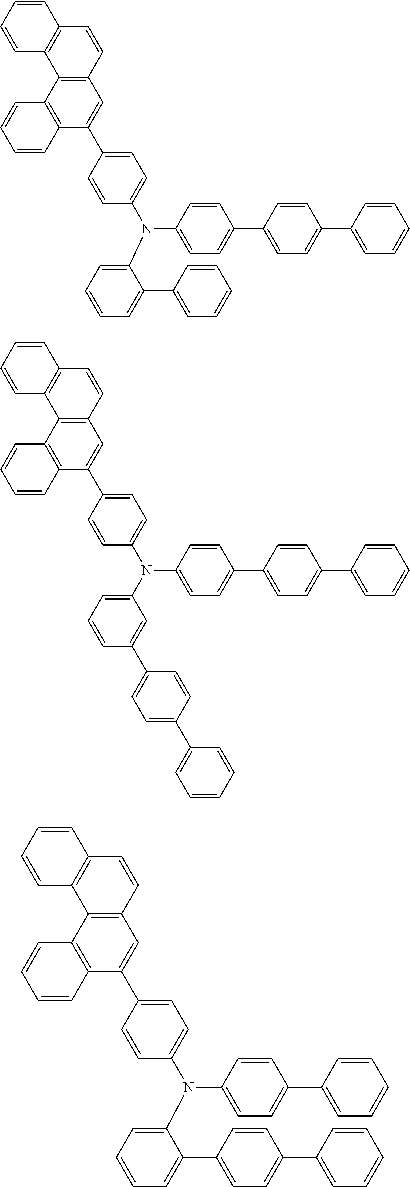 [SMILES: c1ccc(-c2ccc(-c3ccc(N(c4ccc(-c5cc6ccc7ccccc7c6c6ccccc56)cc4)c4ccccc4-c4ccccc4)cc3)cc2)cc1 ; c1ccc(-c2ccc(-c3ccc(N(c4ccc(-c5cc6ccc7ccccc7c6c6ccccc56)cc4)c4cccc(-c5ccc(-c6ccccc6)cc5)c4)cc3)cc2)cc1 ; c1ccc(-c2ccc(-c3ccccc3N(c3ccc(-c4ccccc4)cc3)c3ccc(-c4cc5ccc6ccccc6c5c5ccccc45)cc3)cc2)cc1]

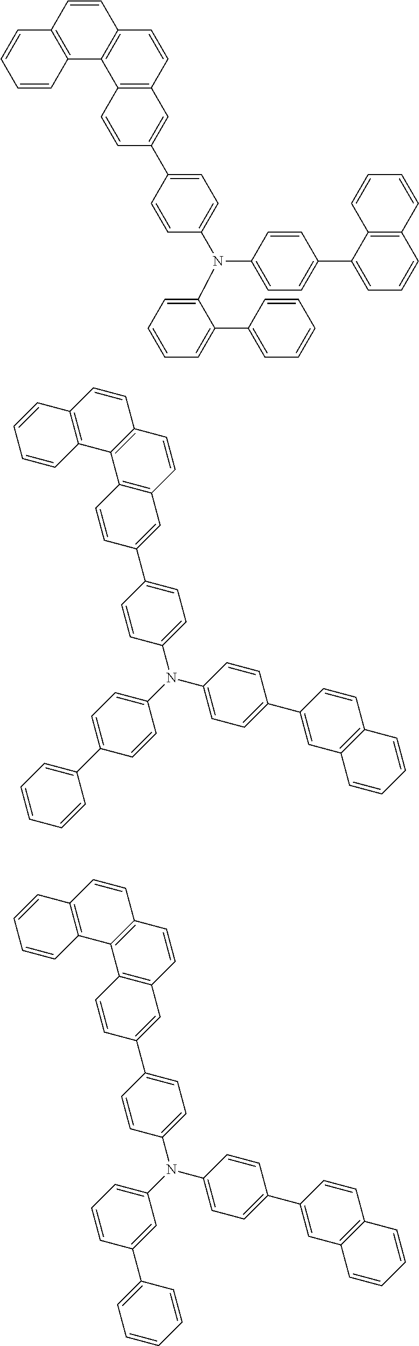 [SMILES: c1ccc(-c2ccccc2N(c2ccc(-c3ccc4c(ccc5ccc6ccccc6c54)c3)cc2)c2ccc(-c3cccc4ccccc34)cc2)cc1 ; c1ccc(-c2ccc(N(c3ccc(-c4ccc5ccccc5c4)cc3)c3ccc(-c4ccc5c(ccc6ccc7ccccc7c65)c4)cc3)cc2)cc1 ; c1ccc(-c2cccc(N(c3ccc(-c4ccc5ccccc5c4)cc3)c3ccc(-c4ccc5c(ccc6ccc7ccccc7c65)c4)cc3)c2)cc1]

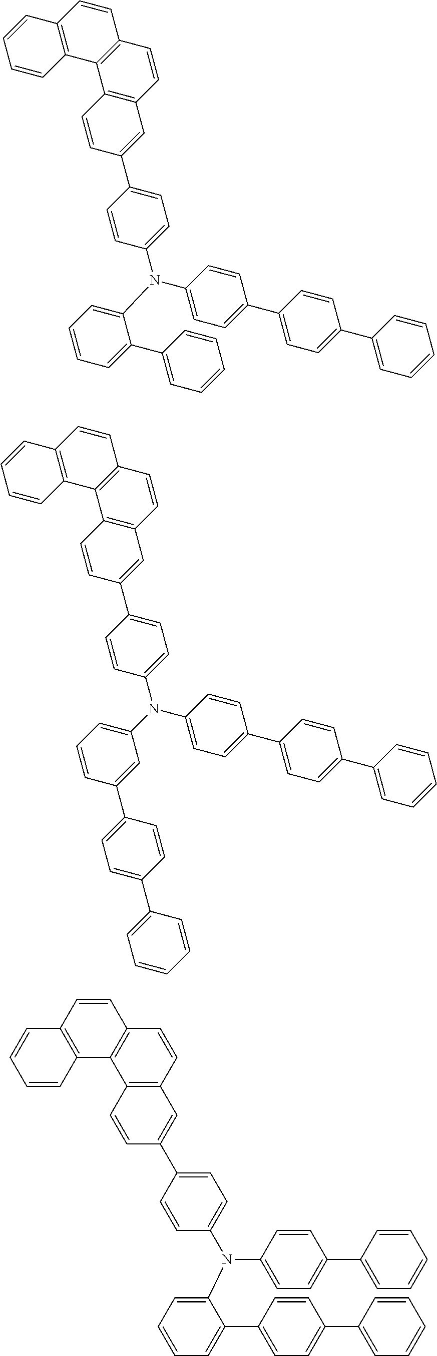

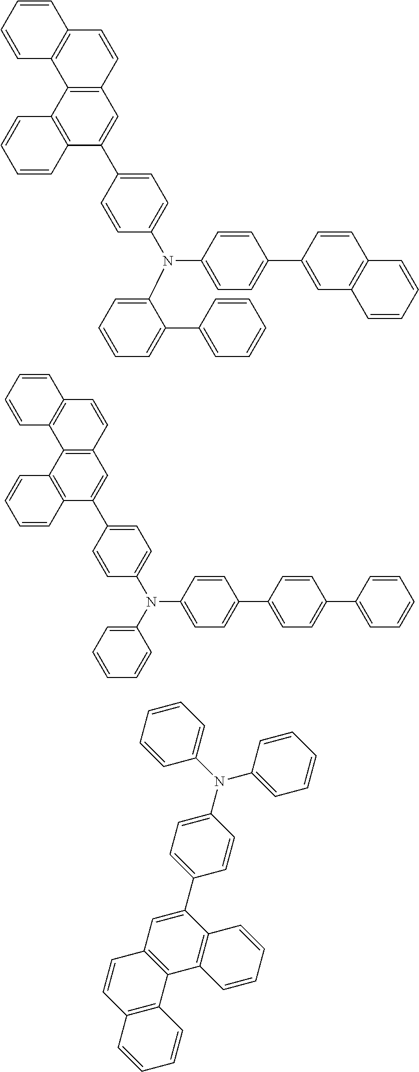

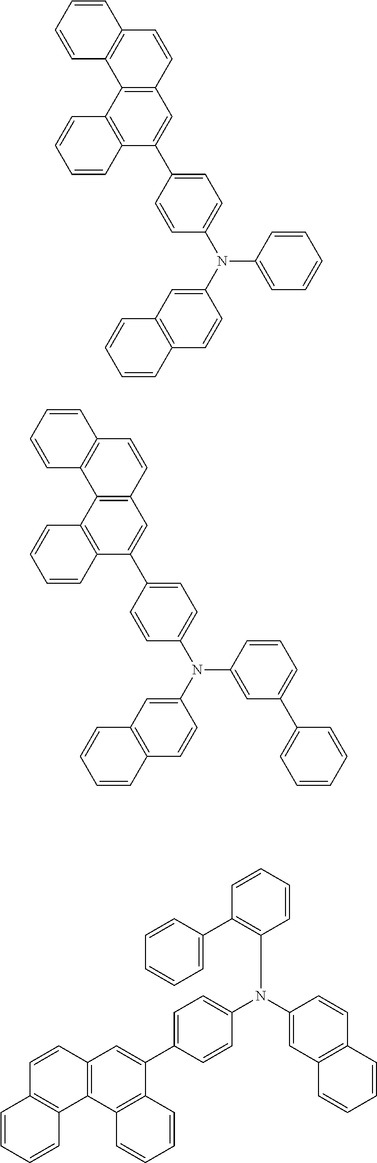 [SMILES: c1ccc(N(c2ccc(-c3cc4ccc5ccccc5c4c4ccccc34)cc2)c2ccc3ccccc3c2)cc1 ; c1ccc(-c2cccc(N(c3ccc(-c4cc5ccc6ccccc6c5c5ccccc45)cc3)c3ccc4ccccc4c3)c2)cc1 ; c1ccc(-c2ccccc2N(c2ccc(-c3cc4ccc5ccccc5c4c4ccccc34)cc2)c2ccc3ccccc3c2)cc1]

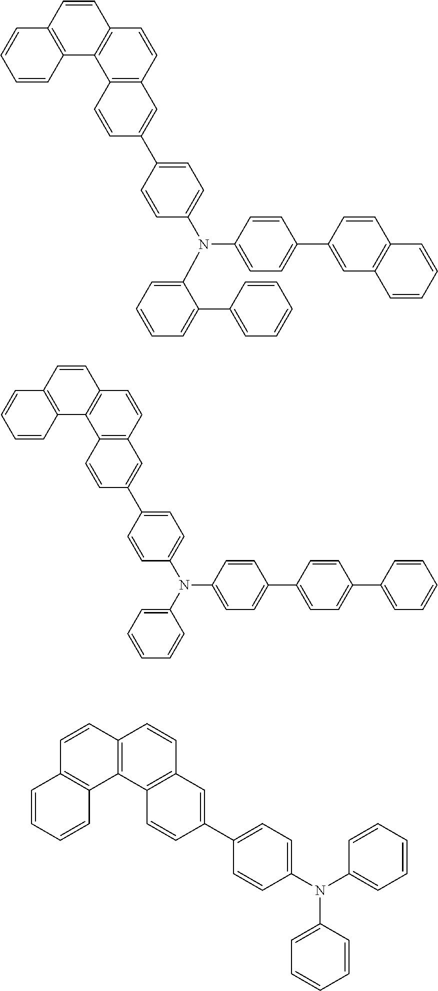 [SMILES: c1ccc(-c2ccccc2N(c2ccc(-c3ccc4ccccc4c3)cc2)c2ccc(-c3ccc4c(ccc5ccc6ccccc6c54)c3)cc2)cc1 ; c1ccc(-c2ccc(-c3ccc(N(c4ccccc4)c4ccc(-c5ccc6c(ccc7ccc8ccccc8c76)c5)cc4)cc3)cc2)cc1 ; c1ccc(N(c2ccccc2)c2ccc(-c3ccc4c(ccc5ccc6ccccc6c54)c3)cc2)cc1]

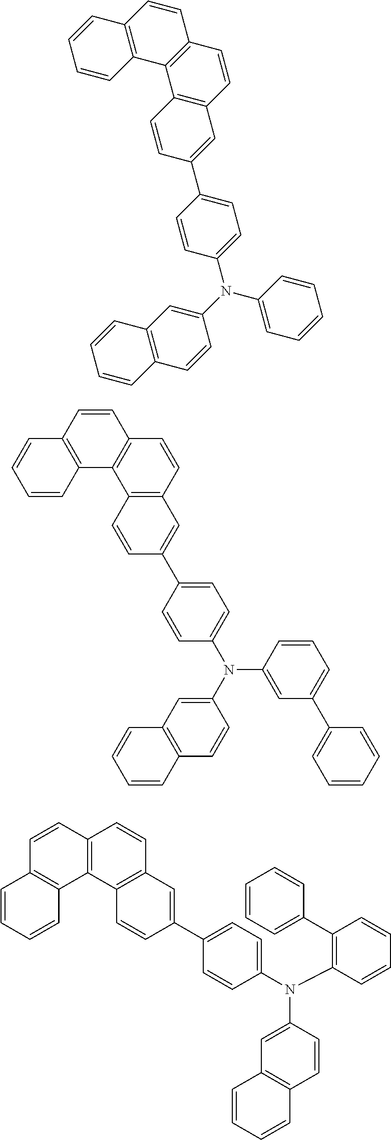

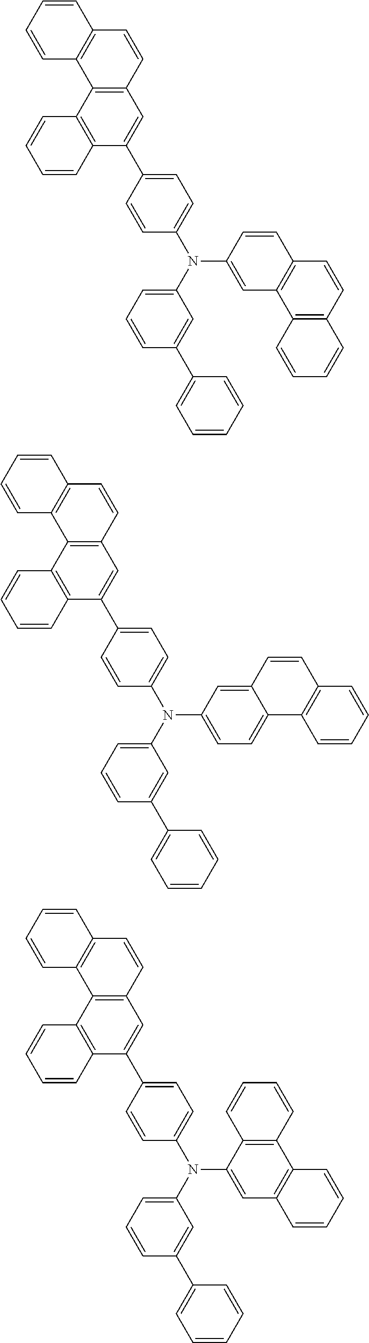 [SMILES: c1ccc(-c2cccc(N(c3ccc(-c4cc5ccc6ccccc6c5c5ccccc45)cc3)c3ccc4ccc5ccccc5c4c3)c2)cc1 ; c1ccc(-c2cccc(N(c3ccc(-c4cc5ccc6ccccc6c5c5ccccc45)cc3)c3ccc4c(ccc5ccccc54)c3)c2)cc1 ; c1ccc(-c2cccc(N(c3ccc(-c4cc5ccc6ccccc6c5c5ccccc45)cc3)c3cc4ccccc4c4ccccc34)c2)cc1]

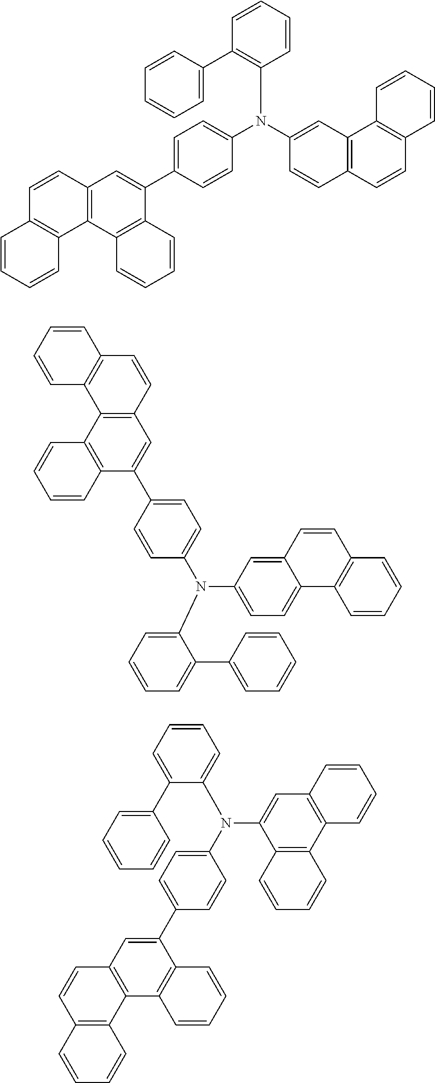

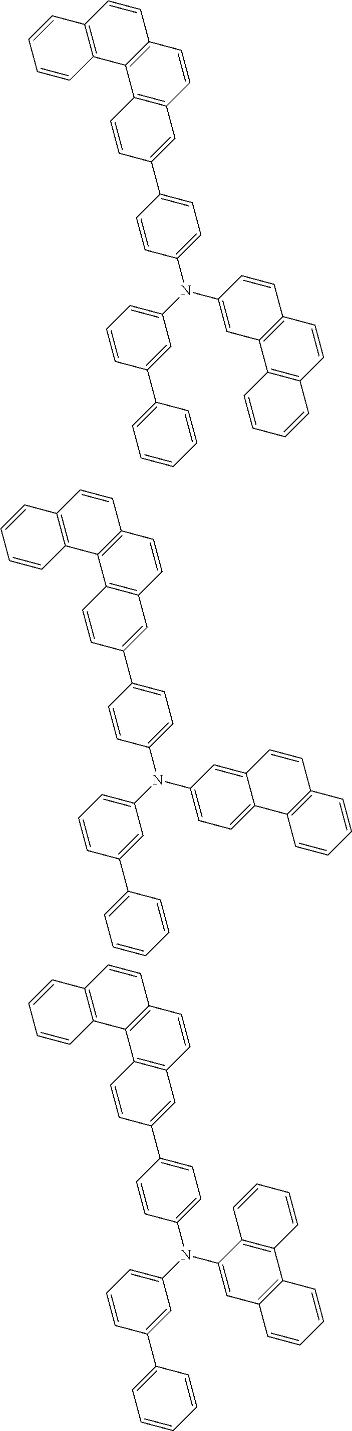

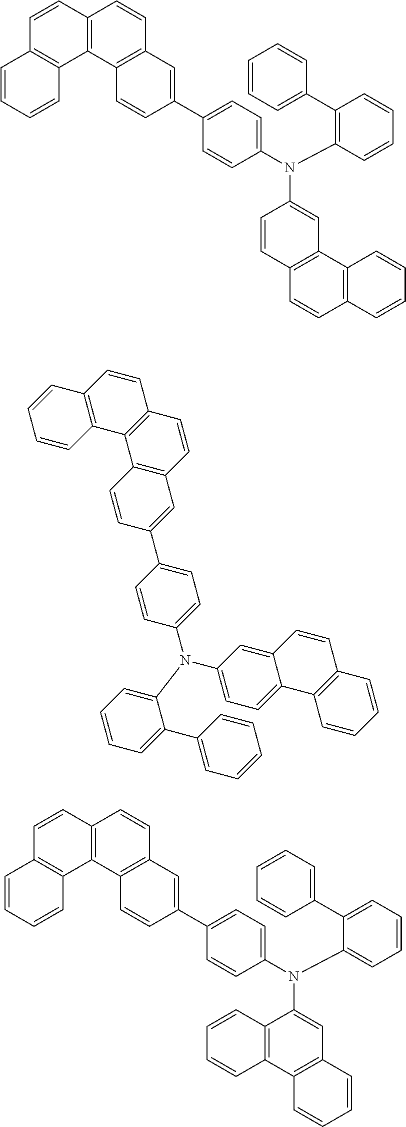 [SMILES: c1ccc(-c2ccccc2N(c2ccc(-c3ccc4c(ccc5ccc6ccccc6c54)c3)cc2)c2ccc3ccc4ccccc4c3c2)cc1 ; c1ccc(-c2ccccc2N(c2ccc(-c3ccc4c(ccc5ccc6ccccc6c54)c3)cc2)c2ccc3c(ccc4ccccc43)c2)cc1 ; c1ccc(-c2ccccc2N(c2ccc(-c3ccc4c(ccc5ccc6ccccc6c54)c3)cc2)c2cc3ccccc3c3ccccc23)cc1]

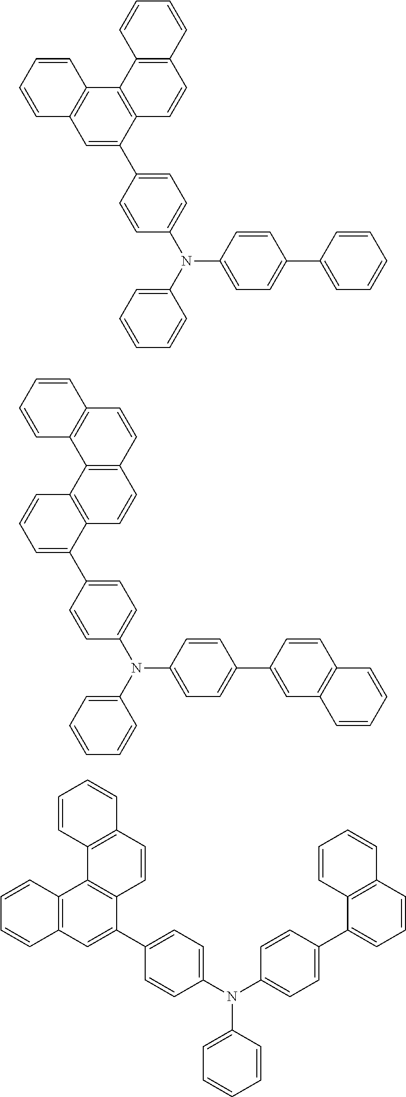

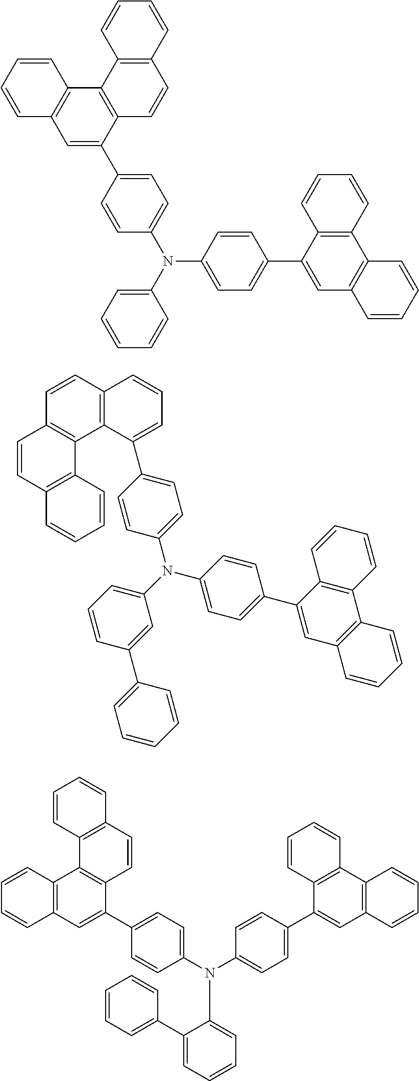

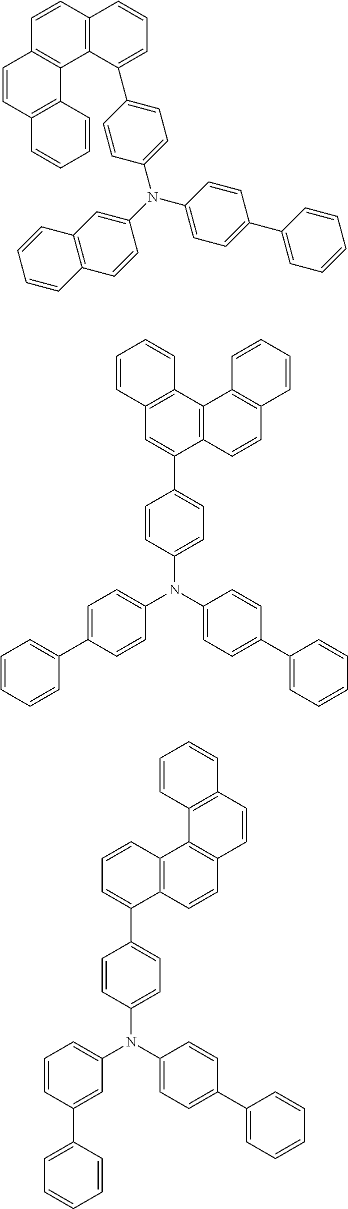 [SMILES: c1ccc(-c2ccc(N(c3ccc(-c4cccc5ccc6ccc7ccccc7c6c45)cc3)c3ccc4ccccc4c3)cc2)cc1 ; c1ccc(-c2ccc(N(c3ccc(-c4ccccc4)cc3)c3ccc(-c4cc5ccccc5c5c4ccc4ccccc45)cc3)cc2)cc1 ; c1ccc(-c2ccc(N(c3ccc(-c4cccc5c4ccc4ccc6ccccc6c45)cc3)c3cccc(-c4ccccc4)c3)cc2)cc1]

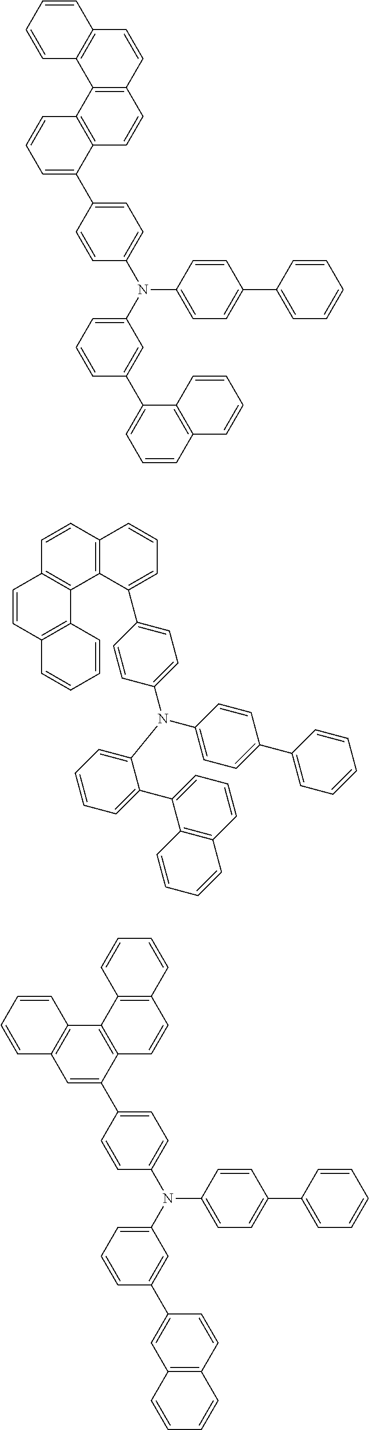 [SMILES: c1ccc(-c2ccc(N(c3ccc(-c4cccc5c4ccc4ccc6ccccc6c45)cc3)c3cccc(-c4cccc5ccccc45)c3)cc2)cc1 ; c1ccc(-c2ccc(N(c3ccc(-c4cccc5ccc6ccc7ccccc7c6c45)cc3)c3ccccc3-c3cccc4ccccc34)cc2)cc1 ; c1ccc(-c2ccc(N(c3ccc(-c4cc5ccccc5c5c4ccc4ccccc45)cc3)c3cccc(-c4ccc5ccccc5c4)c3)cc2)cc1]

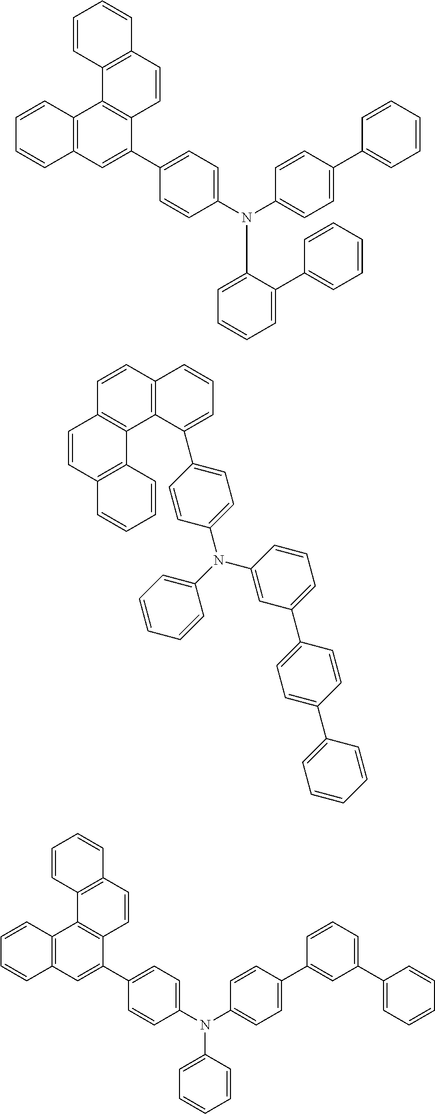 [SMILES: c1ccc(-c2ccc(N(c3ccc(-c4cc5ccccc5c5c4ccc4ccccc45)cc3)c3ccccc3-c3ccccc3)cc2)cc1 ; c1ccc(-c2ccc(-c3cccc(N(c4ccccc4)c4ccc(-c5cccc6ccc7ccc8ccccc8c7c56)cc4)c3)cc2)cc1 ; c1ccc(-c2cccc(-c3ccc(N(c4ccccc4)c4ccc(-c5cc6ccccc6c6c5ccc5ccccc56)cc4)cc3)c2)cc1]

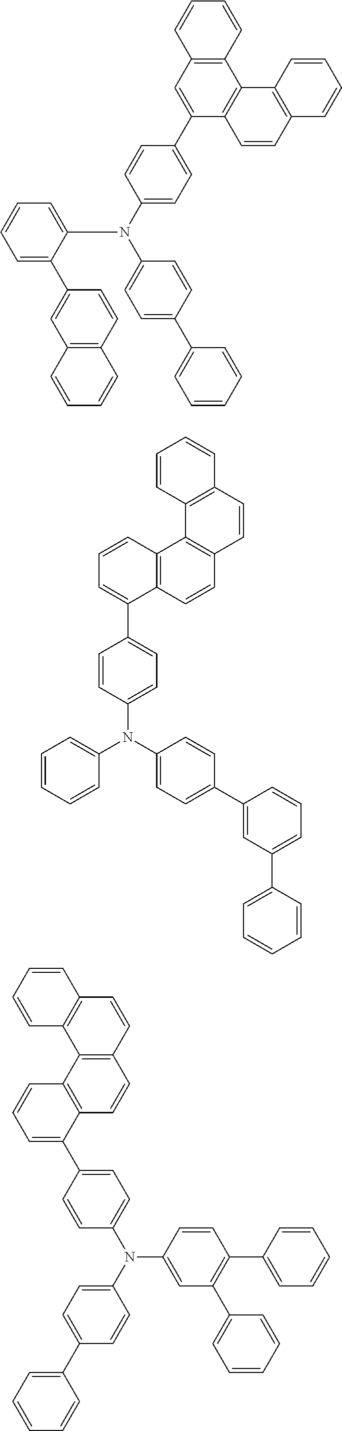

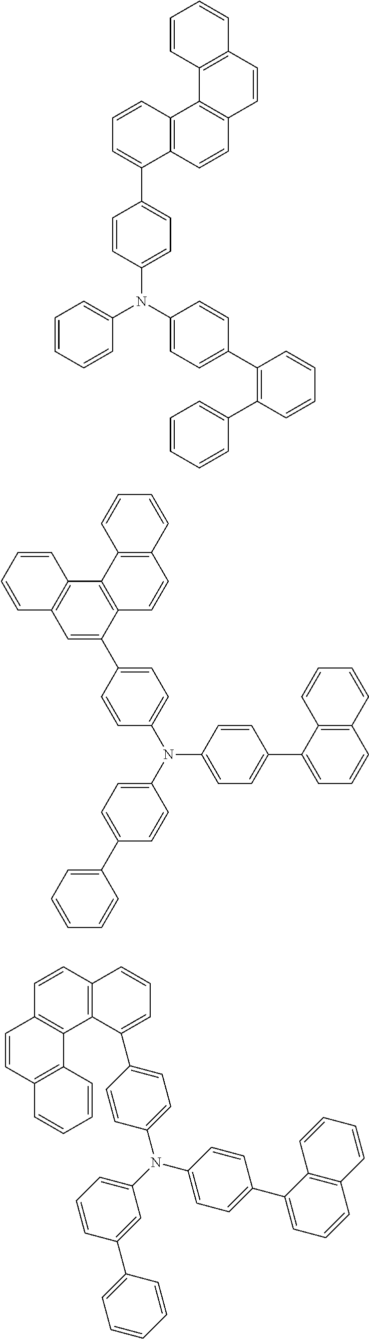

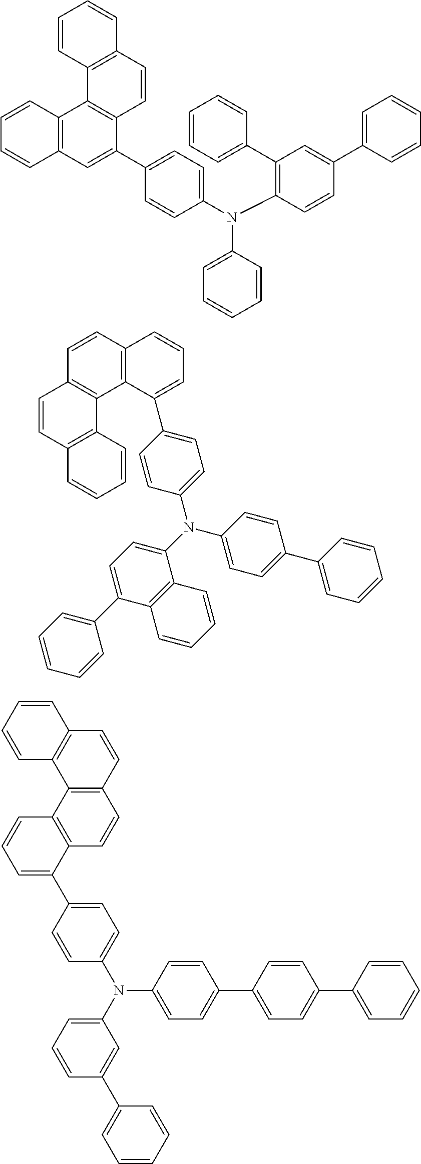

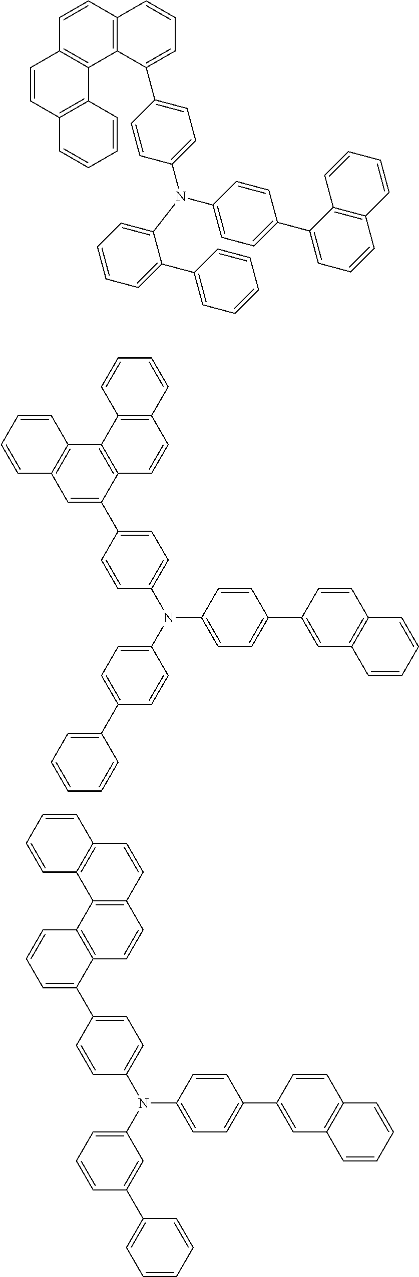

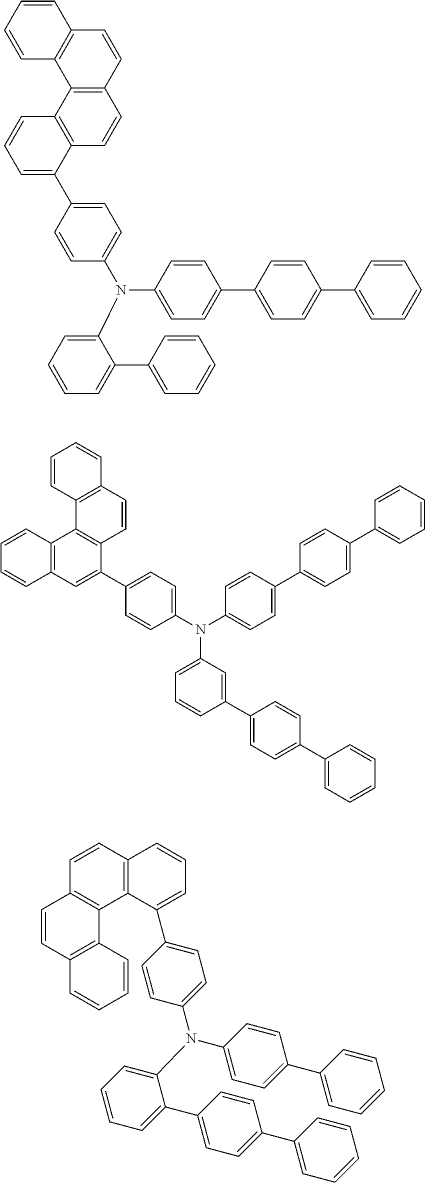 [SMILES: c1ccc(-c2ccc(-c3ccc(N(c4ccc(-c5cccc6c5ccc5ccc7ccccc7c56)cc4)c4ccccc4-c4ccccc4)cc3)cc2)cc1 ; c1ccc(-c2ccc(-c3ccc(N(c4ccc(-c5cc6ccccc6c6c5ccc5ccccc56)cc4)c4cccc(-c5ccc(-c6ccccc6)cc5)c4)cc3)cc2)cc1 ; c1ccc(-c2ccc(-c3ccccc3N(c3ccc(-c4ccccc4)cc3)c3ccc(-c4cccc5ccc6ccc7ccccc7c6c45)cc3)cc2)cc1]

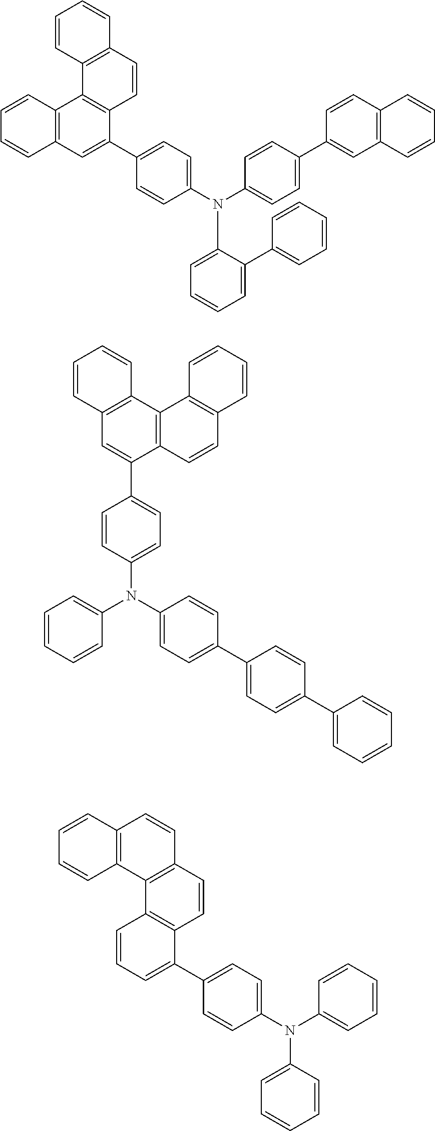

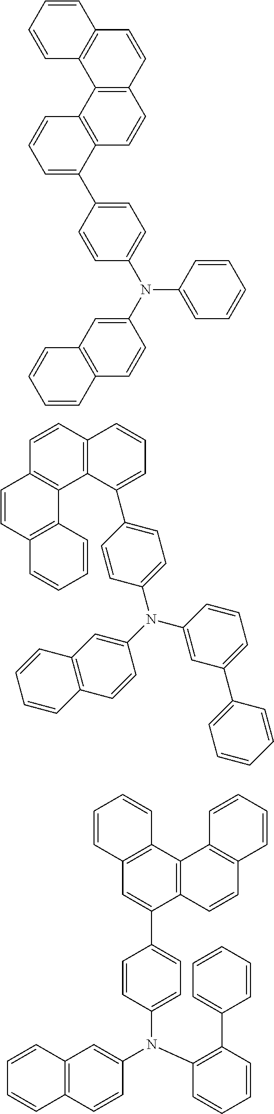

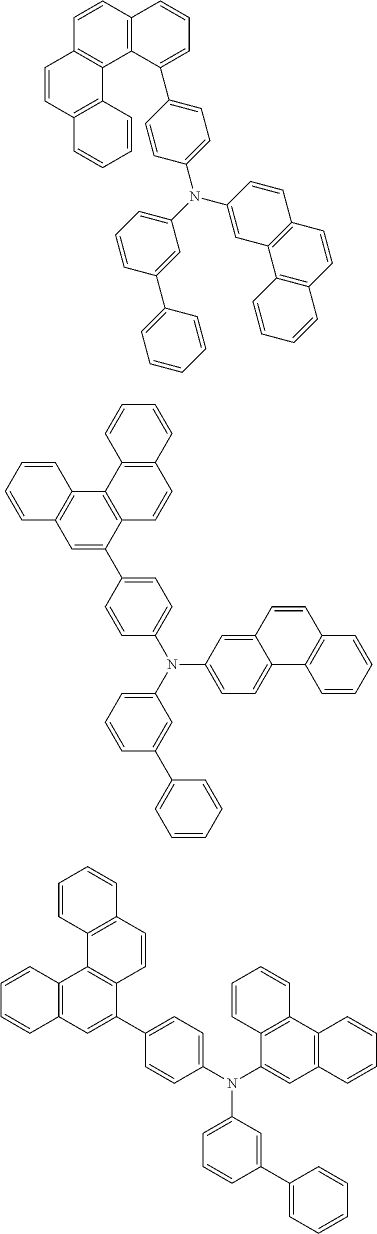

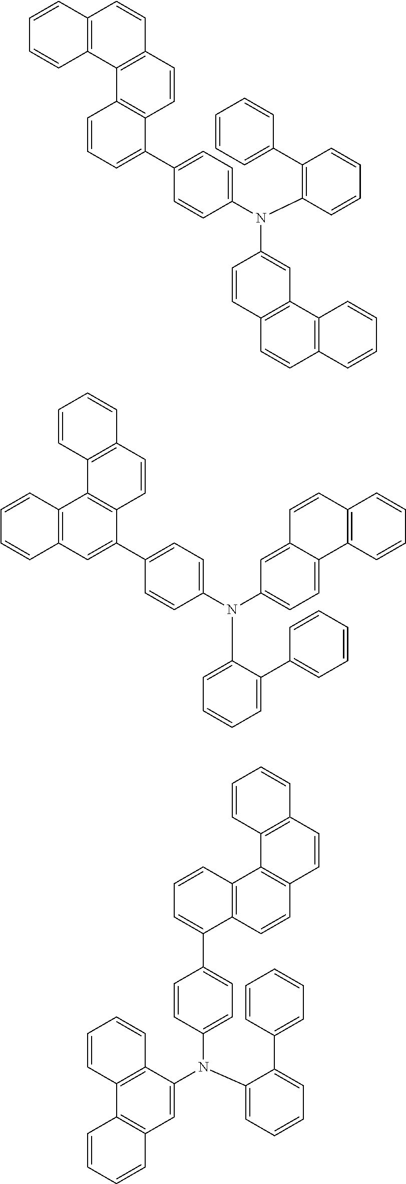 [SMILES: c1ccc(-c2ccccc2N(c2ccc(-c3cccc4c3ccc3ccc5ccccc5c34)cc2)c2ccc3ccc4ccccc4c3c2)cc1 ; c1ccc(-c2ccccc2N(c2ccc(-c3cc4ccccc4c4c3ccc3ccccc34)cc2)c2ccc3c(ccc4ccccc43)c2)cc1 ; c1ccc(-c2ccccc2N(c2ccc(-c3cccc4c3ccc3ccc5ccccc5c34)cc2)c2cc3ccccc3c3ccccc23)cc1]

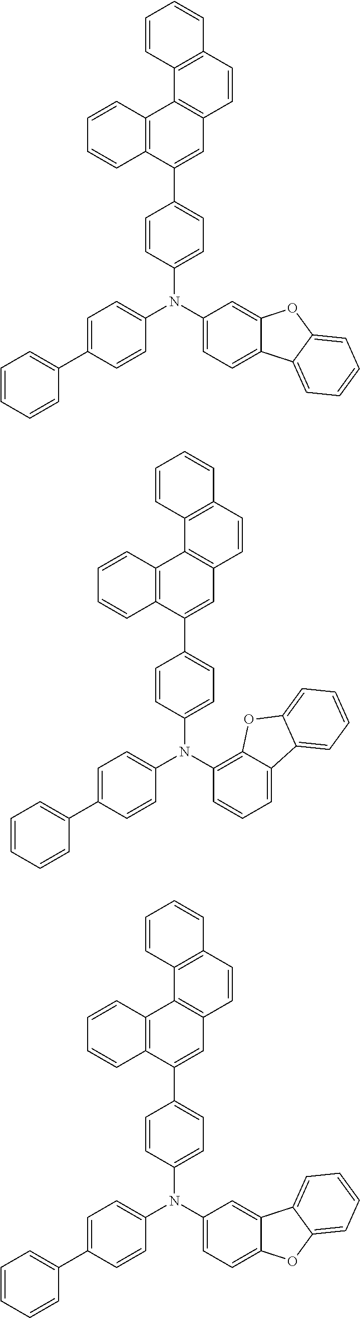 [SMILES: c1ccc(-c2ccc(N(c3ccc(-c4cc5ccc6ccccc6c5c5ccccc45)cc3)c3ccc4c(c3)oc3ccccc34)cc2)cc1 ; c1ccc(-c2ccc(N(c3ccc(-c4cc5ccc6ccccc6c5c5ccccc45)cc3)c3cccc4c3oc3ccccc34)cc2)cc1 ; c1ccc(-c2ccc(N(c3ccc(-c4cc5ccc6ccccc6c5c5ccccc45)cc3)c3ccc4oc5ccccc5c4c3)cc2)cc1]

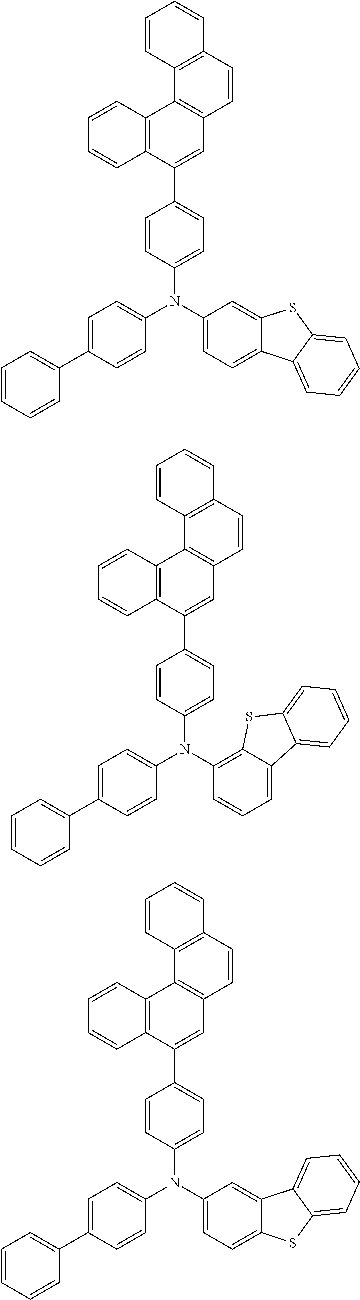 [SMILES: c1ccc(-c2ccc(N(c3ccc(-c4cc5ccc6ccccc6c5c5ccccc45)cc3)c3ccc4c(c3)sc3ccccc34)cc2)cc1 ; c1ccc(-c2ccc(N(c3ccc(-c4cc5ccc6ccccc6c5c5ccccc45)cc3)c3cccc4c3sc3ccccc34)cc2)cc1 ; c1ccc(-c2ccc(N(c3ccc(-c4cc5ccc6ccccc6c5c5ccccc45)cc3)c3ccc4sc5ccccc5c4c3)cc2)cc1]

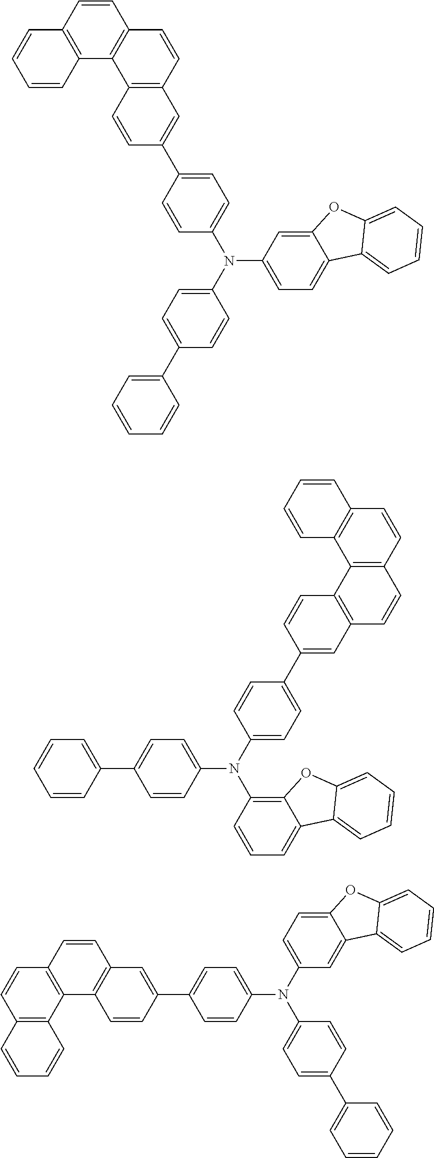 [SMILES: c1ccc(-c2ccc(N(c3ccc(-c4ccc5c(ccc6ccc7ccccc7c65)c4)cc3)c3ccc4c(c3)oc3ccccc34)cc2)cc1 ; c1ccc(-c2ccc(N(c3ccc(-c4ccc5c(ccc6ccc7ccccc7c65)c4)cc3)c3cccc4c3oc3ccccc34)cc2)cc1 ; c1ccc(-c2ccc(N(c3ccc(-c4ccc5c(ccc6ccc7ccccc7c65)c4)cc3)c3ccc4oc5ccccc5c4c3)cc2)cc1]

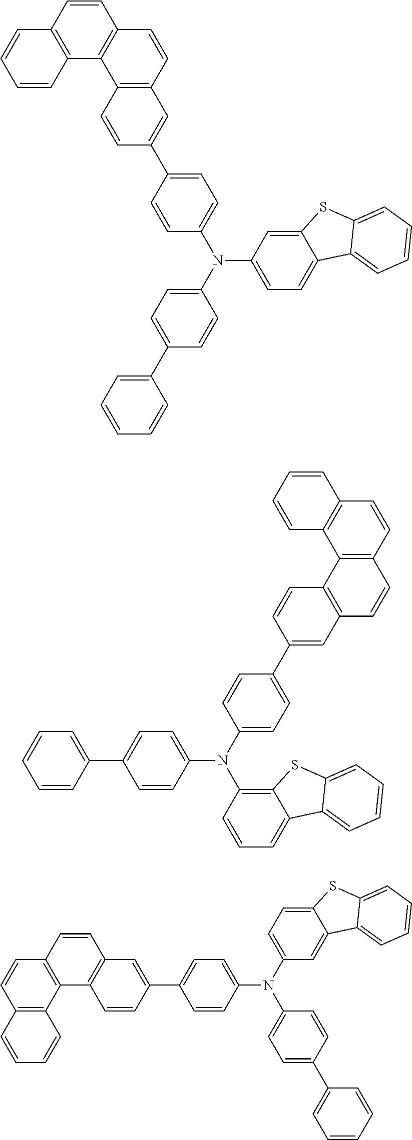 [SMILES: c1ccc(-c2ccc(N(c3ccc(-c4ccc5c(ccc6ccc7ccccc7c65)c4)cc3)c3ccc4c(c3)sc3ccccc34)cc2)cc1 ; c1ccc(-c2ccc(N(c3ccc(-c4ccc5c(ccc6ccc7ccccc7c65)c4)cc3)c3cccc4c3sc3ccccc34)cc2)cc1 ; c1ccc(-c2ccc(N(c3ccc(-c4ccc5c(ccc6ccc7ccccc7c65)c4)cc3)c3ccc4sc5ccccc5c4c3)cc2)cc1]

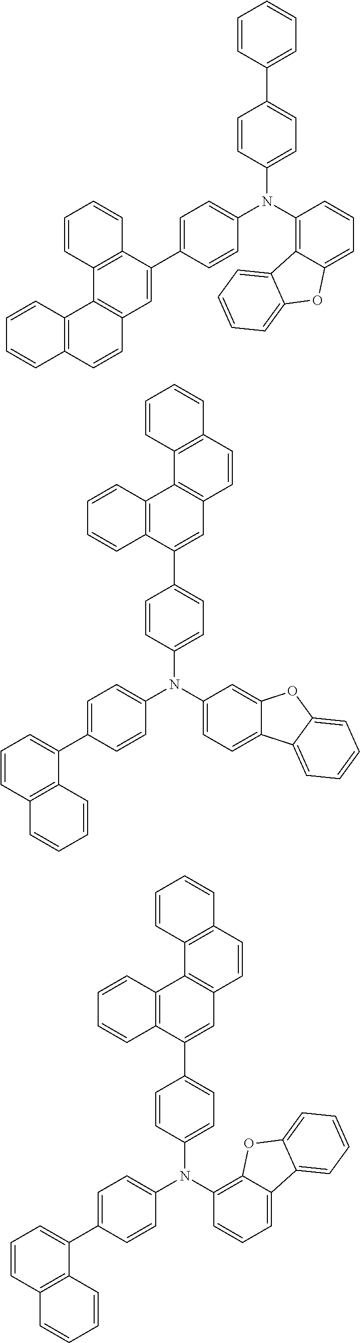 [SMILES: c1ccc(-c2ccc(N(c3ccc(-c4cc5ccc6ccccc6c5c5ccccc45)cc3)c3cccc4oc5ccccc5c34)cc2)cc1 ; c1ccc2c(-c3ccc(N(c4ccc(-c5cc6ccc7ccccc7c6c6ccccc56)cc4)c4ccc5c(c4)oc4ccccc45)cc3)cccc2c1 ; c1ccc2c(-c3ccc(N(c4ccc(-c5cc6ccc7ccccc7c6c6ccccc56)cc4)c4cccc5c4oc4ccccc45)cc3)cccc2c1]

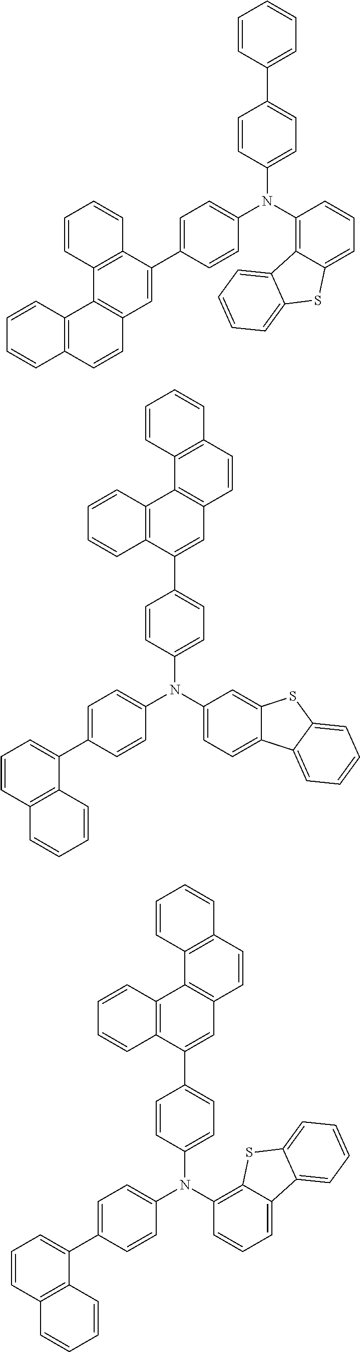 [SMILES: c1ccc(-c2ccc(N(c3ccc(-c4cc5ccc6ccccc6c5c5ccccc45)cc3)c3cccc4sc5ccccc5c34)cc2)cc1 ; c1ccc2c(-c3ccc(N(c4ccc(-c5cc6ccc7ccccc7c6c6ccccc56)cc4)c4ccc5c(c4)sc4ccccc45)cc3)cccc2c1 ; c1ccc2c(-c3ccc(N(c4ccc(-c5cc6ccc7ccccc7c6c6ccccc56)cc4)c4cccc5c4sc4ccccc45)cc3)cccc2c1]

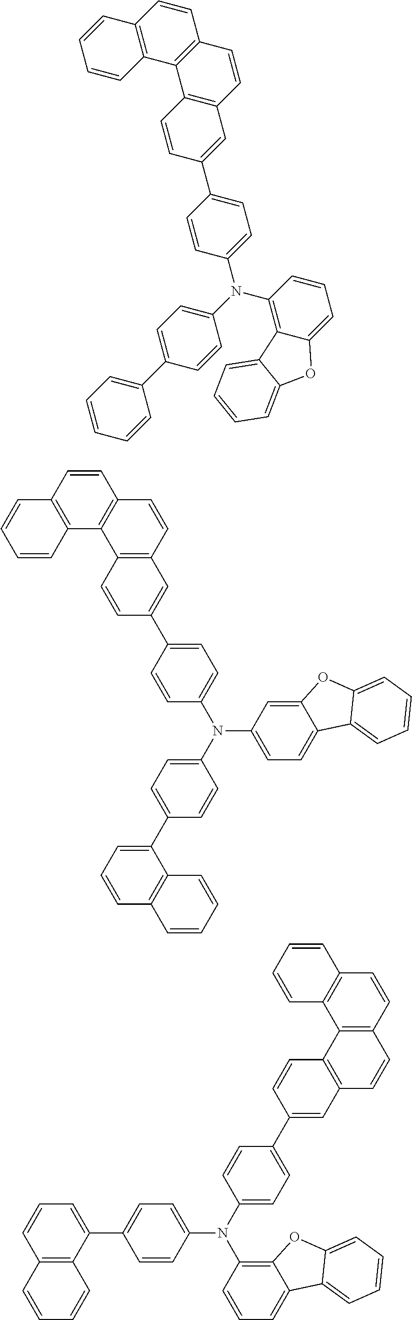

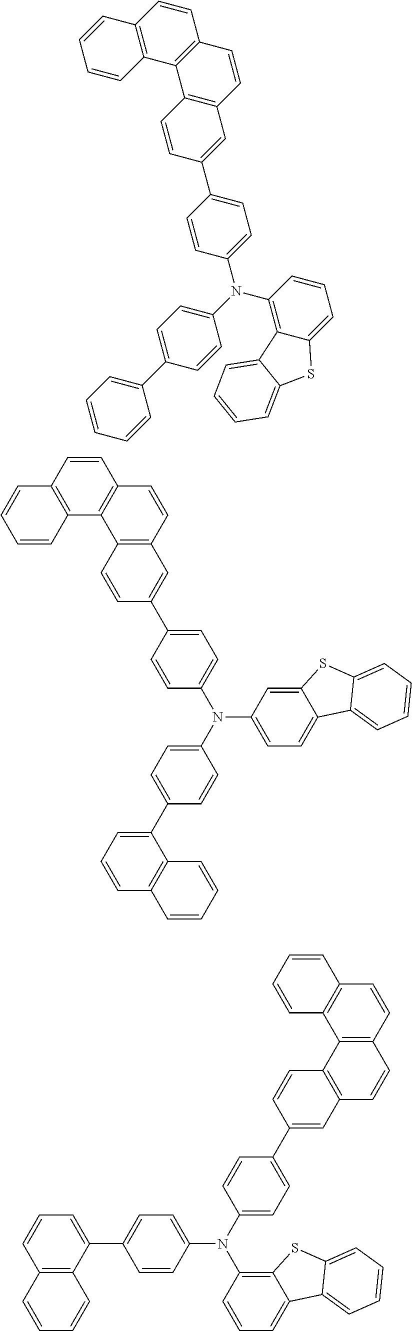

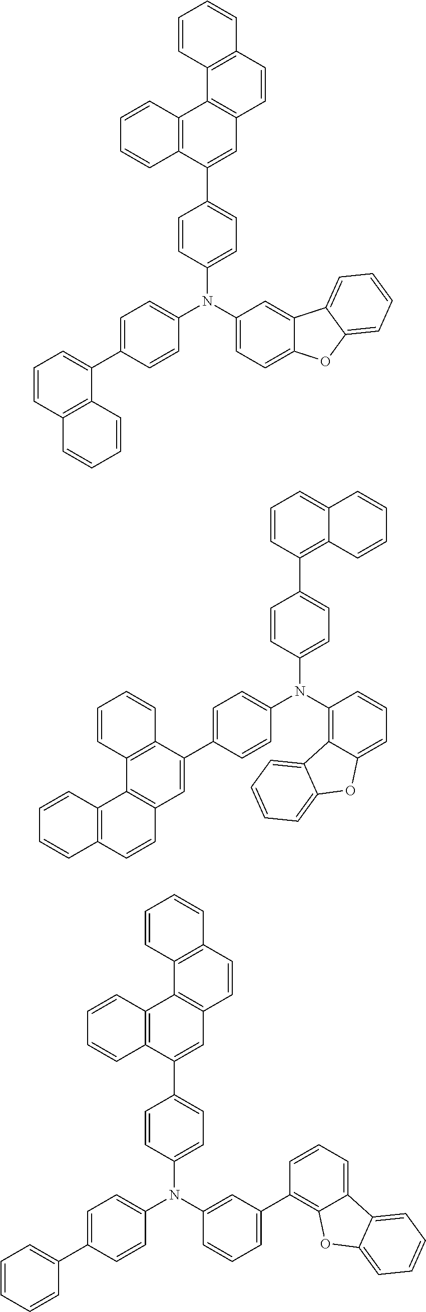 [SMILES: c1ccc2c(-c3ccc(N(c4ccc(-c5cc6ccc7ccccc7c6c6ccccc56)cc4)c4ccc5oc6ccccc6c5c4)cc3)cccc2c1 ; c1ccc2c(-c3ccc(N(c4ccc(-c5cc6ccc7ccccc7c6c6ccccc56)cc4)c4cccc5oc6ccccc6c45)cc3)cccc2c1 ; c1ccc(-c2ccc(N(c3ccc(-c4cc5ccc6ccccc6c5c5ccccc45)cc3)c3cccc(-c4cccc5c4oc4ccccc45)c3)cc2)cc1]

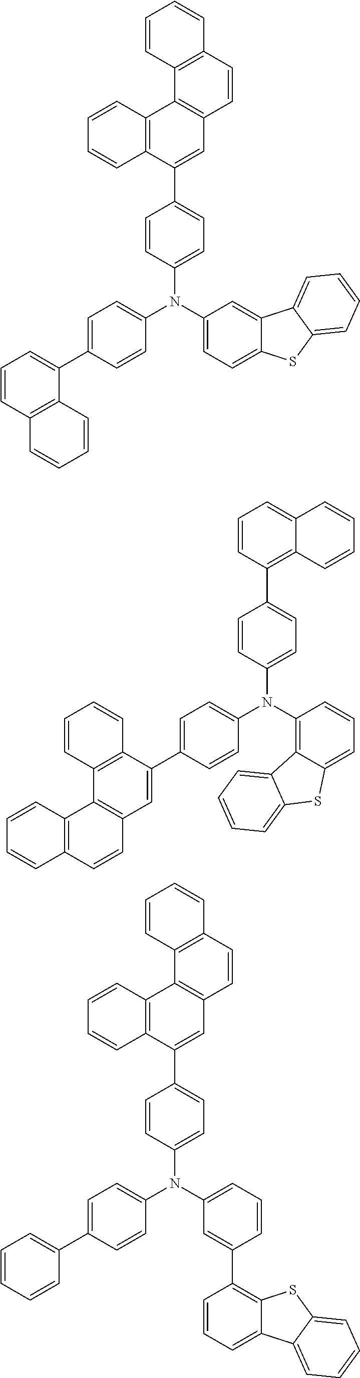 [SMILES: c1ccc2c(-c3ccc(N(c4ccc(-c5cc6ccc7ccccc7c6c6ccccc56)cc4)c4ccc5sc6ccccc6c5c4)cc3)cccc2c1 ; c1ccc2c(-c3ccc(N(c4ccc(-c5cc6ccc7ccccc7c6c6ccccc56)cc4)c4cccc5sc6ccccc6c45)cc3)cccc2c1 ; c1ccc(-c2ccc(N(c3ccc(-c4cc5ccc6ccccc6c5c5ccccc45)cc3)c3cccc(-c4cccc5c4sc4ccccc45)c3)cc2)cc1]

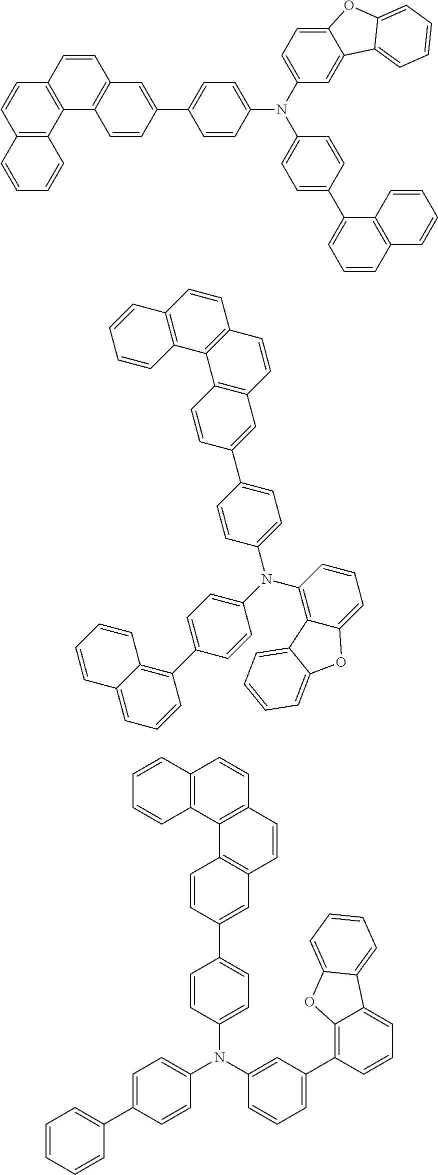

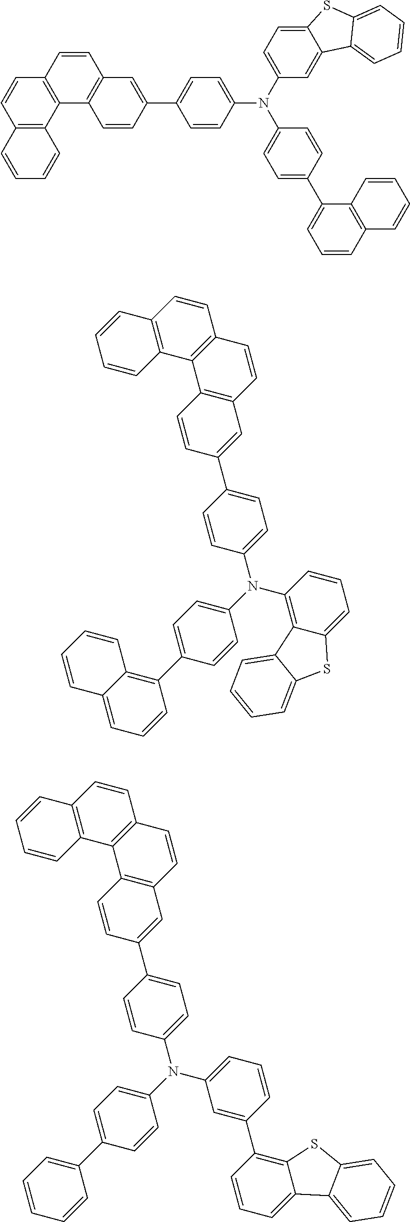 [SMILES: c1ccc2c(-c3ccc(N(c4ccc(-c5ccc6c(ccc7ccc8ccccc8c76)c5)cc4)c4ccc5sc6ccccc6c5c4)cc3)cccc2c1 ; c1ccc2c(-c3ccc(N(c4ccc(-c5ccc6c(ccc7ccc8ccccc8c76)c5)cc4)c4cccc5sc6ccccc6c45)cc3)cccc2c1 ; c1ccc(-c2ccc(N(c3ccc(-c4ccc5c(ccc6ccc7ccccc7c65)c4)cc3)c3cccc(-c4cccc5c4sc4ccccc45)c3)cc2)cc1]

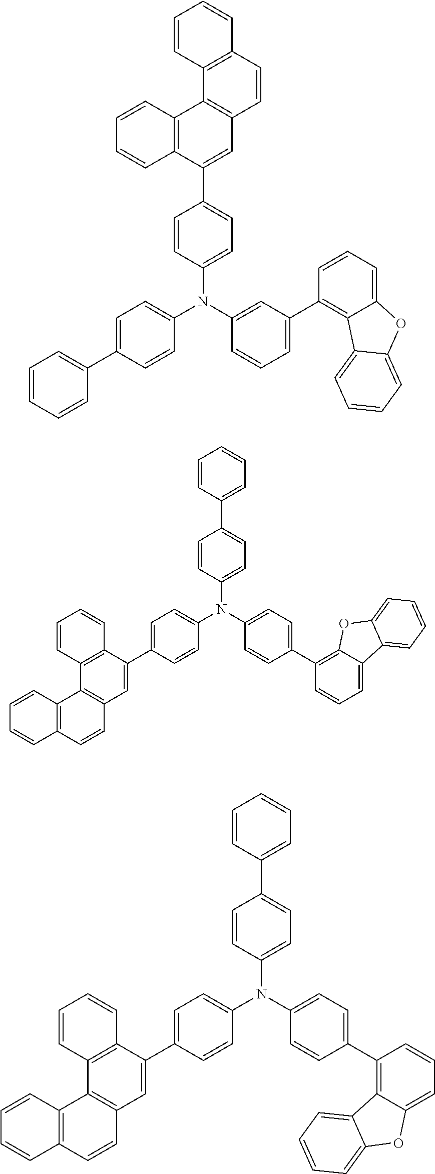 [SMILES: c1ccc(-c2ccc(N(c3ccc(-c4cc5ccc6ccccc6c5c5ccccc45)cc3)c3cccc(-c4cccc5oc6ccccc6c45)c3)cc2)cc1 ; c1ccc(-c2ccc(N(c3ccc(-c4cc5ccc6ccccc6c5c5ccccc45)cc3)c3ccc(-c4cccc5c4oc4ccccc45)cc3)cc2)cc1 ; c1ccc(-c2ccc(N(c3ccc(-c4cc5ccc6ccccc6c5c5ccccc45)cc3)c3ccc(-c4cccc5oc6ccccc6c45)cc3)cc2)cc1]

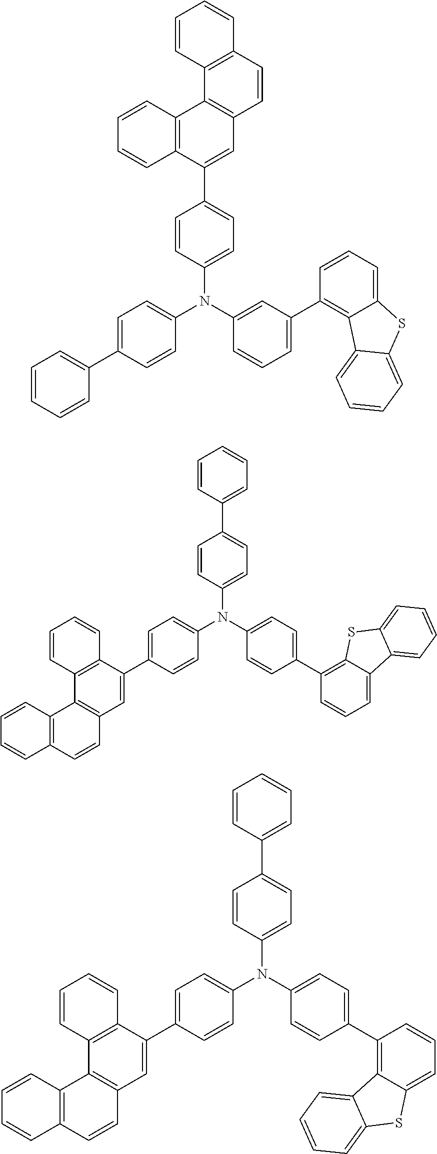 [SMILES: c1ccc(-c2ccc(N(c3ccc(-c4cc5ccc6ccccc6c5c5ccccc45)cc3)c3cccc(-c4cccc5sc6ccccc6c45)c3)cc2)cc1 ; c1ccc(-c2ccc(N(c3ccc(-c4cc5ccc6ccccc6c5c5ccccc45)cc3)c3ccc(-c4cccc5c4sc4ccccc45)cc3)cc2)cc1 ; c1ccc(-c2ccc(N(c3ccc(-c4cc5ccc6ccccc6c5c5ccccc45)cc3)c3ccc(-c4cccc5sc6ccccc6c45)cc3)cc2)cc1]

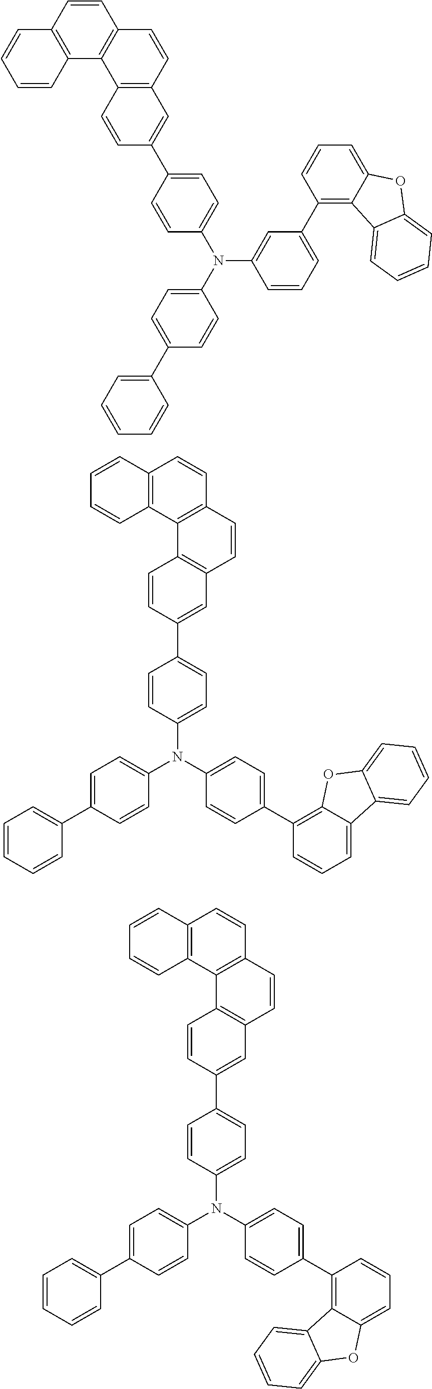

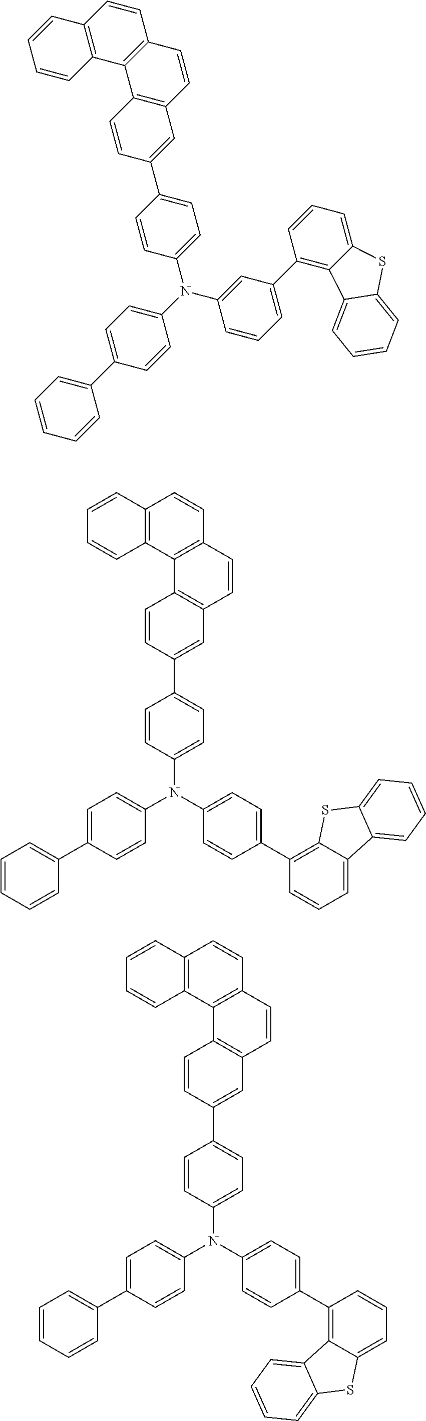 [SMILES: c1ccc(-c2ccc(N(c3ccc(-c4ccc5c(ccc6ccc7ccccc7c65)c4)cc3)c3cccc(-c4cccc5sc6ccccc6c45)c3)cc2)cc1 ; c1ccc(-c2ccc(N(c3ccc(-c4ccc5c(ccc6ccc7ccccc7c65)c4)cc3)c3ccc(-c4cccc5c4sc4ccccc45)cc3)cc2)cc1 ; c1ccc(-c2ccc(N(c3ccc(-c4ccc5c(ccc6ccc7ccccc7c65)c4)cc3)c3ccc(-c4cccc5sc6ccccc6c45)cc3)cc2)cc1]

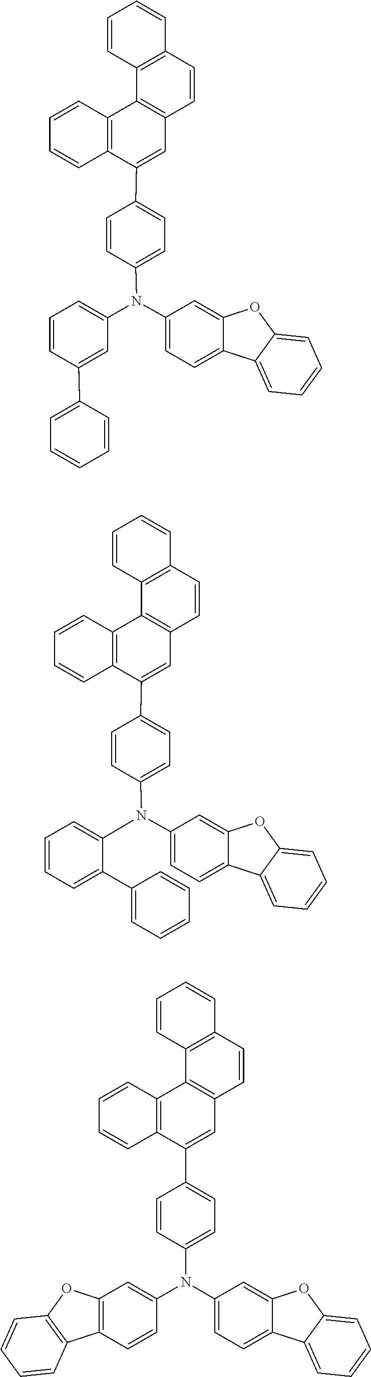 [SMILES: c1ccc(-c2cccc(N(c3ccc(-c4cc5ccc6ccccc6c5c5ccccc45)cc3)c3ccc4c(c3)oc3ccccc34)c2)cc1 ; c1ccc(-c2ccccc2N(c2ccc(-c3cc4ccc5ccccc5c4c4ccccc34)cc2)c2ccc3c(c2)oc2ccccc23)cc1 ; c1ccc2c(c1)ccc1cc(-c3ccc(N(c4ccc5c(c4)oc4ccccc45)c4ccc5c(c4)oc4ccccc45)cc3)c3ccccc3c12]

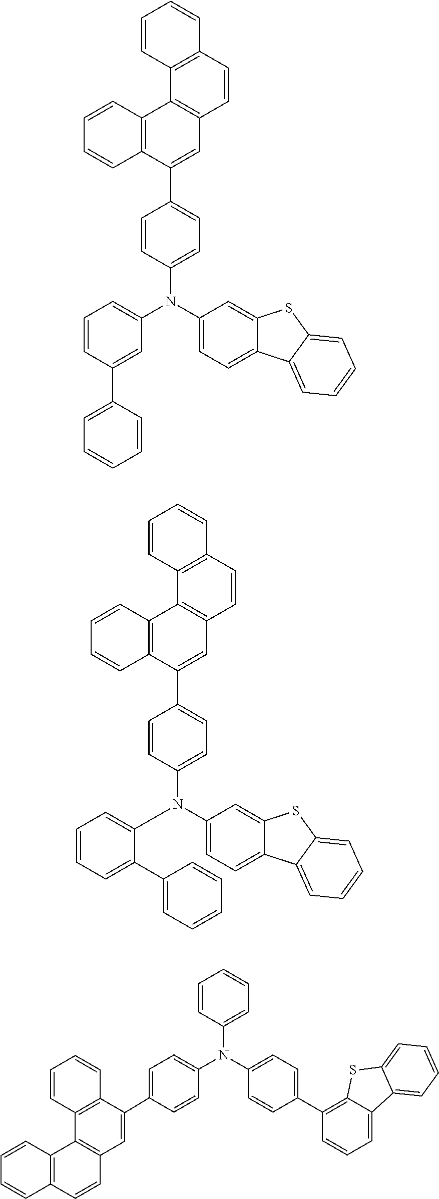 [SMILES: c1ccc(-c2cccc(N(c3ccc(-c4cc5ccc6ccccc6c5c5ccccc45)cc3)c3ccc4c(c3)sc3ccccc34)c2)cc1 ; c1ccc(-c2ccccc2N(c2ccc(-c3cc4ccc5ccccc5c4c4ccccc34)cc2)c2ccc3c(c2)sc2ccccc23)cc1 ; c1ccc(N(c2ccc(-c3cc4ccc5ccccc5c4c4ccccc34)cc2)c2ccc(-c3cccc4c3sc3ccccc34)cc2)cc1]

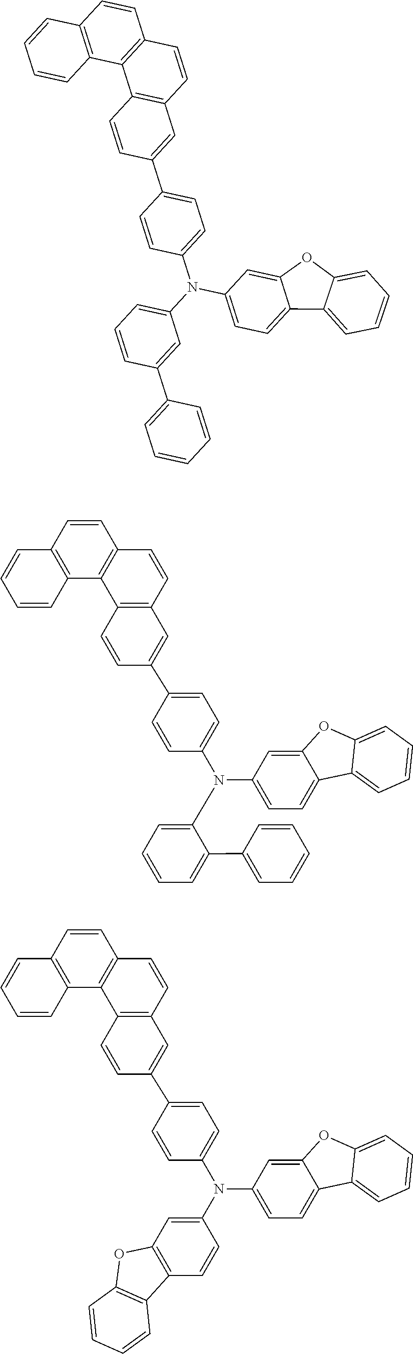 [SMILES: c1ccc(-c2cccc(N(c3ccc(-c4ccc5c(ccc6ccc7ccccc7c65)c4)cc3)c3ccc4c(c3)oc3ccccc34)c2)cc1 ; c1ccc(-c2ccccc2N(c2ccc(-c3ccc4c(ccc5ccc6ccccc6c54)c3)cc2)c2ccc3c(c2)oc2ccccc23)cc1 ; c1ccc2c(c1)ccc1ccc3cc(-c4ccc(N(c5ccc6c(c5)oc5ccccc56)c5ccc6c(c5)oc5ccccc56)cc4)ccc3c12]

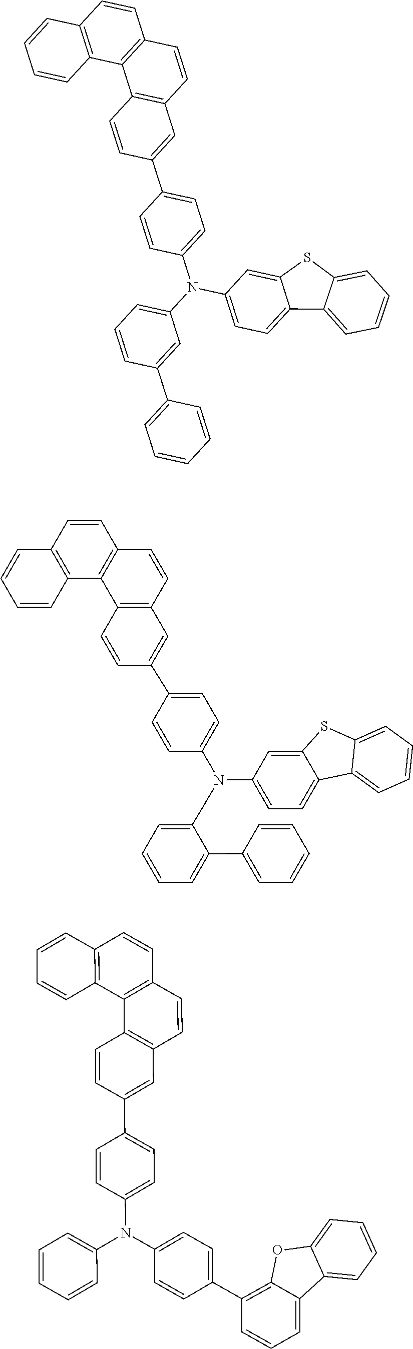 [SMILES: c1ccc(-c2cccc(N(c3ccc(-c4ccc5c(ccc6ccc7ccccc7c65)c4)cc3)c3ccc4c(c3)sc3ccccc34)c2)cc1 ; c1ccc(-c2ccccc2N(c2ccc(-c3ccc4c(ccc5ccc6ccccc6c54)c3)cc2)c2ccc3c(c2)sc2ccccc23)cc1 ; c1ccc(N(c2ccc(-c3ccc4c(ccc5ccc6ccccc6c54)c3)cc2)c2ccc(-c3cccc4c3oc3ccccc34)cc2)cc1]

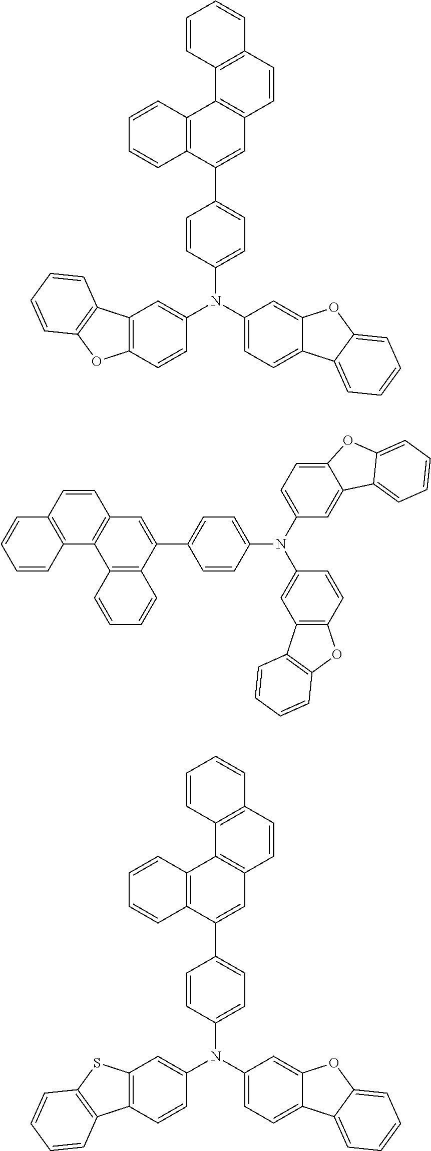 [SMILES: c1ccc2c(c1)ccc1cc(-c3ccc(N(c4ccc5c(c4)oc4ccccc45)c4ccc5oc6ccccc6c5c4)cc3)c3ccccc3c12 ; c1ccc2c(c1)ccc1cc(-c3ccc(N(c4ccc5oc6ccccc6c5c4)c4ccc5oc6ccccc6c5c4)cc3)c3ccccc3c12 ; c1ccc2c(c1)ccc1cc(-c3ccc(N(c4ccc5c(c4)oc4ccccc45)c4ccc5c(c4)sc4ccccc45)cc3)c3ccccc3c12]

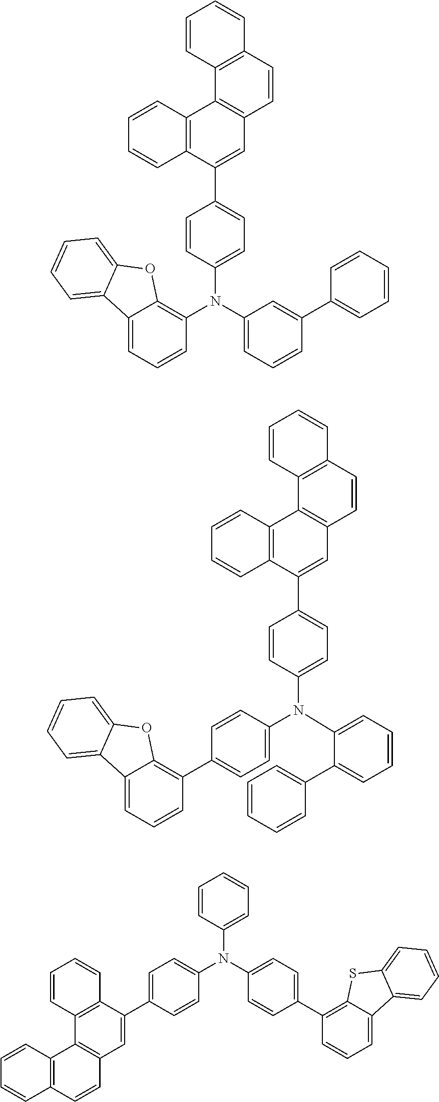 [SMILES: c1ccc(-c2cccc(N(c3ccc(-c4cc5ccc6ccccc6c5c5ccccc45)cc3)c3cccc4c3oc3ccccc34)c2)cc1 ; c1ccc(-c2ccccc2N(c2ccc(-c3cc4ccc5ccccc5c4c4ccccc34)cc2)c2ccc(-c3cccc4c3oc3ccccc34)cc2)cc1 ; c1ccc(N(c2ccc(-c3cc4ccc5ccccc5c4c4ccccc34)cc2)c2ccc(-c3cccc4c3sc3ccccc34)cc2)cc1]

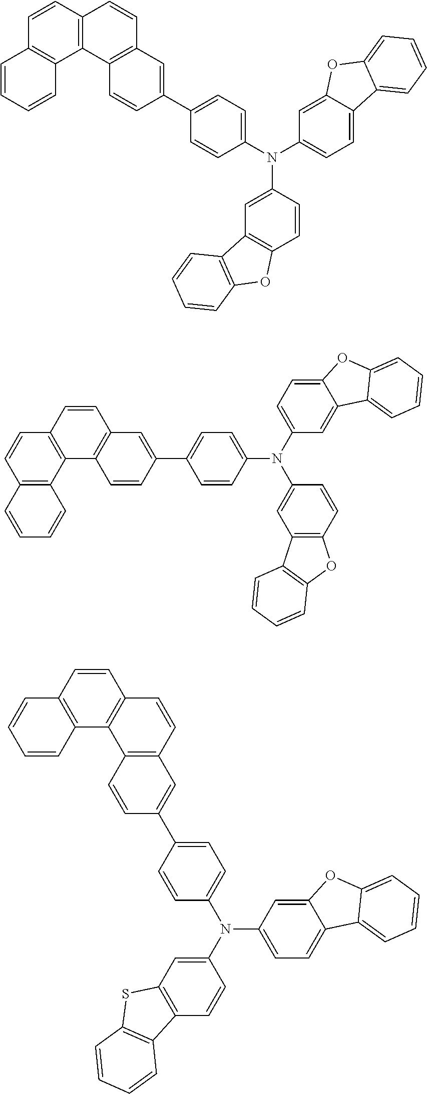 [SMILES: c1ccc2c(c1)ccc1ccc3cc(-c4ccc(N(c5ccc6c(c5)oc5ccccc56)c5ccc6oc7ccccc7c6c5)cc4)ccc3c12 ; c1ccc2c(c1)ccc1ccc3cc(-c4ccc(N(c5ccc6oc7ccccc7c6c5)c5ccc6oc7ccccc7c6c5)cc4)ccc3c12 ; c1ccc2c(c1)ccc1ccc3cc(-c4ccc(N(c5ccc6c(c5)oc5ccccc56)c5ccc6c(c5)sc5ccccc56)cc4)ccc3c12]

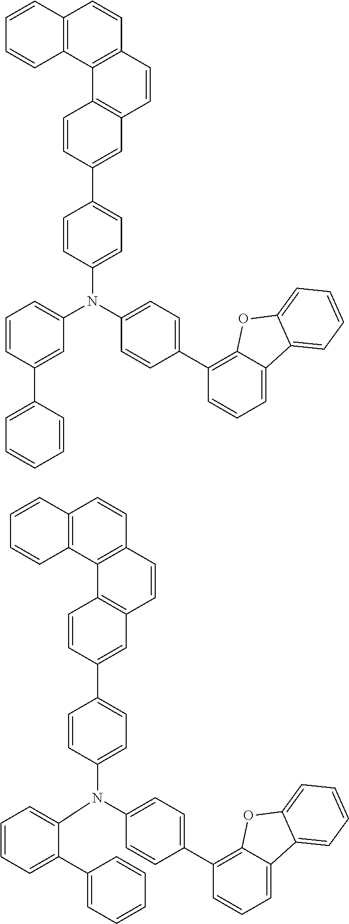

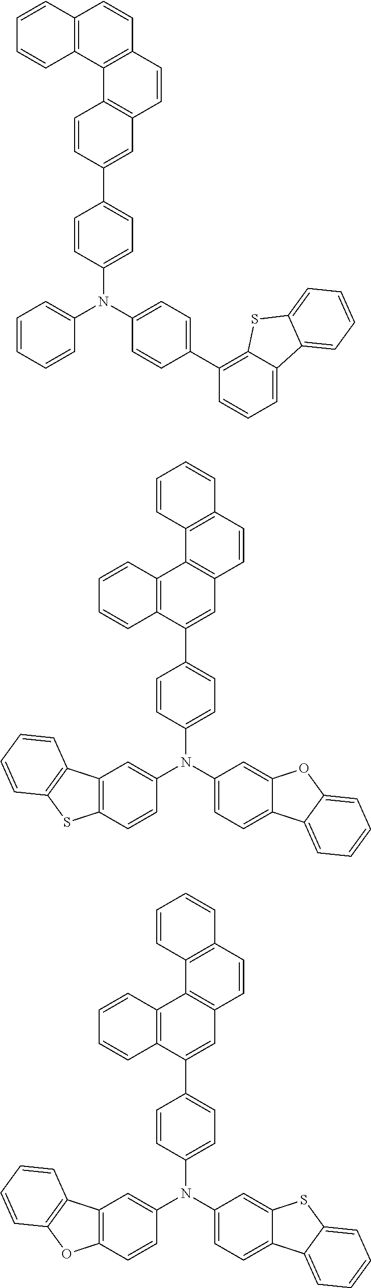 [SMILES: c1ccc(N(c2ccc(-c3ccc4c(ccc5ccc6ccccc6c54)c3)cc2)c2ccc(-c3cccc4c3sc3ccccc34)cc2)cc1 ; c1ccc2c(c1)ccc1cc(-c3ccc(N(c4ccc5c(c4)oc4ccccc45)c4ccc5sc6ccccc6c5c4)cc3)c3ccccc3c12 ; c1ccc2c(c1)ccc1cc(-c3ccc(N(c4ccc5c(c4)sc4ccccc45)c4ccc5oc6ccccc6c5c4)cc3)c3ccccc3c12]

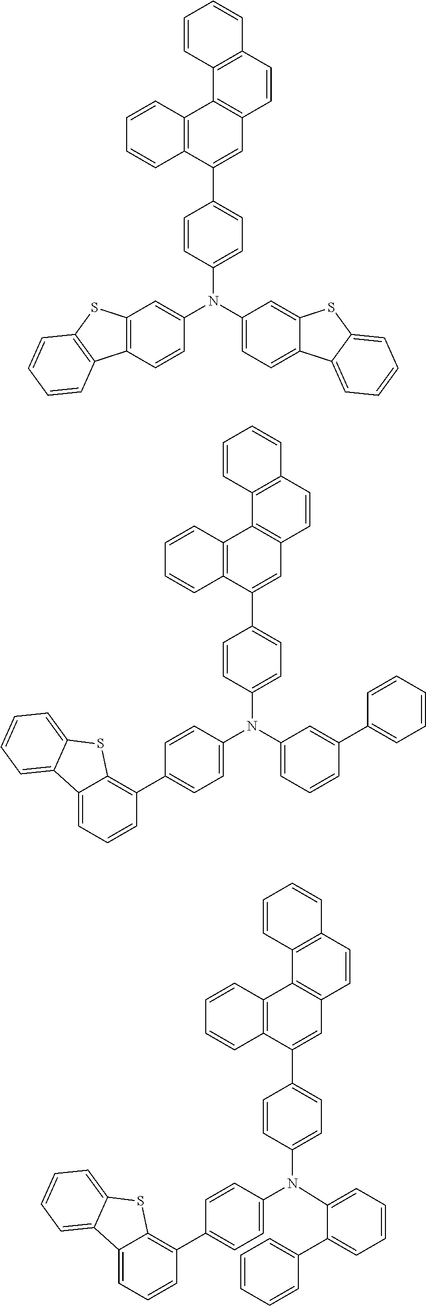 [SMILES: c1ccc2c(c1)ccc1cc(-c3ccc(N(c4ccc5c(c4)sc4ccccc45)c4ccc5c(c4)sc4ccccc45)cc3)c3ccccc3c12 ; c1ccc(-c2cccc(N(c3ccc(-c4cc5ccc6ccccc6c5c5ccccc45)cc3)c3ccc(-c4cccc5c4sc4ccccc45)cc3)c2)cc1 ; c1ccc(-c2ccccc2N(c2ccc(-c3cc4ccc5ccccc5c4c4ccccc34)cc2)c2ccc(-c3cccc4c3sc3ccccc34)cc2)cc1]

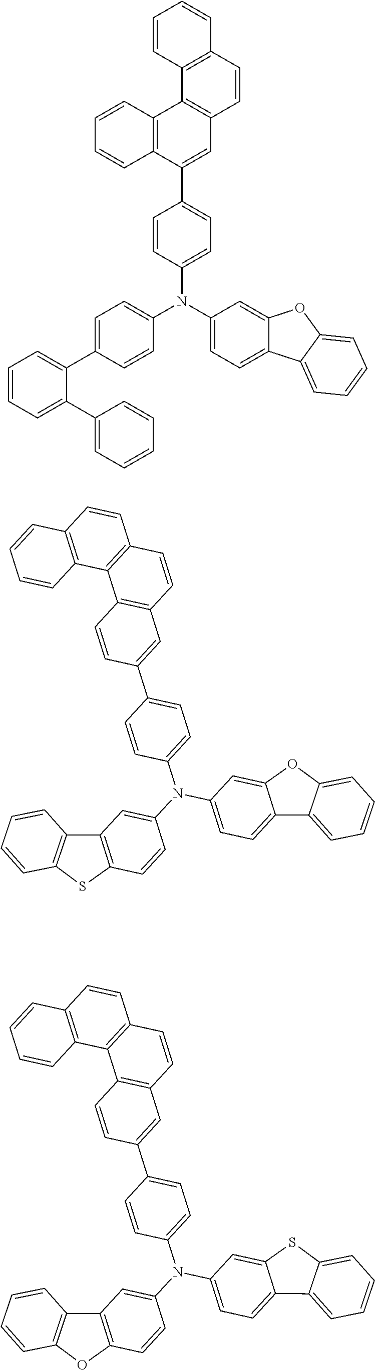

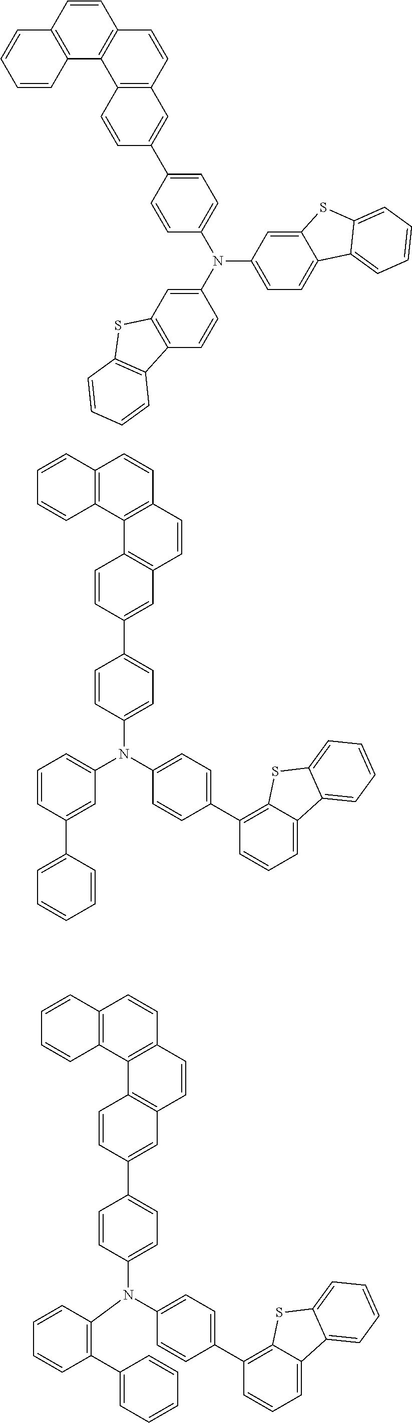

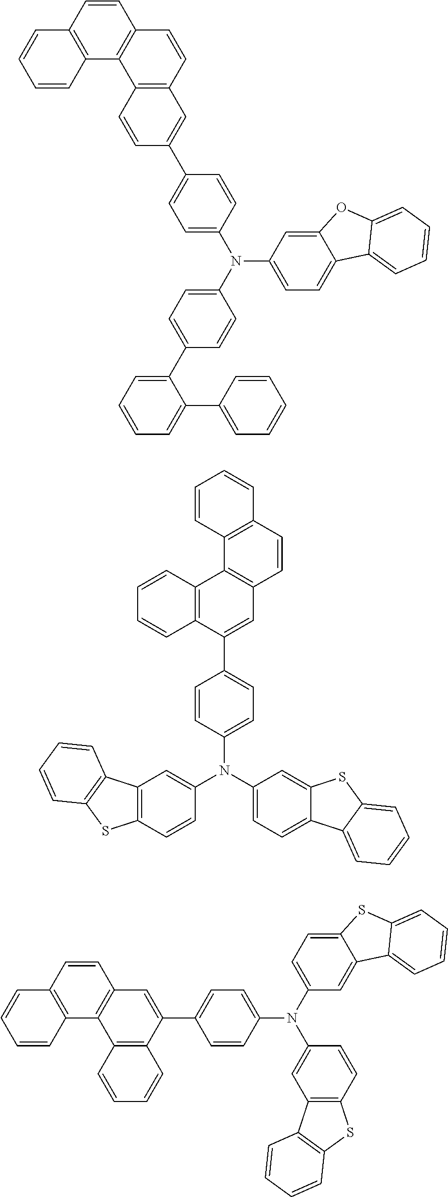

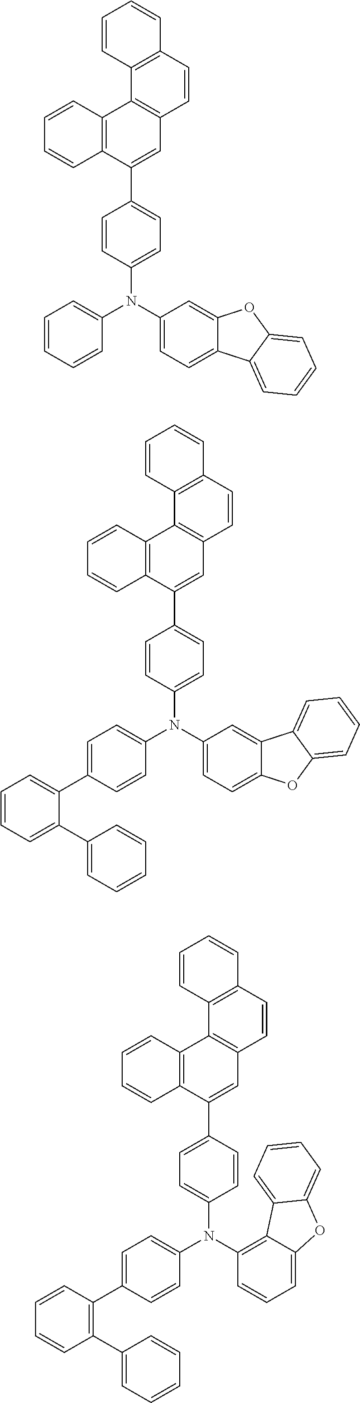 [SMILES: c1ccc(N(c2ccc(-c3cc4ccc5ccccc5c4c4ccccc34)cc2)c2ccc3c(c2)oc2ccccc23)cc1 ; c1ccc(-c2ccccc2-c2ccc(N(c3ccc(-c4cc5ccc6ccccc6c5c5ccccc45)cc3)c3ccc4oc5ccccc5c4c3)cc2)cc1 ; c1ccc(-c2ccccc2-c2ccc(N(c3ccc(-c4cc5ccc6ccccc6c5c5ccccc45)cc3)c3cccc4oc5ccccc5c34)cc2)cc1]

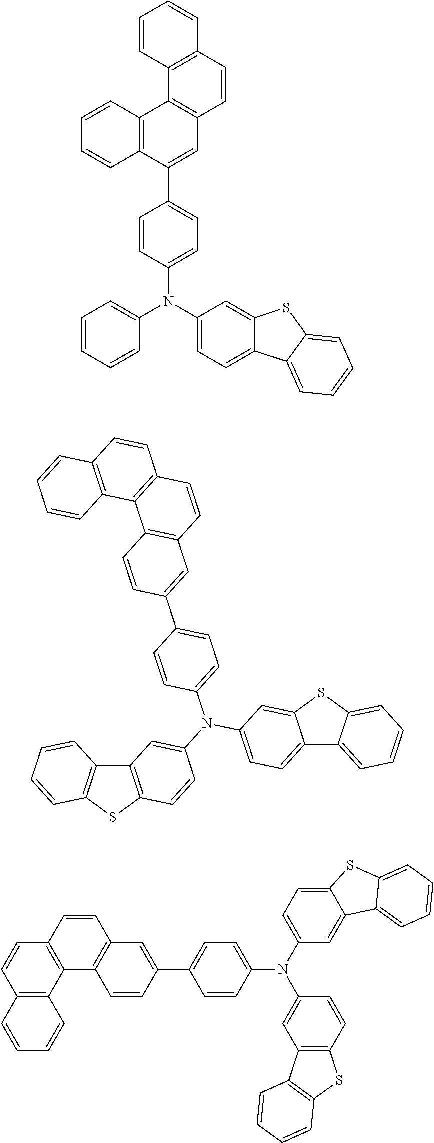 [SMILES: c1ccc(N(c2ccc(-c3cc4ccc5ccccc5c4c4ccccc34)cc2)c2ccc3c(c2)sc2ccccc23)cc1 ; c1ccc2c(c1)ccc1ccc3cc(-c4ccc(N(c5ccc6c(c5)sc5ccccc56)c5ccc6sc7ccccc7c6c5)cc4)ccc3c12 ; c1ccc2c(c1)ccc1ccc3cc(-c4ccc(N(c5ccc6sc7ccccc7c6c5)c5ccc6sc7ccccc7c6c5)cc4)ccc3c12]

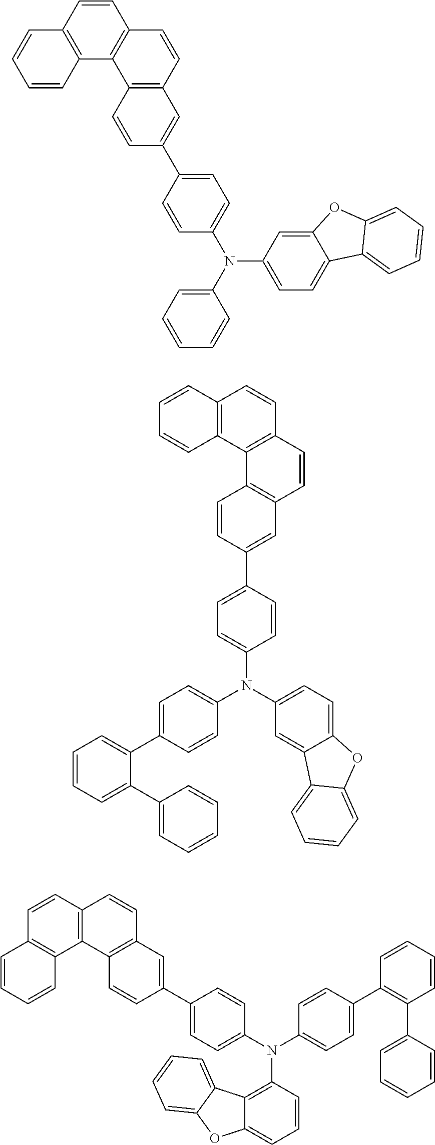

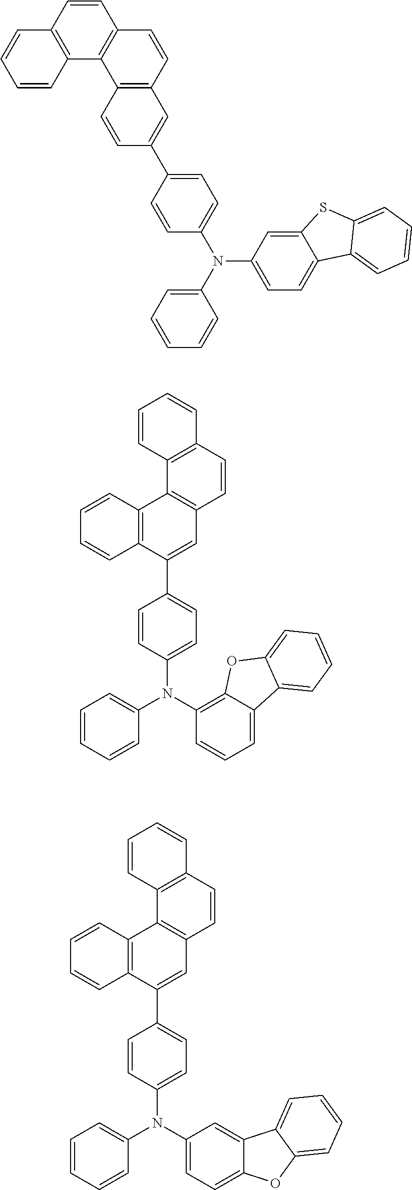

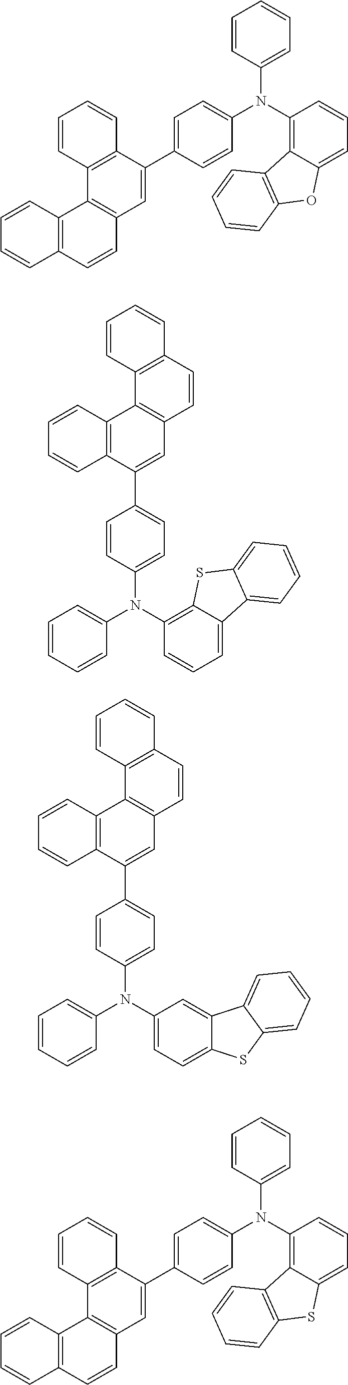 [SMILES: c1ccc(N(c2ccc(-c3cc4ccc5ccccc5c4c4ccccc34)cc2)c2cccc3oc4ccccc4c23)cc1 ; c1ccc(N(c2ccc(-c3cc4ccc5ccccc5c4c4ccccc34)cc2)c2cccc3c2sc2ccccc23)cc1 ; c1ccc(N(c2ccc(-c3cc4ccc5ccccc5c4c4ccccc34)cc2)c2ccc3sc4ccccc4c3c2)cc1 ; c1ccc(N(c2ccc(-c3cc4ccc5ccccc5c4c4ccccc34)cc2)c2cccc3sc4ccccc4c23)cc1]

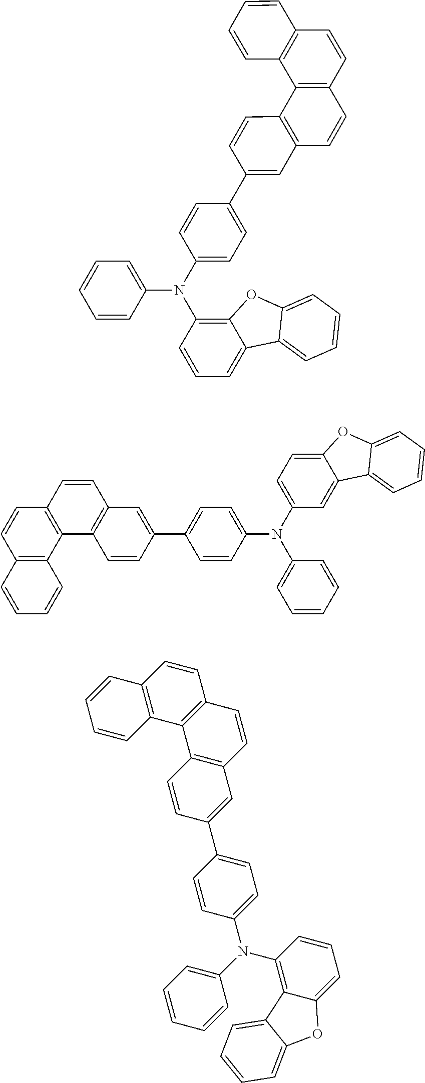 [SMILES: c1ccc(N(c2ccc(-c3ccc4c(ccc5ccc6ccccc6c54)c3)cc2)c2cccc3c2oc2ccccc23)cc1 ; c1ccc(N(c2ccc(-c3ccc4c(ccc5ccc6ccccc6c54)c3)cc2)c2ccc3oc4ccccc4c3c2)cc1 ; c1ccc(N(c2ccc(-c3ccc4c(ccc5ccc6ccccc6c54)c3)cc2)c2cccc3oc4ccccc4c23)cc1]

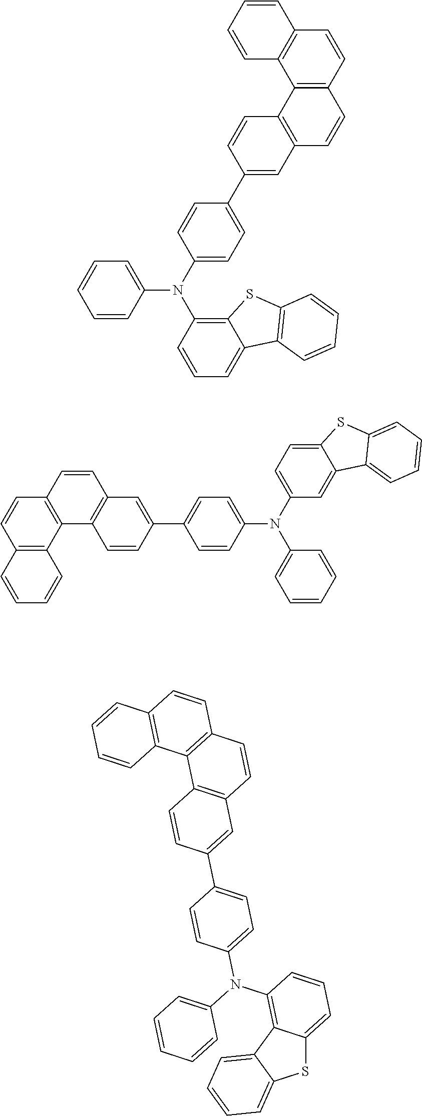 [SMILES: c1ccc(N(c2ccc(-c3ccc4c(ccc5ccc6ccccc6c54)c3)cc2)c2cccc3c2sc2ccccc23)cc1 ; c1ccc(N(c2ccc(-c3ccc4c(ccc5ccc6ccccc6c54)c3)cc2)c2ccc3sc4ccccc4c3c2)cc1 ; c1ccc(N(c2ccc(-c3ccc4c(ccc5ccc6ccccc6c54)c3)cc2)c2cccc3sc4ccccc4c23)cc1]

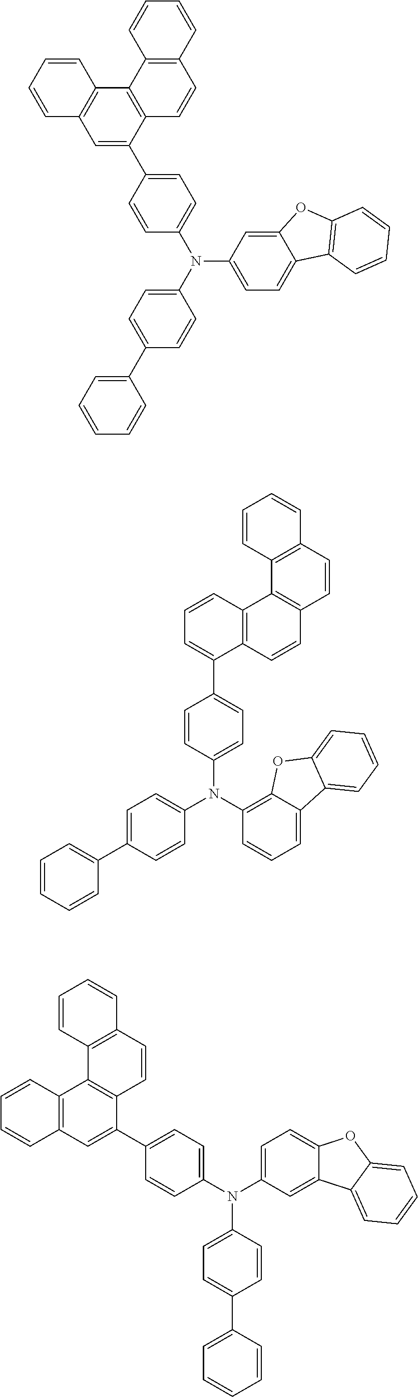

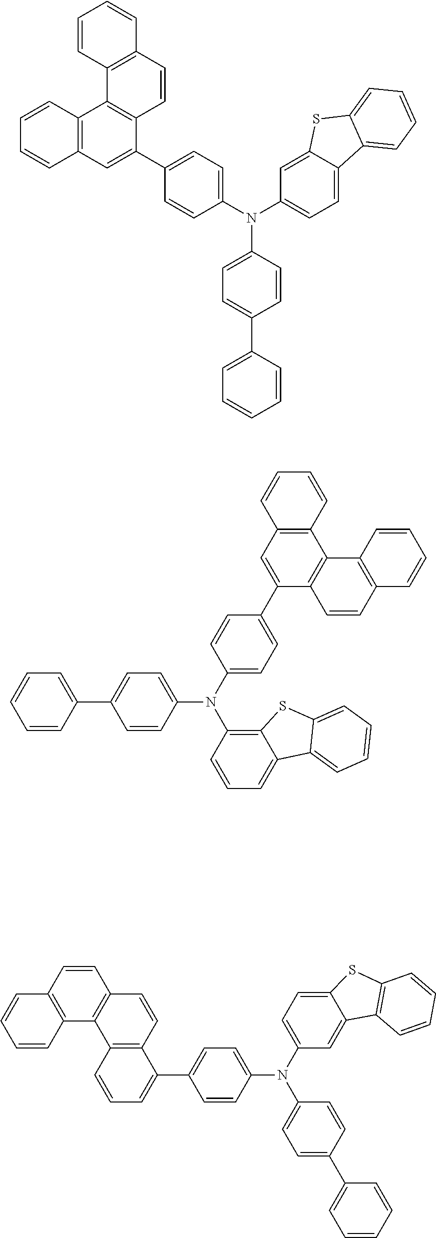

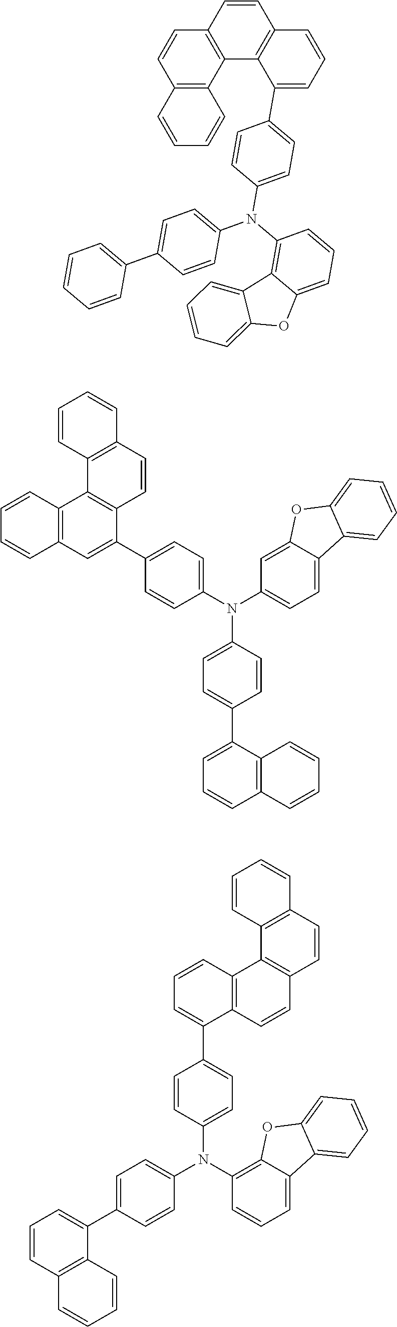 [SMILES: c1ccc(-c2ccc(N(c3ccc(-c4cccc5ccc6ccc7ccccc7c6c45)cc3)c3cccc4oc5ccccc5c34)cc2)cc1 ; c1ccc2c(-c3ccc(N(c4ccc(-c5cc6ccccc6c6c5ccc5ccccc56)cc4)c4ccc5c(c4)oc4ccccc45)cc3)cccc2c1 ; c1ccc2c(-c3ccc(N(c4ccc(-c5cccc6c5ccc5ccc7ccccc7c56)cc4)c4cccc5c4oc4ccccc45)cc3)cccc2c1]

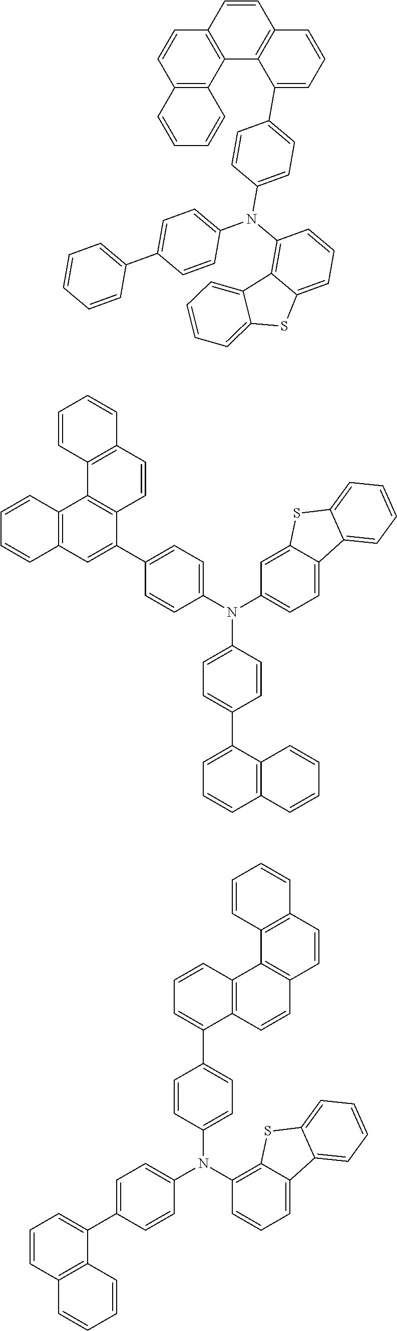 [SMILES: c1ccc(-c2ccc(N(c3ccc(-c4cccc5ccc6ccc7ccccc7c6c45)cc3)c3cccc4sc5ccccc5c34)cc2)cc1 ; c1ccc2c(-c3ccc(N(c4ccc(-c5cc6ccccc6c6c5ccc5ccccc56)cc4)c4ccc5c(c4)sc4ccccc45)cc3)cccc2c1 ; c1ccc2c(-c3ccc(N(c4ccc(-c5cccc6c5ccc5ccc7ccccc7c56)cc4)c4cccc5c4sc4ccccc45)cc3)cccc2c1]

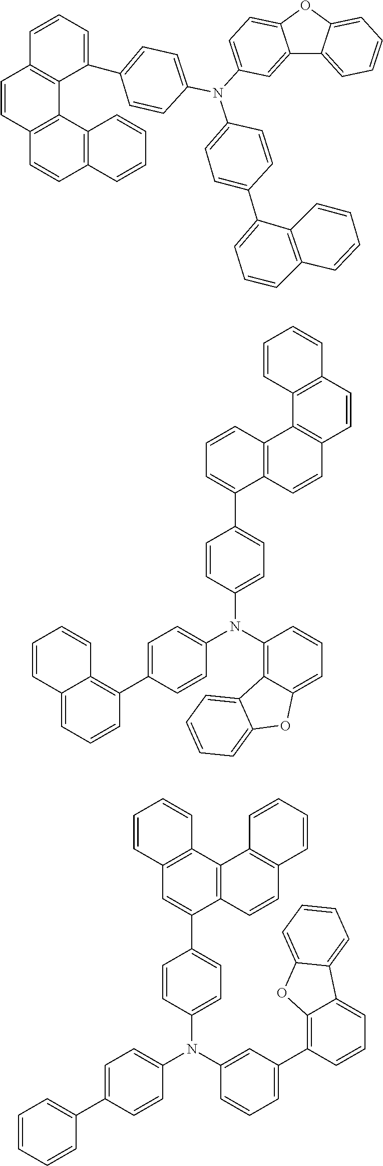

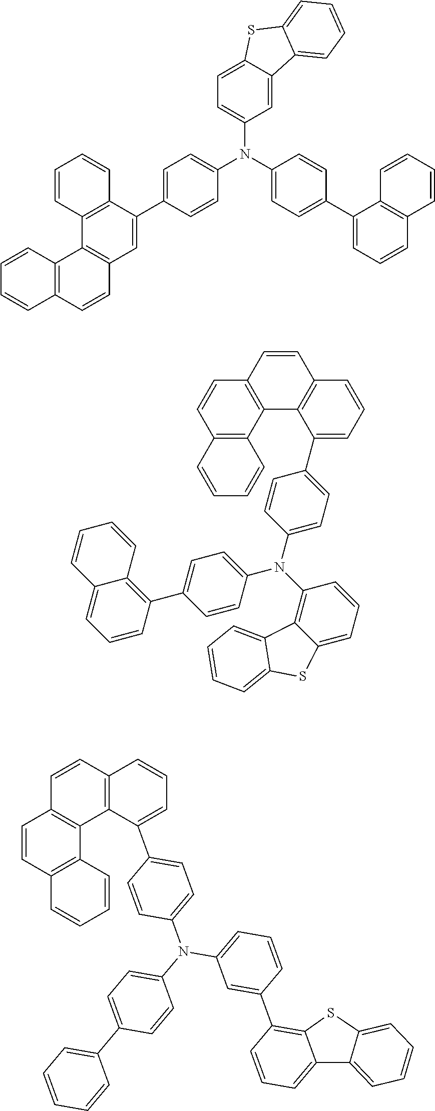 [SMILES: c1ccc2c(-c3ccc(N(c4ccc(-c5cc6ccc7ccccc7c6c6ccccc56)cc4)c4ccc5sc6ccccc6c5c4)cc3)cccc2c1 ; c1ccc2c(-c3ccc(N(c4ccc(-c5cccc6ccc7ccc8ccccc8c7c56)cc4)c4cccc5sc6ccccc6c45)cc3)cccc2c1 ; c1ccc(-c2ccc(N(c3ccc(-c4cccc5ccc6ccc7ccccc7c6c45)cc3)c3cccc(-c4cccc5c4sc4ccccc45)c3)cc2)cc1]

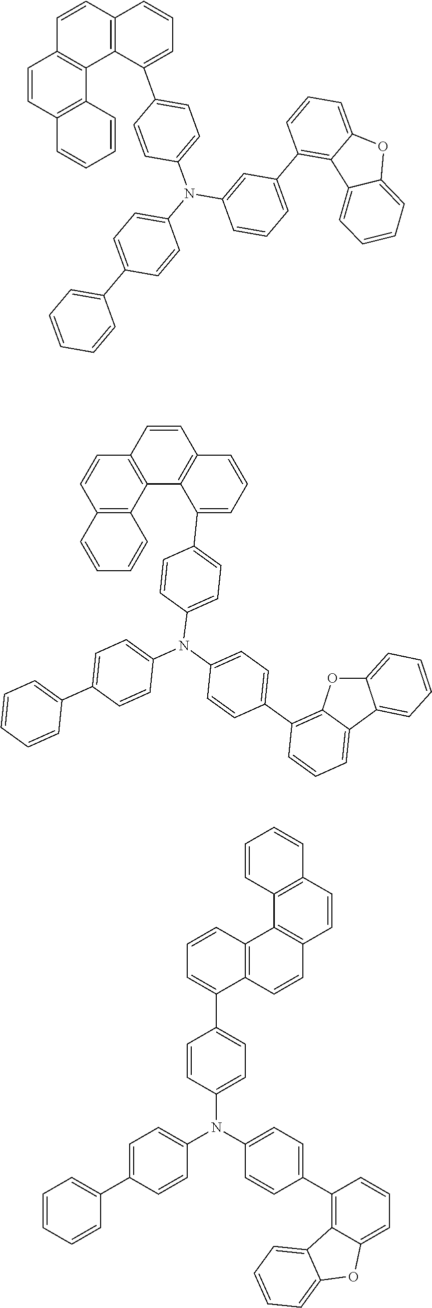 [SMILES: c1ccc(-c2ccc(N(c3ccc(-c4cccc5ccc6ccc7ccccc7c6c45)cc3)c3cccc(-c4cccc5oc6ccccc6c45)c3)cc2)cc1 ; c1ccc(-c2ccc(N(c3ccc(-c4cccc5c4oc4ccccc45)cc3)c3ccc(-c4cccc5ccc6ccc7ccccc7c6c45)cc3)cc2)cc1 ; c1ccc(-c2ccc(N(c3ccc(-c4cccc5c4ccc4ccc6ccccc6c45)cc3)c3ccc(-c4cccc5oc6ccccc6c45)cc3)cc2)cc1]

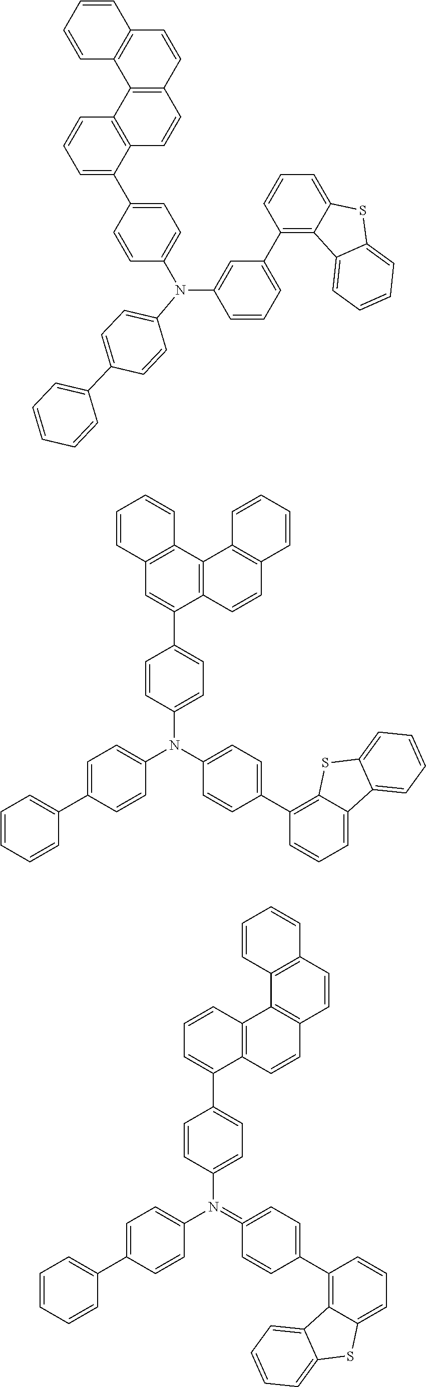

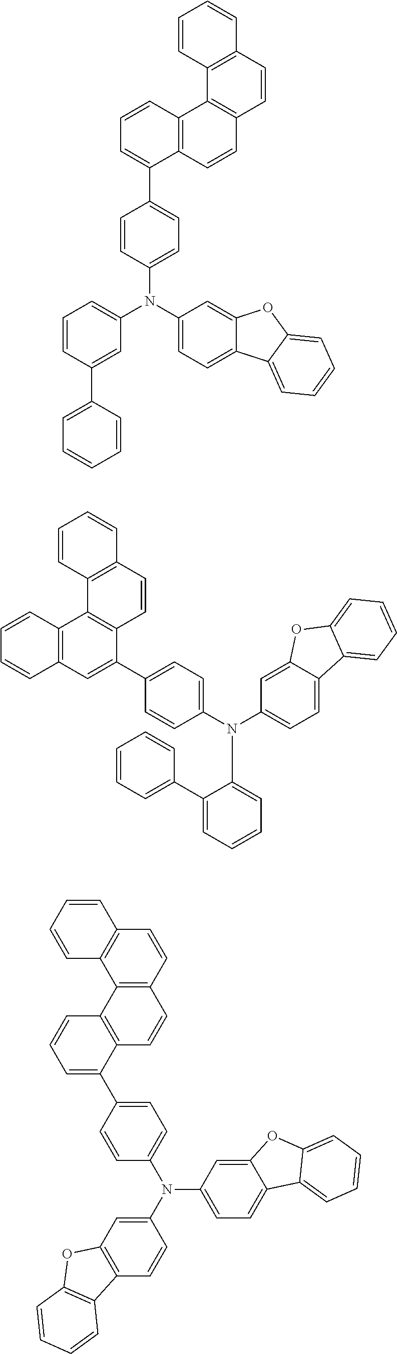

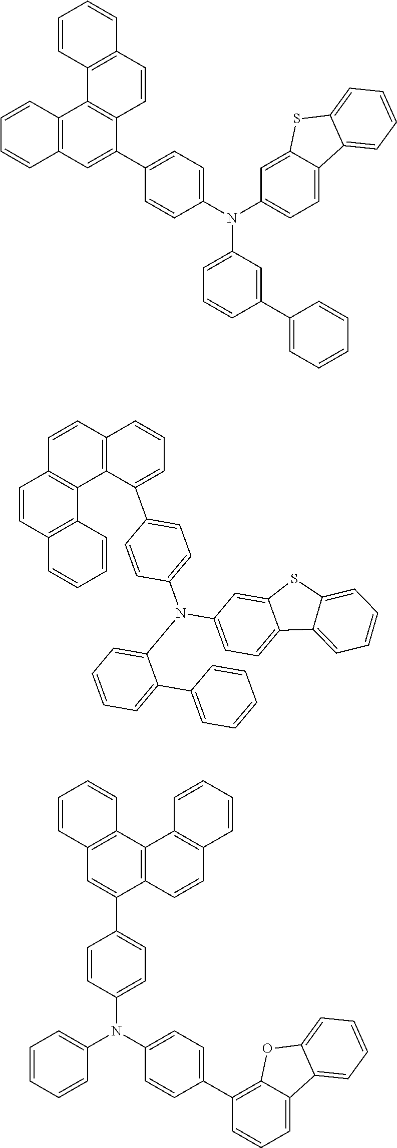 [SMILES: c1ccc(-c2cccc(N(c3ccc(-c4cc5ccccc5c5c4ccc4ccccc45)cc3)c3ccc4c(c3)sc3ccccc34)c2)cc1 ; c1ccc(-c2ccccc2N(c2ccc(-c3cccc4ccc5ccc6ccccc6c5c34)cc2)c2ccc3c(c2)sc2ccccc23)cc1 ; c1ccc(N(c2ccc(-c3cc4ccccc4c4c3ccc3ccccc34)cc2)c2ccc(-c3cccc4c3oc3ccccc34)cc2)cc1]

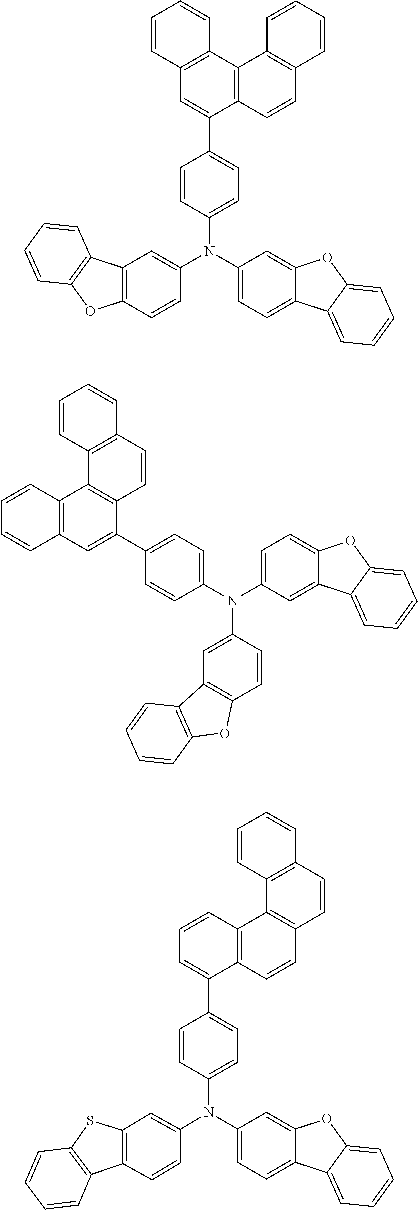 [SMILES: c1ccc2c(c1)ccc1c(-c3ccc(N(c4ccc5c(c4)oc4ccccc45)c4ccc5oc6ccccc6c5c4)cc3)cc3ccccc3c12 ; c1ccc2c(c1)ccc1c(-c3ccc(N(c4ccc5oc6ccccc6c5c4)c4ccc5oc6ccccc6c5c4)cc3)cc3ccccc3c12 ; c1ccc2c(c1)ccc1ccc3c(-c4ccc(N(c5ccc6c(c5)oc5ccccc56)c5ccc6c(c5)sc5ccccc56)cc4)cccc3c12]

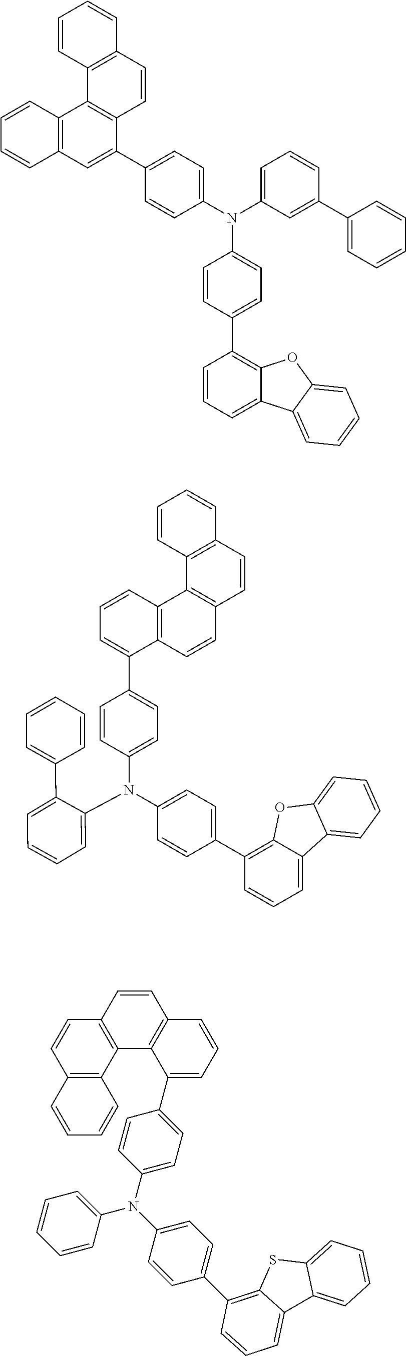 [SMILES: c1ccc(-c2cccc(N(c3ccc(-c4cc5ccccc5c5c4ccc4ccccc45)cc3)c3ccc(-c4cccc5c4oc4ccccc45)cc3)c2)cc1 ; c1ccc(-c2ccccc2N(c2ccc(-c3cccc4c3ccc3ccc5ccccc5c34)cc2)c2ccc(-c3cccc4c3oc3ccccc34)cc2)cc1 ; c1ccc(N(c2ccc(-c3cccc4c3sc3ccccc34)cc2)c2ccc(-c3cccc4ccc5ccc6ccccc6c5c34)cc2)cc1]

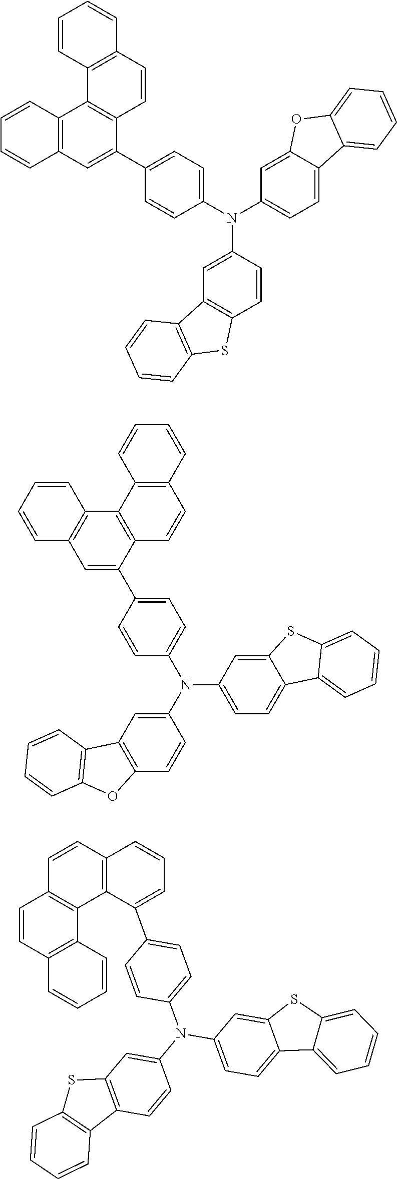

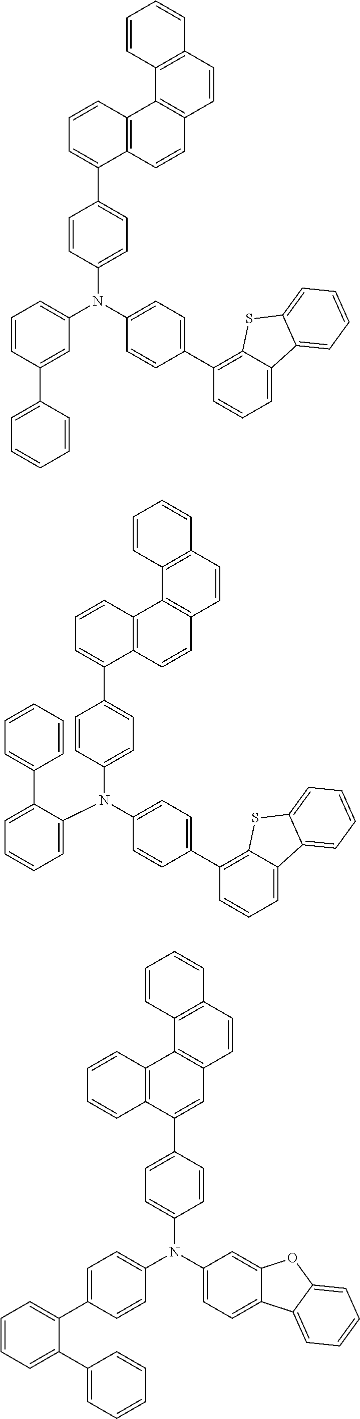 [SMILES: c1ccc(-c2cccc(N(c3ccc(-c4cccc5c4ccc4ccc6ccccc6c45)cc3)c3ccc(-c4cccc5c4sc4ccccc45)cc3)c2)cc1 ; c1ccc(-c2ccccc2N(c2ccc(-c3cccc4c3ccc3ccc5ccccc5c34)cc2)c2ccc(-c3cccc4c3sc3ccccc34)cc2)cc1 ; c1ccc(-c2ccccc2-c2ccc(N(c3ccc(-c4cc5ccc6ccccc6c5c5ccccc45)cc3)c3ccc4c(c3)oc3ccccc34)cc2)cc1]

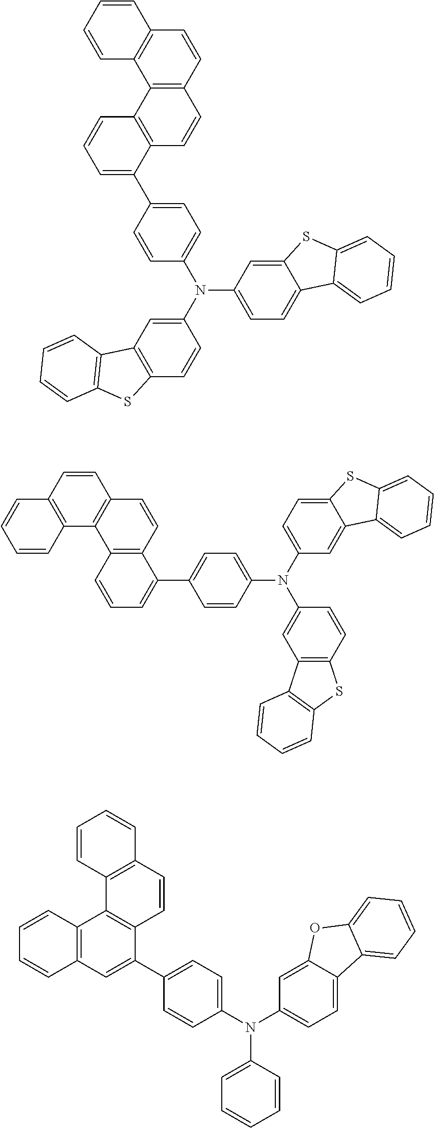 [SMILES: c1ccc2c(c1)ccc1ccc3c(-c4ccc(N(c5ccc6c(c5)sc5ccccc56)c5ccc6sc7ccccc7c6c5)cc4)cccc3c12 ; c1ccc2c(c1)ccc1ccc3c(-c4ccc(N(c5ccc6sc7ccccc7c6c5)c5ccc6sc7ccccc7c6c5)cc4)cccc3c12 ; c1ccc(N(c2ccc(-c3cc4ccccc4c4c3ccc3ccccc34)cc2)c2ccc3c(c2)oc2ccccc23)cc1]

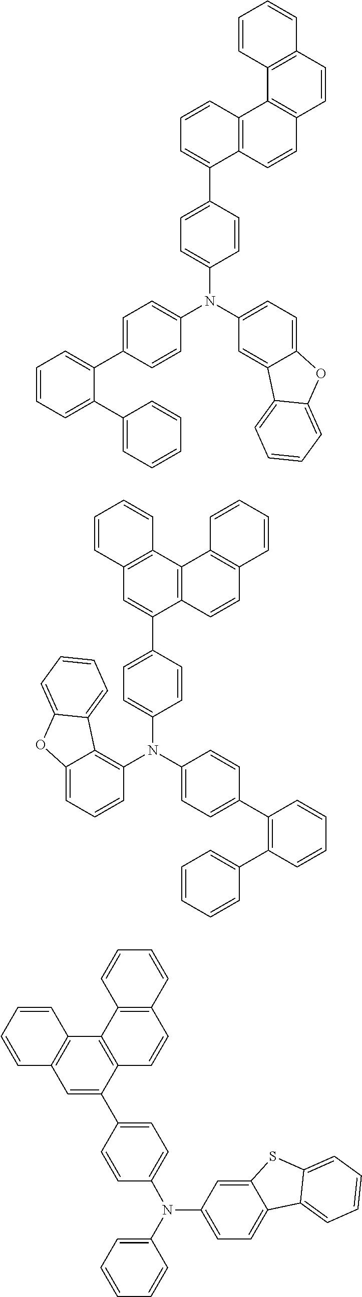

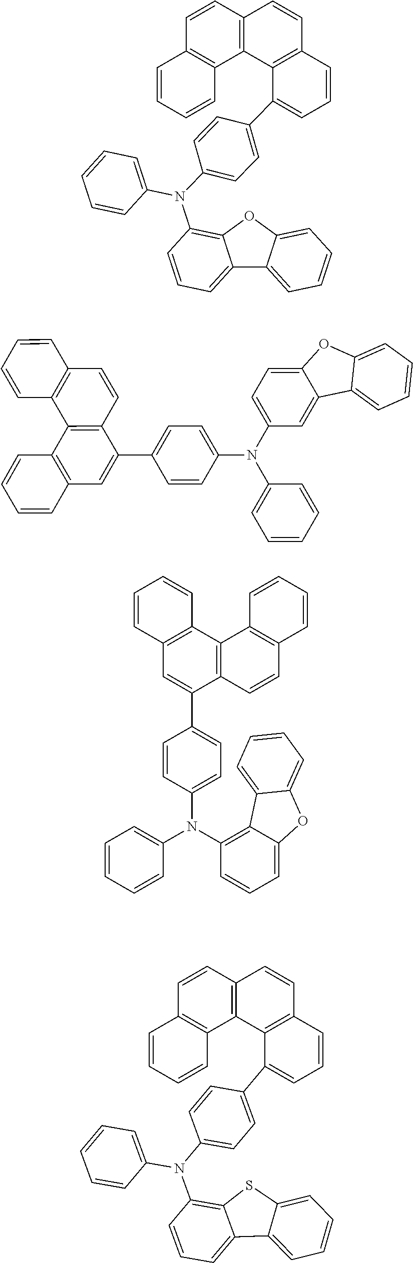

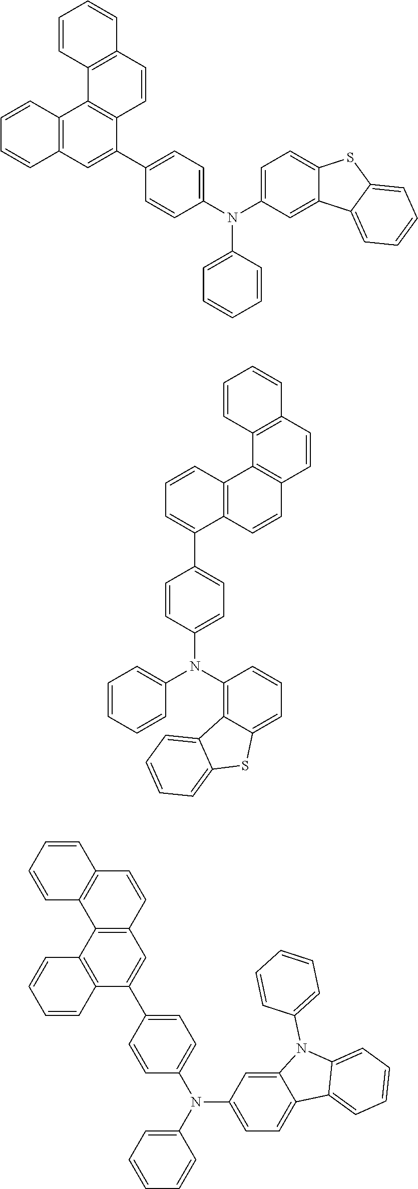 [SMILES: c1ccc(N(c2ccc(-c3cc4ccccc4c4c3ccc3ccccc34)cc2)c2ccc3sc4ccccc4c3c2)cc1 ; c1ccc(N(c2ccc(-c3cccc4c3ccc3ccc5ccccc5c34)cc2)c2cccc3sc4ccccc4c23)cc1 ; c1ccc(N(c2ccc(-c3cc4ccc5ccccc5c4c4ccccc34)cc2)c2ccc3c4ccccc4n(-c4ccccc4)c3c2)cc1]

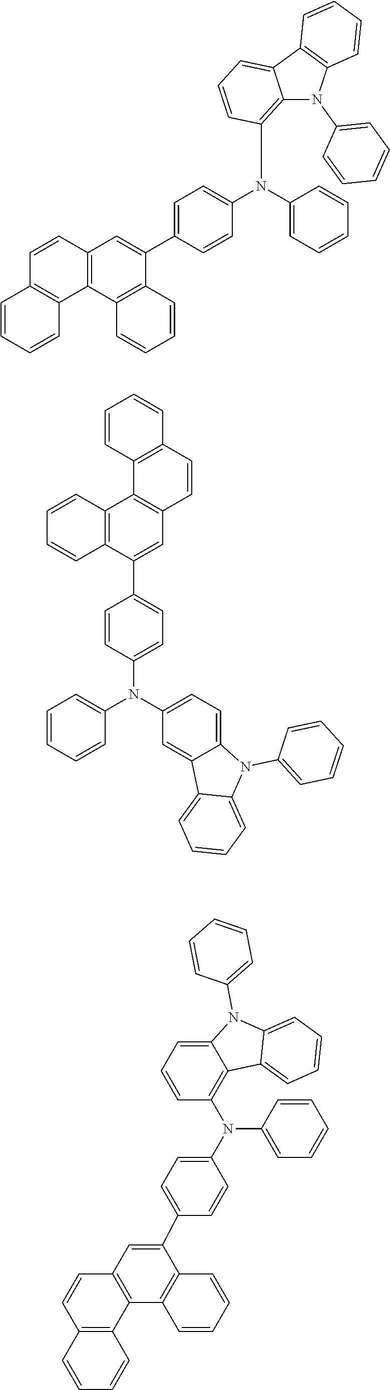

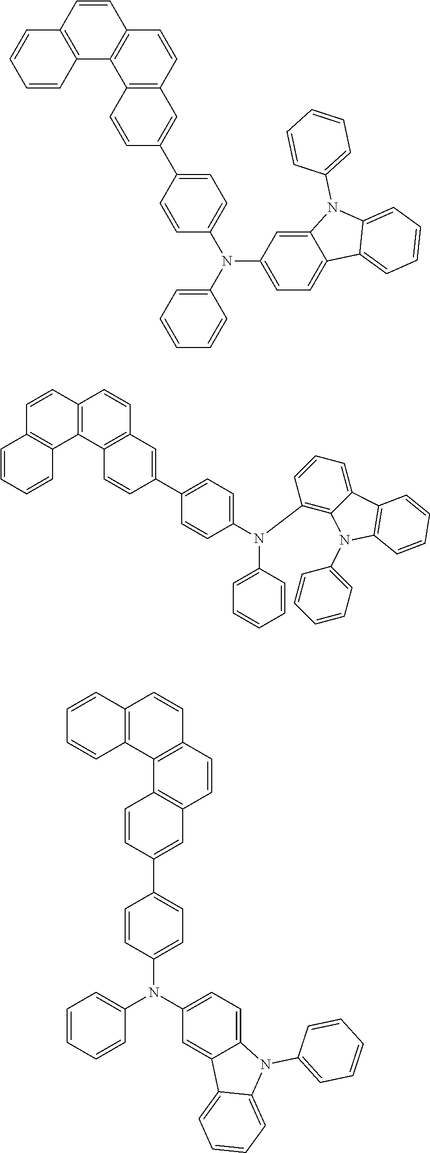 [SMILES: c1ccc(N(c2ccc(-c3ccc4c(ccc5ccc6ccccc6c54)c3)cc2)c2ccc3c4ccccc4n(-c4ccccc4)c3c2)cc1 ; c1ccc(N(c2ccc(-c3ccc4c(ccc5ccc6ccccc6c54)c3)cc2)c2cccc3c4ccccc4n(-c4ccccc4)c23)cc1 ; c1ccc(N(c2ccc(-c3ccc4c(ccc5ccc6ccccc6c54)c3)cc2)c2ccc3c(c2)c2ccccc2n3-c2ccccc2)cc1]

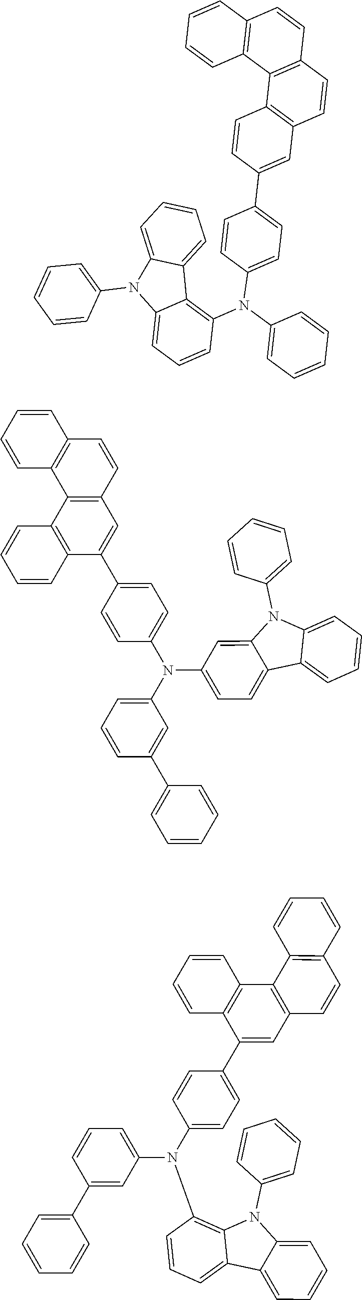 [SMILES: c1ccc(N(c2ccc(-c3ccc4c(ccc5ccc6ccccc6c54)c3)cc2)c2cccc3c2c2ccccc2n3-c2ccccc2)cc1 ; c1ccc(-c2cccc(N(c3ccc(-c4cc5ccc6ccccc6c5c5ccccc45)cc3)c3ccc4c5ccccc5n(-c5ccccc5)c4c3)c2)cc1 ; c1ccc(-c2cccc(N(c3ccc(-c4cc5ccc6ccccc6c5c5ccccc45)cc3)c3cccc4c5ccccc5n(-c5ccccc5)c34)c2)cc1]

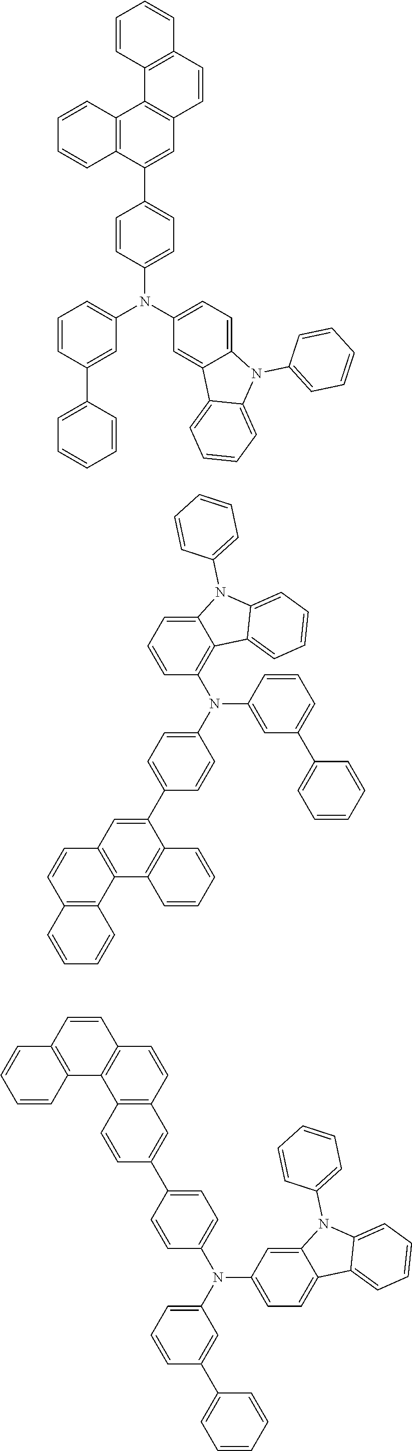

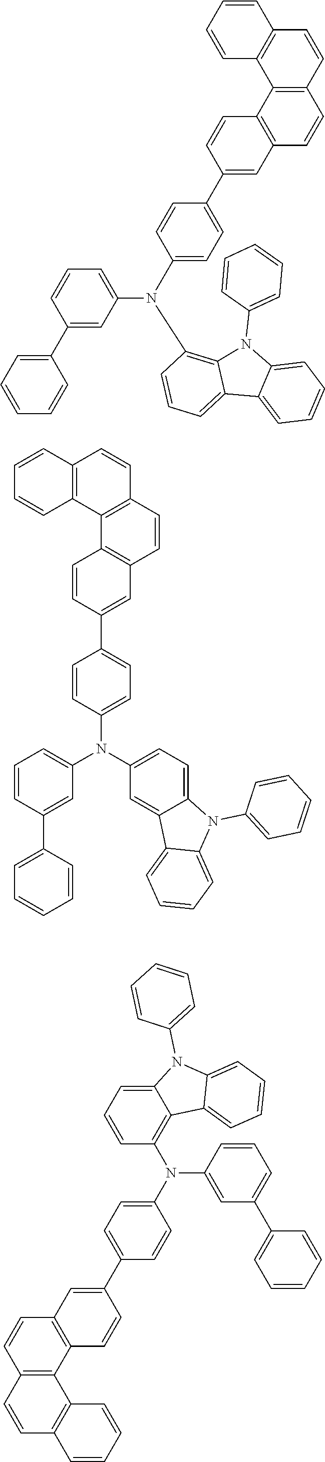

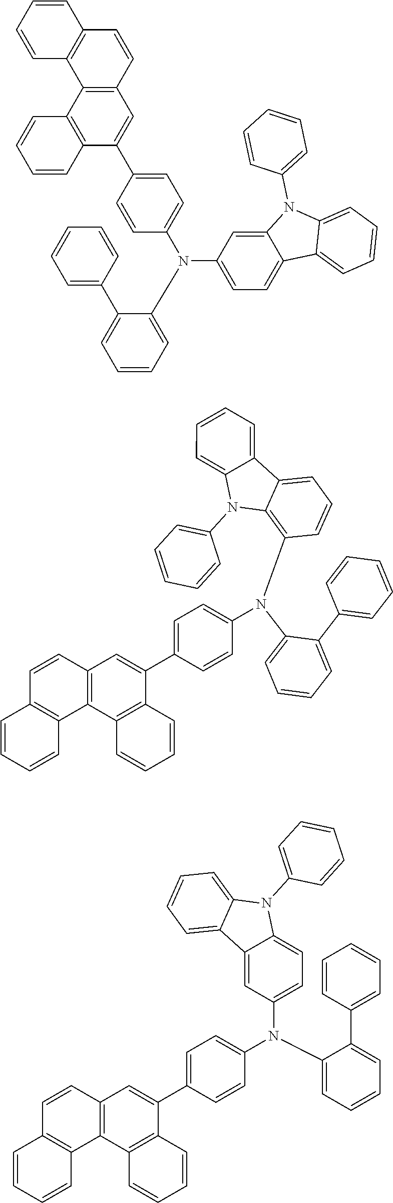 [SMILES: c1ccc(-c2ccccc2N(c2ccc(-c3cc4ccc5ccccc5c4c4ccccc34)cc2)c2ccc3c4ccccc4n(-c4ccccc4)c3c2)cc1 ; c1ccc(-c2ccccc2N(c2ccc(-c3cc4ccc5ccccc5c4c4ccccc34)cc2)c2cccc3c4ccccc4n(-c4ccccc4)c23)cc1 ; c1ccc(-c2ccccc2N(c2ccc(-c3cc4ccc5ccccc5c4c4ccccc34)cc2)c2ccc3c(c2)c2ccccc2n3-c2ccccc2)cc1]

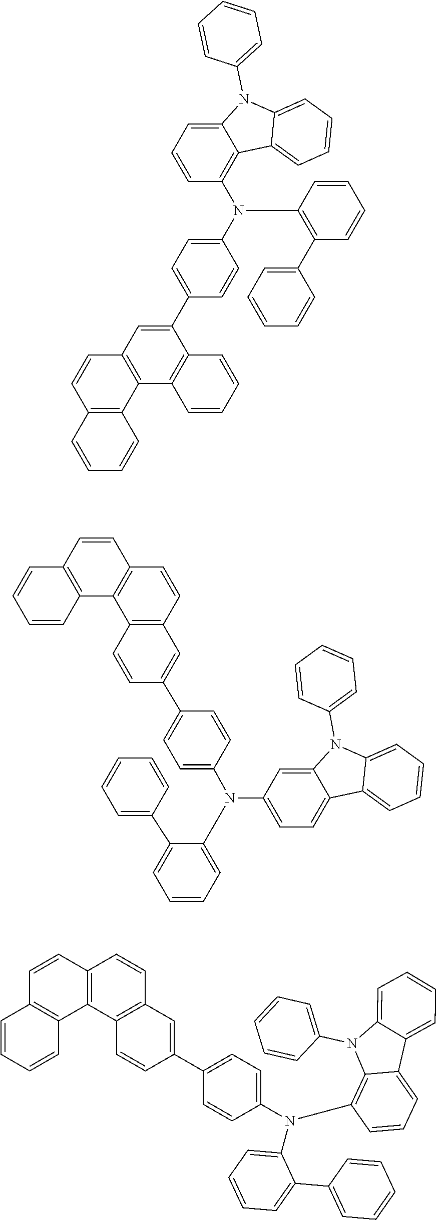 [SMILES: c1ccc(-c2ccccc2N(c2ccc(-c3cc4ccc5ccccc5c4c4ccccc34)cc2)c2cccc3c2c2ccccc2n3-c2ccccc2)cc1 ; c1ccc(-c2ccccc2N(c2ccc(-c3ccc4c(ccc5ccc6ccccc6c54)c3)cc2)c2ccc3c4ccccc4n(-c4ccccc4)c3c2)cc1 ; c1ccc(-c2ccccc2N(c2ccc(-c3ccc4c(ccc5ccc6ccccc6c54)c3)cc2)c2cccc3c4ccccc4n(-c4ccccc4)c23)cc1]

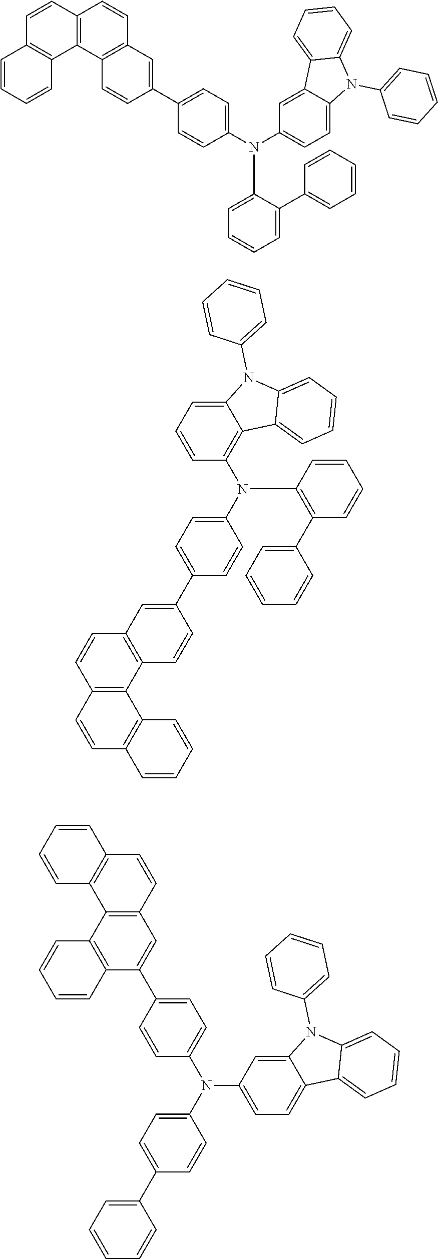

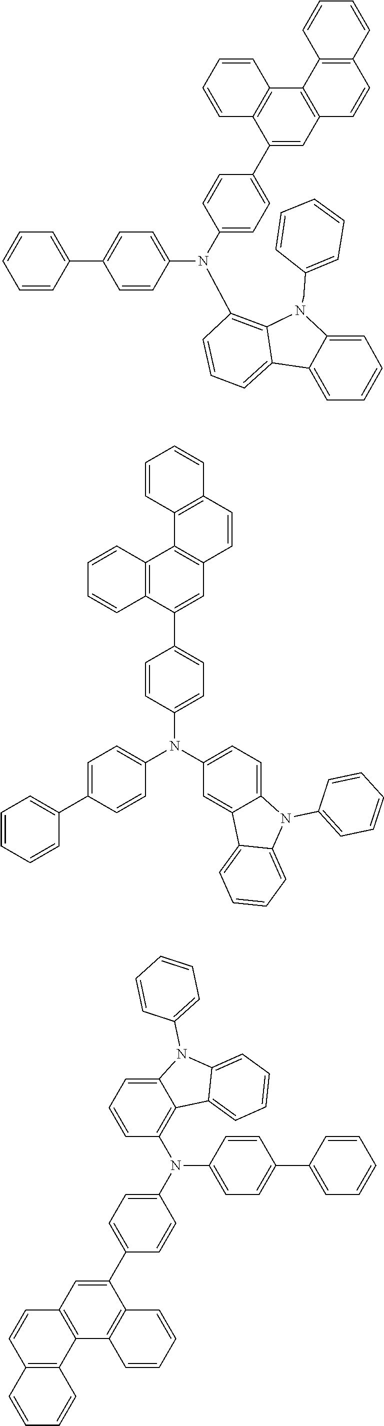 [SMILES: c1ccc(Cn2c3ccccc3c3cccc(N(c4ccc(-c5ccccc5)cc4)c4ccc(-c5cc6ccc7ccccc7c6c6ccccc56)cc4)c32)cc1 ; c1ccc(-c2ccc(N(c3ccc(-c4cc5ccc6ccccc6c5c5ccccc45)cc3)c3ccc4c(c3)c3ccccc3n4-c3ccccc3)cc2)cc1 ; c1ccc(-c2ccc(N(c3ccc(-c4cc5ccc6ccccc6c5c5ccccc45)cc3)c3cccc4c3c3ccccc3n4-c3ccccc3)cc2)cc1]

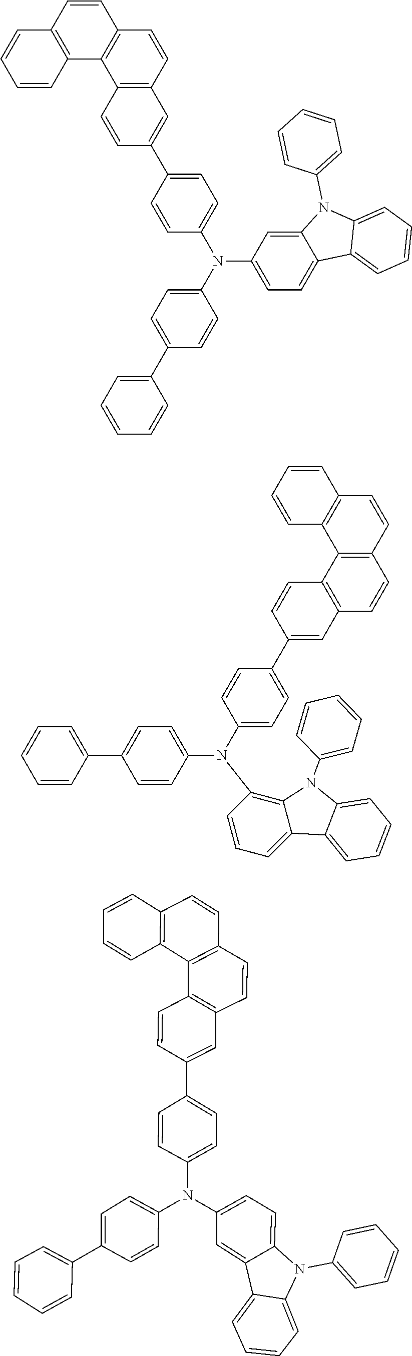 [SMILES: c1ccc(-c2ccc(N(c3ccc(-c4ccc5c(ccc6ccc7ccccc7c65)c4)cc3)c3ccc4c5ccccc5n(-c5ccccc5)c4c3)cc2)cc1 ; c1ccc(-c2ccc(N(c3ccc(-c4ccc5c(ccc6ccc7ccccc7c65)c4)cc3)c3cccc4c5ccccc5n(-c5ccccc5)c34)cc2)cc1 ; c1ccc(-c2ccc(N(c3ccc(-c4ccc5c(ccc6ccc7ccccc7c65)c4)cc3)c3ccc4c(c3)c3ccccc3n4-c3ccccc3)cc2)cc1]

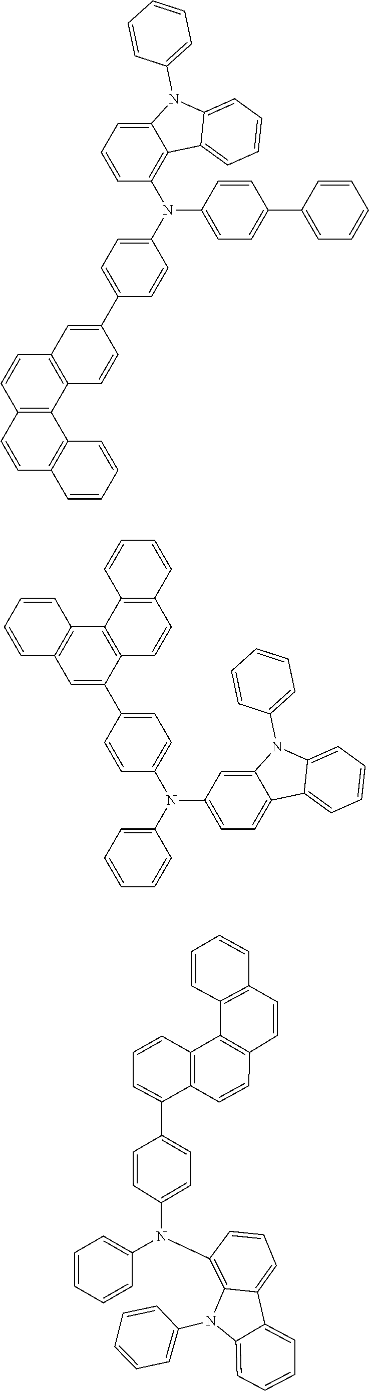

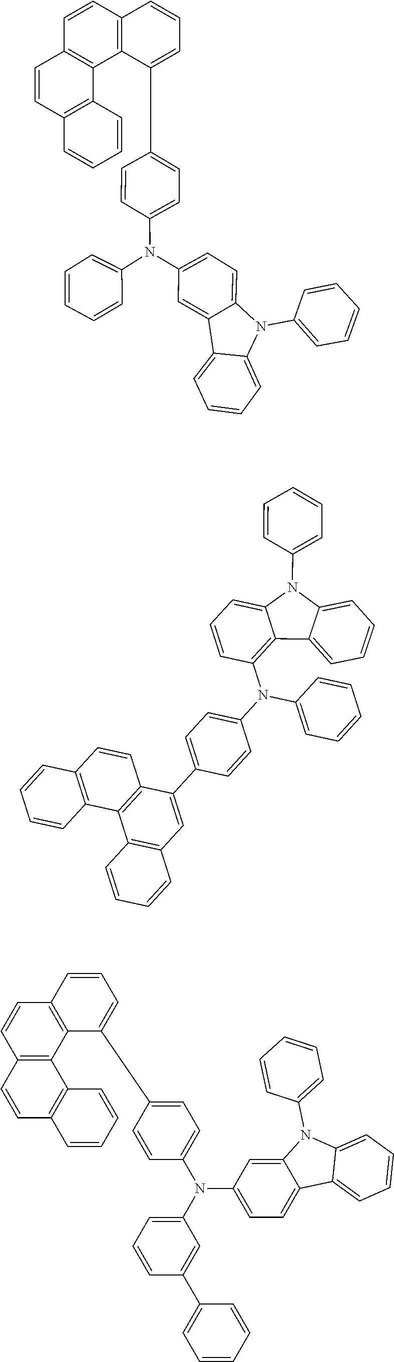 [SMILES: c1ccc(N(c2ccc(-c3cccc4ccc5ccc6ccccc6c5c34)cc2)c2ccc3c(c2)c2ccccc2n3-c2ccccc2)cc1 ; c1ccc(N(c2ccc(-c3cc4ccccc4c4c3ccc3ccccc34)cc2)c2cccc3c2c2ccccc2n3-c2ccccc2)cc1 ; c1ccc(-c2cccc(N(c3ccc(-c4cccc5ccc6ccc7ccccc7c6c45)cc3)c3ccc4c5ccccc5n(-c5ccccc5)c4c3)c2)cc1]

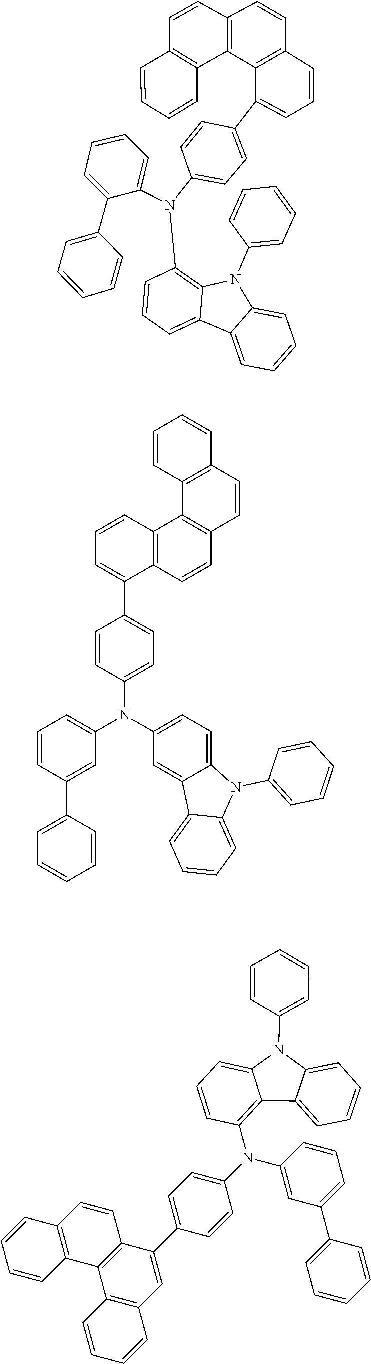 [SMILES: c1ccc(-c2ccccc2N(c2ccc(-c3cccc4ccc5ccc6ccccc6c5c34)cc2)c2cccc3c4ccccc4n(-c4ccccc4)c23)cc1 ; c1ccc(-c2cccc(N(c3ccc(-c4cccc5c4ccc4ccc6ccccc6c45)cc3)c3ccc4c(c3)c3ccccc3n4-c3ccccc3)c2)cc1 ; c1ccc(-c2cccc(N(c3ccc(-c4cc5ccccc5c5c4ccc4ccccc45)cc3)c3cccc4c3c3ccccc3n4-c3ccccc3)c2)cc1]

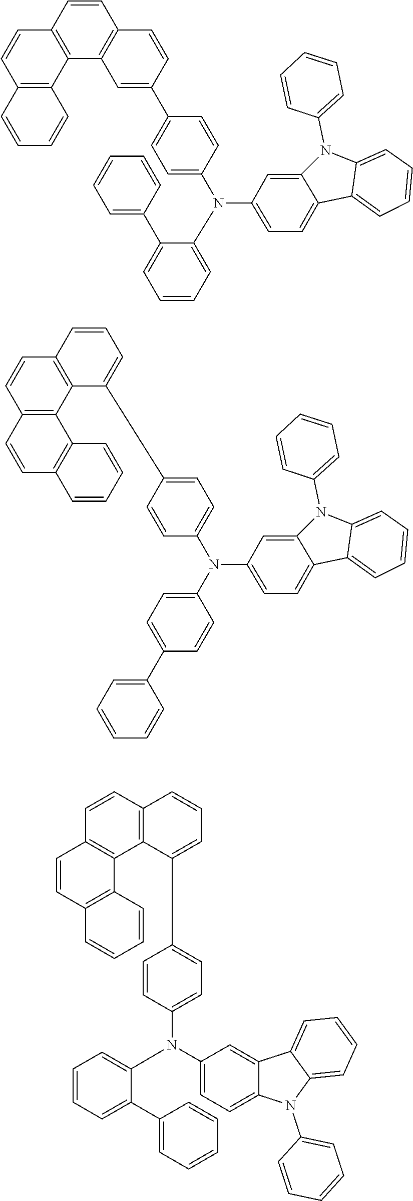 [SMILES: c1ccc(-c2ccccc2N(c2ccc(-c3ccc4ccc5ccc6ccccc6c5c4c3)cc2)c2ccc3c4ccccc4n(-c4ccccc4)c3c2)cc1 ; c1ccc(-c2ccc(N(c3ccc(-c4cccc5ccc6ccc7ccccc7c6c45)cc3)c3ccc4c5ccccc5n(-c5ccccc5)c4c3)cc2)cc1 ; c1ccc(-c2ccccc2N(c2ccc(-c3cccc4ccc5ccc6ccccc6c5c34)cc2)c2ccc3c(c2)c2ccccc2n3-c2ccccc2)cc1]

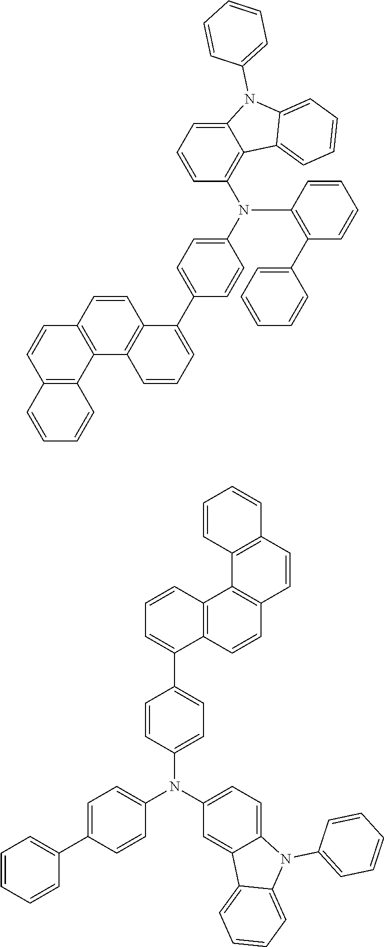 [SMILES: c1ccc(-c2ccccc2N(c2ccc(-c3cccc4c3ccc3ccc5ccccc5c34)cc2)c2cccc3c2c2ccccc2n3-c2ccccc2)cc1 ; c1ccc(-c2ccc(N(c3ccc(-c4cccc5c4ccc4ccc6ccccc6c45)cc3)c3ccc4c(c3)c3ccccc3n4-c3ccccc3)cc2)cc1]

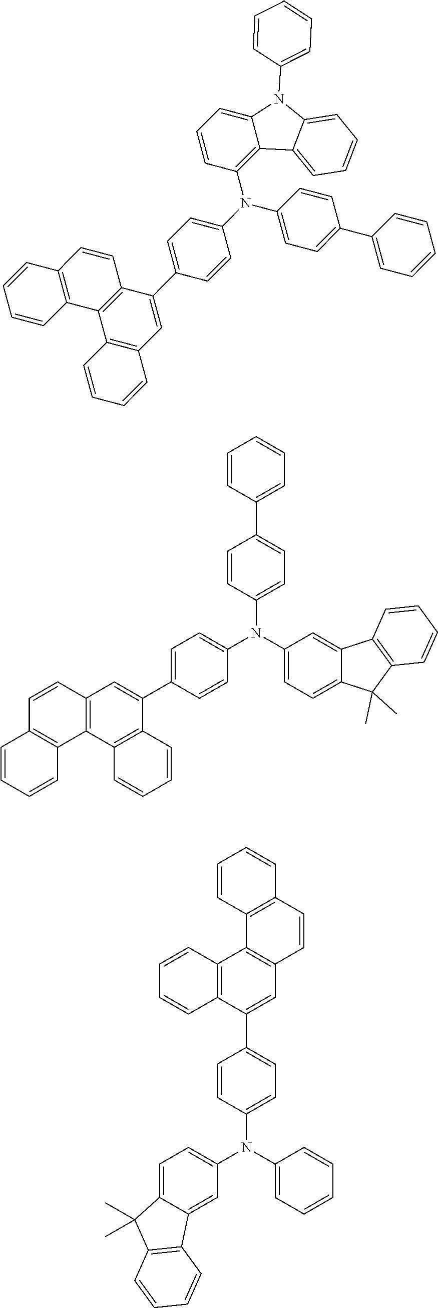 [SMILES: c1ccc(-c2ccc(N(c3ccc(-c4cc5ccccc5c5c4ccc4ccccc45)cc3)c3cccc4c3c3ccccc3n4-c3ccccc3)cc2)cc1 ; CC1(C)c2ccccc2-c2cc(N(c3ccc(-c4ccccc4)cc3)c3ccc(-c4cc5ccc6ccccc6c5c5ccccc45)cc3)ccc21 ; CC1(C)c2ccccc2-c2cc(N(c3ccccc3)c3ccc(-c4cc5ccc6ccccc6c5c5ccccc45)cc3)ccc21]

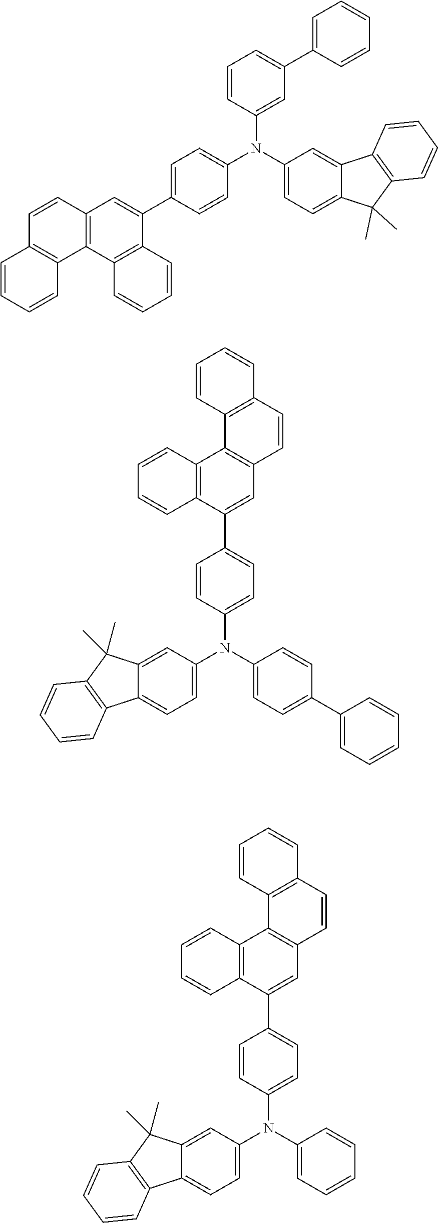 [SMILES: CC1(C)c2ccccc2-c2cc(N(c3ccc(-c4cc5ccc6ccccc6c5c5ccccc45)cc3)c3cccc(-c4ccccc4)c3)ccc21 ; CC1(C)c2ccccc2-c2ccc(N(c3ccc(-c4ccccc4)cc3)c3ccc(-c4cc5ccc6ccccc6c5c5ccccc45)cc3)cc21 ; CC1(C)c2ccccc2-c2ccc(N(c3ccccc3)c3ccc(-c4cc5ccc6ccccc6c5c5ccccc45)cc3)cc21]

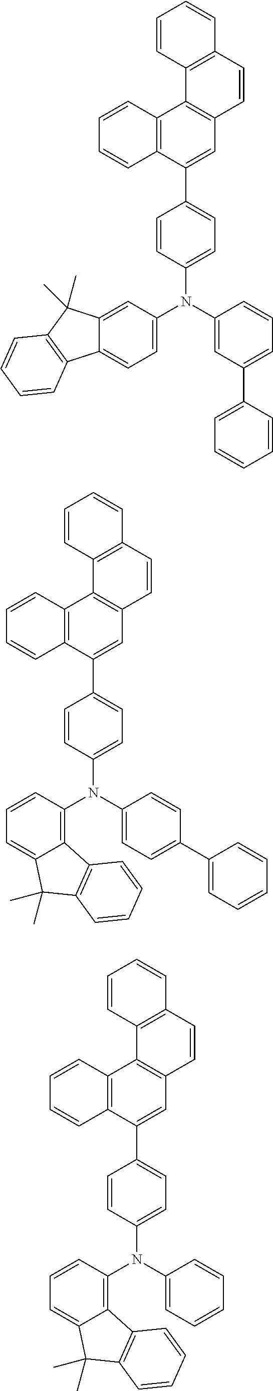

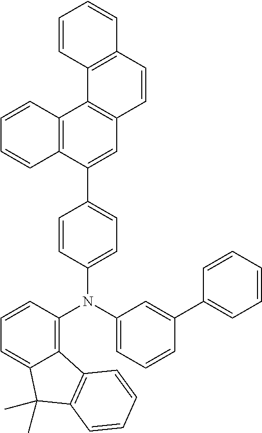 [SMILES: CC1(C)c2ccccc2-c2c(N(c3ccc(-c4cc5ccc6ccccc6c5c5ccccc45)cc3)c3cccc(-c4ccccc4)c3)cccc21]

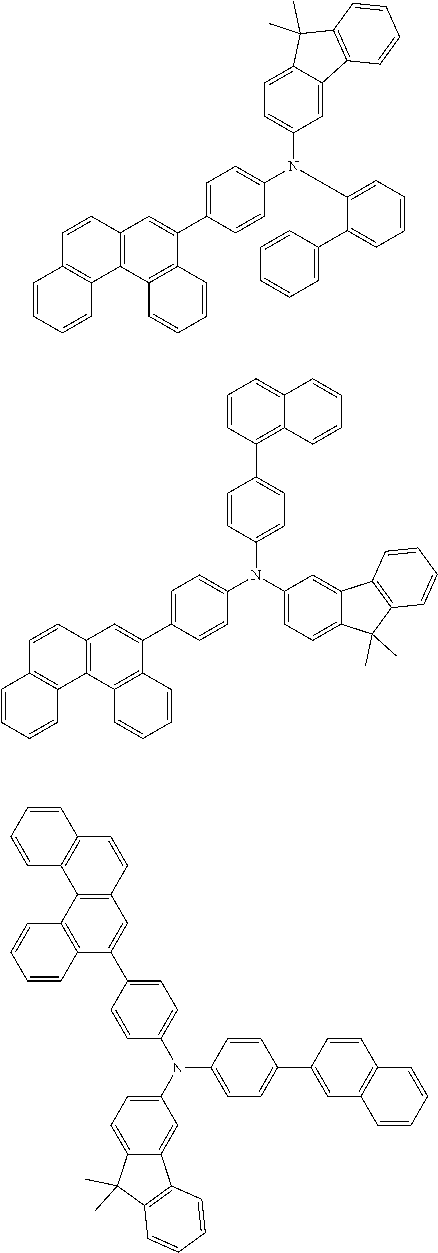 [SMILES: CC1(C)c2ccccc2-c2cc(N(c3ccc(-c4cc5ccc6ccccc6c5c5ccccc45)cc3)c3ccccc3-c3ccccc3)ccc21 ; CC1(C)c2ccccc2-c2cc(N(c3ccc(-c4cccc5ccccc45)cc3)c3ccc(-c4cc5ccc6ccccc6c5c5ccccc45)cc3)ccc21 ; CC1(C)c2ccccc2-c2cc(N(c3ccc(-c4ccc5ccccc5c4)cc3)c3ccc(-c4cc5ccc6ccccc6c5c5ccccc45)cc3)ccc21]

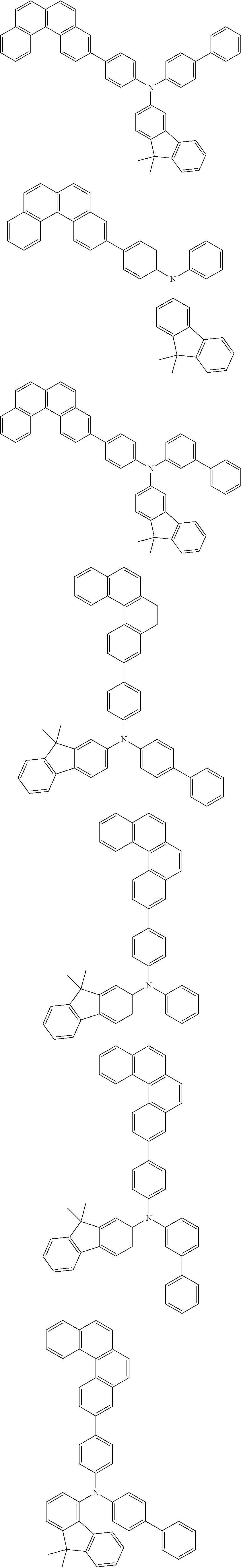

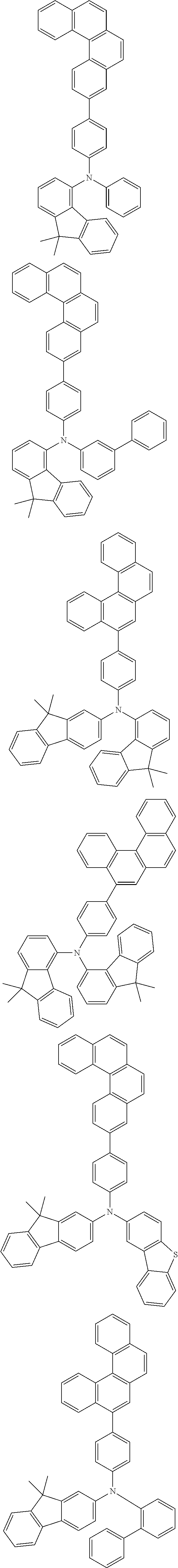

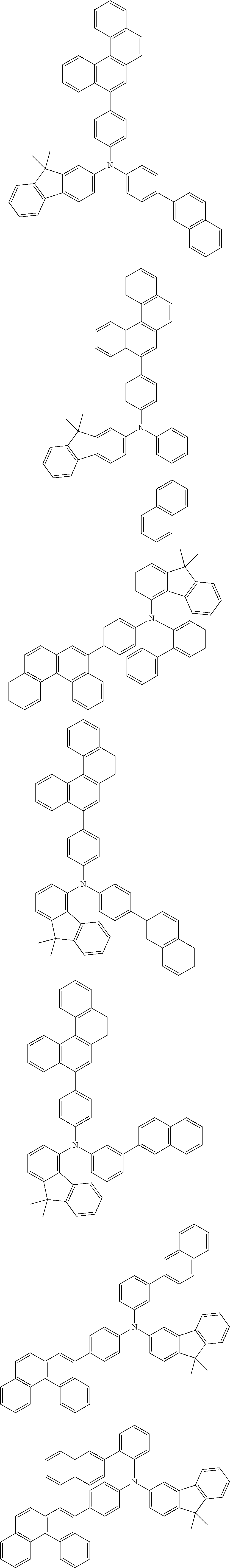

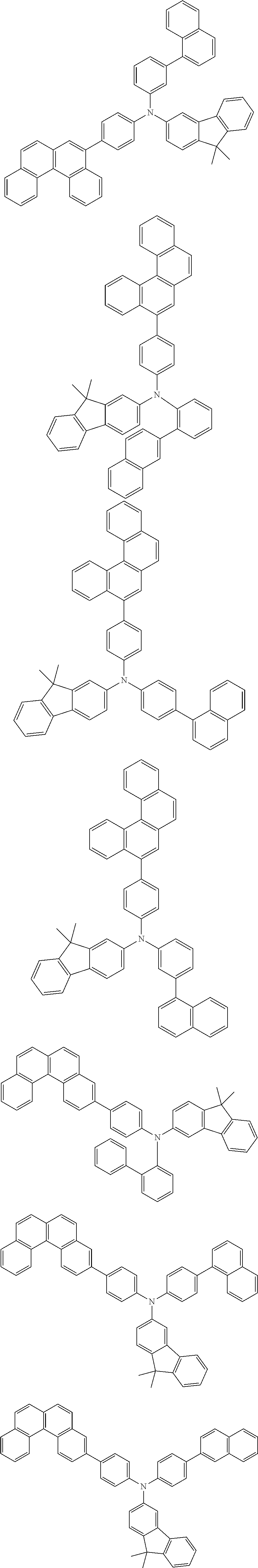

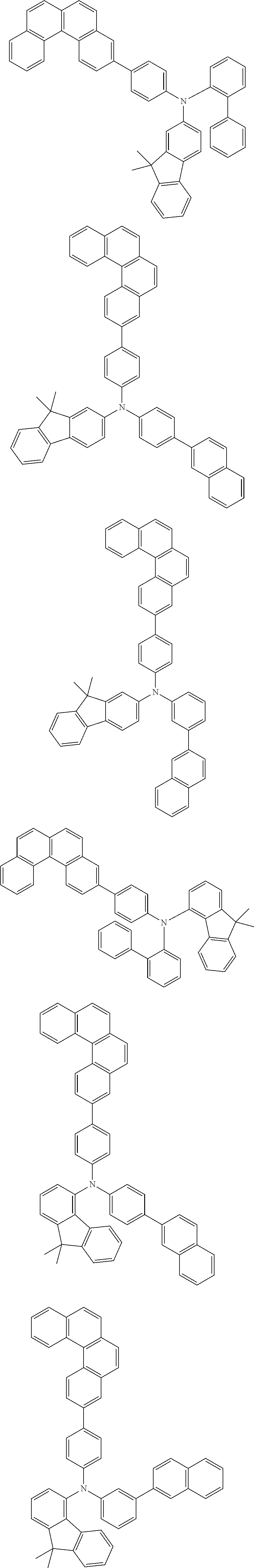

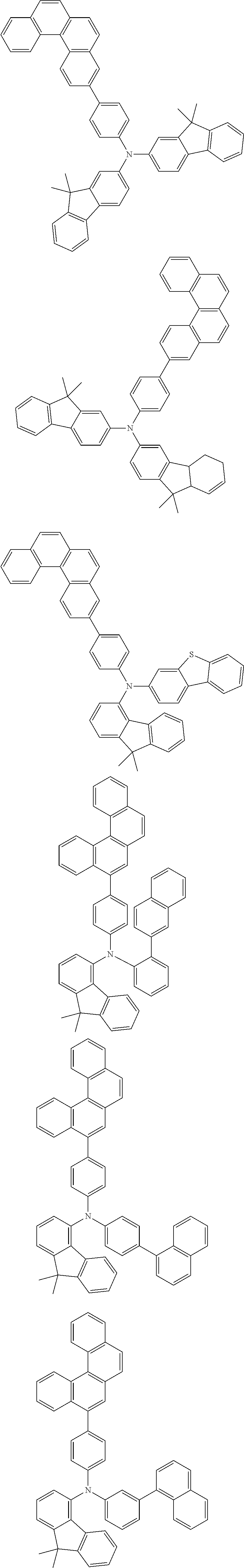

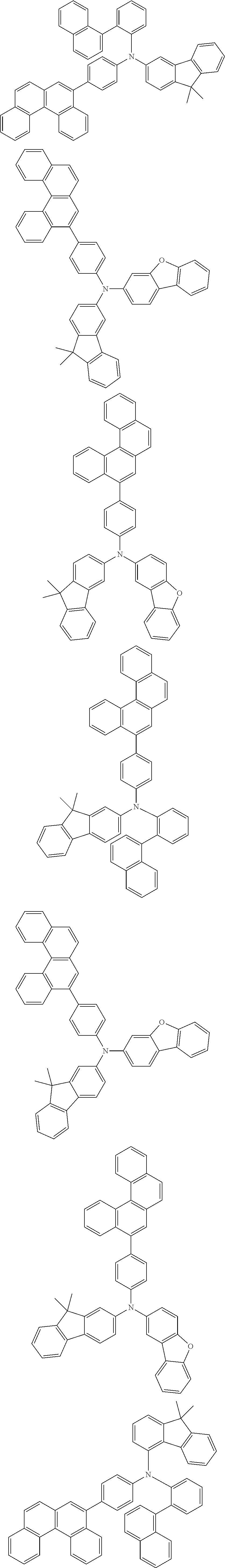

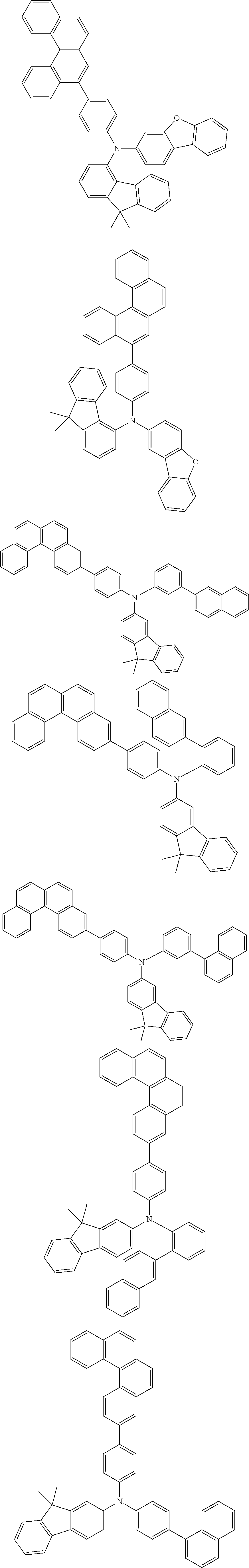

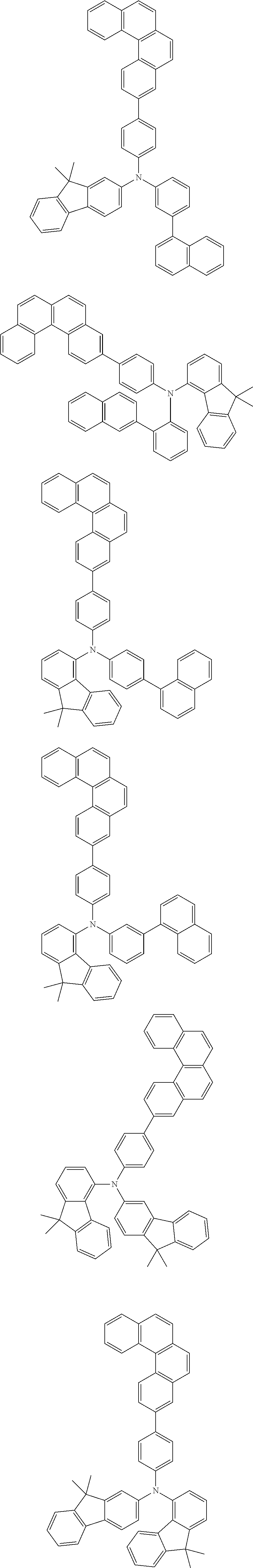

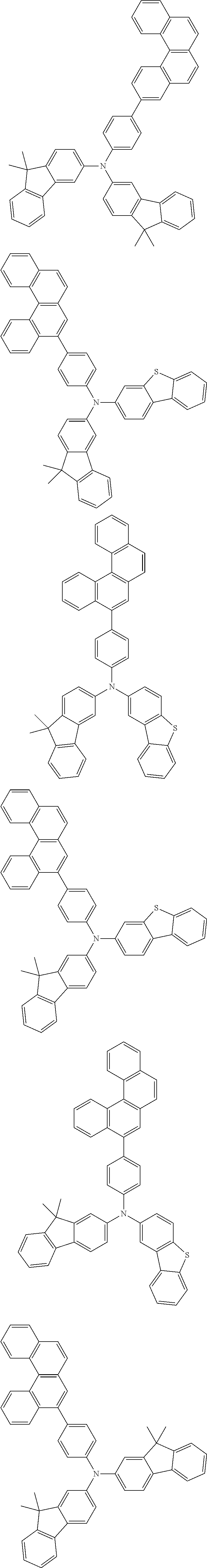

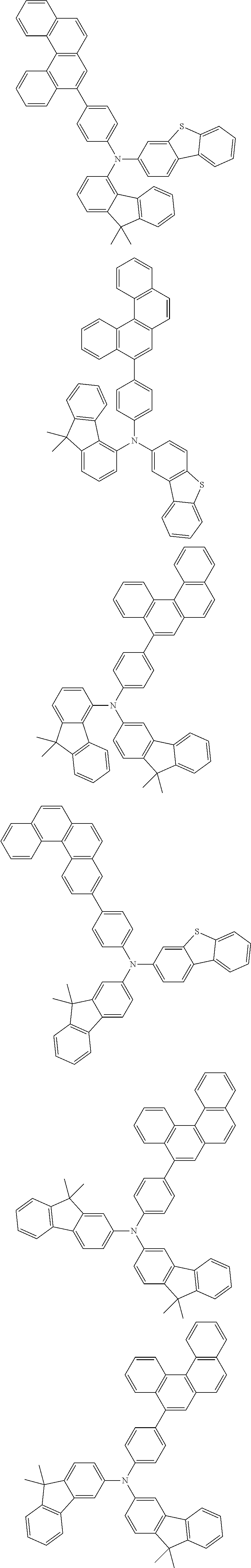

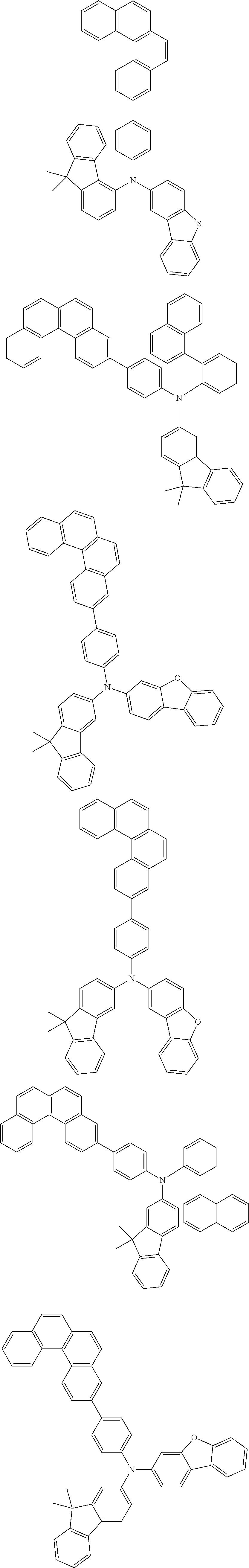

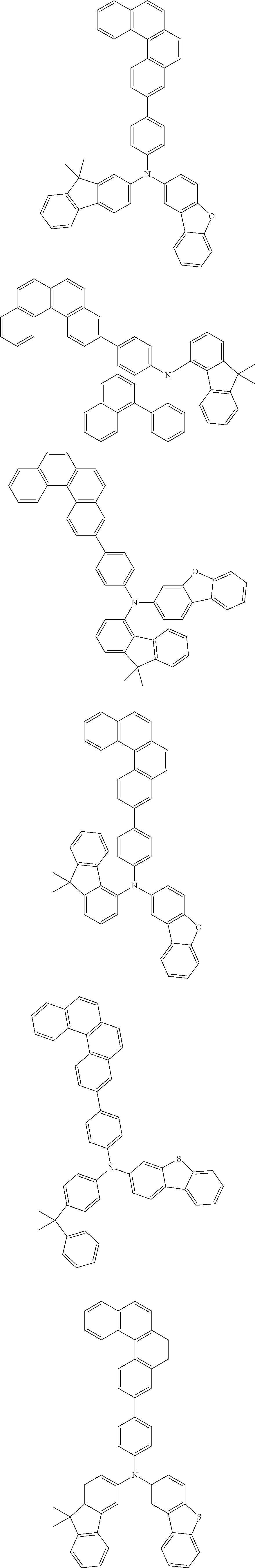

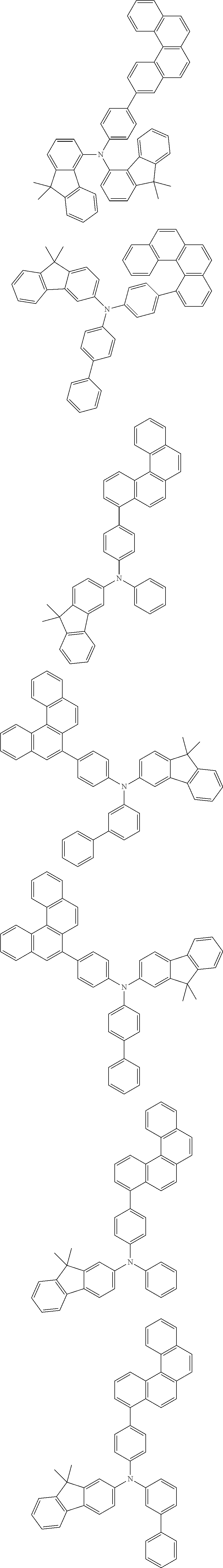

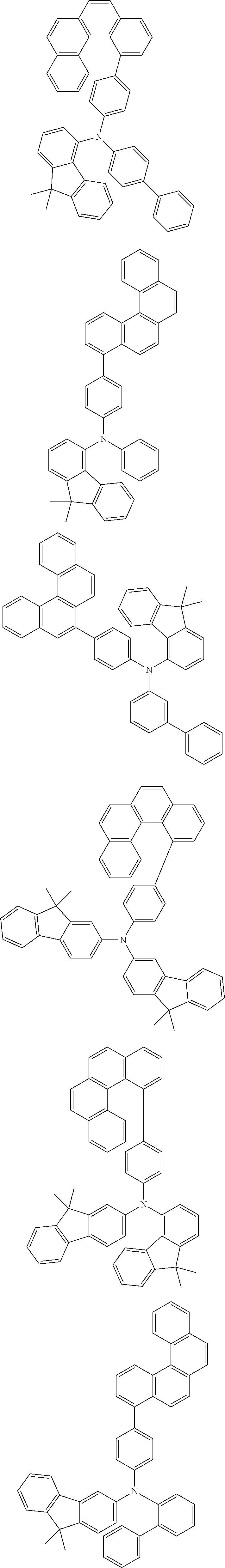

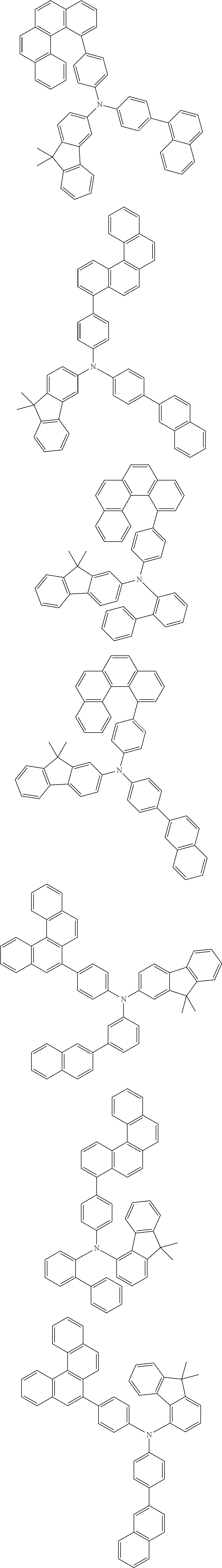

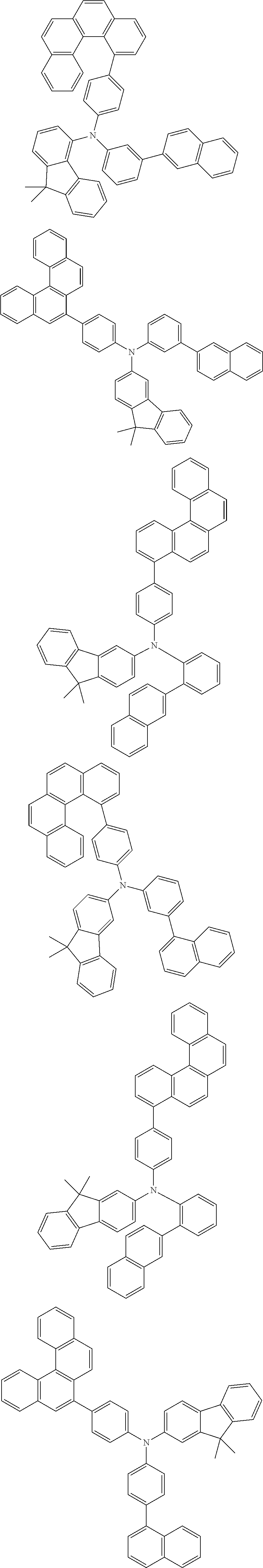

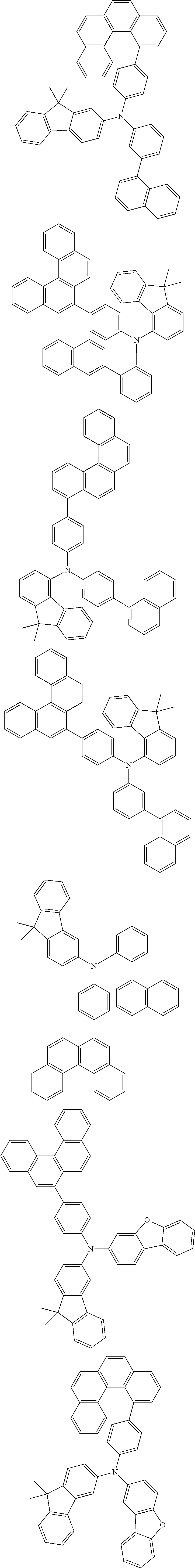

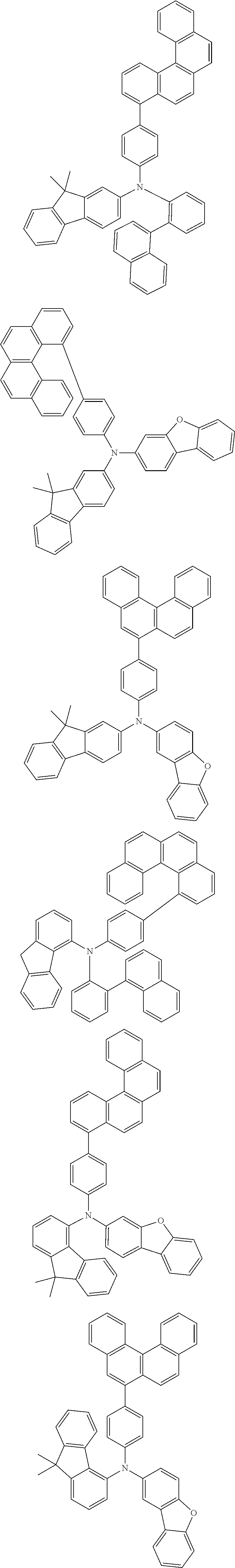

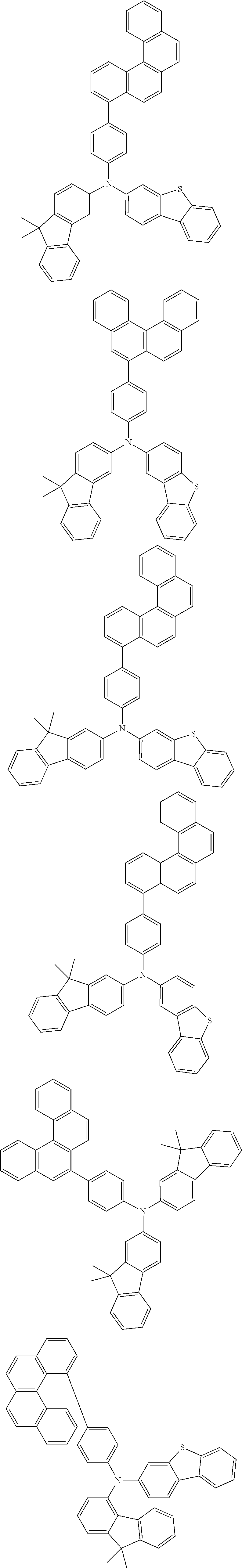

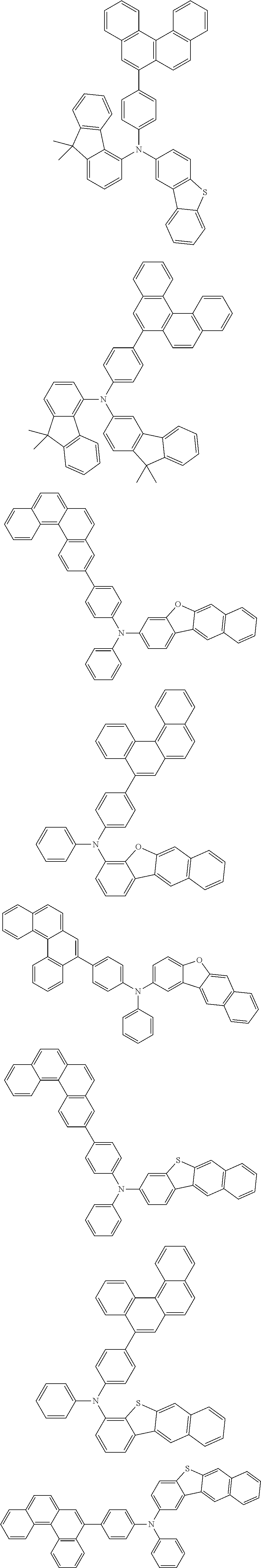

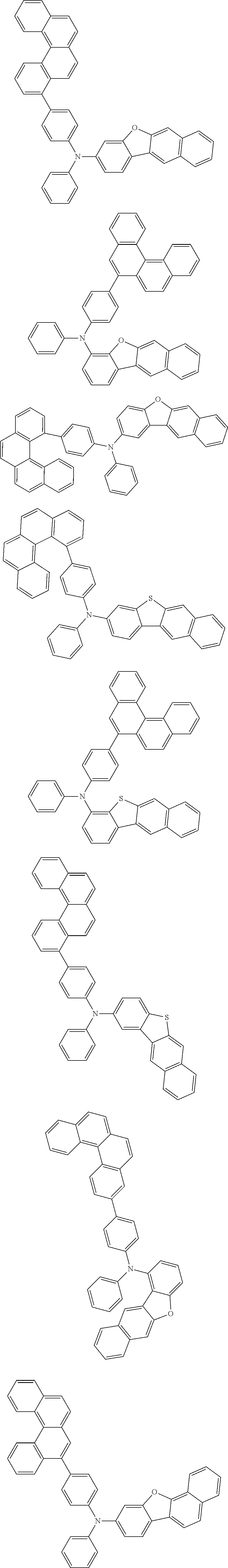

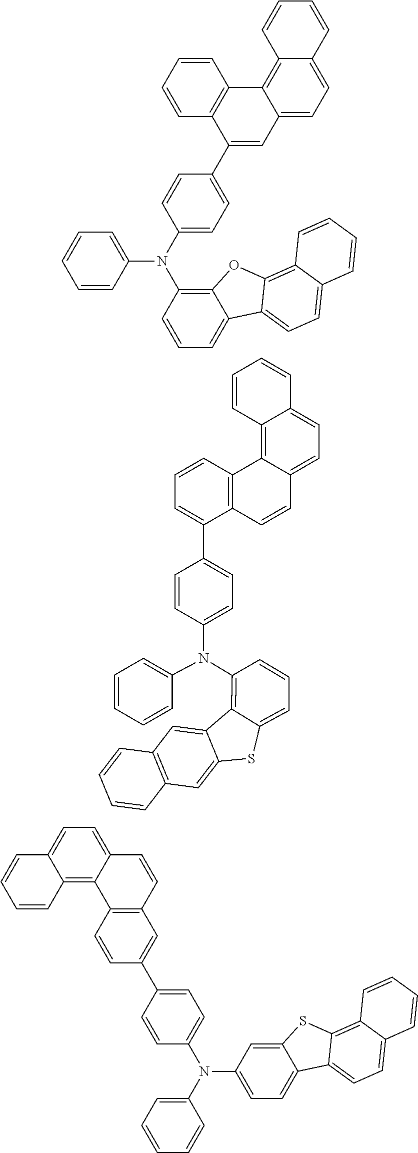

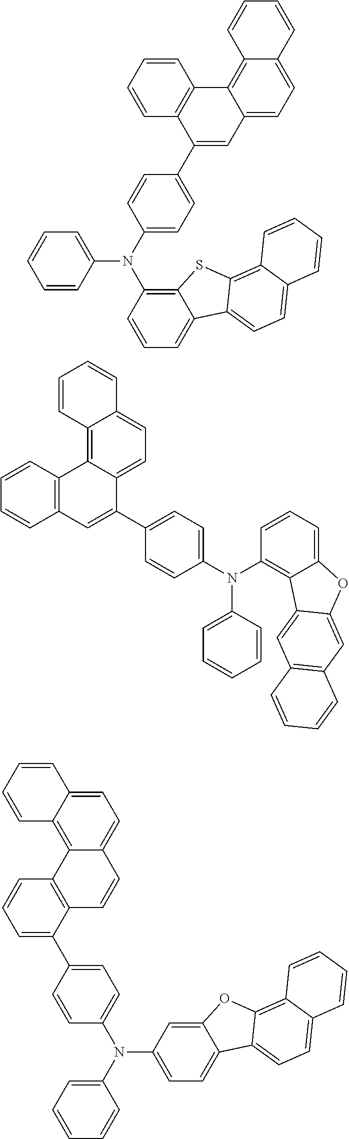

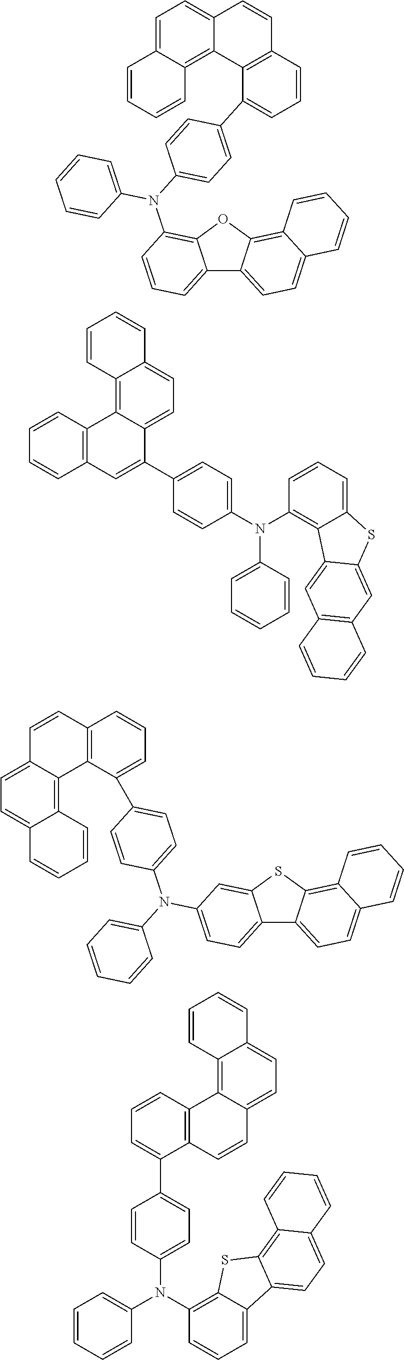

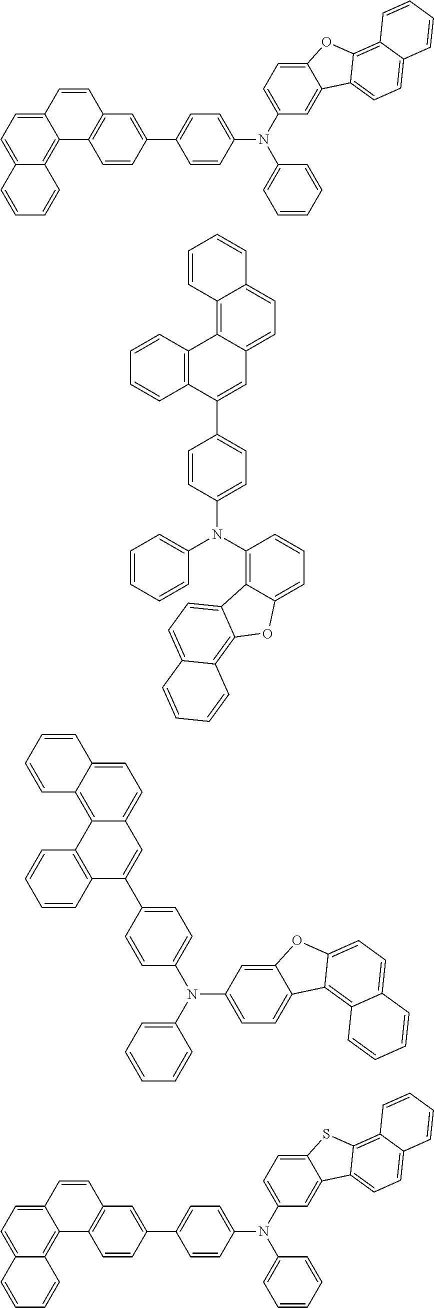 [SMILES: c1ccc(N(c2ccc(-c3ccc4c(ccc5ccc6ccccc6c54)c3)cc2)c2ccc3oc4c5ccccc5ccc4c3c2)cc1 ; c1ccc(N(c2ccc(-c3cc4ccc5ccccc5c4c4ccccc34)cc2)c2cccc3oc4c5ccccc5ccc4c23)cc1 ; c1ccc(N(c2ccc(-c3cc4ccc5ccccc5c4c4ccccc34)cc2)c2ccc3c(c2)oc2ccc4ccccc4c23)cc1 ; c1ccc(N(c2ccc(-c3ccc4c(ccc5ccc6ccccc6c54)c3)cc2)c2ccc3sc4c5ccccc5ccc4c3c2)cc1]

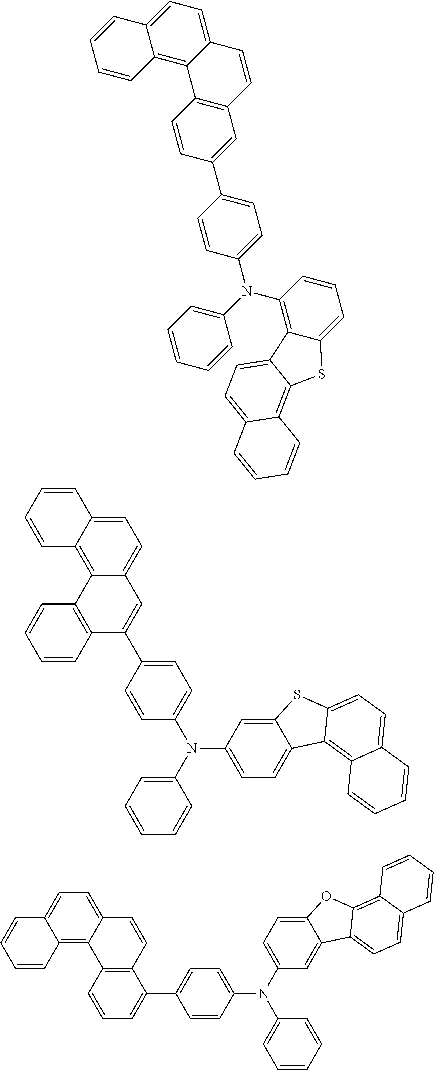 [SMILES: c1ccc(N(c2ccc(-c3ccc4c(ccc5ccc6ccccc6c54)c3)cc2)c2cccc3sc4c5ccccc5ccc4c23)cc1 ; c1ccc(N(c2ccc(-c3cc4ccc5ccccc5c4c4ccccc34)cc2)c2ccc3c(c2)sc2ccc4ccccc4c23)cc1 ; c1ccc(N(c2ccc(-c3cccc4c3ccc3ccc5ccccc5c34)cc2)c2ccc3oc4c5ccccc5ccc4c3c2)cc1]

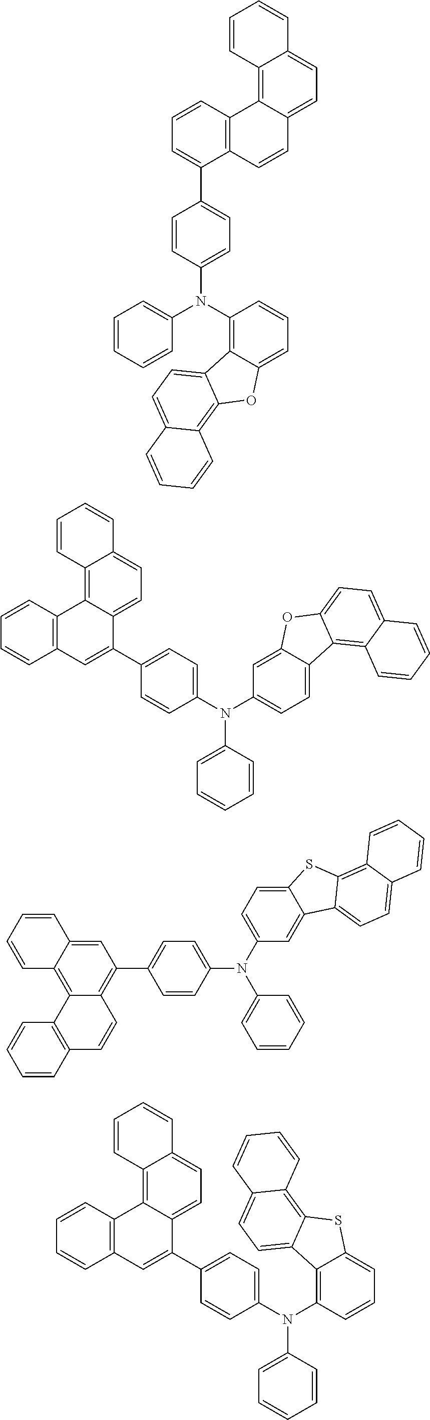 [SMILES: c1ccc(N(c2ccc(-c3cccc4c3ccc3ccc5ccccc5c34)cc2)c2cccc3oc4c5ccccc5ccc4c23)cc1 ; c1ccc(N(c2ccc(-c3cc4ccccc4c4c3ccc3ccccc34)cc2)c2ccc3c(c2)oc2ccc4ccccc4c23)cc1 ; c1ccc(N(c2ccc(-c3cc4ccccc4c4c3ccc3ccccc34)cc2)c2ccc3sc4c5ccccc5ccc4c3c2)cc1 ; c1ccc(N(c2ccc(-c3cc4ccccc4c4c3ccc3ccccc34)cc2)c2cccc3sc4c5ccccc5ccc4c23)cc1]

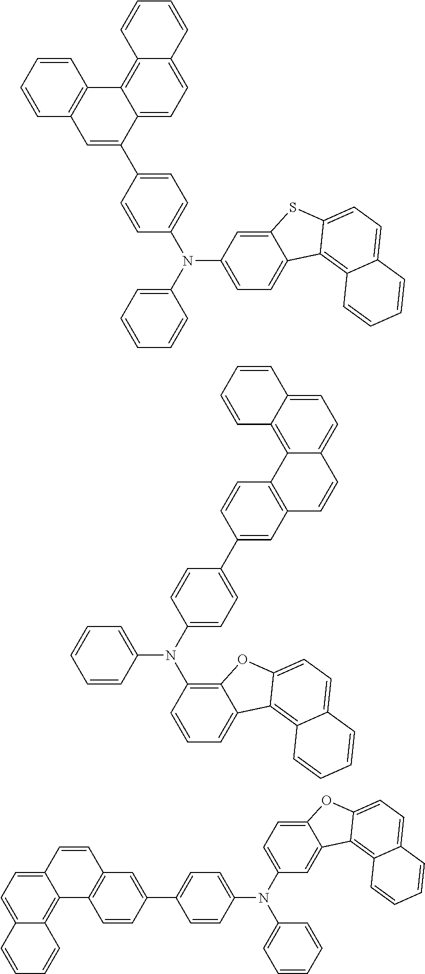

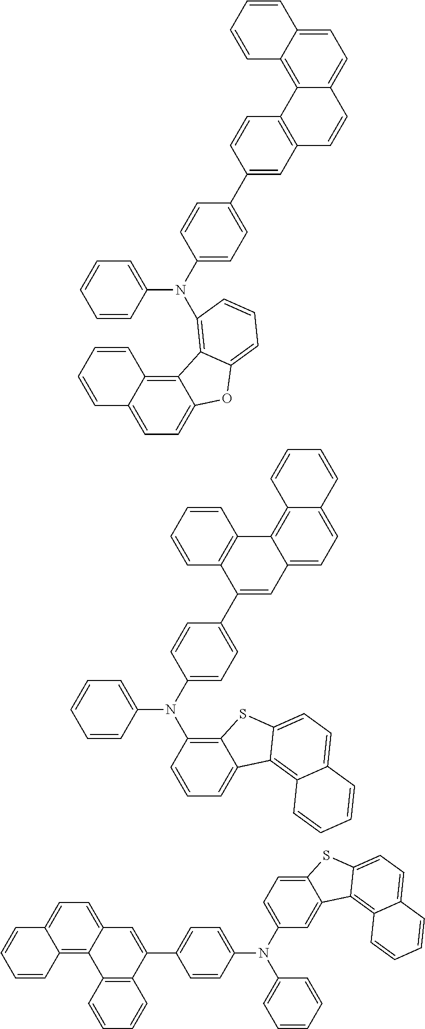

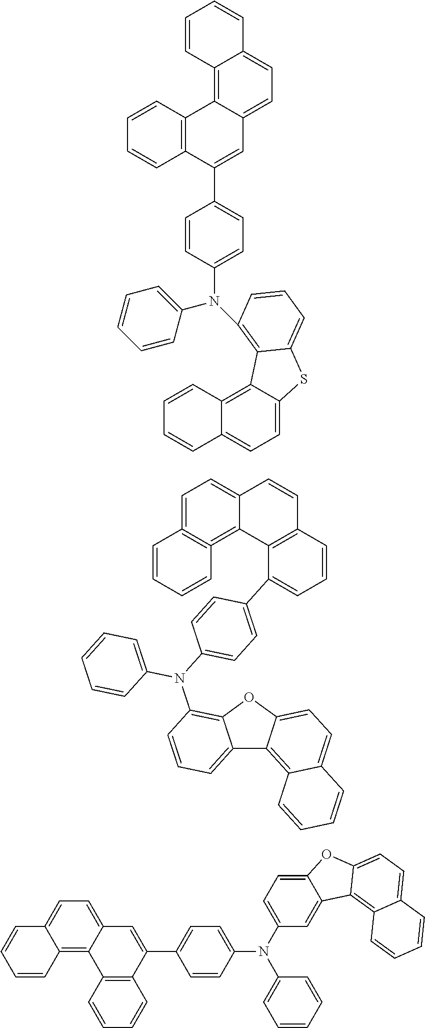

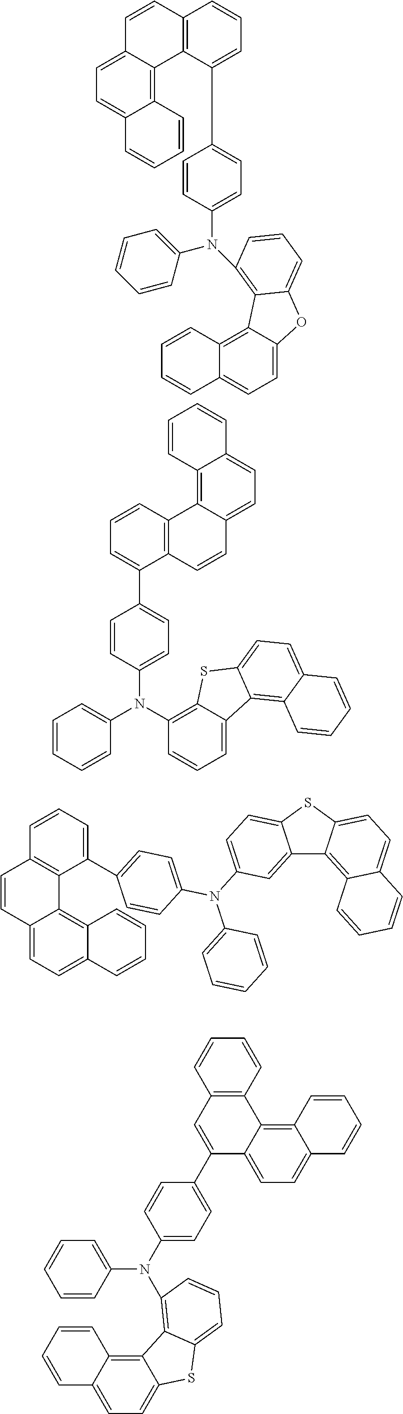

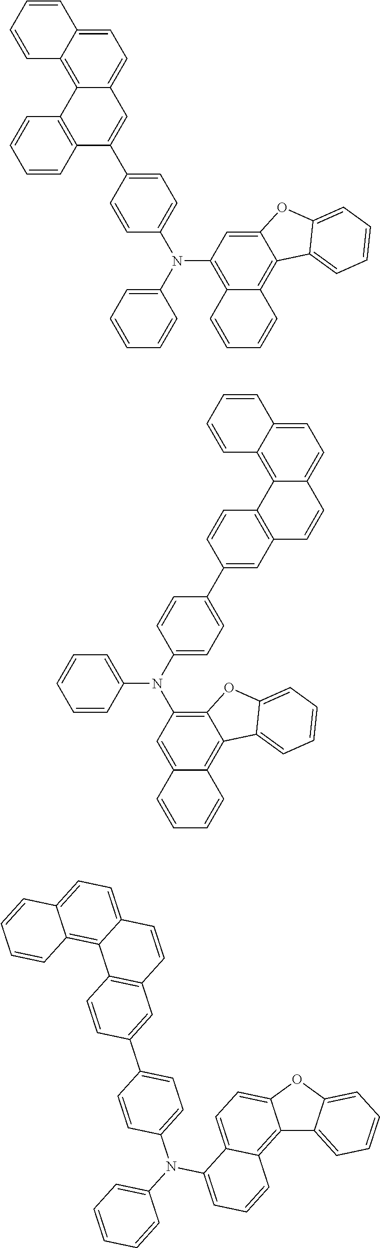 [SMILES: c1ccc(N(c2ccc(-c3cc4ccc5ccccc5c4c4ccccc34)cc2)c2cc3oc4ccccc4c3c3ccccc23)cc1 ; c1ccc(N(c2ccc(-c3ccc4c(ccc5ccc6ccccc6c54)c3)cc2)c2cc3ccccc3c3c2oc2ccccc23)cc1 ; c1ccc(N(c2ccc(-c3ccc4c(ccc5ccc6ccccc6c54)c3)cc2)c2cccc3c2ccc2oc4ccccc4c23)cc1]

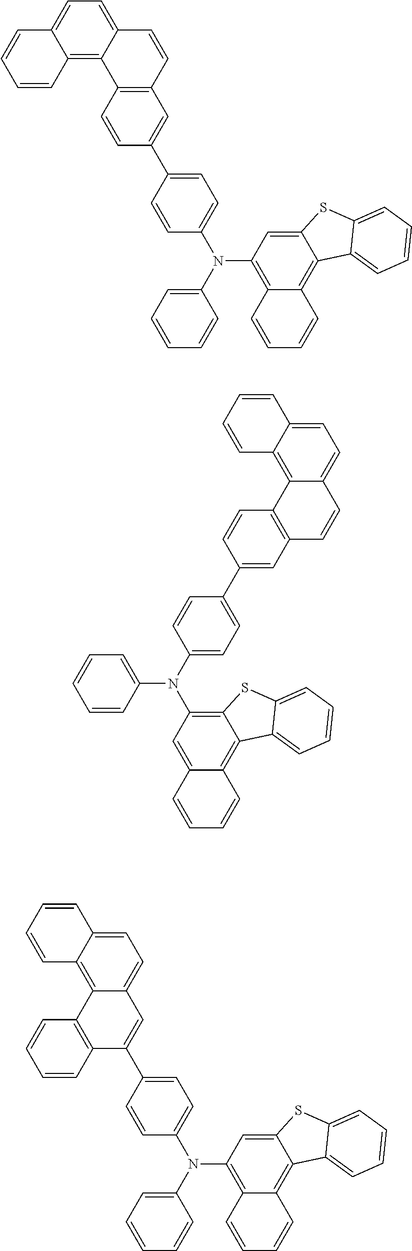 [SMILES: c1ccc(N(c2ccc(-c3ccc4c(ccc5ccc6ccccc6c54)c3)cc2)c2cc3sc4ccccc4c3c3ccccc23)cc1 ; c1ccc(N(c2ccc(-c3ccc4c(ccc5ccc6ccccc6c54)c3)cc2)c2cc3ccccc3c3c2sc2ccccc23)cc1 ; c1ccc(N(c2ccc(-c3cc4ccc5ccccc5c4c4ccccc34)cc2)c2cc3sc4ccccc4c3c3ccccc23)cc1]

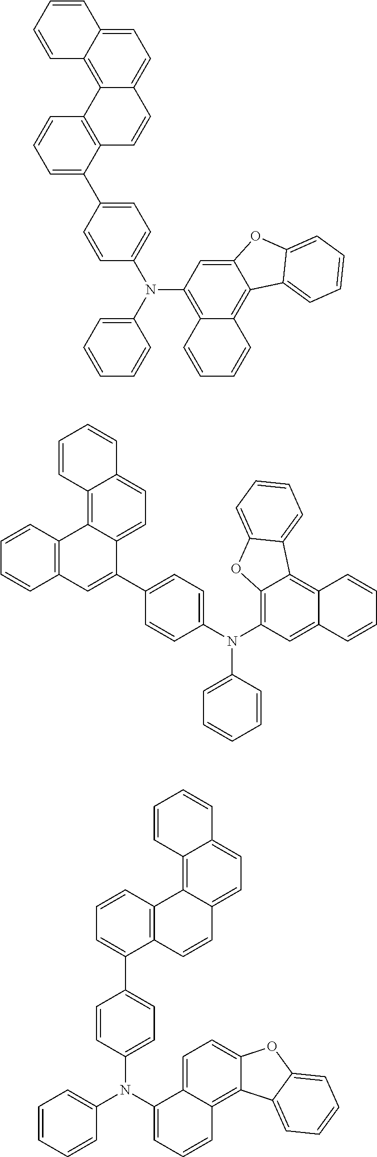 [SMILES: c1ccc(N(c2ccc(-c3cccc4c3ccc3ccc5ccccc5c34)cc2)c2cc3oc4ccccc4c3c3ccccc23)cc1 ; c1ccc(N(c2ccc(-c3cc4ccccc4c4c3ccc3ccccc34)cc2)c2cc3ccccc3c3c2oc2ccccc23)cc1 ; c1ccc(N(c2ccc(-c3cccc4c3ccc3ccc5ccccc5c34)cc2)c2cccc3c2ccc2oc4ccccc4c23)cc1]

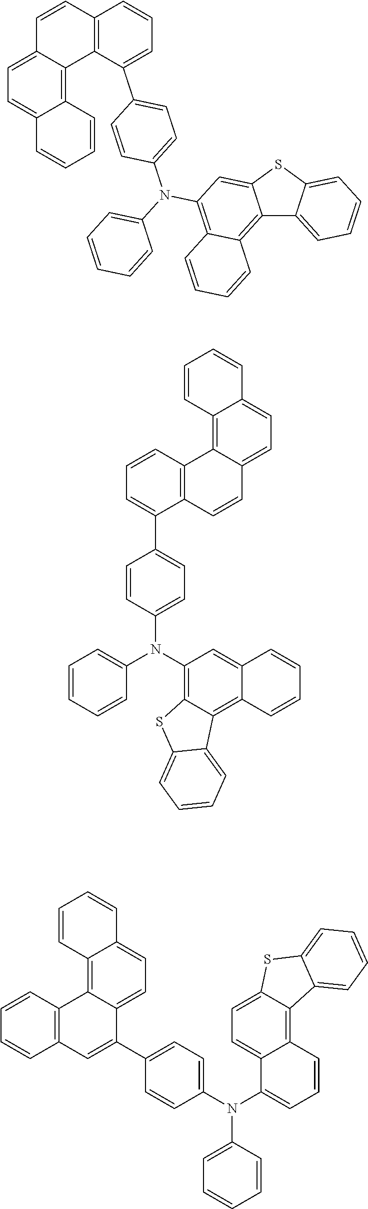 [SMILES: c1ccc(N(c2ccc(-c3cccc4ccc5ccc6ccccc6c5c34)cc2)c2cc3sc4ccccc4c3c3ccccc23)cc1 ; c1ccc(N(c2ccc(-c3cccc4c3ccc3ccc5ccccc5c34)cc2)c2cc3ccccc3c3c2sc2ccccc23)cc1 ; c1ccc(N(c2ccc(-c3cc4ccccc4c4c3ccc3ccccc34)cc2)c2cccc3c2ccc2sc4ccccc4c23)cc1]

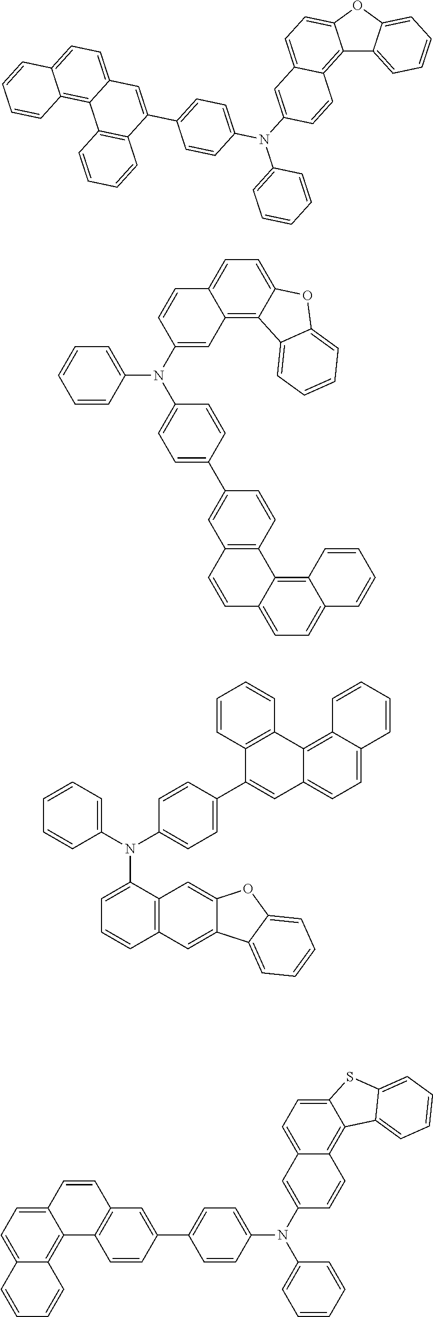

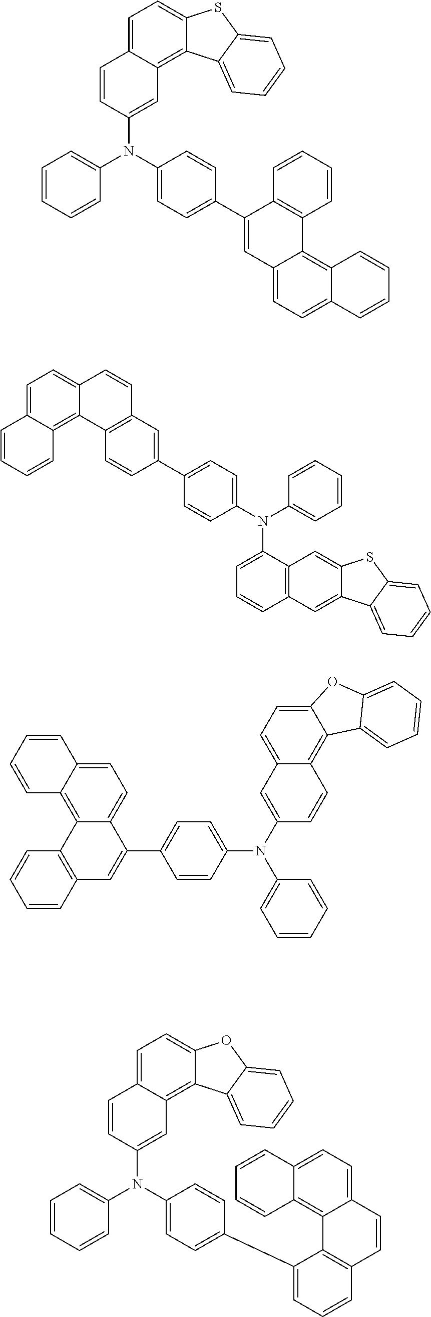 [SMILES: c1ccc(N(c2ccc(-c3cc4ccc5ccccc5c4c4ccccc34)cc2)c2ccc3ccc4sc5ccccc5c4c3c2)cc1 ; c1ccc(N(c2ccc(-c3ccc4c(ccc5ccc6ccccc6c54)c3)cc2)c2cccc3cc4c(cc23)sc2ccccc24)cc1 ; c1ccc(N(c2ccc(-c3cc4ccccc4c4c3ccc3ccccc34)cc2)c2ccc3c(ccc4oc5ccccc5c43)c2)cc1 ; c1ccc(N(c2ccc(-c3cccc4ccc5ccc6ccccc6c5c34)cc2)c2ccc3ccc4oc5ccccc5c4c3c2)cc1]

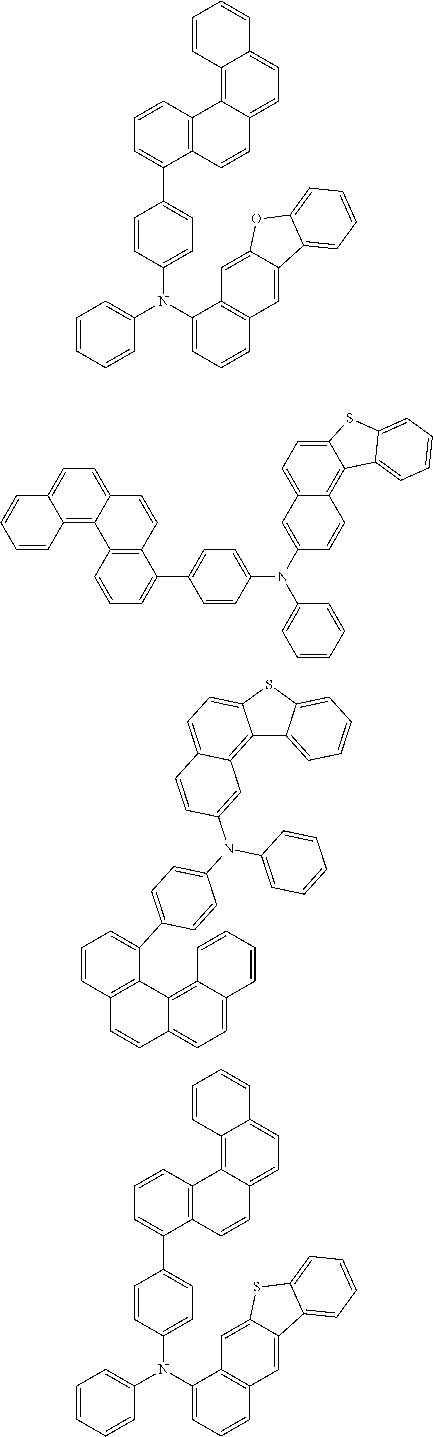

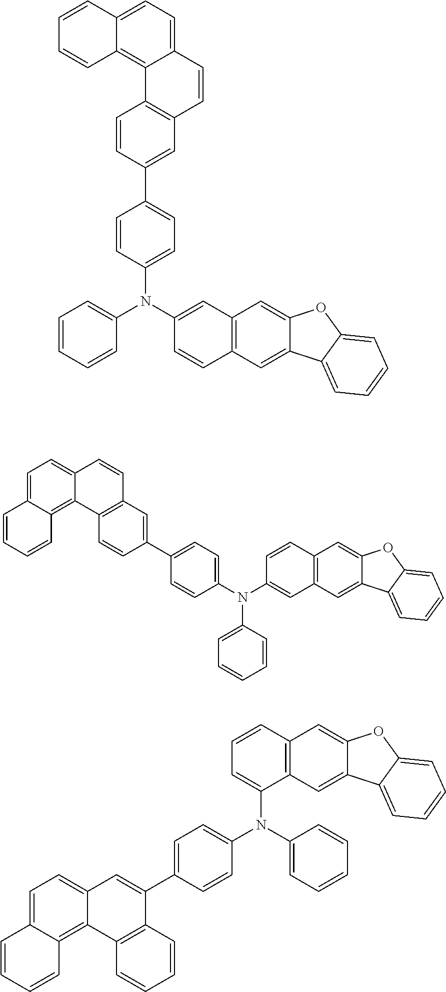 [SMILES: c1ccc(N(c2ccc(-c3ccc4c(ccc5ccc6ccccc6c54)c3)cc2)c2ccc3cc4c(cc3c2)oc2ccccc24)cc1 ; c1ccc(N(c2ccc(-c3ccc4c(ccc5ccc6ccccc6c54)c3)cc2)c2ccc3cc4oc5ccccc5c4cc3c2)cc1 ; c1ccc(N(c2ccc(-c3cc4ccc5ccccc5c4c4ccccc34)cc2)c2cccc3cc4oc5ccccc5c4cc23)cc1]

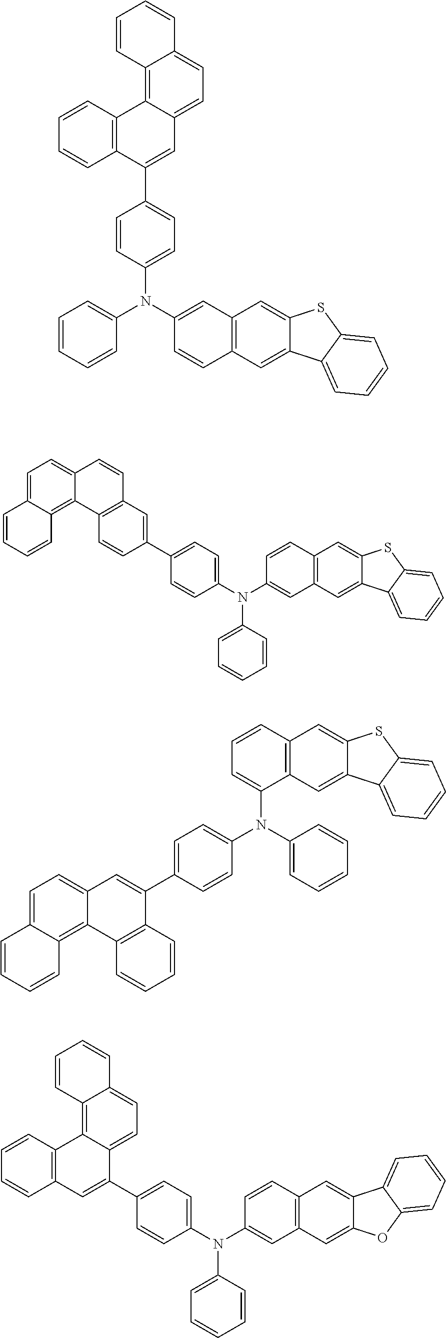 [SMILES: c1ccc(N(c2ccc(-c3cc4ccc5ccccc5c4c4ccccc34)cc2)c2ccc3cc4c(cc3c2)sc2ccccc24)cc1 ; c1ccc(N(c2ccc(-c3ccc4c(ccc5ccc6ccccc6c54)c3)cc2)c2ccc3cc4sc5ccccc5c4cc3c2)cc1 ; c1ccc(N(c2ccc(-c3cc4ccc5ccccc5c4c4ccccc34)cc2)c2cccc3cc4sc5ccccc5c4cc23)cc1 ; c1ccc(N(c2ccc(-c3cc4ccccc4c4c3ccc3ccccc34)cc2)c2ccc3cc4c(cc3c2)oc2ccccc24)cc1]

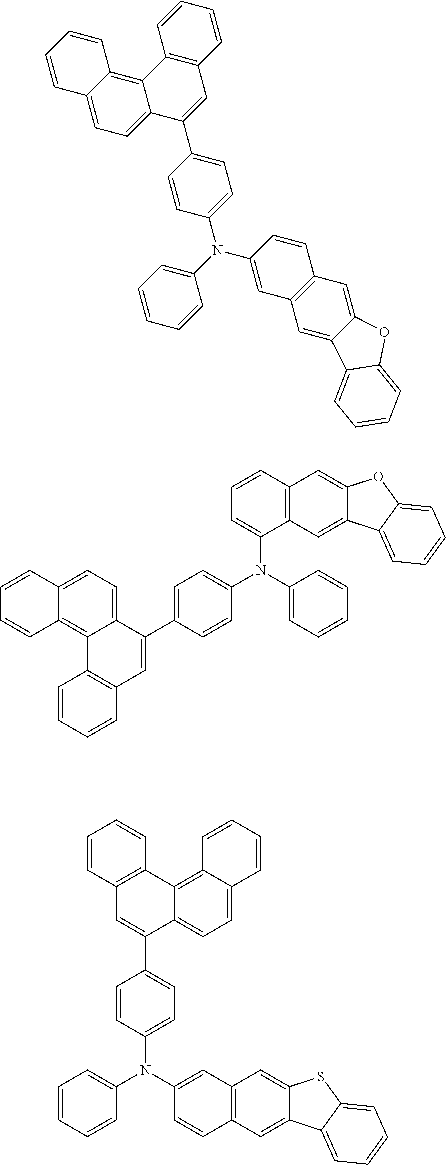 [SMILES: c1ccc(N(c2ccc(-c3cc4ccccc4c4c3ccc3ccccc34)cc2)c2ccc3cc4oc5ccccc5c4cc3c2)cc1 ; c1ccc(N(c2ccc(-c3cc4ccccc4c4c3ccc3ccccc34)cc2)c2cccc3cc4oc5ccccc5c4cc23)cc1 ; c1ccc(N(c2ccc(-c3cc4ccccc4c4c3ccc3ccccc34)cc2)c2ccc3cc4c(cc3c2)sc2ccccc24)cc1]

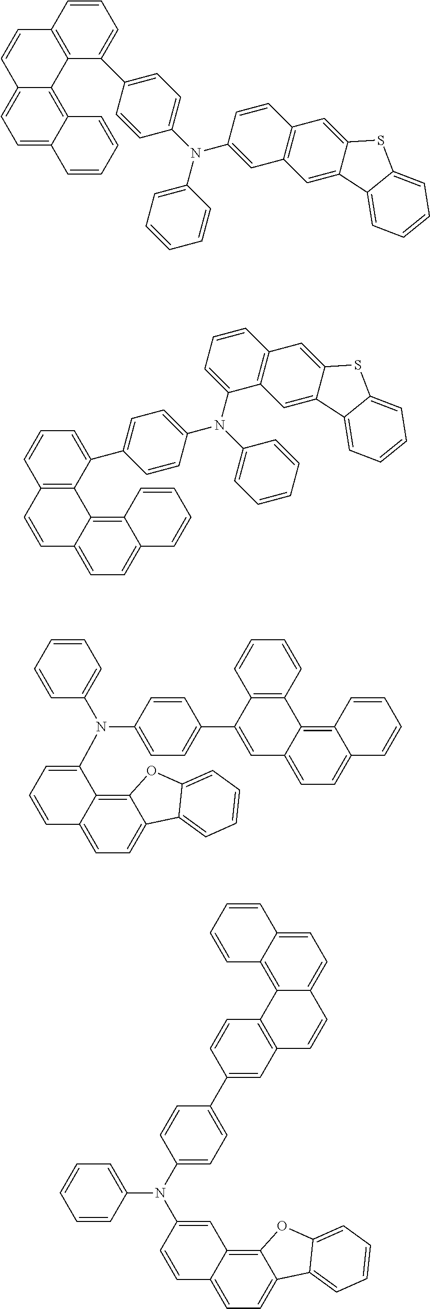 [SMILES: c1ccc(N(c2ccc(-c3cccc4ccc5ccc6ccccc6c5c34)cc2)c2ccc3cc4sc5ccccc5c4cc3c2)cc1 ; c1ccc(N(c2ccc(-c3cccc4ccc5ccc6ccccc6c5c34)cc2)c2cccc3cc4sc5ccccc5c4cc23)cc1 ; c1ccc(N(c2ccc(-c3cc4ccc5ccccc5c4c4ccccc34)cc2)c2cccc3ccc4c5ccccc5oc4c23)cc1 ; c1ccc(N(c2ccc(-c3ccc4c(ccc5ccc6ccccc6c54)c3)cc2)c2ccc3ccc4c5ccccc5oc4c3c2)cc1]

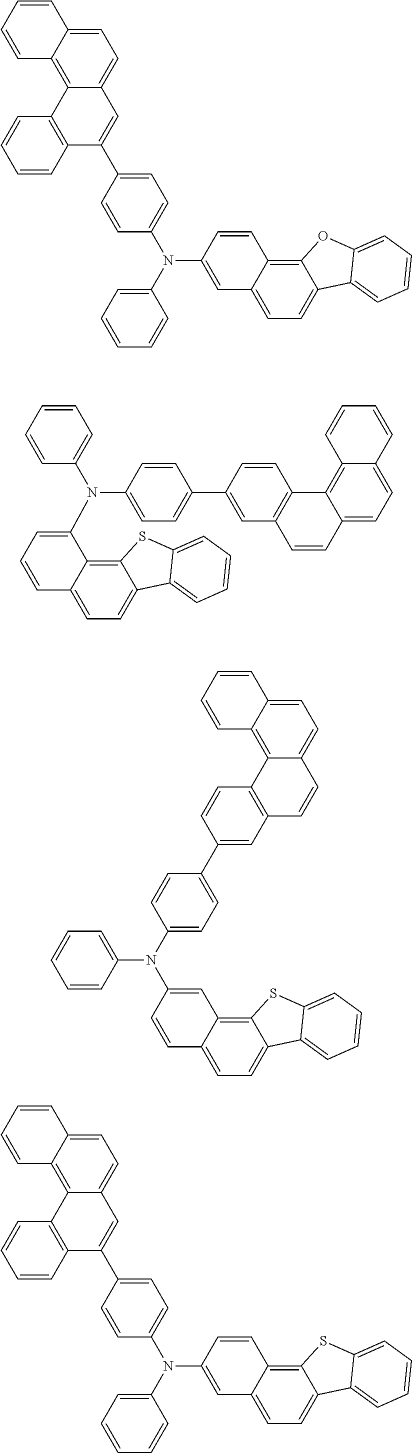 [SMILES: c1ccc(N(c2ccc(-c3cc4ccc5ccccc5c4c4ccccc34)cc2)c2ccc3c(ccc4c5ccccc5oc34)c2)cc1 ; c1ccc(N(c2ccc(-c3ccc4c(ccc5ccc6ccccc6c54)c3)cc2)c2cccc3ccc4c5ccccc5sc4c23)cc1 ; c1ccc(N(c2ccc(-c3ccc4c(ccc5ccc6ccccc6c54)c3)cc2)c2ccc3ccc4c5ccccc5sc4c3c2)cc1 ; c1ccc(N(c2ccc(-c3cc4ccc5ccccc5c4c4ccccc34)cc2)c2ccc3c(ccc4c5ccccc5sc34)c2)cc1]

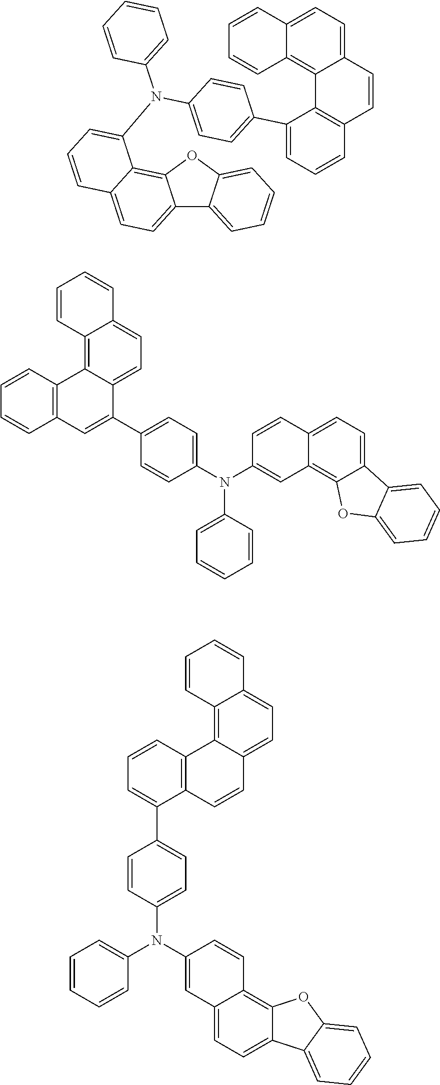 [SMILES: c1ccc(N(c2ccc(-c3cccc4ccc5ccc6ccccc6c5c34)cc2)c2cccc3ccc4c5ccccc5oc4c23)cc1 ; c1ccc(N(c2ccc(-c3cc4ccccc4c4c3ccc3ccccc34)cc2)c2ccc3ccc4c5ccccc5oc4c3c2)cc1 ; c1ccc(N(c2ccc(-c3cccc4c3ccc3ccc5ccccc5c34)cc2)c2ccc3c(ccc4c5ccccc5oc34)c2)cc1]

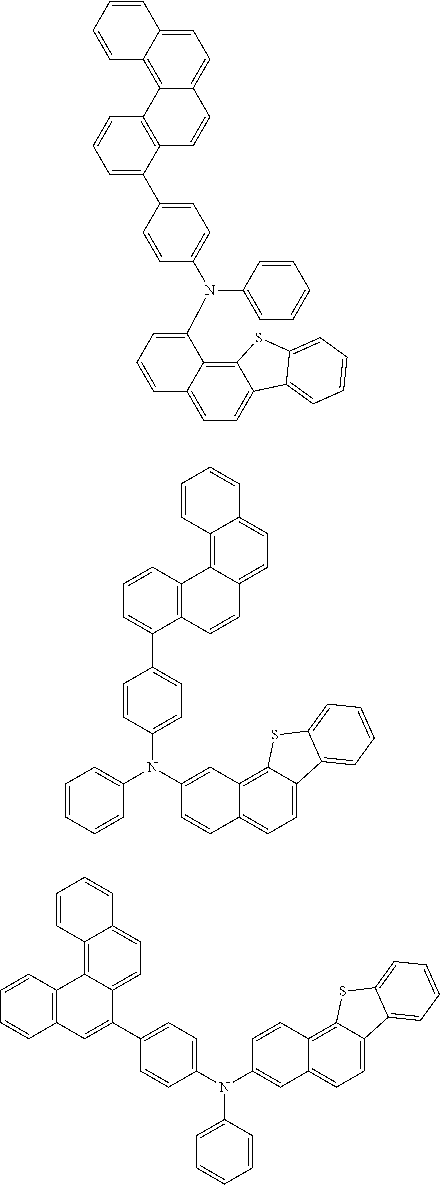

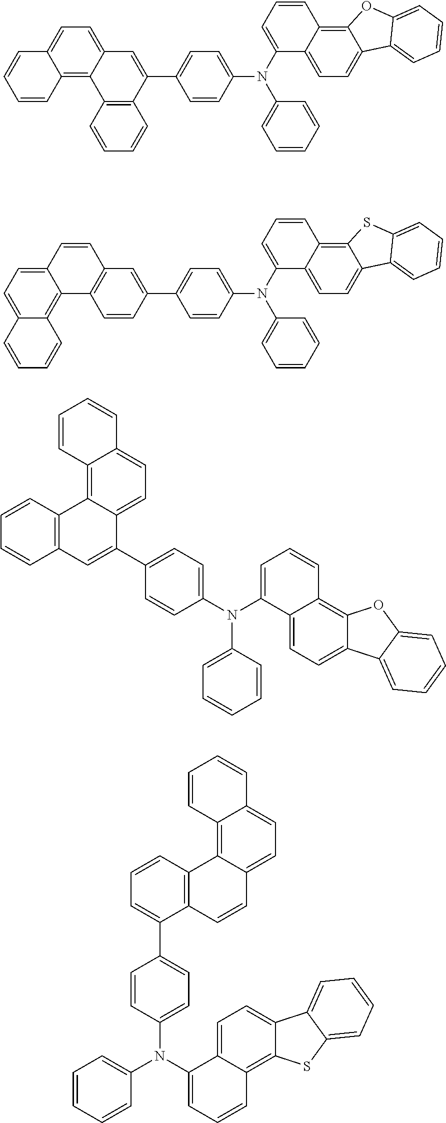

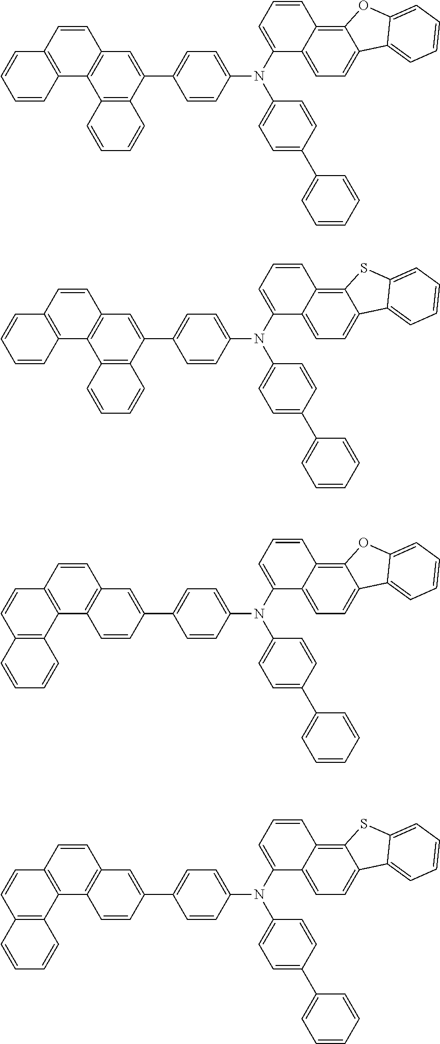 [SMILES: c1ccc(-c2ccc(N(c3ccc(-c4cc5ccc6ccccc6c5c5ccccc45)cc3)c3cccc4c3ccc3c5ccccc5oc43)cc2)cc1 ; c1ccc(-c2ccc(N(c3ccc(-c4cc5ccc6ccccc6c5c5ccccc45)cc3)c3cccc4c3ccc3c5ccccc5sc43)cc2)cc1 ; c1ccc(-c2ccc(N(c3ccc(-c4ccc5c(ccc6ccc7ccccc7c65)c4)cc3)c3cccc4c3ccc3c5ccccc5oc43)cc2)cc1 ; c1ccc(-c2ccc(N(c3ccc(-c4ccc5c(ccc6ccc7ccccc7c65)c4)cc3)c3cccc4c3ccc3c5ccccc5sc43)cc2)cc1]

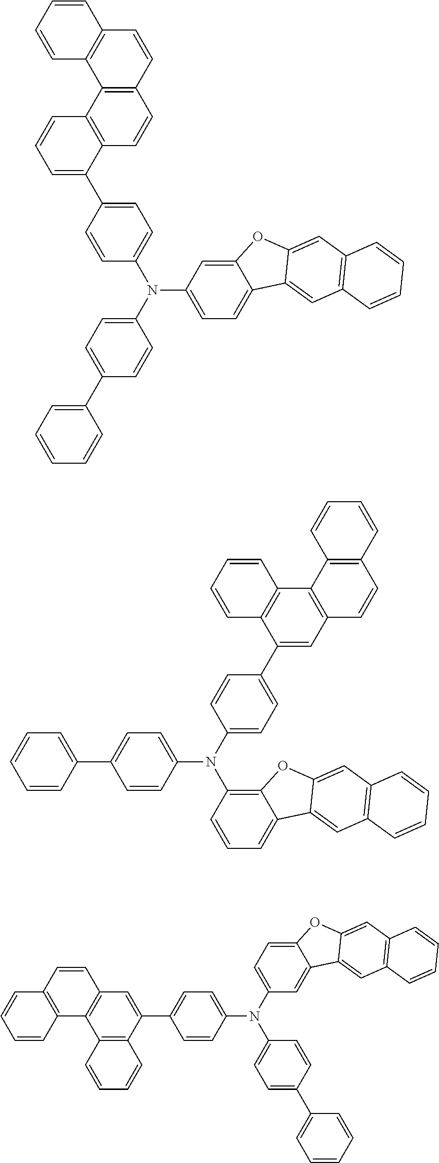 [SMILES: c1ccc(-c2ccc(N(c3ccc(-c4cccc5c4ccc4ccc6ccccc6c45)cc3)c3ccc4c(c3)oc3cc5ccccc5cc34)cc2)cc1 ; c1ccc(-c2ccc(N(c3ccc(-c4cc5ccc6ccccc6c5c5ccccc45)cc3)c3cccc4c3oc3cc5ccccc5cc34)cc2)cc1 ; c1ccc(-c2ccc(N(c3ccc(-c4cc5ccc6ccccc6c5c5ccccc45)cc3)c3ccc4oc5cc6ccccc6cc5c4c3)cc2)cc1]

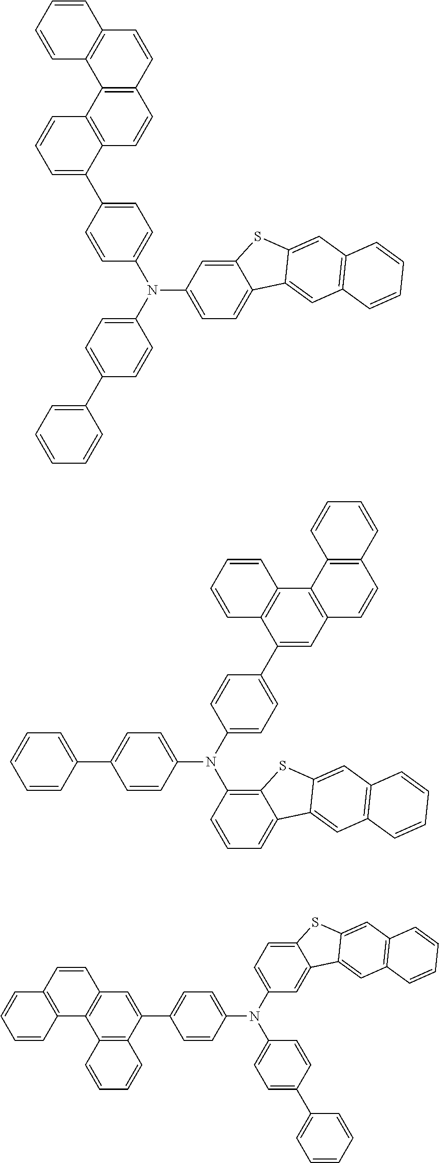 [SMILES: c1ccc(-c2ccc(N(c3ccc(-c4cccc5c4ccc4ccc6ccccc6c45)cc3)c3ccc4c(c3)sc3cc5ccccc5cc34)cc2)cc1 ; c1ccc(-c2ccc(N(c3ccc(-c4cc5ccc6ccccc6c5c5ccccc45)cc3)c3cccc4c3sc3cc5ccccc5cc34)cc2)cc1 ; c1ccc(-c2ccc(N(c3ccc(-c4cc5ccc6ccccc6c5c5ccccc45)cc3)c3ccc4sc5cc6ccccc6cc5c4c3)cc2)cc1]

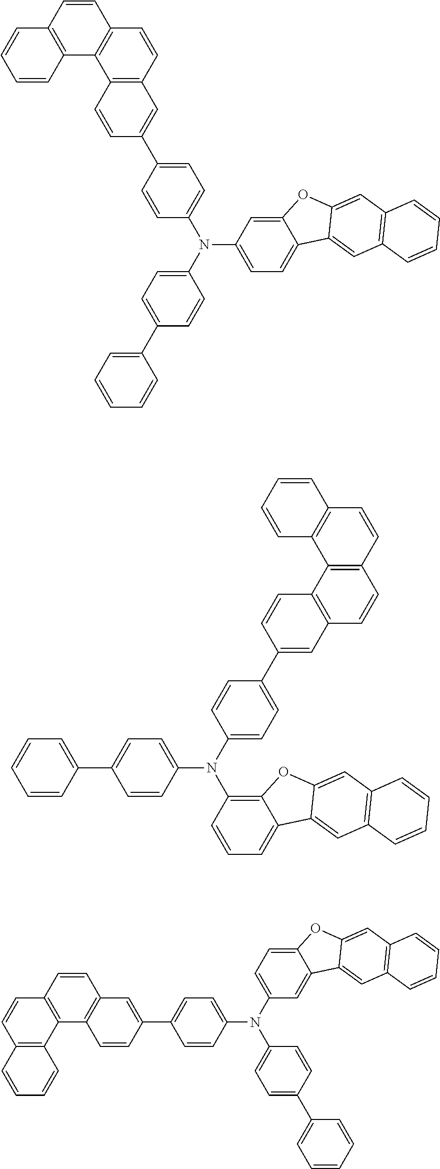 [SMILES: c1ccc(-c2ccc(N(c3ccc(-c4ccc5c(ccc6ccc7ccccc7c65)c4)cc3)c3ccc4c(c3)oc3cc5ccccc5cc34)cc2)cc1 ; c1ccc(-c2ccc(N(c3ccc(-c4ccc5c(ccc6ccc7ccccc7c65)c4)cc3)c3cccc4c3oc3cc5ccccc5cc34)cc2)cc1 ; c1ccc(-c2ccc(N(c3ccc(-c4ccc5c(ccc6ccc7ccccc7c65)c4)cc3)c3ccc4oc5cc6ccccc6cc5c4c3)cc2)cc1]

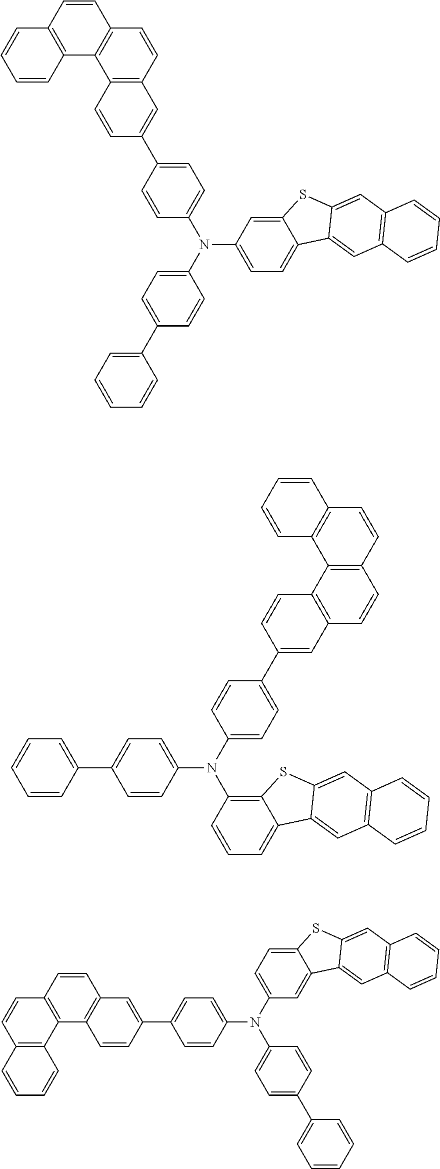 [SMILES: c1ccc(-c2ccc(N(c3ccc(-c4ccc5c(ccc6ccc7ccccc7c65)c4)cc3)c3ccc4c(c3)sc3cc5ccccc5cc34)cc2)cc1 ; c1ccc(-c2ccc(N(c3ccc(-c4ccc5c(ccc6ccc7ccccc7c65)c4)cc3)c3cccc4c3sc3cc5ccccc5cc34)cc2)cc1 ; c1ccc(-c2ccc(N(c3ccc(-c4ccc5c(ccc6ccc7ccccc7c65)c4)cc3)c3ccc4sc5cc6ccccc6cc5c4c3)cc2)cc1]

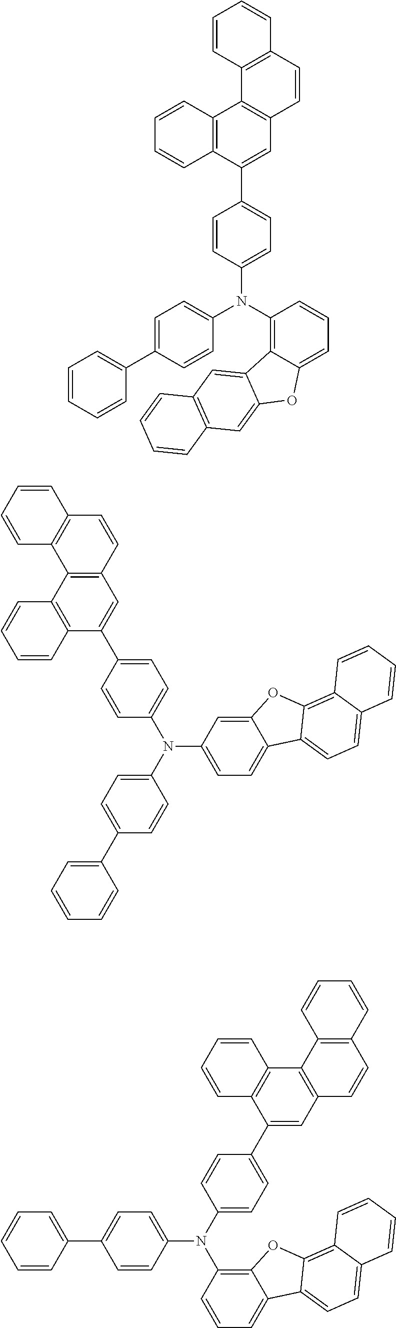 [SMILES: c1ccc(-c2ccc(N(c3ccc(-c4cc5ccc6ccccc6c5c5ccccc45)cc3)c3cccc4oc5cc6ccccc6cc5c34)cc2)cc1 ; c1ccc(-c2ccc(N(c3ccc(-c4cc5ccc6ccccc6c5c5ccccc45)cc3)c3ccc4c(c3)oc3c5ccccc5ccc43)cc2)cc1 ; c1ccc(-c2ccc(N(c3ccc(-c4cc5ccc6ccccc6c5c5ccccc45)cc3)c3cccc4c3oc3c5ccccc5ccc43)cc2)cc1]

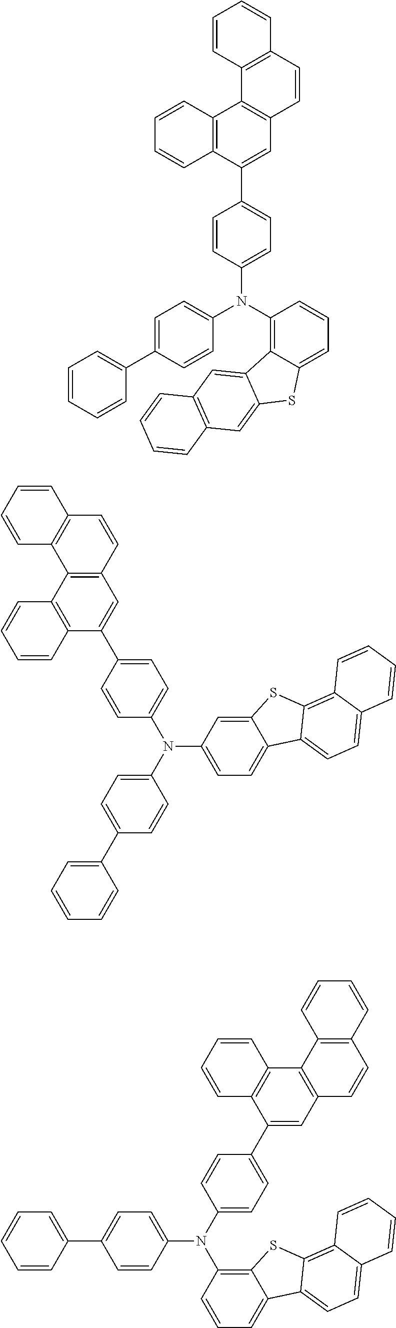 [SMILES: c1ccc(-c2ccc(N(c3ccc(-c4cc5ccc6ccccc6c5c5ccccc45)cc3)c3cccc4sc5cc6ccccc6cc5c34)cc2)cc1 ; c1ccc(-c2ccc(N(c3ccc(-c4cc5ccc6ccccc6c5c5ccccc45)cc3)c3ccc4c(c3)sc3c5ccccc5ccc43)cc2)cc1 ; c1ccc(-c2ccc(N(c3ccc(-c4cc5ccc6ccccc6c5c5ccccc45)cc3)c3cccc4c3sc3c5ccccc5ccc43)cc2)cc1]

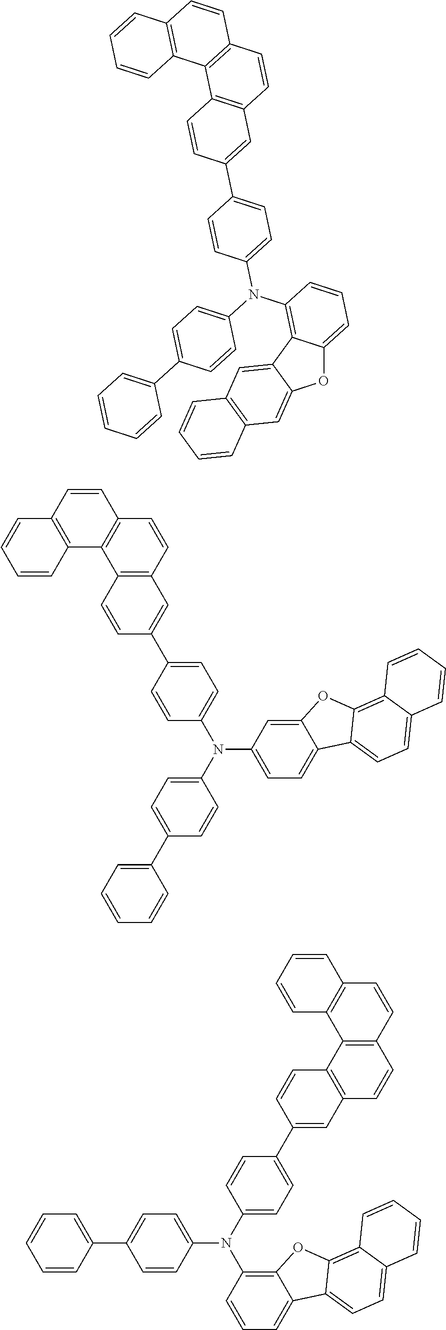 [SMILES: c1ccc(-c2ccc(N(c3ccc(-c4ccc5c(ccc6ccc7ccccc7c65)c4)cc3)c3cccc4oc5cc6ccccc6cc5c34)cc2)cc1 ; c1ccc(-c2ccc(N(c3ccc(-c4ccc5c(ccc6ccc7ccccc7c65)c4)cc3)c3ccc4c(c3)oc3c5ccccc5ccc43)cc2)cc1 ; c1ccc(-c2ccc(N(c3ccc(-c4ccc5c(ccc6ccc7ccccc7c65)c4)cc3)c3cccc4c3oc3c5ccccc5ccc43)cc2)cc1]

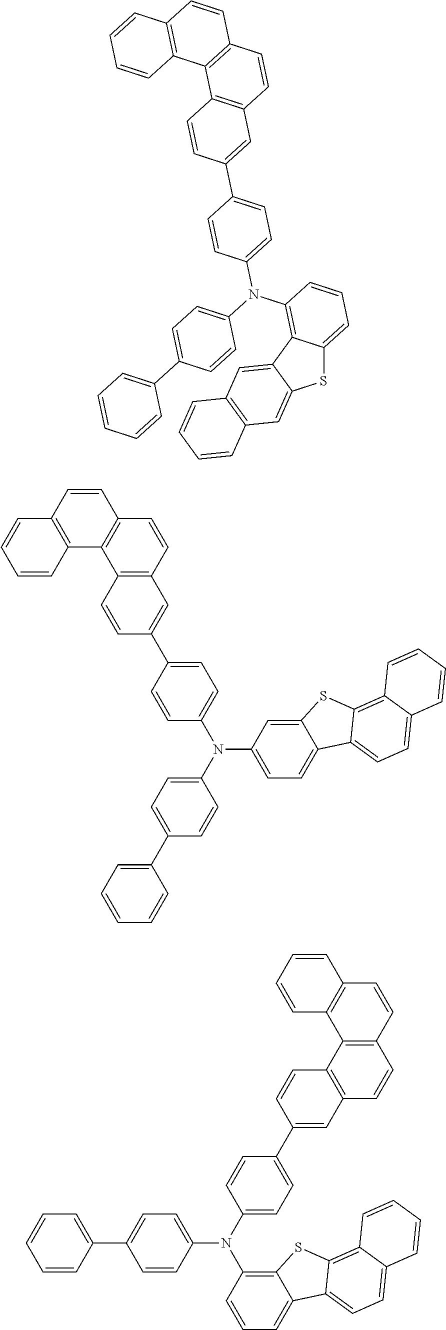 [SMILES: c1ccc(-c2ccc(N(c3ccc(-c4ccc5c(ccc6ccc7ccccc7c65)c4)cc3)c3cccc4sc5cc6ccccc6cc5c34)cc2)cc1 ; c1ccc(-c2ccc(N(c3ccc(-c4ccc5c(ccc6ccc7ccccc7c65)c4)cc3)c3ccc4c(c3)sc3c5ccccc5ccc43)cc2)cc1 ; c1ccc(-c2ccc(N(c3ccc(-c4ccc5c(ccc6ccc7ccccc7c65)c4)cc3)c3cccc4c3sc3c5ccccc5ccc43)cc2)cc1]

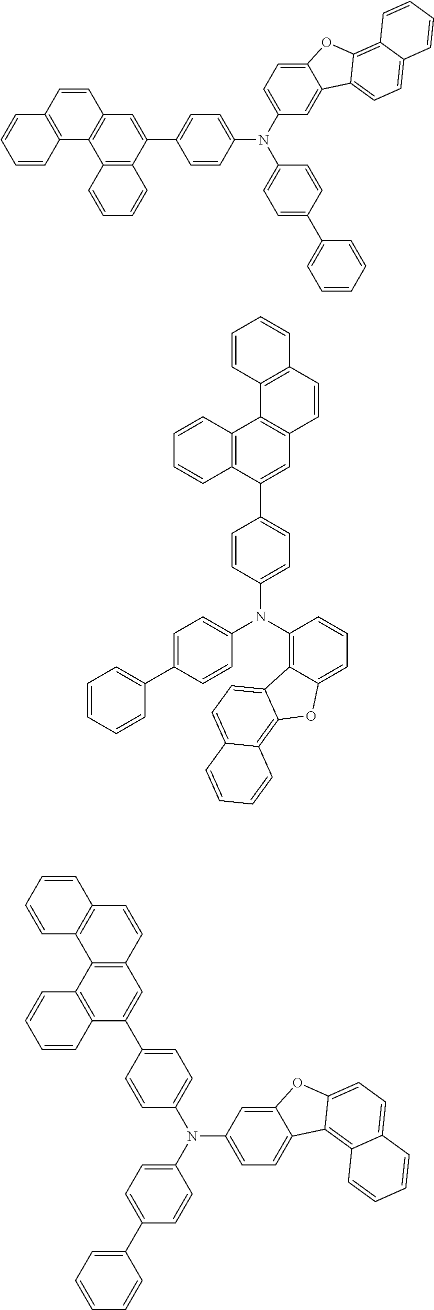 [SMILES: c1ccc(-c2ccc(N(c3ccc(-c4cc5ccc6ccccc6c5c5ccccc45)cc3)c3ccc4oc5c6ccccc6ccc5c4c3)cc2)cc1 ; c1ccc(-c2ccc(N(c3ccc(-c4cc5ccc6ccccc6c5c5ccccc45)cc3)c3cccc4oc5c6ccccc6ccc5c34)cc2)cc1 ; c1ccc(-c2ccc(N(c3ccc(-c4cc5ccc6ccccc6c5c5ccccc45)cc3)c3ccc4c(c3)oc3ccc5ccccc5c34)cc2)cc1]

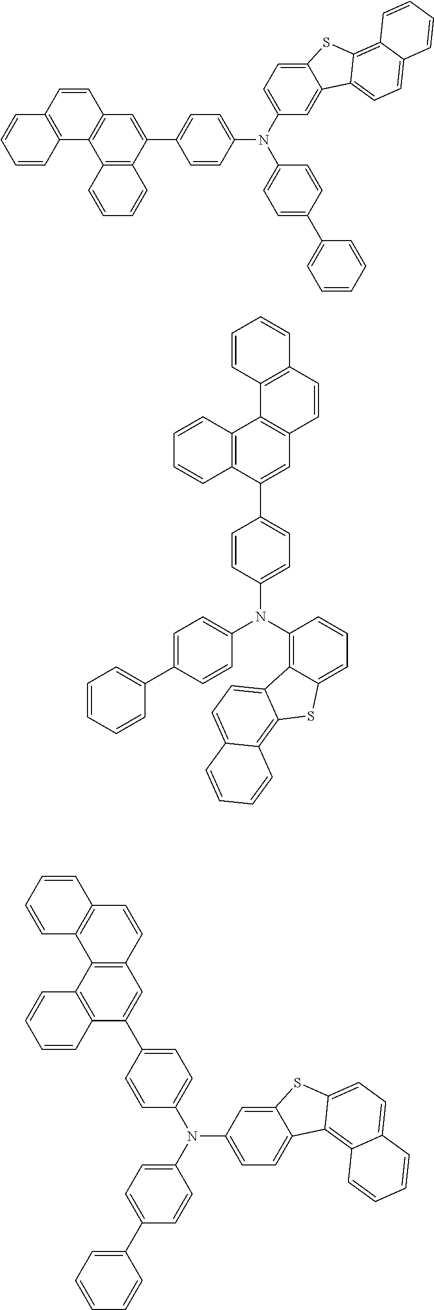 [SMILES: c1ccc(-c2ccc(N(c3ccc(-c4cc5ccc6ccccc6c5c5ccccc45)cc3)c3ccc4sc5c6ccccc6ccc5c4c3)cc2)cc1 ; c1ccc(-c2ccc(N(c3ccc(-c4cc5ccc6ccccc6c5c5ccccc45)cc3)c3cccc4sc5c6ccccc6ccc5c34)cc2)cc1 ; c1ccc(-c2ccc(N(c3ccc(-c4cc5ccc6ccccc6c5c5ccccc45)cc3)c3ccc4c(c3)sc3ccc5ccccc5c34)cc2)cc1]

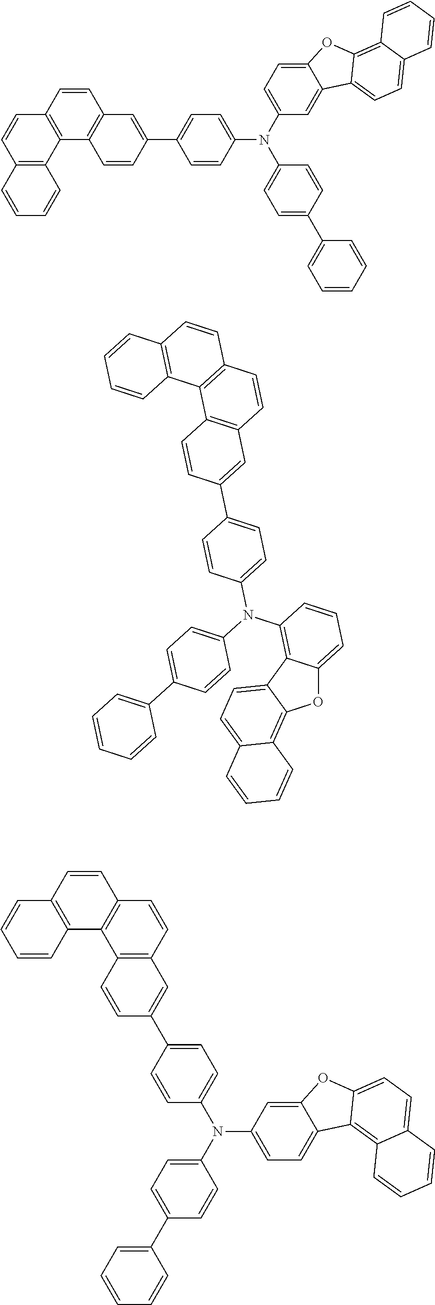 [SMILES: c1ccc(-c2ccc(N(c3ccc(-c4ccc5c(ccc6ccc7ccccc7c65)c4)cc3)c3ccc4oc5c6ccccc6ccc5c4c3)cc2)cc1 ; c1ccc(-c2ccc(N(c3ccc(-c4ccc5c(ccc6ccc7ccccc7c65)c4)cc3)c3cccc4oc5c6ccccc6ccc5c34)cc2)cc1 ; c1ccc(-c2ccc(N(c3ccc(-c4ccc5c(ccc6ccc7ccccc7c65)c4)cc3)c3ccc4c(c3)oc3ccc5ccccc5c34)cc2)cc1]

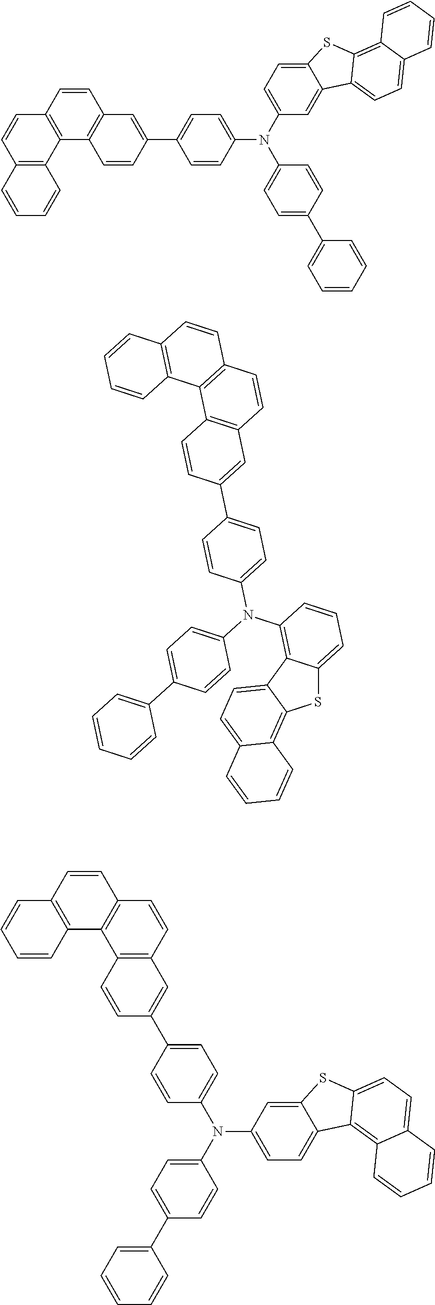 [SMILES: c1ccc(-c2ccc(N(c3ccc(-c4ccc5c(ccc6ccc7ccccc7c65)c4)cc3)c3ccc4sc5c6ccccc6ccc5c4c3)cc2)cc1 ; c1ccc(-c2ccc(N(c3ccc(-c4ccc5c(ccc6ccc7ccccc7c65)c4)cc3)c3cccc4sc5c6ccccc6ccc5c34)cc2)cc1 ; c1ccc(-c2ccc(N(c3ccc(-c4ccc5c(ccc6ccc7ccccc7c65)c4)cc3)c3ccc4c(c3)sc3ccc5ccccc5c34)cc2)cc1]

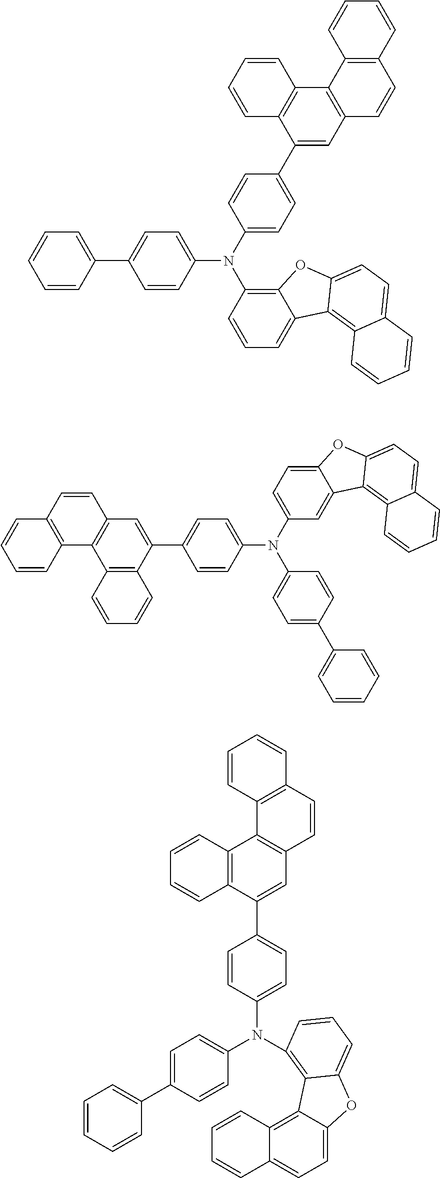 [SMILES: c1ccc(-c2ccc(N(c3ccc(-c4cc5ccc6ccccc6c5c5ccccc45)cc3)c3cccc4c3oc3ccc5ccccc5c34)cc2)cc1 ; c1ccc(-c2ccc(N(c3ccc(-c4cc5ccc6ccccc6c5c5ccccc45)cc3)c3ccc4oc5ccc6ccccc6c5c4c3)cc2)cc1 ; c1ccc(-c2ccc(N(c3ccc(-c4cc5ccc6ccccc6c5c5ccccc45)cc3)c3cccc4oc5ccc6ccccc6c5c34)cc2)cc1]

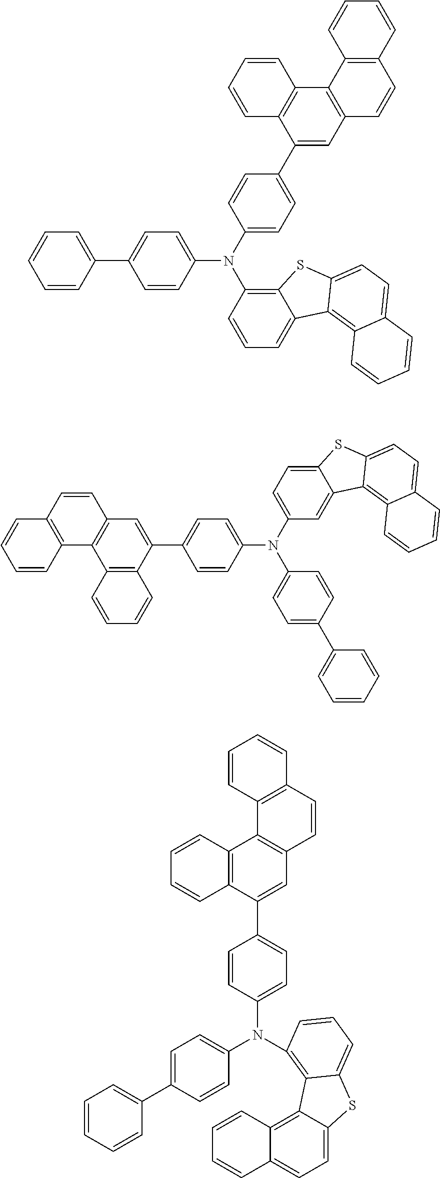 [SMILES: c1ccc(-c2ccc(N(c3ccc(-c4cc5ccc6ccccc6c5c5ccccc45)cc3)c3cccc4c3sc3ccc5ccccc5c34)cc2)cc1 ; c1ccc(-c2ccc(N(c3ccc(-c4cc5ccc6ccccc6c5c5ccccc45)cc3)c3ccc4sc5ccc6ccccc6c5c4c3)cc2)cc1 ; c1ccc(-c2ccc(N(c3ccc(-c4cc5ccc6ccccc6c5c5ccccc45)cc3)c3cccc4sc5ccc6ccccc6c5c34)cc2)cc1]

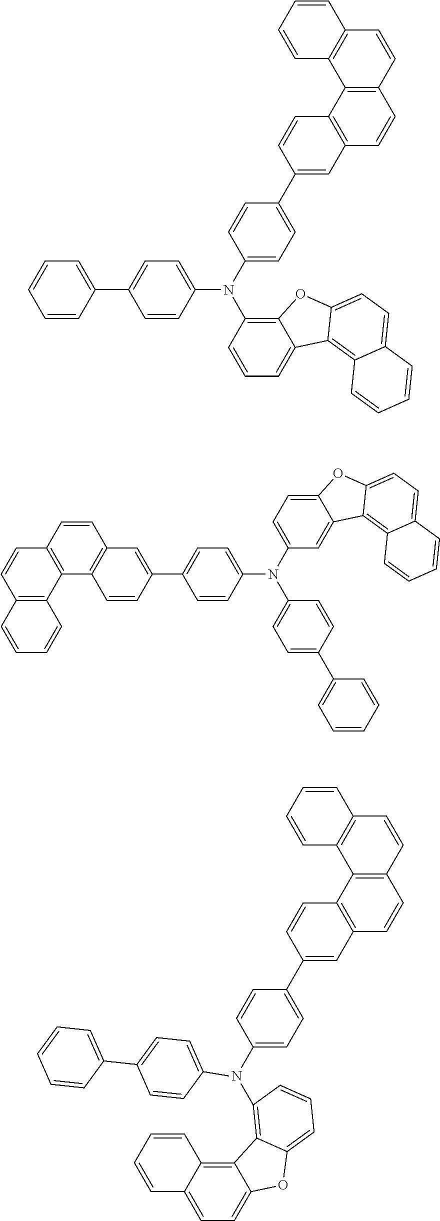 [SMILES: c1ccc(-c2ccc(N(c3ccc(-c4ccc5c(ccc6ccc7ccccc7c65)c4)cc3)c3cccc4c3oc3ccc5ccccc5c34)cc2)cc1 ; c1ccc(-c2ccc(N(c3ccc(-c4ccc5c(ccc6ccc7ccccc7c65)c4)cc3)c3ccc4oc5ccc6ccccc6c5c4c3)cc2)cc1 ; c1ccc(-c2ccc(N(c3ccc(-c4ccc5c(ccc6ccc7ccccc7c65)c4)cc3)c3cccc4oc5ccc6ccccc6c5c34)cc2)cc1]

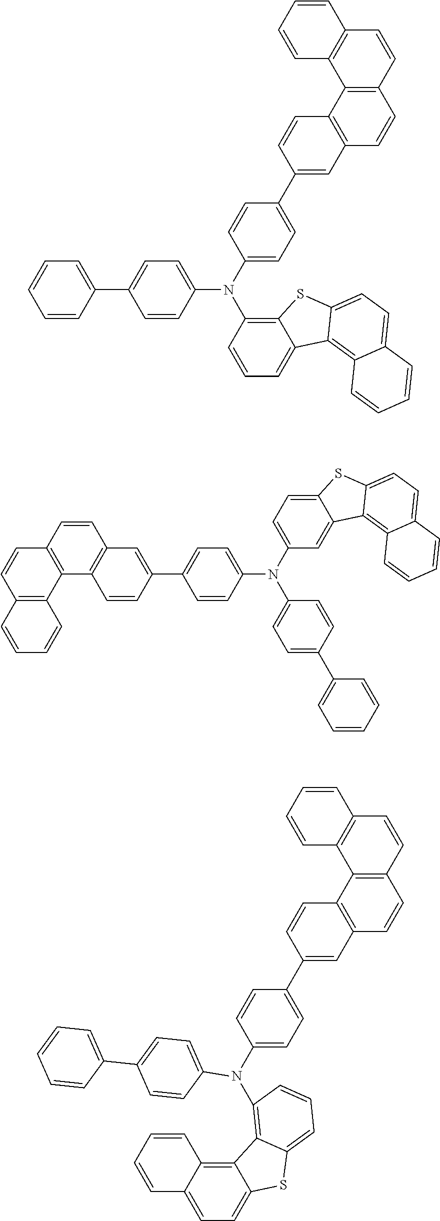 [SMILES: c1ccc(-c2ccc(N(c3ccc(-c4ccc5c(ccc6ccc7ccccc7c65)c4)cc3)c3cccc4c3sc3ccc5ccccc5c34)cc2)cc1 ; c1ccc(-c2ccc(N(c3ccc(-c4ccc5c(ccc6ccc7ccccc7c65)c4)cc3)c3ccc4sc5ccc6ccccc6c5c4c3)cc2)cc1 ; c1ccc(-c2ccc(N(c3ccc(-c4ccc5c(ccc6ccc7ccccc7c65)c4)cc3)c3cccc4sc5ccc6ccccc6c5c34)cc2)cc1]

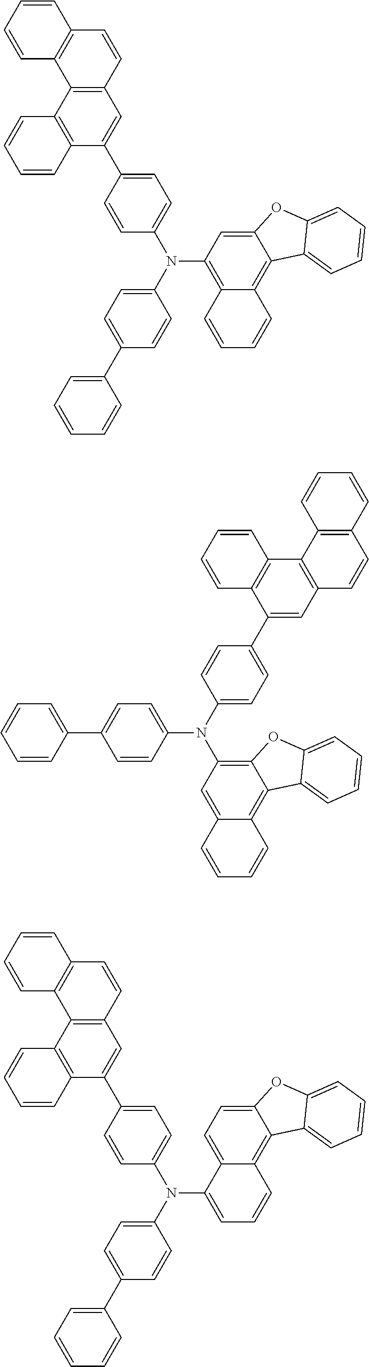 [SMILES: c1ccc(-c2ccc(N(c3ccc(-c4cc5ccc6ccccc6c5c5ccccc45)cc3)c3cc4oc5ccccc5c4c4ccccc34)cc2)cc1 ; c1ccc(-c2ccc(N(c3ccc(-c4cc5ccc6ccccc6c5c5ccccc45)cc3)c3cc4ccccc4c4c3oc3ccccc34)cc2)cc1 ; c1ccc(-c2ccc(N(c3ccc(-c4cc5ccc6ccccc6c5c5ccccc45)cc3)c3cccc4c3ccc3oc5ccccc5c34)cc2)cc1]

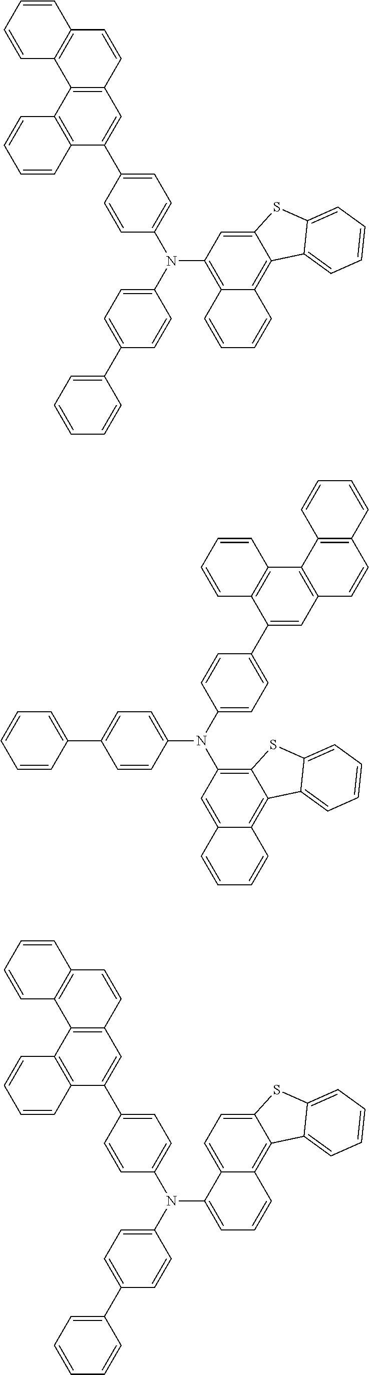 [SMILES: c1ccc(-c2ccc(N(c3ccc(-c4cc5ccc6ccccc6c5c5ccccc45)cc3)c3cc4sc5ccccc5c4c4ccccc34)cc2)cc1 ; c1ccc(-c2ccc(N(c3ccc(-c4cc5ccc6ccccc6c5c5ccccc45)cc3)c3cc4ccccc4c4c3sc3ccccc34)cc2)cc1 ; c1ccc(-c2ccc(N(c3ccc(-c4cc5ccc6ccccc6c5c5ccccc45)cc3)c3cccc4c3ccc3sc5ccccc5c34)cc2)cc1]

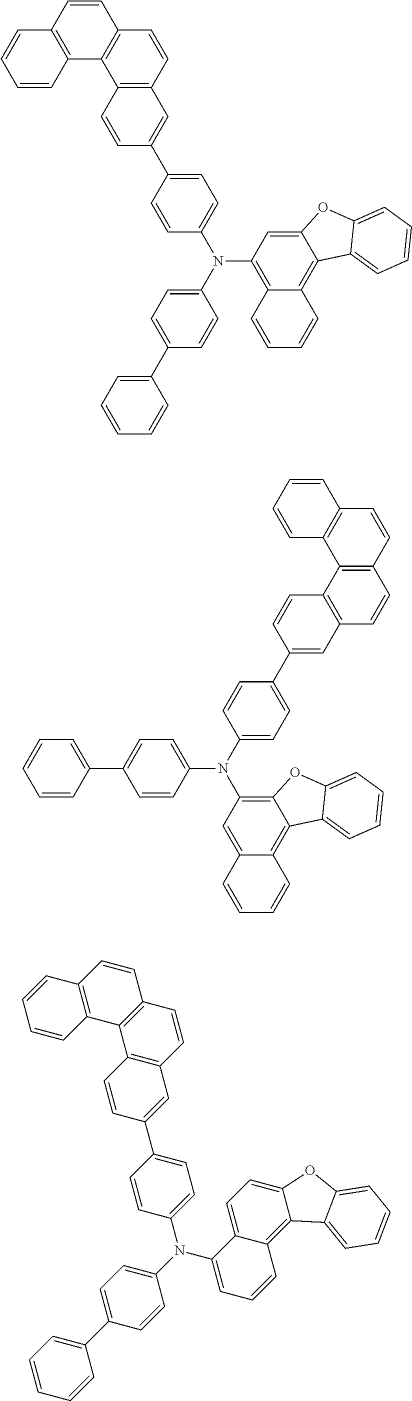 [SMILES: c1ccc(-c2ccc(N(c3ccc(-c4ccc5c(ccc6ccc7ccccc7c65)c4)cc3)c3cc4oc5ccccc5c4c4ccccc34)cc2)cc1 ; c1ccc(-c2ccc(N(c3ccc(-c4ccc5c(ccc6ccc7ccccc7c65)c4)cc3)c3cc4ccccc4c4c3oc3ccccc34)cc2)cc1 ; c1ccc(-c2ccc(N(c3ccc(-c4ccc5c(ccc6ccc7ccccc7c65)c4)cc3)c3cccc4c3ccc3oc5ccccc5c34)cc2)cc1]

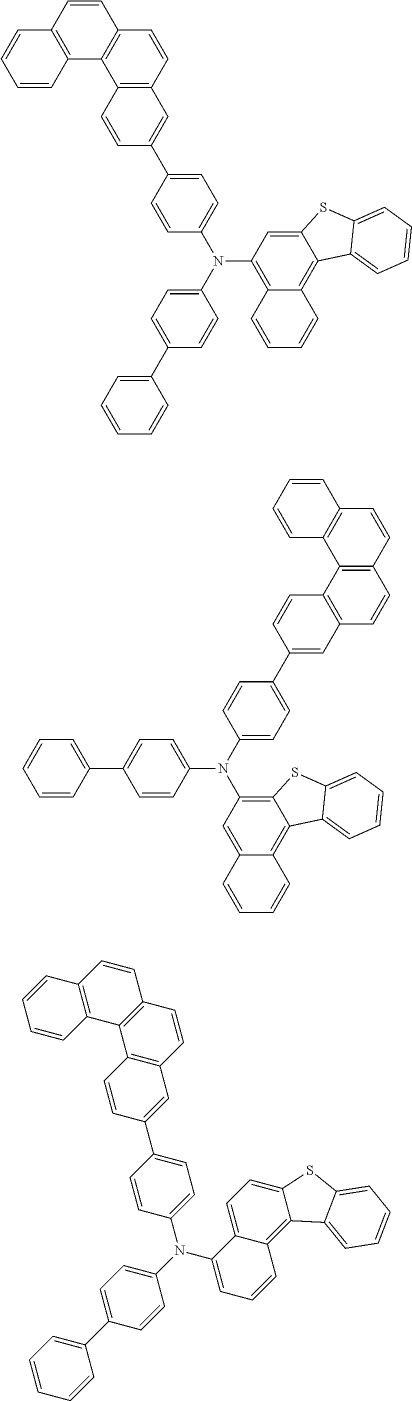 [SMILES: c1ccc(-c2ccc(N(c3ccc(-c4ccc5c(ccc6ccc7ccccc7c65)c4)cc3)c3cc4sc5ccccc5c4c4ccccc34)cc2)cc1 ; c1ccc(-c2ccc(N(c3ccc(-c4ccc5c(ccc6ccc7ccccc7c65)c4)cc3)c3cc4ccccc4c4c3sc3ccccc34)cc2)cc1 ; c1ccc(-c2ccc(N(c3ccc(-c4ccc5c(ccc6ccc7ccccc7c65)c4)cc3)c3cccc4c3ccc3sc5ccccc5c34)cc2)cc1]

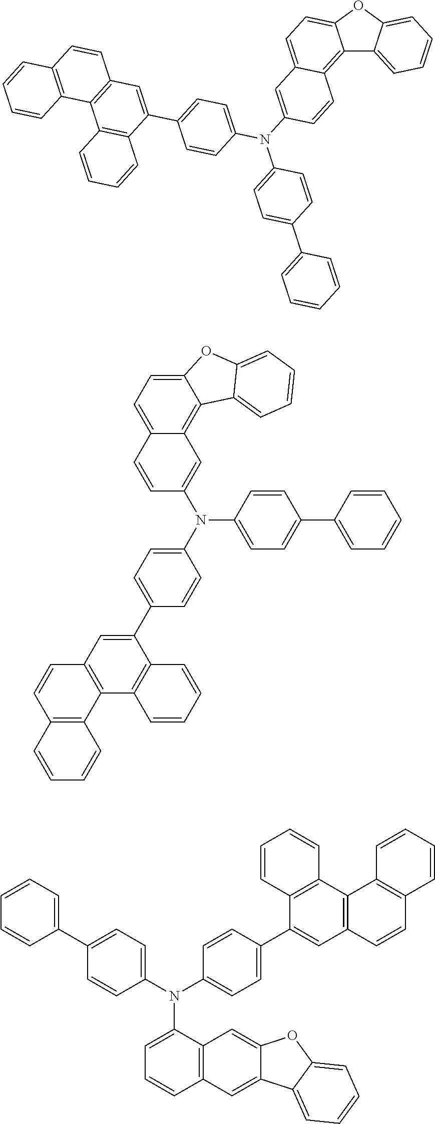 [SMILES: c1ccc(-c2ccc(N(c3ccc(-c4cc5ccc6ccccc6c5c5ccccc45)cc3)c3ccc4c(ccc5oc6ccccc6c54)c3)cc2)cc1 ; c1ccc(-c2ccc(N(c3ccc(-c4cc5ccc6ccccc6c5c5ccccc45)cc3)c3ccc4ccc5oc6ccccc6c5c4c3)cc2)cc1 ; c1ccc(-c2ccc(N(c3ccc(-c4cc5ccc6ccccc6c5c5ccccc45)cc3)c3cccc4cc5c(cc34)oc3ccccc35)cc2)cc1]

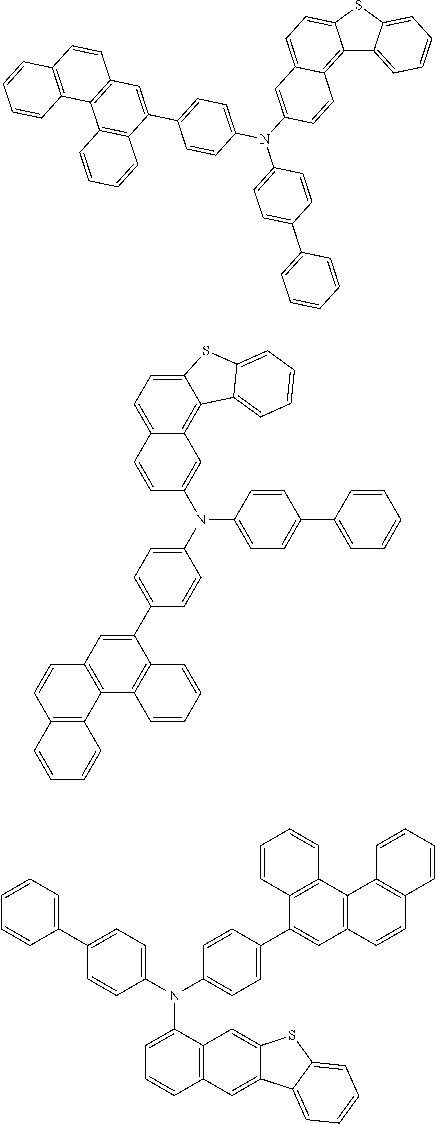 [SMILES: c1ccc(-c2ccc(N(c3ccc(-c4cc5ccc6ccccc6c5c5ccccc45)cc3)c3ccc4c(ccc5sc6ccccc6c54)c3)cc2)cc1 ; c1ccc(-c2ccc(N(c3ccc(-c4cc5ccc6ccccc6c5c5ccccc45)cc3)c3ccc4ccc5sc6ccccc6c5c4c3)cc2)cc1 ; c1ccc(-c2ccc(N(c3ccc(-c4cc5ccc6ccccc6c5c5ccccc45)cc3)c3cccc4cc5c(cc34)sc3ccccc35)cc2)cc1]

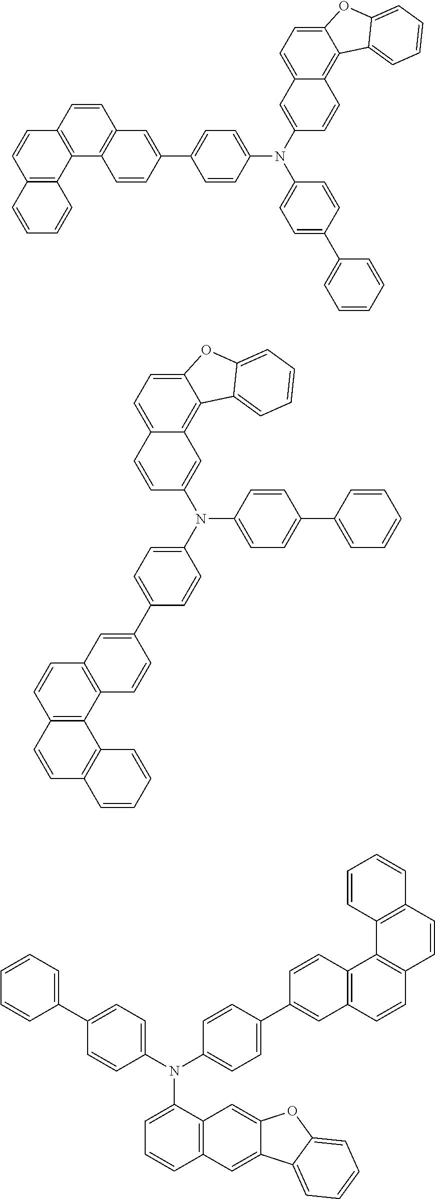 [SMILES: c1ccc(-c2ccc(N(c3ccc(-c4ccc5c(ccc6ccc7ccccc7c65)c4)cc3)c3ccc4c(ccc5oc6ccccc6c54)c3)cc2)cc1 ; c1ccc(-c2ccc(N(c3ccc(-c4ccc5c(ccc6ccc7ccccc7c65)c4)cc3)c3ccc4ccc5oc6ccccc6c5c4c3)cc2)cc1 ; c1ccc(-c2ccc(N(c3ccc(-c4ccc5c(ccc6ccc7ccccc7c65)c4)cc3)c3cccc4cc5c(cc34)oc3ccccc35)cc2)cc1]

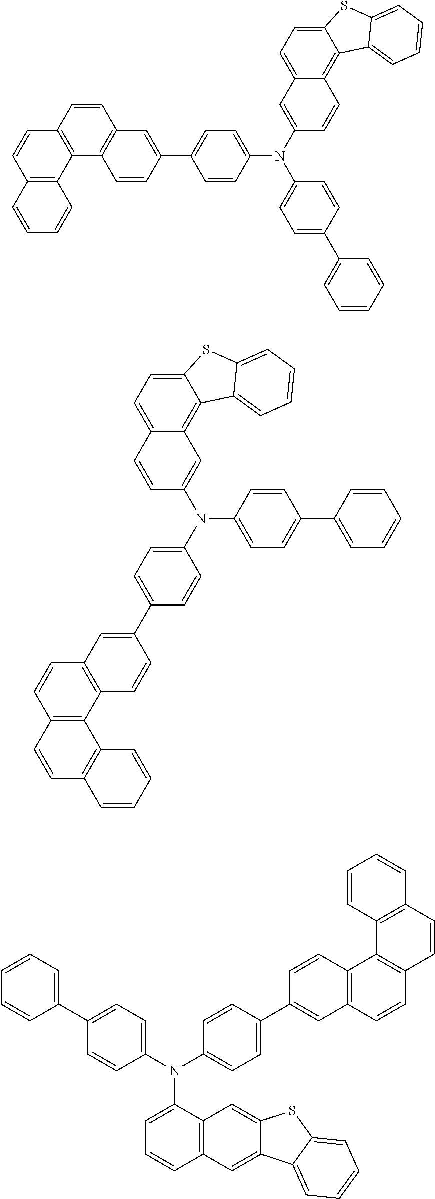 [SMILES: c1ccc(-c2ccc(N(c3ccc(-c4ccc5c(ccc6ccc7ccccc7c65)c4)cc3)c3ccc4c(ccc5sc6ccccc6c54)c3)cc2)cc1 ; c1ccc(-c2ccc(N(c3ccc(-c4ccc5c(ccc6ccc7ccccc7c65)c4)cc3)c3ccc4ccc5sc6ccccc6c5c4c3)cc2)cc1 ; c1ccc(-c2ccc(N(c3ccc(-c4ccc5c(ccc6ccc7ccccc7c65)c4)cc3)c3cccc4cc5c(cc34)sc3ccccc35)cc2)cc1]

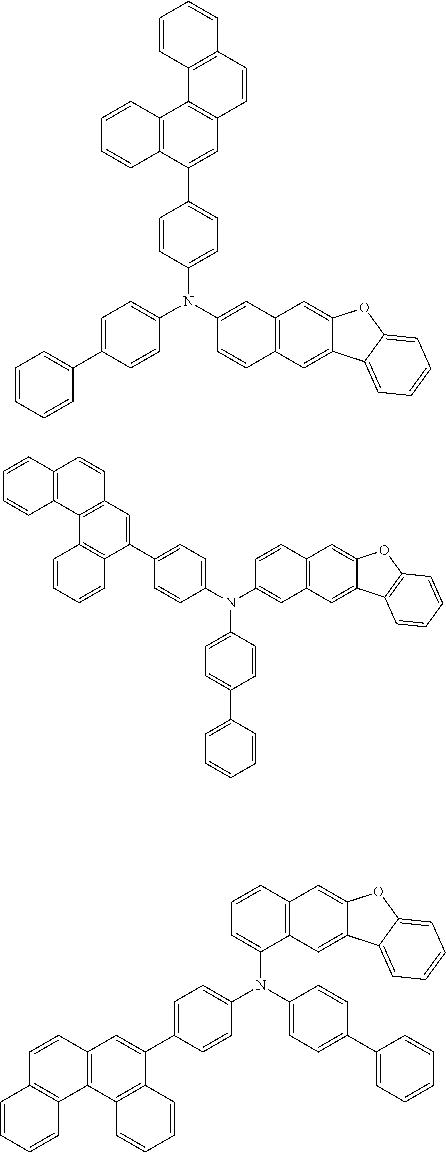

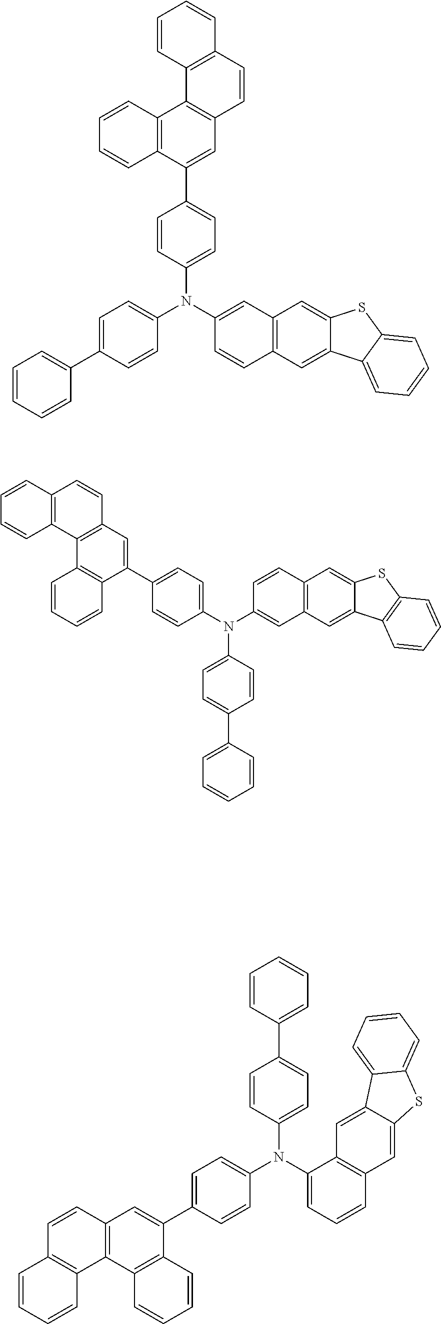 [SMILES: c1ccc(-c2ccc(N(c3ccc(-c4cc5ccc6ccccc6c5c5ccccc45)cc3)c3ccc4cc5c(cc4c3)sc3ccccc35)cc2)cc1 ; c1ccc(-c2ccc(N(c3ccc(-c4cc5ccc6ccccc6c5c5ccccc45)cc3)c3ccc4cc5sc6ccccc6c5cc4c3)cc2)cc1 ; c1ccc(-c2ccc(N(c3ccc(-c4cc5ccc6ccccc6c5c5ccccc45)cc3)c3cccc4cc5sc6ccccc6c5cc34)cc2)cc1]

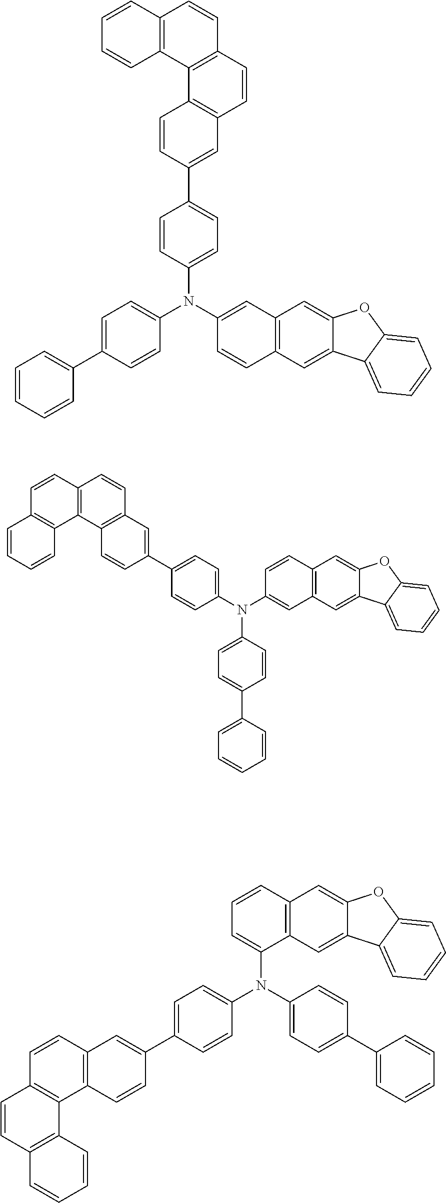 [SMILES: c1ccc(-c2ccc(N(c3ccc(-c4ccc5c(ccc6ccc7ccccc7c65)c4)cc3)c3ccc4cc5c(cc4c3)oc3ccccc35)cc2)cc1 ; c1ccc(-c2ccc(N(c3ccc(-c4ccc5c(ccc6ccc7ccccc7c65)c4)cc3)c3ccc4cc5oc6ccccc6c5cc4c3)cc2)cc1 ; c1ccc(-c2ccc(N(c3ccc(-c4ccc5c(ccc6ccc7ccccc7c65)c4)cc3)c3cccc4cc5oc6ccccc6c5cc34)cc2)cc1]

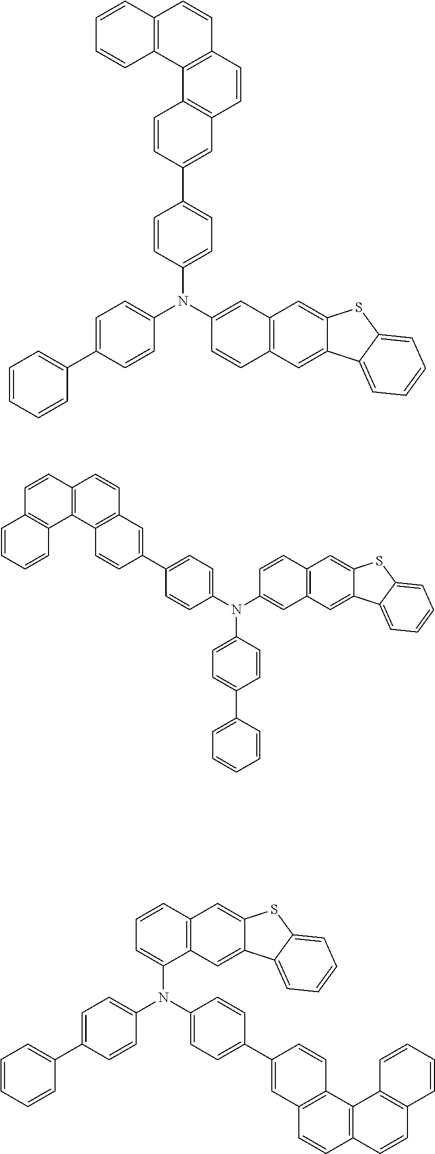 [SMILES: c1ccc(-c2ccc(N(c3ccc(-c4ccc5c(ccc6ccc7ccccc7c65)c4)cc3)c3ccc4cc5c(cc4c3)sc3ccccc35)cc2)cc1 ; c1ccc(-c2ccc(N(c3ccc(-c4ccc5c(ccc6ccc7ccccc7c65)c4)cc3)c3ccc4cc5sc6ccccc6c5cc4c3)cc2)cc1 ; c1ccc(-c2ccc(N(c3ccc(-c4ccc5c(ccc6ccc7ccccc7c65)c4)cc3)c3cccc4cc5sc6ccccc6c5cc34)cc2)cc1]

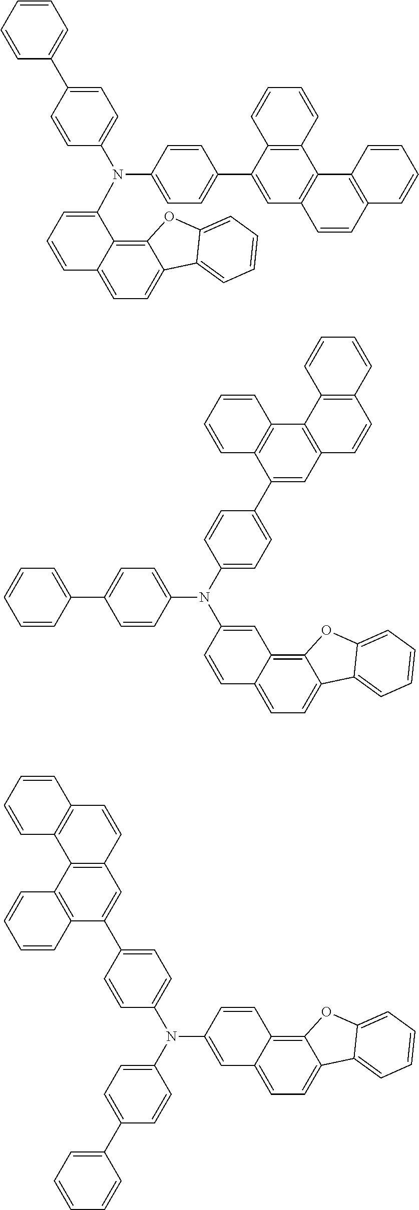 [SMILES: c1ccc(-c2ccc(N(c3ccc(-c4cc5ccc6ccccc6c5c5ccccc45)cc3)c3cccc4ccc5c6ccccc6oc5c34)cc2)cc1 ; c1ccc(-c2ccc(N(c3ccc(-c4cc5ccc6ccccc6c5c5ccccc45)cc3)c3ccc4ccc5c6ccccc6oc5c4c3)cc2)cc1 ; c1ccc(-c2ccc(N(c3ccc(-c4cc5ccc6ccccc6c5c5ccccc45)cc3)c3ccc4c(ccc5c6ccccc6oc45)c3)cc2)cc1]

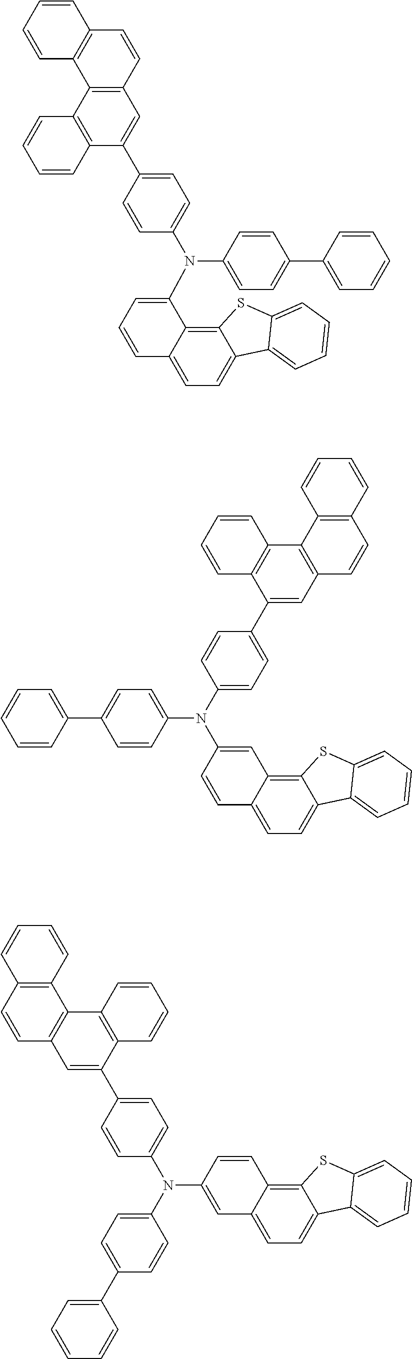 [SMILES: c1ccc(-c2ccc(N(c3ccc(-c4cc5ccc6ccccc6c5c5ccccc45)cc3)c3cccc4ccc5c6ccccc6sc5c34)cc2)cc1 ; c1ccc(-c2ccc(N(c3ccc(-c4cc5ccc6ccccc6c5c5ccccc45)cc3)c3ccc4ccc5c6ccccc6sc5c4c3)cc2)cc1 ; c1ccc(-c2ccc(N(c3ccc(-c4cc5ccc6ccccc6c5c5ccccc45)cc3)c3ccc4c(ccc5c6ccccc6sc45)c3)cc2)cc1]

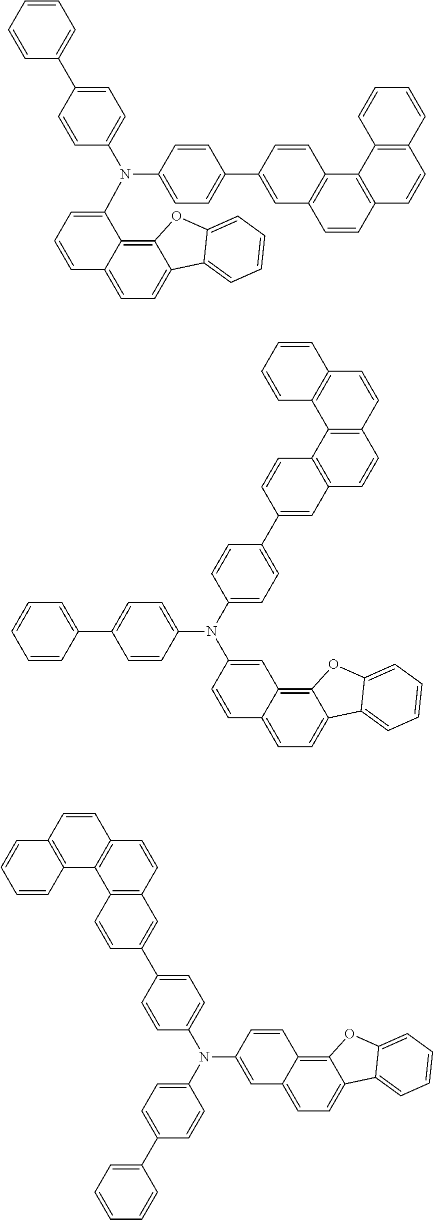 [SMILES: c1ccc(-c2ccc(N(c3ccc(-c4ccc5c(ccc6ccc7ccccc7c65)c4)cc3)c3cccc4ccc5c6ccccc6oc5c34)cc2)cc1 ; c1ccc(-c2ccc(N(c3ccc(-c4ccc5c(ccc6ccc7ccccc7c65)c4)cc3)c3ccc4ccc5c6ccccc6oc5c4c3)cc2)cc1 ; c1ccc(-c2ccc(N(c3ccc(-c4ccc5c(ccc6ccc7ccccc7c65)c4)cc3)c3ccc4c(ccc5c6ccccc6oc45)c3)cc2)cc1]

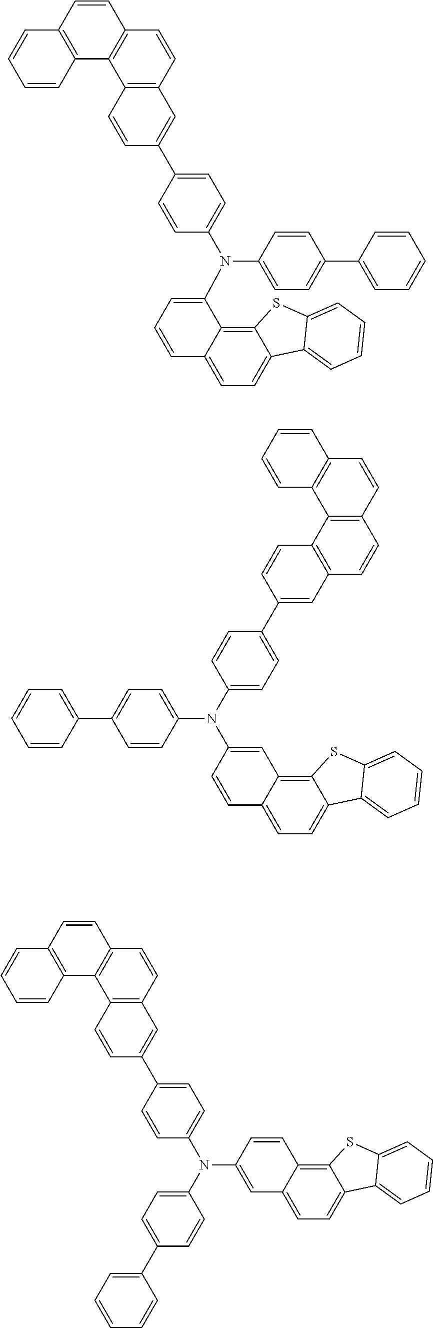 [SMILES: c1ccc(-c2ccc(N(c3ccc(-c4ccc5c(ccc6ccc7ccccc7c65)c4)cc3)c3cccc4ccc5c6ccccc6sc5c34)cc2)cc1 ; c1ccc(-c2ccc(N(c3ccc(-c4ccc5c(ccc6ccc7ccccc7c65)c4)cc3)c3ccc4ccc5c6ccccc6sc5c4c3)cc2)cc1 ; c1ccc(-c2ccc(N(c3ccc(-c4ccc5c(ccc6ccc7ccccc7c65)c4)cc3)c3ccc4c(ccc5c6ccccc6sc45)c3)cc2)cc1]

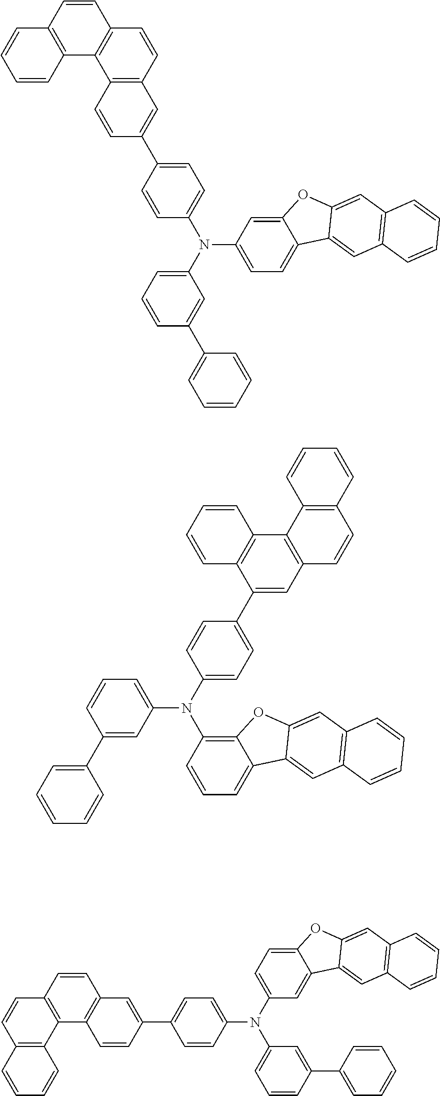 [SMILES: c1ccc(-c2cccc(N(c3ccc(-c4ccc5c(ccc6ccc7ccccc7c65)c4)cc3)c3ccc4c(c3)oc3cc5ccccc5cc34)c2)cc1 ; c1ccc(-c2cccc(N(c3ccc(-c4cc5ccc6ccccc6c5c5ccccc45)cc3)c3cccc4c3oc3cc5ccccc5cc34)c2)cc1 ; c1ccc(-c2cccc(N(c3ccc(-c4ccc5c(ccc6ccc7ccccc7c65)c4)cc3)c3ccc4oc5cc6ccccc6cc5c4c3)c2)cc1]

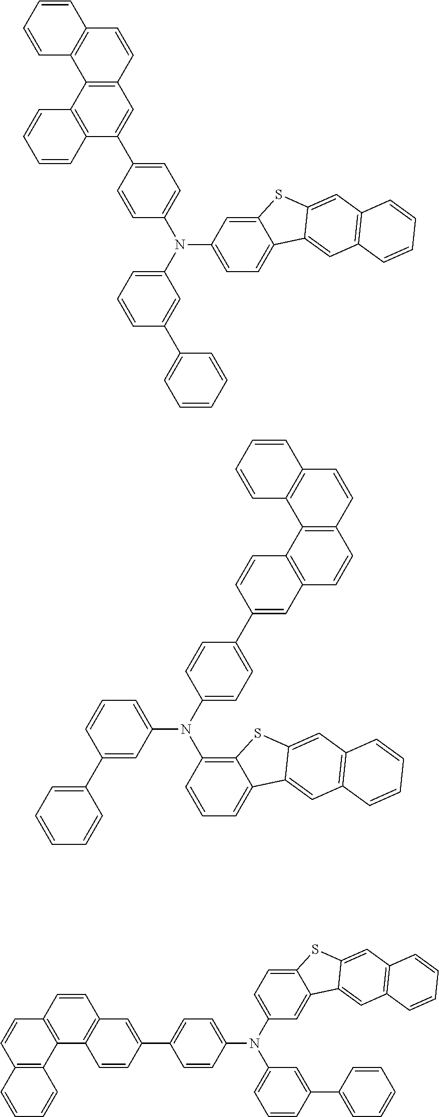 [SMILES: c1ccc(-c2cccc(N(c3ccc(-c4cc5ccc6ccccc6c5c5ccccc45)cc3)c3ccc4c(c3)sc3cc5ccccc5cc34)c2)cc1 ; c1ccc(-c2cccc(N(c3ccc(-c4ccc5c(ccc6ccc7ccccc7c65)c4)cc3)c3cccc4c3sc3cc5ccccc5cc34)c2)cc1 ; c1ccc(-c2cccc(N(c3ccc(-c4ccc5c(ccc6ccc7ccccc7c65)c4)cc3)c3ccc4sc5cc6ccccc6cc5c4c3)c2)cc1]

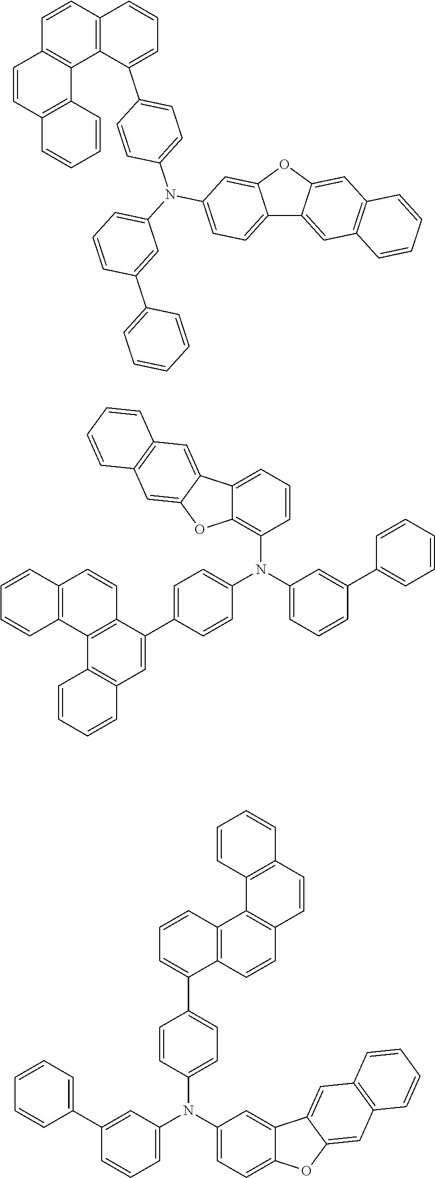 [SMILES: c1ccc(-c2cccc(N(c3ccc(-c4cccc5ccc6ccc7ccccc7c6c45)cc3)c3ccc4c(c3)oc3cc5ccccc5cc34)c2)cc1 ; c1ccc(-c2cccc(N(c3ccc(-c4cc5ccccc5c5c4ccc4ccccc45)cc3)c3cccc4c3oc3cc5ccccc5cc34)c2)cc1 ; c1ccc(-c2cccc(N(c3ccc(-c4cccc5c4ccc4ccc6ccccc6c45)cc3)c3ccc4oc5cc6ccccc6cc5c4c3)c2)cc1]

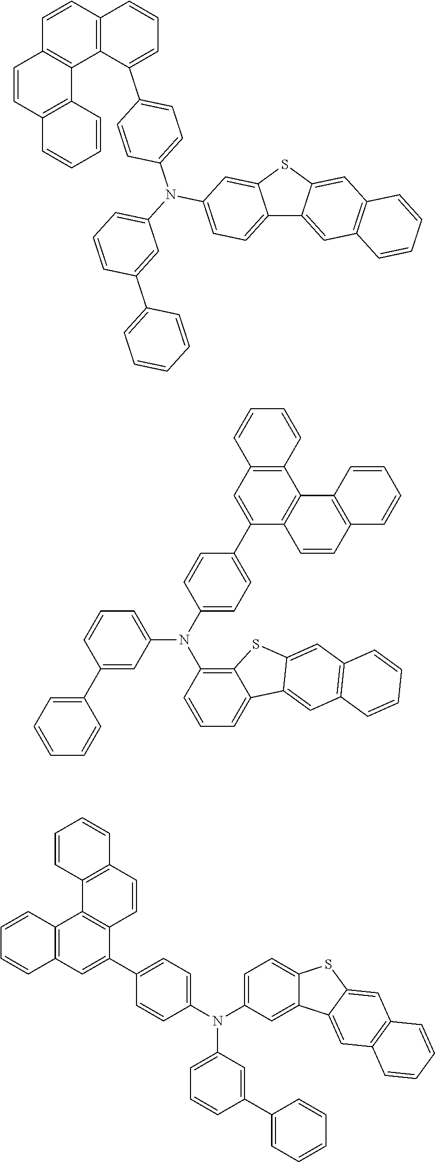 [SMILES: c1ccc(-c2cccc(N(c3ccc(-c4cccc5ccc6ccc7ccccc7c6c45)cc3)c3ccc4c(c3)sc3cc5ccccc5cc34)c2)cc1 ; c1ccc(-c2cccc(N(c3ccc(-c4cc5ccccc5c5c4ccc4ccccc45)cc3)c3cccc4c3sc3cc5ccccc5cc34)c2)cc1 ; c1ccc(-c2cccc(N(c3ccc(-c4cc5ccccc5c5c4ccc4ccccc45)cc3)c3ccc4sc5cc6ccccc6cc5c4c3)c2)cc1]

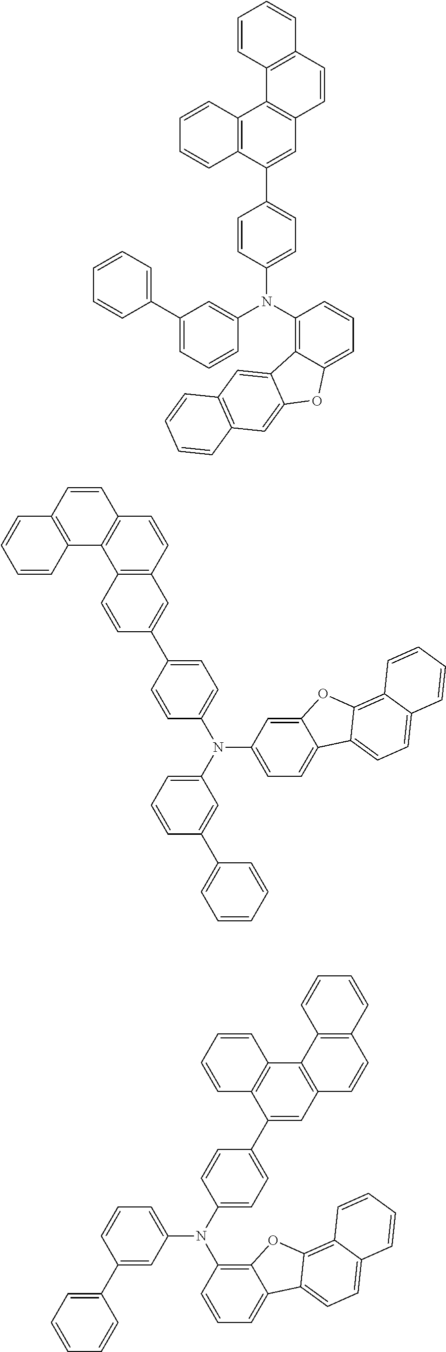 [SMILES: c1ccc(-c2cccc(N(c3ccc(-c4cc5ccc6ccccc6c5c5ccccc45)cc3)c3cccc4oc5cc6ccccc6cc5c34)c2)cc1 ; c1ccc(-c2cccc(N(c3ccc(-c4ccc5c(ccc6ccc7ccccc7c65)c4)cc3)c3ccc4c(c3)oc3c5ccccc5ccc43)c2)cc1 ; c1ccc(-c2cccc(N(c3ccc(-c4cc5ccc6ccccc6c5c5ccccc45)cc3)c3cccc4c3oc3c5ccccc5ccc43)c2)cc1]

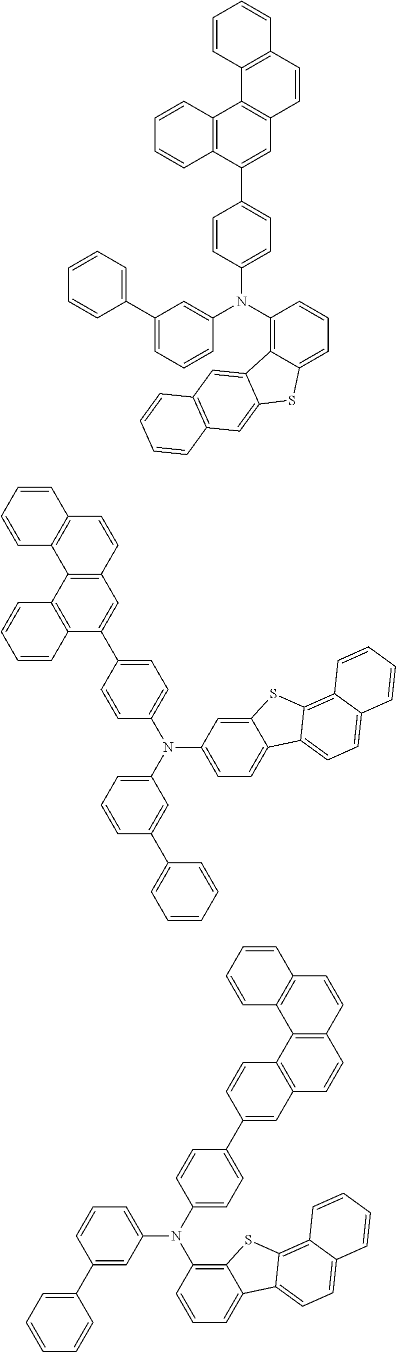 [SMILES: c1ccc(-c2cccc(N(c3ccc(-c4cc5ccc6ccccc6c5c5ccccc45)cc3)c3cccc4sc5cc6ccccc6cc5c34)c2)cc1 ; c1ccc(-c2cccc(N(c3ccc(-c4cc5ccc6ccccc6c5c5ccccc45)cc3)c3ccc4c(c3)sc3c5ccccc5ccc43)c2)cc1 ; c1ccc(-c2cccc(N(c3ccc(-c4ccc5c(ccc6ccc7ccccc7c65)c4)cc3)c3cccc4c3sc3c5ccccc5ccc43)c2)cc1]

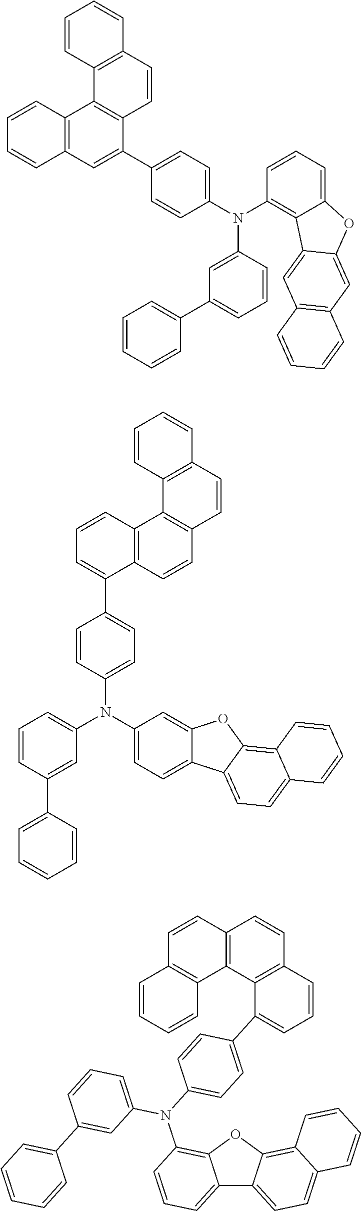

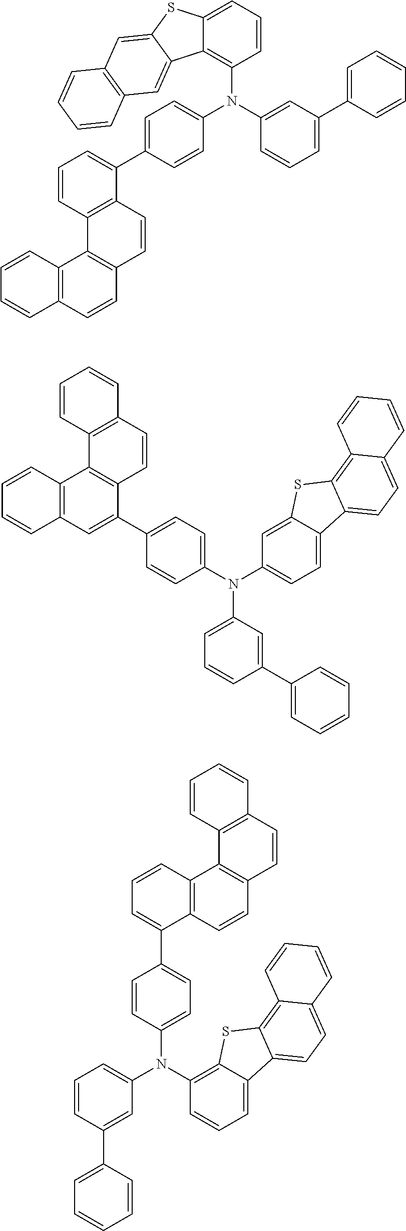 [SMILES: c1ccc(-c2cccc(N(c3ccc(-c4cccc5c4ccc4ccc6ccccc6c45)cc3)c3cccc4sc5cc6ccccc6cc5c34)c2)cc1 ; c1ccc(-c2cccc(N(c3ccc(-c4cc5ccccc5c5c4ccc4ccccc45)cc3)c3ccc4c(c3)sc3c5ccccc5ccc43)c2)cc1 ; c1ccc(-c2cccc(N(c3ccc(-c4cccc5c4ccc4ccc6ccccc6c45)cc3)c3cccc4c3sc3c5ccccc5ccc43)c2)cc1]

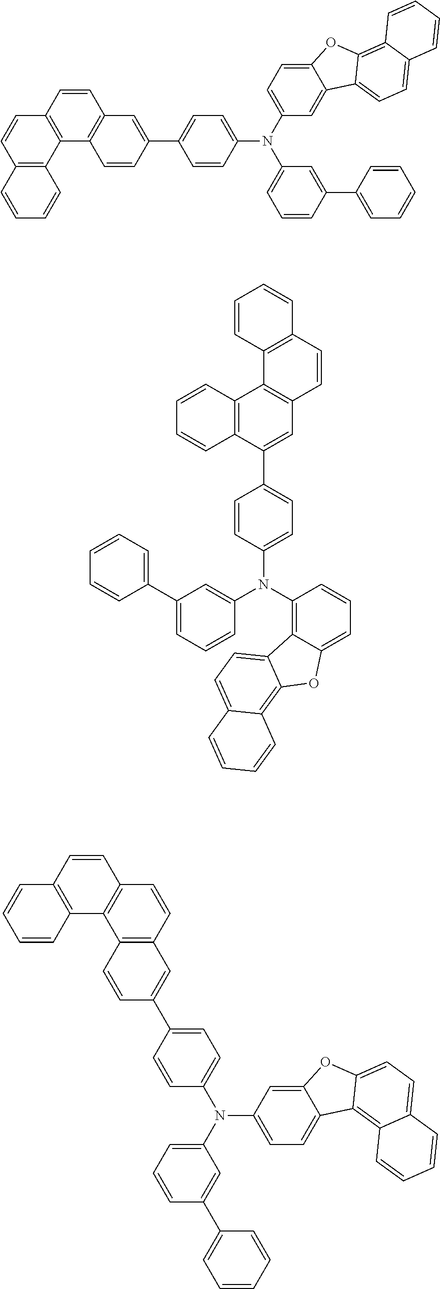 [SMILES: c1ccc(-c2cccc(N(c3ccc(-c4ccc5c(ccc6ccc7ccccc7c65)c4)cc3)c3ccc4oc5c6ccccc6ccc5c4c3)c2)cc1 ; c1ccc(-c2cccc(N(c3ccc(-c4cc5ccc6ccccc6c5c5ccccc45)cc3)c3cccc4oc5c6ccccc6ccc5c34)c2)cc1 ; c1ccc(-c2cccc(N(c3ccc(-c4ccc5c(ccc6ccc7ccccc7c65)c4)cc3)c3ccc4c(c3)oc3ccc5ccccc5c34)c2)cc1]

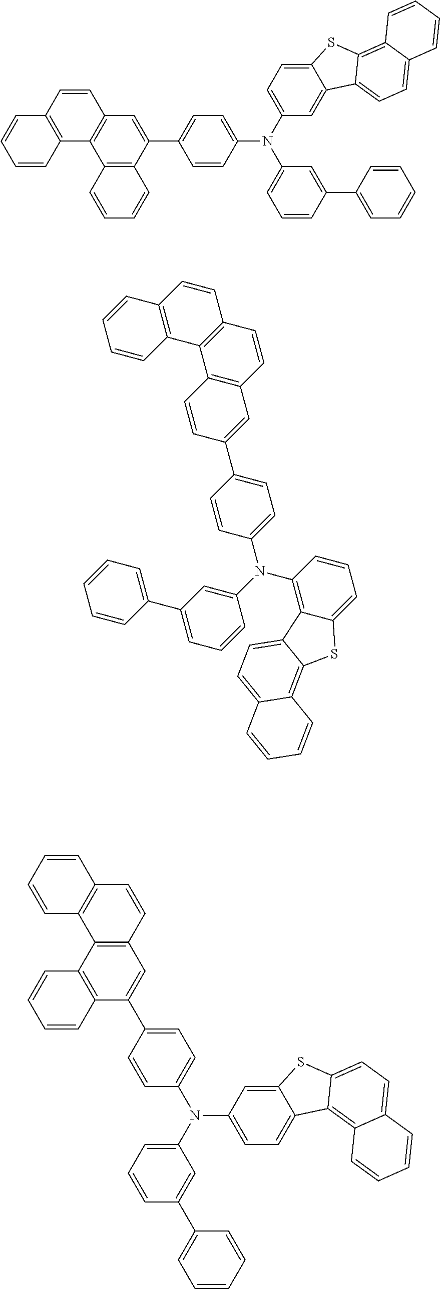 [SMILES: c1ccc(-c2cccc(N(c3ccc(-c4cc5ccc6ccccc6c5c5ccccc45)cc3)c3ccc4sc5c6ccccc6ccc5c4c3)c2)cc1 ; c1ccc(-c2cccc(N(c3ccc(-c4ccc5c(ccc6ccc7ccccc7c65)c4)cc3)c3cccc4sc5c6ccccc6ccc5c34)c2)cc1 ; c1ccc(-c2cccc(N(c3ccc(-c4cc5ccc6ccccc6c5c5ccccc45)cc3)c3ccc4c(c3)sc3ccc5ccccc5c34)c2)cc1]

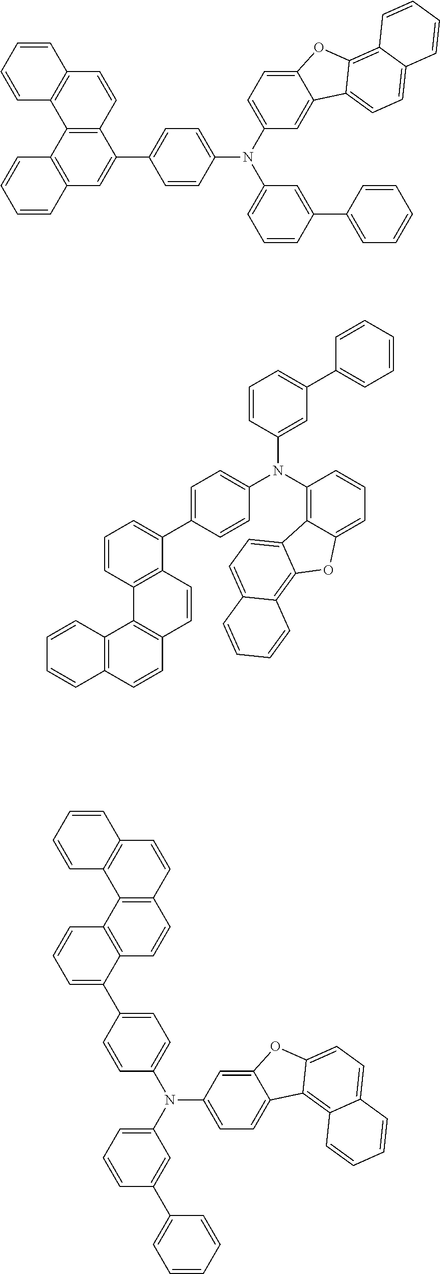 [SMILES: c1ccc(-c2cccc(N(c3ccc(-c4cc5ccccc5c5c4ccc4ccccc45)cc3)c3ccc4oc5c6ccccc6ccc5c4c3)c2)cc1 ; c1ccc(-c2cccc(N(c3ccc(-c4cccc5c4ccc4ccc6ccccc6c45)cc3)c3cccc4oc5c6ccccc6ccc5c34)c2)cc1 ; c1ccc(-c2cccc(N(c3ccc(-c4cccc5c4ccc4ccc6ccccc6c45)cc3)c3ccc4c(c3)oc3ccc5ccccc5c34)c2)cc1]

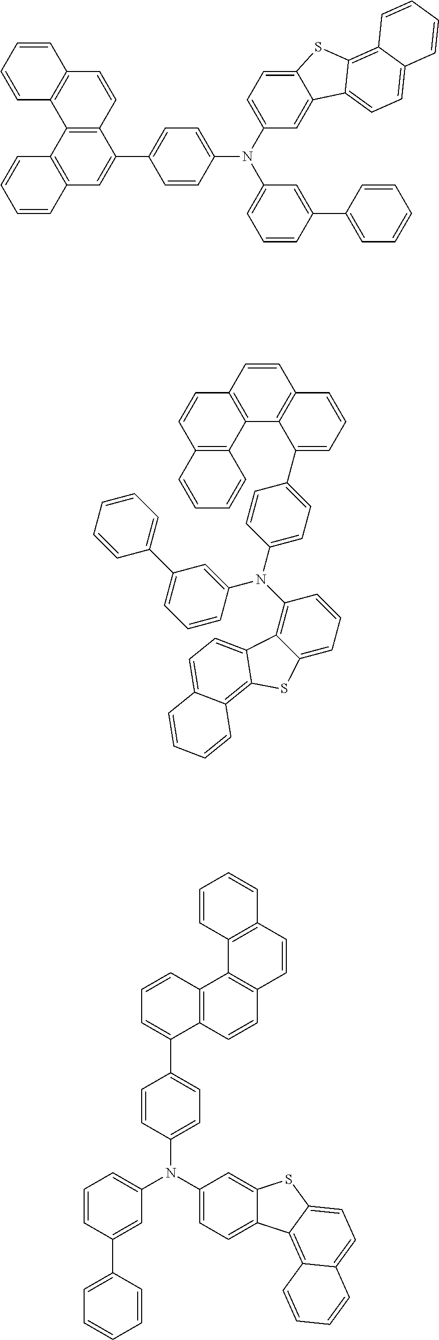 [SMILES: c1ccc(-c2cccc(N(c3ccc(-c4cc5ccccc5c5c4ccc4ccccc45)cc3)c3ccc4sc5c6ccccc6ccc5c4c3)c2)cc1 ; c1ccc(-c2cccc(N(c3ccc(-c4cccc5ccc6ccc7ccccc7c6c45)cc3)c3cccc4sc5c6ccccc6ccc5c34)c2)cc1 ; c1ccc(-c2cccc(N(c3ccc(-c4cccc5c4ccc4ccc6ccccc6c45)cc3)c3ccc4c(c3)sc3ccc5ccccc5c34)c2)cc1]

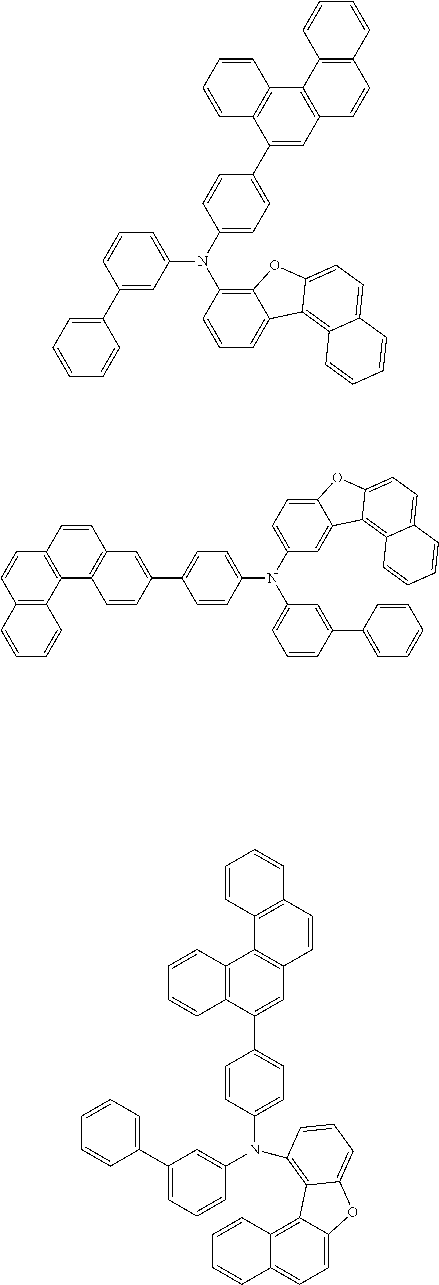 [SMILES: c1ccc(-c2cccc(N(c3ccc(-c4cc5ccc6ccccc6c5c5ccccc45)cc3)c3cccc4c3oc3ccc5ccccc5c34)c2)cc1 ; c1ccc(-c2cccc(N(c3ccc(-c4ccc5c(ccc6ccc7ccccc7c65)c4)cc3)c3ccc4oc5ccc6ccccc6c5c4c3)c2)cc1 ; c1ccc(-c2cccc(N(c3ccc(-c4cc5ccc6ccccc6c5c5ccccc45)cc3)c3cccc4oc5ccc6ccccc6c5c34)c2)cc1]

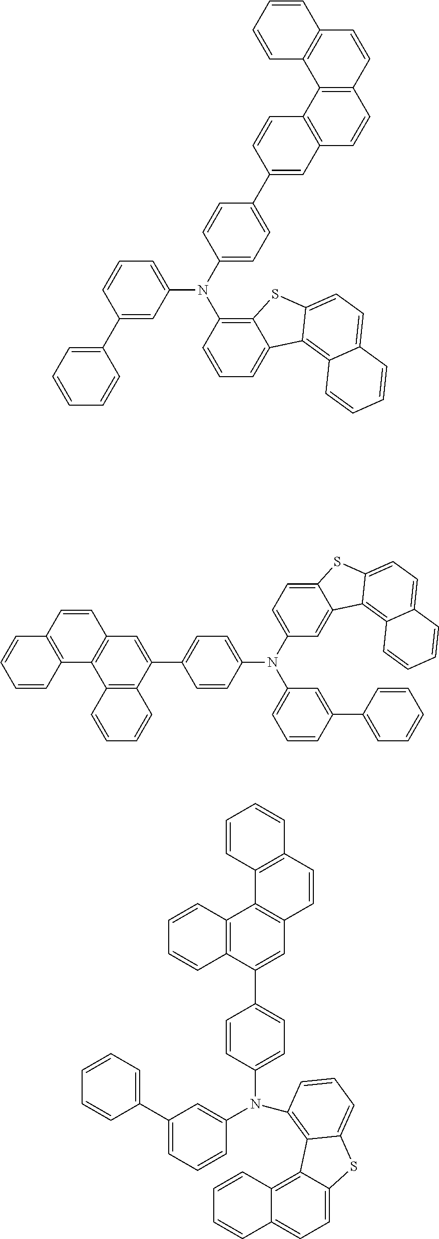 [SMILES: c1ccc(-c2cccc(N(c3ccc(-c4ccc5c(ccc6ccc7ccccc7c65)c4)cc3)c3cccc4c3sc3ccc5ccccc5c34)c2)cc1 ; c1ccc(-c2cccc(N(c3ccc(-c4cc5ccc6ccccc6c5c5ccccc45)cc3)c3ccc4sc5ccc6ccccc6c5c4c3)c2)cc1 ; c1ccc(-c2cccc(N(c3ccc(-c4cc5ccc6ccccc6c5c5ccccc45)cc3)c3cccc4sc5ccc6ccccc6c5c34)c2)cc1]

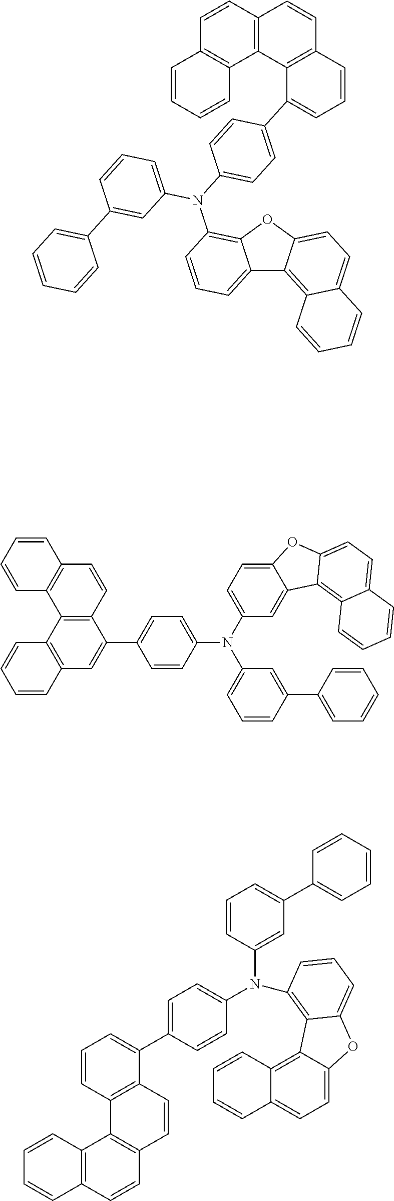 [SMILES: c1ccc(-c2cccc(N(c3ccc(-c4cccc5ccc6ccc7ccccc7c6c45)cc3)c3cccc4c3oc3ccc5ccccc5c34)c2)cc1 ; c1ccc(-c2cccc(N(c3ccc(-c4cc5ccccc5c5c4ccc4ccccc45)cc3)c3ccc4oc5ccc6ccccc6c5c4c3)c2)cc1 ; c1ccc(-c2cccc(N(c3ccc(-c4cccc5c4ccc4ccc6ccccc6c45)cc3)c3cccc4oc5ccc6ccccc6c5c34)c2)cc1]

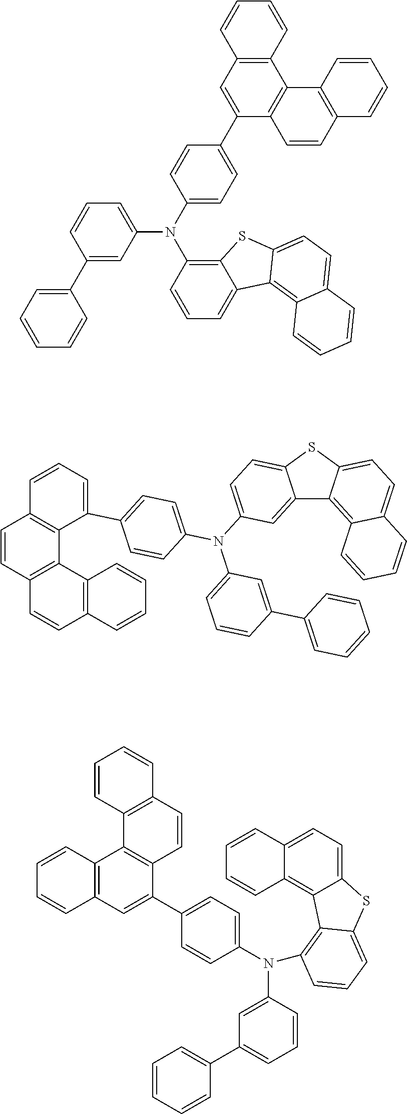 [SMILES: c1ccc(-c2cccc(N(c3ccc(-c4cc5ccccc5c5c4ccc4ccccc45)cc3)c3cccc4c3sc3ccc5ccccc5c34)c2)cc1 ; c1ccc(-c2cccc(N(c3ccc(-c4cccc5ccc6ccc7ccccc7c6c45)cc3)c3ccc4sc5ccc6ccccc6c5c4c3)c2)cc1 ; c1ccc(-c2cccc(N(c3ccc(-c4cc5ccccc5c5c4ccc4ccccc45)cc3)c3cccc4sc5ccc6ccccc6c5c34)c2)cc1]

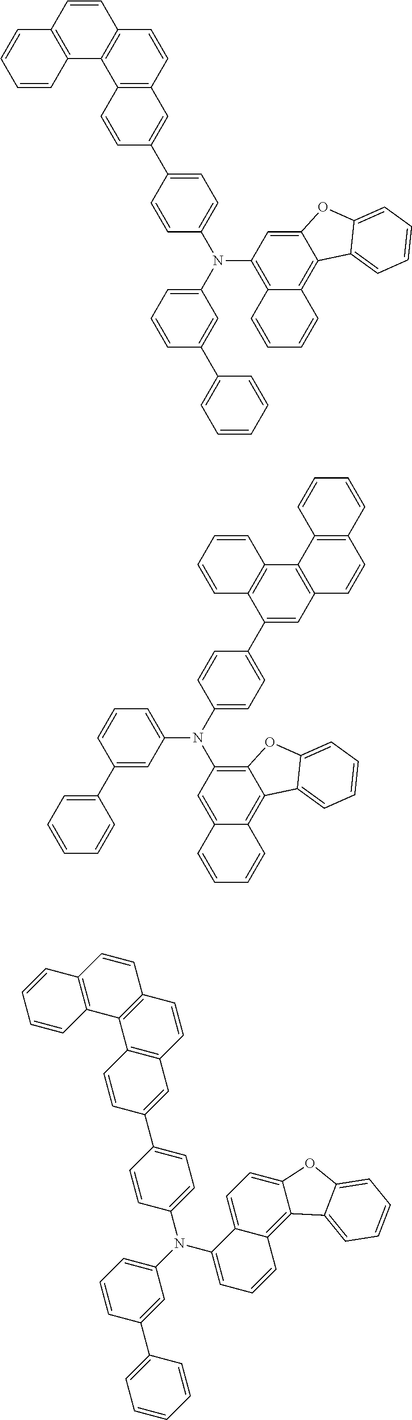 [SMILES: c1ccc(-c2cccc(N(c3ccc(-c4ccc5c(ccc6ccc7ccccc7c65)c4)cc3)c3cc4oc5ccccc5c4c4ccccc34)c2)cc1 ; c1ccc(-c2cccc(N(c3ccc(-c4cc5ccc6ccccc6c5c5ccccc45)cc3)c3cc4ccccc4c4c3oc3ccccc34)c2)cc1 ; c1ccc(-c2cccc(N(c3ccc(-c4ccc5c(ccc6ccc7ccccc7c65)c4)cc3)c3cccc4c3ccc3oc5ccccc5c34)c2)cc1]

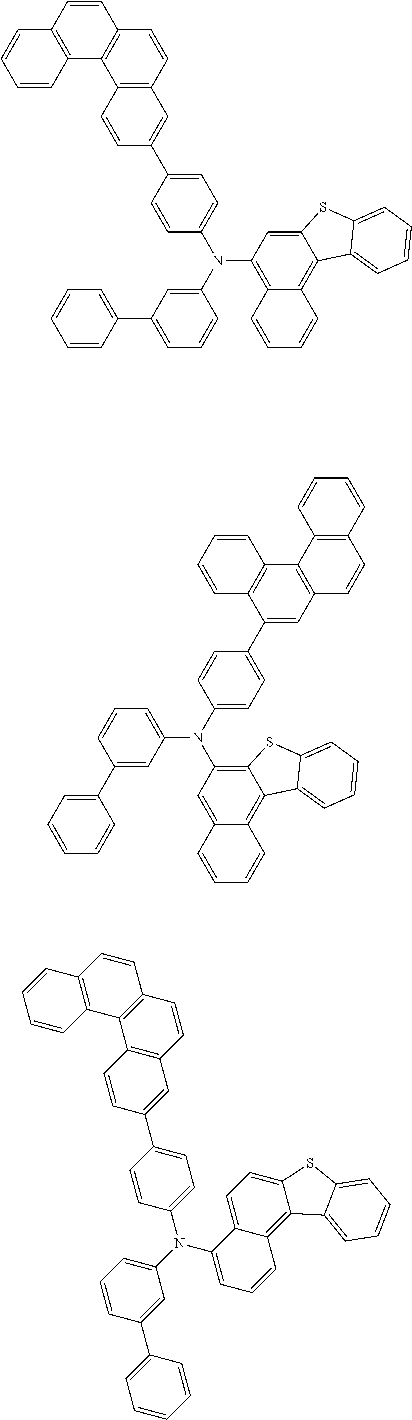 [SMILES: c1ccc(-c2cccc(N(c3ccc(-c4ccc5c(ccc6ccc7ccccc7c65)c4)cc3)c3cc4sc5ccccc5c4c4ccccc34)c2)cc1 ; c1ccc(-c2cccc(N(c3ccc(-c4cc5ccc6ccccc6c5c5ccccc45)cc3)c3cc4ccccc4c4c3sc3ccccc34)c2)cc1 ; c1ccc(-c2cccc(N(c3ccc(-c4ccc5c(ccc6ccc7ccccc7c65)c4)cc3)c3cccc4c3ccc3sc5ccccc5c34)c2)cc1]

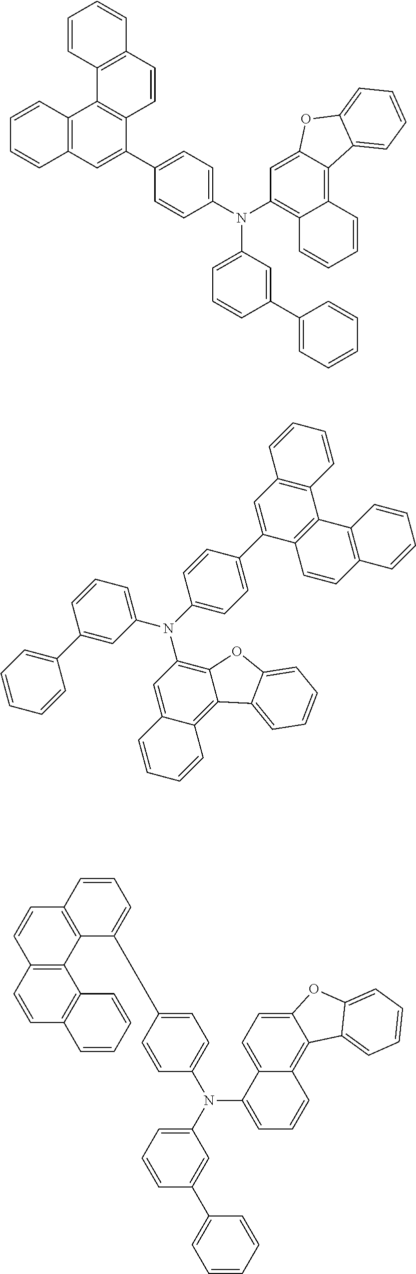 [SMILES: c1ccc(-c2cccc(N(c3ccc(-c4cc5ccccc5c5c4ccc4ccccc45)cc3)c3cc4oc5ccccc5c4c4ccccc34)c2)cc1 ; c1ccc(-c2cccc(N(c3ccc(-c4cc5ccccc5c5c4ccc4ccccc45)cc3)c3cc4ccccc4c4c3oc3ccccc34)c2)cc1 ; c1ccc(-c2cccc(N(c3ccc(-c4cccc5ccc6ccc7ccccc7c6c45)cc3)c3cccc4c3ccc3oc5ccccc5c34)c2)cc1]

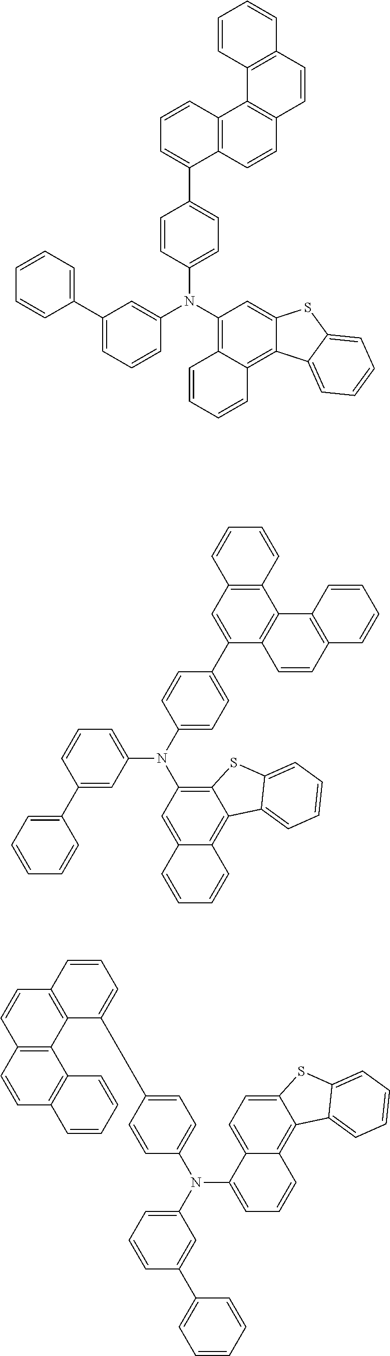 [SMILES: c1ccc(-c2cccc(N(c3ccc(-c4cccc5c4ccc4ccc6ccccc6c45)cc3)c3cc4sc5ccccc5c4c4ccccc34)c2)cc1 ; c1ccc(-c2cccc(N(c3ccc(-c4cc5ccccc5c5c4ccc4ccccc45)cc3)c3cc4ccccc4c4c3sc3ccccc34)c2)cc1 ; c1ccc(-c2cccc(N(c3ccc(-c4cccc5ccc6ccc7ccccc7c6c45)cc3)c3cccc4c3ccc3sc5ccccc5c34)c2)cc1]

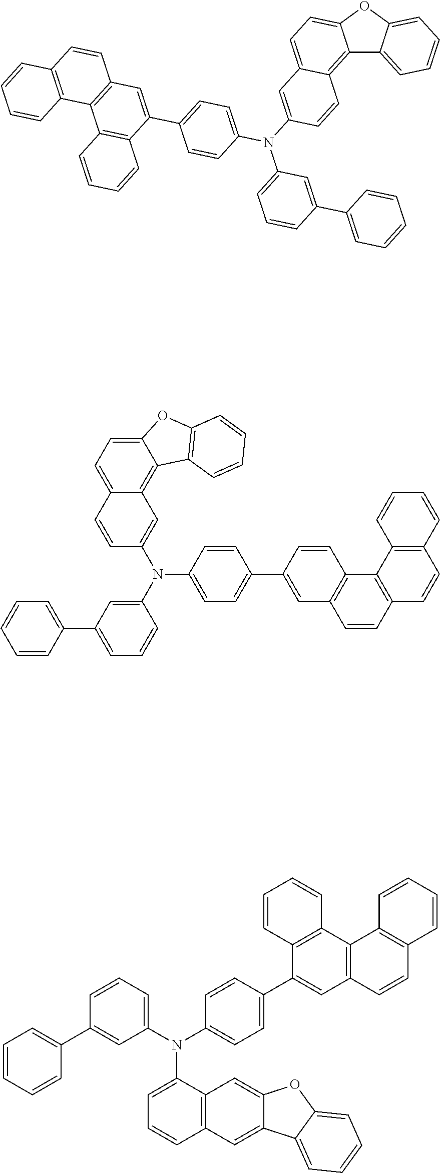 [SMILES: c1ccc(-c2cccc(N(c3ccc(-c4cc5ccc6ccccc6c5c5ccccc45)cc3)c3ccc4c(ccc5oc6ccccc6c54)c3)c2)cc1 ; c1ccc(-c2cccc(N(c3ccc(-c4ccc5c(ccc6ccc7ccccc7c65)c4)cc3)c3ccc4ccc5oc6ccccc6c5c4c3)c2)cc1 ; c1ccc(-c2cccc(N(c3ccc(-c4cc5ccc6ccccc6c5c5ccccc45)cc3)c3cccc4cc5c(cc34)oc3ccccc35)c2)cc1]

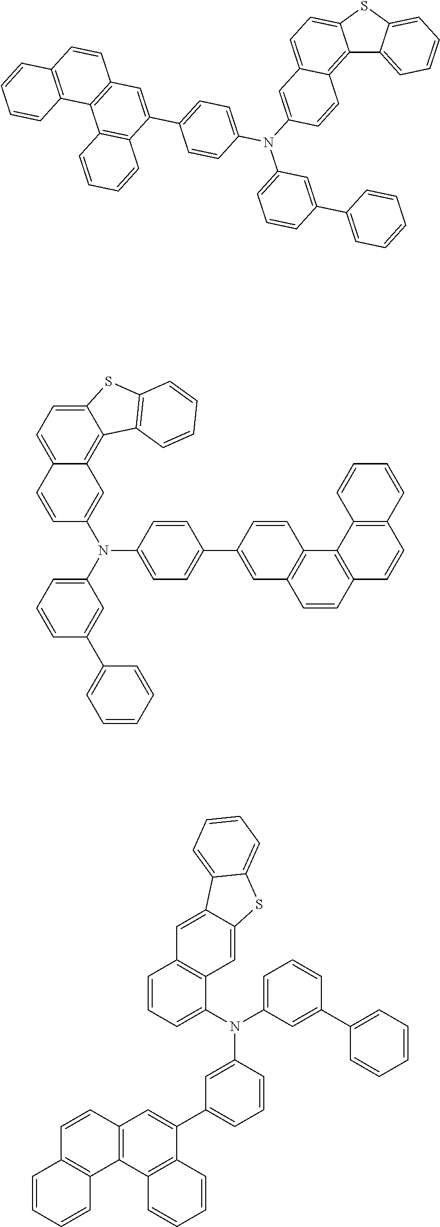 [SMILES: c1ccc(-c2cccc(N(c3ccc(-c4cc5ccc6ccccc6c5c5ccccc45)cc3)c3ccc4c(ccc5sc6ccccc6c54)c3)c2)cc1 ; c1ccc(-c2cccc(N(c3ccc(-c4ccc5c(ccc6ccc7ccccc7c65)c4)cc3)c3ccc4ccc5sc6ccccc6c5c4c3)c2)cc1 ; c1ccc(-c2cccc(N(c3cccc(-c4cc5ccc6ccccc6c5c5ccccc45)c3)c3cccc4cc5c(cc34)sc3ccccc35)c2)cc1]

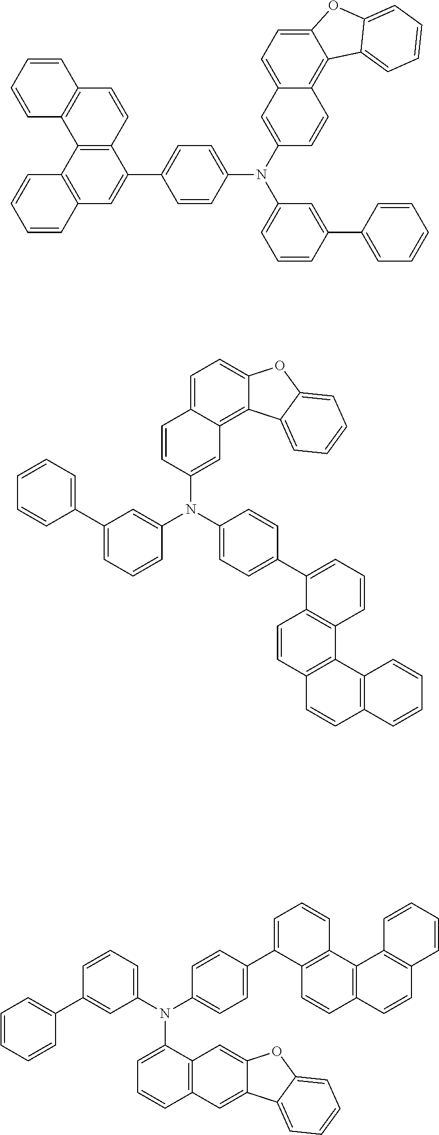 [SMILES: c1ccc(-c2cccc(N(c3ccc(-c4cc5ccccc5c5c4ccc4ccccc45)cc3)c3ccc4c(ccc5oc6ccccc6c54)c3)c2)cc1 ; c1ccc(-c2cccc(N(c3ccc(-c4cccc5c4ccc4ccc6ccccc6c45)cc3)c3ccc4ccc5oc6ccccc6c5c4c3)c2)cc1 ; c1ccc(-c2cccc(N(c3ccc(-c4cccc5c4ccc4ccc6ccccc6c45)cc3)c3cccc4cc5c(cc34)oc3ccccc35)c2)cc1]

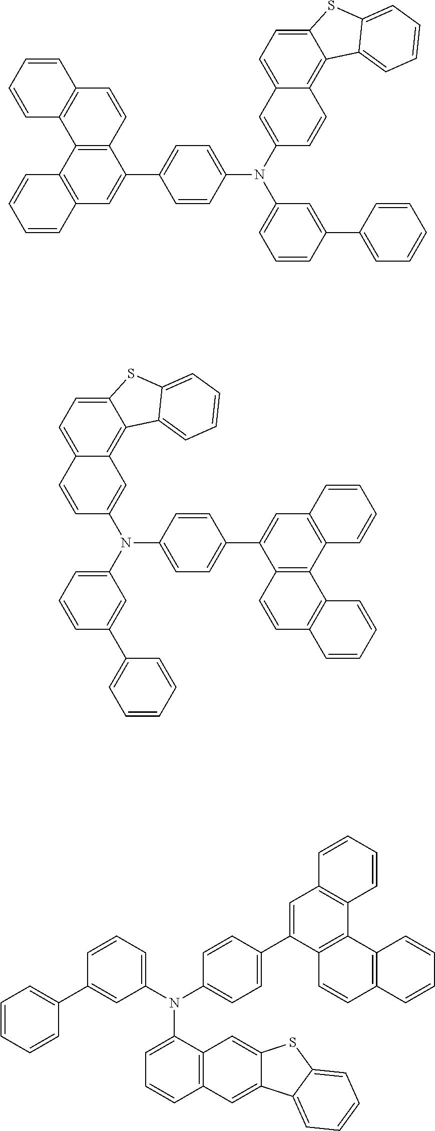 [SMILES: c1ccc(-c2cccc(N(c3ccc(-c4cc5ccccc5c5c4ccc4ccccc45)cc3)c3ccc4c(ccc5sc6ccccc6c54)c3)c2)cc1 ; c1ccc(-c2cccc(N(c3ccc(-c4cc5ccccc5c5c4ccc4ccccc45)cc3)c3ccc4ccc5sc6ccccc6c5c4c3)c2)cc1 ; c1ccc(-c2cccc(N(c3ccc(-c4cc5ccccc5c5c4ccc4ccccc45)cc3)c3cccc4cc5c(cc34)sc3ccccc35)c2)cc1]

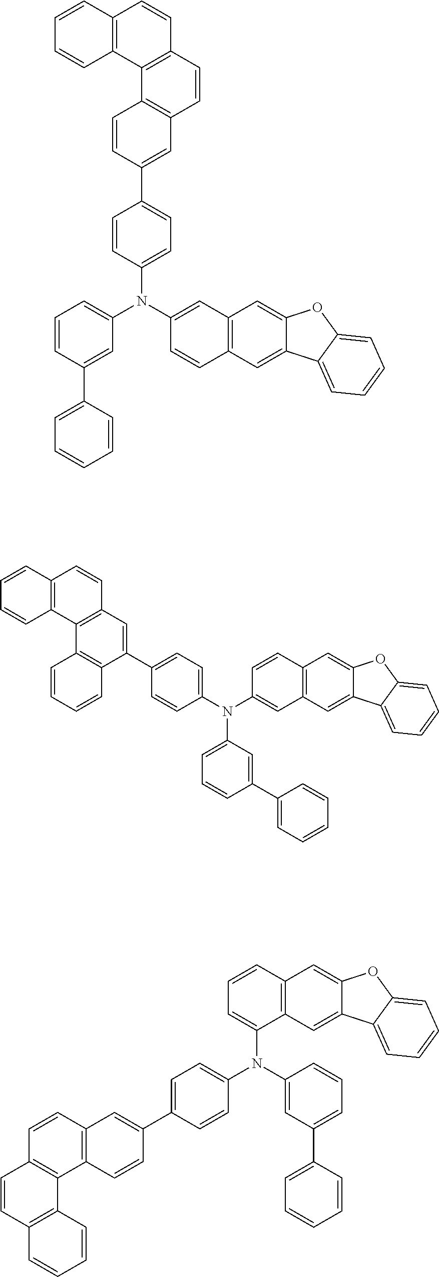 [SMILES: c1ccc(-c2cccc(N(c3ccc(-c4ccc5c(ccc6ccc7ccccc7c65)c4)cc3)c3ccc4cc5c(cc4c3)oc3ccccc35)c2)cc1 ; c1ccc(-c2cccc(N(c3ccc(-c4cc5ccc6ccccc6c5c5ccccc45)cc3)c3ccc4cc5oc6ccccc6c5cc4c3)c2)cc1 ; c1ccc(-c2cccc(N(c3ccc(-c4ccc5c(ccc6ccc7ccccc7c65)c4)cc3)c3cccc4cc5oc6ccccc6c5cc34)c2)cc1]

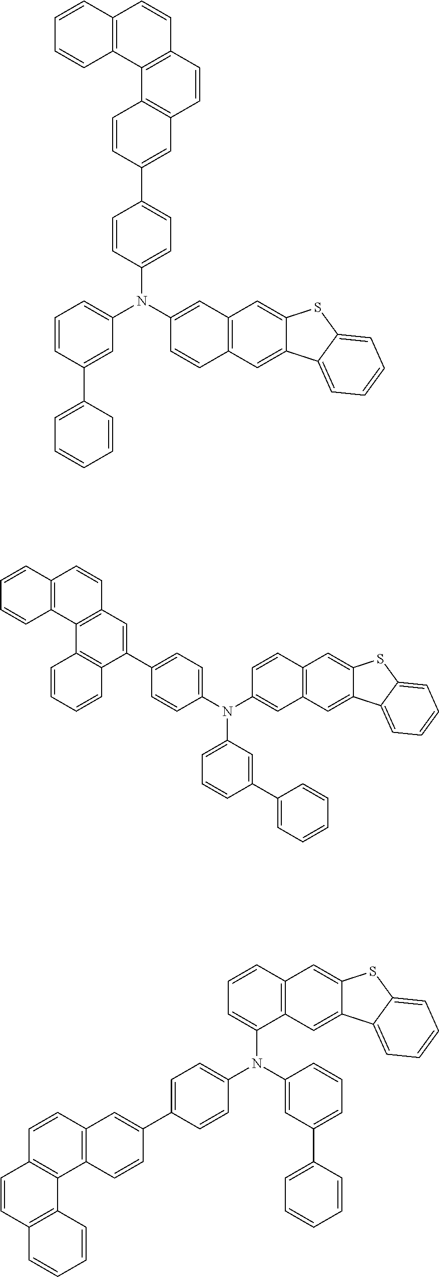 [SMILES: c1ccc(-c2cccc(N(c3ccc(-c4ccc5c(ccc6ccc7ccccc7c65)c4)cc3)c3ccc4cc5c(cc4c3)sc3ccccc35)c2)cc1 ; c1ccc(-c2cccc(N(c3ccc(-c4cc5ccc6ccccc6c5c5ccccc45)cc3)c3ccc4cc5sc6ccccc6c5cc4c3)c2)cc1 ; c1ccc(-c2cccc(N(c3ccc(-c4ccc5c(ccc6ccc7ccccc7c65)c4)cc3)c3cccc4cc5sc6ccccc6c5cc34)c2)cc1]

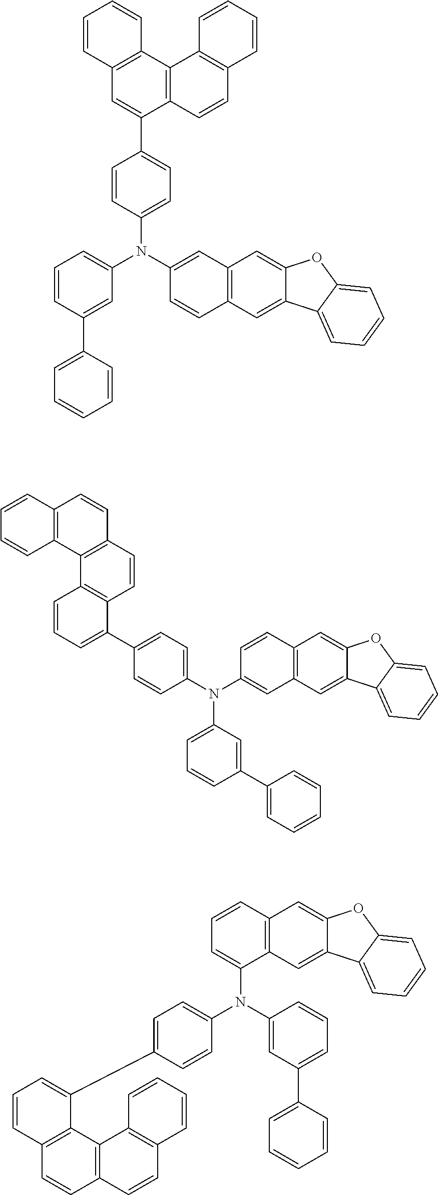 [SMILES: c1ccc(-c2cccc(N(c3ccc(-c4cc5ccccc5c5c4ccc4ccccc45)cc3)c3ccc4cc5c(cc4c3)oc3ccccc35)c2)cc1 ; c1ccc(-c2cccc(N(c3ccc(-c4cccc5c4ccc4ccc6ccccc6c45)cc3)c3ccc4cc5oc6ccccc6c5cc4c3)c2)cc1 ; c1ccc(-c2cccc(N(c3ccc(-c4cccc5ccc6ccc7ccccc7c6c45)cc3)c3cccc4cc5oc6ccccc6c5cc34)c2)cc1]

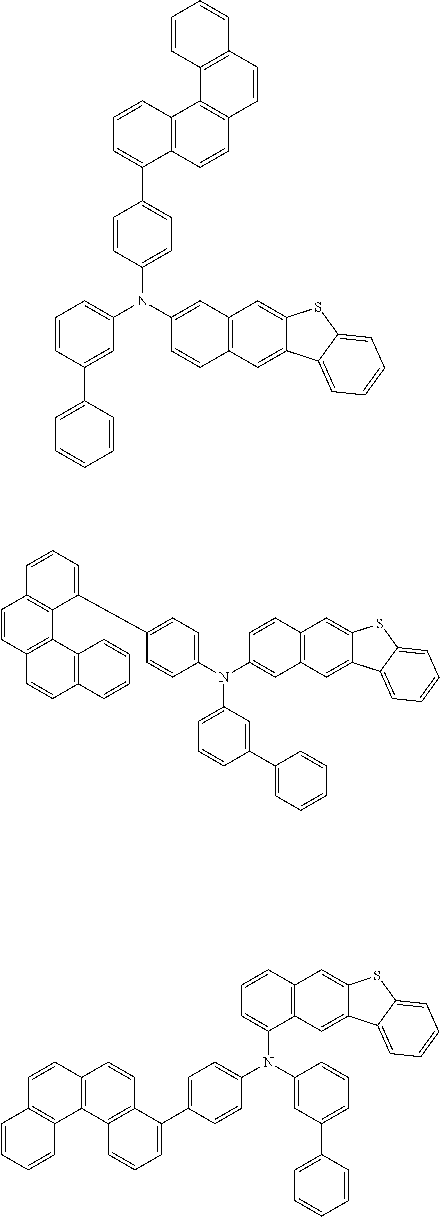 [SMILES: c1ccc(-c2cccc(N(c3ccc(-c4cccc5c4ccc4ccc6ccccc6c45)cc3)c3ccc4cc5c(cc4c3)sc3ccccc35)c2)cc1 ; c1ccc(-c2cccc(N(c3ccc(-c4cccc5ccc6ccc7ccccc7c6c45)cc3)c3ccc4cc5sc6ccccc6c5cc4c3)c2)cc1 ; c1ccc(-c2cccc(N(c3ccc(-c4cccc5c4ccc4ccc6ccccc6c45)cc3)c3cccc4cc5sc6ccccc6c5cc34)c2)cc1]

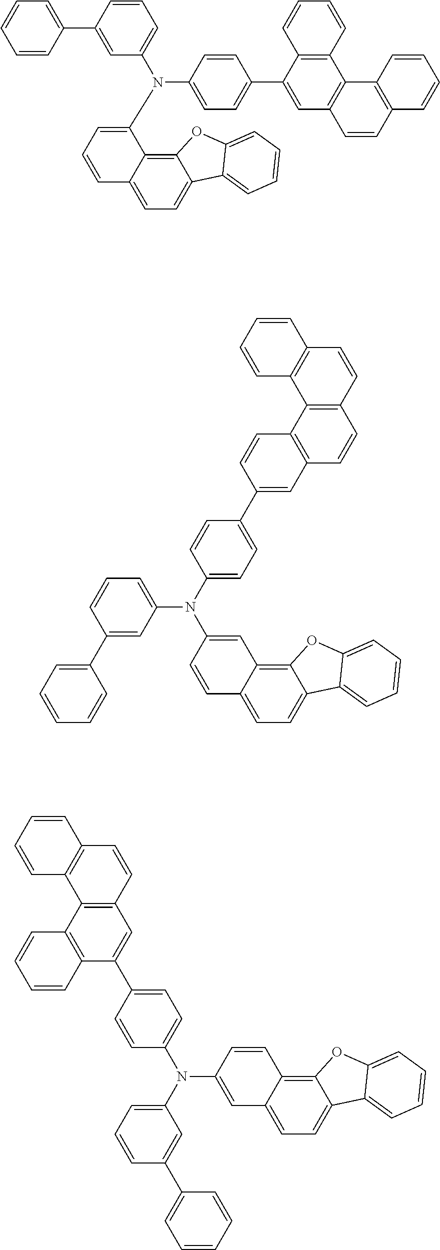 [SMILES: c1ccc(-c2cccc(N(c3ccc(-c4cc5ccc6ccccc6c5c5ccccc45)cc3)c3cccc4ccc5c6ccccc6oc5c34)c2)cc1 ; c1ccc(-c2cccc(N(c3ccc(-c4ccc5c(ccc6ccc7ccccc7c65)c4)cc3)c3ccc4ccc5c6ccccc6oc5c4c3)c2)cc1 ; c1ccc(-c2cccc(N(c3ccc(-c4cc5ccc6ccccc6c5c5ccccc45)cc3)c3ccc4c(ccc5c6ccccc6oc45)c3)c2)cc1]

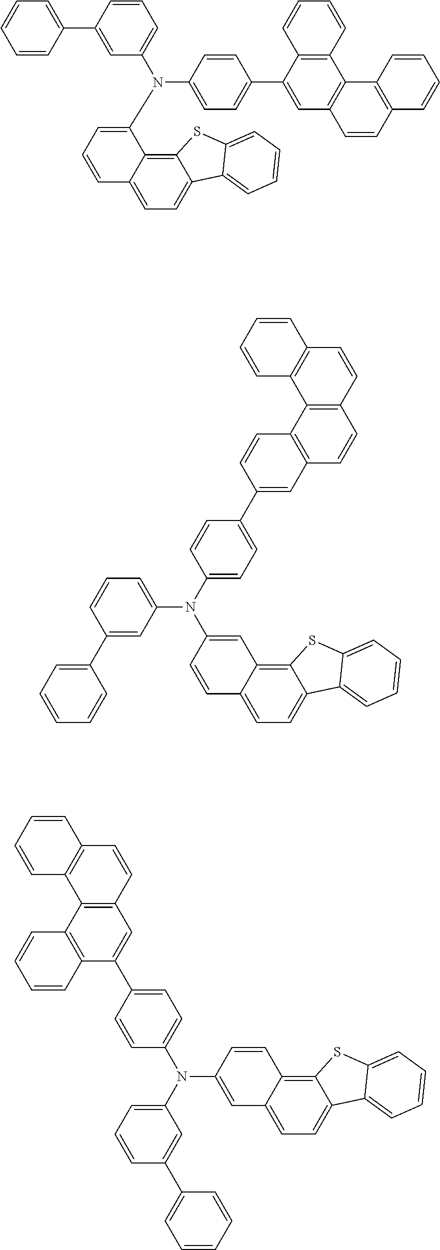 [SMILES: c1ccc(-c2cccc(N(c3ccc(-c4cc5ccc6ccccc6c5c5ccccc45)cc3)c3cccc4ccc5c6ccccc6sc5c34)c2)cc1 ; c1ccc(-c2cccc(N(c3ccc(-c4ccc5c(ccc6ccc7ccccc7c65)c4)cc3)c3ccc4ccc5c6ccccc6sc5c4c3)c2)cc1 ; c1ccc(-c2cccc(N(c3ccc(-c4cc5ccc6ccccc6c5c5ccccc45)cc3)c3ccc4c(ccc5c6ccccc6sc45)c3)c2)cc1]

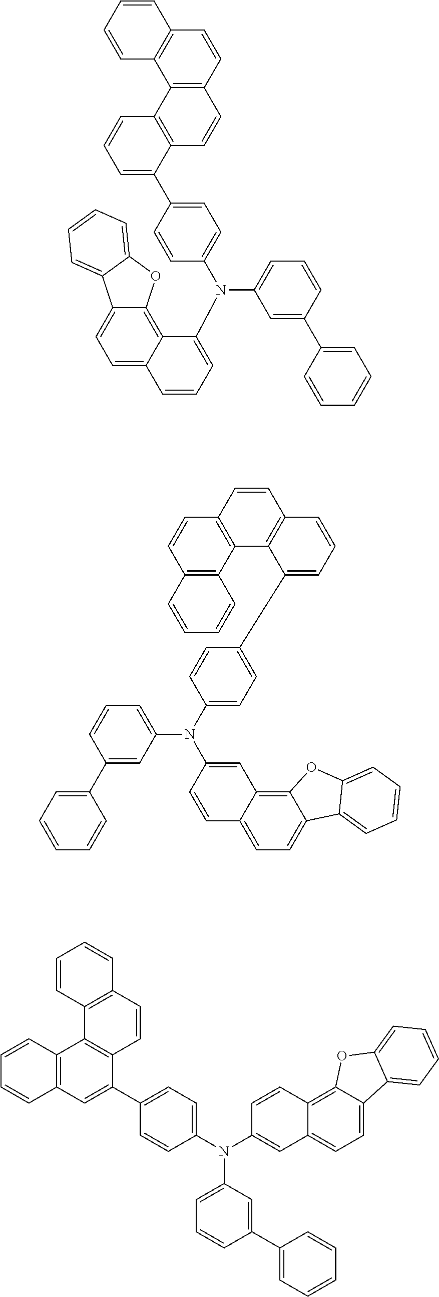 [SMILES: c1ccc(-c2cccc(N(c3ccc(-c4cccc5c4ccc4ccc6ccccc6c45)cc3)c3cccc4ccc5c6ccccc6oc5c34)c2)cc1 ; c1ccc(-c2cccc(N(c3ccc(-c4cccc5ccc6ccc7ccccc7c6c45)cc3)c3ccc4ccc5c6ccccc6oc5c4c3)c2)cc1 ; c1ccc(-c2cccc(N(c3ccc(-c4cc5ccccc5c5c4ccc4ccccc45)cc3)c3ccc4c(ccc5c6ccccc6oc45)c3)c2)cc1]

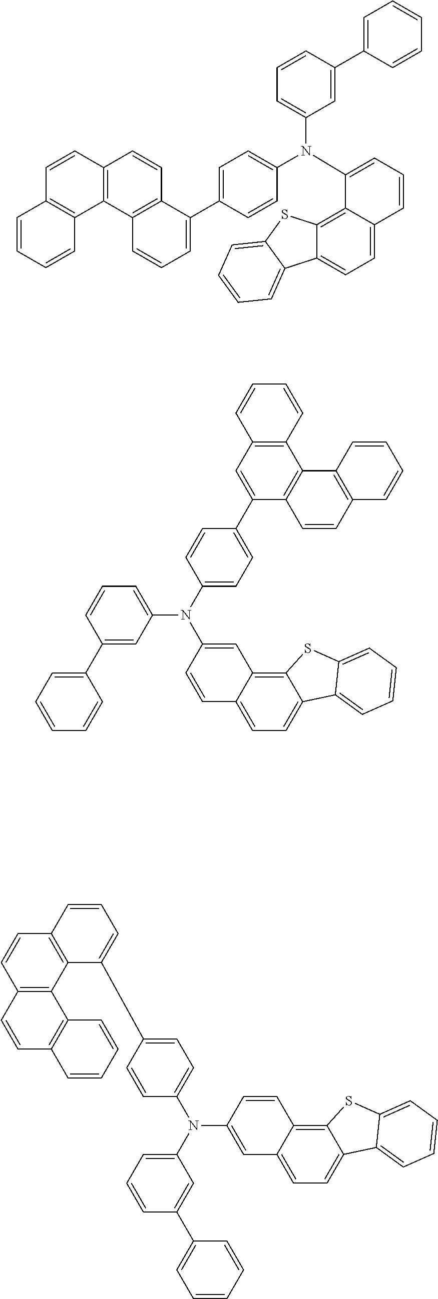 [SMILES: c1ccc(-c2cccc(N(c3ccc(-c4cccc5c4ccc4ccc6ccccc6c45)cc3)c3cccc4ccc5c6ccccc6sc5c34)c2)cc1 ; c1ccc(-c2cccc(N(c3ccc(-c4cc5ccccc5c5c4ccc4ccccc45)cc3)c3ccc4ccc5c6ccccc6sc5c4c3)c2)cc1 ; c1ccc(-c2cccc(N(c3ccc(-c4cccc5ccc6ccc7ccccc7c6c45)cc3)c3ccc4c(ccc5c6ccccc6sc45)c3)c2)cc1]

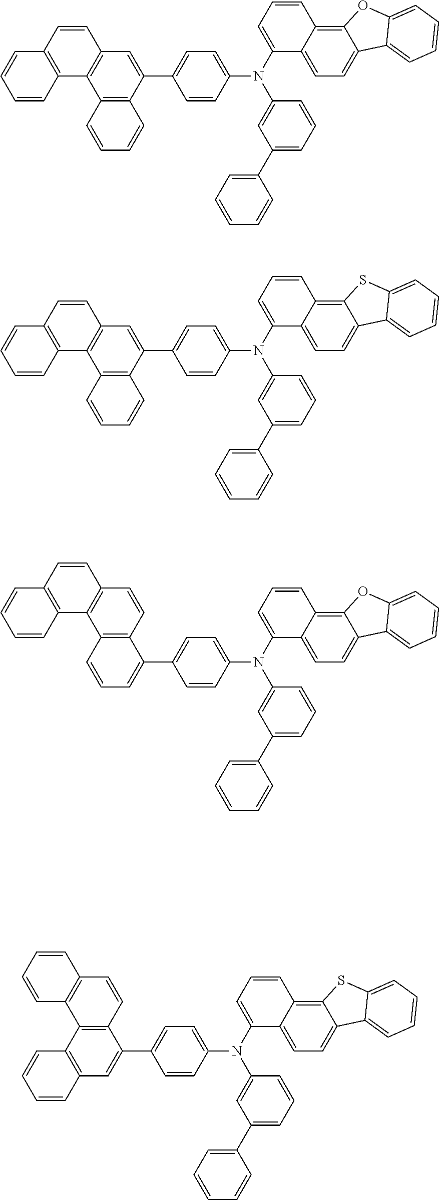 [SMILES: c1ccc(-c2cccc(N(c3ccc(-c4cc5ccc6ccccc6c5c5ccccc45)cc3)c3cccc4c3ccc3c5ccccc5oc43)c2)cc1 ; c1ccc(-c2cccc(N(c3ccc(-c4cc5ccc6ccccc6c5c5ccccc45)cc3)c3cccc4c3ccc3c5ccccc5sc43)c2)cc1 ; c1ccc(-c2cccc(N(c3ccc(-c4cccc5c4ccc4ccc6ccccc6c45)cc3)c3cccc4c3ccc3c5ccccc5oc43)c2)cc1 ; c1ccc(-c2cccc(N(c3ccc(-c4cc5ccccc5c5c4ccc4ccccc45)cc3)c3cccc4c3ccc3c5ccccc5sc43)c2)cc1]

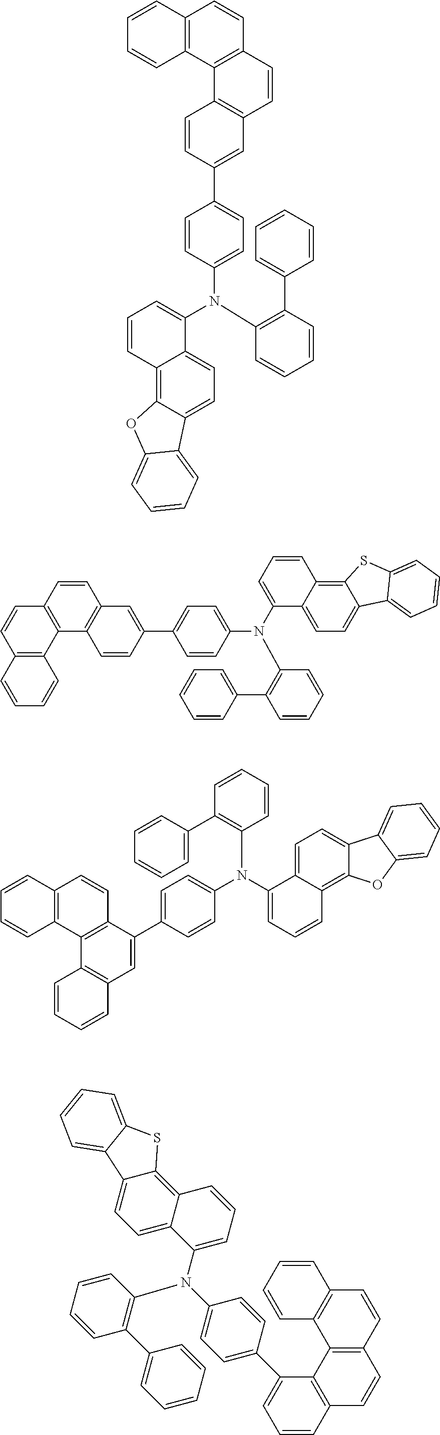 [SMILES: c1ccc(-c2ccccc2N(c2ccc(-c3ccc4c(ccc5ccc6ccccc6c54)c3)cc2)c2cccc3c2ccc2c4ccccc4oc32)cc1 ; c1ccc(-c2ccccc2N(c2ccc(-c3ccc4c(ccc5ccc6ccccc6c54)c3)cc2)c2cccc3c2ccc2c4ccccc4sc32)cc1 ; c1ccc(-c2ccccc2N(c2ccc(-c3cc4ccccc4c4c3ccc3ccccc34)cc2)c2cccc3c2ccc2c4ccccc4oc32)cc1 ; c1ccc(-c2ccccc2N(c2ccc(-c3cccc4ccc5ccc6ccccc6c5c34)cc2)c2cccc3c2ccc2c4ccccc4sc32)cc1]

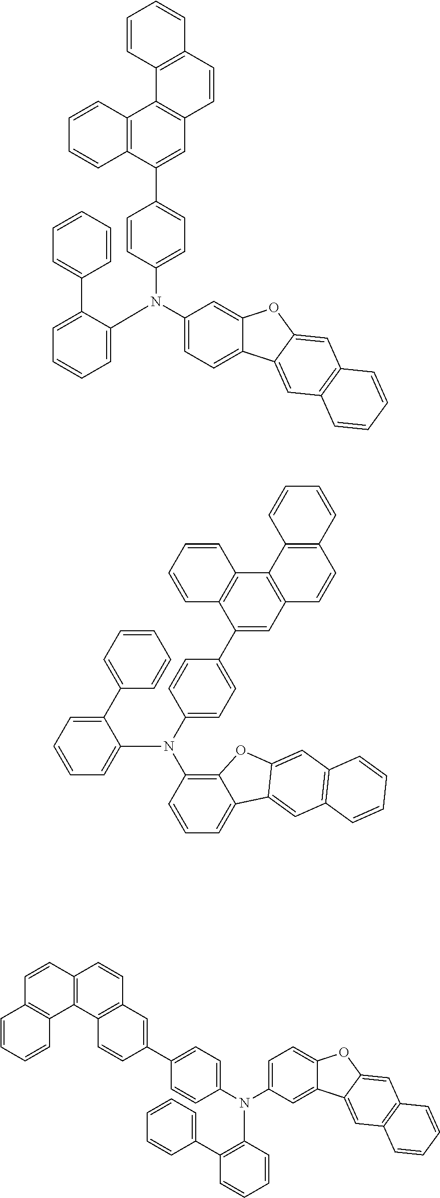 [SMILES: c1ccc(-c2ccccc2N(c2ccc(-c3cc4ccc5ccccc5c4c4ccccc34)cc2)c2ccc3c(c2)oc2cc4ccccc4cc23)cc1 ; c1ccc(-c2ccccc2N(c2ccc(-c3cc4ccc5ccccc5c4c4ccccc34)cc2)c2cccc3c2oc2cc4ccccc4cc23)cc1 ; c1ccc(-c2ccccc2N(c2ccc(-c3ccc4c(ccc5ccc6ccccc6c54)c3)cc2)c2ccc3oc4cc5ccccc5cc4c3c2)cc1]

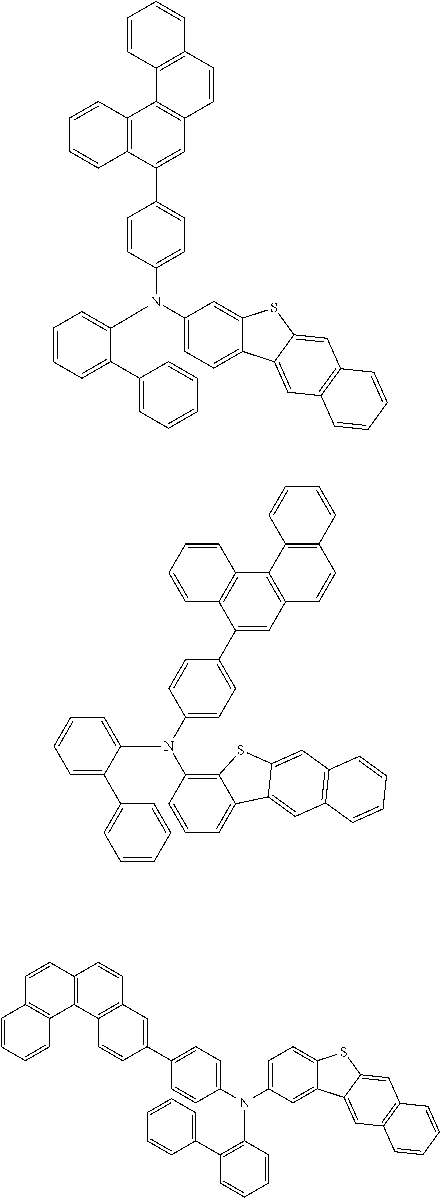 [SMILES: c1ccc(-c2ccccc2N(c2ccc(-c3cc4ccc5ccccc5c4c4ccccc34)cc2)c2ccc3c(c2)sc2cc4ccccc4cc23)cc1 ; c1ccc(-c2ccccc2N(c2ccc(-c3cc4ccc5ccccc5c4c4ccccc34)cc2)c2cccc3c2sc2cc4ccccc4cc23)cc1 ; c1ccc(-c2ccccc2N(c2ccc(-c3ccc4c(ccc5ccc6ccccc6c54)c3)cc2)c2ccc3sc4cc5ccccc5cc4c3c2)cc1]

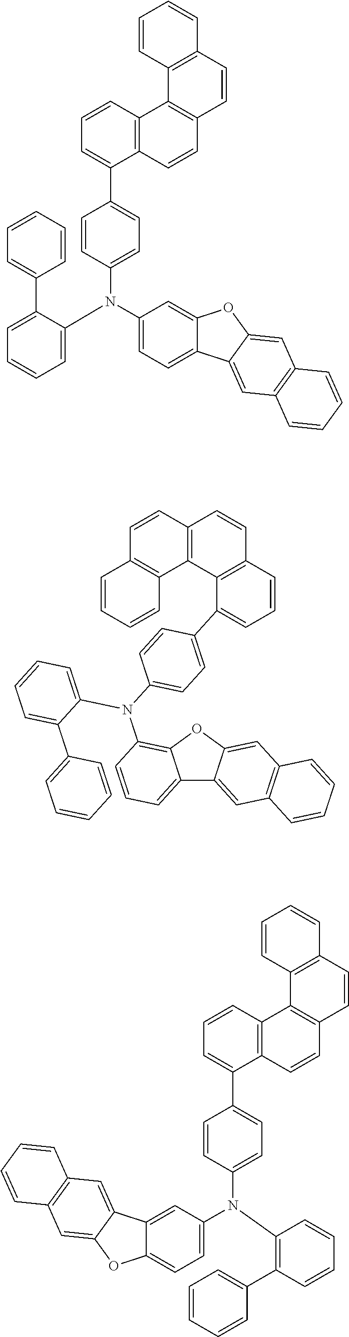 [SMILES: c1ccc(-c2ccccc2N(c2ccc(-c3cccc4c3ccc3ccc5ccccc5c34)cc2)c2ccc3c(c2)oc2cc4ccccc4cc23)cc1 ; c1ccc(-c2ccccc2N(c2ccc(-c3cccc4ccc5ccc6ccccc6c5c34)cc2)c2cccc3c2oc2cc4ccccc4cc23)cc1 ; c1ccc(-c2ccccc2N(c2ccc(-c3cccc4c3ccc3ccc5ccccc5c34)cc2)c2ccc3oc4cc5ccccc5cc4c3c2)cc1]

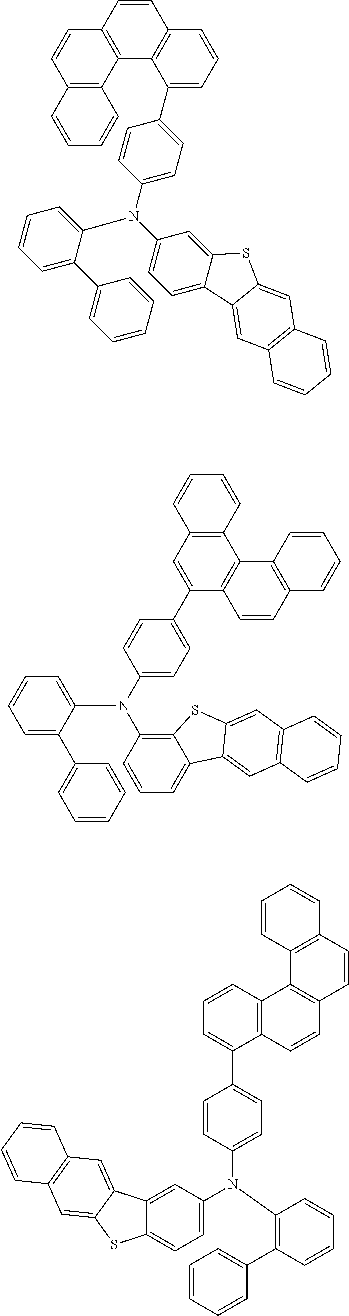 [SMILES: c1ccc(-c2ccccc2N(c2ccc(-c3cccc4ccc5ccc6ccccc6c5c34)cc2)c2ccc3c(c2)sc2cc4ccccc4cc23)cc1 ; c1ccc(-c2ccccc2N(c2ccc(-c3cc4ccccc4c4c3ccc3ccccc34)cc2)c2cccc3c2sc2cc4ccccc4cc23)cc1 ; c1ccc(-c2ccccc2N(c2ccc(-c3cccc4c3ccc3ccc5ccccc5c34)cc2)c2ccc3sc4cc5ccccc5cc4c3c2)cc1]

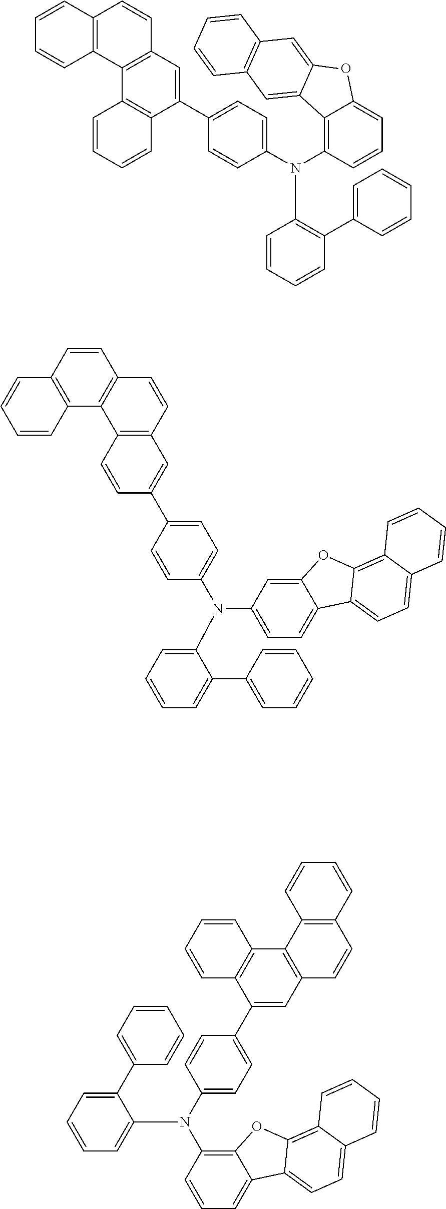 [SMILES: c1ccc(-c2ccccc2N(c2ccc(-c3cc4ccc5ccccc5c4c4ccccc34)cc2)c2cccc3oc4cc5ccccc5cc4c23)cc1 ; c1ccc(-c2ccccc2N(c2ccc(-c3ccc4c(ccc5ccc6ccccc6c54)c3)cc2)c2ccc3c(c2)oc2c4ccccc4ccc32)cc1 ; c1ccc(-c2ccccc2N(c2ccc(-c3cc4ccc5ccccc5c4c4ccccc34)cc2)c2cccc3c2oc2c4ccccc4ccc32)cc1]

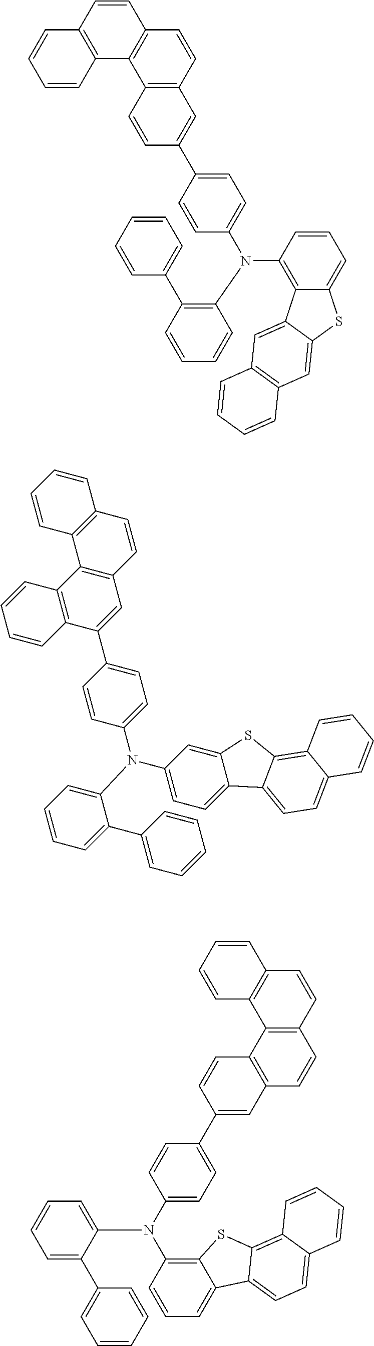 [SMILES: c1ccc(-c2ccccc2N(c2ccc(-c3ccc4c(ccc5ccc6ccccc6c54)c3)cc2)c2cccc3sc4cc5ccccc5cc4c23)cc1 ; c1ccc(-c2ccccc2N(c2ccc(-c3cc4ccc5ccccc5c4c4ccccc34)cc2)c2ccc3c(c2)sc2c4ccccc4ccc32)cc1 ; c1ccc(-c2ccccc2N(c2ccc(-c3ccc4c(ccc5ccc6ccccc6c54)c3)cc2)c2cccc3c2sc2c4ccccc4ccc32)cc1]

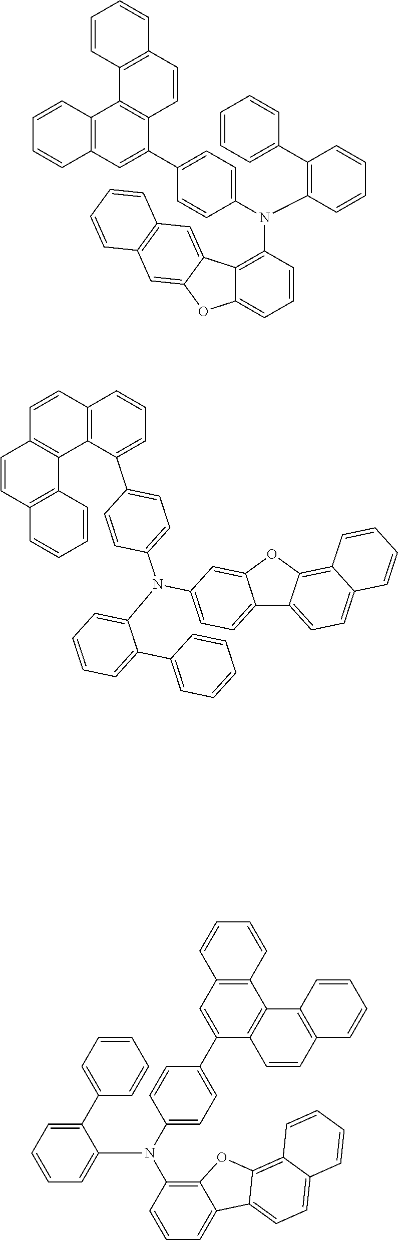 [SMILES: c1ccc(-c2ccccc2N(c2ccc(-c3cc4ccccc4c4c3ccc3ccccc34)cc2)c2cccc3oc4cc5ccccc5cc4c23)cc1 ; c1ccc(-c2ccccc2N(c2ccc(-c3cccc4ccc5ccc6ccccc6c5c34)cc2)c2ccc3c(c2)oc2c4ccccc4ccc32)cc1 ; c1ccc(-c2ccccc2N(c2ccc(-c3cc4ccccc4c4c3ccc3ccccc34)cc2)c2cccc3c2oc2c4ccccc4ccc32)cc1]

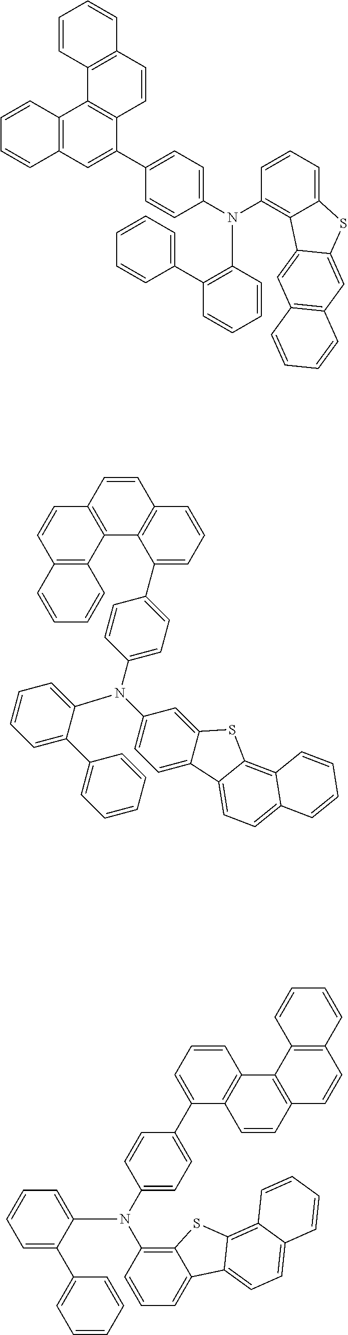 [SMILES: c1ccc(-c2ccccc2N(c2ccc(-c3cc4ccccc4c4c3ccc3ccccc34)cc2)c2cccc3sc4cc5ccccc5cc4c23)cc1 ; c1ccc(-c2ccccc2N(c2ccc(-c3cccc4ccc5ccc6ccccc6c5c34)cc2)c2ccc3c(c2)sc2c4ccccc4ccc32)cc1 ; c1ccc(-c2ccccc2N(c2ccc(-c3cccc4c3ccc3ccc5ccccc5c34)cc2)c2cccc3c2sc2c4ccccc4ccc32)cc1]

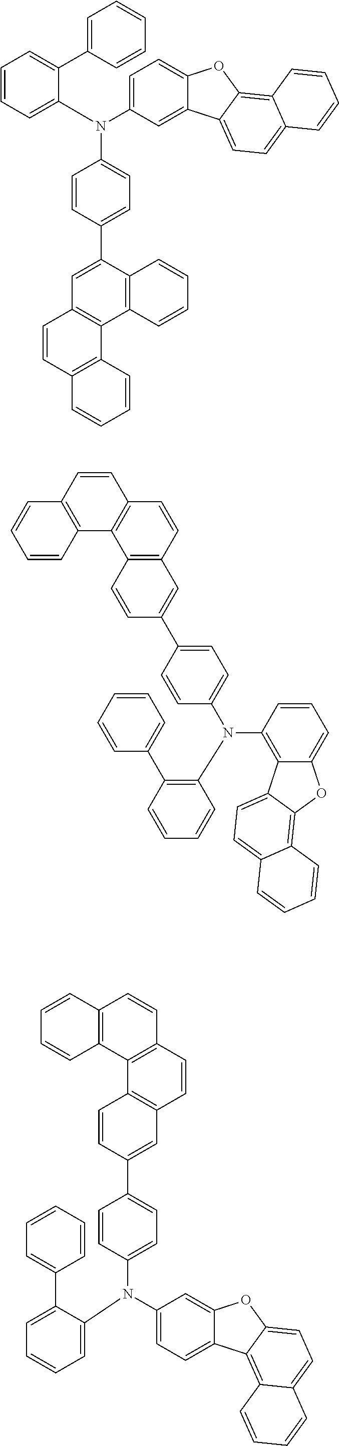 [SMILES: c1ccc(-c2ccccc2N(c2ccc(-c3cc4ccc5ccccc5c4c4ccccc34)cc2)c2ccc3oc4c5ccccc5ccc4c3c2)cc1 ; c1ccc(-c2ccccc2N(c2ccc(-c3ccc4c(ccc5ccc6ccccc6c54)c3)cc2)c2cccc3oc4c5ccccc5ccc4c23)cc1 ; c1ccc(-c2ccccc2N(c2ccc(-c3ccc4c(ccc5ccc6ccccc6c54)c3)cc2)c2ccc3c(c2)oc2ccc4ccccc4c23)cc1]

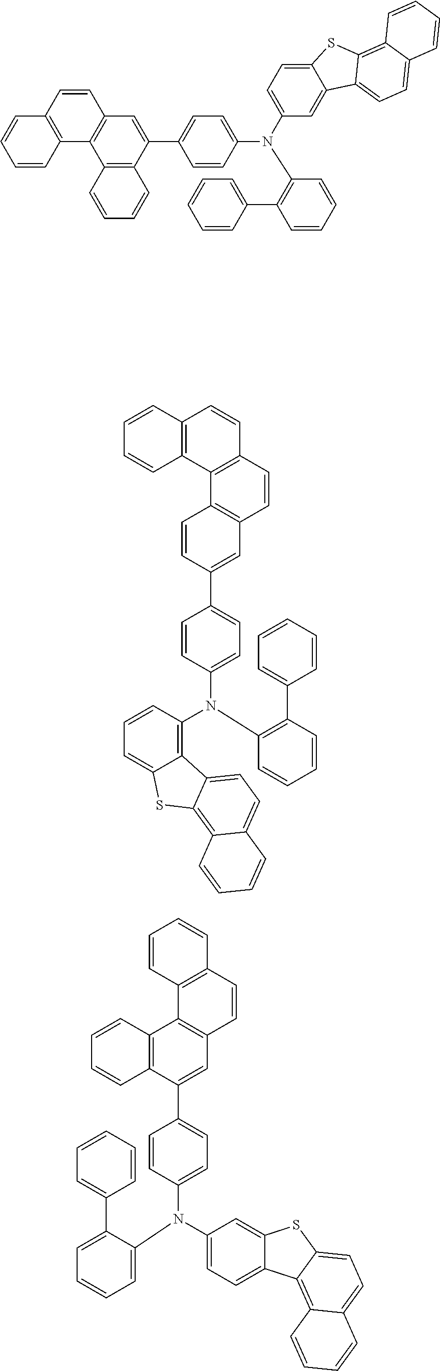

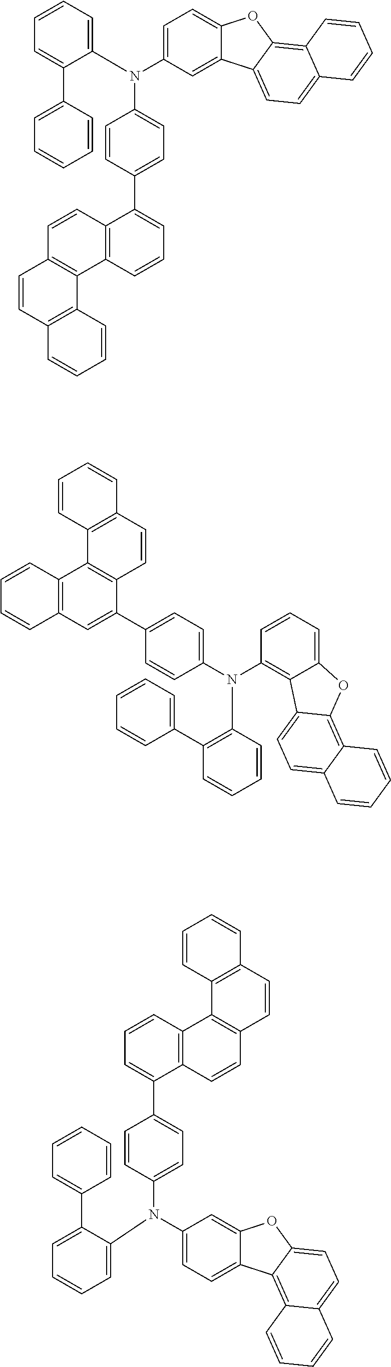 [SMILES: c1ccc(-c2ccccc2N(c2ccc(-c3cccc4c3ccc3ccc5ccccc5c34)cc2)c2ccc3oc4c5ccccc5ccc4c3c2)cc1 ; c1ccc(-c2ccccc2N(c2ccc(-c3cc4ccccc4c4c3ccc3ccccc34)cc2)c2cccc3oc4c5ccccc5ccc4c23)cc1 ; c1ccc(-c2ccccc2N(c2ccc(-c3cccc4c3ccc3ccc5ccccc5c34)cc2)c2ccc3c(c2)oc2ccc4ccccc4c23)cc1]

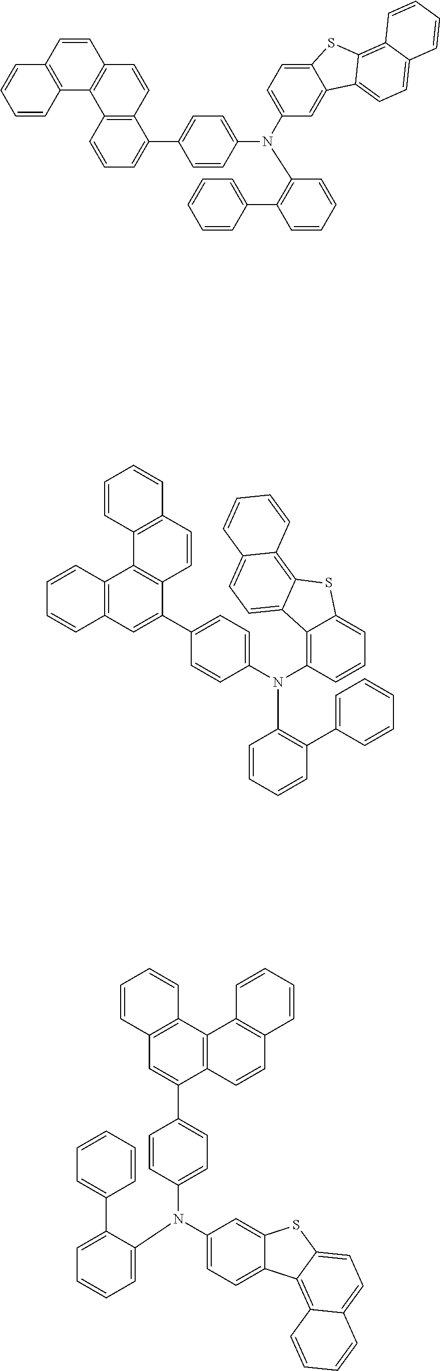 [SMILES: c1ccc(-c2ccccc2N(c2ccc(-c3cccc4c3ccc3ccc5ccccc5c34)cc2)c2ccc3sc4c5ccccc5ccc4c3c2)cc1 ; c1ccc(-c2ccccc2N(c2ccc(-c3cc4ccccc4c4c3ccc3ccccc34)cc2)c2cccc3sc4c5ccccc5ccc4c23)cc1 ; c1ccc(-c2ccccc2N(c2ccc(-c3cc4ccccc4c4c3ccc3ccccc34)cc2)c2ccc3c(c2)sc2ccc4ccccc4c23)cc1]

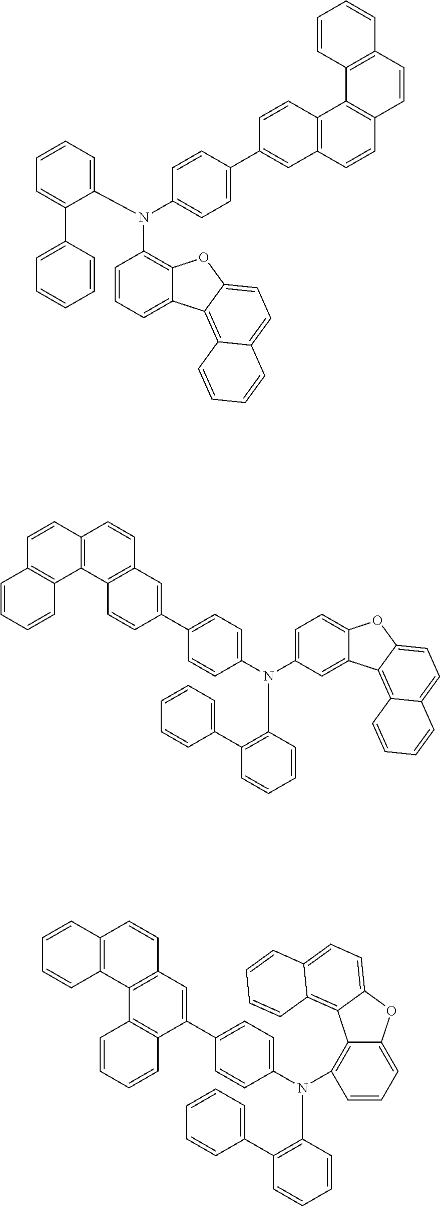 [SMILES: c1ccc(-c2ccccc2N(c2ccc(-c3ccc4c(ccc5ccc6ccccc6c54)c3)cc2)c2cccc3c2oc2ccc4ccccc4c23)cc1 ; c1ccc(-c2ccccc2N(c2ccc(-c3ccc4c(ccc5ccc6ccccc6c54)c3)cc2)c2ccc3oc4ccc5ccccc5c4c3c2)cc1 ; c1ccc(-c2ccccc2N(c2ccc(-c3cc4ccc5ccccc5c4c4ccccc34)cc2)c2cccc3oc4ccc5ccccc5c4c23)cc1]

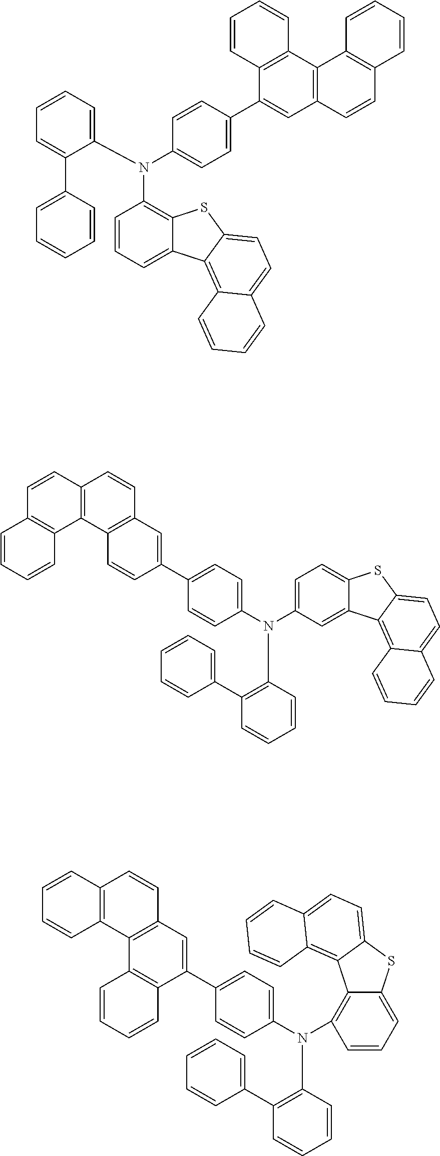 [SMILES: c1ccc(-c2ccccc2N(c2ccc(-c3cc4ccc5ccccc5c4c4ccccc34)cc2)c2cccc3c2sc2ccc4ccccc4c23)cc1 ; c1ccc(-c2ccccc2N(c2ccc(-c3ccc4c(ccc5ccc6ccccc6c54)c3)cc2)c2ccc3sc4ccc5ccccc5c4c3c2)cc1 ; c1ccc(-c2ccccc2N(c2ccc(-c3cc4ccc5ccccc5c4c4ccccc34)cc2)c2cccc3sc4ccc5ccccc5c4c23)cc1]

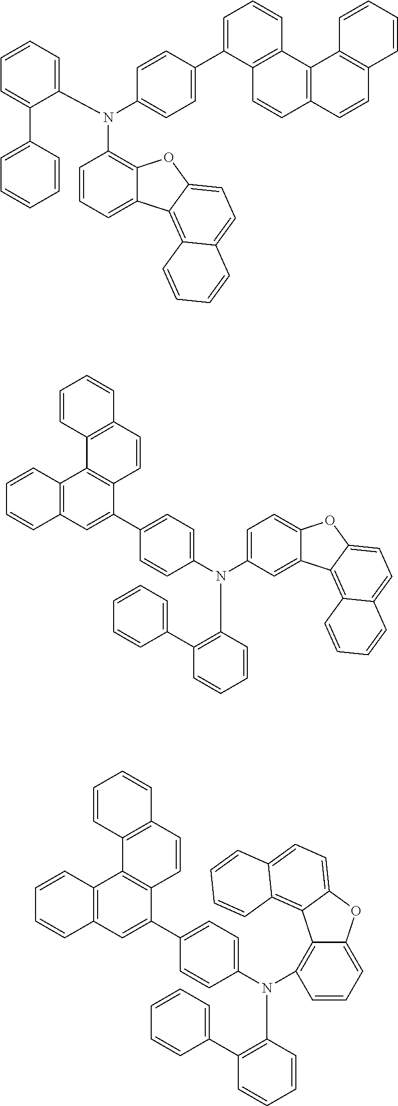 [SMILES: c1ccc(-c2ccccc2N(c2ccc(-c3cccc4c3ccc3ccc5ccccc5c34)cc2)c2cccc3c2oc2ccc4ccccc4c23)cc1 ; c1ccc(-c2ccccc2N(c2ccc(-c3cc4ccccc4c4c3ccc3ccccc34)cc2)c2ccc3oc4ccc5ccccc5c4c3c2)cc1 ; c1ccc(-c2ccccc2N(c2ccc(-c3cc4ccccc4c4c3ccc3ccccc34)cc2)c2cccc3oc4ccc5ccccc5c4c23)cc1]

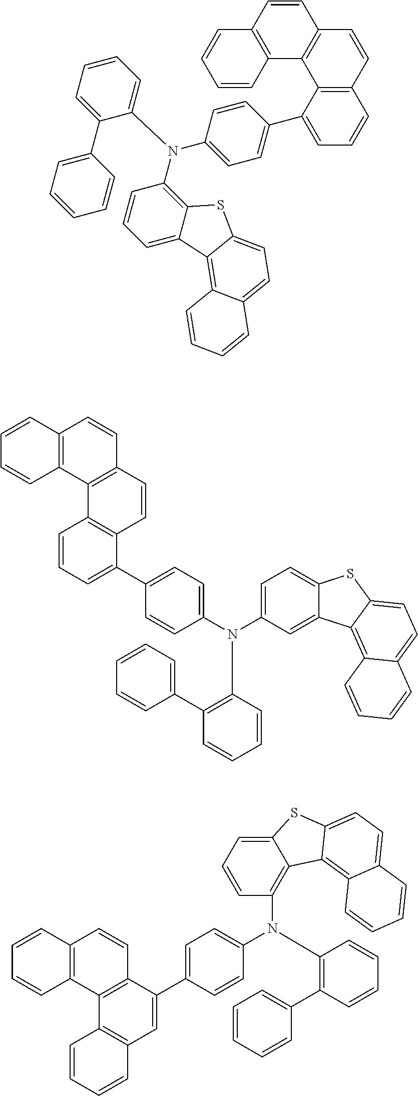 [SMILES: c1ccc(-c2ccccc2N(c2ccc(-c3cccc4ccc5ccc6ccccc6c5c34)cc2)c2cccc3c2sc2ccc4ccccc4c23)cc1 ; c1ccc(-c2ccccc2N(c2ccc(-c3cccc4c3ccc3ccc5ccccc5c34)cc2)c2ccc3sc4ccc5ccccc5c4c3c2)cc1 ; c1ccc(-c2ccccc2N(c2ccc(-c3cc4ccccc4c4c3ccc3ccccc34)cc2)c2cccc3sc4ccc5ccccc5c4c23)cc1]

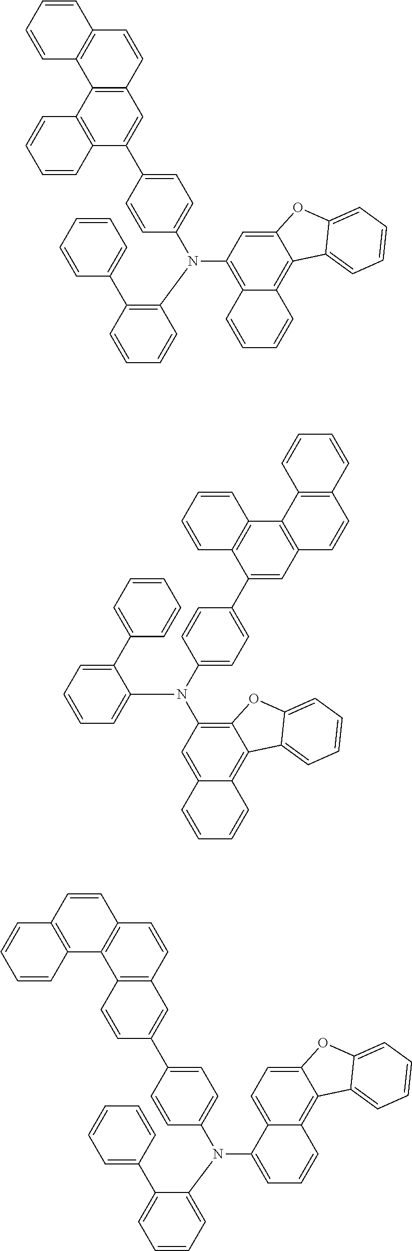 [SMILES: c1ccc(-c2ccccc2N(c2ccc(-c3cc4ccc5ccccc5c4c4ccccc34)cc2)c2cc3oc4ccccc4c3c3ccccc23)cc1 ; c1ccc(-c2ccccc2N(c2ccc(-c3cc4ccc5ccccc5c4c4ccccc34)cc2)c2cc3ccccc3c3c2oc2ccccc23)cc1 ; c1ccc(-c2ccccc2N(c2ccc(-c3ccc4c(ccc5ccc6ccccc6c54)c3)cc2)c2cccc3c2ccc2oc4ccccc4c23)cc1]

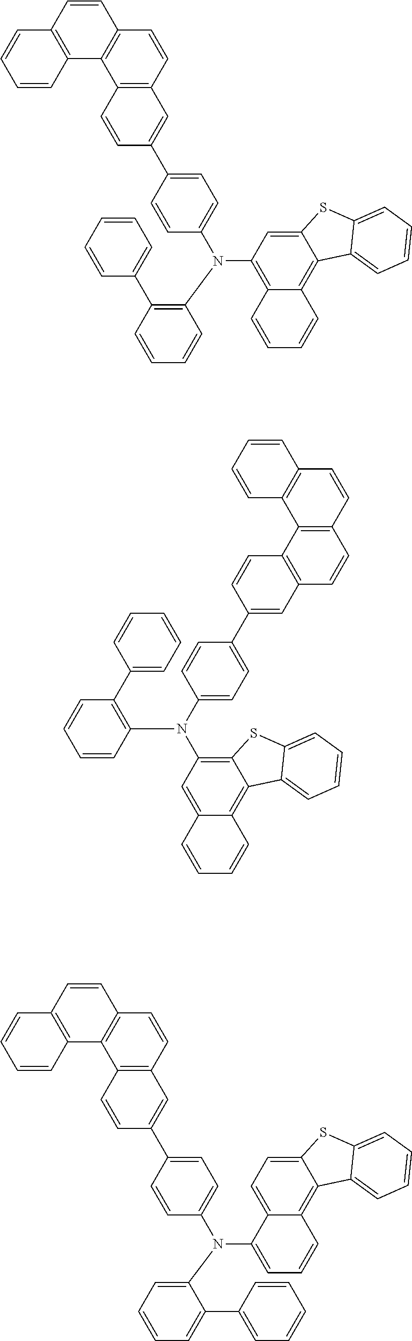 [SMILES: c1ccc(-c2ccccc2N(c2ccc(-c3ccc4c(ccc5ccc6ccccc6c54)c3)cc2)c2cc3sc4ccccc4c3c3ccccc23)cc1 ; c1ccc(-c2ccccc2N(c2ccc(-c3ccc4c(ccc5ccc6ccccc6c54)c3)cc2)c2cc3ccccc3c3c2sc2ccccc23)cc1 ; c1ccc(-c2ccccc2N(c2ccc(-c3ccc4c(ccc5ccc6ccccc6c54)c3)cc2)c2cccc3c2ccc2sc4ccccc4c23)cc1]

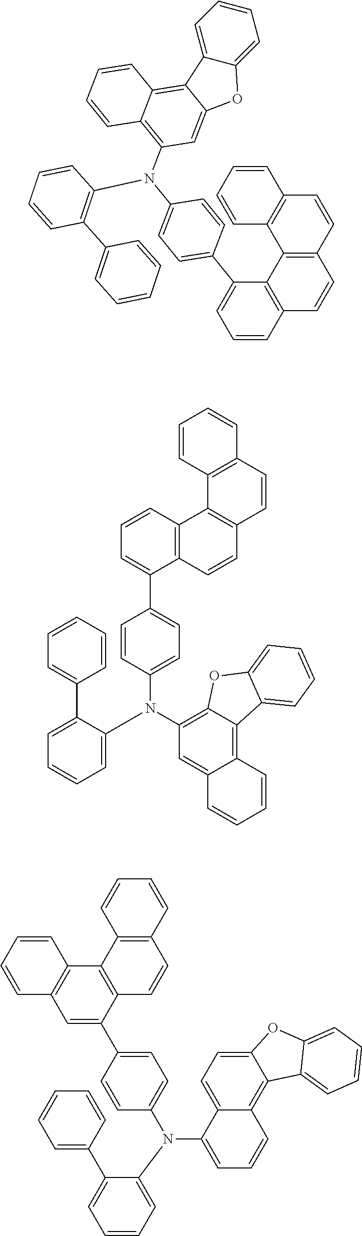 [SMILES: c1ccc(-c2ccccc2N(c2ccc(-c3cccc4ccc5ccc6ccccc6c5c34)cc2)c2cc3oc4ccccc4c3c3ccccc23)cc1 ; c1ccc(-c2ccccc2N(c2ccc(-c3cccc4c3ccc3ccc5ccccc5c34)cc2)c2cc3ccccc3c3c2oc2ccccc23)cc1 ; c1ccc(-c2ccccc2N(c2ccc(-c3cc4ccccc4c4c3ccc3ccccc34)cc2)c2cccc3c2ccc2oc4ccccc4c23)cc1]

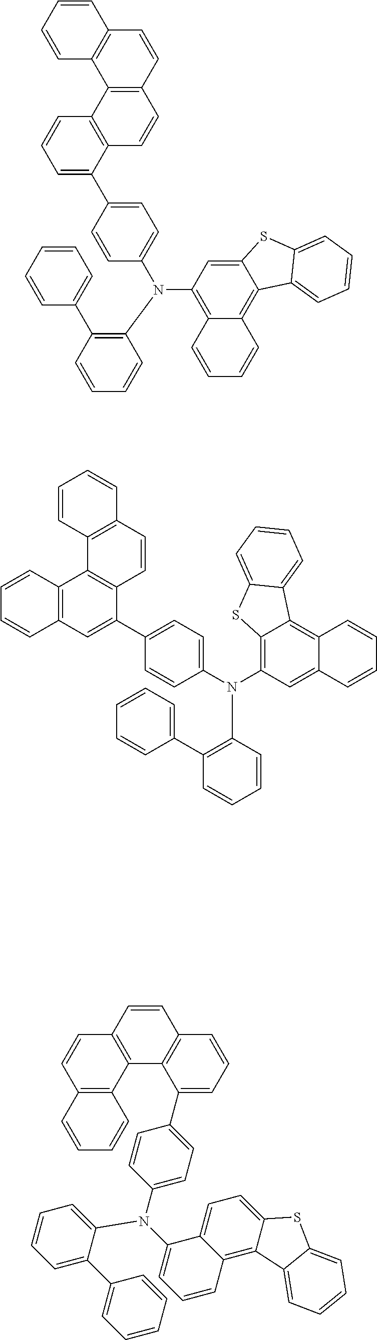 [SMILES: c1ccc(-c2ccccc2N(c2ccc(-c3cccc4c3ccc3ccc5ccccc5c34)cc2)c2cc3sc4ccccc4c3c3ccccc23)cc1 ; c1ccc(-c2ccccc2N(c2ccc(-c3cc4ccccc4c4c3ccc3ccccc34)cc2)c2cc3ccccc3c3c2sc2ccccc23)cc1 ; c1ccc(-c2ccccc2N(c2ccc(-c3cccc4ccc5ccc6ccccc6c5c34)cc2)c2cccc3c2ccc2sc4ccccc4c23)cc1]

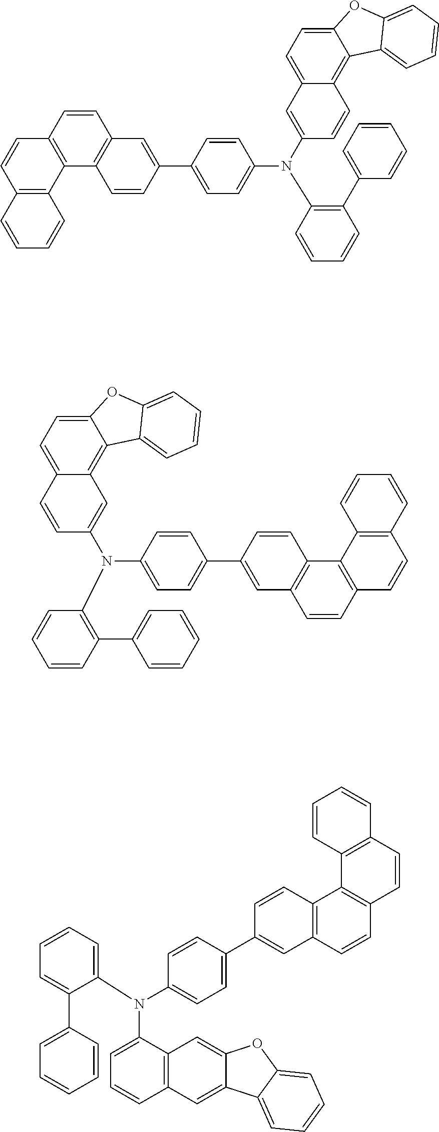 [SMILES: c1ccc(-c2ccccc2N(c2ccc(-c3ccc4c(ccc5ccc6ccccc6c54)c3)cc2)c2ccc3c(ccc4oc5ccccc5c43)c2)cc1 ; c1ccc(-c2ccccc2N(c2ccc(-c3ccc4c(ccc5ccc6ccccc6c54)c3)cc2)c2ccc3ccc4oc5ccccc5c4c3c2)cc1 ; c1ccc(-c2ccccc2N(c2ccc(-c3ccc4c(ccc5ccc6ccccc6c54)c3)cc2)c2cccc3cc4c(cc23)oc2ccccc24)cc1]

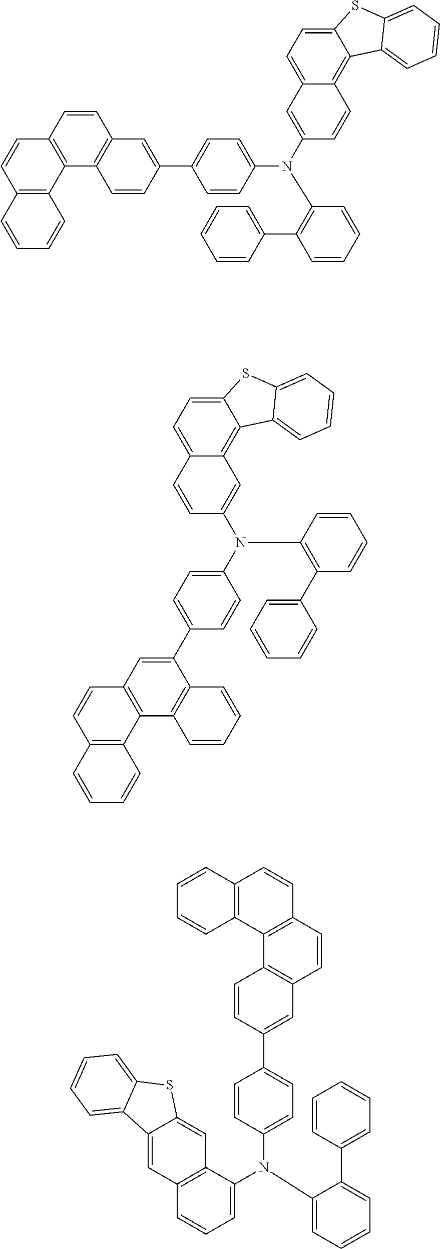 [SMILES: c1ccc(-c2ccccc2N(c2ccc(-c3ccc4c(ccc5ccc6ccccc6c54)c3)cc2)c2ccc3c(ccc4sc5ccccc5c43)c2)cc1 ; c1ccc(-c2ccccc2N(c2ccc(-c3cc4ccc5ccccc5c4c4ccccc34)cc2)c2ccc3ccc4sc5ccccc5c4c3c2)cc1 ; c1ccc(-c2ccccc2N(c2ccc(-c3ccc4c(ccc5ccc6ccccc6c54)c3)cc2)c2cccc3cc4c(cc23)sc2ccccc24)cc1]

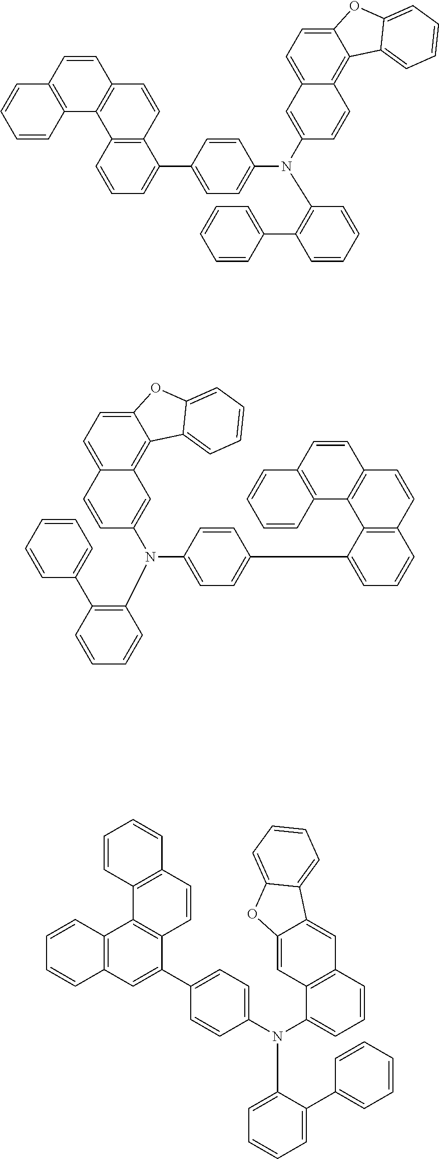 [SMILES: c1ccc(-c2ccccc2N(c2ccc(-c3cccc4c3ccc3ccc5ccccc5c34)cc2)c2ccc3c(ccc4oc5ccccc5c43)c2)cc1 ; c1ccc(-c2ccccc2N(c2ccc(-c3cccc4ccc5ccc6ccccc6c5c34)cc2)c2ccc3ccc4oc5ccccc5c4c3c2)cc1 ; c1ccc(-c2ccccc2N(c2ccc(-c3cc4ccccc4c4c3ccc3ccccc34)cc2)c2cccc3cc4c(cc23)oc2ccccc24)cc1]

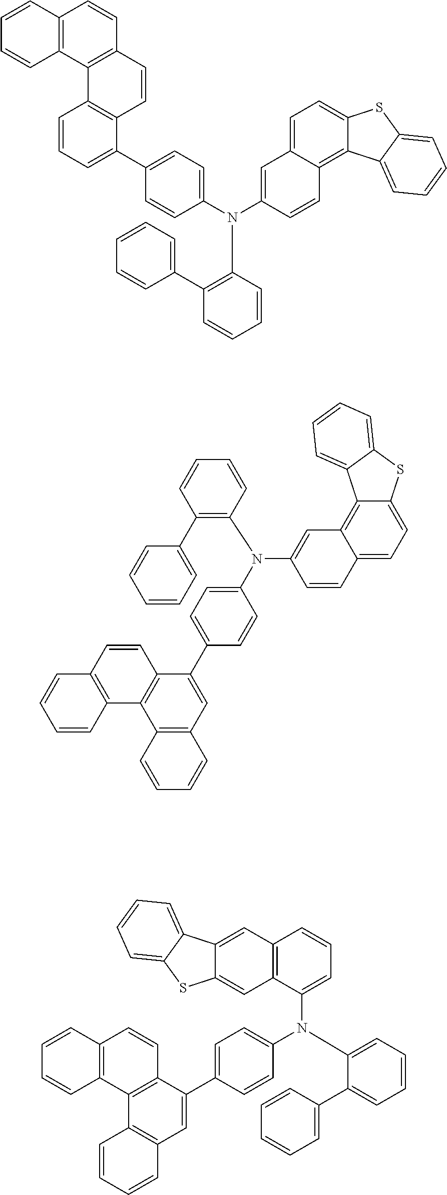 [SMILES: c1ccc(-c2ccccc2N(c2ccc(-c3cccc4c3ccc3ccc5ccccc5c34)cc2)c2ccc3c(ccc4sc5ccccc5c43)c2)cc1 ; c1ccc(-c2ccccc2N(c2ccc(-c3cc4ccccc4c4c3ccc3ccccc34)cc2)c2ccc3ccc4sc5ccccc5c4c3c2)cc1 ; c1ccc(-c2ccccc2N(c2ccc(-c3cc4ccccc4c4c3ccc3ccccc34)cc2)c2cccc3cc4c(cc23)sc2ccccc24)cc1]

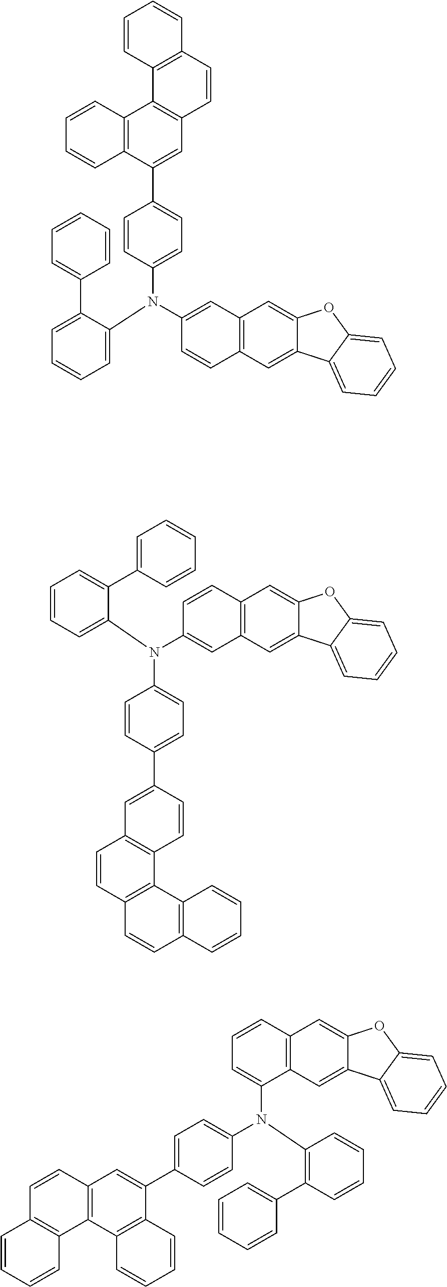 [SMILES: c1ccc(-c2ccccc2N(c2ccc(-c3cc4ccc5ccccc5c4c4ccccc34)cc2)c2ccc3cc4c(cc3c2)oc2ccccc24)cc1 ; c1ccc(-c2ccccc2N(c2ccc(-c3ccc4c(ccc5ccc6ccccc6c54)c3)cc2)c2ccc3cc4oc5ccccc5c4cc3c2)cc1 ; c1ccc(-c2ccccc2N(c2ccc(-c3cc4ccc5ccccc5c4c4ccccc34)cc2)c2cccc3cc4oc5ccccc5c4cc23)cc1]

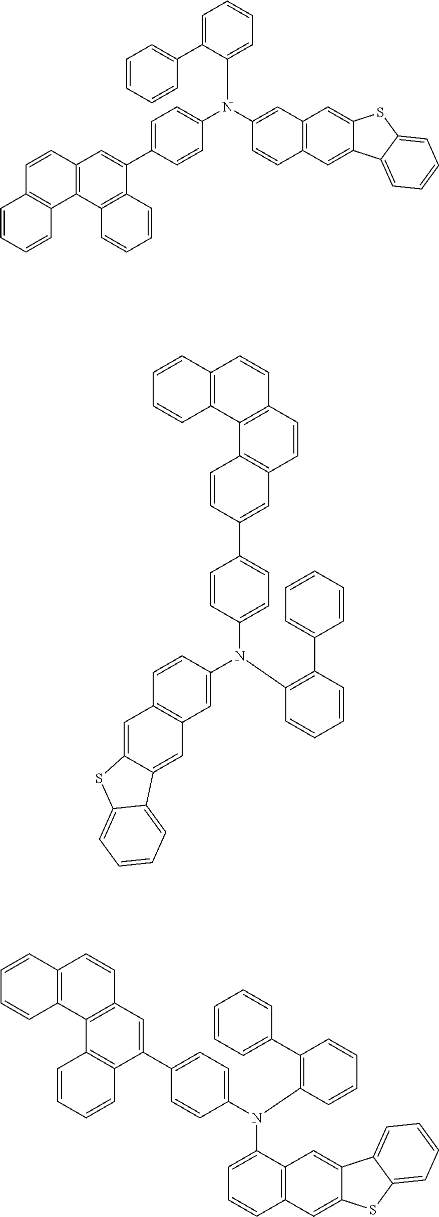 [SMILES: c1ccc(-c2ccccc2N(c2ccc(-c3cc4ccc5ccccc5c4c4ccccc34)cc2)c2ccc3cc4c(cc3c2)sc2ccccc24)cc1 ; c1ccc(-c2ccccc2N(c2ccc(-c3ccc4c(ccc5ccc6ccccc6c54)c3)cc2)c2ccc3cc4sc5ccccc5c4cc3c2)cc1 ; c1ccc(-c2ccccc2N(c2ccc(-c3cc4ccc5ccccc5c4c4ccccc34)cc2)c2cccc3cc4sc5ccccc5c4cc23)cc1]

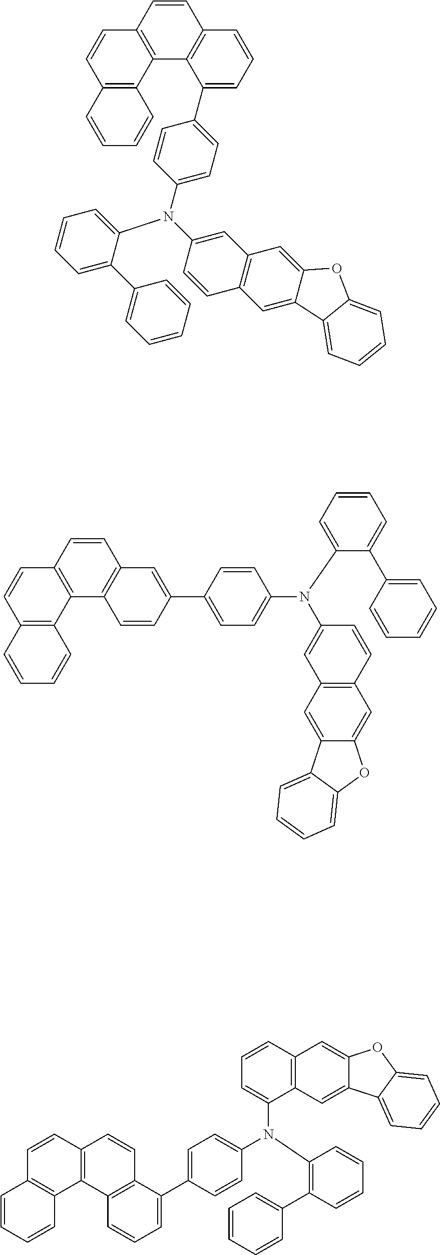 [SMILES: c1ccc(-c2ccccc2N(c2ccc(-c3cccc4ccc5ccc6ccccc6c5c34)cc2)c2ccc3cc4c(cc3c2)oc2ccccc24)cc1 ; c1ccc(-c2ccccc2N(c2ccc(-c3ccc4c(ccc5ccc6ccccc6c54)c3)cc2)c2ccc3cc4oc5ccccc5c4cc3c2)cc1 ; c1ccc(-c2ccccc2N(c2ccc(-c3cccc4c3ccc3ccc5ccccc5c34)cc2)c2cccc3cc4oc5ccccc5c4cc23)cc1]

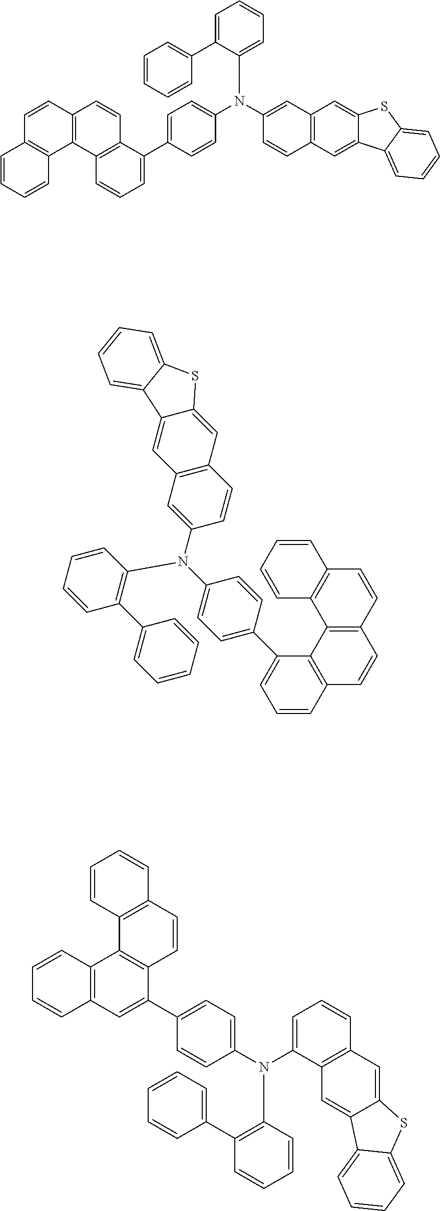 [SMILES: c1ccc(-c2ccccc2N(c2ccc(-c3cccc4c3ccc3ccc5ccccc5c34)cc2)c2ccc3cc4c(cc3c2)sc2ccccc24)cc1 ; c1ccc(-c2ccccc2N(c2ccc(-c3cccc4ccc5ccc6ccccc6c5c34)cc2)c2ccc3cc4sc5ccccc5c4cc3c2)cc1 ; c1ccc(-c2ccccc2N(c2ccc(-c3cc4ccccc4c4c3ccc3ccccc34)cc2)c2cccc3cc4sc5ccccc5c4cc23)cc1]

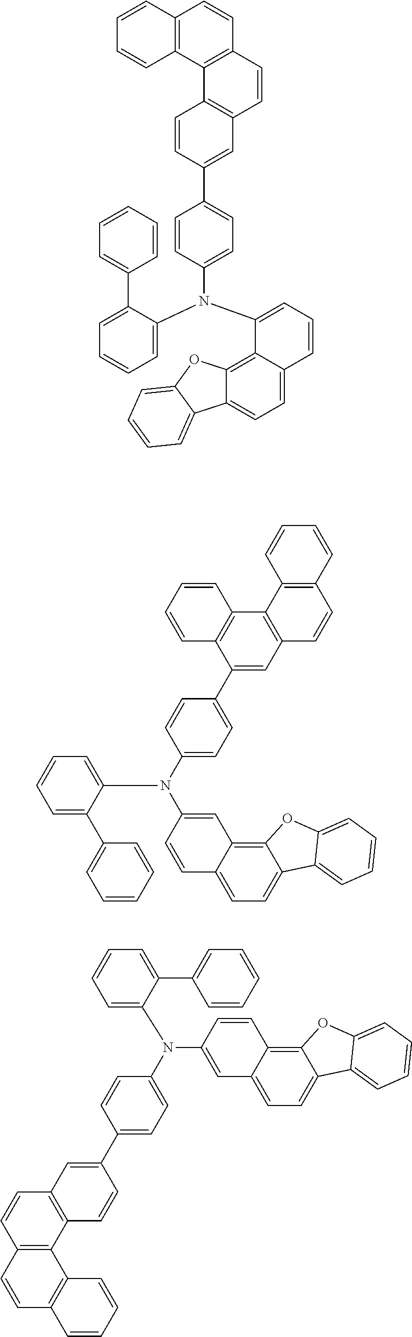 [SMILES: c1ccc(-c2ccccc2N(c2ccc(-c3ccc4c(ccc5ccc6ccccc6c54)c3)cc2)c2cccc3ccc4c5ccccc5oc4c23)cc1 ; c1ccc(-c2ccccc2N(c2ccc(-c3cc4ccc5ccccc5c4c4ccccc34)cc2)c2ccc3ccc4c5ccccc5oc4c3c2)cc1 ; c1ccc(-c2ccccc2N(c2ccc(-c3ccc4c(ccc5ccc6ccccc6c54)c3)cc2)c2ccc3c(ccc4c5ccccc5oc34)c2)cc1]

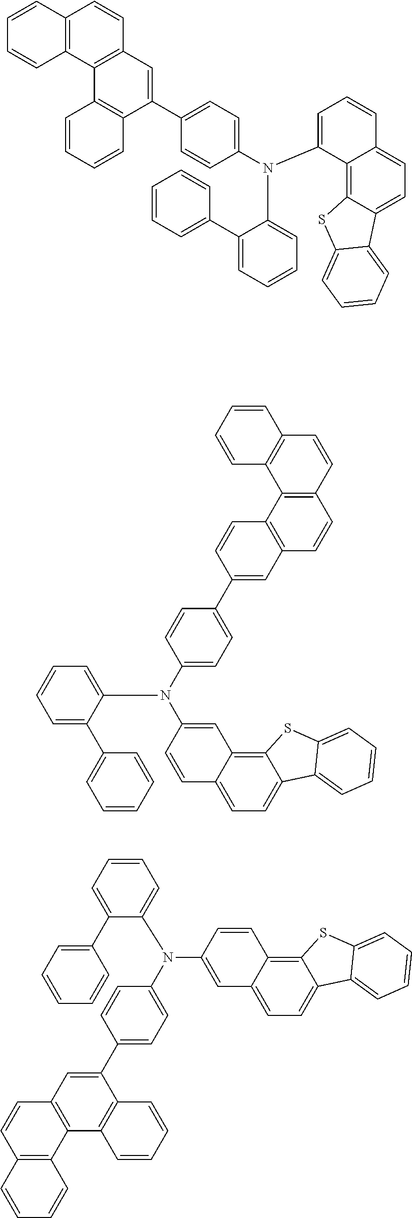 [SMILES: c1ccc(-c2ccccc2N(c2ccc(-c3cc4ccc5ccccc5c4c4ccccc34)cc2)c2cccc3ccc4c5ccccc5sc4c23)cc1 ; c1ccc(-c2ccccc2N(c2ccc(-c3ccc4c(ccc5ccc6ccccc6c54)c3)cc2)c2ccc3ccc4c5ccccc5sc4c3c2)cc1 ; c1ccc(-c2ccccc2N(c2ccc(-c3cc4ccc5ccccc5c4c4ccccc34)cc2)c2ccc3c(ccc4c5ccccc5sc34)c2)cc1]

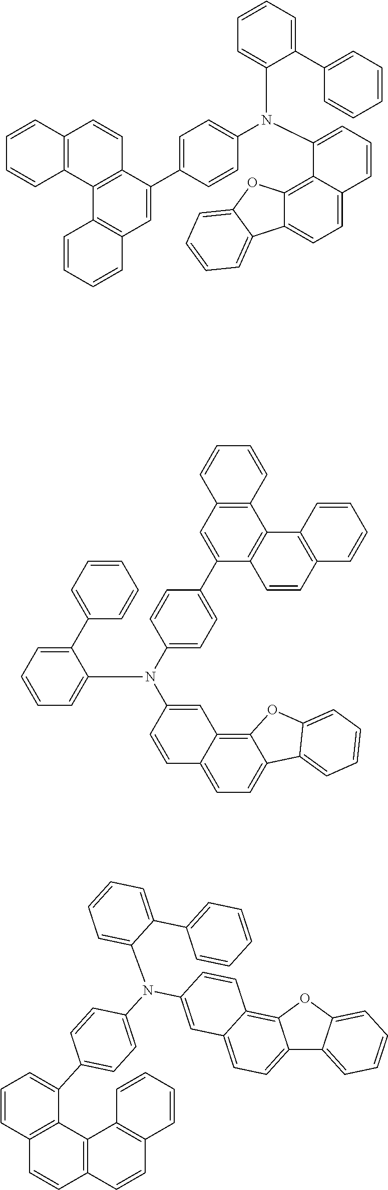 [SMILES: c1ccc(-c2ccccc2N(c2ccc(-c3cc4ccccc4c4c3ccc3ccccc34)cc2)c2cccc3ccc4c5ccccc5oc4c23)cc1 ; c1ccc(-c2ccccc2N(c2ccc(-c3cc4ccccc4c4c3ccc3ccccc34)cc2)c2ccc3ccc4c5ccccc5oc4c3c2)cc1 ; c1ccc(-c2ccccc2N(c2ccc(-c3cccc4ccc5ccc6ccccc6c5c34)cc2)c2ccc3c(ccc4c5ccccc5oc34)c2)cc1]

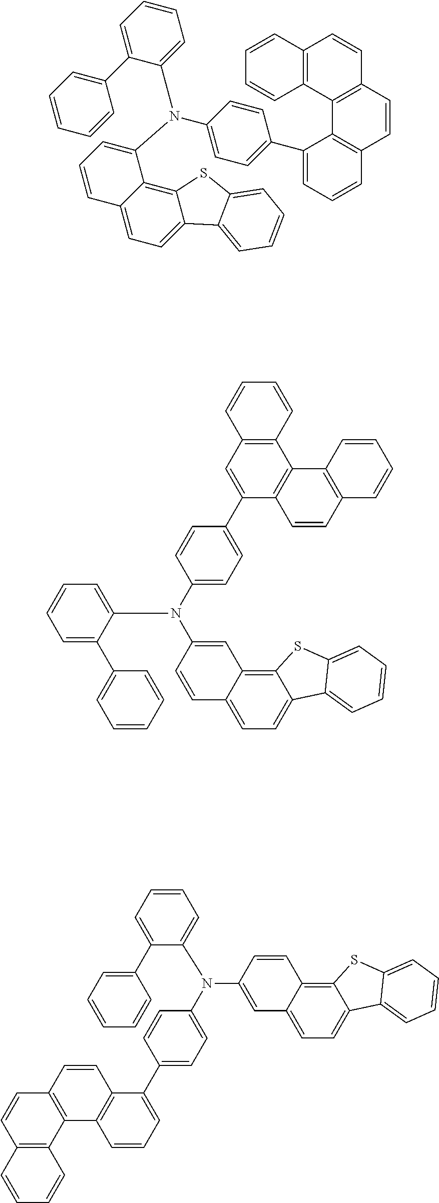 [SMILES: c1ccc(-c2ccccc2N(c2ccc(-c3cccc4ccc5ccc6ccccc6c5c34)cc2)c2cccc3ccc4c5ccccc5sc4c23)cc1 ; c1ccc(-c2ccccc2N(c2ccc(-c3cc4ccccc4c4c3ccc3ccccc34)cc2)c2ccc3ccc4c5ccccc5sc4c3c2)cc1 ; c1ccc(-c2ccccc2N(c2ccc(-c3cccc4c3ccc3ccc5ccccc5c34)cc2)c2ccc3c(ccc4c5ccccc5sc34)c2)cc1]

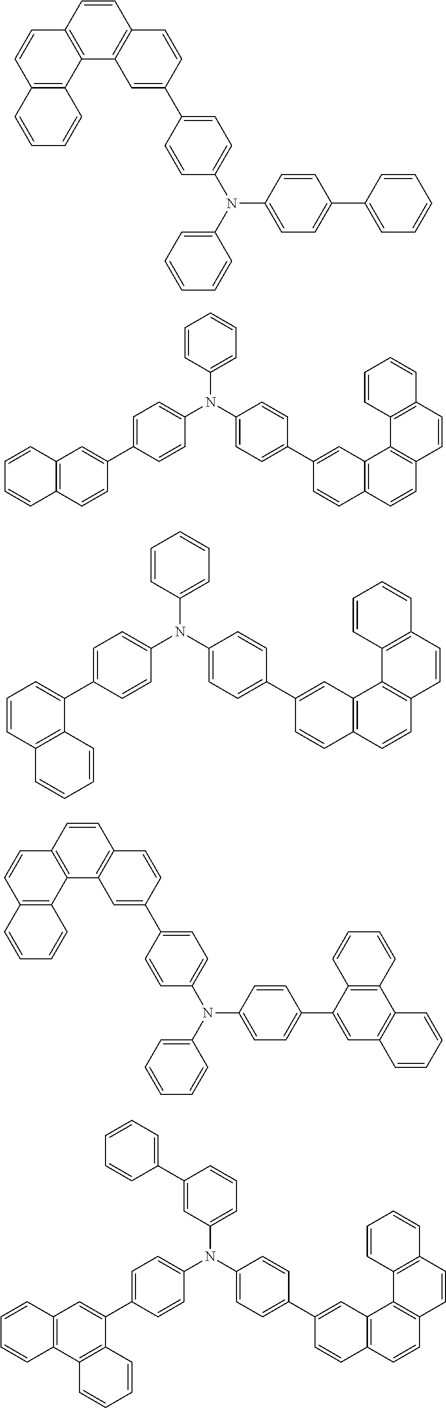

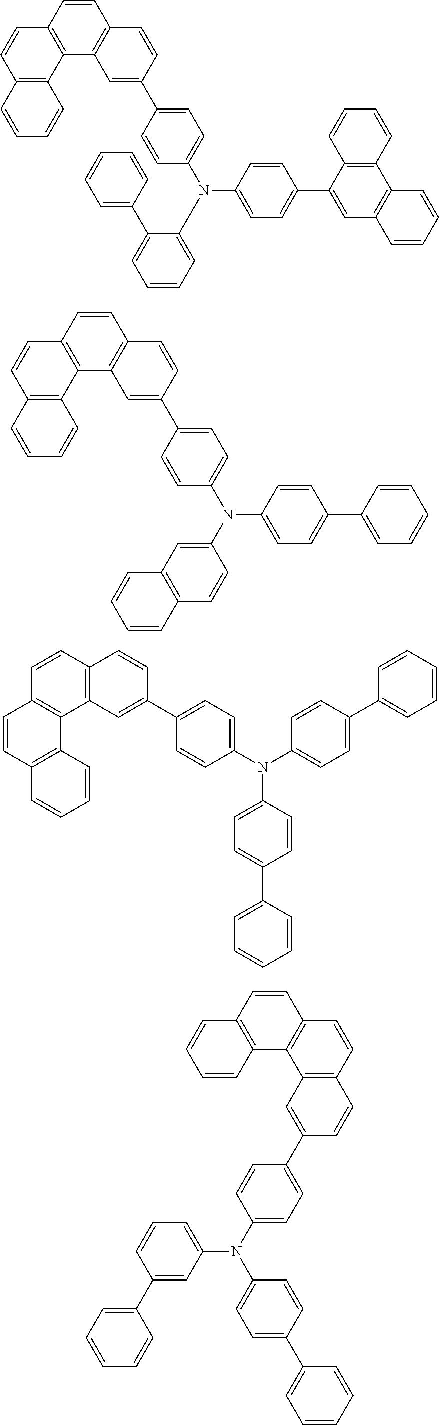

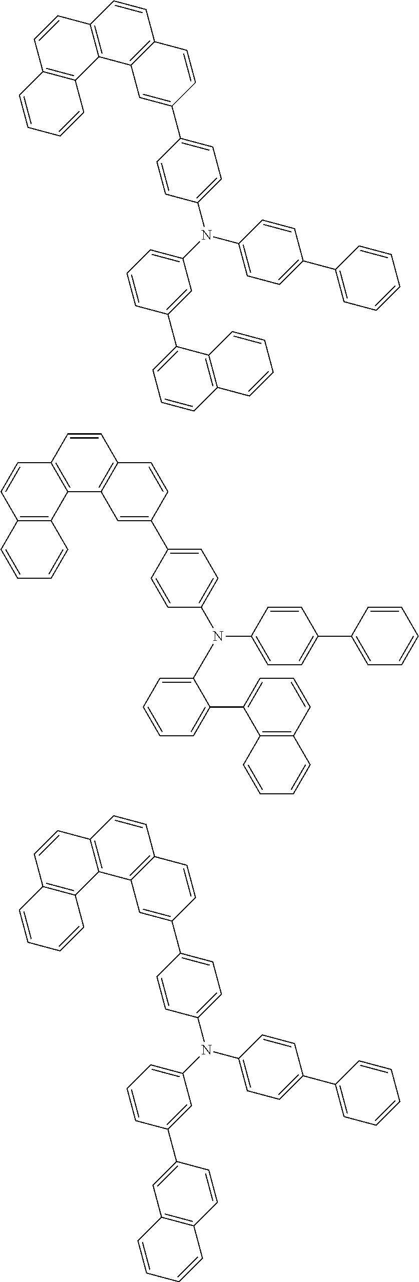 [SMILES: c1ccc(-c2ccc(N(c3ccc(-c4ccc5ccc6ccc7ccccc7c6c5c4)cc3)c3cccc(-c4cccc5ccccc45)c3)cc2)cc1 ; c1ccc(-c2ccc(N(c3ccc(-c4ccc5ccc6ccc7ccccc7c6c5c4)cc3)c3ccccc3-c3cccc4ccccc34)cc2)cc1 ; c1ccc(-c2ccc(N(c3ccc(-c4ccc5ccc6ccc7ccccc7c6c5c4)cc3)c3cccc(-c4ccc5ccccc5c4)c3)cc2)cc1]

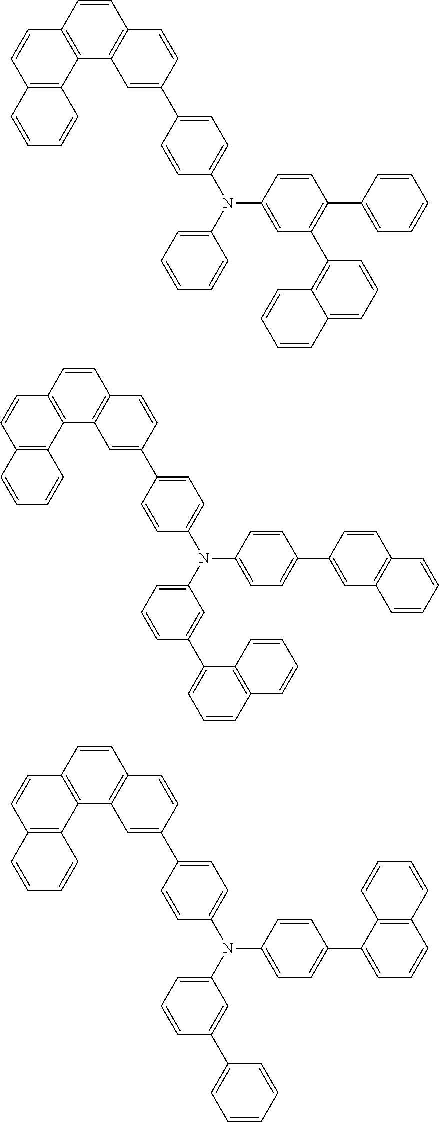 [SMILES: c1ccc(-c2ccc(N(c3ccccc3)c3ccc(-c4ccc5ccc6ccc7ccccc7c6c5c4)cc3)cc2-c2cccc3ccccc23)cc1 ; c1cc(-c2cccc3ccccc23)cc(N(c2ccc(-c3ccc4ccccc4c3)cc2)c2ccc(-c3ccc4ccc5ccc6ccccc6c5c4c3)cc2)c1 ; c1ccc(-c2cccc(N(c3ccc(-c4ccc5ccc6ccc7ccccc7c6c5c4)cc3)c3ccc(-c4cccc5ccccc45)cc3)c2)cc1]

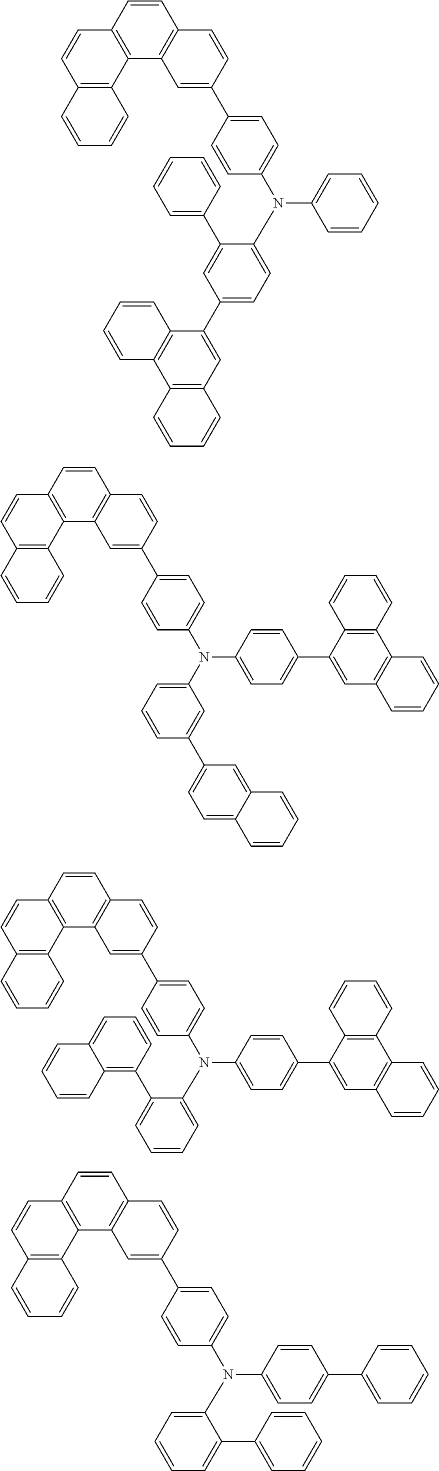 [SMILES: c1ccc(-c2cc(-c3cc4ccccc4c4ccccc34)ccc2N(c2ccccc2)c2ccc(-c3ccc4ccc5ccc6ccccc6c5c4c3)cc2)cc1 ; c1cc(-c2ccc3ccccc3c2)cc(N(c2ccc(-c3ccc4ccc5ccc6ccccc6c5c4c3)cc2)c2ccc(-c3cc4ccccc4c4ccccc34)cc2)c1 ; c1ccc(N(c2ccc(-c3ccc4ccc5ccc6ccccc6c5c4c3)cc2)c2ccc(-c3cc4ccccc4c4ccccc34)cc2)c(-c2cccc3ccccc23)c1 ; c1ccc(-c2ccc(N(c3ccc(-c4ccc5ccc6ccc7ccccc7c6c5c4)cc3)c3ccccc3-c3ccccc3)cc2)cc1]

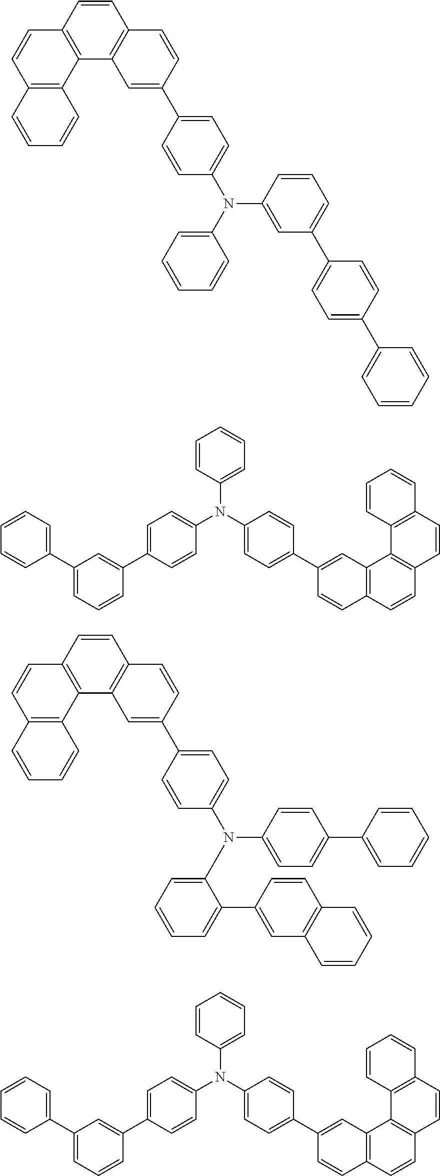

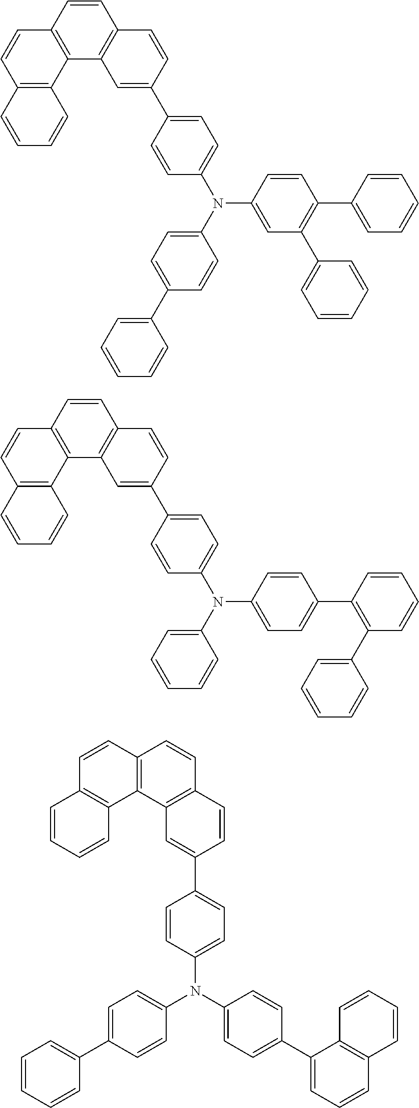

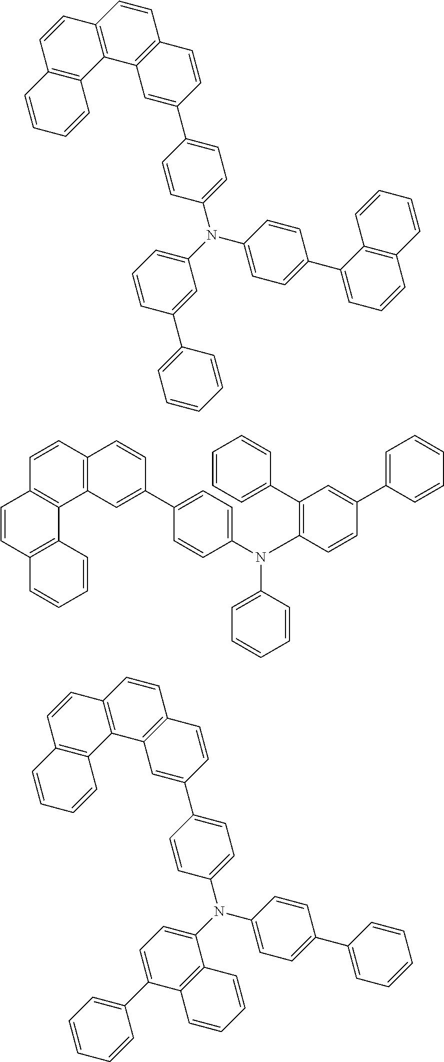

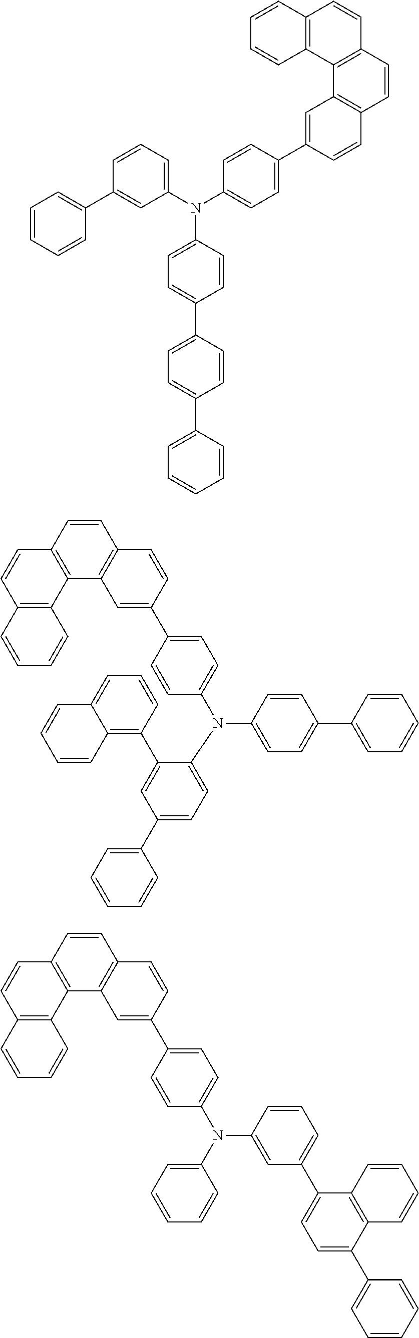

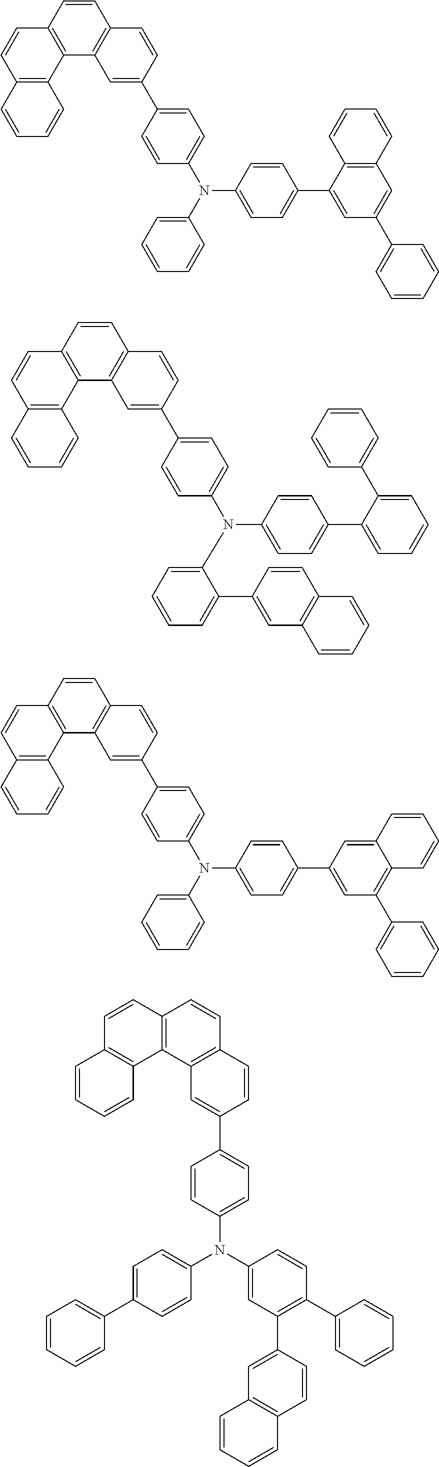 [SMILES: c1ccc(-c2cc(-c3ccc(N(c4ccccc4)c4ccc(-c5ccc6ccc7ccc8ccccc8c7c6c5)cc4)cc3)c3ccccc3c2)cc1 ; c1ccc(-c2ccccc2-c2ccc(N(c3ccc(-c4ccc5ccc6ccc7ccccc7c6c5c4)cc3)c3ccccc3-c3ccc4ccccc4c3)cc2)cc1 ; c1ccc(-c2cc(-c3ccc(N(c4ccccc4)c4ccc(-c5ccc6ccc7ccc8ccccc8c7c6c5)cc4)cc3)cc3ccccc23)cc1 ; c1ccc(-c2ccc(N(c3ccc(-c4ccc5ccc6ccc7ccccc7c6c5c4)cc3)c3ccc(-c4ccccc4)c(-c4ccc5ccccc5c4)c3)cc2)cc1]

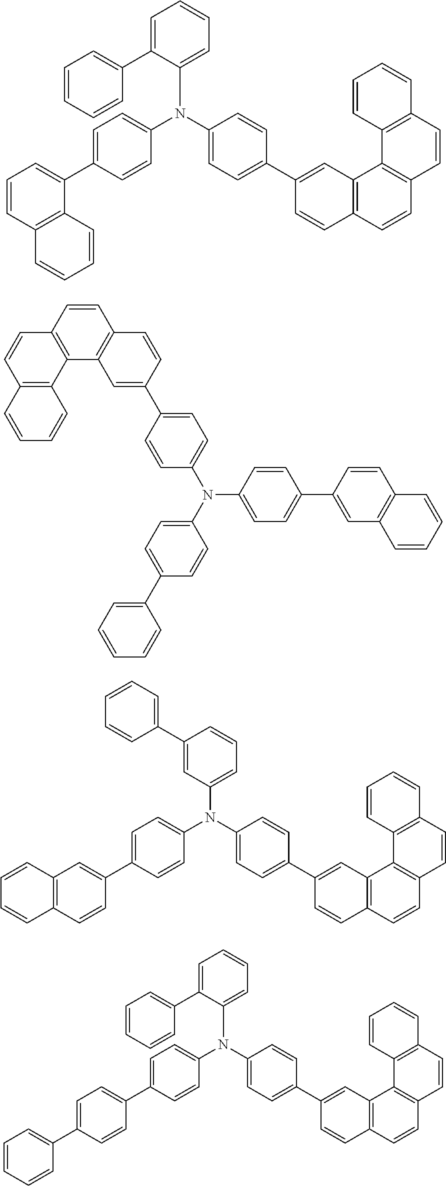

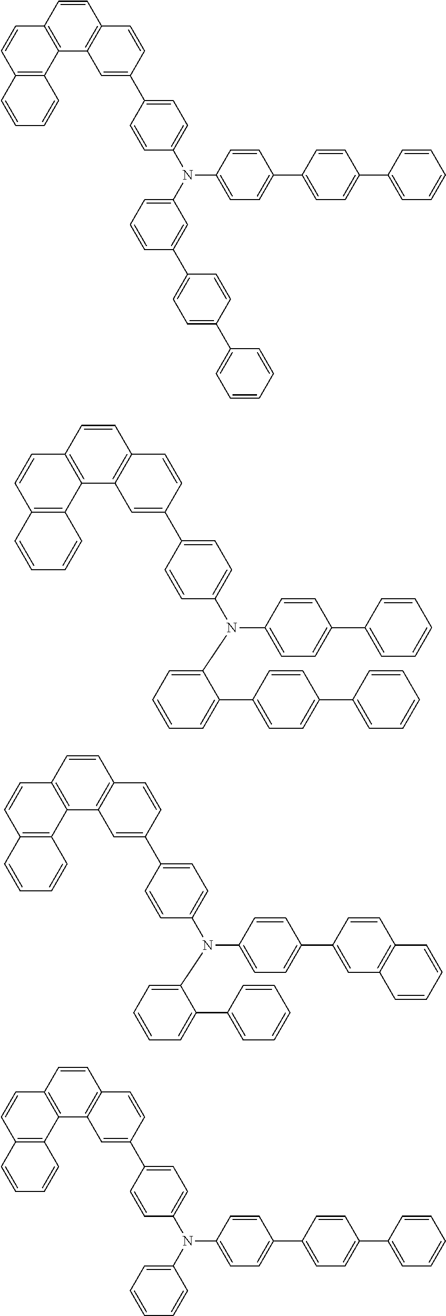 [SMILES: c1ccc(-c2ccc(-c3ccc(N(c4ccc(-c5ccc6ccc7ccc8ccccc8c7c6c5)cc4)c4cccc(-c5ccc(-c6ccccc6)cc5)c4)cc3)cc2)cc1 ; c1ccc(-c2ccc(-c3ccccc3N(c3ccc(-c4ccccc4)cc3)c3ccc(-c4ccc5ccc6ccc7ccccc7c6c5c4)cc3)cc2)cc1 ; c1ccc(-c2ccccc2N(c2ccc(-c3ccc4ccccc4c3)cc2)c2ccc(-c3ccc4ccc5ccc6ccccc6c5c4c3)cc2)cc1 ; c1ccc(-c2ccc(-c3ccc(N(c4ccccc4)c4ccc(-c5ccc6ccc7ccc8ccccc8c7c6c5)cc4)cc3)cc2)cc1]

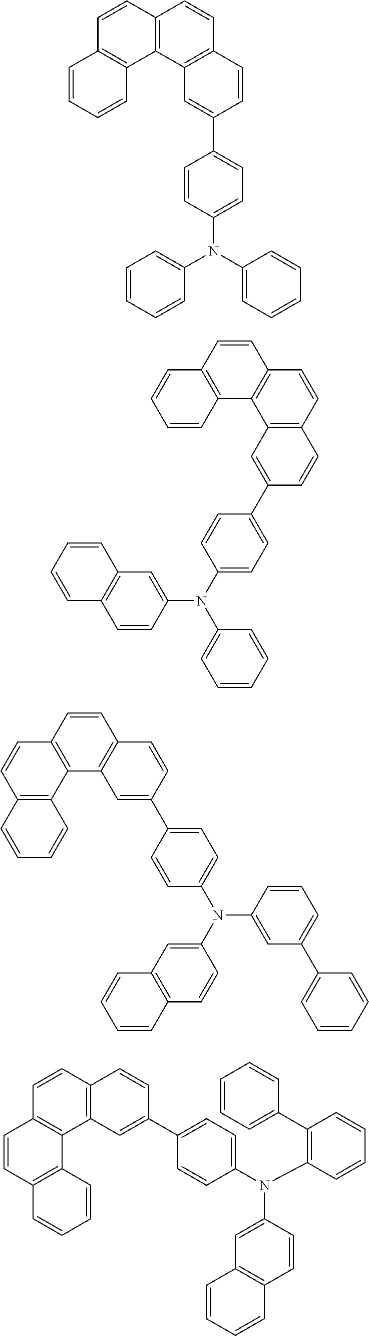

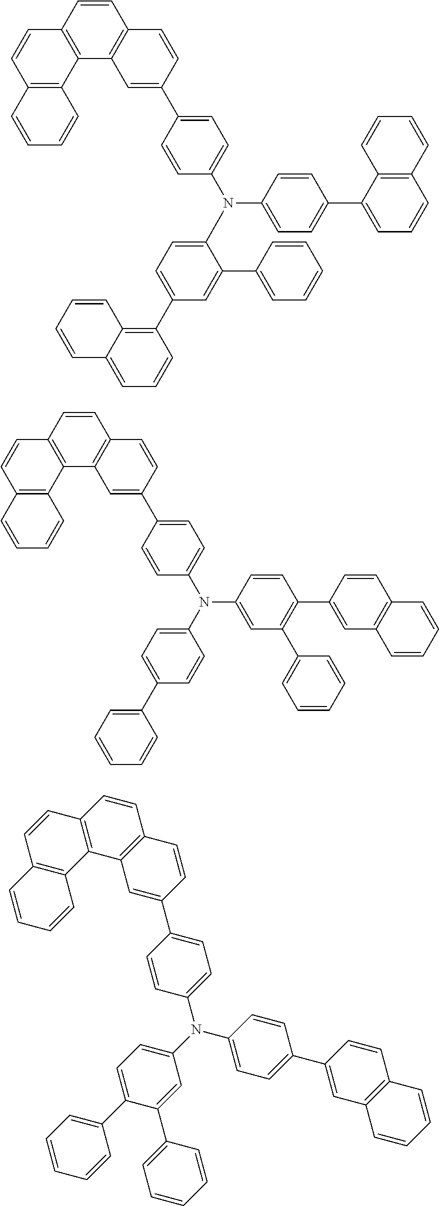

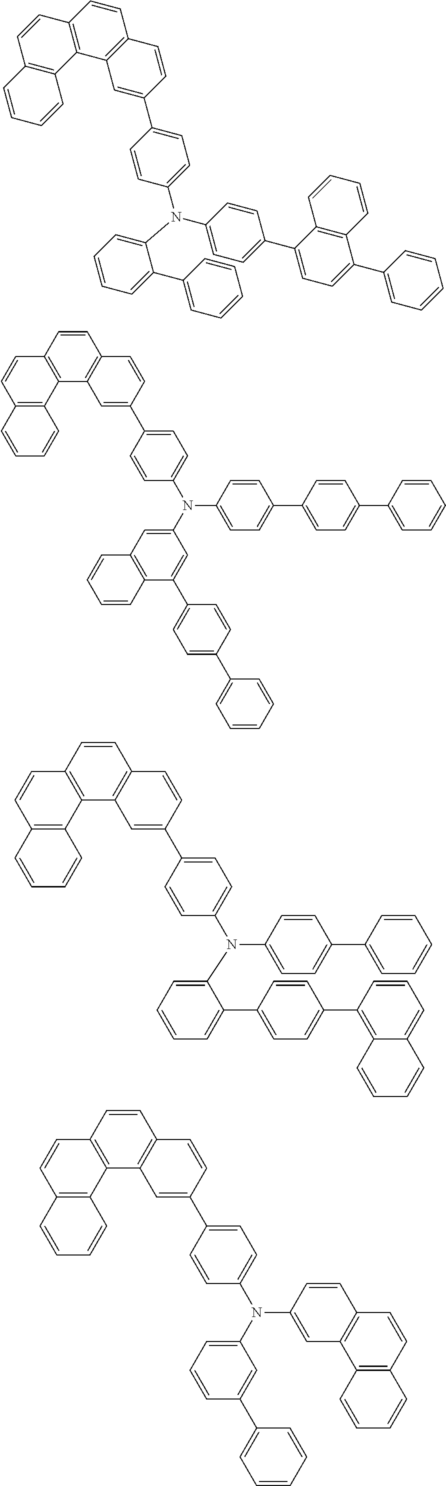 [SMILES: c1ccc(-c2ccccc2N(c2ccc(-c3ccc4ccc5ccc6ccccc6c5c4c3)cc2)c2ccc(-c3ccc(-c4ccccc4)c4ccccc34)cc2)cc1 ; c1ccc(-c2ccc(-c3ccc(N(c4ccc(-c5ccc6ccc7ccc8ccccc8c7c6c5)cc4)c4cc(-c5ccc(-c6ccccc6)cc5)c5ccccc5c4)cc3)cc2)cc1 ; c1ccc(-c2ccc(N(c3ccc(-c4ccc5ccc6ccc7ccccc7c6c5c4)cc3)c3ccccc3-c3ccc(-c4cccc5ccccc45)cc3)cc2)cc1 ; c1ccc(-c2cccc(N(c3ccc(-c4ccc5ccc6ccc7ccccc7c6c5c4)cc3)c3ccc4ccc5ccccc5c4c3)c2)cc1]

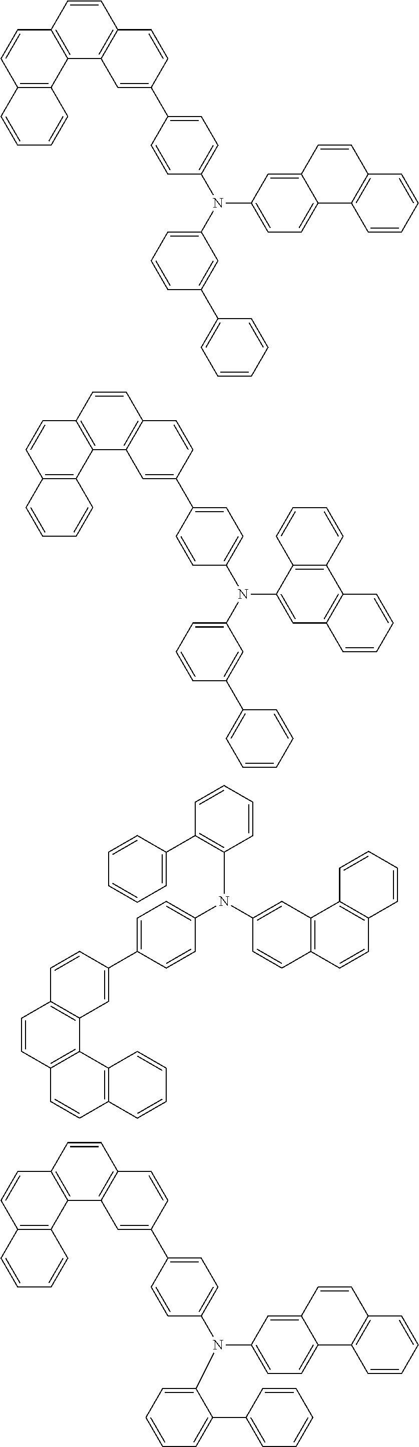

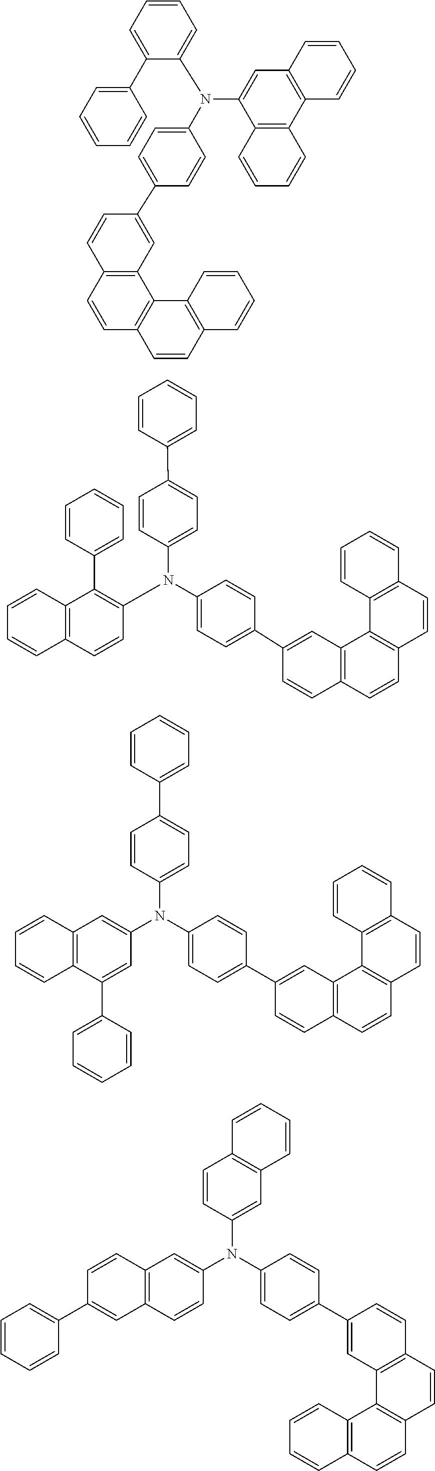

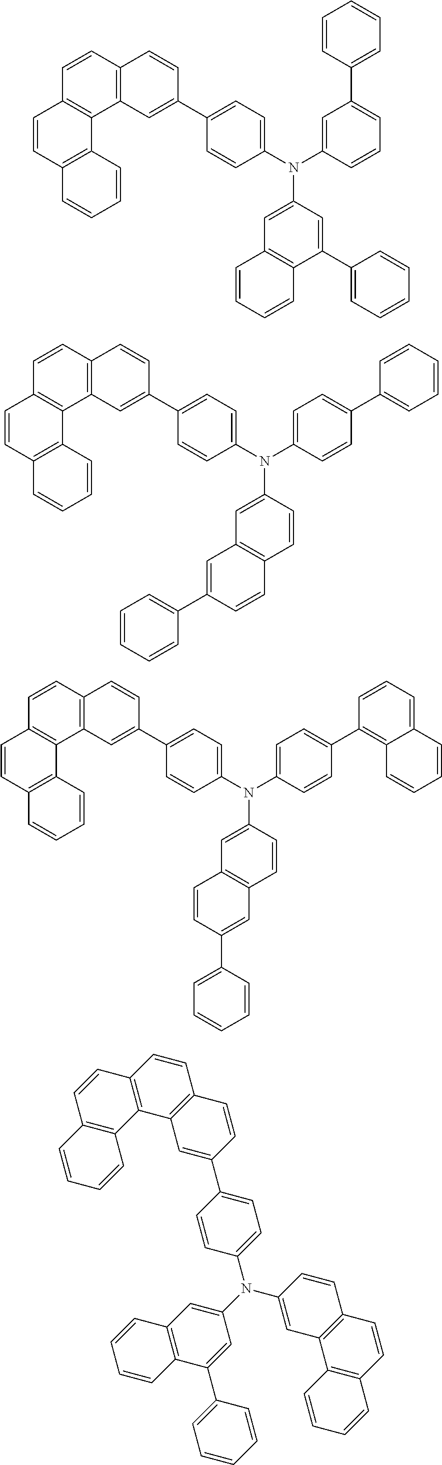

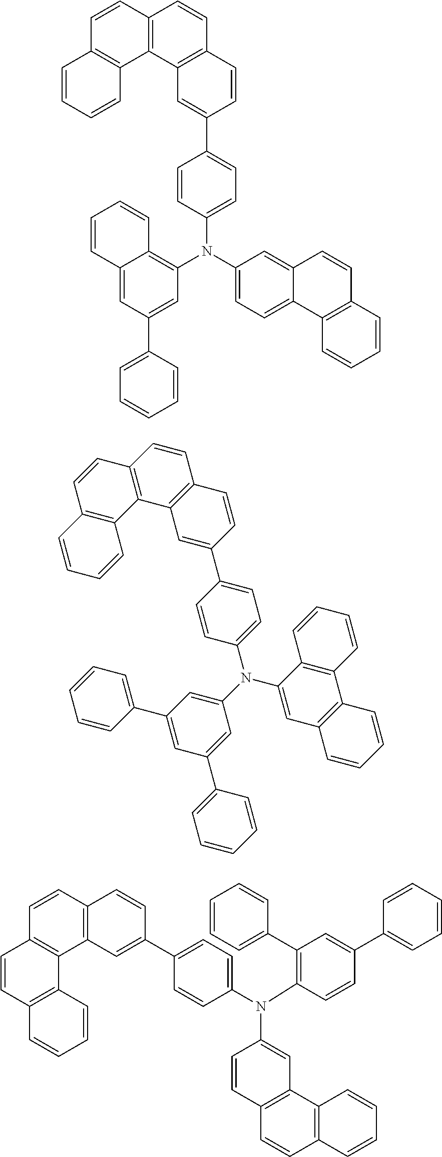

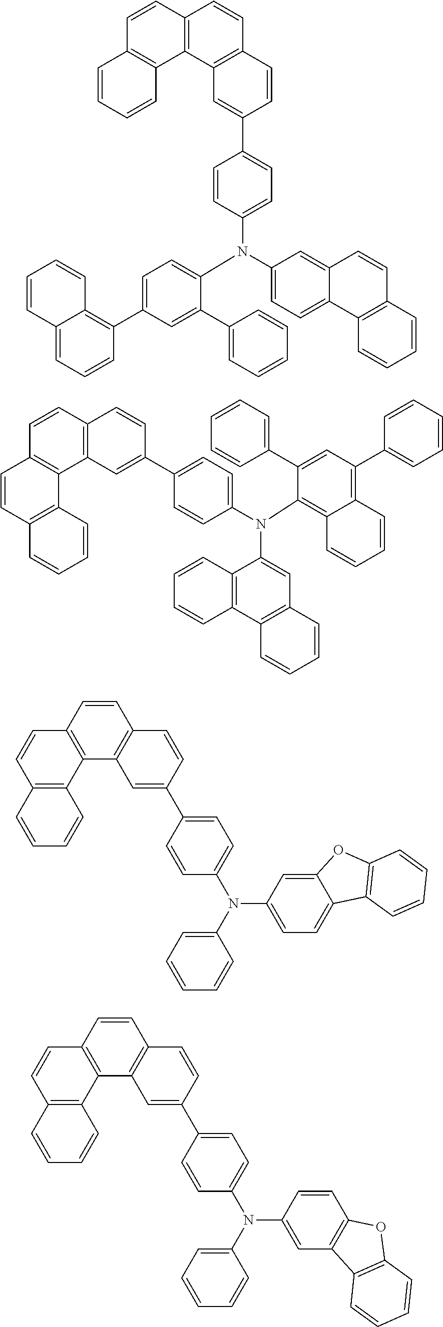

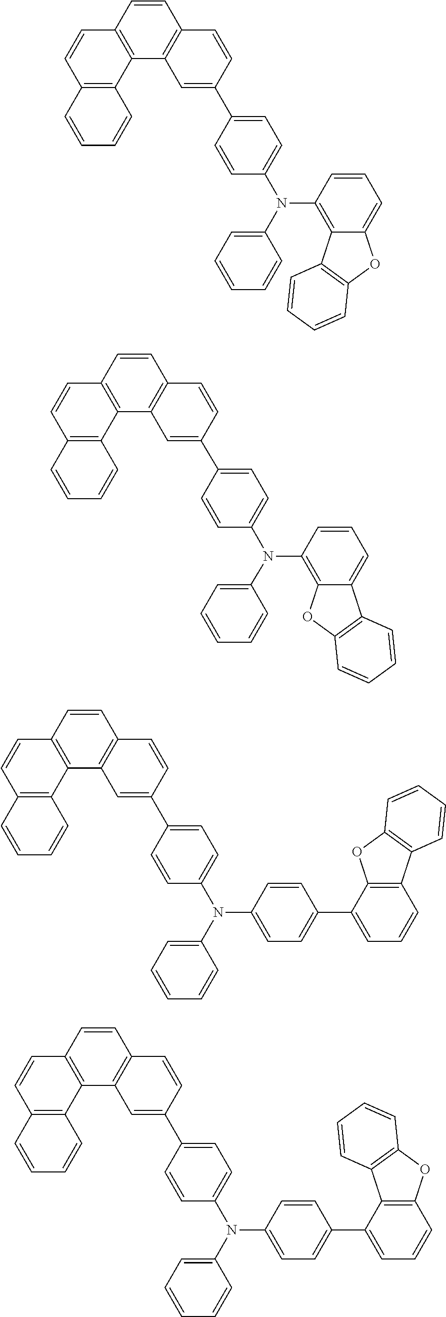 [SMILES: c1ccc(N(c2ccc(-c3ccc4ccc5ccc6ccccc6c5c4c3)cc2)c2cccc3oc4ccccc4c23)cc1 ; c1ccc(N(c2ccc(-c3ccc4ccc5ccc6ccccc6c5c4c3)cc2)c2cccc3c2oc2ccccc23)cc1 ; c1ccc(N(c2ccc(-c3ccc4ccc5ccc6ccccc6c5c4c3)cc2)c2ccc(-c3cccc4c3oc3ccccc34)cc2)cc1 ; c1ccc(N(c2ccc(-c3ccc4ccc5ccc6ccccc6c5c4c3)cc2)c2ccc(-c3cccc4oc5ccccc5c34)cc2)cc1]

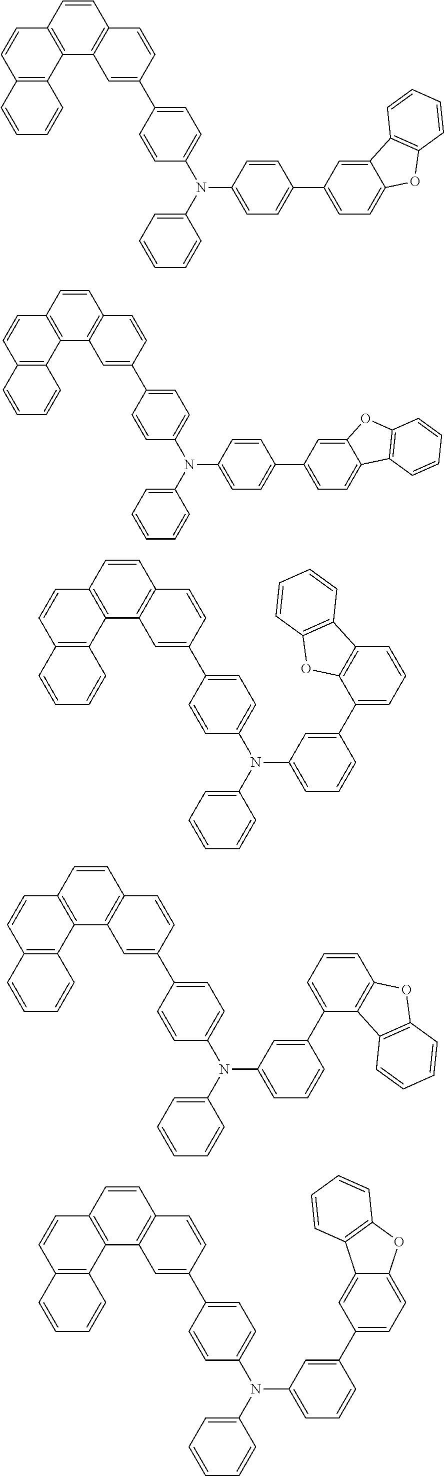

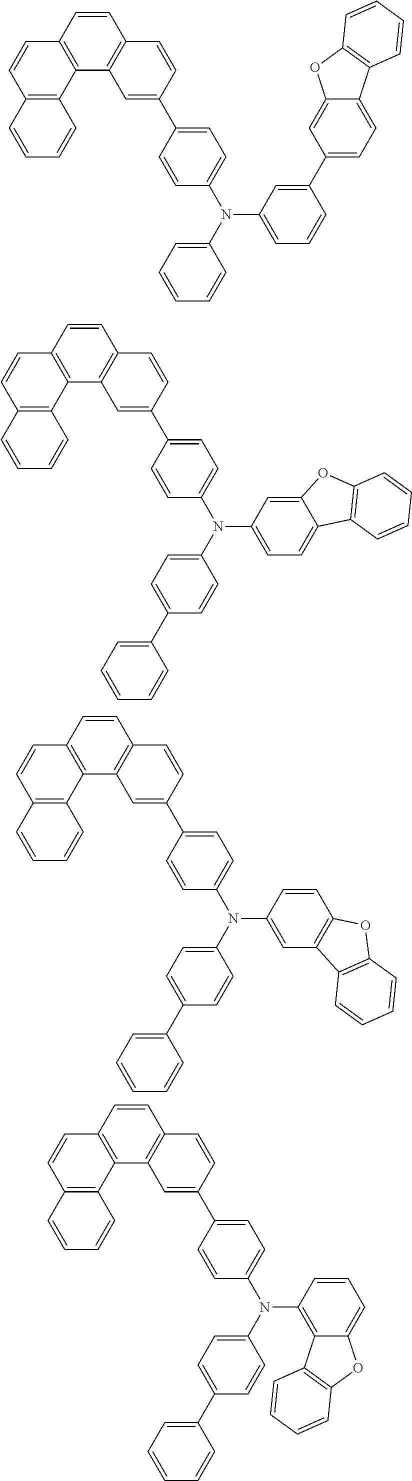

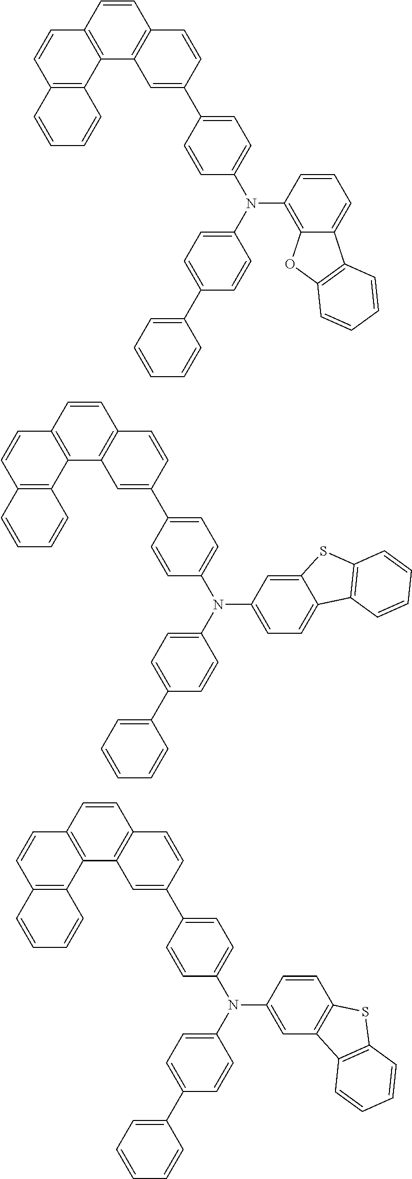

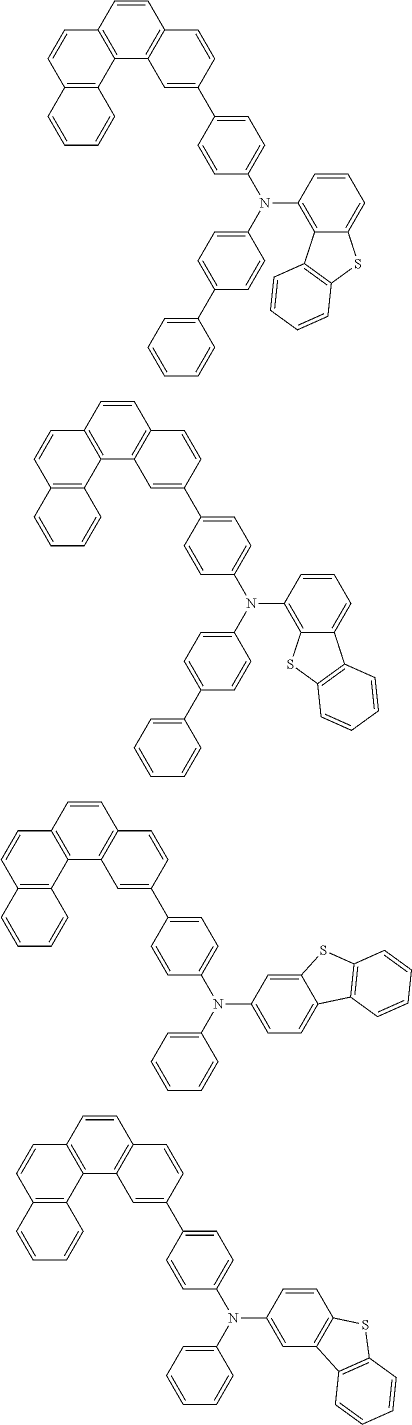

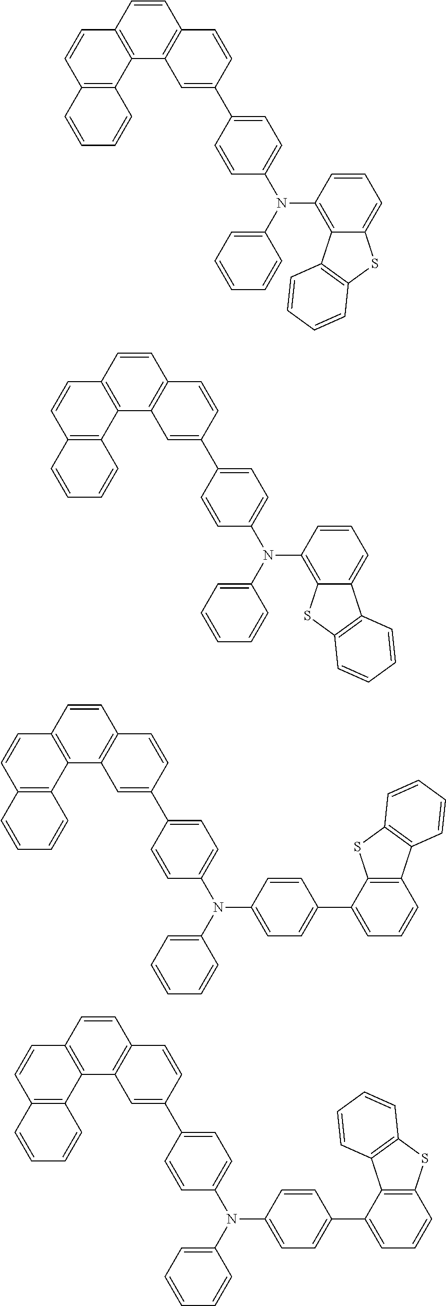 [SMILES: c1ccc(N(c2ccc(-c3ccc4ccc5ccc6ccccc6c5c4c3)cc2)c2cccc3sc4ccccc4c23)cc1 ; c1ccc(N(c2ccc(-c3ccc4ccc5ccc6ccccc6c5c4c3)cc2)c2cccc3c2sc2ccccc23)cc1 ; c1ccc(N(c2ccc(-c3ccc4ccc5ccc6ccccc6c5c4c3)cc2)c2ccc(-c3cccc4c3sc3ccccc34)cc2)cc1 ; c1ccc(N(c2ccc(-c3ccc4ccc5ccc6ccccc6c5c4c3)cc2)c2ccc(-c3cccc4sc5ccccc5c34)cc2)cc1]

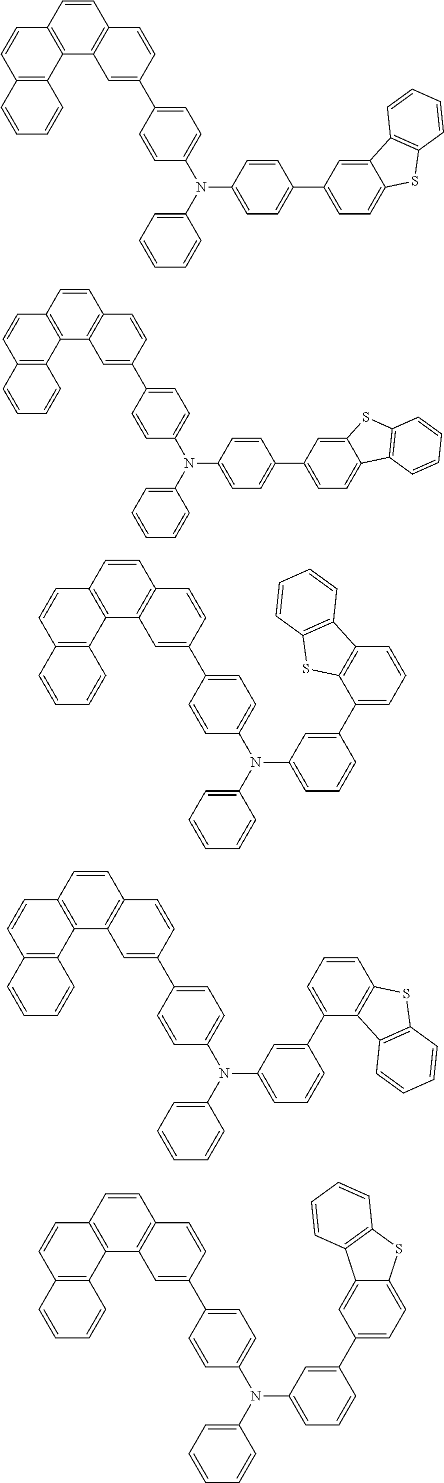

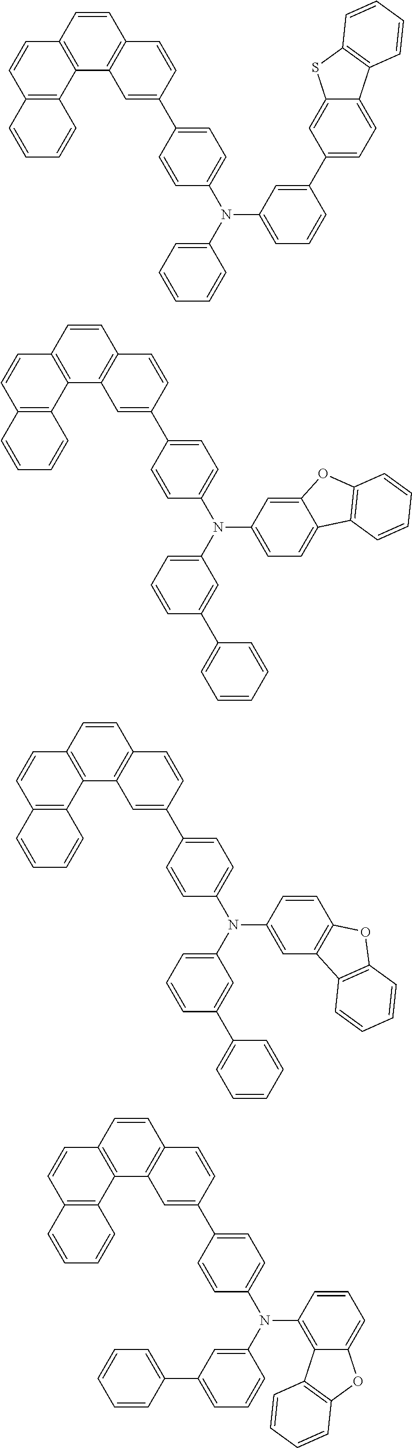 [SMILES: c1ccc(N(c2ccc(-c3ccc4ccc5ccc6ccccc6c5c4c3)cc2)c2cccc(-c3ccc4c(c3)sc3ccccc34)c2)cc1 ; c1ccc(-c2cccc(N(c3ccc(-c4ccc5ccc6ccc7ccccc7c6c5c4)cc3)c3ccc4c(c3)oc3ccccc34)c2)cc1 ; c1ccc(-c2cccc(N(c3ccc(-c4ccc5ccc6ccc7ccccc7c6c5c4)cc3)c3ccc4oc5ccccc5c4c3)c2)cc1 ; c1ccc(-c2cccc(N(c3ccc(-c4ccc5ccc6ccc7ccccc7c6c5c4)cc3)c3cccc4oc5ccccc5c34)c2)cc1]

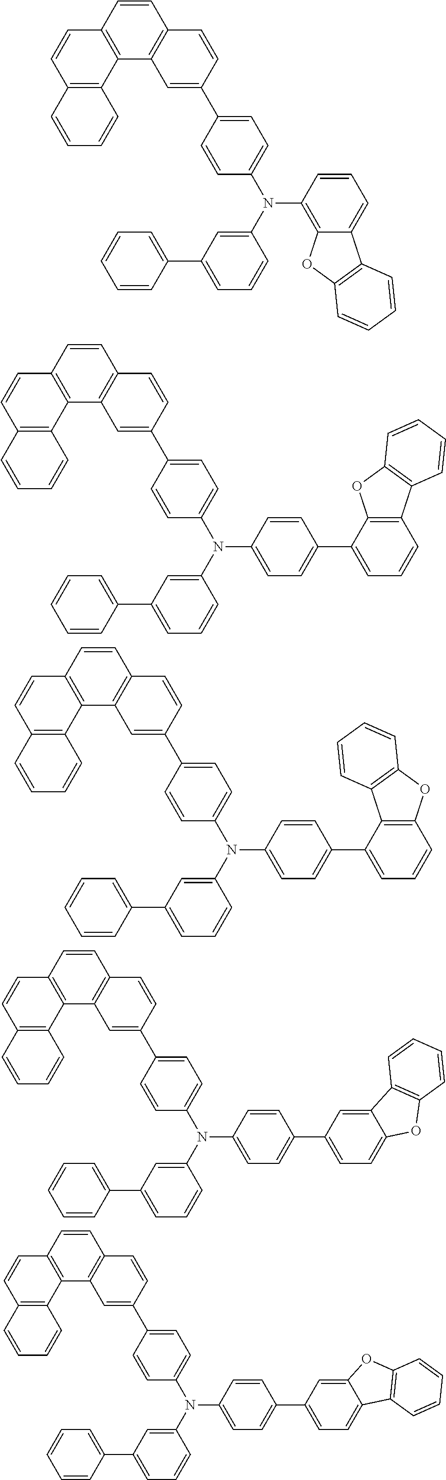

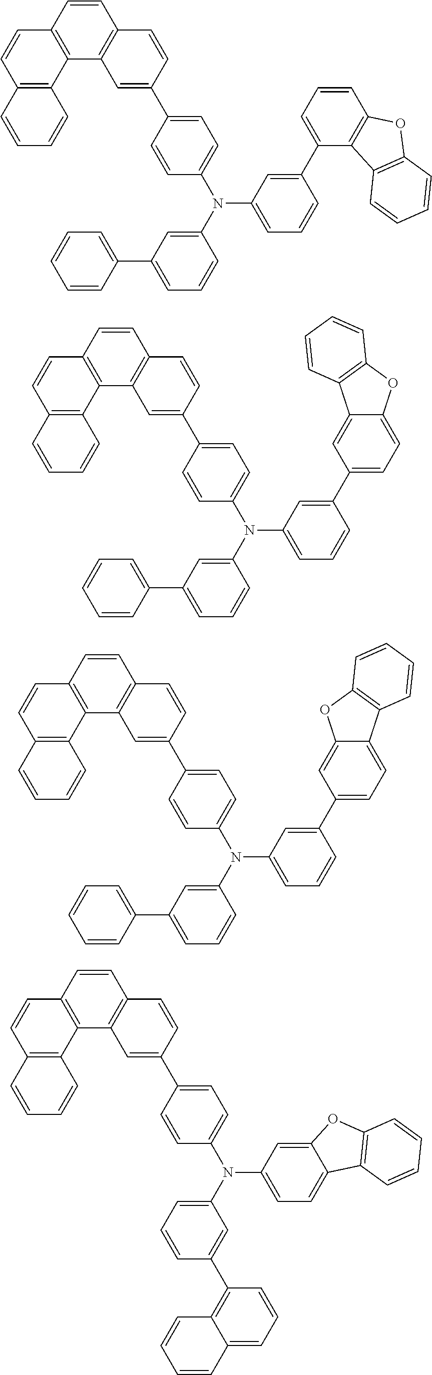

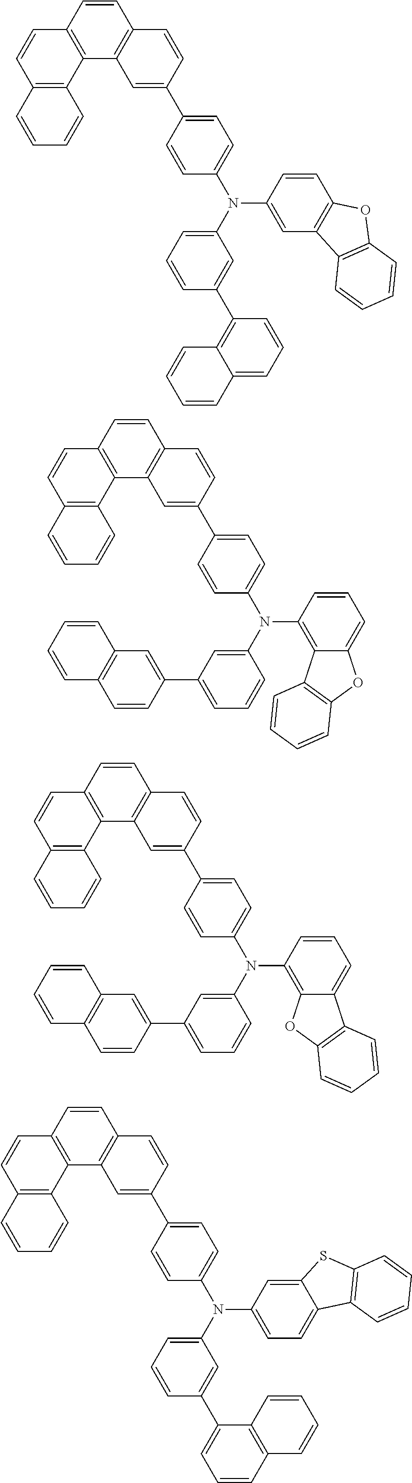

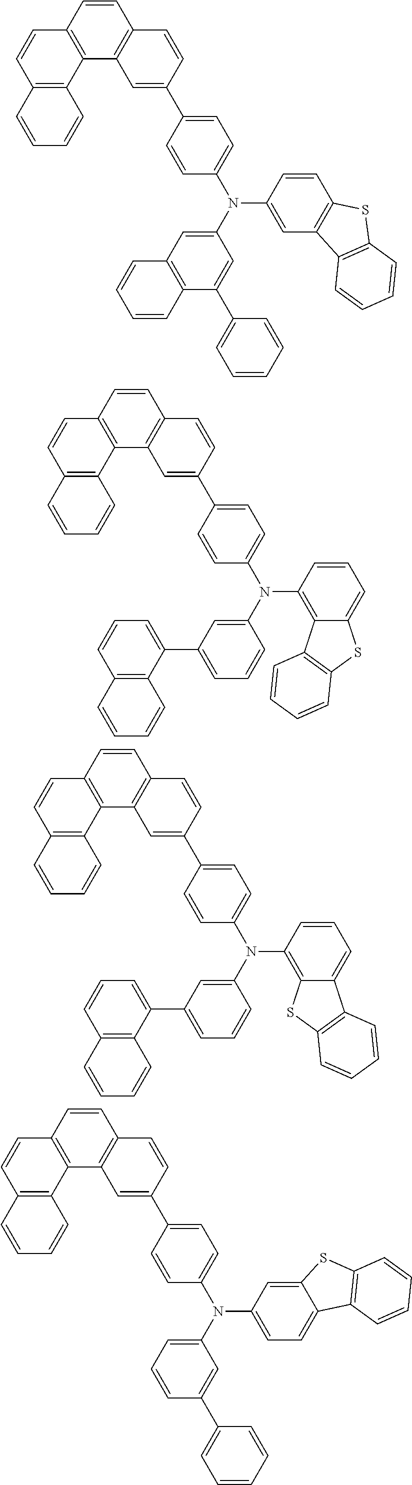

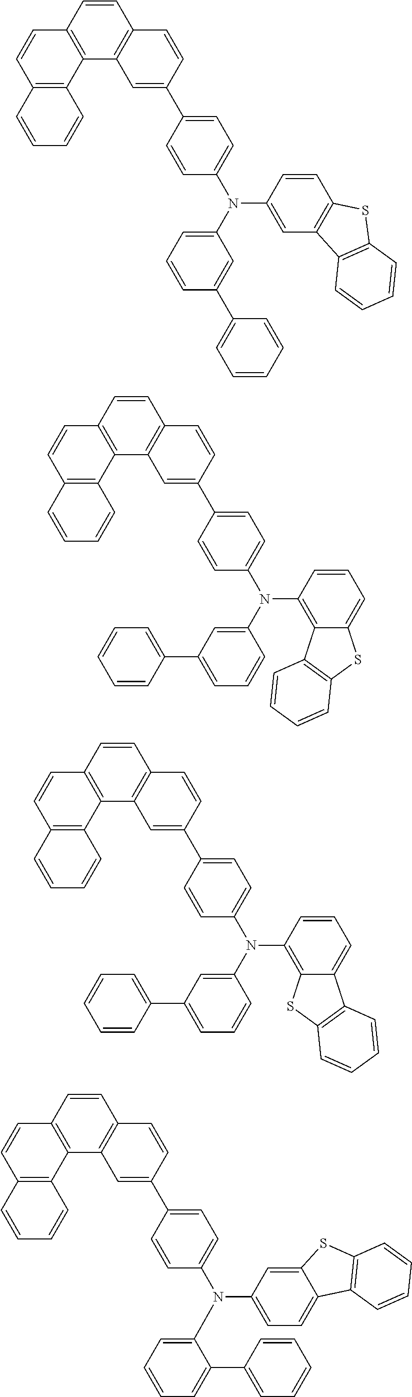 [SMILES: c1ccc(-c2cccc(N(c3ccc(-c4ccc5ccc6ccc7ccccc7c6c5c4)cc3)c3ccc4sc5ccccc5c4c3)c2)cc1 ; c1ccc(-c2cccc(N(c3ccc(-c4ccc5ccc6ccc7ccccc7c6c5c4)cc3)c3cccc4sc5ccccc5c34)c2)cc1 ; c1ccc(-c2cccc(N(c3ccc(-c4ccc5ccc6ccc7ccccc7c6c5c4)cc3)c3cccc4c3sc3ccccc34)c2)cc1 ; c1ccc(-c2ccccc2N(c2ccc(-c3ccc4ccc5ccc6ccccc6c5c4c3)cc2)c2ccc3c(c2)sc2ccccc23)cc1]

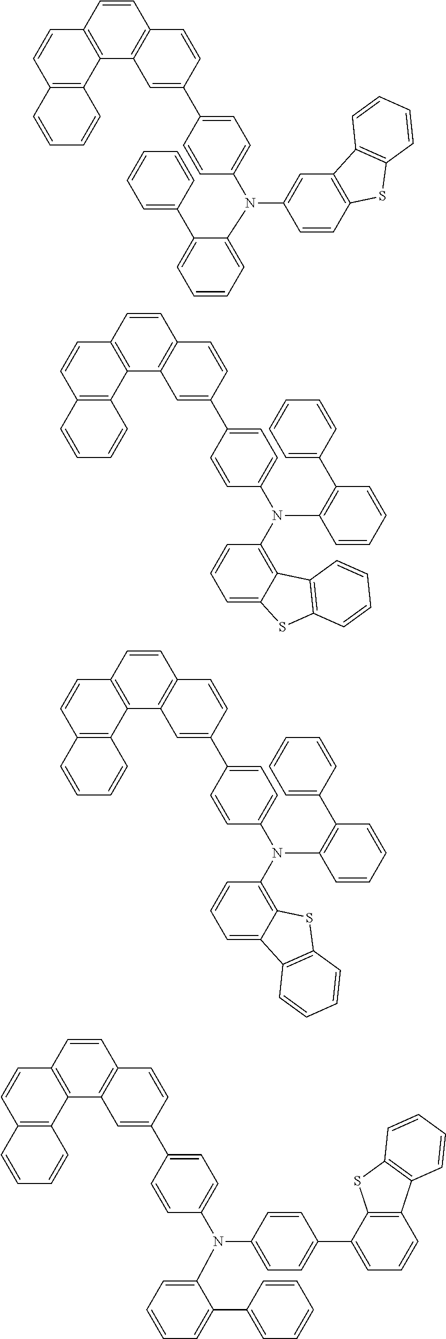

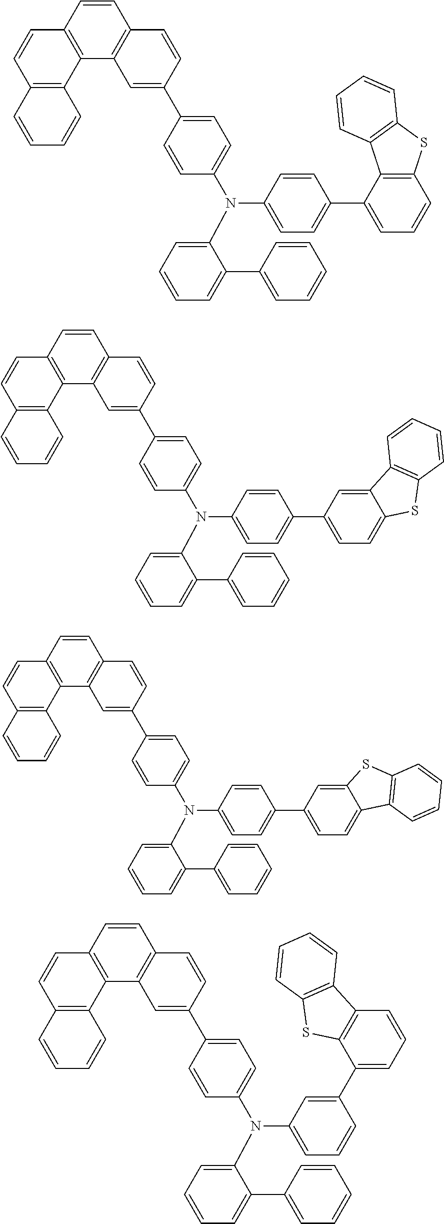

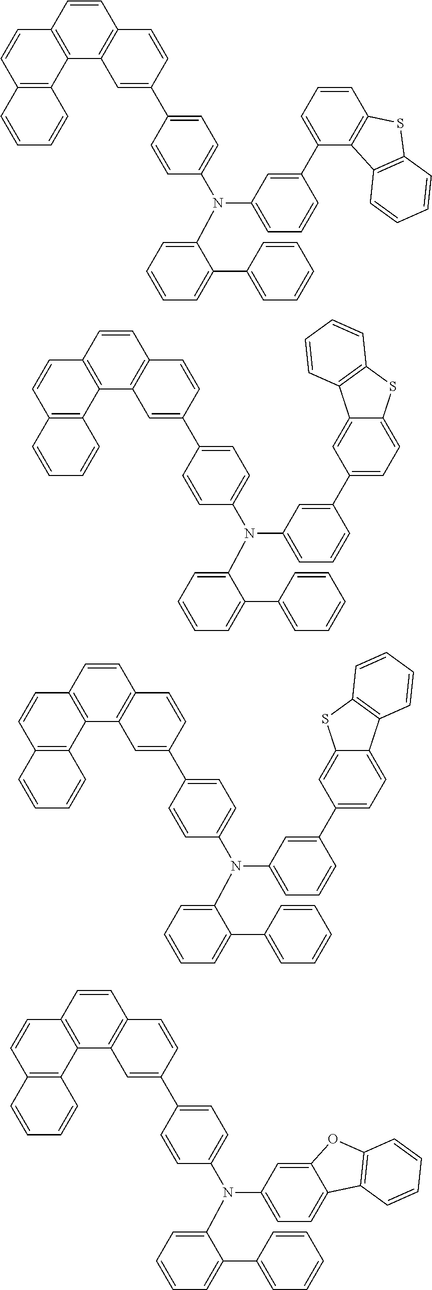

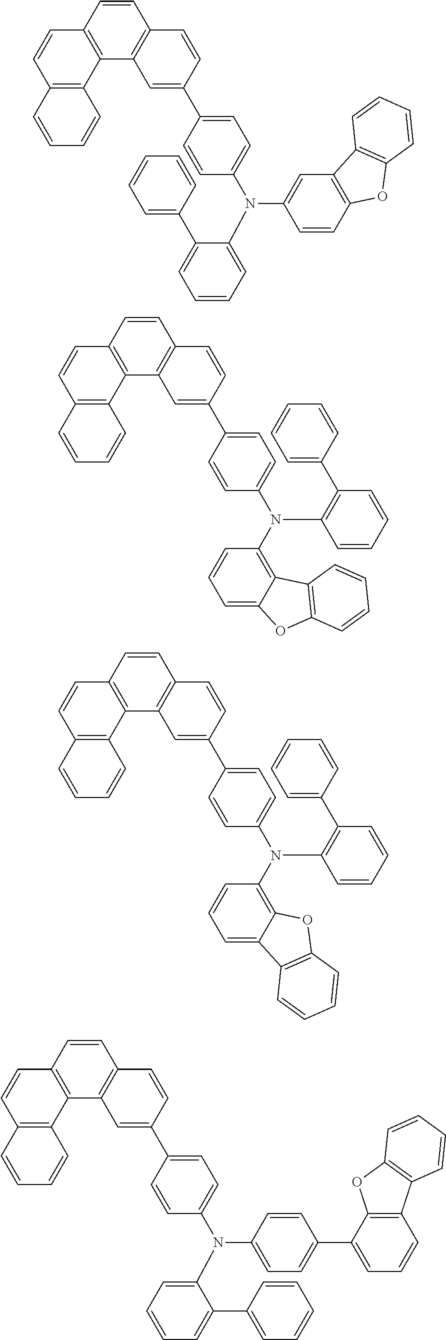

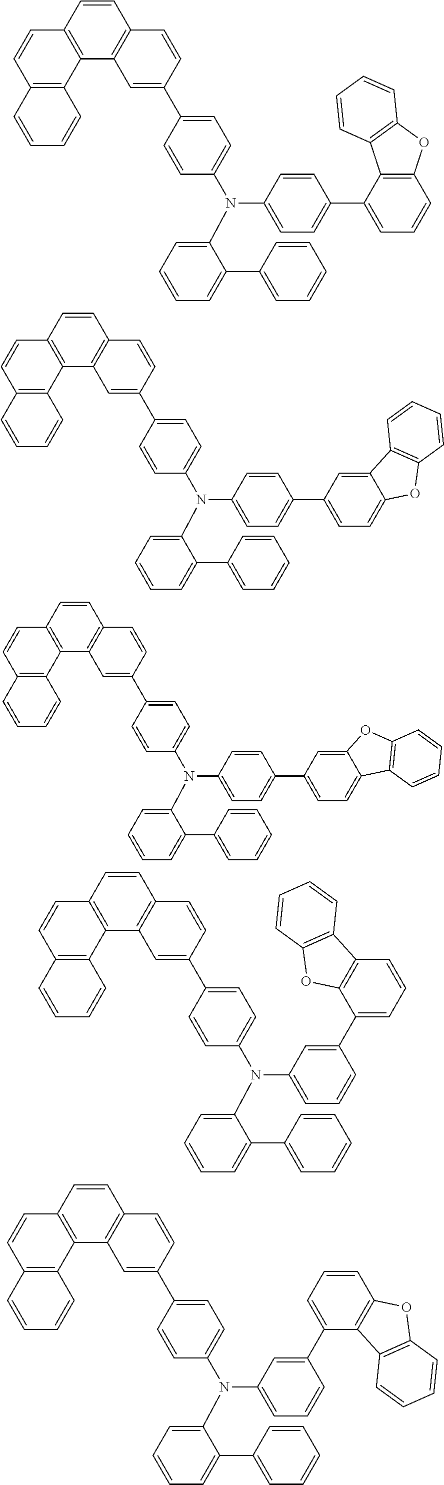

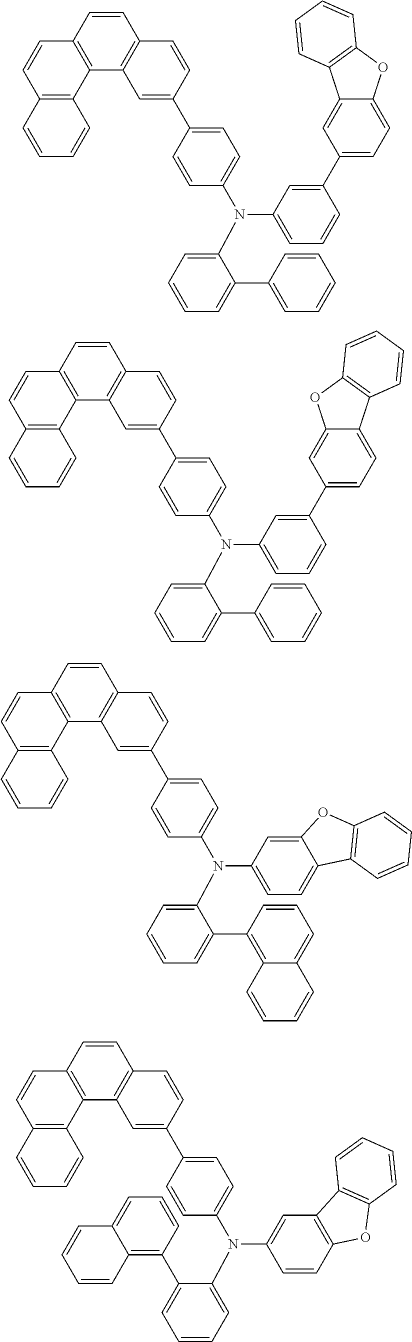

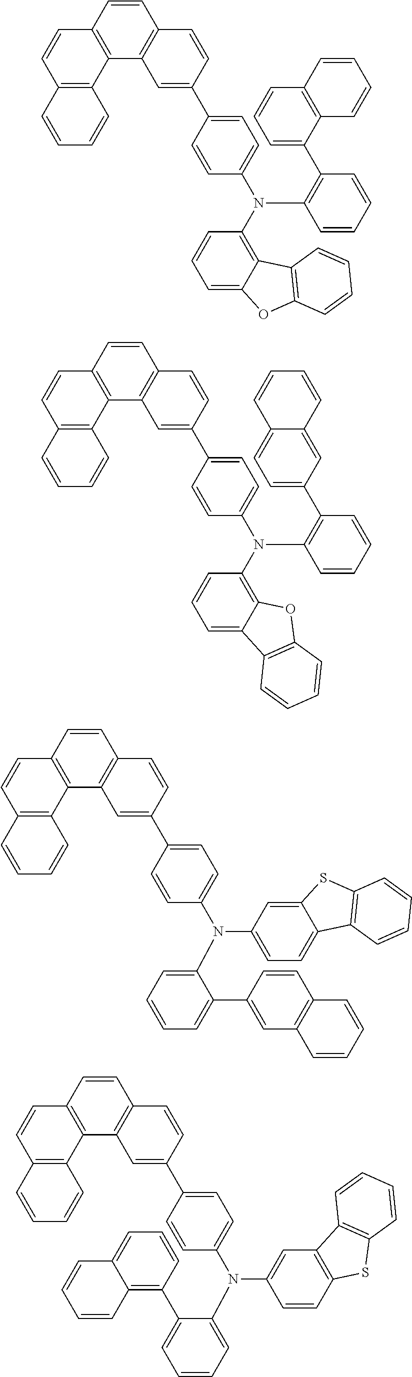 [SMILES: c1ccc(N(c2ccc(-c3ccc4ccc5ccc6ccccc6c5c4c3)cc2)c2cccc3oc4ccccc4c23)c(-c2cccc3ccccc23)c1 ; c1ccc(N(c2ccc(-c3ccc4ccc5ccc6ccccc6c5c4c3)cc2)c2cccc3c2oc2ccccc23)c(-c2ccc3ccccc3c2)c1 ; c1ccc(N(c2ccc(-c3ccc4ccc5ccc6ccccc6c5c4c3)cc2)c2ccc3c(c2)sc2ccccc23)c(-c2ccc3ccccc3c2)c1 ; c1ccc(N(c2ccc(-c3ccc4ccc5ccc6ccccc6c5c4c3)cc2)c2ccc3sc4ccccc4c3c2)c(-c2cccc3ccccc23)c1]

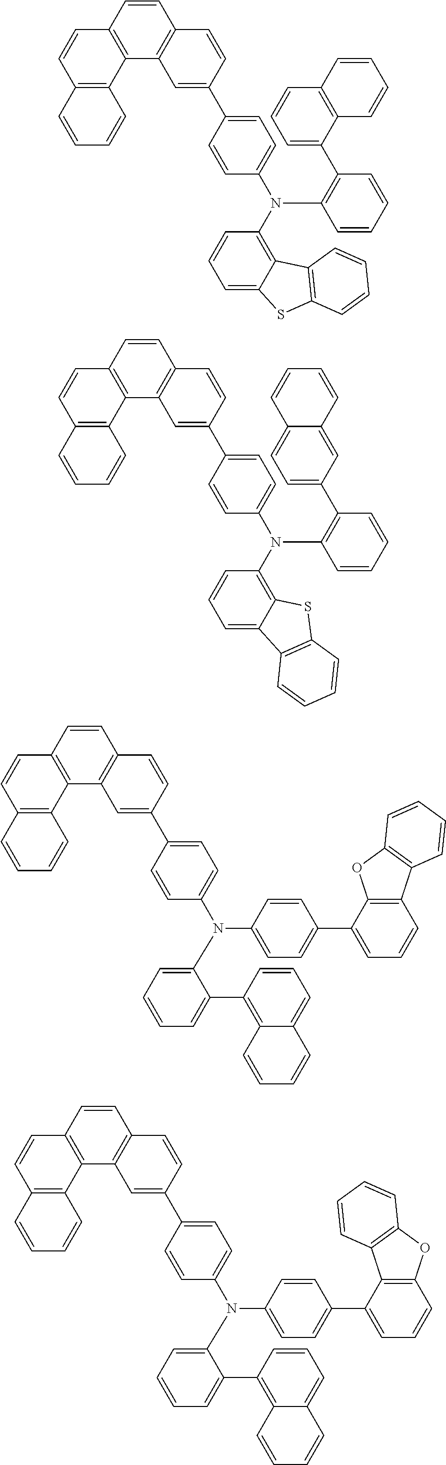 [SMILES: c1ccc(N(c2ccc(-c3ccc4ccc5ccc6ccccc6c5c4c3)cc2)c2cccc3sc4ccccc4c23)c(-c2cccc3ccccc23)c1 ; c1ccc(N(c2ccc(-c3ccc4ccc5ccc6ccccc6c5c4c3)cc2)c2cccc3c2sc2ccccc23)c(-c2ccc3ccccc3c2)c1 ; c1ccc(N(c2ccc(-c3ccc4ccc5ccc6ccccc6c5c4c3)cc2)c2ccc(-c3cccc4c3oc3ccccc34)cc2)c(-c2cccc3ccccc23)c1 ; c1ccc(N(c2ccc(-c3ccc4ccc5ccc6ccccc6c5c4c3)cc2)c2ccc(-c3cccc4oc5ccccc5c34)cc2)c(-c2cccc3ccccc23)c1]

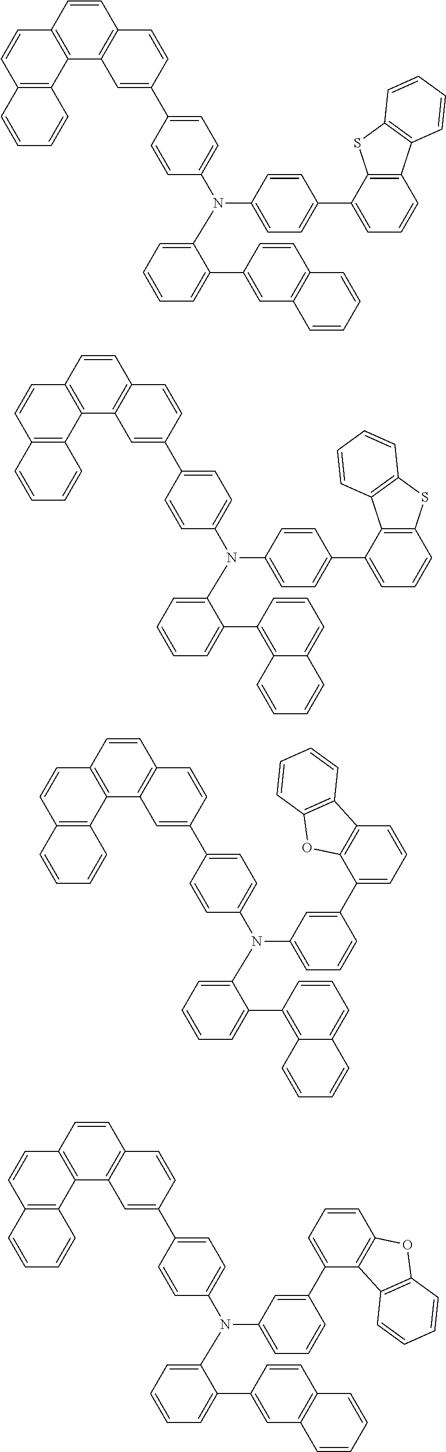

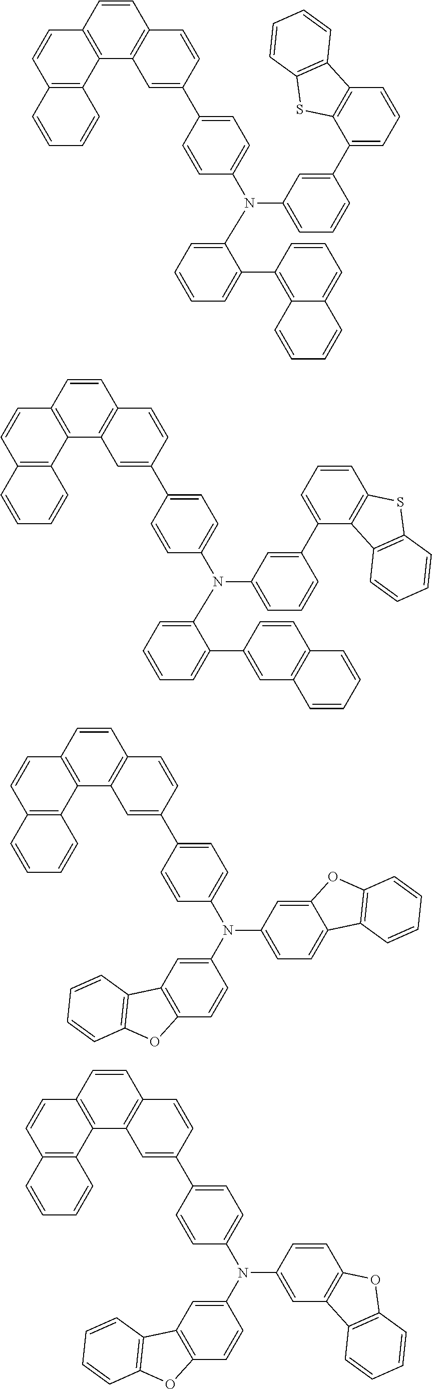

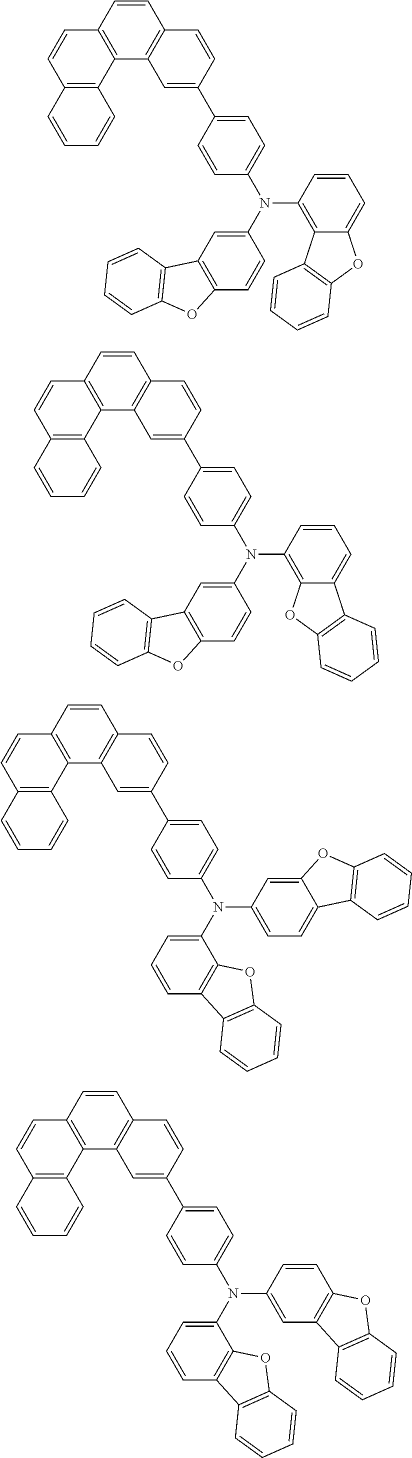

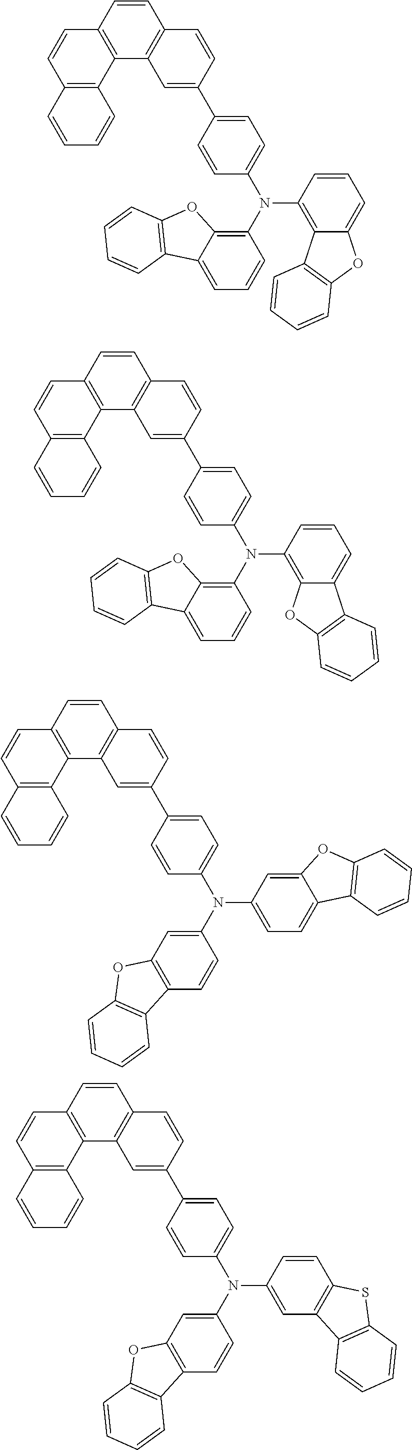

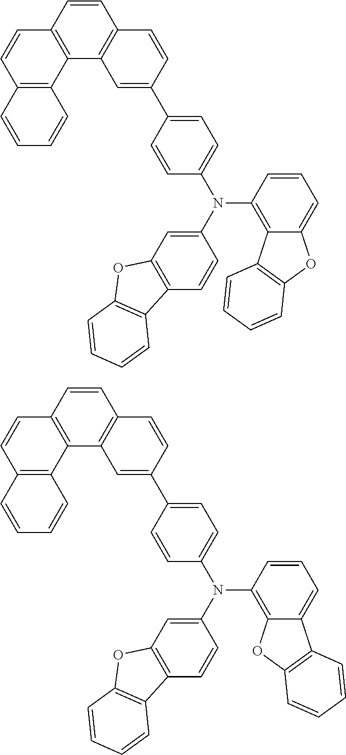

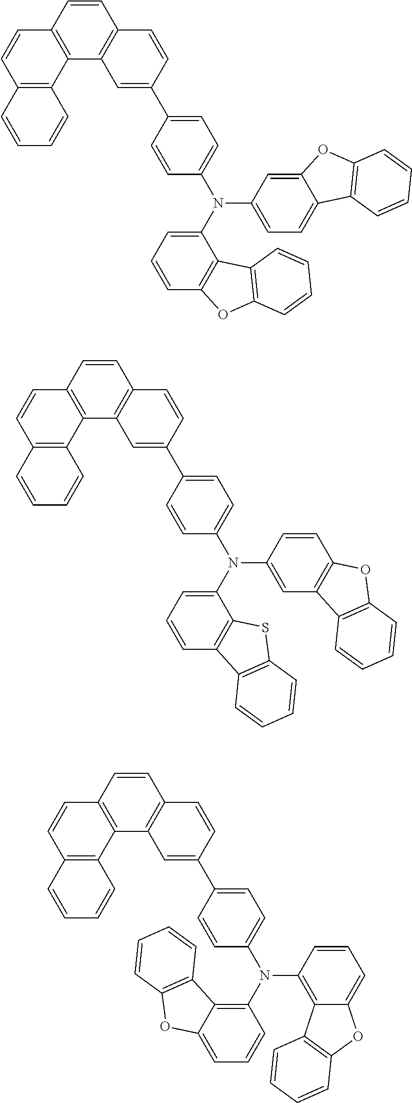 [SMILES: c1ccc2c(c1)ccc1ccc3ccc(-c4ccc(N(c5ccc6c(c5)oc5ccccc56)c5cccc6oc7ccccc7c56)cc4)cc3c12 ; c1ccc2c(c1)ccc1ccc3ccc(-c4ccc(N(c5ccc6oc7ccccc7c6c5)c5cccc6c5sc5ccccc56)cc4)cc3c12 ; c1ccc2c(c1)ccc1ccc3ccc(-c4ccc(N(c5cccc6oc7ccccc7c56)c5cccc6oc7ccccc7c56)cc4)cc3c12]

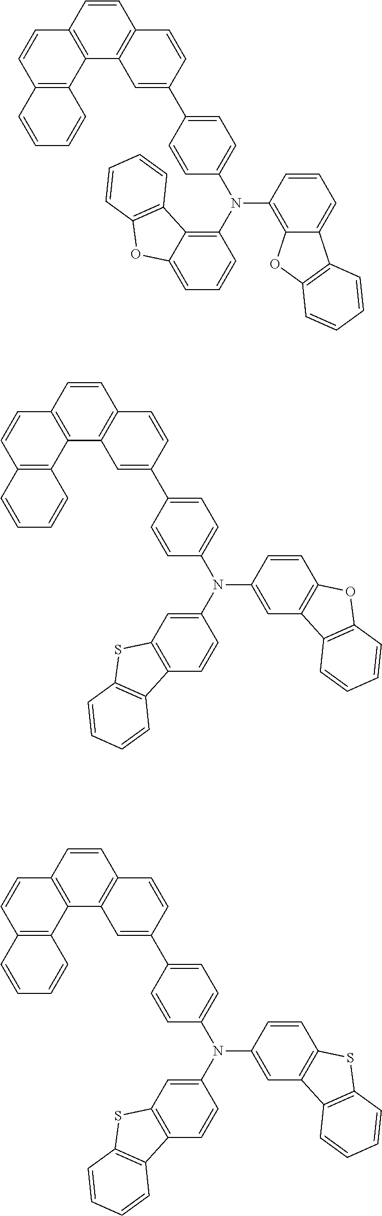 [SMILES: c1ccc2c(c1)ccc1ccc3ccc(-c4ccc(N(c5cccc6c5oc5ccccc56)c5cccc6oc7ccccc7c56)cc4)cc3c12 ; c1ccc2c(c1)ccc1ccc3ccc(-c4ccc(N(c5ccc6c(c5)sc5ccccc56)c5ccc6oc7ccccc7c6c5)cc4)cc3c12 ; c1ccc2c(c1)ccc1ccc3ccc(-c4ccc(N(c5ccc6c(c5)sc5ccccc56)c5ccc6sc7ccccc7c6c5)cc4)cc3c12]

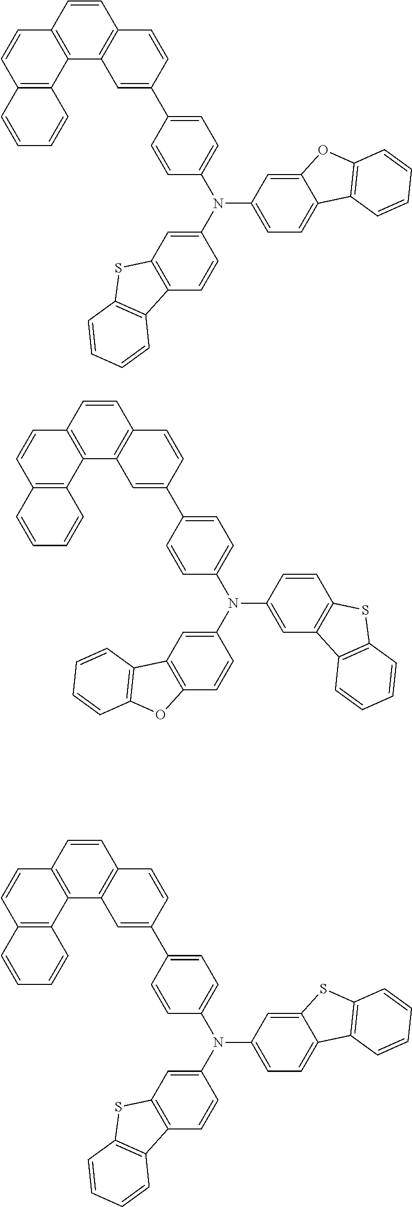

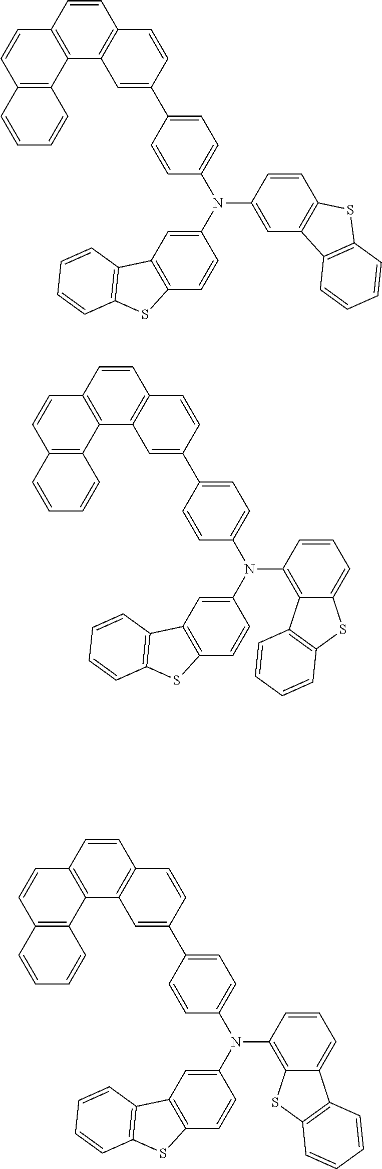 [SMILES: c1ccc2c(c1)ccc1ccc3ccc(-c4ccc(N(c5ccc6sc7ccccc7c6c5)c5ccc6sc7ccccc7c6c5)cc4)cc3c12 ; c1ccc2c(c1)ccc1ccc3ccc(-c4ccc(N(c5ccc6sc7ccccc7c6c5)c5cccc6sc7ccccc7c56)cc4)cc3c12 ; c1ccc2c(c1)ccc1ccc3ccc(-c4ccc(N(c5ccc6sc7ccccc7c6c5)c5cccc6c5sc5ccccc56)cc4)cc3c12]

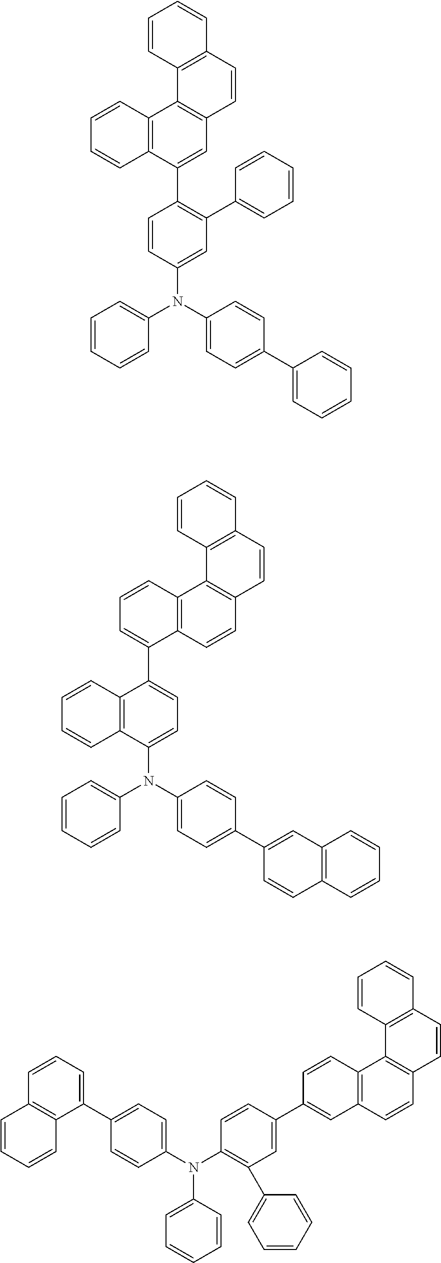 [SMILES: c1ccc(-c2ccc(N(c3ccccc3)c3ccc(-c4cc5ccc6ccccc6c5c5ccccc45)c(-c4ccccc4)c3)cc2)cc1 ; c1ccc(N(c2ccc(-c3ccc4ccccc4c3)cc2)c2ccc(-c3cccc4c3ccc3ccc5ccccc5c34)c3ccccc23)cc1 ; c1ccc(-c2cc(-c3ccc4c(ccc5ccc6ccccc6c54)c3)ccc2N(c2ccccc2)c2ccc(-c3cccc4ccccc34)cc2)cc1]

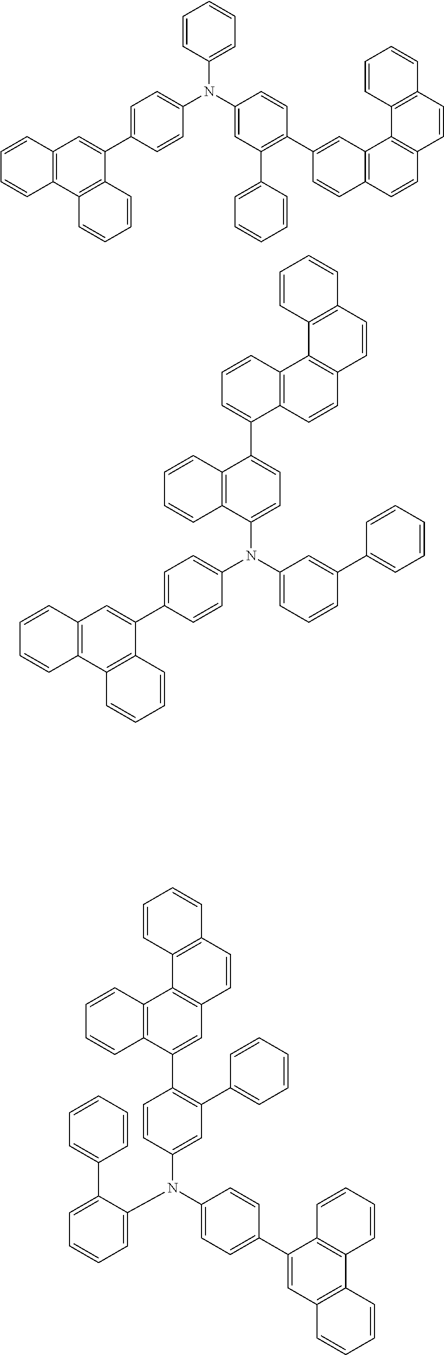

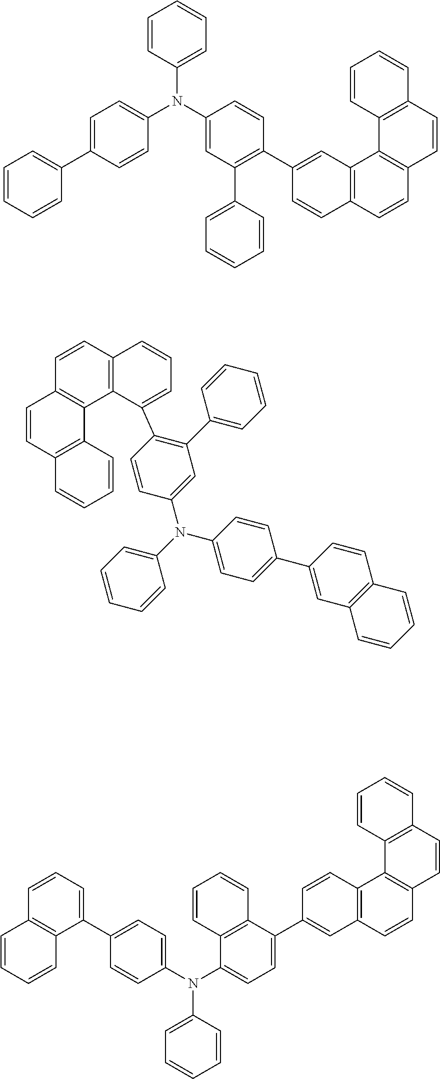 [SMILES: c1ccc(-c2ccc(N(c3ccccc3)c3ccc(-c4ccc5ccc6ccc7ccccc7c6c5c4)c(-c4ccccc4)c3)cc2)cc1 ; c1ccc(-c2cc(N(c3ccccc3)c3ccc(-c4ccc5ccccc5c4)cc3)ccc2-c2cccc3ccc4ccc5ccccc5c4c23)cc1 ; c1ccc(N(c2ccc(-c3cccc4ccccc34)cc2)c2ccc(-c3ccc4c(ccc5ccc6ccccc6c54)c3)c3ccccc23)cc1]

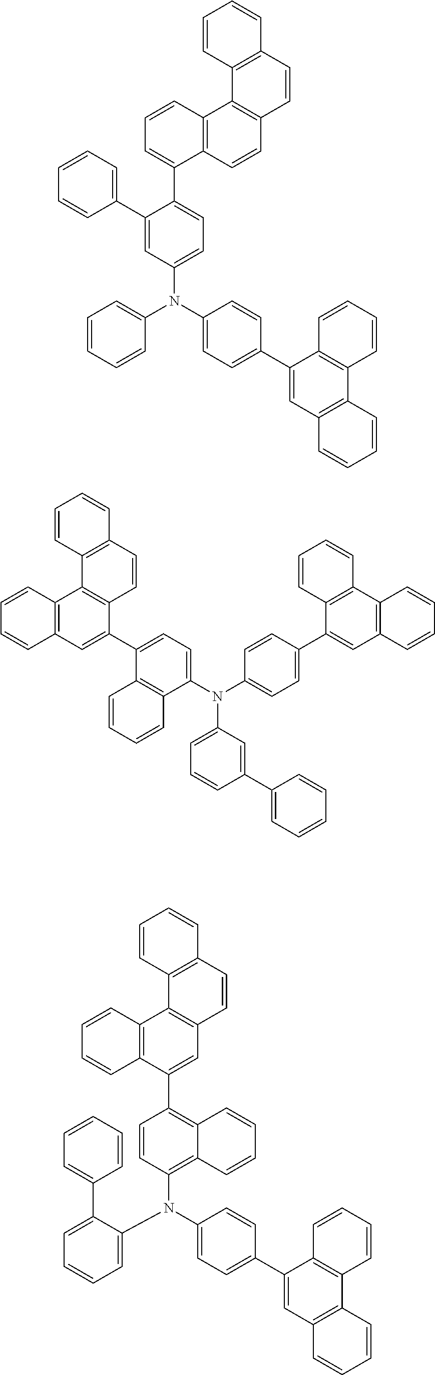 [SMILES: c1ccc(-c2cc(N(c3ccccc3)c3ccc(-c4cc5ccccc5c5ccccc45)cc3)ccc2-c2cccc3c2ccc2ccc4ccccc4c23)cc1 ; c1ccc(-c2cccc(N(c3ccc(-c4cc5ccccc5c5ccccc45)cc3)c3ccc(-c4cc5ccccc5c5c4ccc4ccccc45)c4ccccc34)c2)cc1 ; c1ccc(-c2ccccc2N(c2ccc(-c3cc4ccccc4c4ccccc34)cc2)c2ccc(-c3cc4ccc5ccccc5c4c4ccccc34)c3ccccc23)cc1]

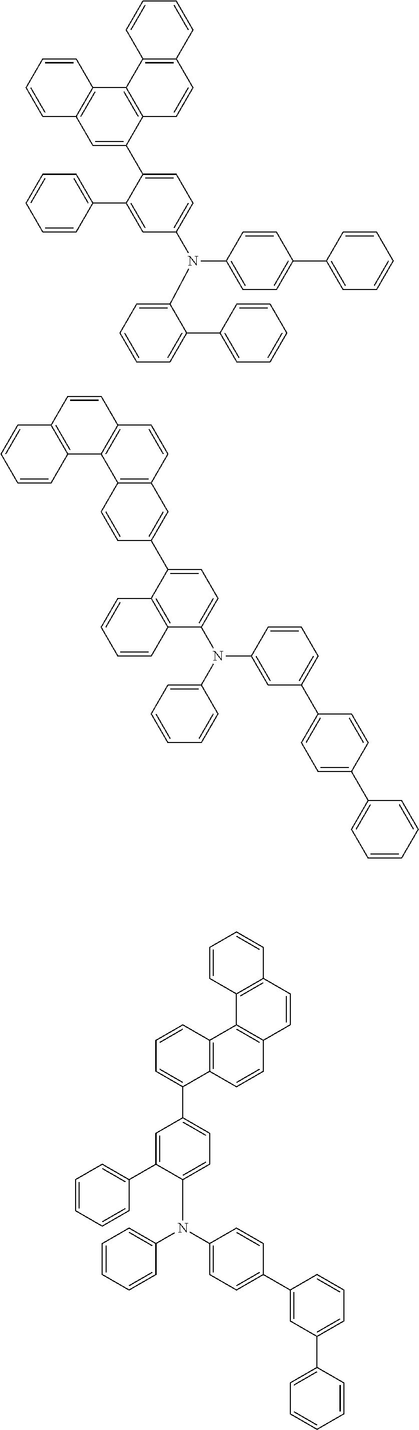 [SMILES: c1ccc(-c2ccc(N(c3ccc(-c4cc5ccccc5c5c4ccc4ccccc45)c(-c4ccccc4)c3)c3ccccc3-c3ccccc3)cc2)cc1 ; c1ccc(-c2ccc(-c3cccc(N(c4ccccc4)c4ccc(-c5ccc6c(ccc7ccc8ccccc8c76)c5)c5ccccc45)c3)cc2)cc1 ; c1ccc(-c2cccc(-c3ccc(N(c4ccccc4)c4ccc(-c5cccc6c5ccc5ccc7ccccc7c56)cc4-c4ccccc4)cc3)c2)cc1]

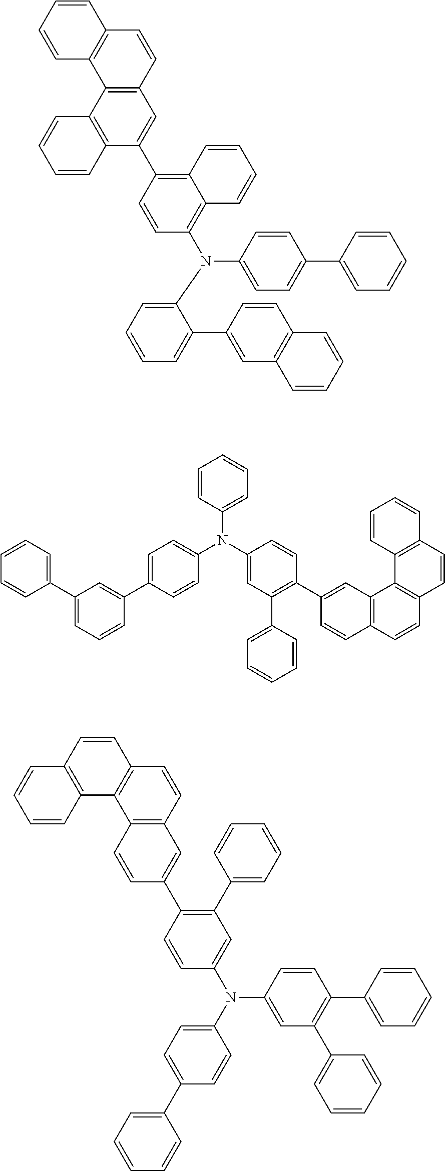 [SMILES: c1ccc(-c2ccc(N(c3ccccc3-c3ccc4ccccc4c3)c3ccc(-c4cc5ccc6ccccc6c5c5ccccc45)c4ccccc34)cc2)cc1 ; c1ccc(-c2cccc(-c3ccc(N(c4ccccc4)c4ccc(-c5ccc6ccc7ccc8ccccc8c7c6c5)c(-c5ccccc5)c4)cc3)c2)cc1 ; c1ccc(-c2ccc(N(c3ccc(-c4ccccc4)c(-c4ccccc4)c3)c3ccc(-c4ccc5c(ccc6ccc7ccccc7c65)c4)c(-c4ccccc4)c3)cc2)cc1]

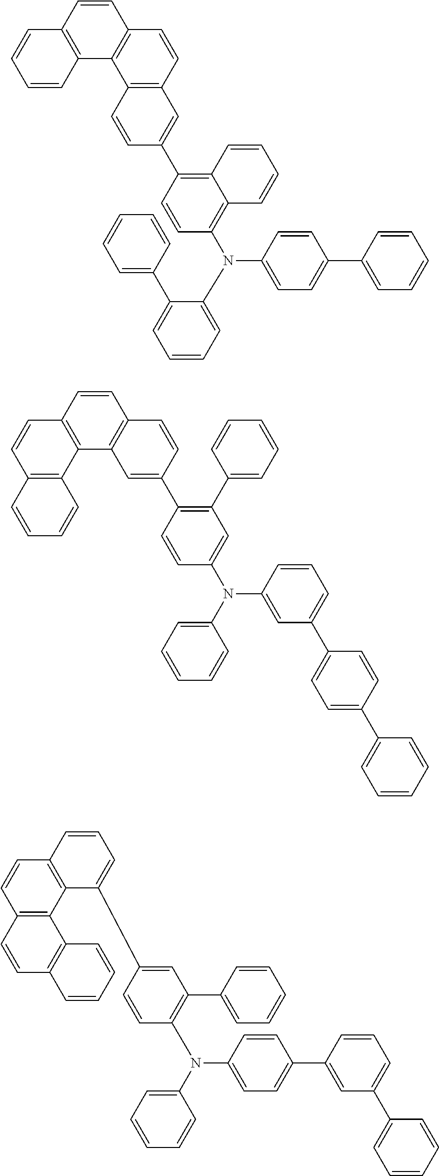 [SMILES: c1ccc(-c2ccc(N(c3ccccc3-c3ccccc3)c3ccc(-c4ccc5c(ccc6ccc7ccccc7c65)c4)c4ccccc34)cc2)cc1 ; c1ccc(-c2ccc(-c3cccc(N(c4ccccc4)c4ccc(-c5ccc6ccc7ccc8ccccc8c7c6c5)c(-c5ccccc5)c4)c3)cc2)cc1 ; c1ccc(-c2cccc(-c3ccc(N(c4ccccc4)c4ccc(-c5cccc6ccc7ccc8ccccc8c7c56)cc4-c4ccccc4)cc3)c2)cc1]

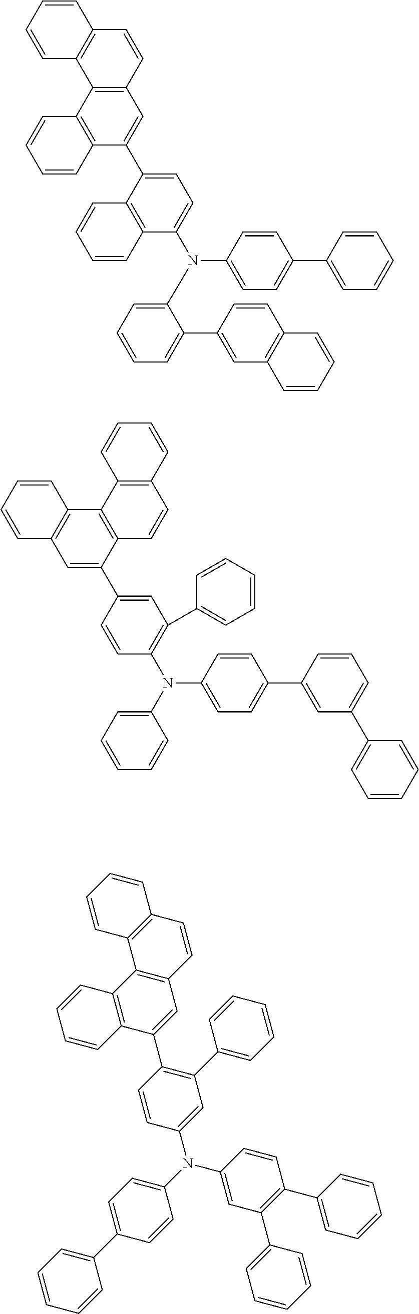 [SMILES: c1ccc(-c2ccc(N(c3ccccc3-c3ccc4ccccc4c3)c3ccc(-c4cc5ccc6ccccc6c5c5ccccc45)c4ccccc34)cc2)cc1 ; c1ccc(-c2cccc(-c3ccc(N(c4ccccc4)c4ccc(-c5cc6ccccc6c6c5ccc5ccccc56)cc4-c4ccccc4)cc3)c2)cc1 ; c1ccc(-c2ccc(N(c3ccc(-c4ccccc4)c(-c4ccccc4)c3)c3ccc(-c4cc5ccc6ccccc6c5c5ccccc45)c(-c4ccccc4)c3)cc2)cc1]

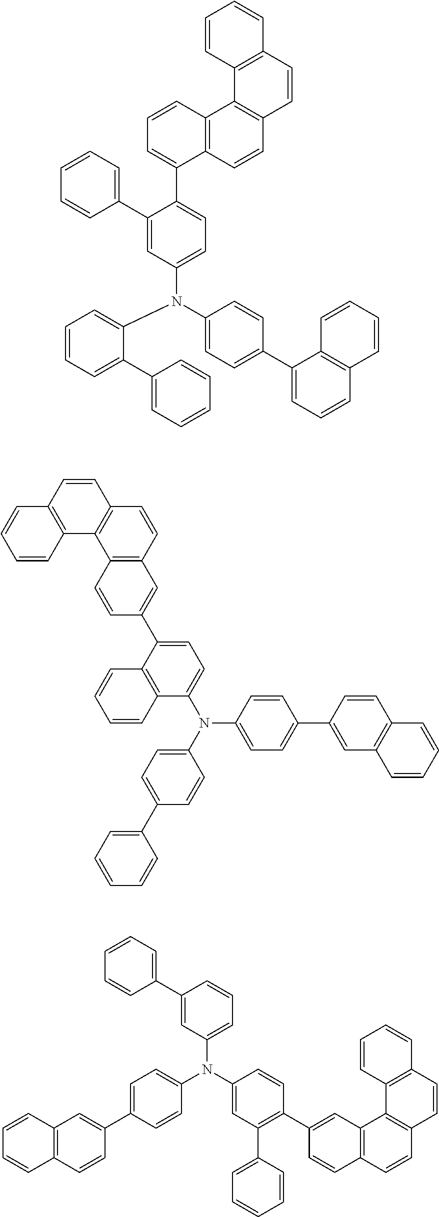 [SMILES: c1ccc(-c2cc(N(c3ccc(-c4cccc5ccccc45)cc3)c3ccccc3-c3ccccc3)ccc2-c2cccc3c2ccc2ccc4ccccc4c23)cc1 ; c1ccc(-c2ccc(N(c3ccc(-c4ccc5ccccc5c4)cc3)c3ccc(-c4ccc5c(ccc6ccc7ccccc7c65)c4)c4ccccc34)cc2)cc1 ; c1ccc(-c2cccc(N(c3ccc(-c4ccc5ccccc5c4)cc3)c3ccc(-c4ccc5ccc6ccc7ccccc7c6c5c4)c(-c4ccccc4)c3)c2)cc1]

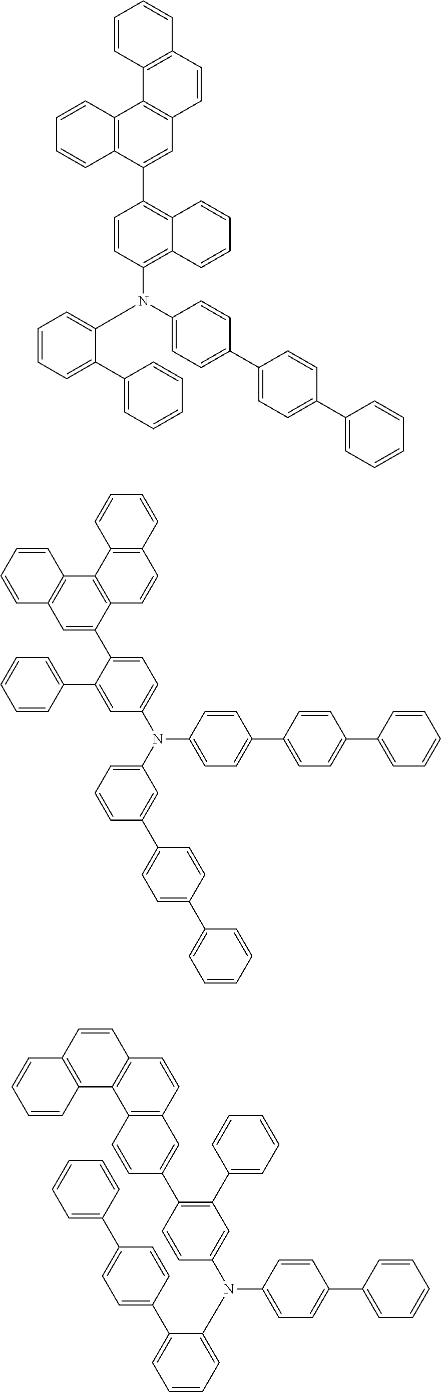 [SMILES: c1ccc(-c2ccc(-c3ccc(N(c4ccccc4-c4ccccc4)c4ccc(-c5cc6ccc7ccccc7c6c6ccccc56)c5ccccc45)cc3)cc2)cc1 ; c1ccc(-c2ccc(-c3ccc(N(c4cccc(-c5ccc(-c6ccccc6)cc5)c4)c4ccc(-c5cc6ccccc6c6c5ccc5ccccc56)c(-c5ccccc5)c4)cc3)cc2)cc1 ; c1ccc(-c2ccc(-c3ccccc3N(c3ccc(-c4ccccc4)cc3)c3ccc(-c4ccc5c(ccc6ccc7ccccc7c65)c4)c(-c4ccccc4)c3)cc2)cc1]

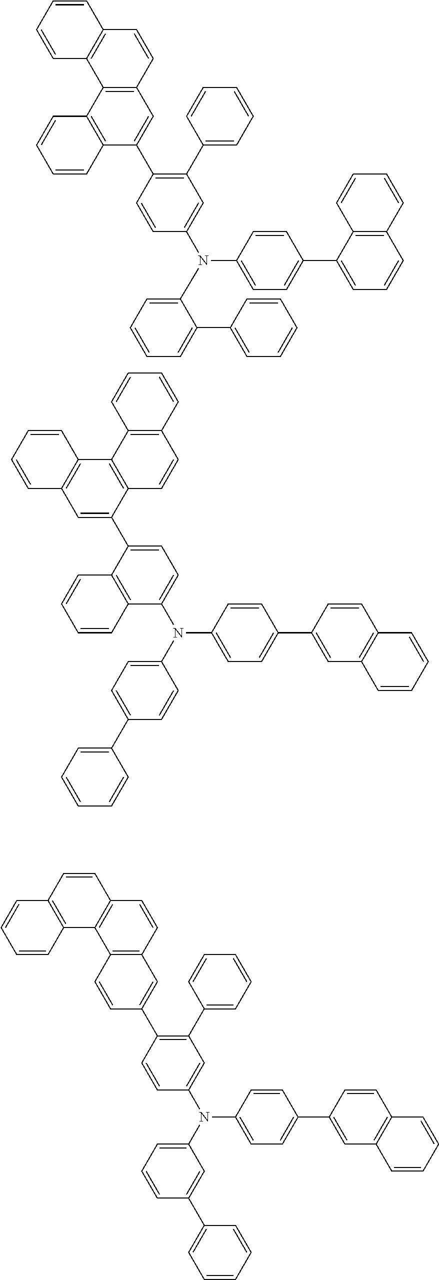

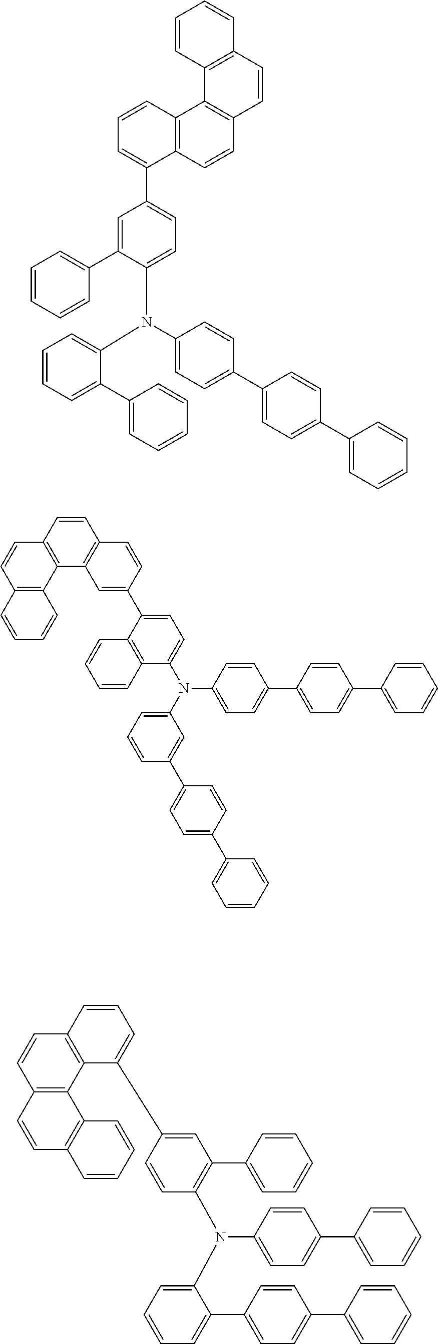 [SMILES: c1ccc(-c2ccc(-c3ccc(N(c4ccccc4-c4ccccc4)c4ccc(-c5cccc6c5ccc5ccc7ccccc7c56)cc4-c4ccccc4)cc3)cc2)cc1 ; c1ccc(-c2ccc(-c3ccc(N(c4cccc(-c5ccc(-c6ccccc6)cc5)c4)c4ccc(-c5ccc6ccc7ccc8ccccc8c7c6c5)c5ccccc45)cc3)cc2)cc1 ; c1ccc(-c2ccc(-c3ccccc3N(c3ccc(-c4ccccc4)cc3)c3ccc(-c4cccc5ccc6ccc7ccccc7c6c45)cc3-c3ccccc3)cc2)cc1]

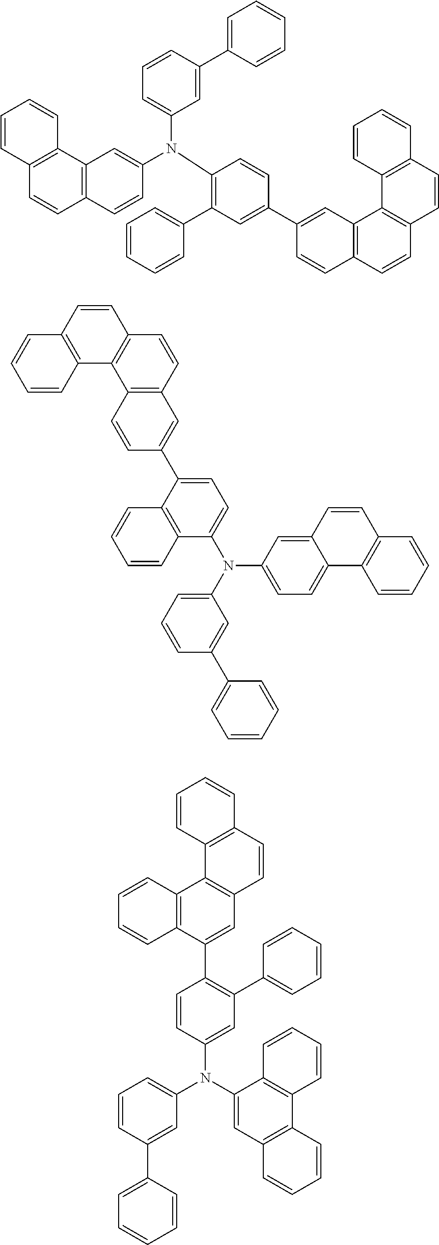

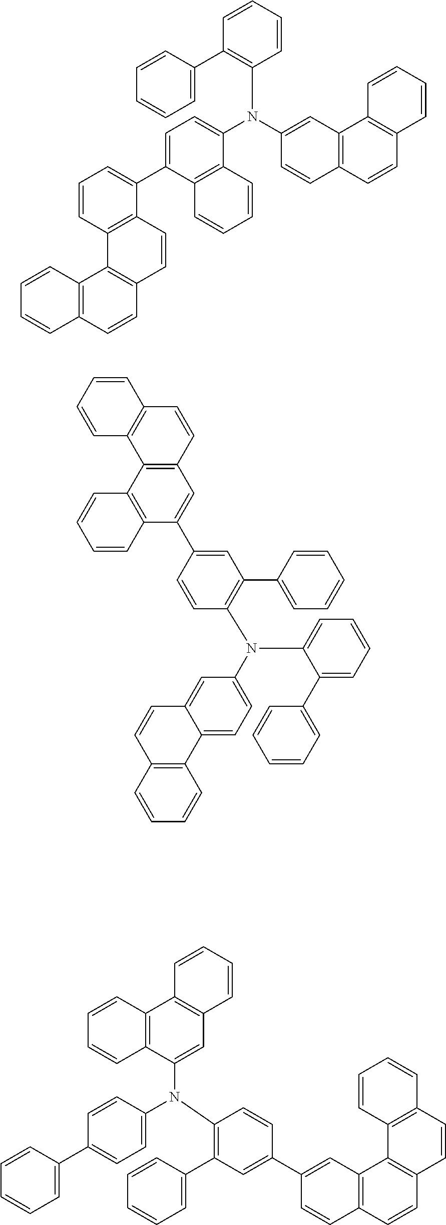

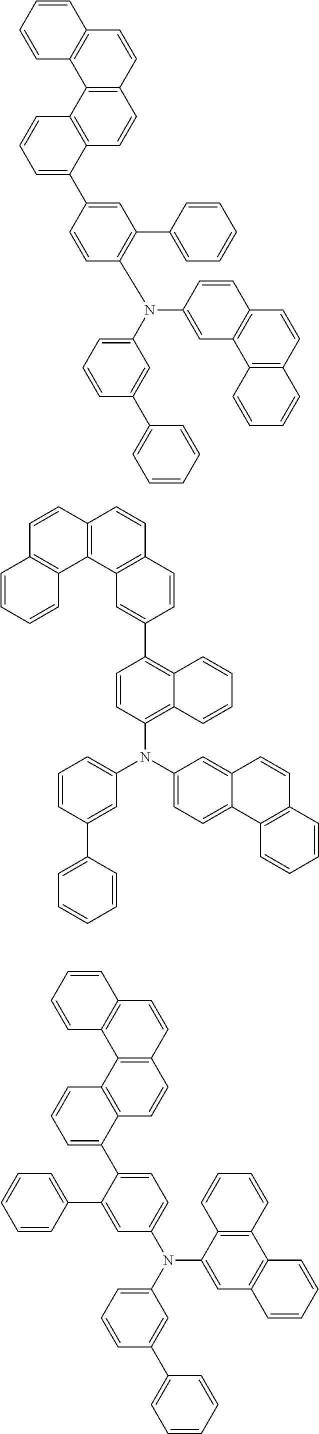

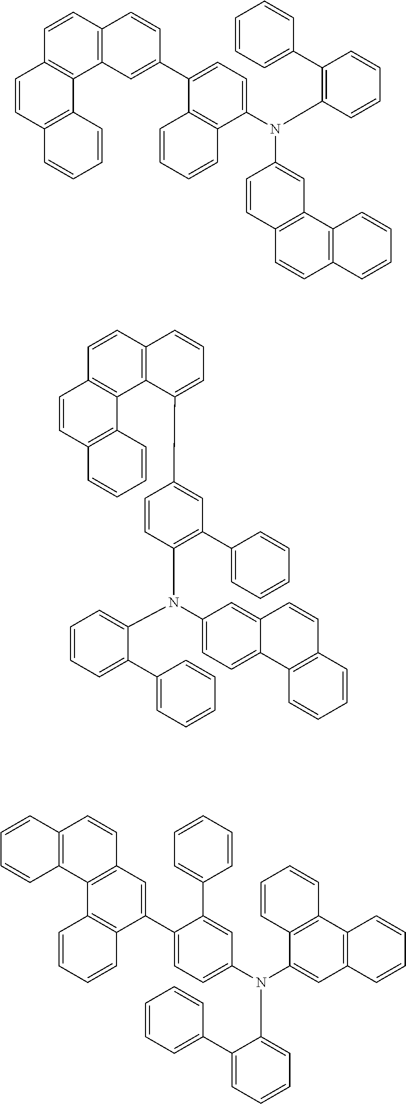 [SMILES: c1ccc(-c2ccccc2N(c2ccc3ccc4ccccc4c3c2)c2ccc(-c3ccc4ccc5ccc6ccccc6c5c4c3)c3ccccc23)cc1 ; c1ccc(-c2ccccc2N(c2ccc3c(ccc4ccccc43)c2)c2ccc(-c3cccc4ccc5ccc6ccccc6c5c34)cc2-c2ccccc2)cc1 ; c1ccc(-c2cc(N(c3ccccc3-c3ccccc3)c3cc4ccccc4c4ccccc34)ccc2-c2cc3ccc4ccccc4c3c3ccccc23)cc1]

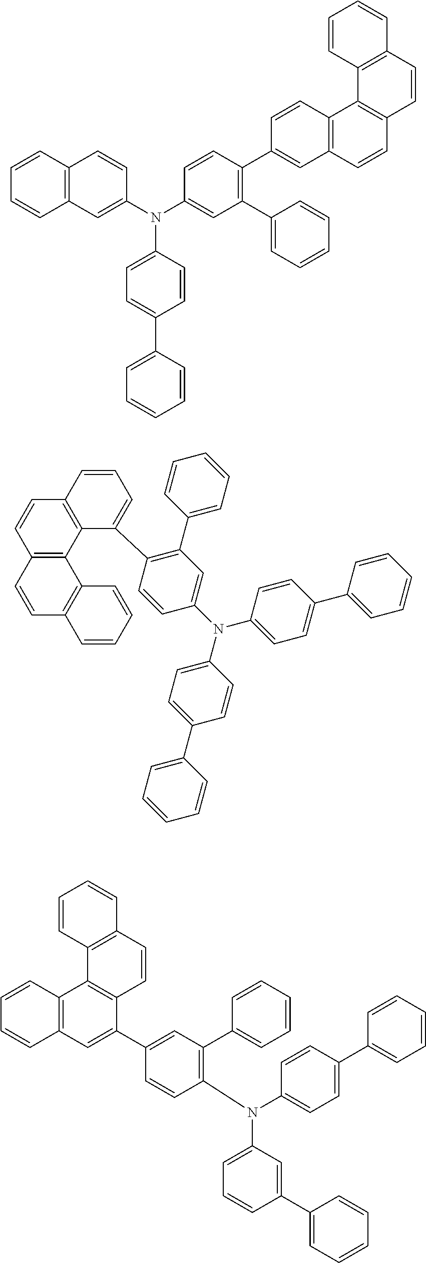 [SMILES: c1ccc(-c2ccc(N(c3ccc(-c4ccc5c(ccc6ccc7ccccc7c65)c4)c(-c4ccccc4)c3)c3ccc4ccccc4c3)cc2)cc1 ; c1ccc(-c2ccc(N(c3ccc(-c4ccccc4)cc3)c3ccc(-c4cccc5ccc6ccc7ccccc7c6c45)c(-c4ccccc4)c3)cc2)cc1 ; c1ccc(-c2ccc(N(c3cccc(-c4ccccc4)c3)c3ccc(-c4cc5ccccc5c5c4ccc4ccccc45)cc3-c3ccccc3)cc2)cc1]

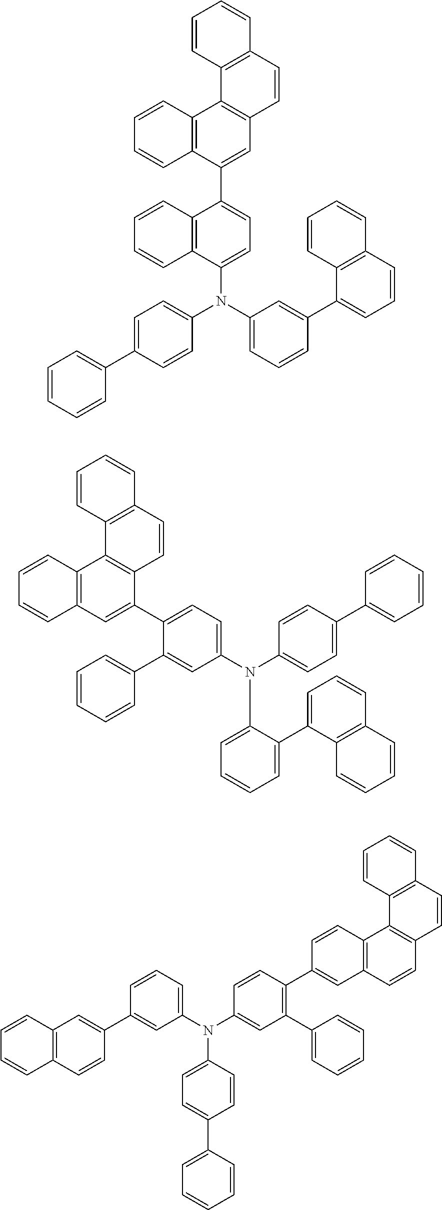 [SMILES: c1ccc(-c2ccc(N(c3cccc(-c4cccc5ccccc45)c3)c3ccc(-c4cc5ccc6ccccc6c5c5ccccc45)c4ccccc34)cc2)cc1 ; c1ccc(-c2ccc(N(c3ccc(-c4cc5ccccc5c5c4ccc4ccccc45)c(-c4ccccc4)c3)c3ccccc3-c3cccc4ccccc34)cc2)cc1 ; c1ccc(-c2ccc(N(c3cccc(-c4ccc5ccccc5c4)c3)c3ccc(-c4ccc5c(ccc6ccc7ccccc7c65)c4)c(-c4ccccc4)c3)cc2)cc1]

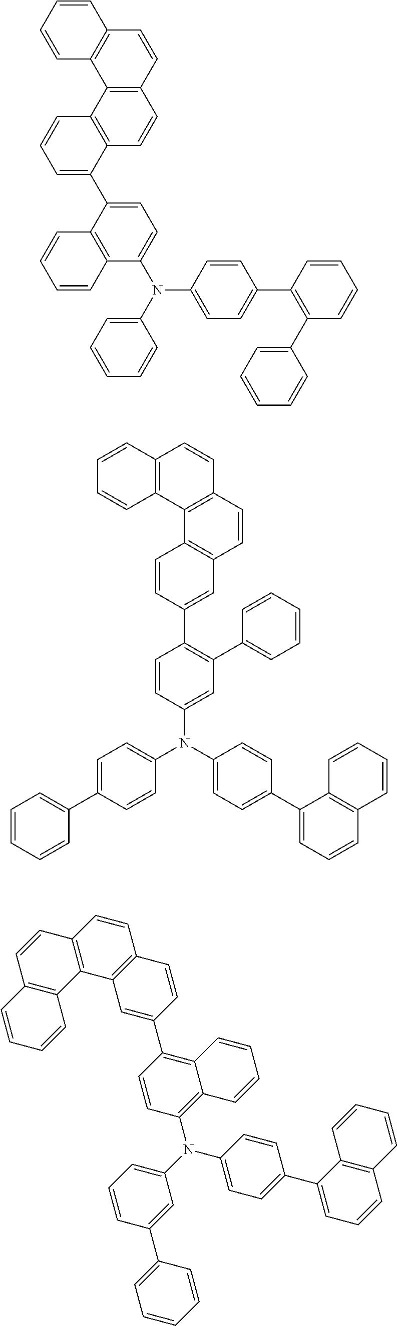 [SMILES: c1ccc(-c2ccccc2-c2ccc(N(c3ccccc3)c3ccc(-c4cccc5c4ccc4ccc6ccccc6c45)c4ccccc34)cc2)cc1 ; c1ccc(-c2ccc(N(c3ccc(-c4cccc5ccccc45)cc3)c3ccc(-c4ccc5c(ccc6ccc7ccccc7c65)c4)c(-c4ccccc4)c3)cc2)cc1 ; c1ccc(-c2cccc(N(c3ccc(-c4cccc5ccccc45)cc3)c3ccc(-c4ccc5ccc6ccc7ccccc7c6c5c4)c4ccccc34)c2)cc1]

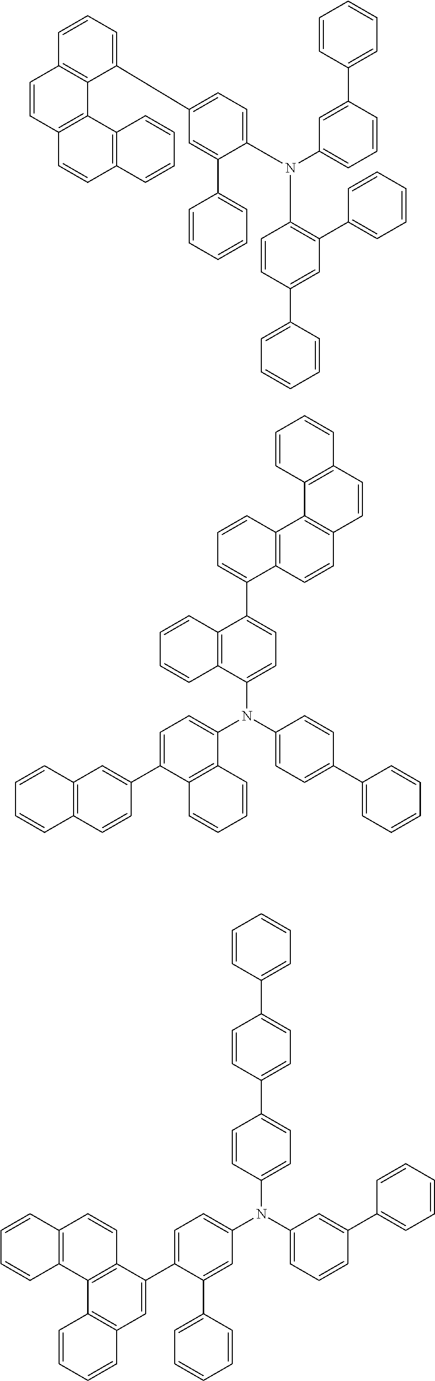

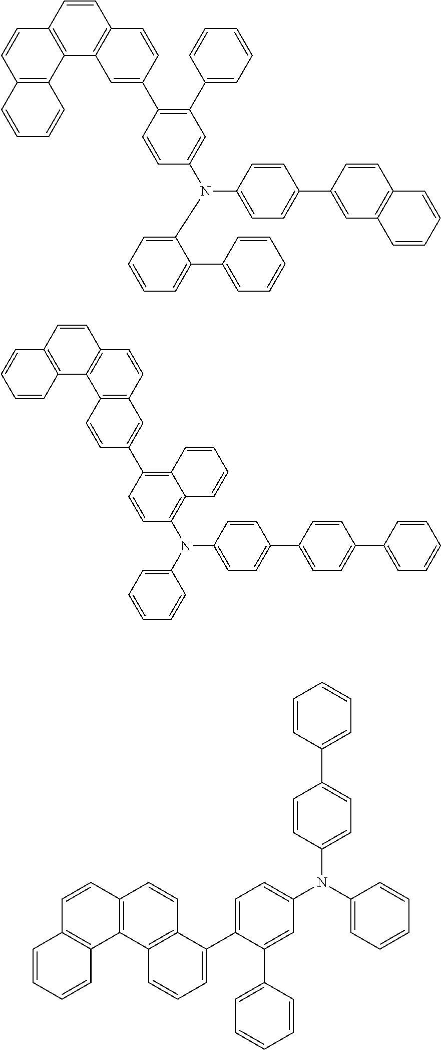 [SMILES: c1ccc(-c2cc(N(c3ccc(-c4ccc5ccccc5c4)cc3)c3ccccc3-c3ccccc3)ccc2-c2ccc3ccc4ccc5ccccc5c4c3c2)cc1 ; c1ccc(-c2ccc(-c3ccc(N(c4ccccc4)c4ccc(-c5ccc6c(ccc7ccc8ccccc8c76)c5)c5ccccc45)cc3)cc2)cc1 ; c1ccc(-c2ccc(N(c3ccccc3)c3ccc(-c4cccc5c4ccc4ccc6ccccc6c45)c(-c4ccccc4)c3)cc2)cc1]

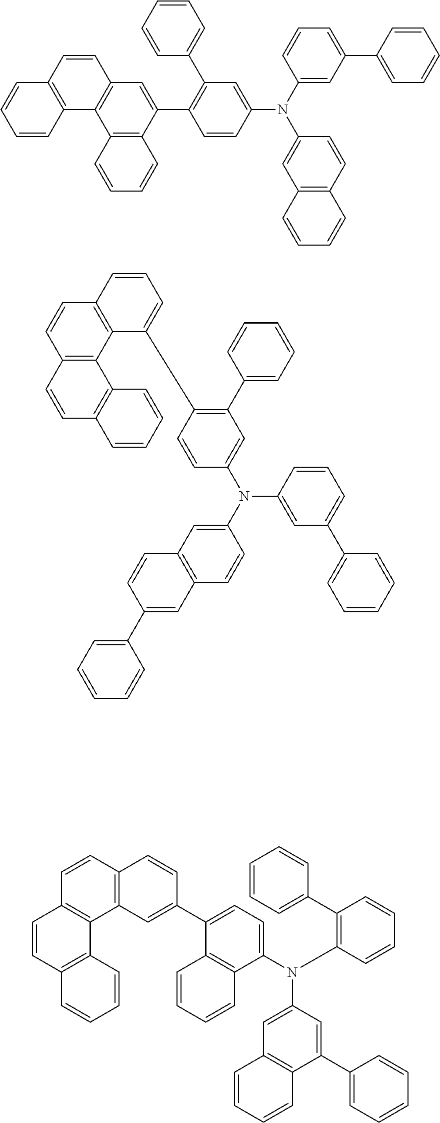 [SMILES: c1ccc(-c2cccc(N(c3ccc(-c4cc5ccc6ccccc6c5c5ccccc45)c(-c4ccccc4)c3)c3ccc4ccccc4c3)c2)cc1 ; c1ccc(-c2cccc(N(c3ccc(-c4cccc5ccc6ccc7ccccc7c6c45)c(-c4ccccc4)c3)c3ccc4cc(-c5ccccc5)ccc4c3)c2)cc1 ; c1ccc(-c2ccccc2N(c2cc(-c3ccccc3)c3ccccc3c2)c2ccc(-c3ccc4ccc5ccc6ccccc6c5c4c3)c3ccccc23)cc1]

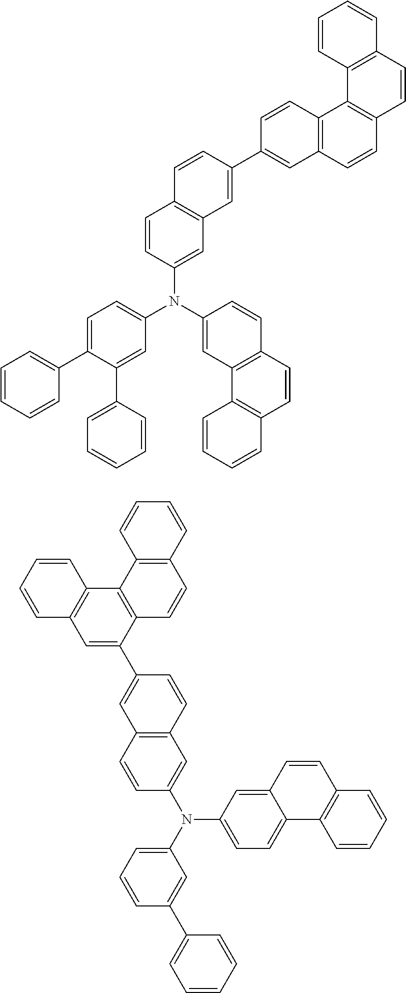

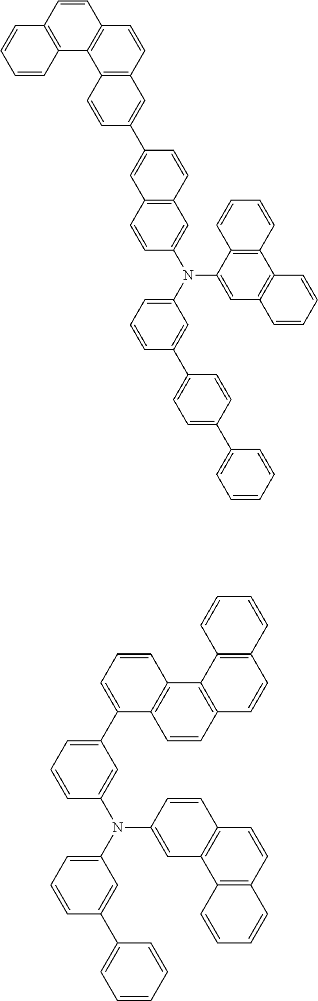 [SMILES: c1ccc(-c2ccc(-c3cccc(N(c4ccc5cc(-c6ccc7c(ccc8ccc9ccccc9c87)c6)ccc5c4)c4cc5ccccc5c5ccccc45)c3)cc2)cc1 ; c1ccc(-c2cccc(N(c3cccc(-c4cccc5c4ccc4ccc6ccccc6c45)c3)c3ccc4ccc5ccccc5c4c3)c2)cc1]

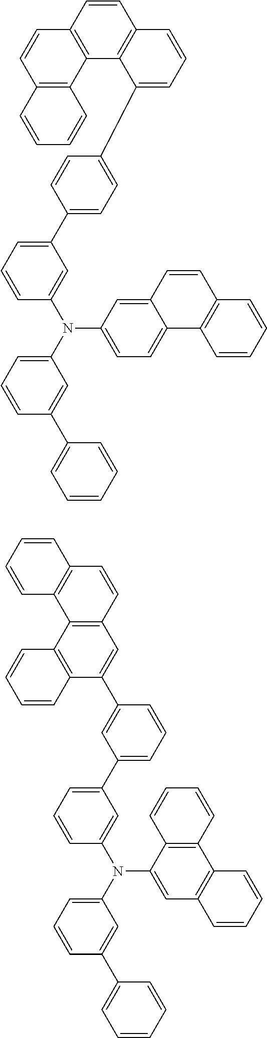

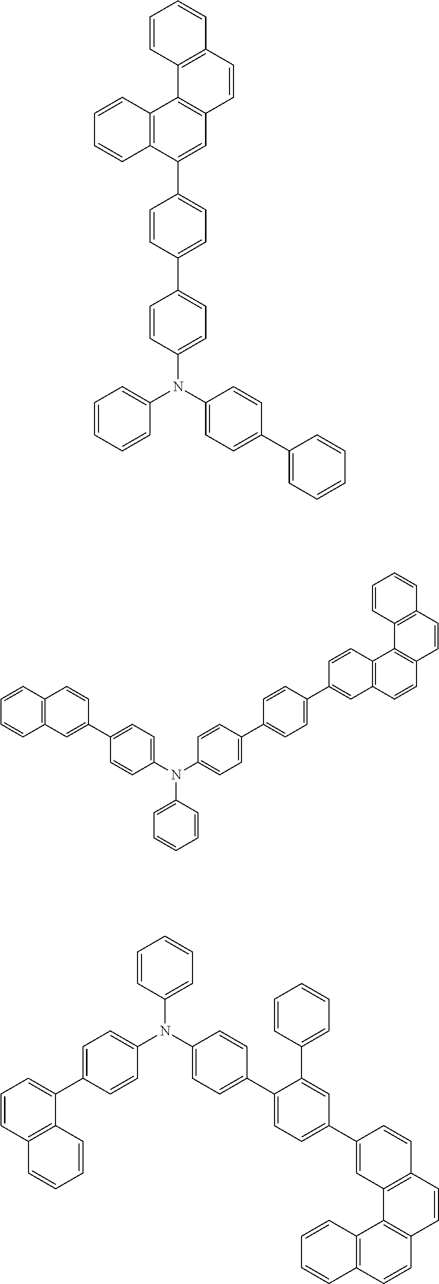 [SMILES: c1ccc(-c2ccc(N(c3ccccc3)c3ccc(-c4ccc(-c5cc6ccc7ccccc7c6c6ccccc56)cc4)cc3)cc2)cc1 ; c1ccc(N(c2ccc(-c3ccc(-c4ccc5c(ccc6ccc7ccccc7c65)c4)cc3)cc2)c2ccc(-c3ccc4ccccc4c3)cc2)cc1 ; c1ccc(-c2cc(-c3ccc4ccc5ccc6ccccc6c5c4c3)ccc2-c2ccc(N(c3ccccc3)c3ccc(-c4cccc5ccccc45)cc3)cc2)cc1]

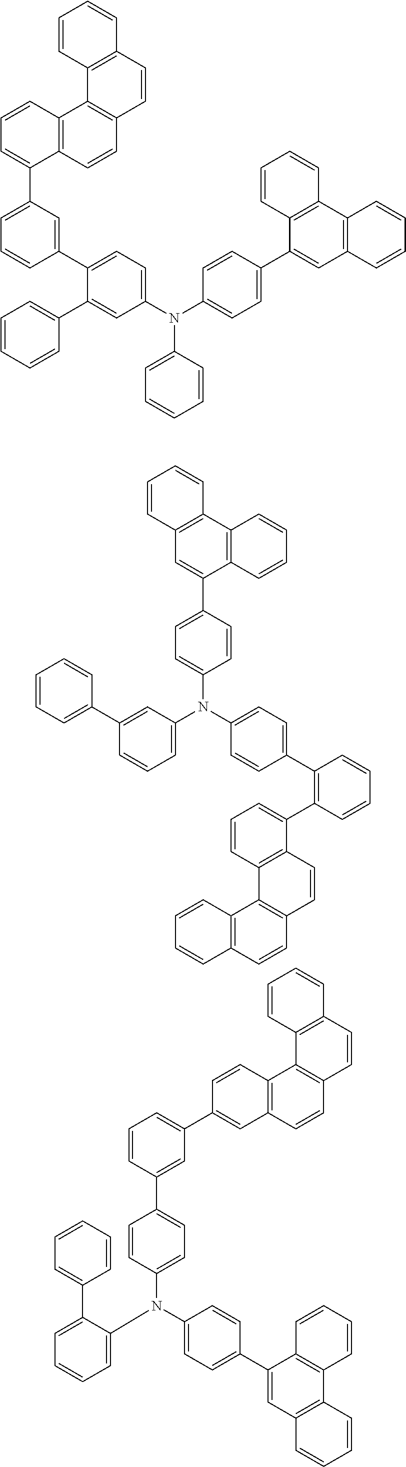

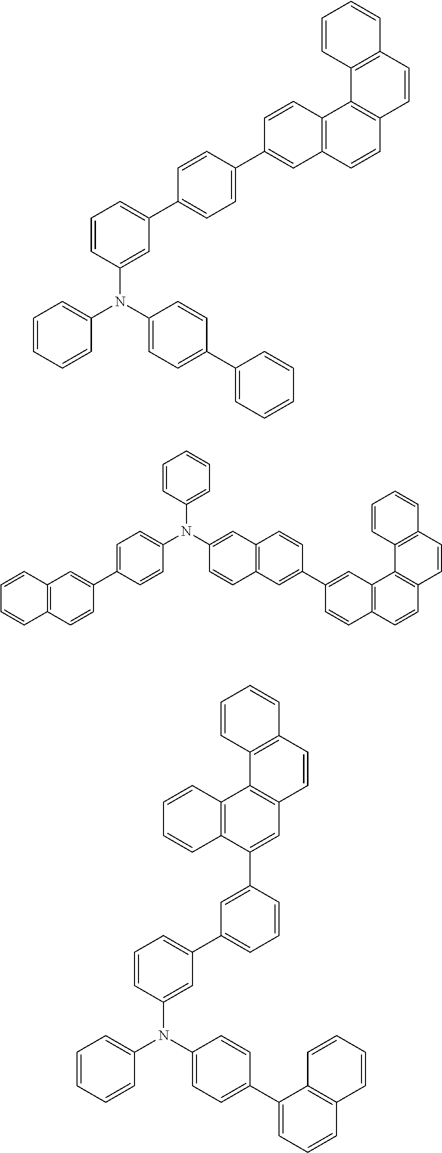 [SMILES: c1ccc(-c2ccc(N(c3ccccc3)c3cccc(-c4ccc(-c5ccc6c(ccc7ccc8ccccc8c76)c5)cc4)c3)cc2)cc1 ; c1ccc(N(c2ccc(-c3ccc4ccccc4c3)cc2)c2ccc3cc(-c4ccc5ccc6ccc7ccccc7c6c5c4)ccc3c2)cc1 ; c1ccc(N(c2ccc(-c3cccc4ccccc34)cc2)c2cccc(-c3cccc(-c4cc5ccc6ccccc6c5c5ccccc45)c3)c2)cc1]

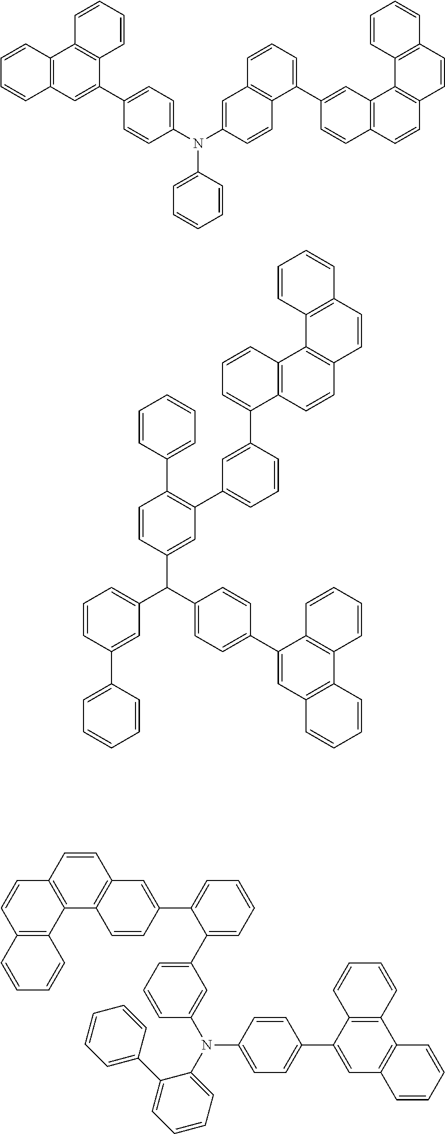

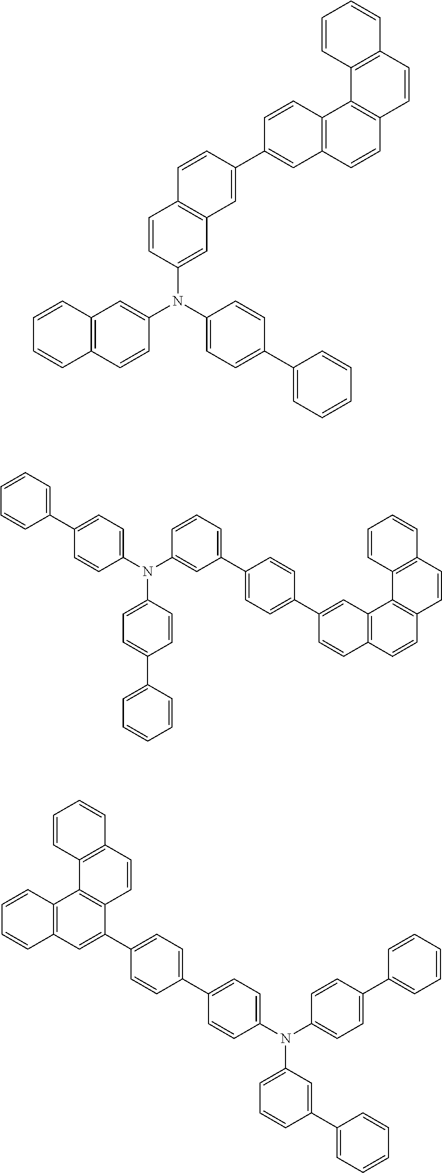 [SMILES: c1ccc(-c2ccc(N(c3ccc4ccccc4c3)c3ccc4ccc(-c5ccc6c(ccc7ccc8ccccc8c76)c5)cc4c3)cc2)cc1 ; c1ccc(-c2ccc(N(c3ccc(-c4ccccc4)cc3)c3cccc(-c4ccc(-c5ccc6ccc7ccc8ccccc8c7c6c5)cc4)c3)cc2)cc1 ; c1ccc(-c2ccc(N(c3ccc(-c4ccc(-c5cc6ccccc6c6c5ccc5ccccc56)cc4)cc3)c3cccc(-c4ccccc4)c3)cc2)cc1]

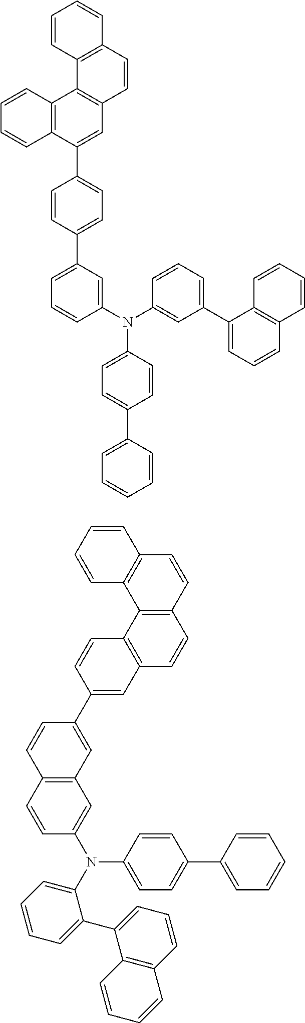

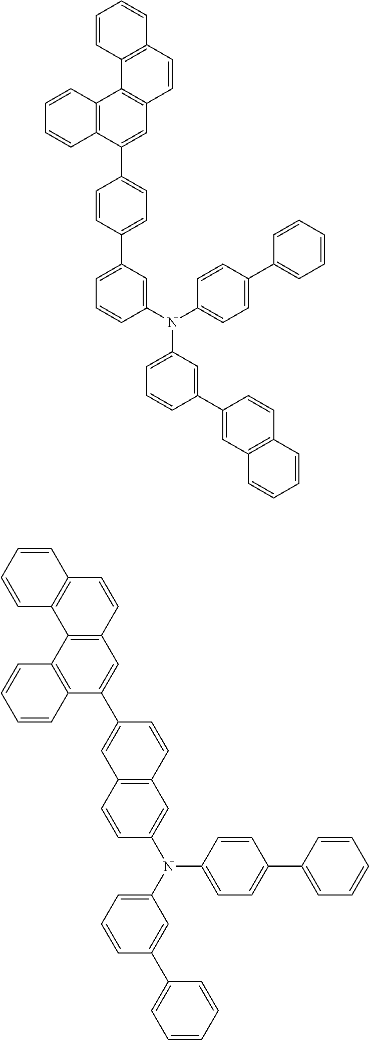 [SMILES: c1ccc(-c2ccc(N(c3cccc(-c4ccc(-c5cc6ccc7ccccc7c6c6ccccc56)cc4)c3)c3cccc(-c4ccc5ccccc5c4)c3)cc2)cc1 ; c1ccc(-c2ccc(N(c3cccc(-c4ccccc4)c3)c3ccc4cc(-c5cc6ccc7ccccc7c6c6ccccc56)ccc4c3)cc2)cc1]

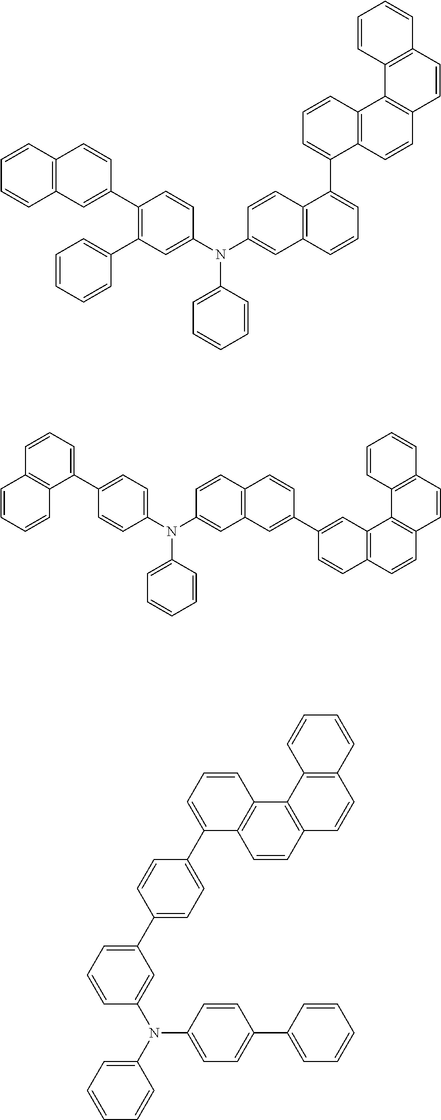 [SMILES: c1ccc(-c2cc(N(c3ccccc3)c3ccc4c(-c5cccc6c5ccc5ccc7ccccc7c56)cccc4c3)ccc2-c2ccc3ccccc3c2)cc1 ; c1ccc(N(c2ccc(-c3cccc4ccccc34)cc2)c2ccc3ccc(-c4ccc5ccc6ccc7ccccc7c6c5c4)cc3c2)cc1 ; c1ccc(-c2ccc(N(c3ccccc3)c3cccc(-c4ccc(-c5cccc6c5ccc5ccc7ccccc7c56)cc4)c3)cc2)cc1]

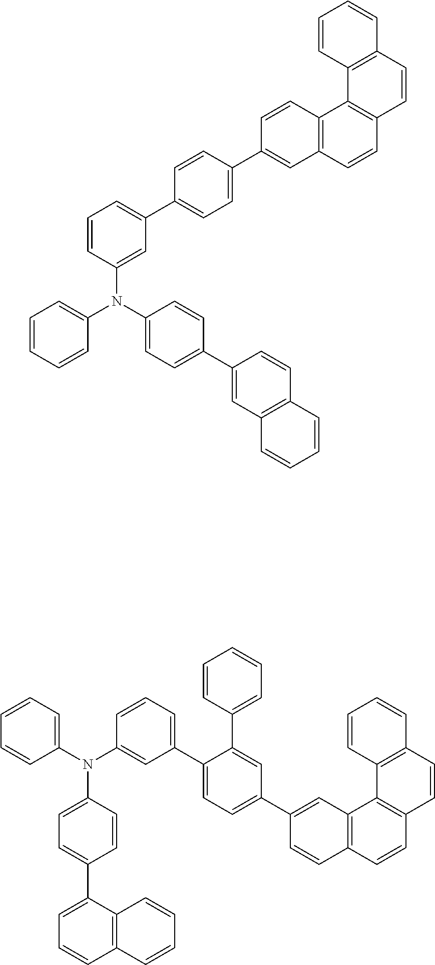 [SMILES: c1ccc(N(c2ccc(-c3ccc4ccccc4c3)cc2)c2cccc(-c3ccc(-c4ccc5c(ccc6ccc7ccccc7c65)c4)cc3)c2)cc1 ; c1ccc(-c2cc(-c3ccc4ccc5ccc6ccccc6c5c4c3)ccc2-c2cccc(N(c3ccccc3)c3ccc(-c4cccc5ccccc45)cc3)c2)cc1]

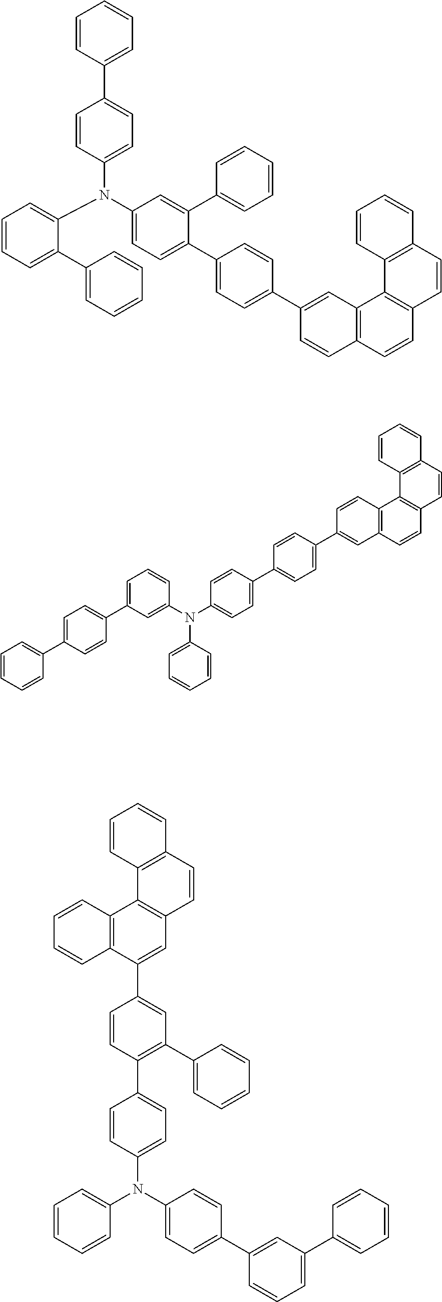 [SMILES: c1ccc(-c2ccc(N(c3ccc(-c4ccc(-c5ccc6ccc7ccc8ccccc8c7c6c5)cc4)c(-c4ccccc4)c3)c3ccccc3-c3ccccc3)cc2)cc1 ; c1ccc(-c2ccc(-c3cccc(N(c4ccccc4)c4ccc(-c5ccc(-c6ccc7c(ccc8ccc9ccccc9c87)c6)cc5)cc4)c3)cc2)cc1 ; c1ccc(-c2cccc(-c3ccc(N(c4ccccc4)c4ccc(-c5ccc(-c6cc7ccc8ccccc8c7c7ccccc67)cc5-c5ccccc5)cc4)cc3)c2)cc1]

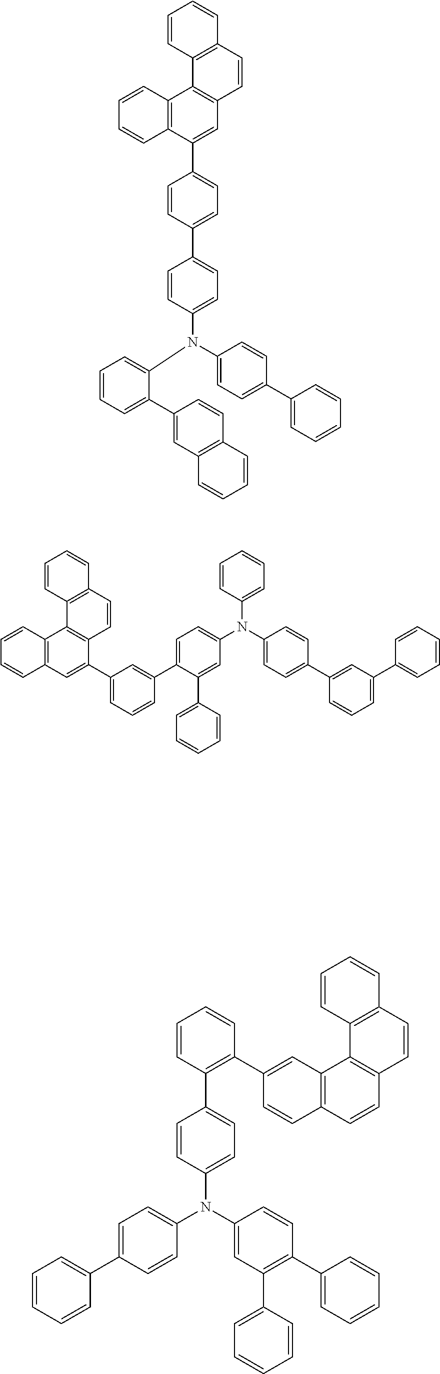 [SMILES: c1ccc(-c2ccc(N(c3ccc(-c4ccc(-c5cc6ccc7ccccc7c6c6ccccc56)cc4)cc3)c3ccccc3-c3ccc4ccccc4c3)cc2)cc1 ; c1ccc(-c2cccc(-c3ccc(N(c4ccccc4)c4ccc(-c5cccc(-c6cc7ccccc7c7c6ccc6ccccc67)c5)c(-c5ccccc5)c4)cc3)c2)cc1 ; c1ccc(-c2ccc(N(c3ccc(-c4ccccc4-c4ccc5ccc6ccc7ccccc7c6c5c4)cc3)c3ccc(-c4ccccc4)c(-c4ccccc4)c3)cc2)cc1]

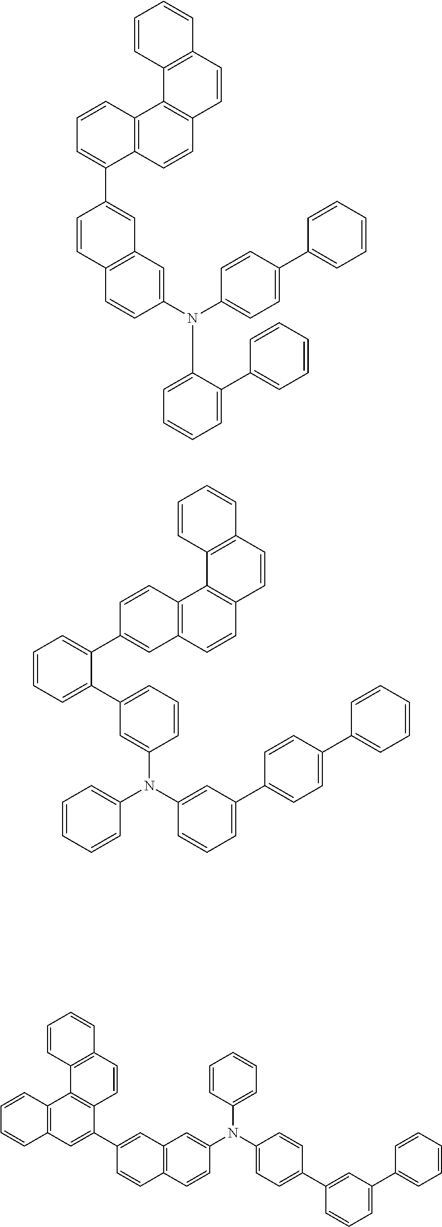 [SMILES: c1ccc(-c2ccc(N(c3ccc4ccc(-c5cccc6c5ccc5ccc7ccccc7c56)cc4c3)c3ccccc3-c3ccccc3)cc2)cc1 ; c1ccc(-c2ccc(-c3cccc(N(c4ccccc4)c4cccc(-c5ccccc5-c5ccc6c(ccc7ccc8ccccc8c76)c5)c4)c3)cc2)cc1 ; c1ccc(-c2cccc(-c3ccc(N(c4ccccc4)c4ccc5ccc(-c6cc7ccccc7c7c6ccc6ccccc67)cc5c4)cc3)c2)cc1]

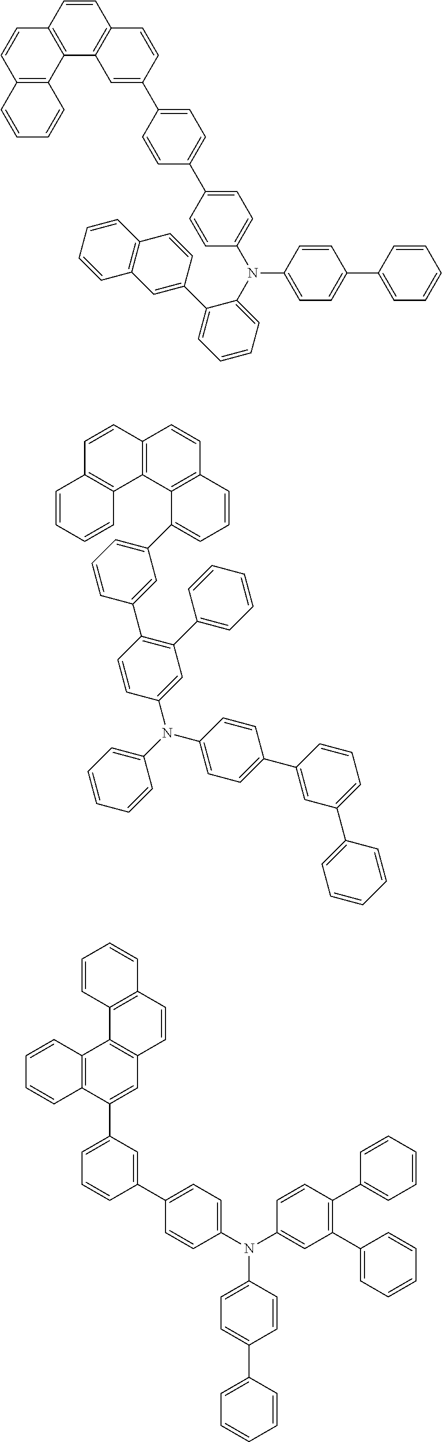 [SMILES: c1ccc(-c2ccc(N(c3ccc(-c4ccc(-c5ccc6ccc7ccc8ccccc8c7c6c5)cc4)cc3)c3ccccc3-c3ccc4ccccc4c3)cc2)cc1 ; c1ccc(-c2cccc(-c3ccc(N(c4ccccc4)c4ccc(-c5cccc(-c6cccc7ccc8ccc9ccccc9c8c67)c5)c(-c5ccccc5)c4)cc3)c2)cc1 ; c1ccc(-c2ccc(N(c3ccc(-c4cccc(-c5cc6ccc7ccccc7c6c6ccccc56)c4)cc3)c3ccc(-c4ccccc4)c(-c4ccccc4)c3)cc2)cc1]

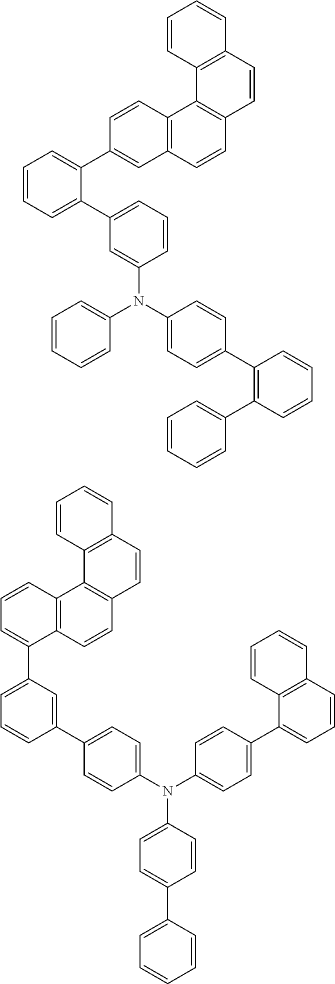 [SMILES: c1ccc(-c2ccccc2-c2ccc(N(c3ccccc3)c3cccc(-c4ccccc4-c4ccc5c(ccc6ccc7ccccc7c65)c4)c3)cc2)cc1 ; c1ccc(-c2ccc(N(c3ccc(-c4cccc(-c5cccc6c5ccc5ccc7ccccc7c56)c4)cc3)c3ccc(-c4cccc5ccccc45)cc3)cc2)cc1]

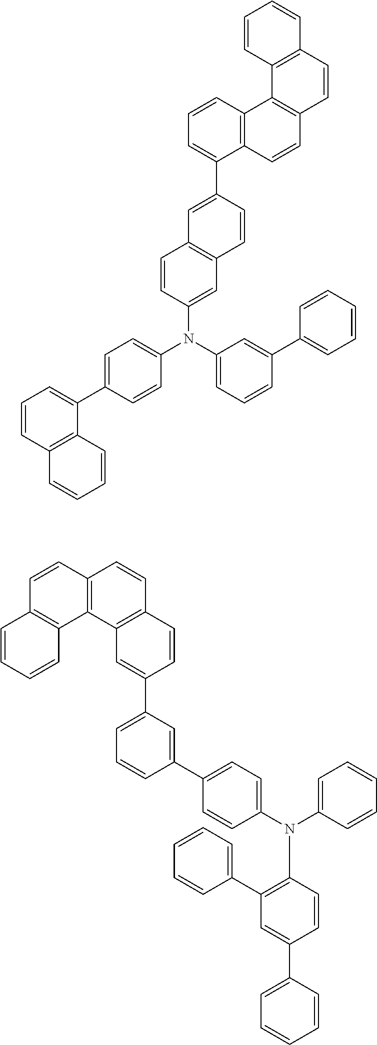 [SMILES: c1ccc(-c2cccc(N(c3ccc(-c4cccc5ccccc45)cc3)c3ccc4cc(-c5cccc6c5ccc5ccc7ccccc7c56)ccc4c3)c2)cc1 ; c1ccc(-c2ccc(N(c3ccccc3)c3ccc(-c4cccc(-c5ccc6ccc7ccc8ccccc8c7c6c5)c4)cc3)c(-c3ccccc3)c2)cc1]

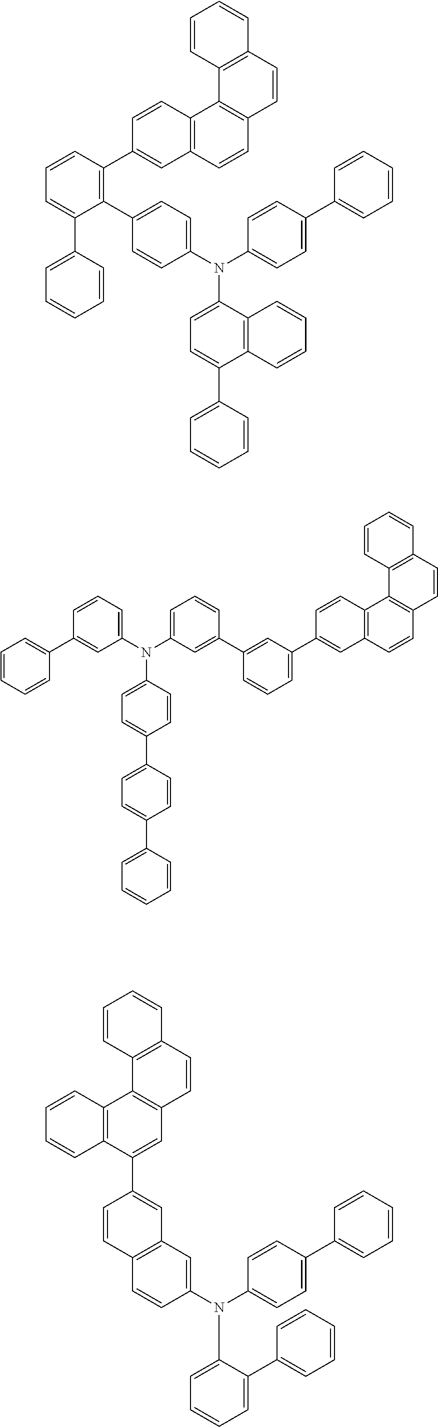 [SMILES: c1ccc(-c2ccc(N(c3ccc(-c4c(-c5ccccc5)cccc4-c4ccc5c(ccc6ccc7ccccc7c65)c4)cc3)c3ccc(-c4ccccc4)c4ccccc34)cc2)cc1 ; c1ccc(-c2ccc(-c3ccc(N(c4cccc(-c5ccccc5)c4)c4cccc(-c5cccc(-c6ccc7c(ccc8ccc9ccccc9c87)c6)c5)c4)cc3)cc2)cc1 ; c1ccc(-c2ccc(N(c3ccc4ccc(-c5cc6ccc7ccccc7c6c6ccccc56)cc4c3)c3ccccc3-c3ccccc3)cc2)cc1]

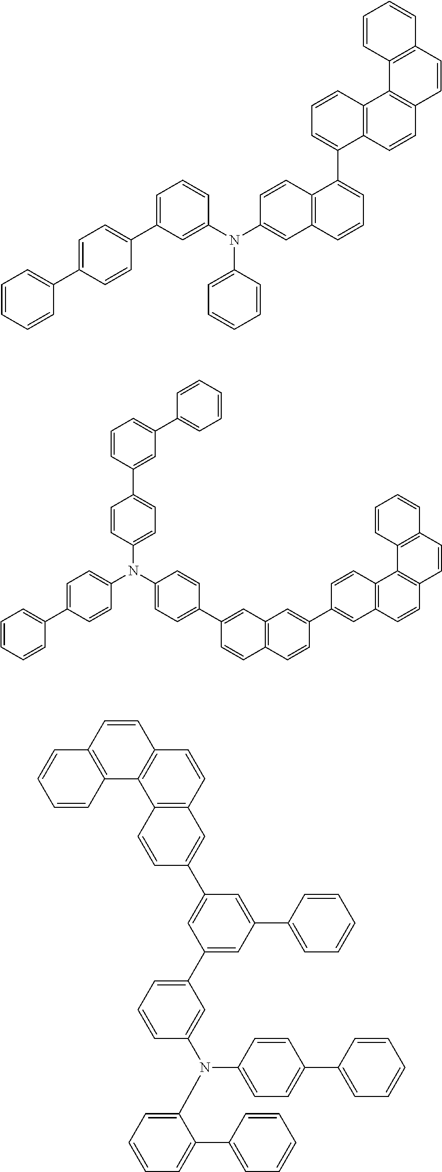 [SMILES: c1ccc(-c2ccc(-c3cccc(N(c4ccccc4)c4ccc5c(-c6cccc7c6ccc6ccc8ccccc8c67)cccc5c4)c3)cc2)cc1 ; c1ccc(-c2ccc(N(c3ccc(-c4cccc(-c5ccccc5)c4)cc3)c3ccc(-c4ccc5ccc(-c6ccc7c(ccc8ccc9ccccc9c87)c6)cc5c4)cc3)cc2)cc1 ; c1ccc(-c2ccc(N(c3cccc(-c4cc(-c5ccccc5)cc(-c5ccc6c(ccc7ccc8ccccc8c76)c5)c4)c3)c3ccccc3-c3ccccc3)cc2)cc1]

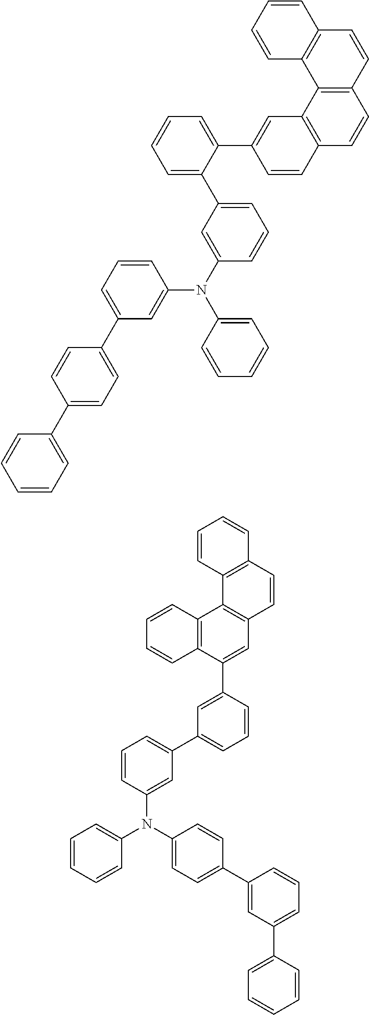 [SMILES: c1ccc(-c2ccc(-c3cccc(N(c4ccccc4)c4cccc(-c5ccccc5-c5ccc6ccc7ccc8ccccc8c7c6c5)c4)c3)cc2)cc1 ; c1ccc(-c2cccc(-c3ccc(N(c4ccccc4)c4cccc(-c5cccc(-c6cc7ccc8ccccc8c7c7ccccc67)c5)c4)cc3)c2)cc1]

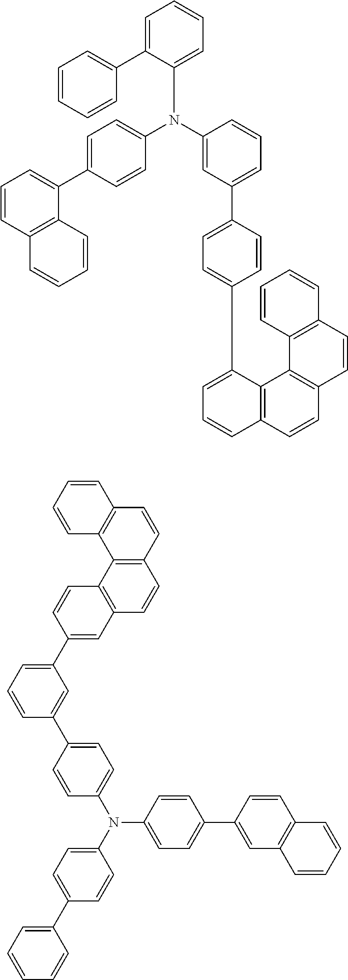 [SMILES: c1ccc(-c2ccccc2N(c2ccc(-c3cccc4ccccc34)cc2)c2cccc(-c3ccc(-c4cccc5ccc6ccc7ccccc7c6c45)cc3)c2)cc1 ; c1ccc(-c2ccc(N(c3ccc(-c4cccc(-c5ccc6c(ccc7ccc8ccccc8c76)c5)c4)cc3)c3ccc(-c4ccc5ccccc5c4)cc3)cc2)cc1]

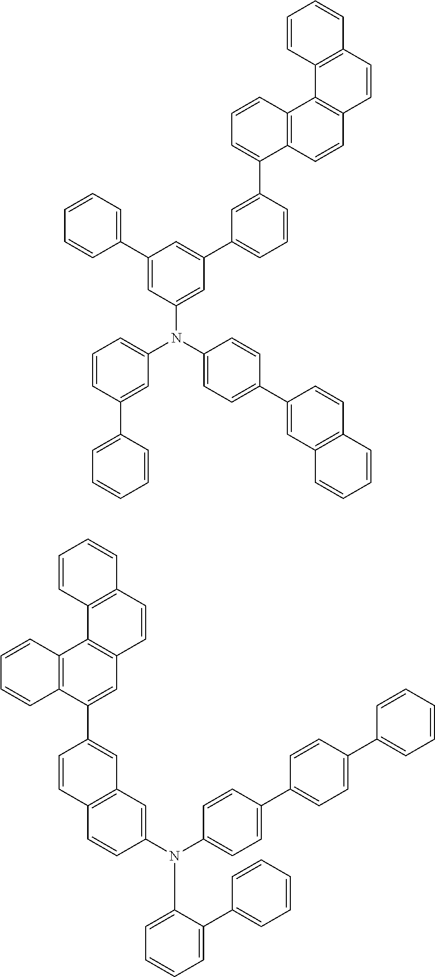 [SMILES: c1ccc(-c2cccc(N(c3ccc(-c4ccc5ccccc5c4)cc3)c3cc(-c4ccccc4)cc(-c4cccc(-c5cccc6c5ccc5ccc7ccccc7c56)c4)c3)c2)cc1 ; c1ccc(-c2ccc(-c3ccc(N(c4ccc5ccc(-c6cc7ccc8ccccc8c7c7ccccc67)cc5c4)c4ccccc4-c4ccccc4)cc3)cc2)cc1]

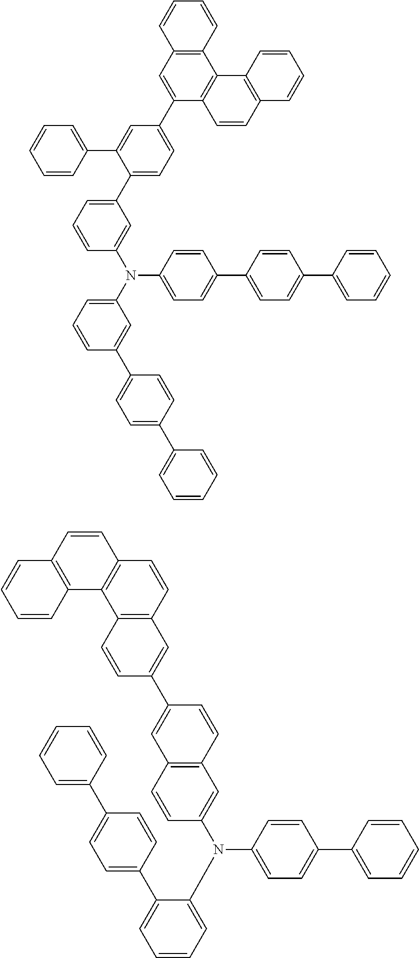 [SMILES: c1ccc(-c2ccc(-c3ccc(N(c4cccc(-c5ccc(-c6ccccc6)cc5)c4)c4cccc(-c5ccc(-c6cc7ccccc7c7c6ccc6ccccc67)cc5-c5ccccc5)c4)cc3)cc2)cc1 ; c1ccc(-c2ccc(-c3ccccc3N(c3ccc(-c4ccccc4)cc3)c3ccc4cc(-c5ccc6c(ccc7ccc8ccccc8c76)c5)ccc4c3)cc2)cc1]

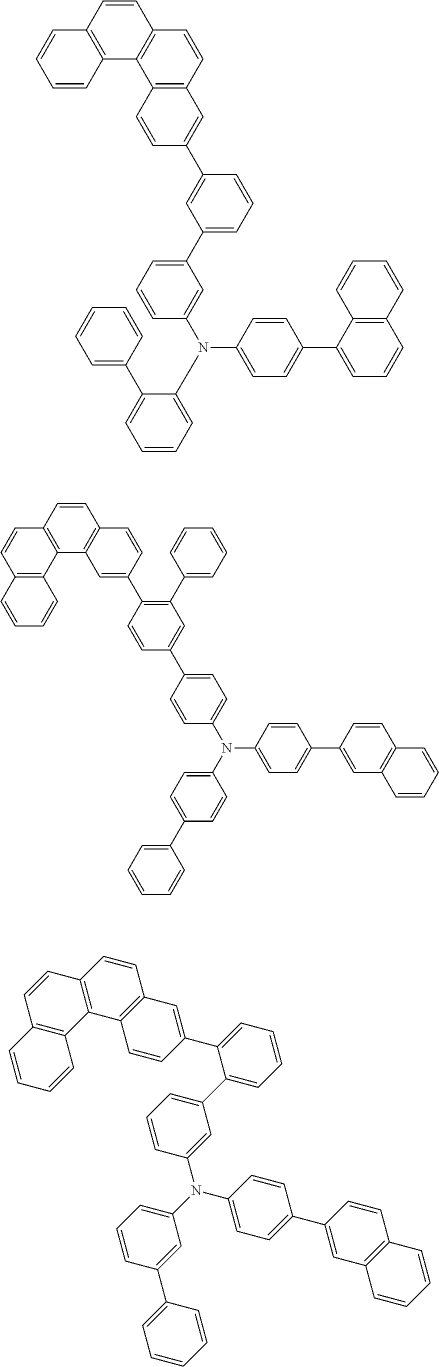 [SMILES: c1ccc(-c2ccccc2N(c2ccc(-c3cccc4ccccc34)cc2)c2cccc(-c3cccc(-c4ccc5c(ccc6ccc7ccccc7c65)c4)c3)c2)cc1 ; c1ccc(-c2ccc(N(c3ccc(-c4ccc(-c5ccc6ccc7ccc8ccccc8c7c6c5)c(-c5ccccc5)c4)cc3)c3ccc(-c4ccc5ccccc5c4)cc3)cc2)cc1 ; c1ccc(-c2cccc(N(c3ccc(-c4ccc5ccccc5c4)cc3)c3cccc(-c4ccccc4-c4ccc5c(ccc6ccc7ccccc7c65)c4)c3)c2)cc1]

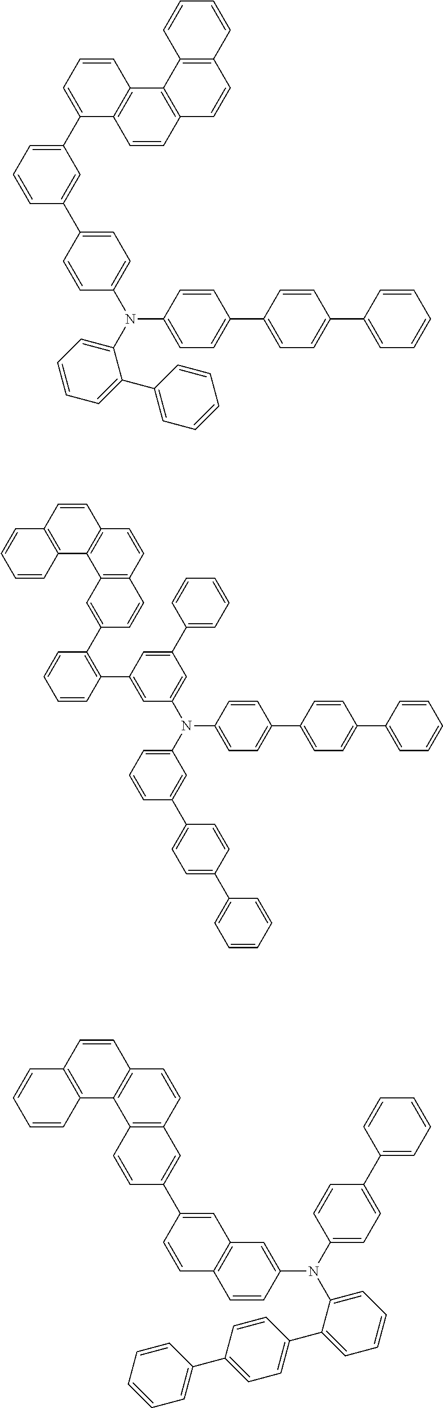 [SMILES: c1ccc(-c2ccc(-c3ccc(N(c4ccc(-c5cccc(-c6cccc7c6ccc6ccc8ccccc8c67)c5)cc4)c4ccccc4-c4ccccc4)cc3)cc2)cc1 ; c1ccc(-c2ccc(-c3ccc(N(c4cccc(-c5ccc(-c6ccccc6)cc5)c4)c4cc(-c5ccccc5)cc(-c5ccccc5-c5ccc6ccc7ccc8ccccc8c7c6c5)c4)cc3)cc2)cc1 ; c1ccc(-c2ccc(-c3ccccc3N(c3ccc(-c4ccccc4)cc3)c3ccc4ccc(-c5ccc6c(ccc7ccc8ccccc8c76)c5)cc4c3)cc2)cc1]

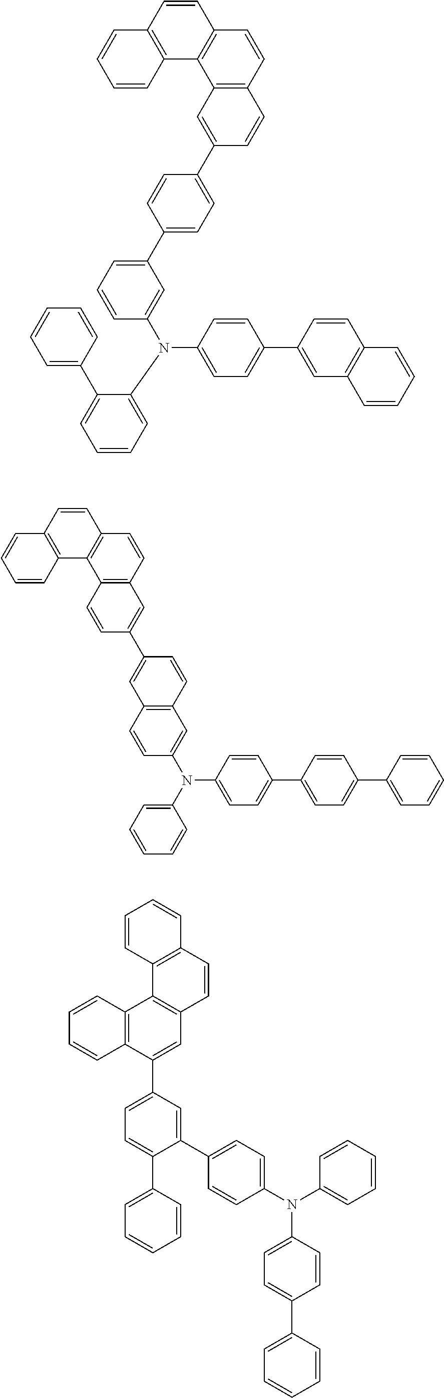 [SMILES: c1ccc(-c2ccccc2N(c2ccc(-c3ccc4ccccc4c3)cc2)c2cccc(-c3ccc(-c4ccc5ccc6ccc7ccccc7c6c5c4)cc3)c2)cc1 ; c1ccc(-c2ccc(-c3ccc(N(c4ccccc4)c4ccc5cc(-c6ccc7c(ccc8ccc9ccccc9c87)c6)ccc5c4)cc3)cc2)cc1 ; c1ccc(-c2ccc(N(c3ccccc3)c3ccc(-c4cc(-c5cc6ccc7ccccc7c6c6ccccc56)ccc4-c4ccccc4)cc3)cc2)cc1]

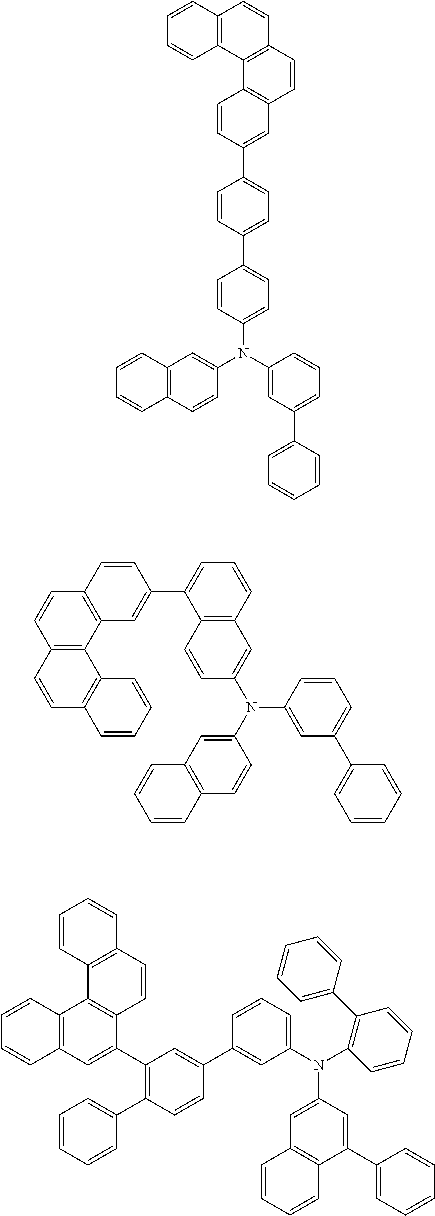 [SMILES: c1ccc(-c2cccc(N(c3ccc(-c4ccc(-c5ccc6c(ccc7ccc8ccccc8c76)c5)cc4)cc3)c3ccc4ccccc4c3)c2)cc1 ; c1ccc(-c2cccc(N(c3ccc4ccccc4c3)c3ccc4c(-c5ccc6ccc7ccc8ccccc8c7c6c5)cccc4c3)c2)cc1 ; c1ccc(-c2ccc(-c3cccc(N(c4cc(-c5ccccc5)c5ccccc5c4)c4ccccc4-c4ccccc4)c3)cc2-c2cc3ccccc3c3c2ccc2ccccc23)cc1]

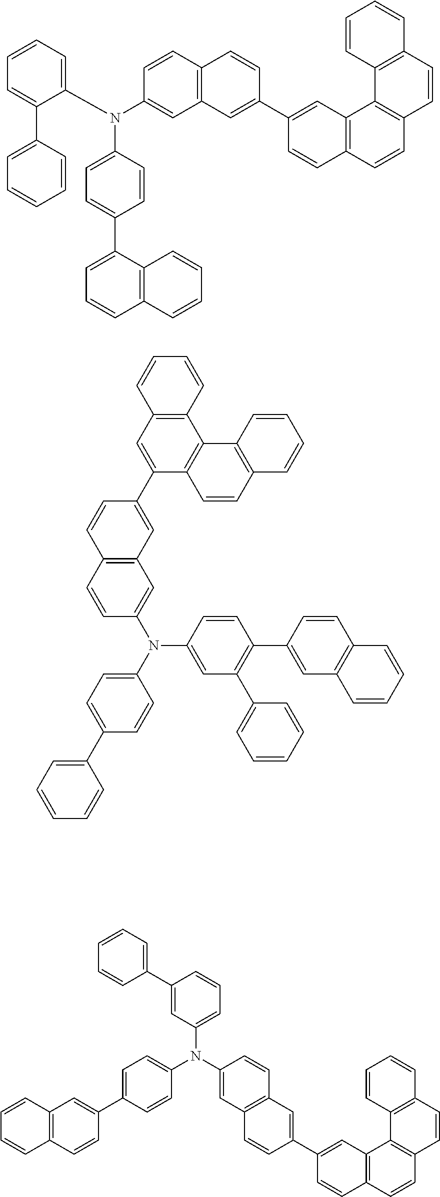

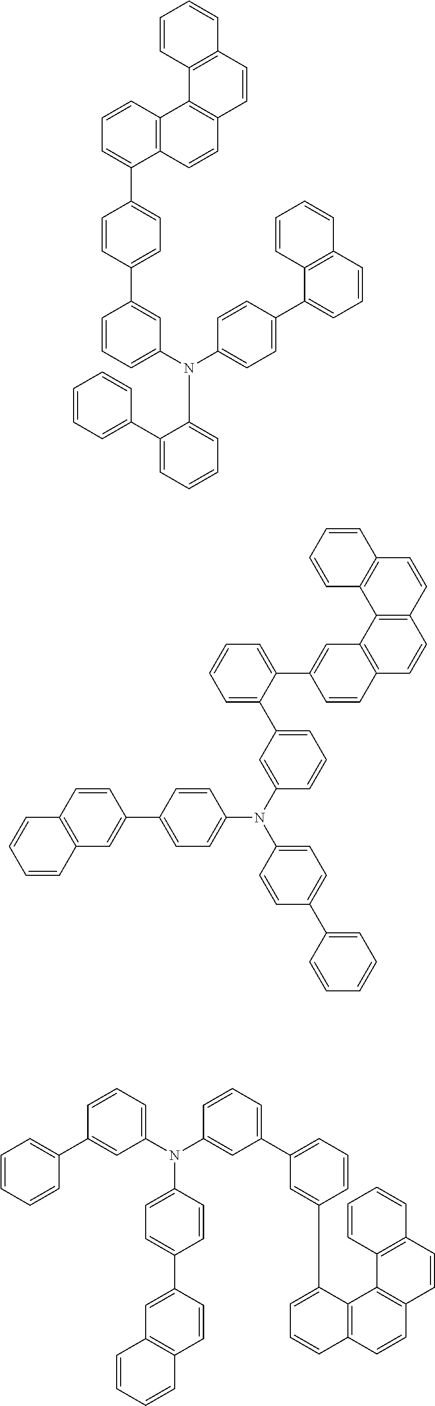 [SMILES: c1ccc(-c2ccccc2N(c2ccc(-c3cccc4ccccc34)cc2)c2cccc(-c3ccc(-c4cccc5c4ccc4ccc6ccccc6c45)cc3)c2)cc1 ; c1ccc(-c2ccc(N(c3ccc(-c4ccc5ccccc5c4)cc3)c3cccc(-c4ccccc4-c4ccc5ccc6ccc7ccccc7c6c5c4)c3)cc2)cc1 ; c1ccc(-c2cccc(N(c3ccc(-c4ccc5ccccc5c4)cc3)c3cccc(-c4cccc(-c5cccc6ccc7ccc8ccccc8c7c56)c4)c3)c2)cc1]

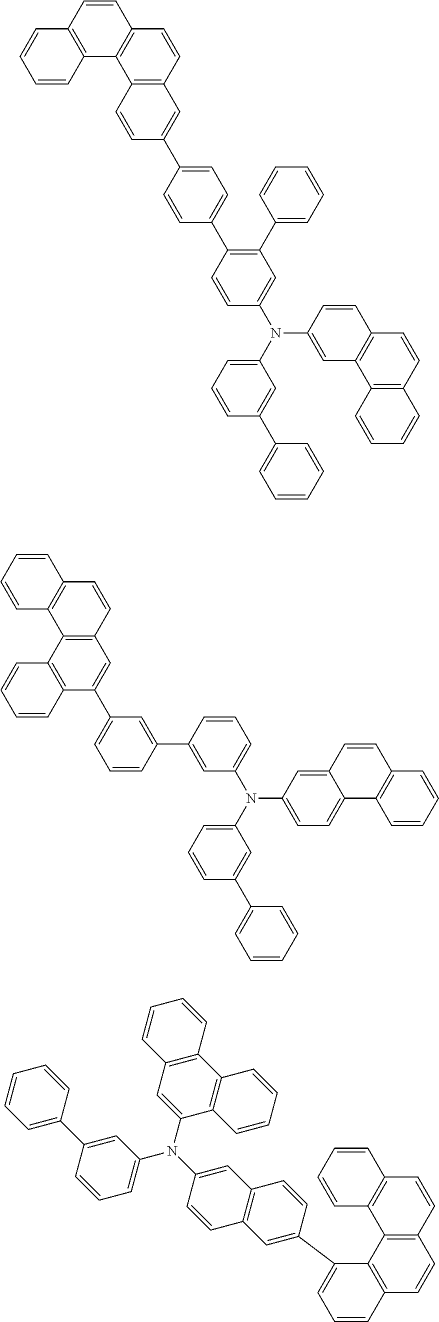 [SMILES: c1ccc(-c2cccc(N(c3ccc(-c4ccc(-c5ccc6c(ccc7ccc8ccccc8c76)c5)cc4)c(-c4ccccc4)c3)c3ccc4ccc5ccccc5c4c3)c2)cc1 ; c1ccc(-c2cccc(N(c3cccc(-c4cccc(-c5cc6ccc7ccccc7c6c6ccccc56)c4)c3)c3ccc4c(ccc5ccccc54)c3)c2)cc1 ; c1ccc(-c2cccc(N(c3ccc4cc(-c5cccc6ccc7ccc8ccccc8c7c56)ccc4c3)c3cc4ccccc4c4ccccc34)c2)cc1]

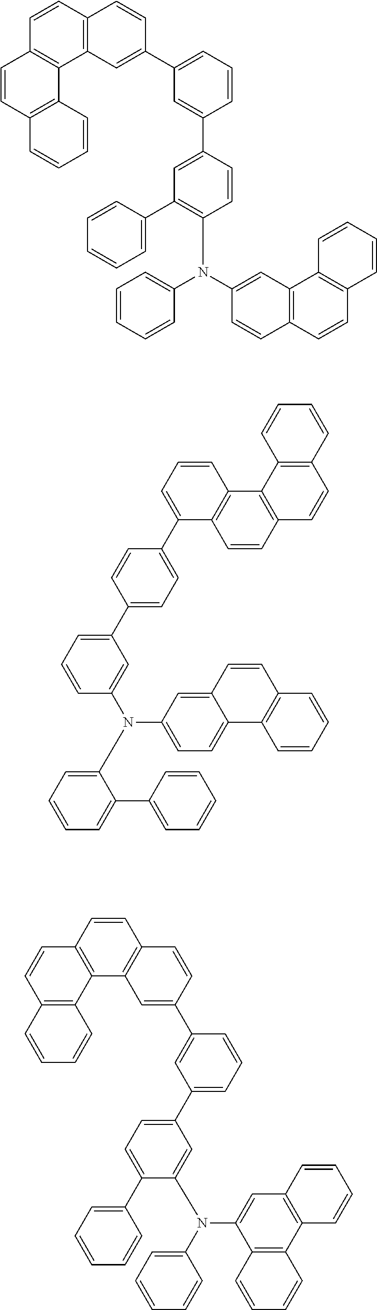 [SMILES: c1ccc(-c2cc(-c3cccc(-c4ccc5ccc6ccc7ccccc7c6c5c4)c3)ccc2N(c2ccccc2)c2ccc3ccc4ccccc4c3c2)cc1 ; c1ccc(-c2ccccc2N(c2cccc(-c3ccc(-c4cccc5c4ccc4ccc6ccccc6c45)cc3)c2)c2ccc3c(ccc4ccccc43)c2)cc1 ; c1ccc(-c2ccc(-c3cccc(-c4ccc5ccc6ccc7ccccc7c6c5c4)c3)cc2N(c2ccccc2)c2cc3ccccc3c3ccccc23)cc1]

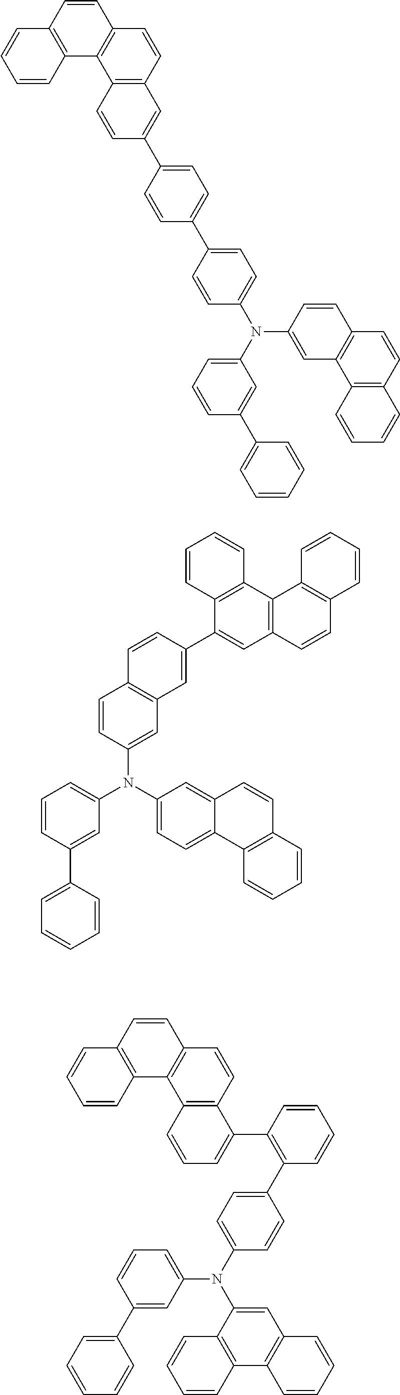 [SMILES: c1ccc(-c2cccc(N(c3ccc(-c4ccc(-c5ccc6c(ccc7ccc8ccccc8c76)c5)cc4)cc3)c3ccc4ccc5ccccc5c4c3)c2)cc1 ; c1ccc(-c2cccc(N(c3ccc4ccc(-c5cc6ccc7ccccc7c6c6ccccc56)cc4c3)c3ccc4c(ccc5ccccc54)c3)c2)cc1 ; c1ccc(-c2cccc(N(c3ccc(-c4ccccc4-c4cccc5c4ccc4ccc6ccccc6c45)cc3)c3cc4ccccc4c4ccccc34)c2)cc1]

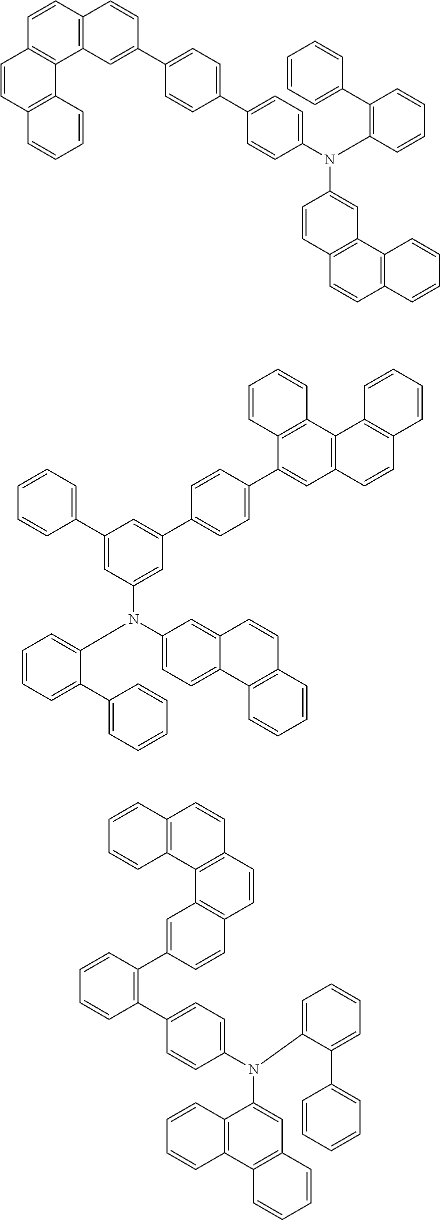 [SMILES: c1ccc(-c2ccccc2N(c2ccc(-c3ccc(-c4ccc5ccc6ccc7ccccc7c6c5c4)cc3)cc2)c2ccc3ccc4ccccc4c3c2)cc1 ; c1ccc(-c2cc(-c3ccc(-c4cc5ccc6ccccc6c5c5ccccc45)cc3)cc(N(c3ccc4c(ccc5ccccc54)c3)c3ccccc3-c3ccccc3)c2)cc1 ; c1ccc(-c2ccccc2N(c2ccc(-c3ccccc3-c3ccc4ccc5ccc6ccccc6c5c4c3)cc2)c2cc3ccccc3c3ccccc23)cc1]

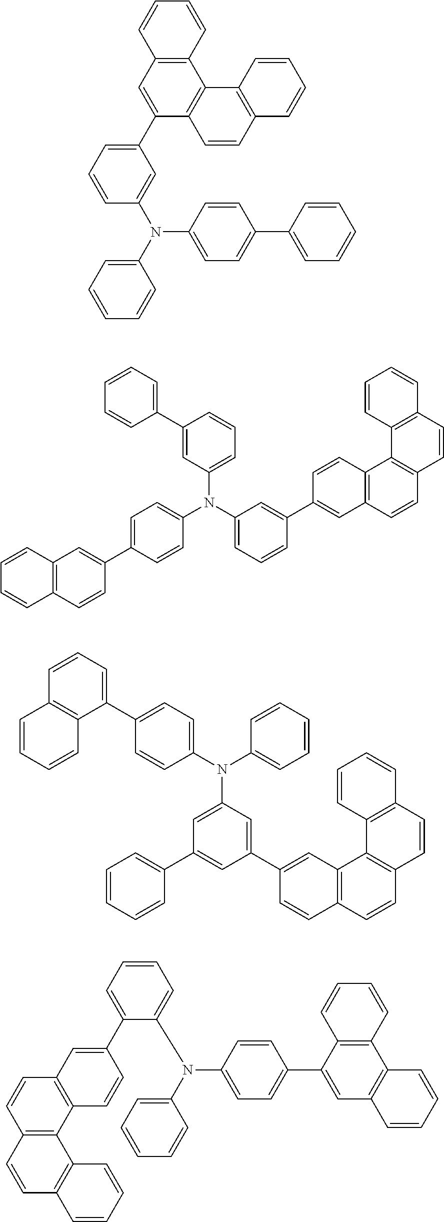

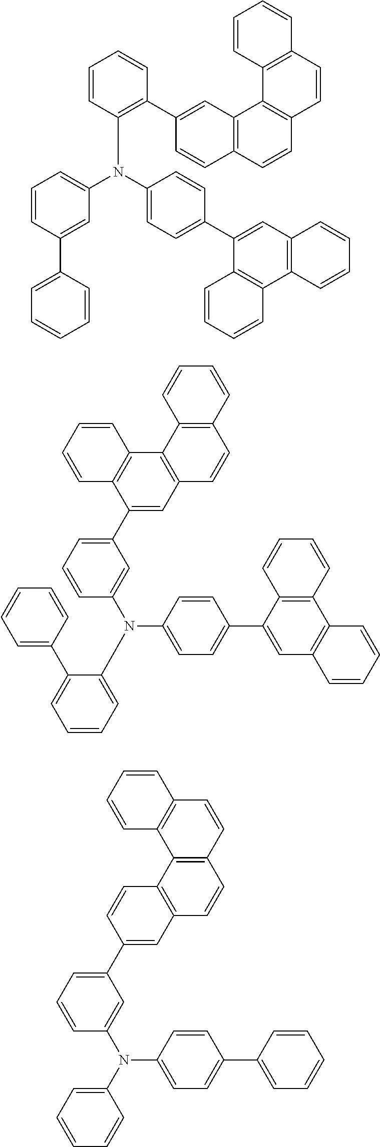

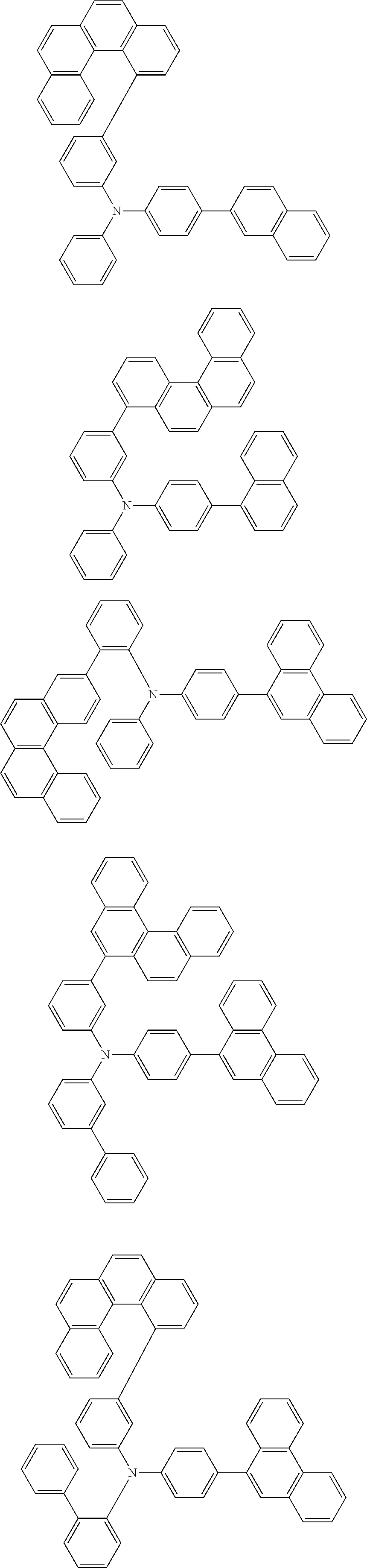

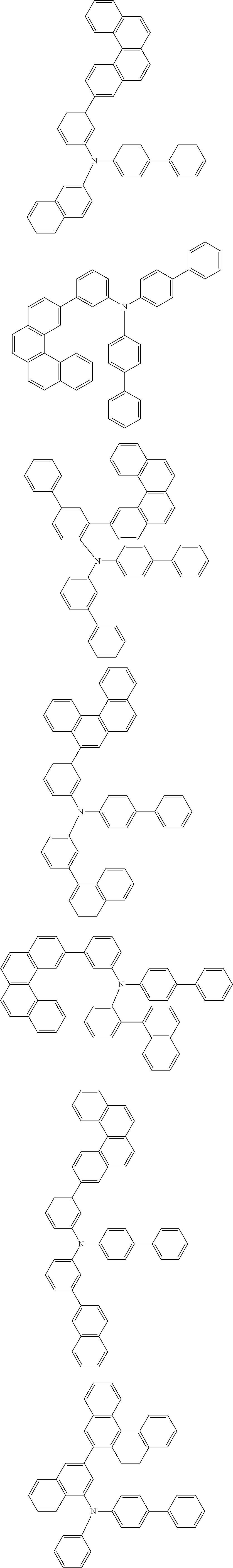

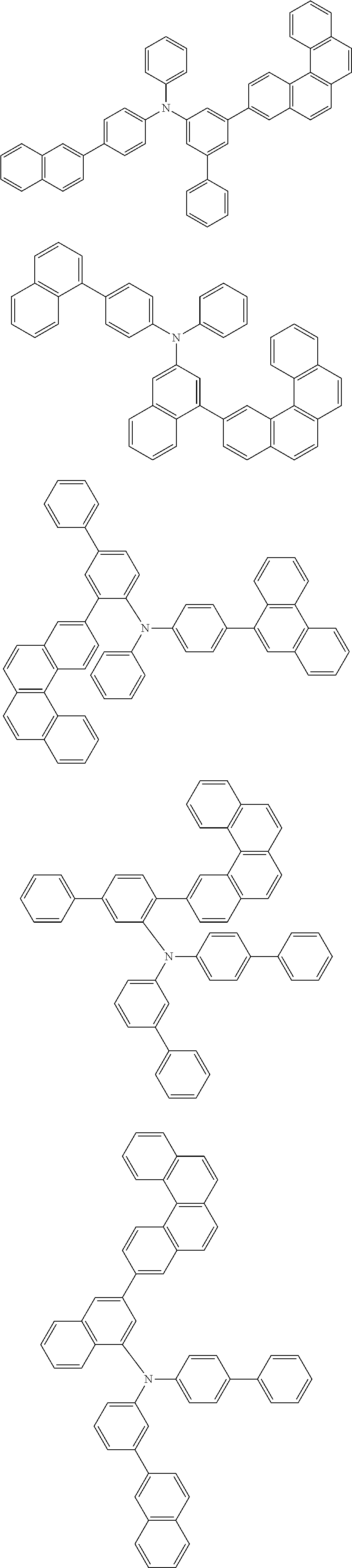

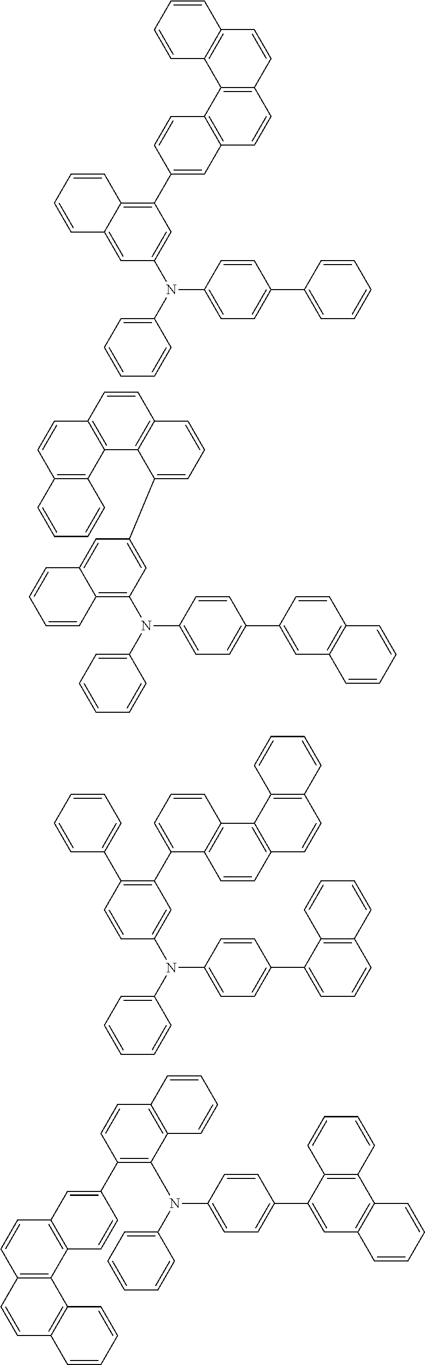

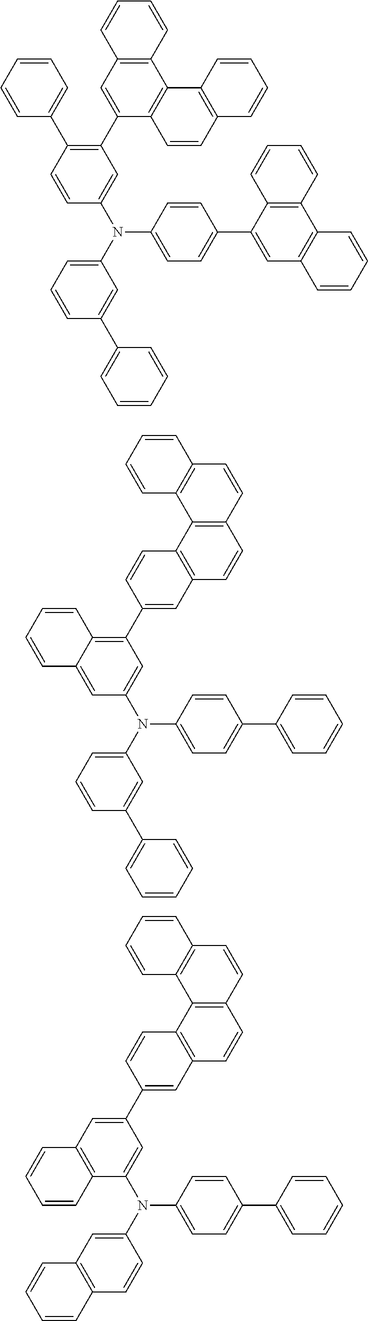

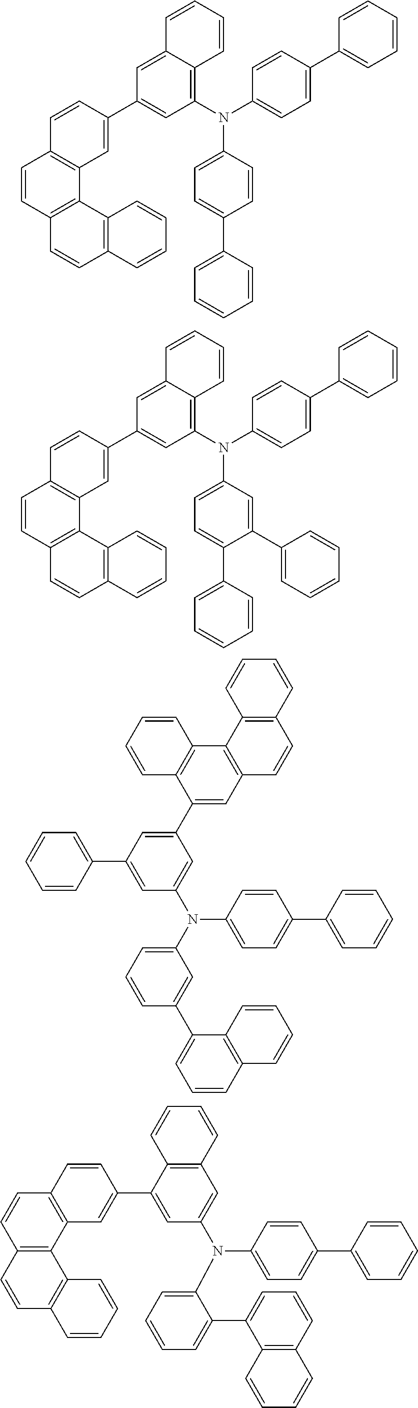

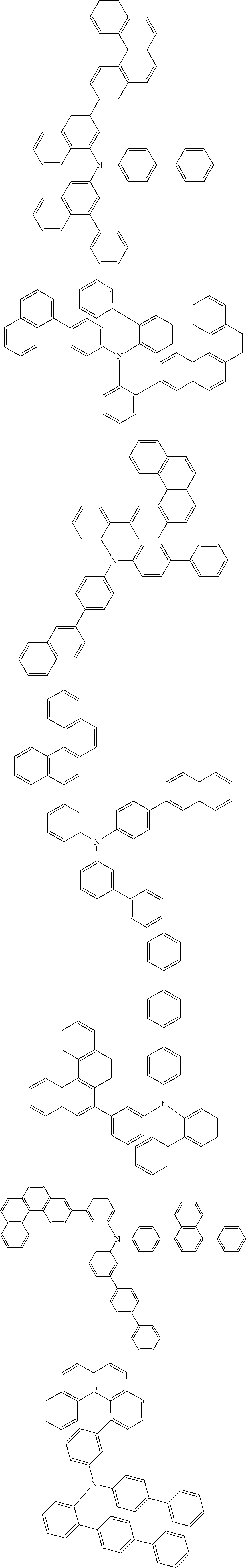

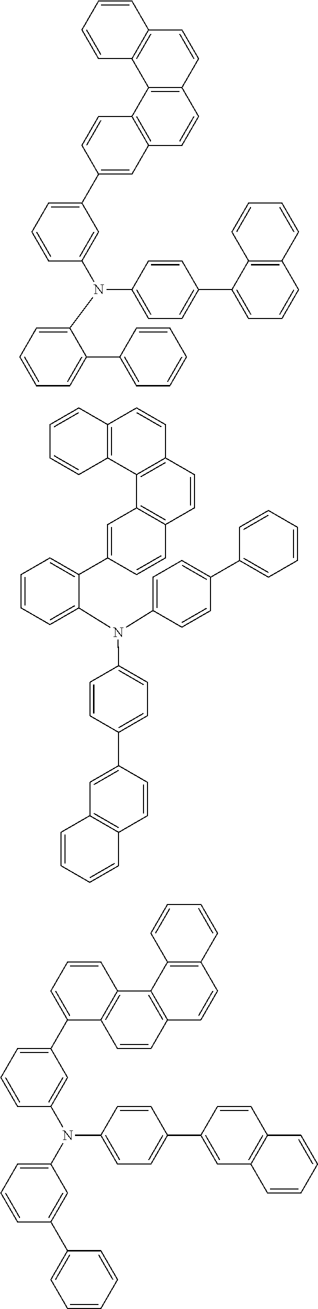

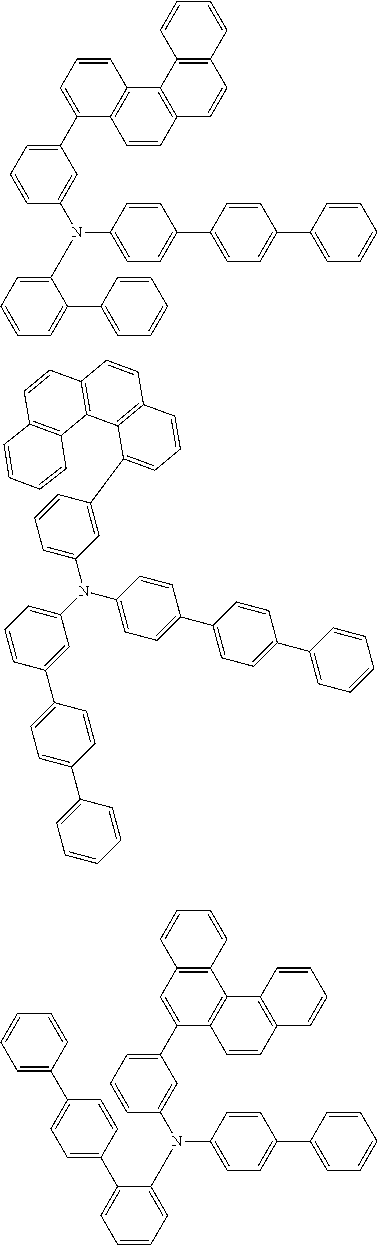

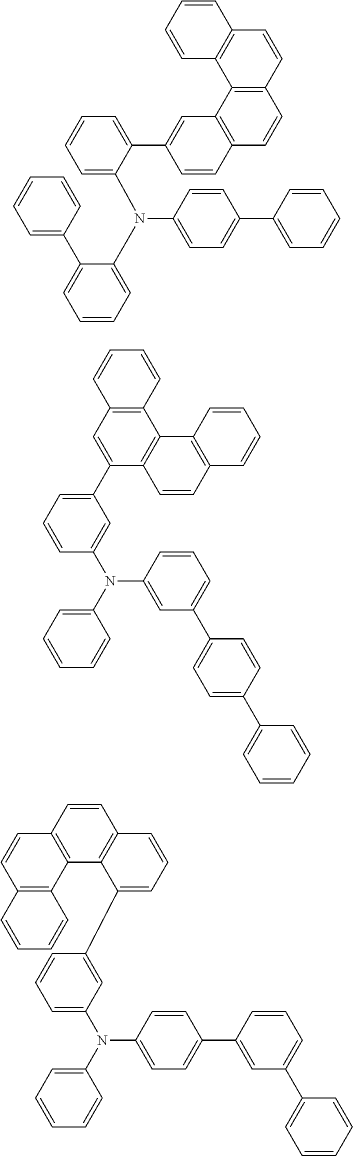

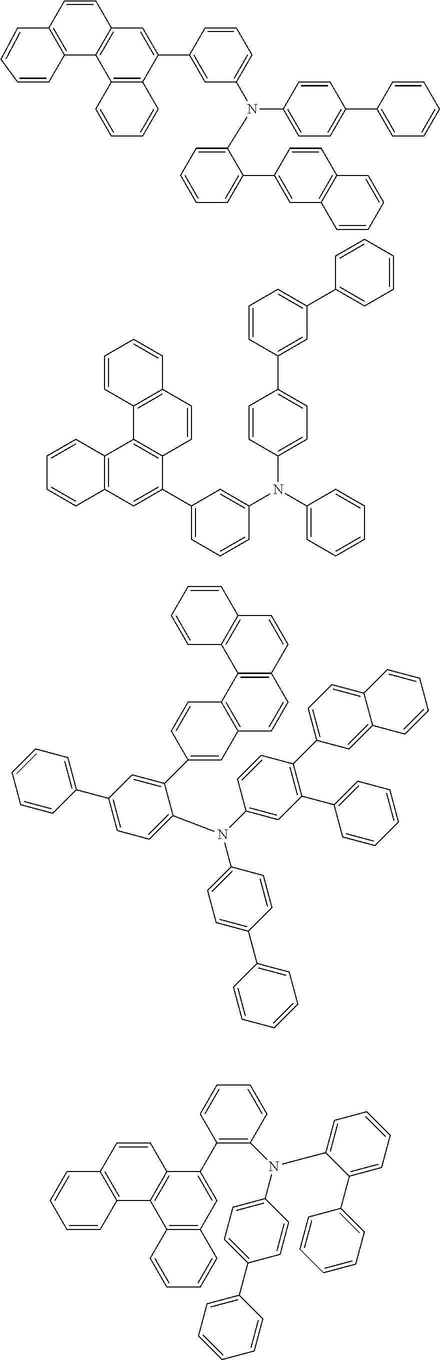

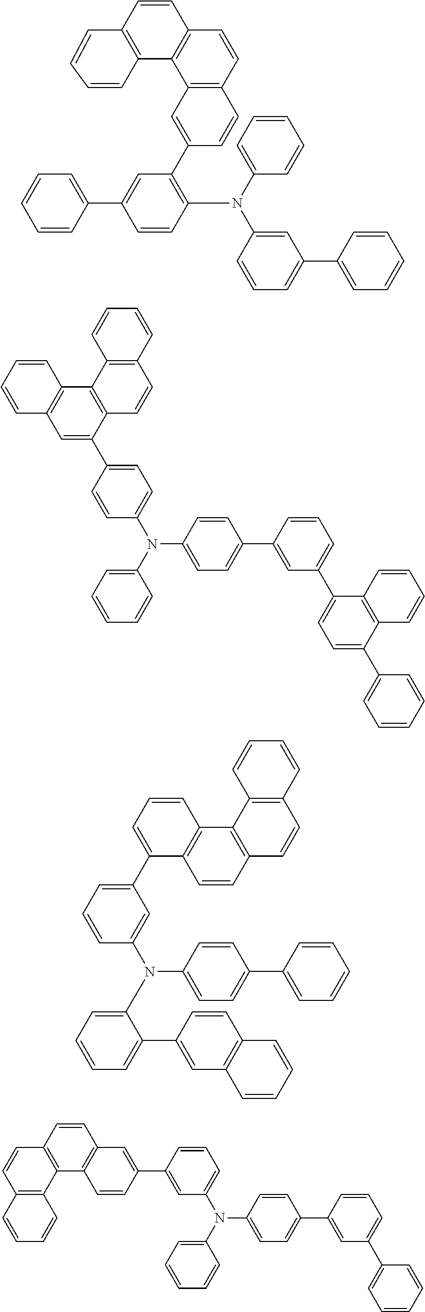 [SMILES: c1ccc(-c2cccc(N(c3ccccc3)c3ccc(-c4ccccc4)cc3-c3ccc4ccc5ccc6ccccc6c5c4c3)c2)cc1 ; c1ccc(-c2ccc(-c3cccc(-c4ccc(N(c5ccccc5)c5ccc(-c6cc7ccccc7c7c6ccc6ccccc67)cc5)cc4)c3)c3ccccc23)cc1 ; c1ccc(-c2ccc(N(c3cccc(-c4cccc5c4ccc4ccc6ccccc6c45)c3)c3ccccc3-c3ccc4ccccc4c3)cc2)cc1 ; c1ccc(-c2cccc(-c3ccc(N(c4ccccc4)c4cccc(-c5ccc6c(ccc7ccc8ccccc8c76)c5)c4)cc3)c2)cc1]

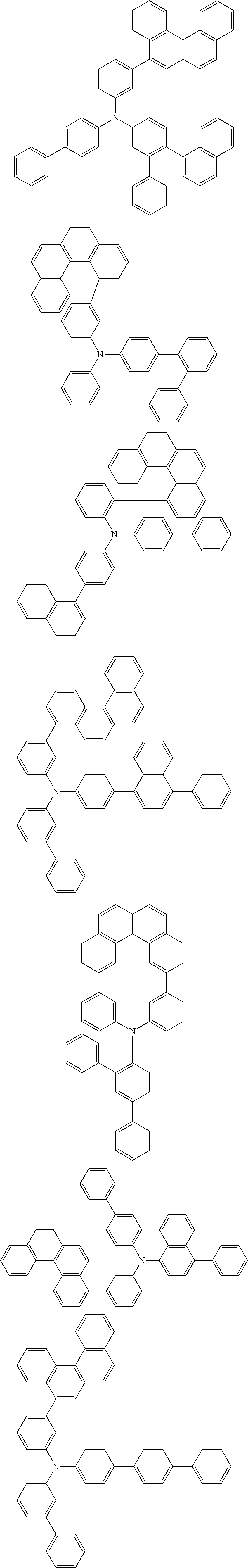

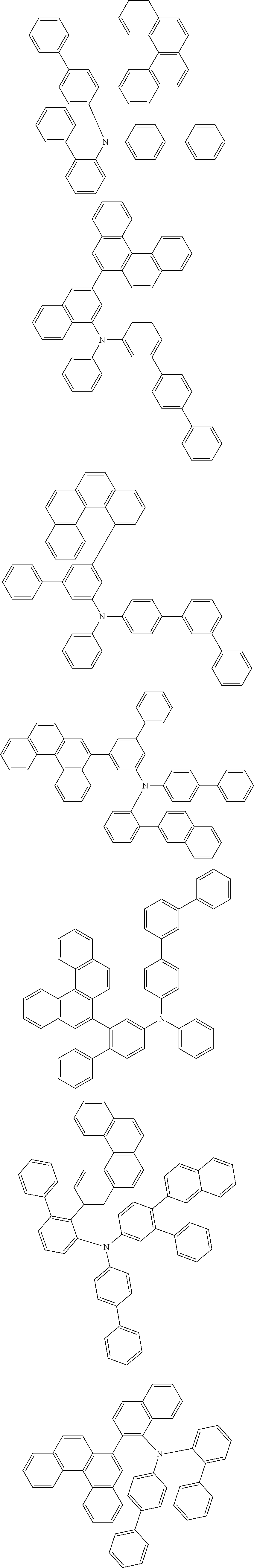

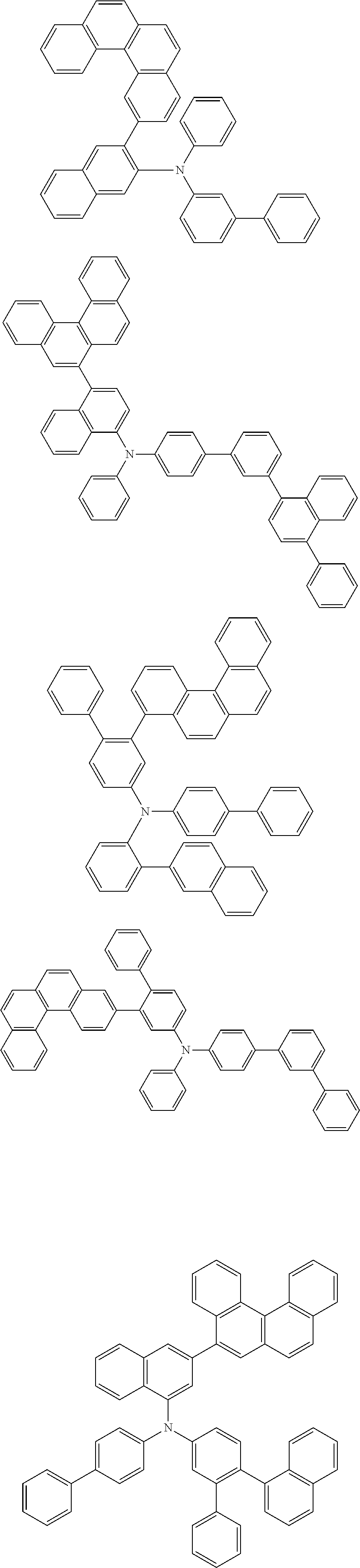 [SMILES: c1ccc(-c2cccc(N(c3ccccc3)c3cc4ccccc4cc3-c3ccc4ccc5ccc6ccccc6c5c4c3)c2)cc1 ; c1ccc(-c2ccc(-c3cccc(-c4ccc(N(c5ccccc5)c5ccc(-c6cc7ccccc7c7c6ccc6ccccc67)c6ccccc56)cc4)c3)c3ccccc23)cc1 ; c1ccc(-c2ccc(N(c3ccc(-c4ccccc4)c(-c4cccc5c4ccc4ccc6ccccc6c45)c3)c3ccccc3-c3ccc4ccccc4c3)cc2)cc1 ; c1ccc(-c2cccc(-c3ccc(N(c4ccccc4)c4ccc(-c5ccccc5)c(-c5ccc6c(ccc7ccc8ccccc8c76)c5)c4)cc3)c2)cc1 ; c1ccc(-c2ccc(N(c3ccc(-c4cccc5ccccc45)c(-c4ccccc4)c3)c3cc(-c4cc5ccc6ccccc6c5c5ccccc45)cc4ccccc34)cc2)cc1]

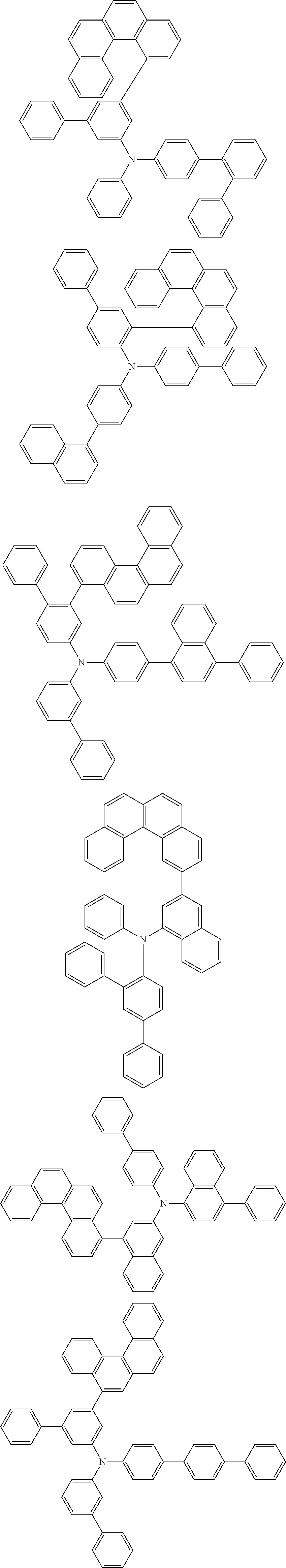

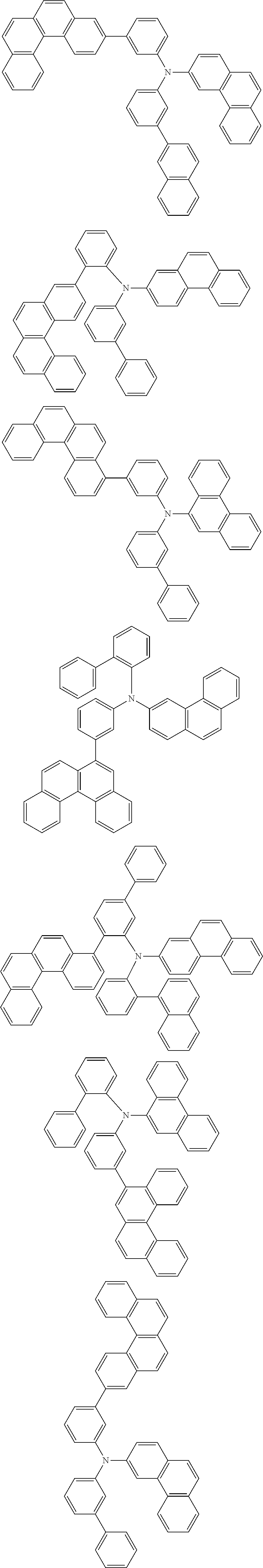

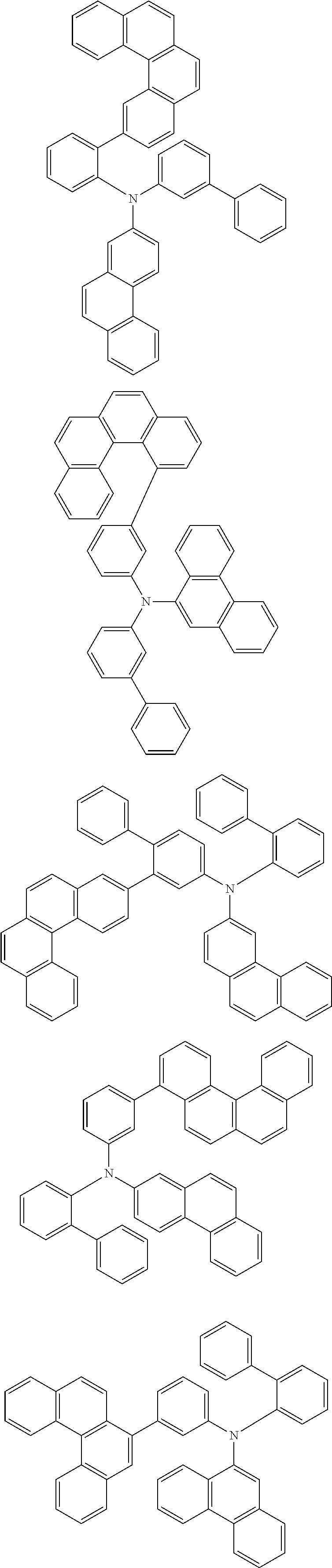

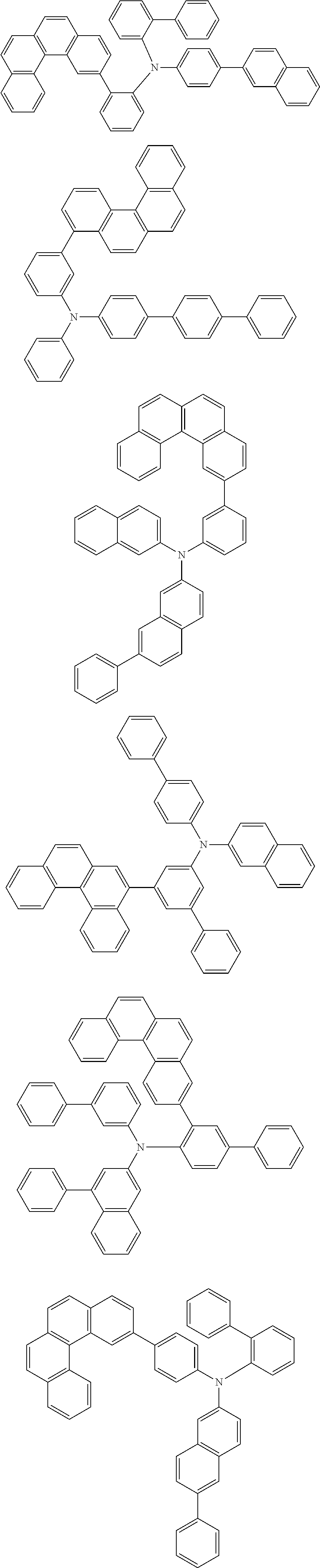

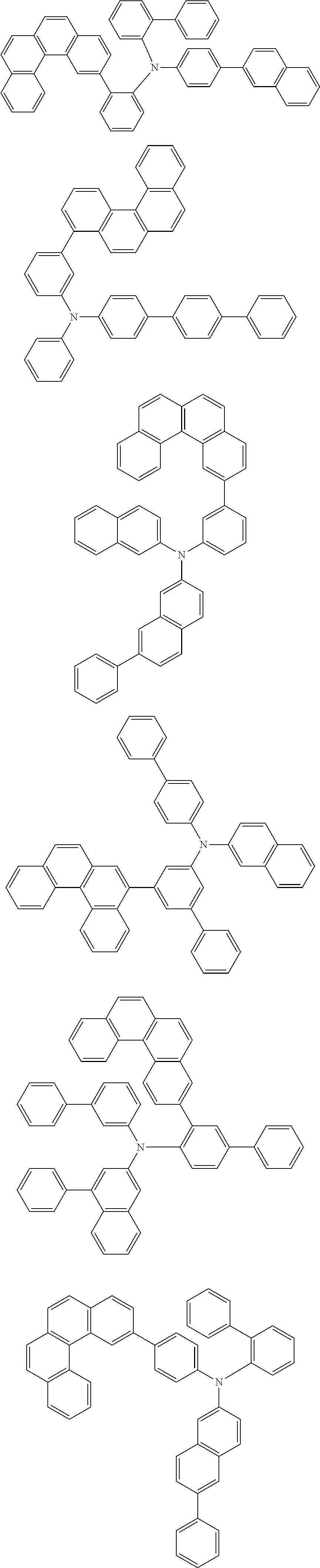

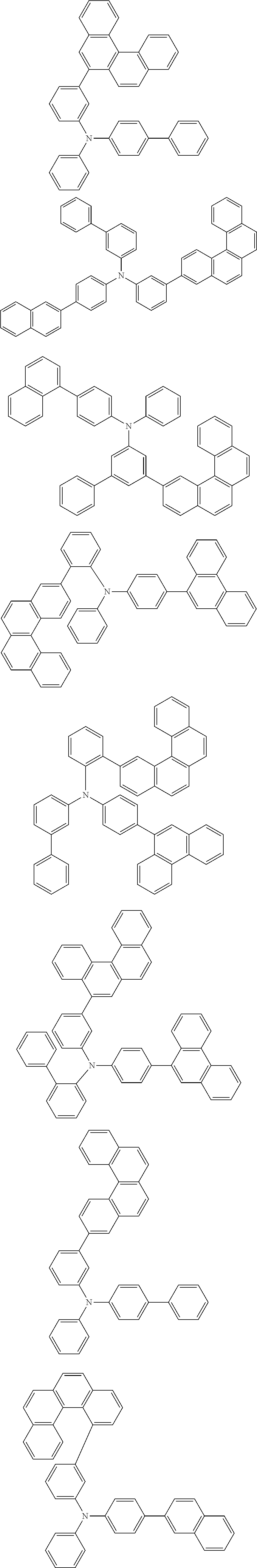

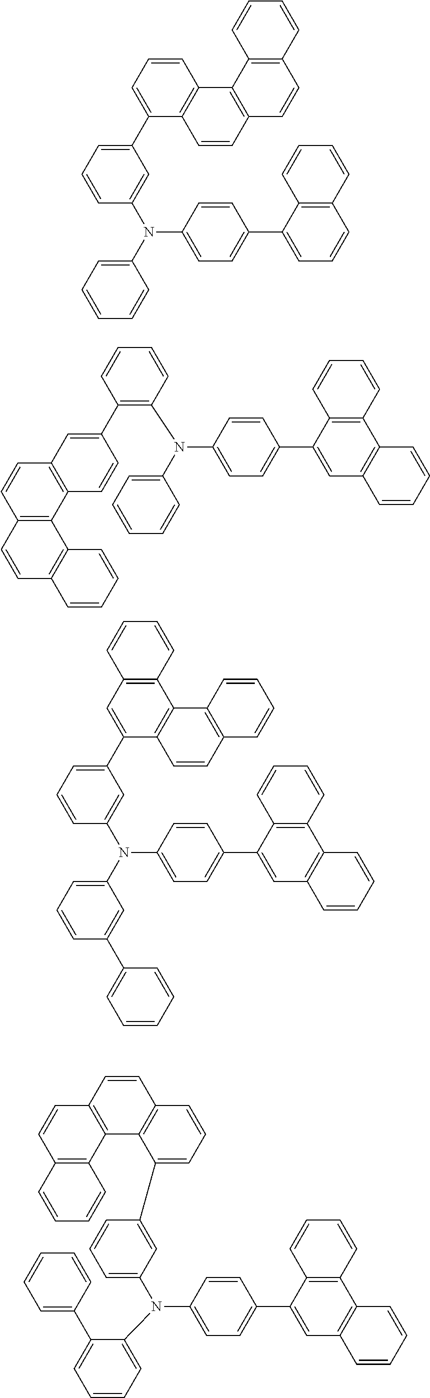

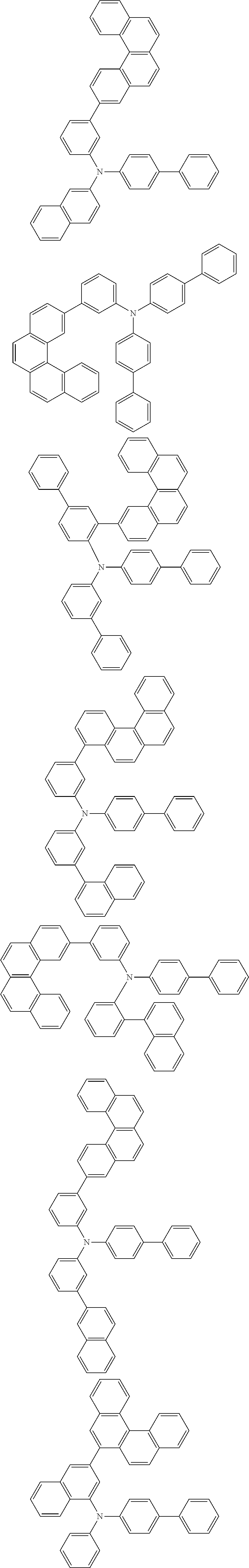

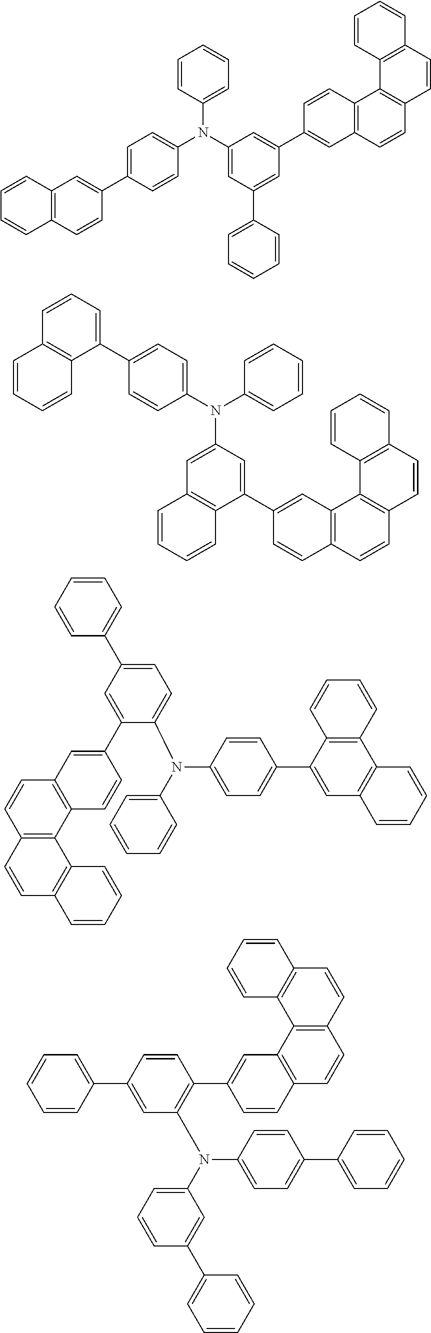 [SMILES: c1ccc(-c2cc(-c3ccc4c(ccc5ccc6ccccc6c54)c3)cc(N(c3ccccc3)c3ccc(-c4ccc5ccccc5c4)cc3)c2)cc1 ; c1ccc(N(c2ccc(-c3cccc4ccccc34)cc2)c2cc(-c3ccc4ccc5ccc6ccccc6c5c4c3)c3ccccc3c2)cc1 ; c1ccc(-c2ccc(N(c3ccccc3)c3ccc(-c4cc5ccccc5c5ccccc45)cc3)c(-c3ccc4c(ccc5ccc6ccccc6c54)c3)c2)cc1 ; c1ccc(-c2ccc(N(c3cccc(-c4ccccc4)c3)c3cc(-c4ccccc4)ccc3-c3ccc4ccc5ccc6ccccc6c5c4c3)cc2)cc1]

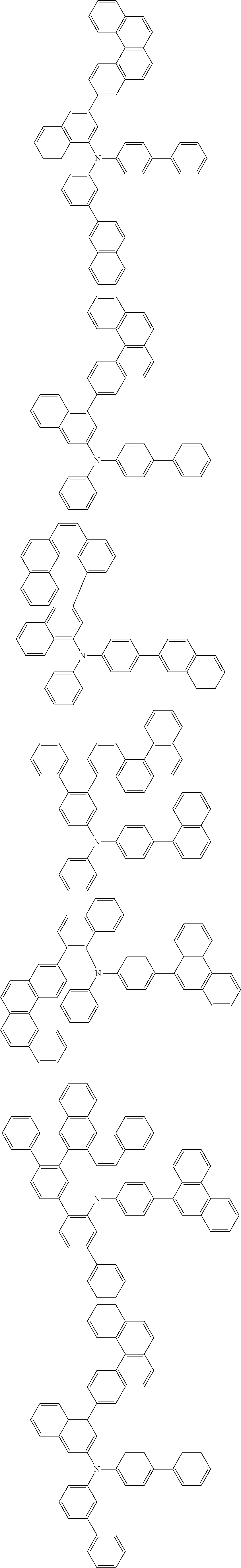

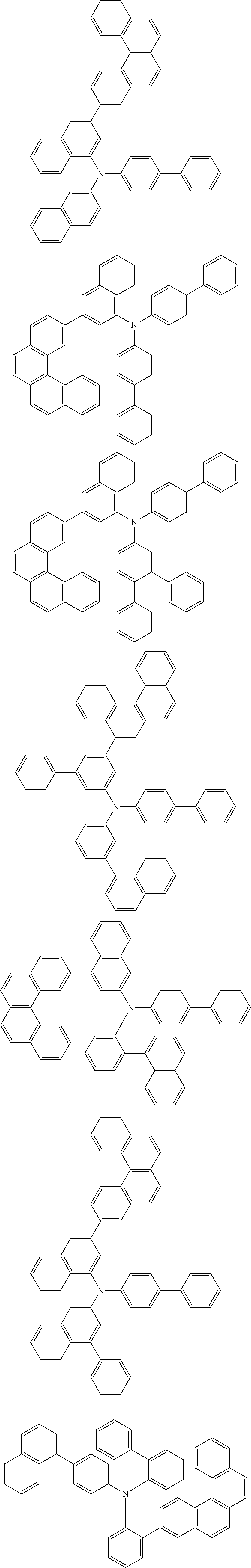

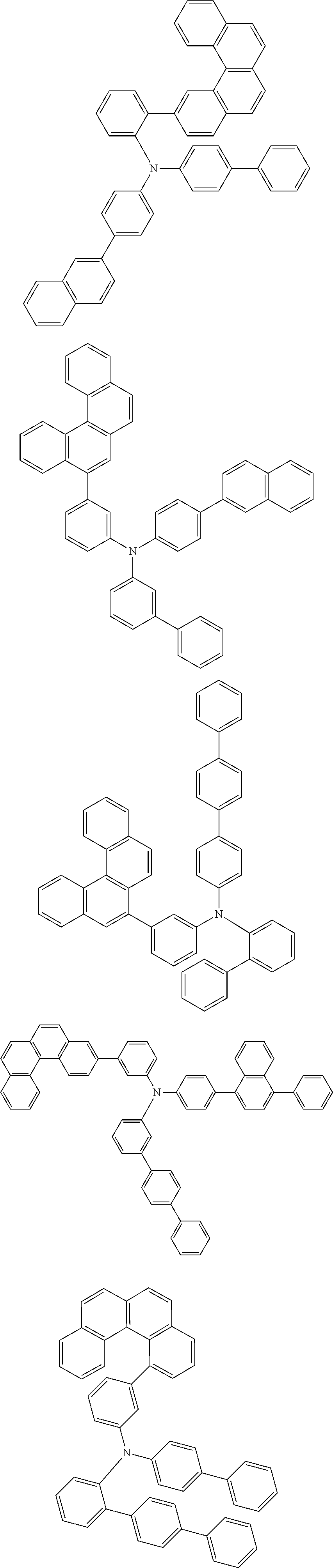

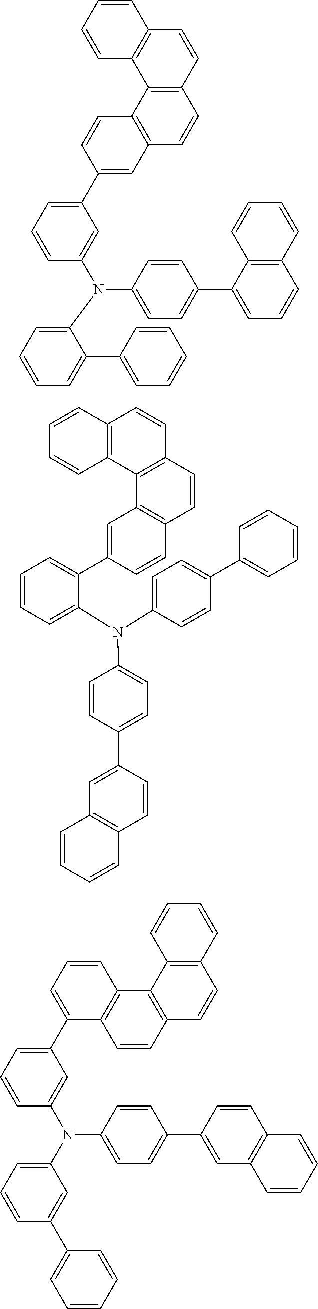

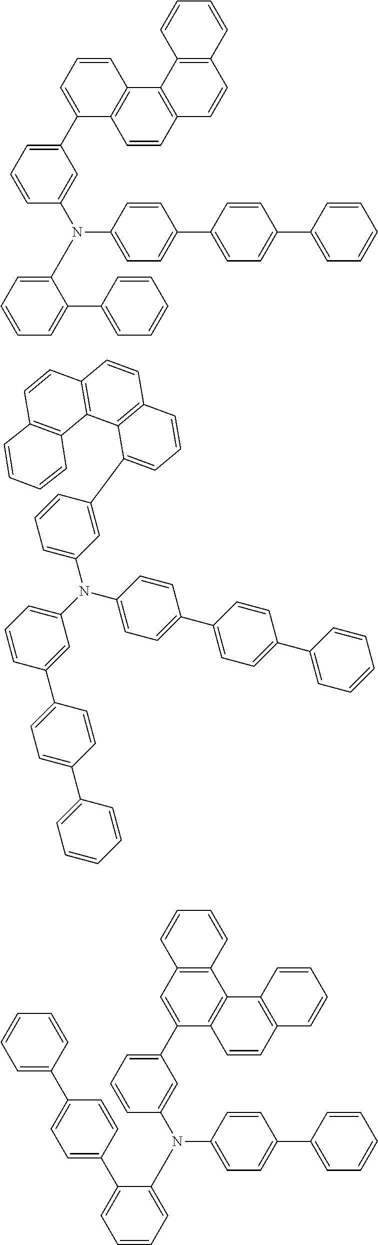

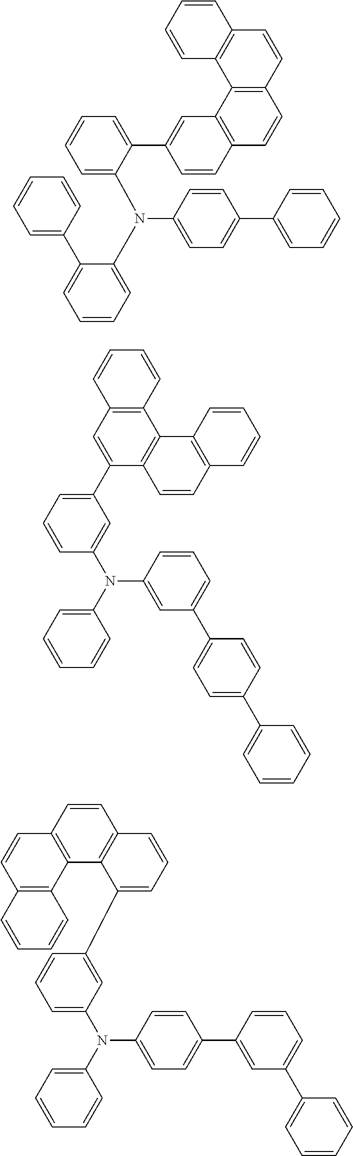

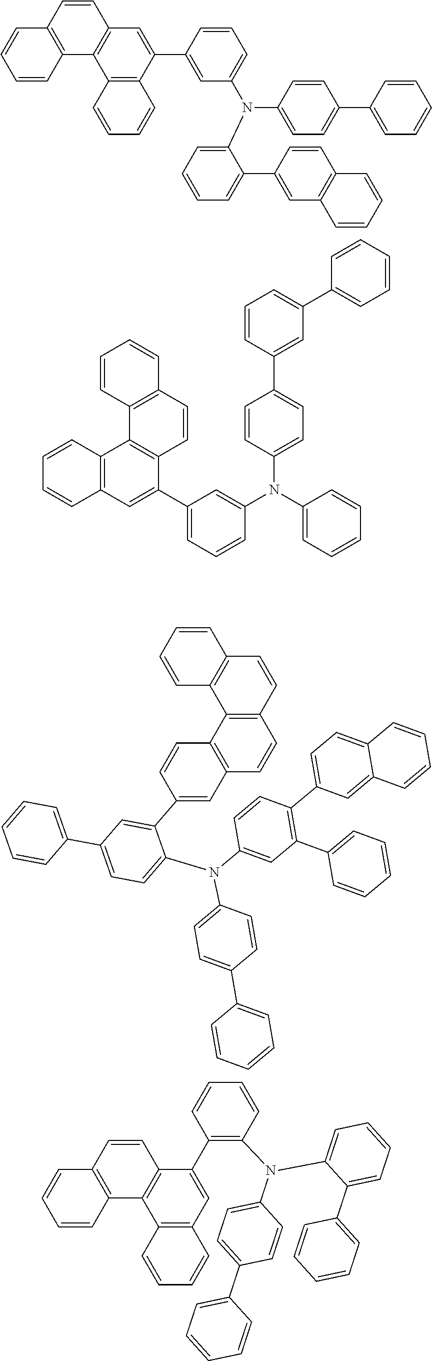

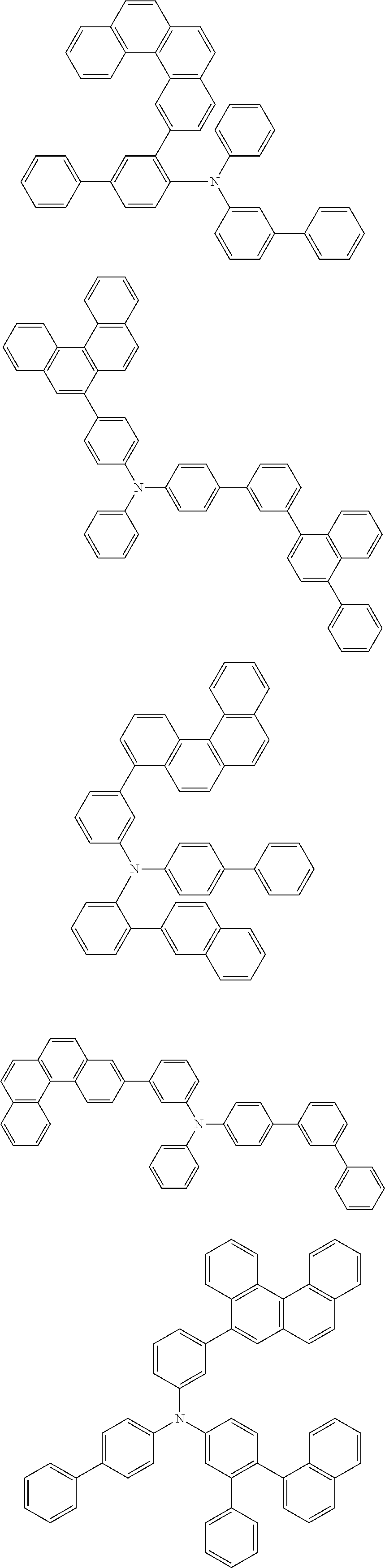

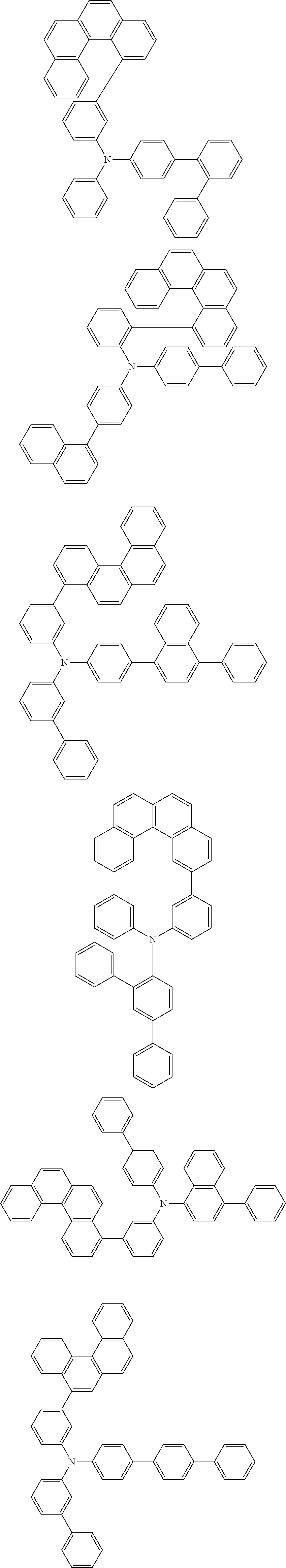

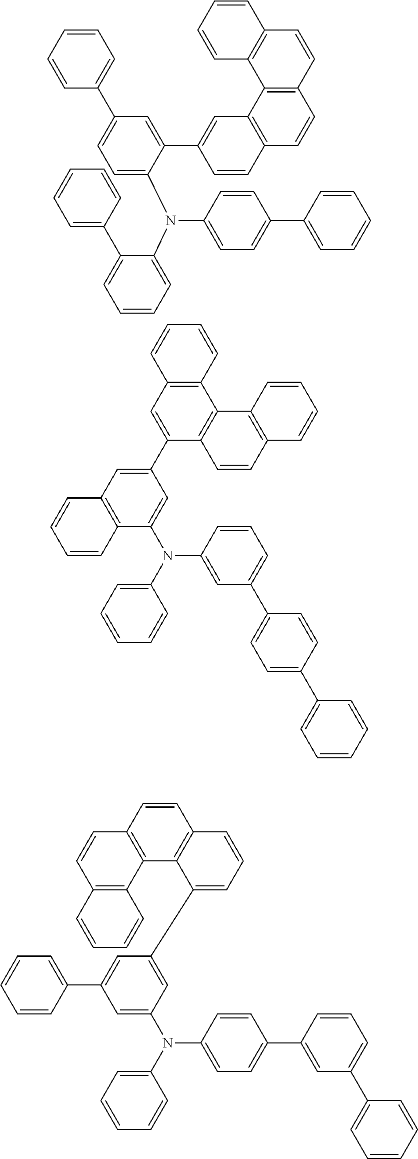

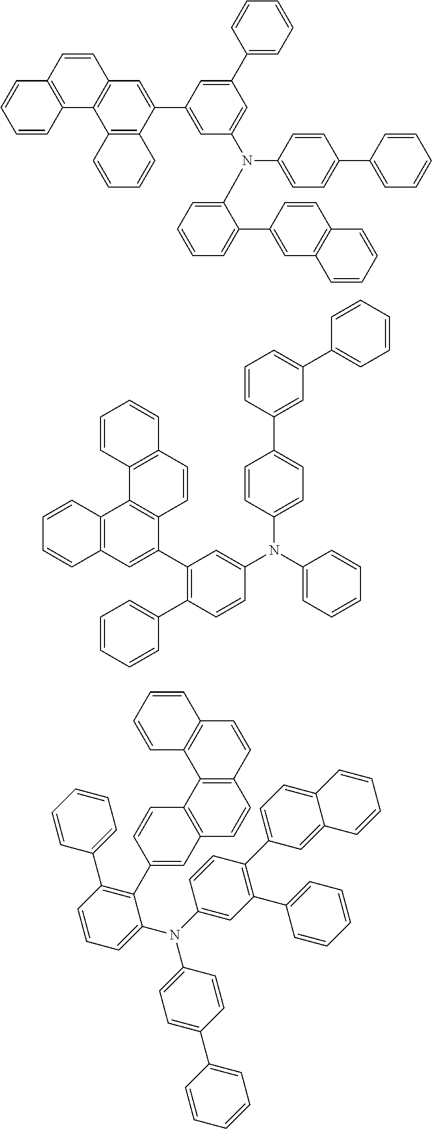

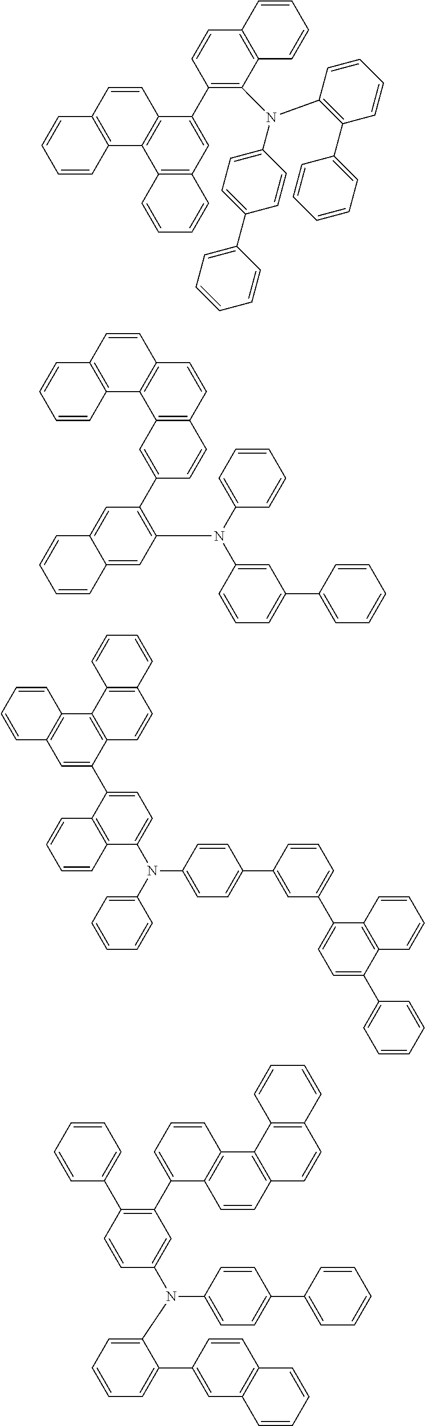

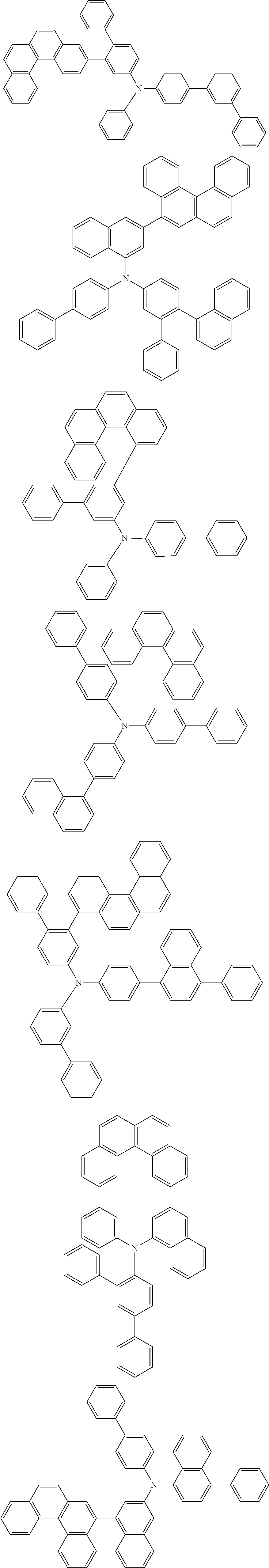

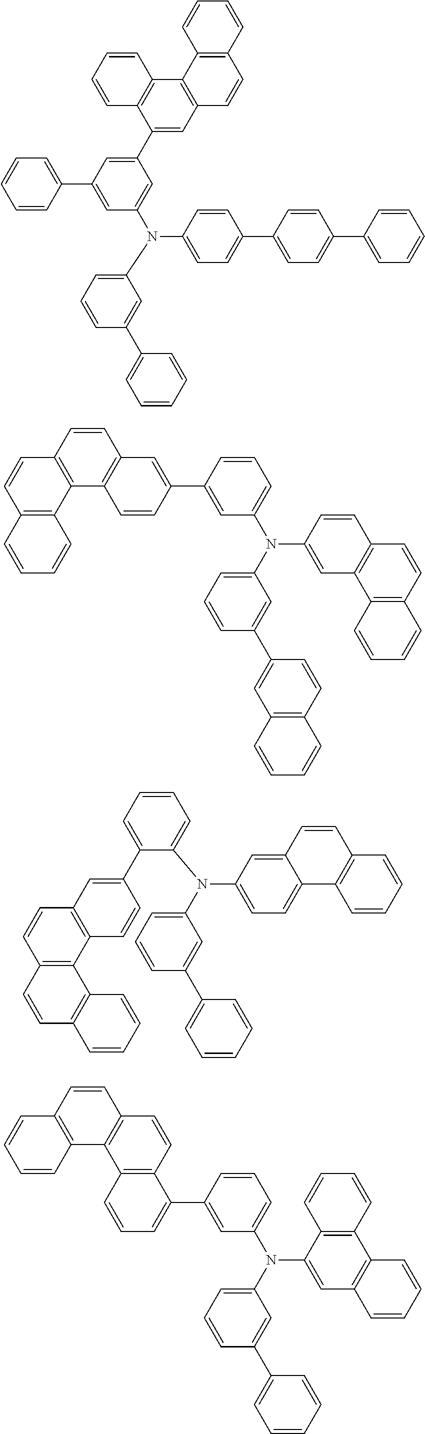

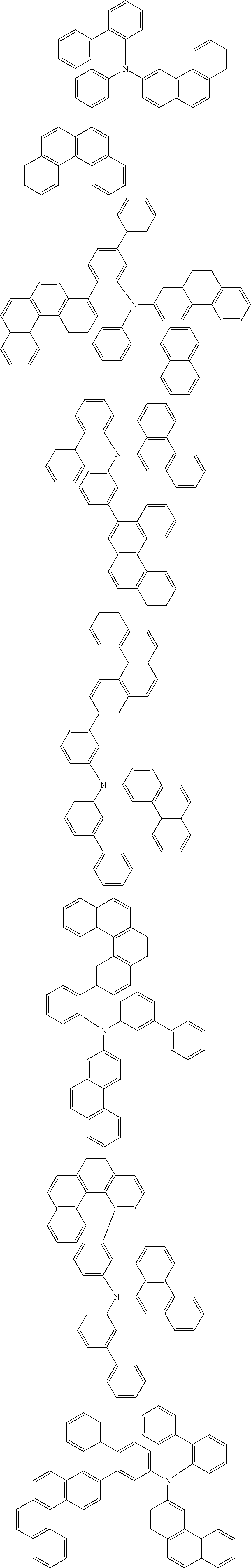

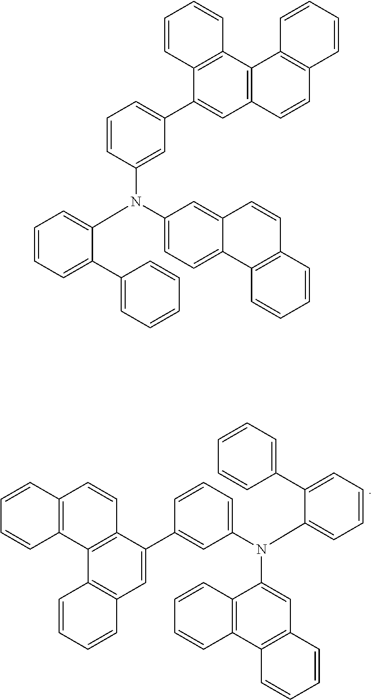 [SMILES: c1ccc(-c2ccccc2N(c2cccc(-c3cc4ccc5ccccc5c4c4ccccc34)c2)c2ccc3c(ccc4ccccc43)c2)cc1 ; c1ccc(-c2ccccc2N(c2cccc(-c3cc4ccccc4c4c3ccc3ccccc34)c2)c2cc3ccccc3c3ccccc23)cc1]

9. An organic light emitting device comprising:
a first electrode;
a second electrode that is provided opposite to the first electrode; and
one or more organic material layers that are provided between the first electrode and the second electrode, wherein one or more layers of the organic material layers comprises the compound as set forth in claim 1.

10. The organic light emitting device of claim 9, wherein the organic material layer is an electron blocking layer.
